(19) United States
(12) Patent Application Publication
(10) Pub. No.: US 2025/0135025 A1
(43) Pub. Date: May 1, 2025

(54) ANTI-ROR1 ANTIBODY CONJUGATES, COMPOSITIONS COMPRISING ANTI ROR1 ANTIBODY CONJUGATES, AND METHODS OF MAKING AND USING ANTI-ROR1 ANTIBODY CONJUGATES

(71) Applicant: Sturo Biopharma, Inc., South San Francisco, CA (US)

(72) Inventors: Amandeep Gakhal, San Mateo, CA (US); Abigail Yu, San Jose, CA (US); Ryan Stafford, Foster City, CA (US); Jeffrey Hanson, Oakland, CA (US); Alice Yam, Belmont, CA (US); Krishna Bajjuri, Union City, CA (US); Andreas Maderna, Walnut Creek, CA (US); Christina Abrahams, Burlingame, CA (US); Xiaofan Li, Belmont, CA (US); Gang Yin, South San Francisco, CA (US); Miao Wen, Union City, CA (US); Kristin Bedard, Bellevue, WA (US); Daniel Calarese, Millbrae, CA (US); Helena Kiefel, South San Francisco, CA (US)

(21) Appl. No.: 19/005,430

(22) Filed: Dec. 30, 2024

Related U.S. Application Data
(63) Continuation of application No. 18/971,438, filed on Dec. 6, 2024.
Application No. 18/971,438 is a division of application No. 18/349,337, filed on Jul. 10, 2023, now Pat. No. 12,226,490.
Application No. 18/349,337 is a continuation of application No. PCT/US2023/026769, filed on Jun. 30, 2023.
(60) Provisional application No. 63/357,442, filed on Jun. 30, 2022.
(60) Provisional application No. 63/389,741, filed on Jul. 15, 2022.
(60) Provisional application No. 63/382,262, filed on Nov. 3, 2022.
(60) Provisional application No. 63/487,706, filed on Mar. 1, 2023.
(60) Provisional application No. 63/489,926, filed on Mar. 13, 2023.
(60) Provisional application No. 63/495,635, filed on Apr. 12, 2023.

(51) Int. Cl. A61K 47/68; A61K 31/502; A61P 35/00

(52) U.S. Cl. CPC A61K 47/6869; A61K 31/502; A61K 47/6803; A61K 47/6805; A61K 47/6809; A61K 47/6849; A61K 47/6889; A61P 35/00

(57) ABSTRACT

The present disclosure relates to antibodies and antibody conjugates, for instance antibody drug conjugates, with binding specificity for receptor tyrosine kinase orphan receptor 1 (ROR1) and its isoforms and homologs, and compositions comprising the antibodies or antibody conjugates, including pharmaceutical compositions. Also provided are methods of producing the antibodies and antibody conjugates and compositions thereof as well as methods of using the antibodies and antibody conjugates and compositions thereof, such as in therapeutic and diagnostic methods.

### CROSS-REFERENCE TO RELATED APPLICATIONS

This application is a continuation U.S. patent application Ser. No. 18/971,438 filed Dec. 6, 2024, which is a divisional of U.S. patent application Ser. No. 18/349,337 filed Jul. 10, 2023, which is a continuation of PCT/US23/26769 file Jun. 30, 2023, which claims the benefit of U.S. Provisional Application No. 63/357,442 filed Jun. 30, 2022; U.S. Provisional Application No. 63/389,741 filed Jul. 15, 2022; U.S. Provisional Application No. 63/382,262 filed Nov. 3, 2022; U.S. Provisional Application No. 63/487,706 filed Mar. 1, 2023; U.S. Provisional Application No. 63/489,926 filed Mar. 13, 2023; and U.S. Provisional Application No. 63/495,635 filed Apr. 12, 2023. Each of these applications are incorporated for all purposes in their entirety.

This application incorporates by reference the computer readable sequence listing entitled “108843.00540 Sequence Listing” created Jul. 30, 2023 having 1.29 MB.

### FIELD OF THE INVENTION

Provided herein are antibodies and antibody conjugates, for instance antibody drug conjugates, with binding specificity for receptor tyrosine kinase orphan receptor 1 (ROR1) and compositions comprising the antibodies or antibody conjugates, including pharmaceutical compositions. Also provided herein are methods of producing the antibodies and conjugates, and methods of using the antibodies or conjugates and compositions for therapy. The antibodies, conjugates, and compositions are useful in methods of treatment and prevention of cell proliferation and cancer, for example, by activating anti-tumor immunity; methods of detection of cell proliferation and cancer; and methods of diagnosis of cell proliferation and cancer. The antibodies, conjugates, and compositions are also useful in methods of treatment, prevention, detection, and diagnosis of autoimmune diseases, infectious diseases, and inflammatory conditions.

### BACKGROUND

Receptor tyrosine kinase-like orphan receptor (ROR1) is a member of the receptor tyrosine kinases (RTK) family. OMIM entry 602336. Receptor tyrosine kinases function as cell surface receptors, and they have been postulated to play roles in the control of cell proliferation, differentiation, migration, and metabolism. Afzal & Jeffery, 2003, Hum. Mutat. 22:1-11. Based on conserved sequences from other receptor tyrosine kinases, ROR1 was initially identified in 1992. Masiakowski & Carroll, 1992, J. Biol. Chem. 267:26181-26190. The ROR1 gene encodes a type I glycosylated membrane protein with a predicted length of 937 amino acids. See id.; Entrez Gene ID. 4919. The ROR1 gene has been mapped to chromosome 1p32-p31. Reddy et al., 1997, Genomics 41:283-285. Expression of ROR1 has been observed in human heart, lung, and kidney, and also weakly in the central nervous system. Reddy et al. Truncated expression was observed in a variety of human cancers, including those originating from CNS or PNS neuroectoderm. Reddy, 1996, Oncogene 13:1555-1559. ROR1 knockout mice had no obvious skeletal or cardiac abnormalities, yet they died soon after birth due to respiratory dysfunction. Nomi et al., 2001, Molec. Cell. Biol. 21:8329-8335.

ROR1 has recently been shown to be expressed on cancer cells, including ovarian cancer cells, and on cancer stem cells. Zhang et al., 2014, Proc. Natl. Acad. Sci. USA 111:17266 71. Treatment with a monoclonal antibody specific for ROR1 inhibited development of ovarian cancer cells, and ROR1 has been postulated as a target for cancer therapeutics. See id.

There is a need for improved methods of targeting and/or modulating the activity of ROR1. Given the specific expression of ROR1 in cancer cells and cancer stem cells, there is a need for improved therapeutics that can specifically target cells and tissues that express or overexpress ROR1. Antibody conjugates to ROR1 could be used to deliver therapeutic or diagnostic payload moieties to target cells expressing ROR1 for the treatment or diagnosis of such diseases.

### SUMMARY

Provided herein are antibodies that selectively bind ROR1. In some embodiments, the antibodies bind human ROR1. In some embodiments, the antibodies also bind homologs of human ROR1.

In some embodiments, the antibodies comprise an illustrative CDR, V_{H}, or V_{L} sequence provided in this disclosure, or a variant thereof. In some aspects, the variant is a variant with one or more conservative amino acid substitutions. In some aspects, the variant has sequence identity to the illustrative sequence or sequences.

Also provided are compositions and kits comprising the antibodies. In some embodiments, the compositions are pharmaceutical compositions. Any suitable pharmaceutical composition may be used. In some embodiments, the pharmaceutical composition is a composition for parenteral administration.

This disclosure also provides methods of using the anti-ROR1 antibodies provided herein. In some embodiments, the method is a method of treatment. In some embodiments, the method is a diagnostic method. In some embodiments, the method is an analytical method. In some embodiments, the method is a method of purifying and/or quantifying ROR1.

In some embodiments, the antibodies are used to treat a disease or condition. In some aspects, the disease or condition is selected from cancer, autoimmune disease, and infection.

Also provided herein are antibody conjugates that selectively bind receptor tyrosine kinase orphan receptor 1 (ROR1). The antibody conjugates comprise an antibody that binds ROR1 linked to one or more payload moieties. The antibody can be linked to the payload directly by a covalent bond or indirectly by way of a linker. ROR1 antibodies are described in detail herein, as are useful payload moieties, and useful linkers.

In another aspect, provided are compositions comprising the antibody conjugates. In some embodiments, the compositions are pharmaceutical compositions. Any suitable pharmaceutical composition may be used. In some embodiments, the pharmaceutical composition is a composition for parenteral administration. In a further aspect, provided herein are kits comprising the antibody conjugates or pharmaceutical compositions.

In another aspect, provide herein are methods of using the anti-ROR1 antibody conjugates. In some embodiments, the methods are methods of delivering one or more payload moieties to a target cell or tissue expressing ROR1. In some embodiments, the methods are methods of treatment. In some embodiments, the methods are diagnostic methods. In some embodiments, the methods are analytical methods. In some embodiments, the antibody conjugates are used to treat a disease or condition. In some aspects, the disease or condition is selected from a cancer, autoimmune disease, and infection. In certain embodiments, the anti-ROR1 antibody conjugates treat the disease or condition, for example cancer, by activating anti-tumor immunity or protective immunity.

In some embodiments, the antibody conjugates bind human ROR1. In some embodiments, the antibody conjugates also bind homologs of human ROR1. In some aspects, the antibody conjugates also bind homologs of cynomolgus monkey and/or mouse receptor ROR1.

These and other embodiments of the invention along with many of its features are described in more detail in conjunction with the text below and attached figures.

### BRIEF DESCRIPTION OF THE FIGURES

FIG. 1 provides a comparison of the Kabat and Chothia numbering systems for CDR-H1. Adapted from Martin A. C. R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.

FIGS. 2-6 provide alignments of the V_{H} sequences (SEQ ID NOs: 854-1020) from the variant antibodies provided herein. CDRs according to Chothia are highlighted, and CDRs according to Kabat are boxed.

FIG. 7 provides alignments of the V_{L} sequences (SEQ ID NOs: 1021-1026) from trastuzumab and the variant antibodies provided herein. CDRs according to Chothia are highlighted, and CDRs according to Kabat are underlined.

FIGS. 8A-8C provide ELISA binding curves for select heavy chains with three different light chains.

FIGS. 9A-9H provide the results of kinetic screens for select heavy chains with four different light chains.

FIG. 10A provides formulation buffer comparison for Conjugate 46 at 4° C. conditions. FIG. 10B provides formulation buffer comparison for Conjugate 46 at 25° C. and 37° C. conditions. FIG. 10C provides formulation buffer comparison for Conjugate 47 at 4° C. conditions. FIG. 10D provides formulation buffer comparison for Conjugate 47 at 25° C. and 37° C. conditions. FIG. 10E provides HPLC-SEC results over 5× freeze/thaw cycles for Conjugate 46. FIG. 10F provides HPLC-SEC results over 5× freeze/thaw cycles for Conjugate 47. FIG. 10G provides protein concentration results over 5× freeze/thaw cycles for Conjugate 46. FIG. 10H provides protein concentration results over 5× freeze/thaw cycles for Conjugate 47. FIG. 10I and FIG. 10J provide SEC monomer % with increasing protein concentrations, 3-week hold at 4° C.

FIG. 11A provides calculated DAR of Conjugate 47 over 7 days. FIG. 11B provides calculated DAR of Conjugate 46 over 7 days. FIG. 11C provides calculated DAR of Conjugate 48 over 7 days. FIG. 11D provides calculated DAR of Conjugate 50 over 7 days. FIG. 11E provides deconvoluted mass spectra of aROR1 ADC samples from in vivo linker payload stability study for Conjugate 46. FIG. 11F provides deconvoluted mass spectra of aROR1 ADC samples from in vivo linker payload stability study for Conjugates 47. FIG. 11G provides deconvoluted mass spectra of aROR1 ADC samples from in vivo linker payload stability study for Conjugate 48. FIG. 11H provides deconvoluted mass spectra of aROR1 ADC samples from in vivo linker payload stability study for Conjugates 50.

FIG. 12A and FIG. 12B demonstrate that Conjugate 46 and Conjugate 47 showed potent cell killing on ROR1 positive Ntera-2 cells, while anti-GFP antibody conjugated to LP3 or LP4 showed no detected cell killing on Ntera-2 cells at all. FIG. 12C and FIG. 12D demonstrate that in the presence of 1 μM of the un-conjugated anti-ROR1 antibody 2188-D04, the cell killing activity of both Conjugate 46 and Conjugate 47 was inhibited.

FIG. 13A-C provide MDA-MB-231 tumor growth curves in response to treatment with four weekly doses (qwx4) of ROR1-targeted ADCs, with doses ranging from (11A) 2 mg/kg to (11B) 5 mg/kg. (11C) Scatter plot of individual tumor volumes on day 42 post treatment, when control tumors reached the study endpoint. Arrows represent dosing days. Statistical analysis was performed on tumor volumes on day 42 using one-way ANOVA with Dunnett's multiple comparisons test versus the vehicle group. A probability of less than 5% (p<0.05) was considered significant. ***=p<0.001; ****=p<0.0001. All graphs are presented as individual values or mean±SEM.

FIGS. 14A-C provide MDA-MB-231 tumor growth curves in response to treatment with four weekly doses (qwx4) of ROR1-targeted ADCs, with doses ranging from (12A) 2 mg/kg to (12B) 5 mg/kg. (12C) Scatter plot of individual tumor volumes on day 43 post treatment, when control tumors reached the study endpoint. Arrows represent dosing days. Statistical analysis was performed on tumor volumes on day 43 using one-way ANOVA with Dunnett's multiple comparisons test versus the vehicle group. A probability of less than 5% (p<0.05) was considered significant. ****=p<0.0001. All graphs are presented as individual values or mean±SEM.

FIGS. 15A-C provide MDA-MB-231 tumor growth curves in response to treatment with three weekly doses (qwx3) of ROR1-targeted ADCs, with doses ranging from (13A) 2 mg/kg to (13B) 5 mg/kg. (13C) Scatter plot of individual tumor volumes on day 37 post treatment, when control tumors reached the study endpoint. Arrows represent dosing days. Statistical analysis was performed on tumor volumes on day 37 using one-way ANOVA with Dunnett's multiple comparisons test versus the vehicle group. A probability of less than 5% (p<0.05) was considered significant. *=p<0.05; **=p<0.01; ***=p<0.001; ****=p<0.0001. All graphs are presented as individual values or mean±SEM.

FIGS. 16A-C provide MDA-MB-231 tumor growth curves in response to treatment with four weekly doses (qwx4) of ROR1-targeted ADCs (14A) Conjugate 49 and (14B) Conjugate 46, with doses ranging from 1 mg/kg to 5 mg/kg. (14C) Scatter plot of individual tumor volumes on study day 29, when control tumors reached the study endpoint. Arrows represent dosing days. Statistical analysis was performed on tumor volumes on study day 29 using one-way ANOVA with Dunnett's multiple comparisons test versus the vehicle group. A probability of less than 5% (p<0.05) was considered significant. *=p<0.05; **=p<0.01; ***=p<0.001; ****=p<0.0001. All graphs are presented as individual values or mean±SEM.

FIGS. 17A-D provide H1975 tumor growth curves in response to treatment with two weekly doses (qwx2) of ROR1-targeted ADCs (15A) Conjugate 46, (15B) Conjugate 47, and (15C) Conjugate 44, with doses ranging from 1 mg/kg to 10 mg/kg. (15D) Scatter plot of individual tumor volumes on study day 14, when control tumors reached the study endpoint. Arrows represent dosing days. Statistical analysis was performed on tumor volumes on study day 14 using one-way ANOVA with Dunnett's multiple comparisons test versus the vehicle group. A probability of less than 5% (p<0.05) was considered significant. **=p<0.01. All graphs are presented as individual values or mean±SEM.

FIGS. 18A-I provide PDX tumor growth curves in response to treatment with up to five weekly doses (qwx5) of ROR1-targeted ADCs Conjugate 46, Conjugate 47, and Conjugate 49 dosed at 10 mg/kg in different NSCLC PDx models. Arrows represent dosing days. All graphs are presented as mean±SEM.

FIGS. 19A-D provide PDX tumor growth curves in response to treatment with up to three weekly doses (qwx3) of Conjugate 46 dosed at 5 mg/kg and twenty-eight daily doses (qdx28) of olaparib dosed at 50 mg/kg in different TNBC PDX models. Arrows represent dosing days for Conjugate 46; dotted line represents dosing days for olaparib. All graphs are presented as mean±SEM.

FIG. 20A provides calreticulin expression in Ntera-2 cells following treatment with Conjugate 46 or exatecan compared to the antibody alone and an isotype control of Conjugate 46.

FIG. 20B provides HMGB1 release in Ntera-2 cells following treatment with Conjugate 46 or exatecan compared to the antibody alone and an isotype control of Conjugate 46.

FIG. 21A provides percent of live target cells in Ntera-2 cells following treatment with Conjugate 46 or exatecan compared to the antibody alone and an isotype control of Conjugate 46.

FIG. 21B is a graph showing monocyte activation in Ntera-2 cells following treatment with Conjugate 46 or exatecan compared to the antibody alone and an isotype control of Conjugate 46.

FIG. 21C is a graph showing the percent of calreticulin on the cell surface of Ntera-2 cells following treatment with Conjugate 46 or exatecan compared to the antibody alone and an isotype control of Conjugate 46. Calreticulin surface expression is measured as a percentage compared to untreated cells.

FIGS. 22A-22F provide bar graphs of proportions of (FIG. 22A) total T cells, (FIG. 22B) CD4+ T cells, (FIG. 22C) CD8+ T cells, (FIG. 22D) and tumor-associated macrophages (TAMs), (FIG. 22E) CD80 median fluorescence intensity (MFI) on TAMs, and proportion of (FIG. 22F) arginase 1+ (Arg1+) TAMs in the tumors of mice treated with vehicle or 10 mg/kg Conjugate 46 (qwx2) and/or 8 mg/kg anti-PD-1 (q3dx3). All graphs are presented as individual values and mean±SEM.

### DETAILED DESCRIPTION OF THE EMBODIMENTS

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The term “combinations thereof” includes every possible combination of elements to which the term refers to.

The terms “ROR1” and “receptor tyrosine kinase like orphan receptor 1” are used interchangeably herein. ROR1 is also known by synonyms, including NTRKR1 neurotrophic tyrosine kinase, receptor-related 1, and dJ537F10.1, among others. Unless specified otherwise, the terms include any variants, isoforms and species homologs of human ROR1 that are naturally expressed by cells, or that are expressed by cells transfected with a ROR1 or ROR1 gene. ROR1 proteins include, for example, human ROR1 (SEQ ID NO: 1). In some embodiments, ROR1 proteins include cynomolgus monkey ROR1 (SEQ ID NO: 2). In some embodiments, ROR1 proteins include murine ROR1 (SEQ ID NO: 3).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (CH). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated CL.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer. A “ROR1 antibody,” “anti-ROR1 antibody,” “ROR1 Ab,” “ROR1-specific antibody,” “anti-ROR1 Ab,” “ROR1 antibody,” “anti-ROR1 antibody,” “ROR1 Ab,” “ROR1-specific antibody,” or “anti ROR1 Ab” is an antibody, as described herein, which binds specifically to ROR1 or ROR1. In some embodiments, the antibody binds the extracellular domain of ROR1.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 | H31-H35B | H26-H32 |
| (Kabat Numbering) | | or H34* |
| H1 | H31-H35 | H26-H32 |
| (Chothia Numbering) | | |
| H2 | H50-H65 | H52-H56 |
| H3 | H95-H102 | H95-H102 |

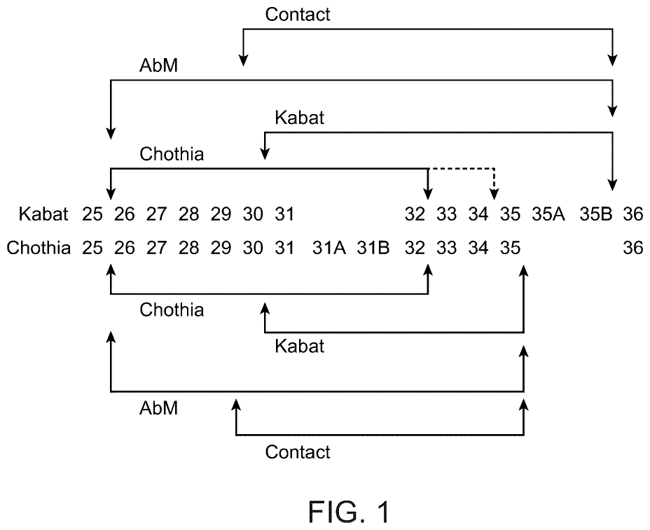

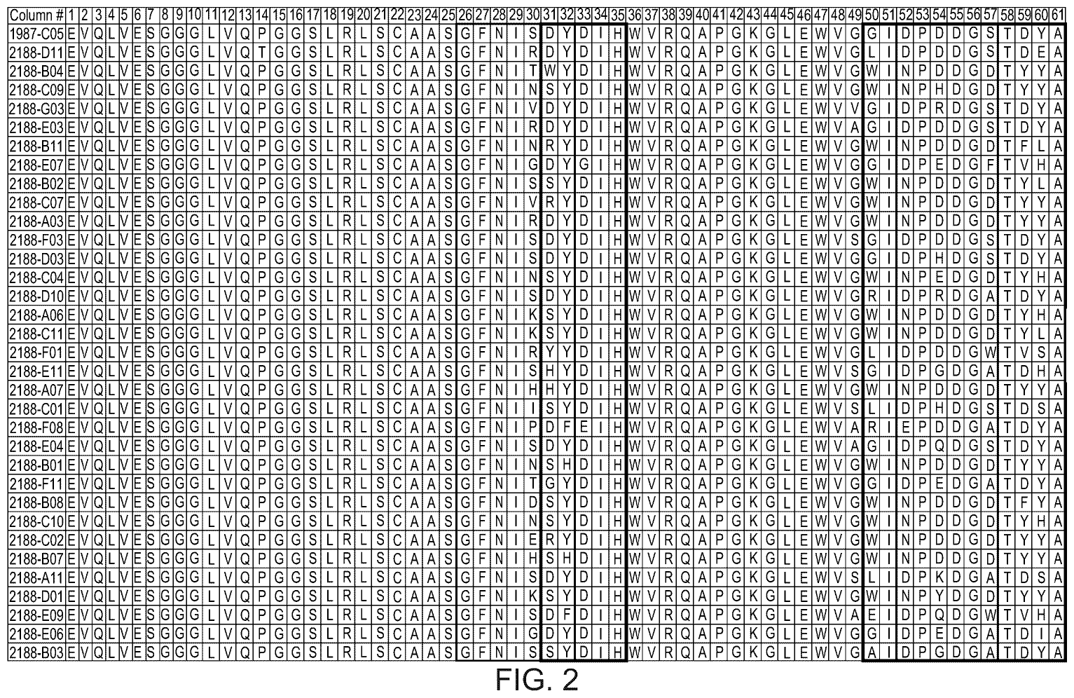

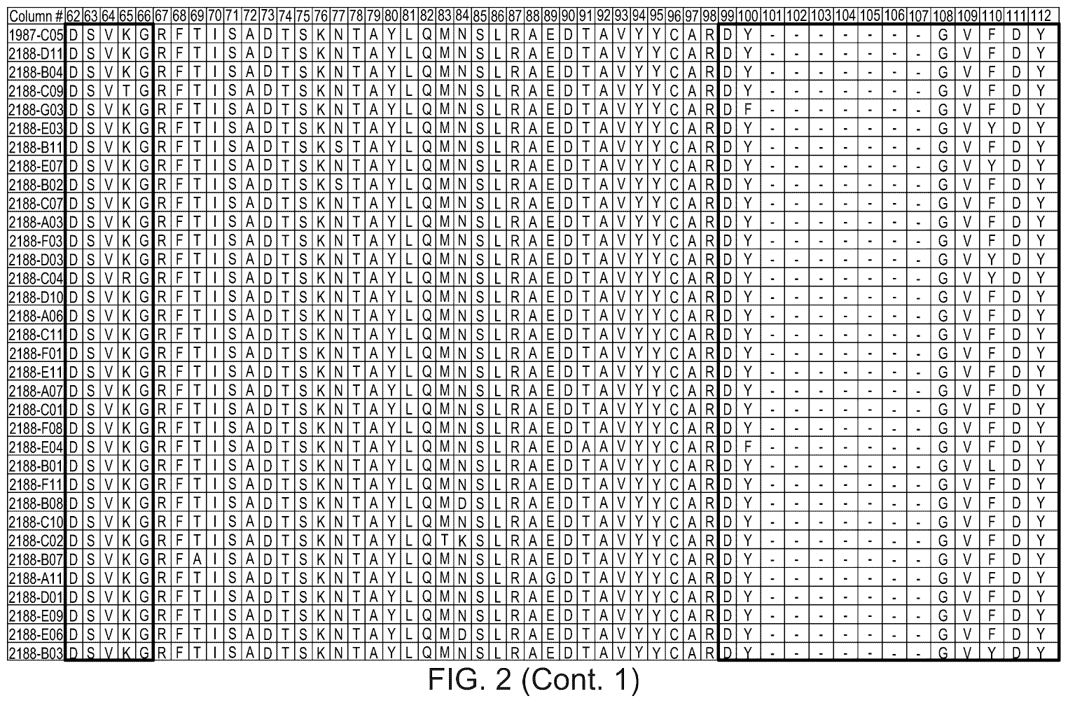

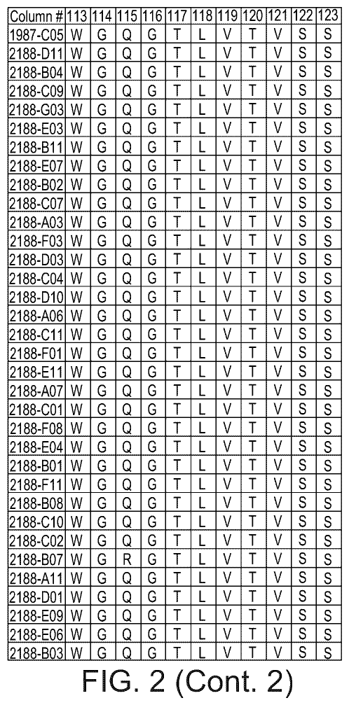

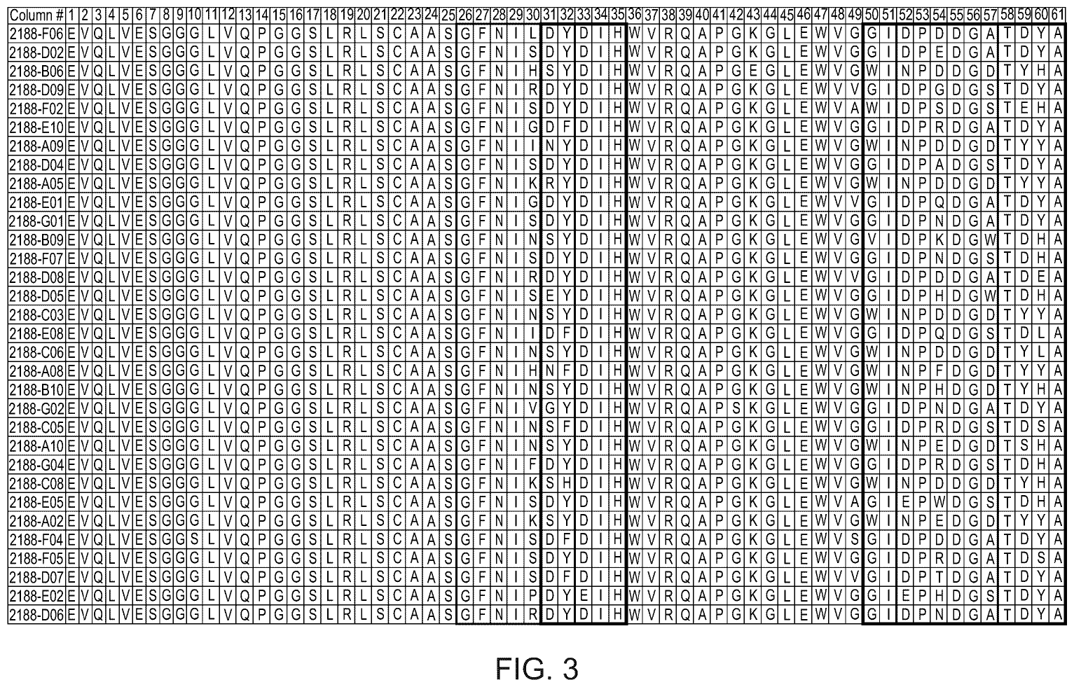

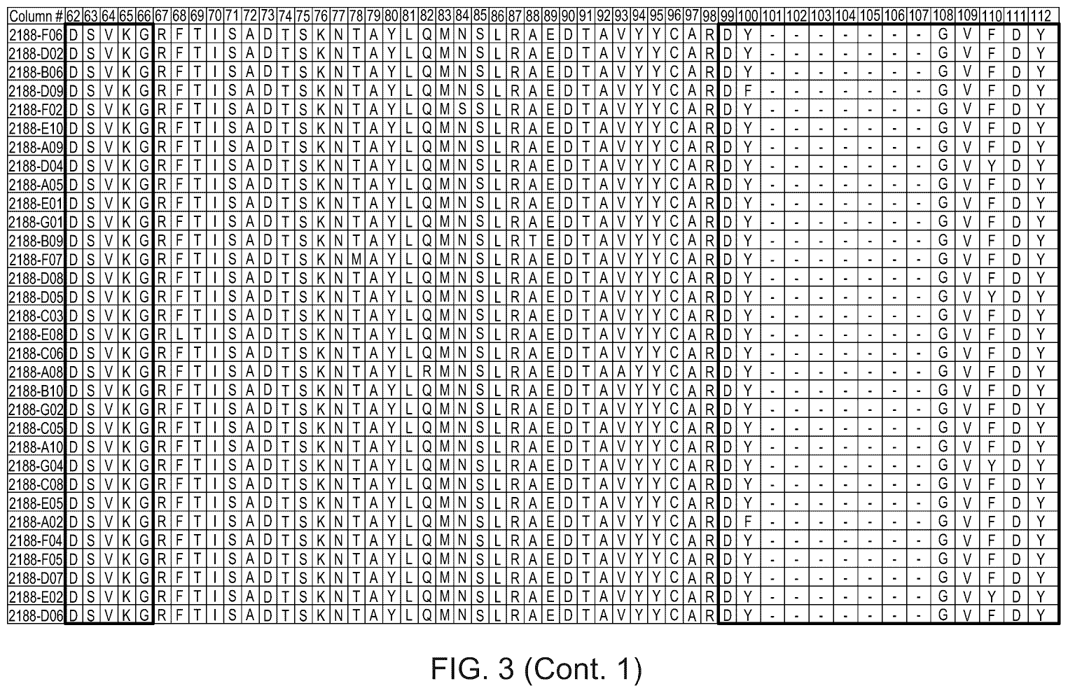

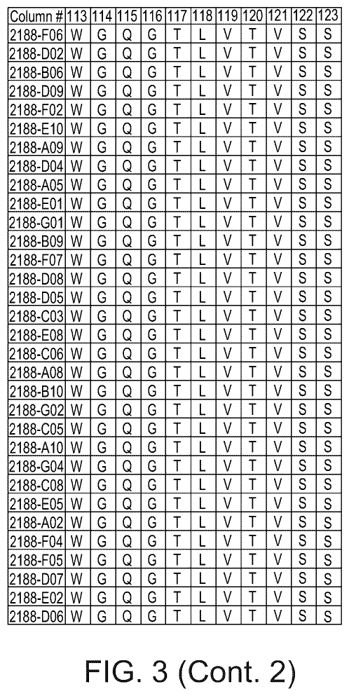

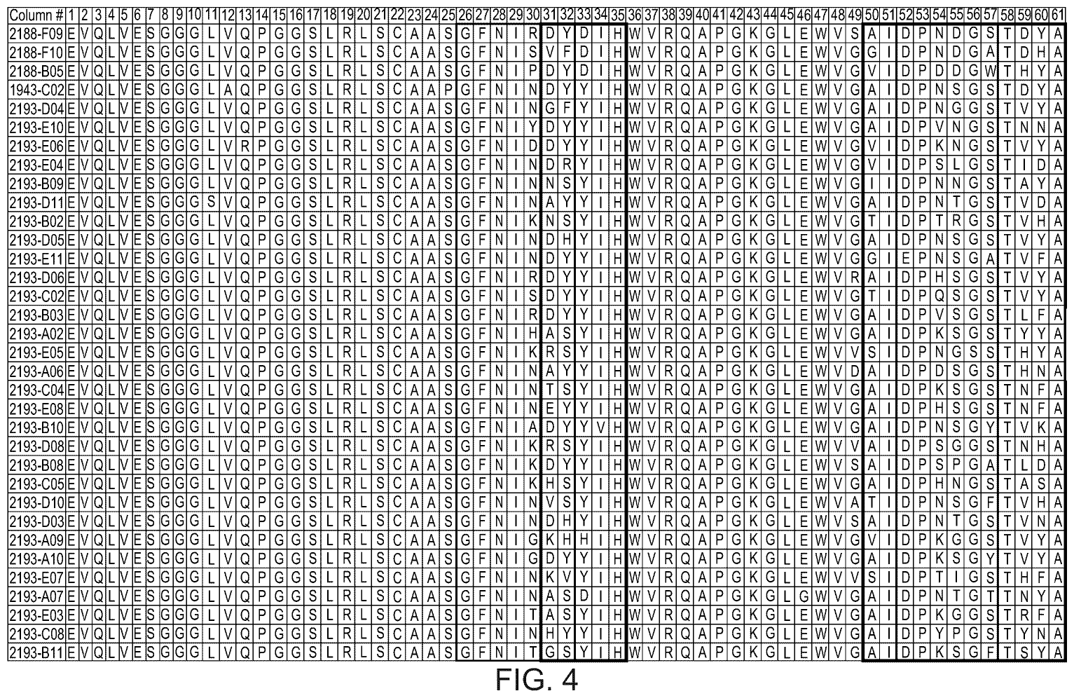

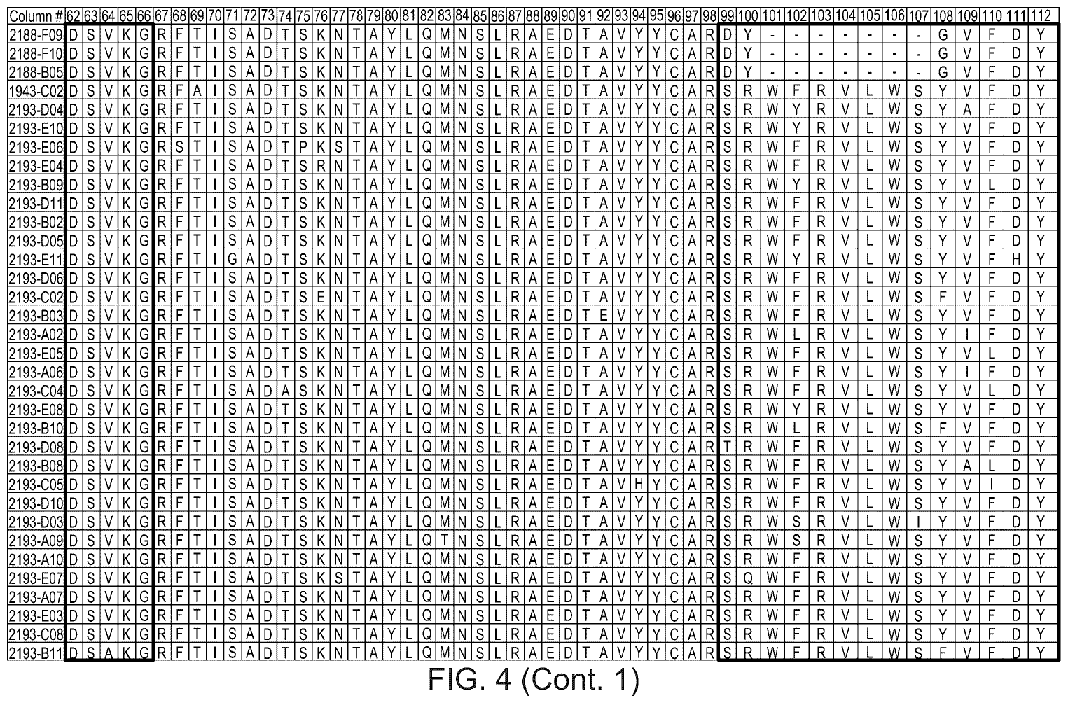

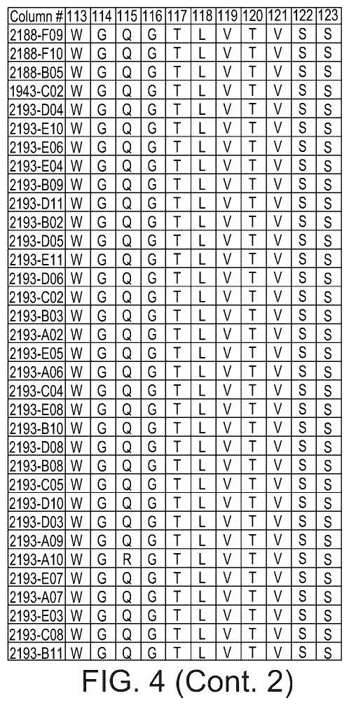

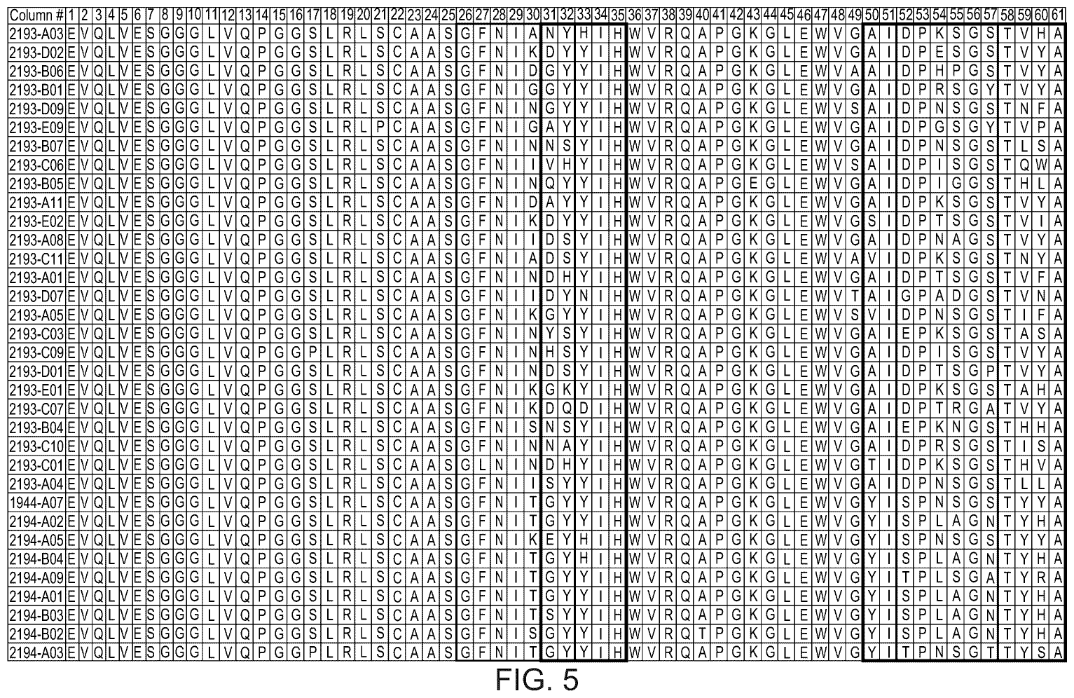

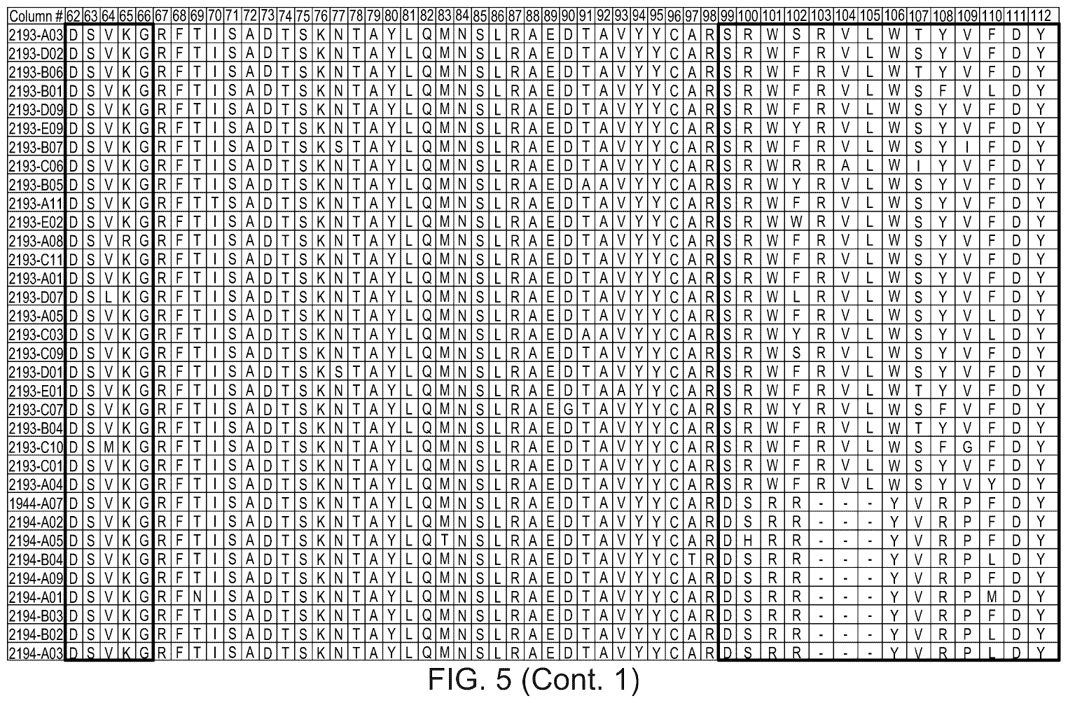

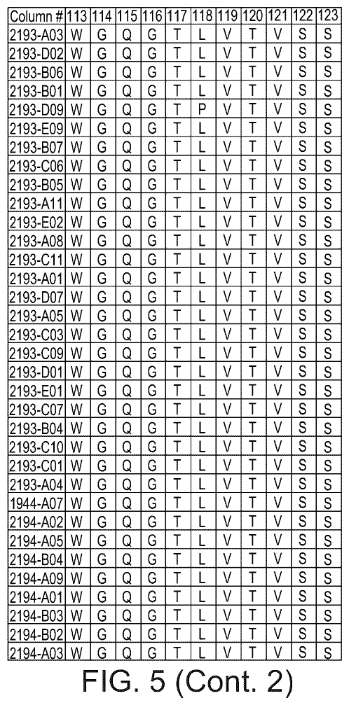

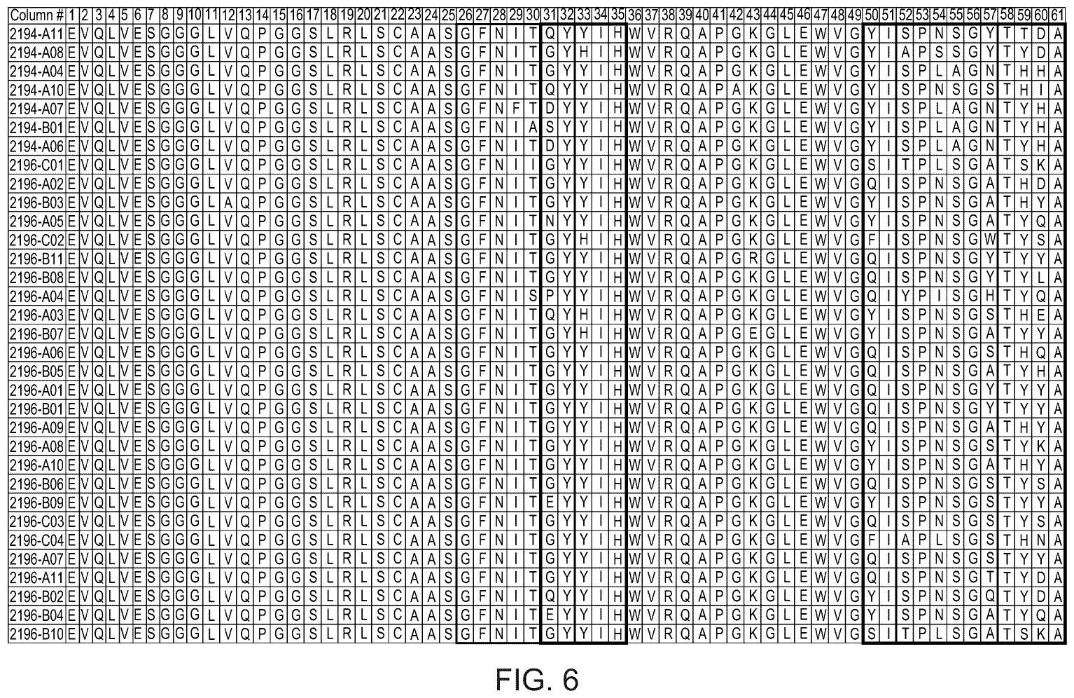

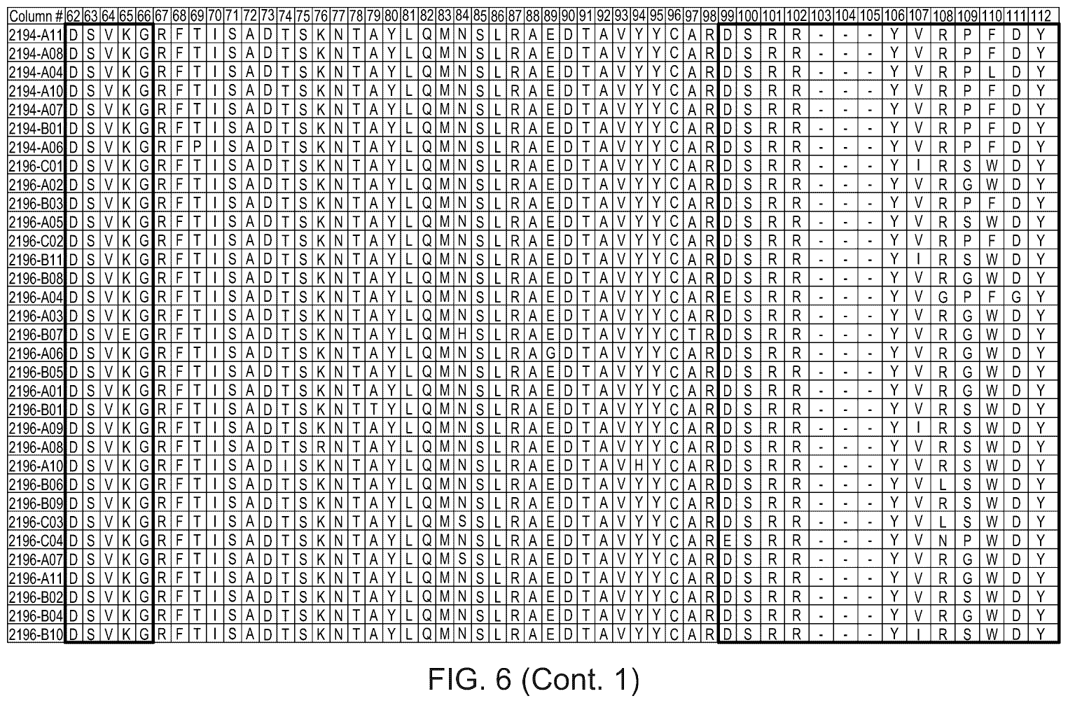

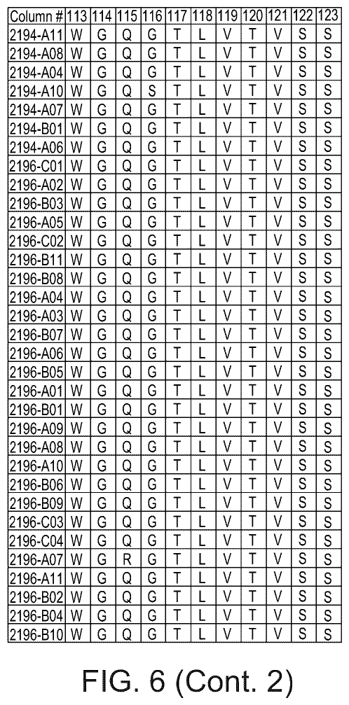

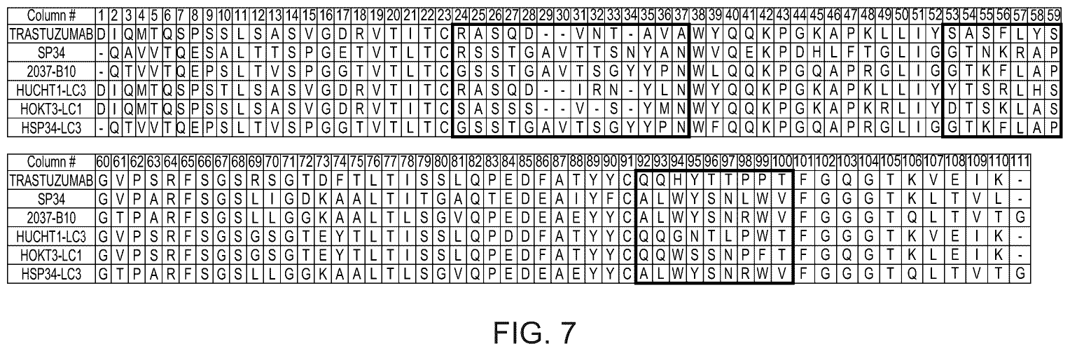

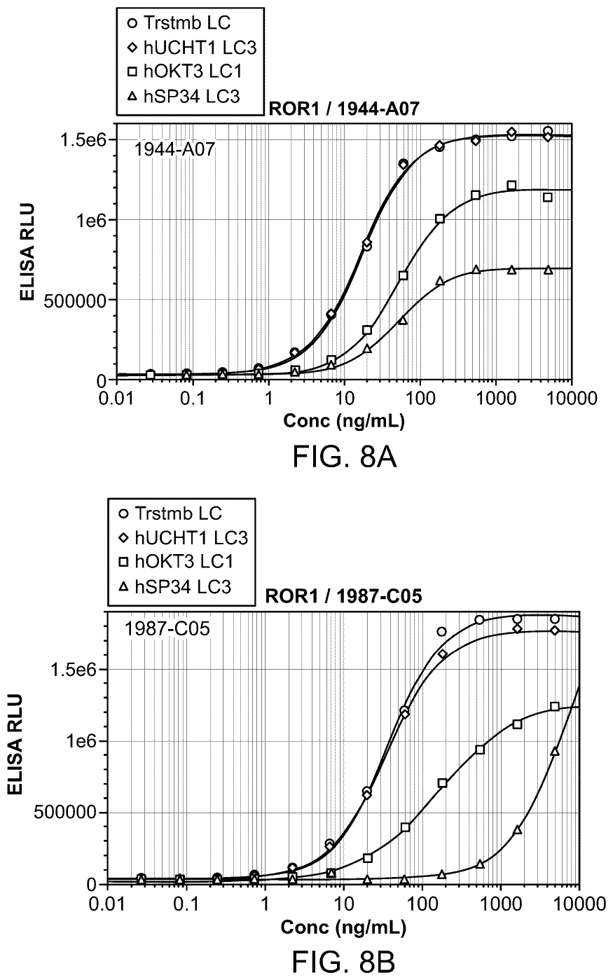

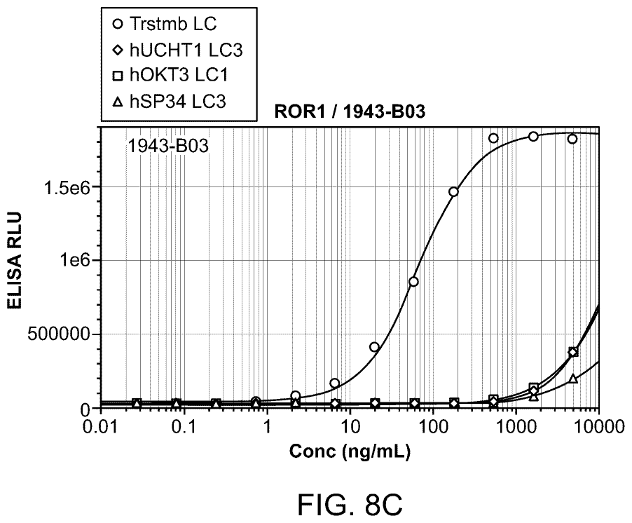

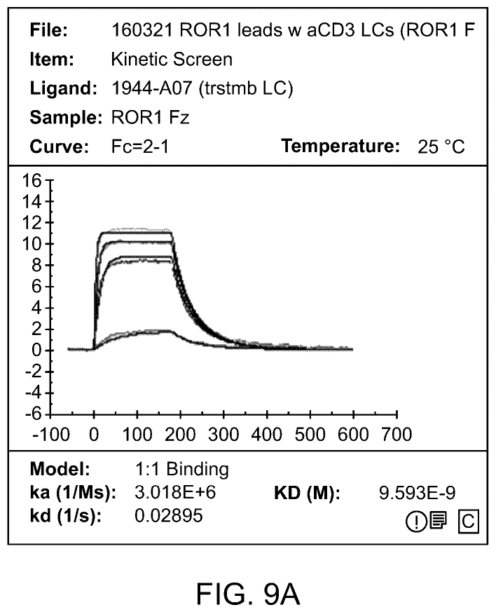

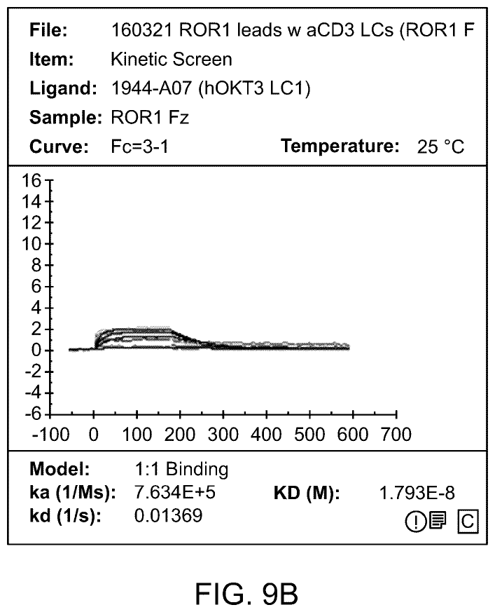

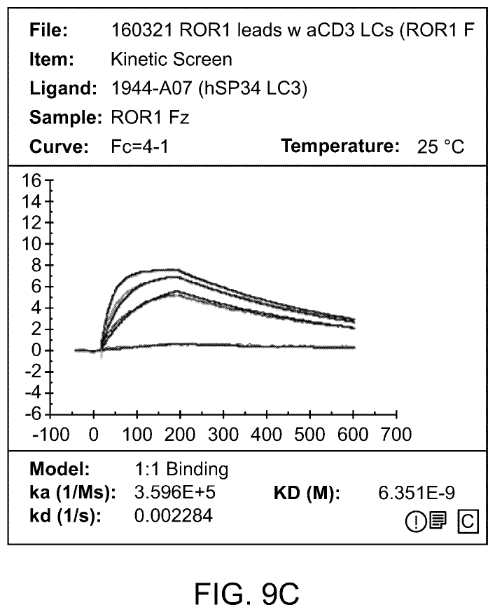

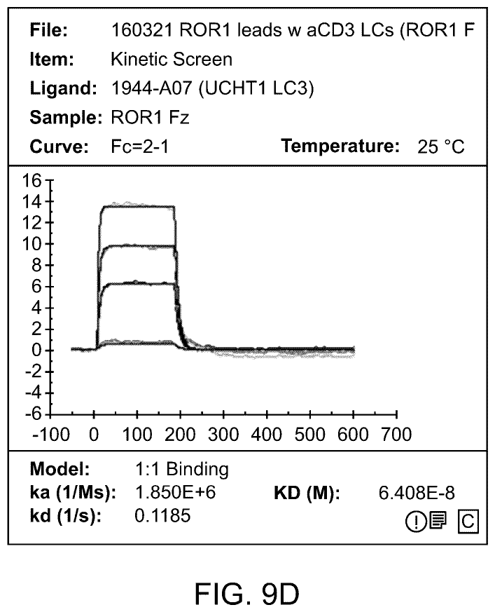

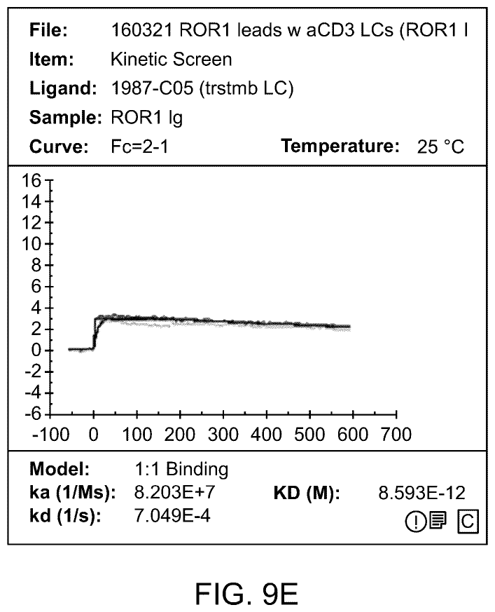

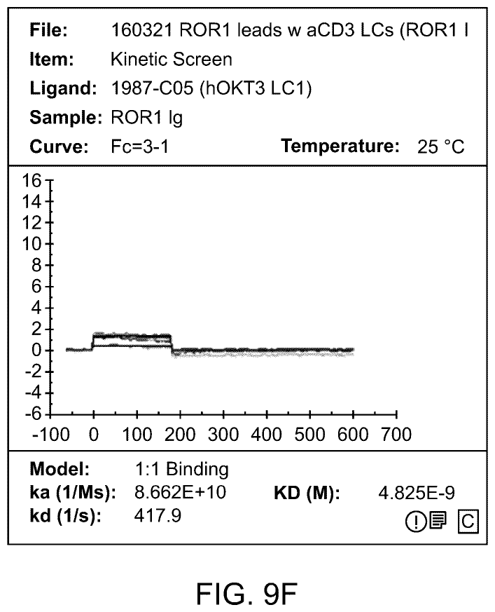

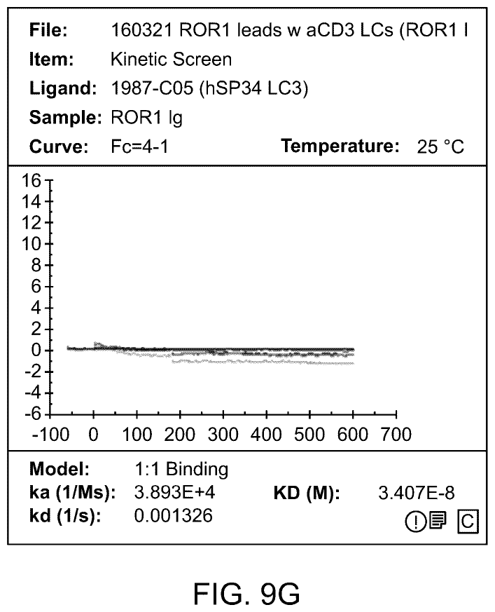

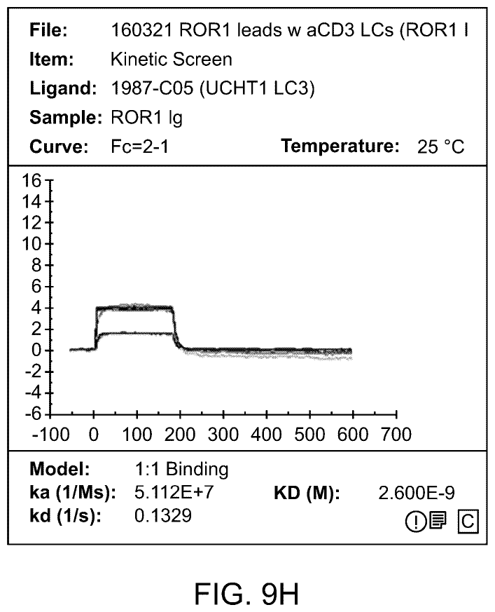

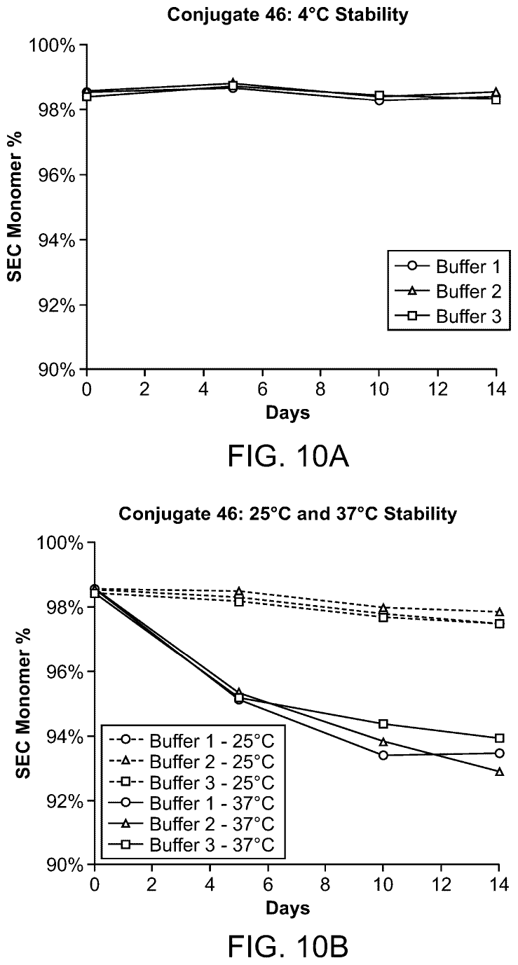

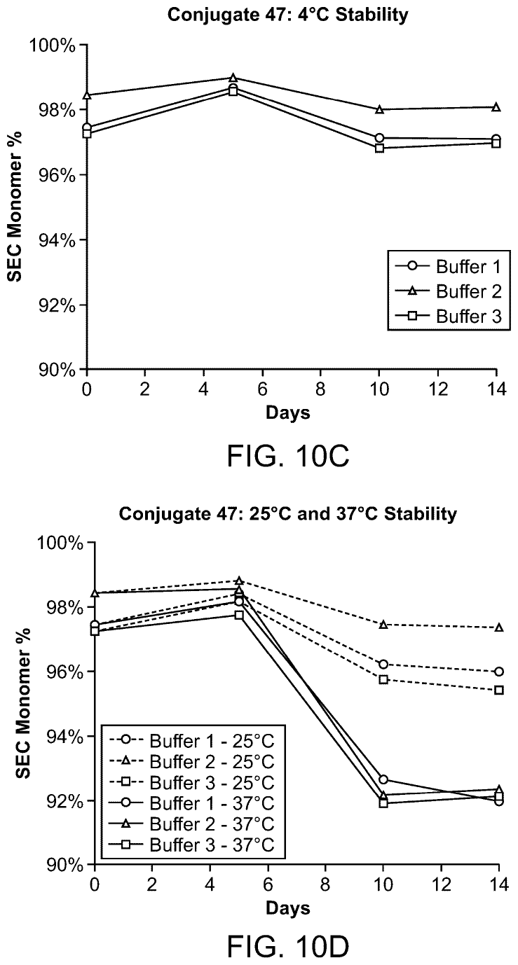

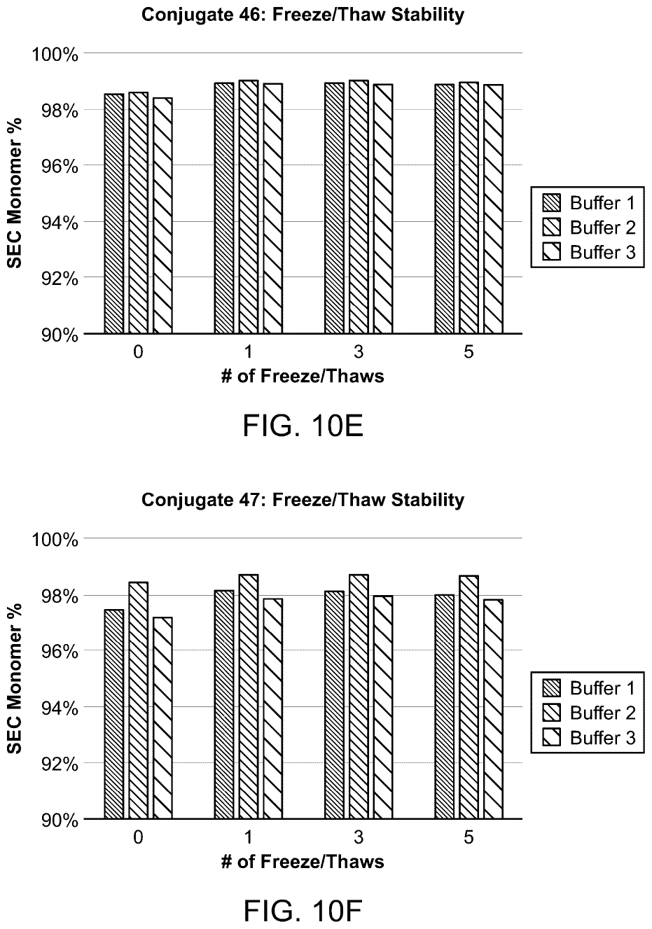

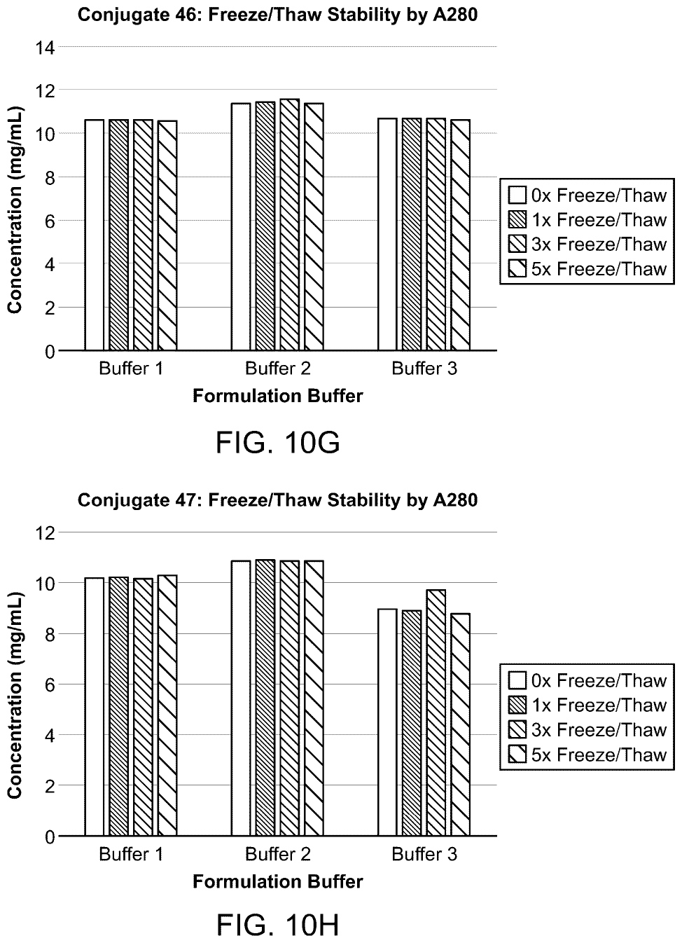

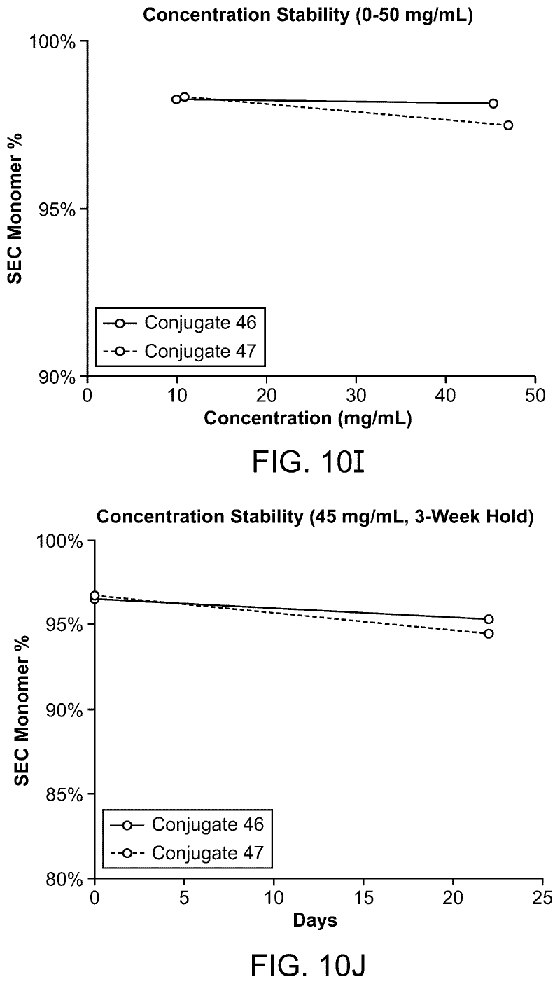

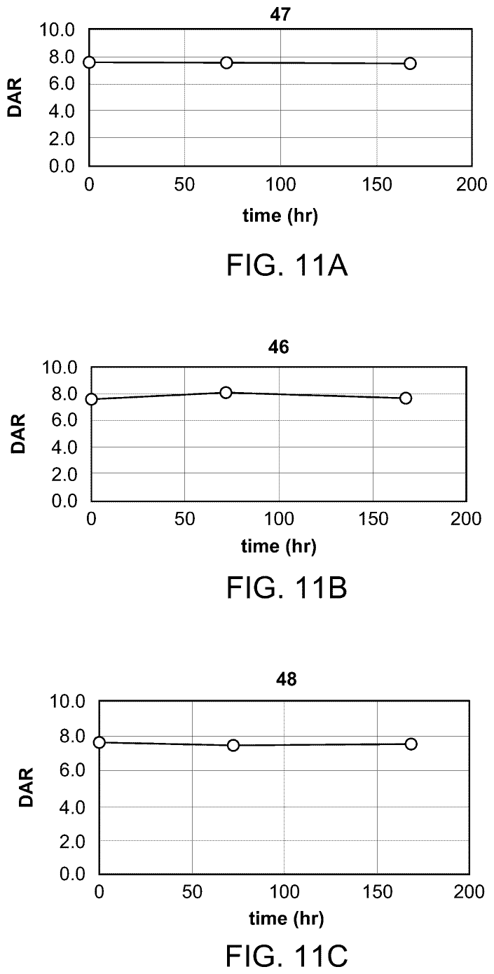

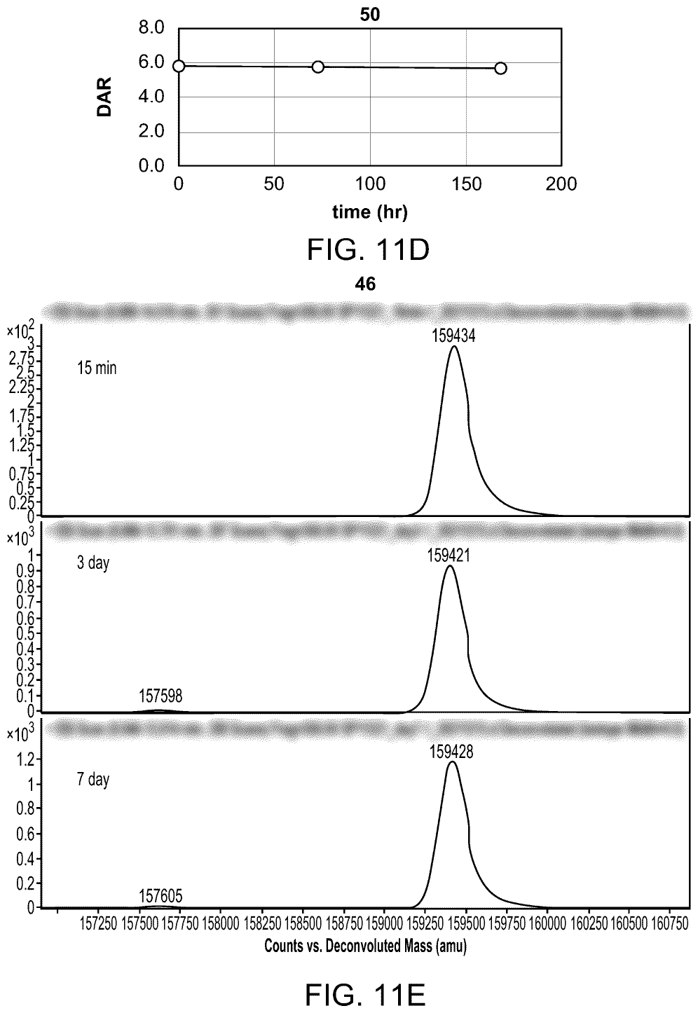

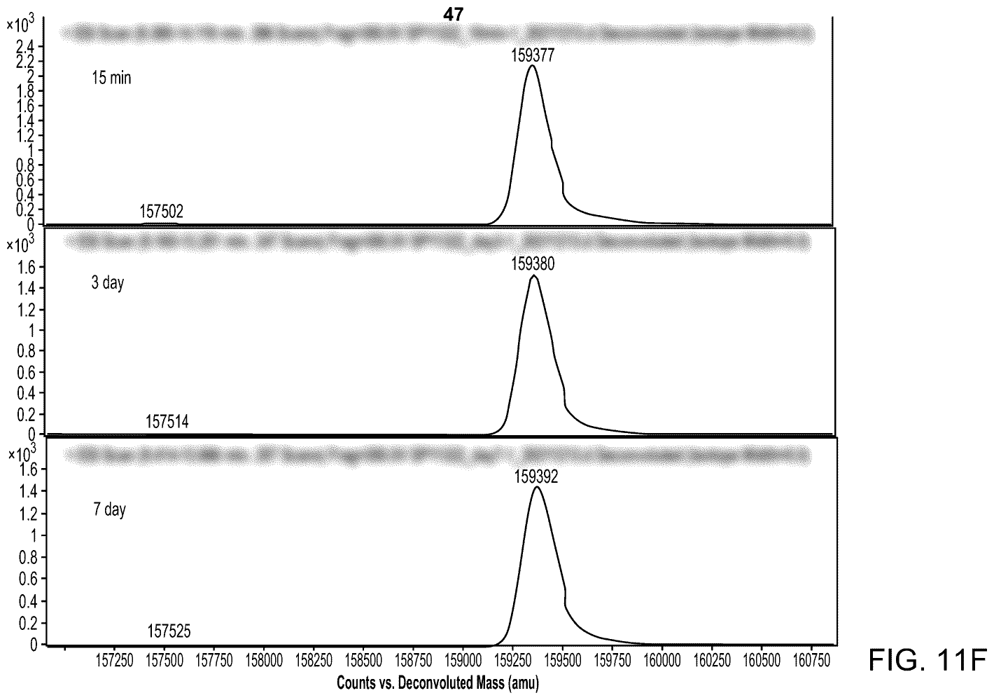

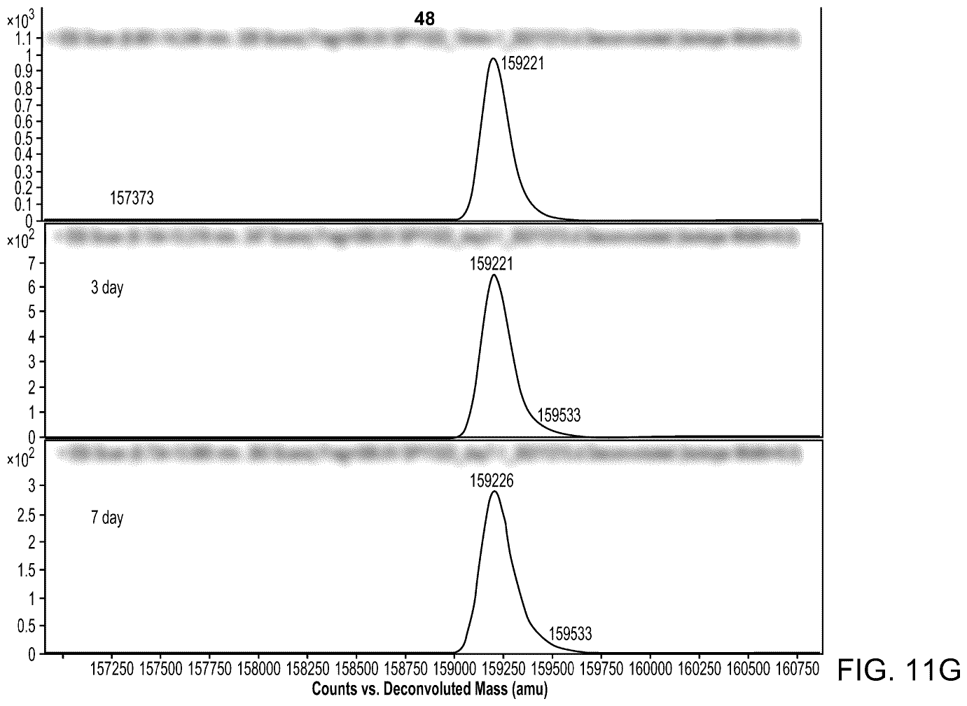

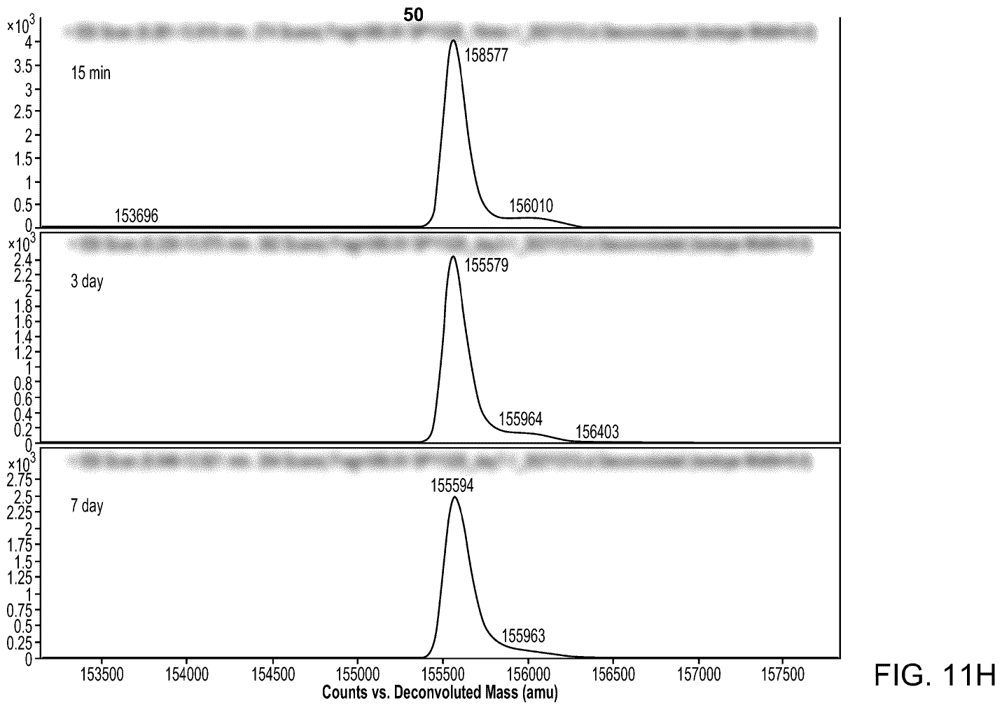

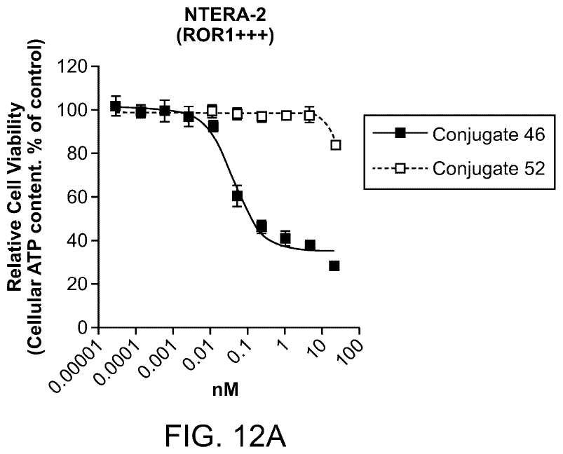

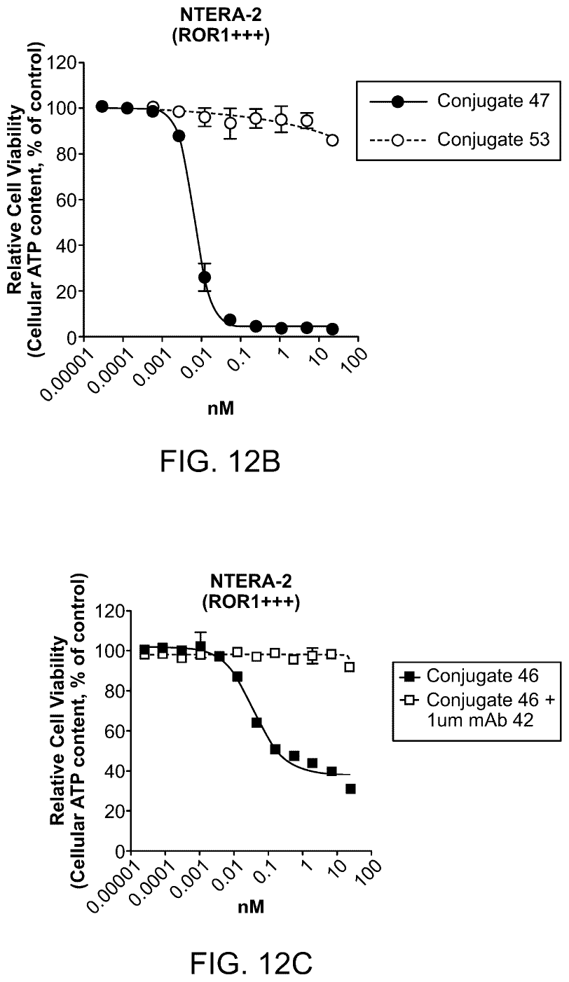

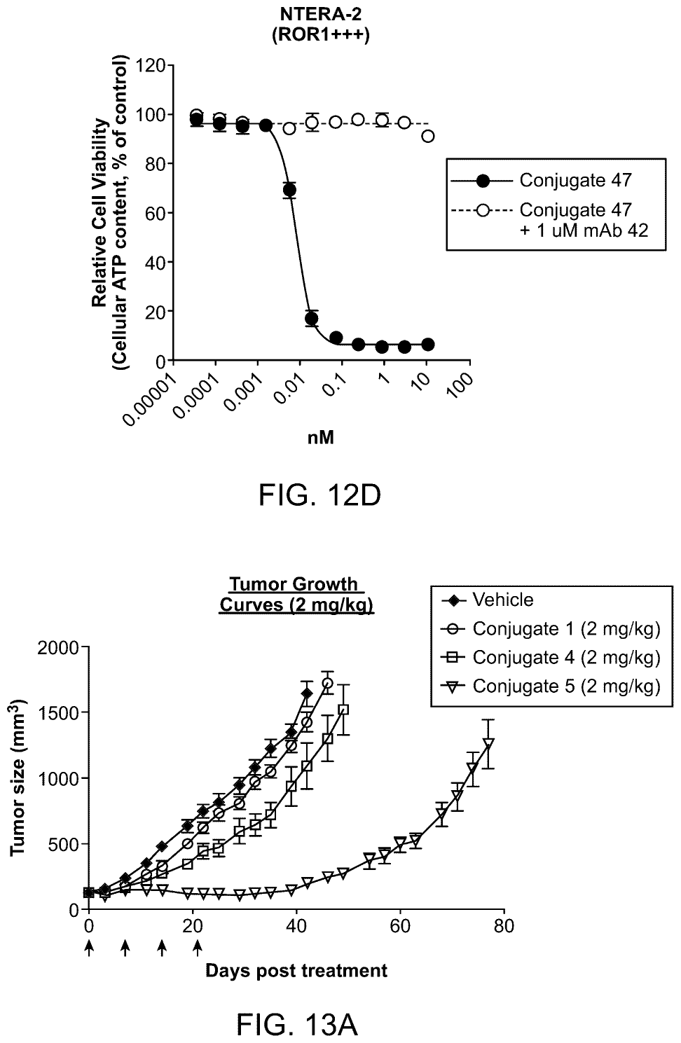

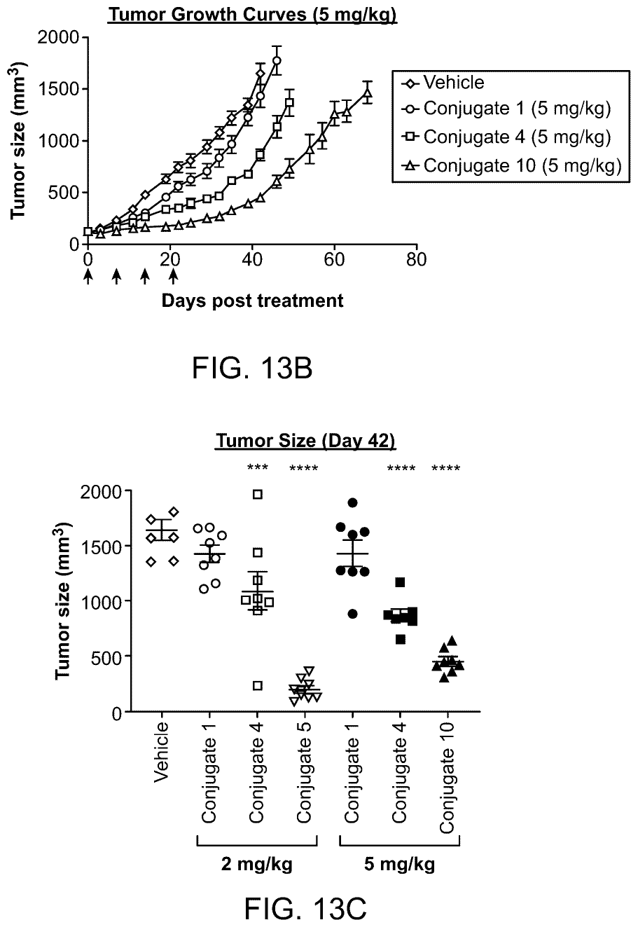

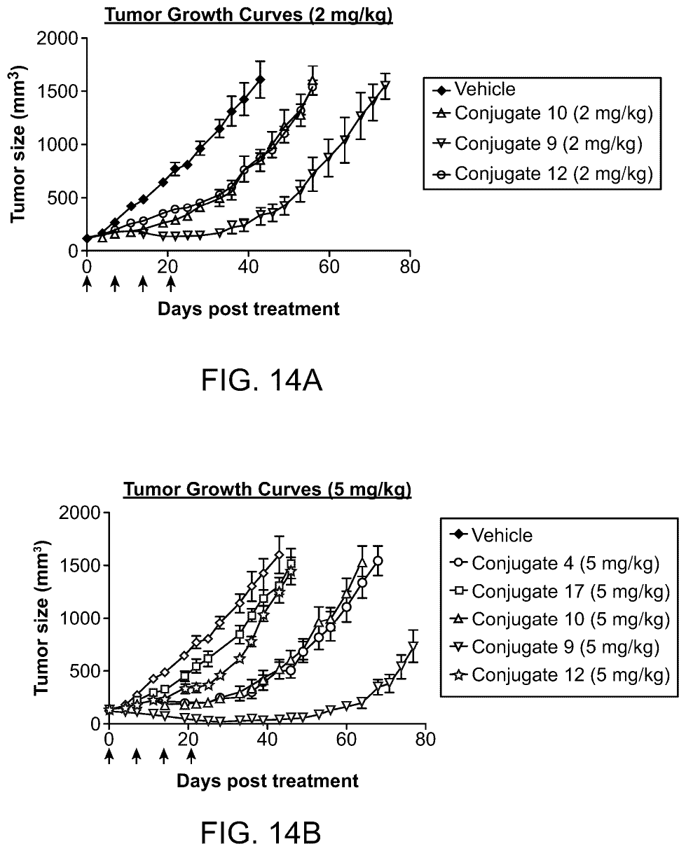

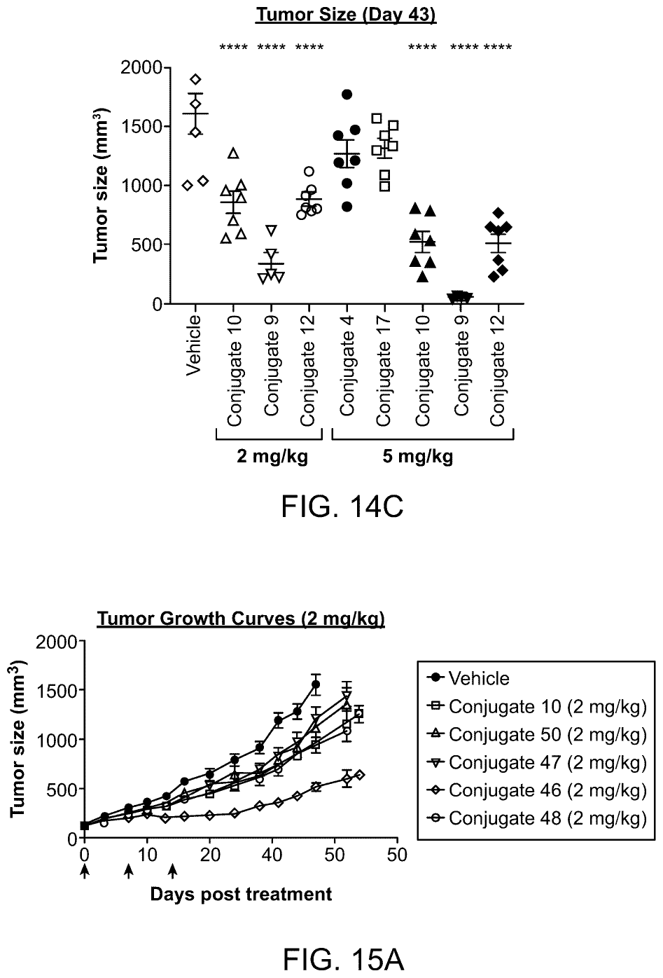

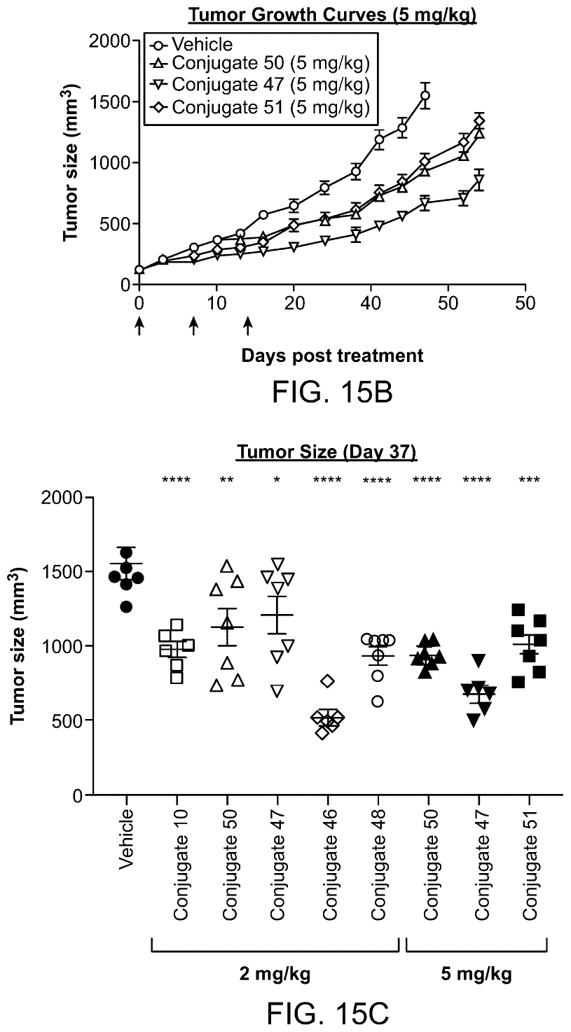

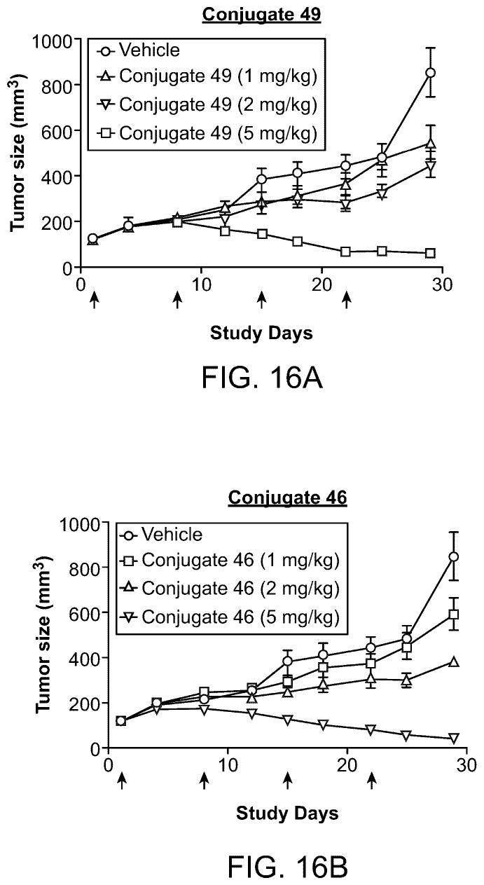

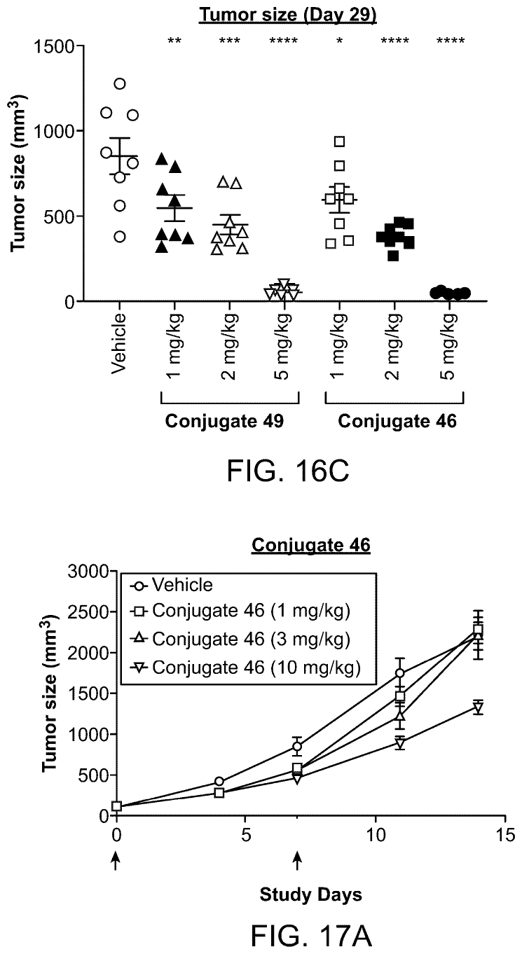

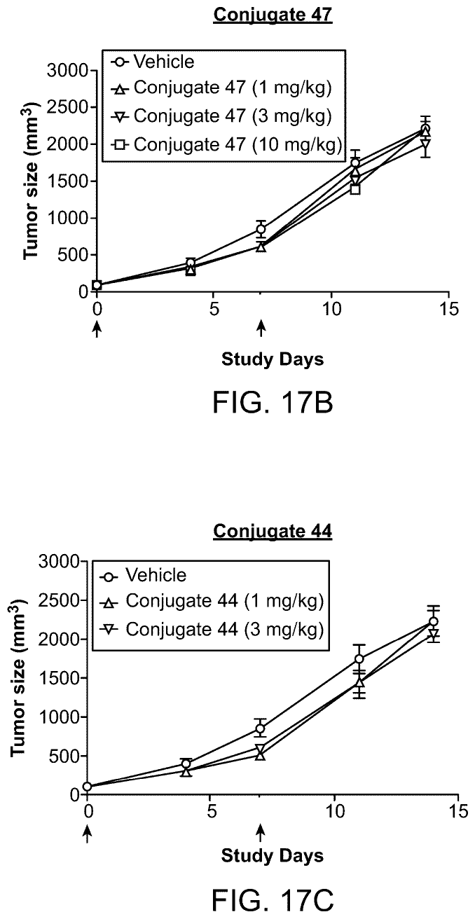

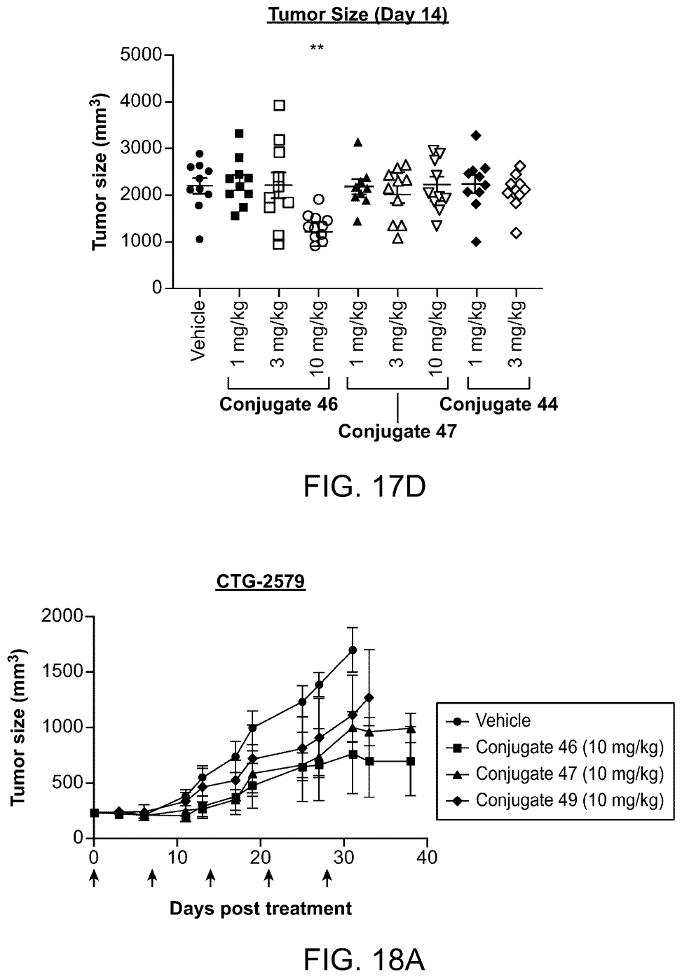

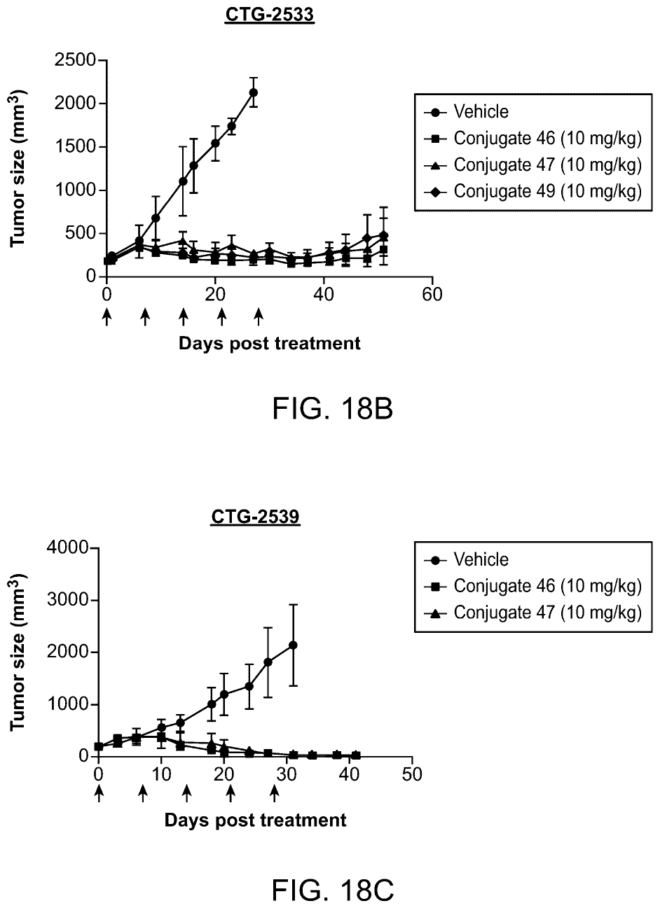

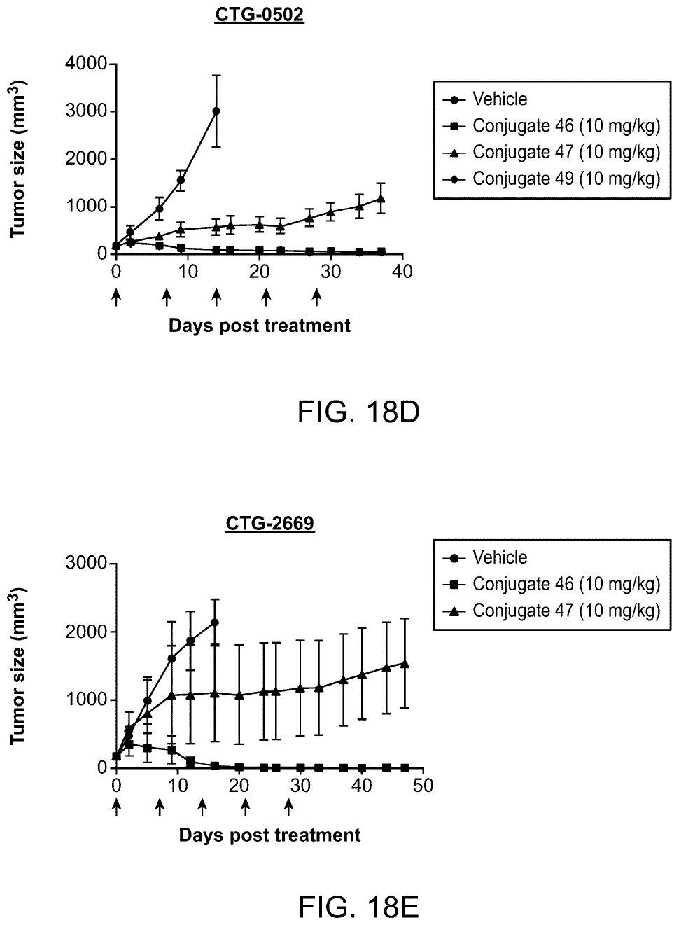

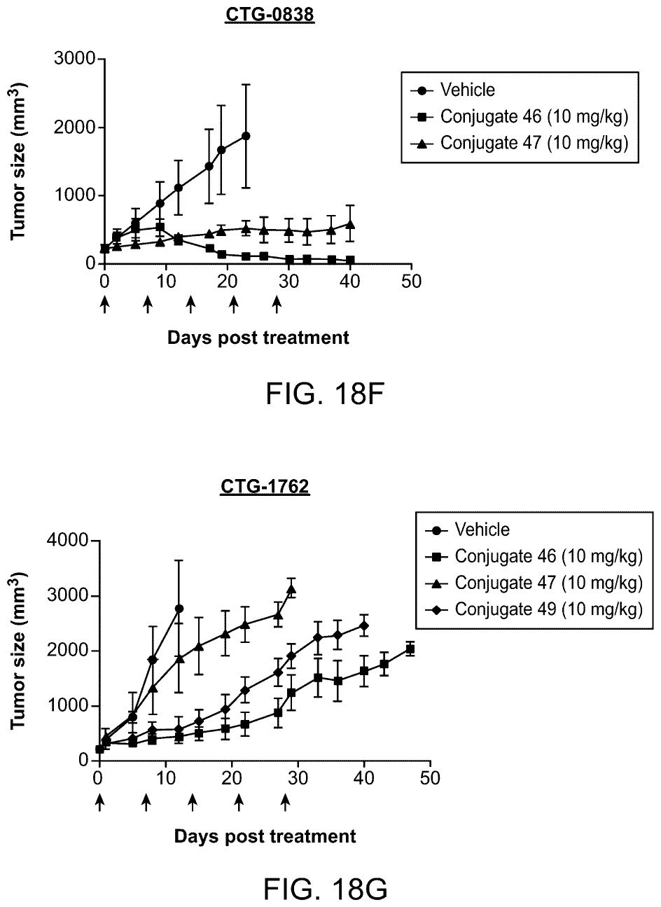

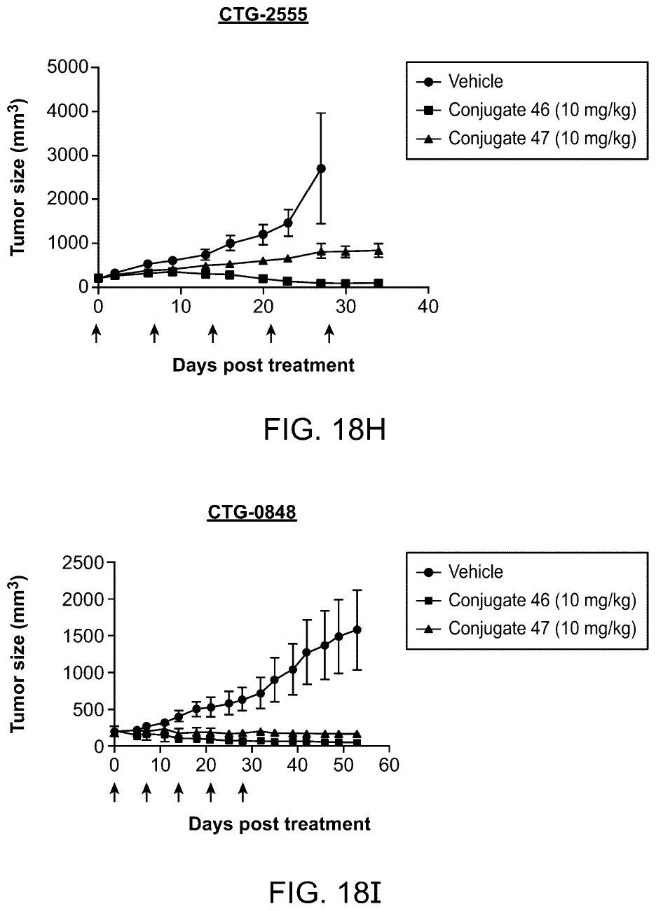

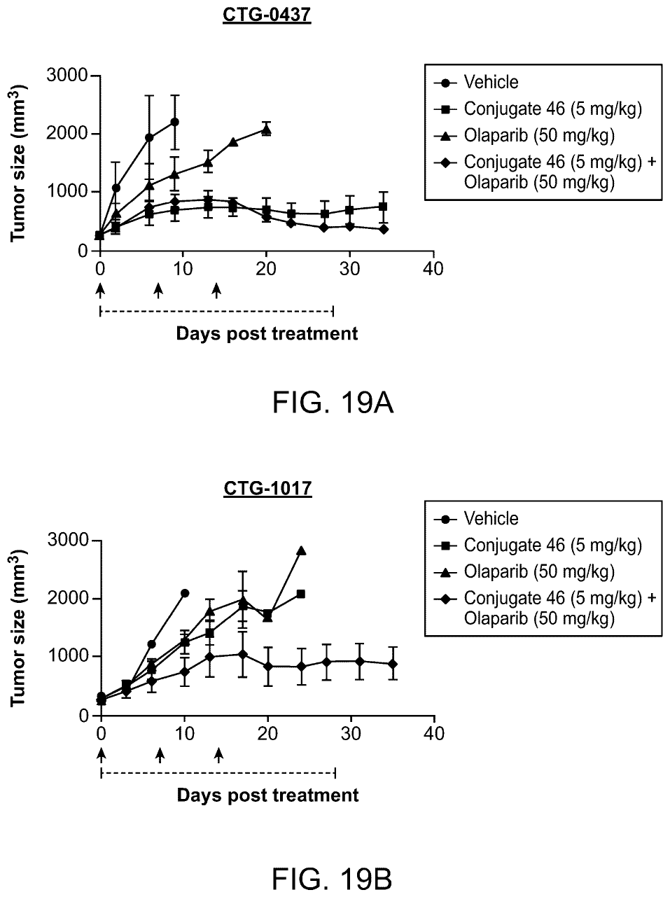

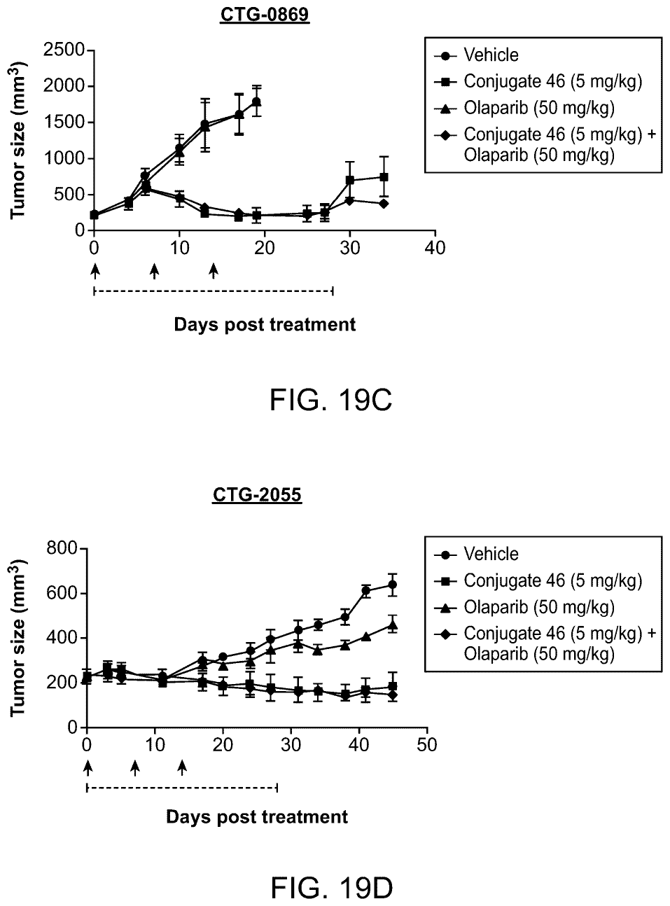

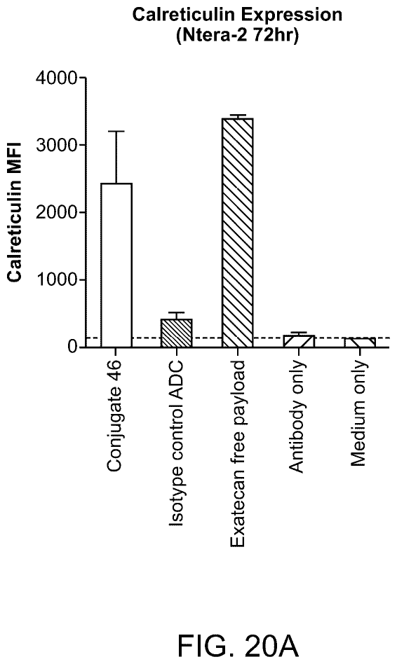

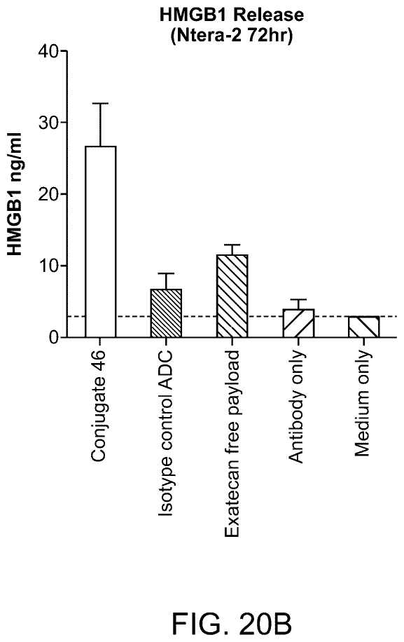

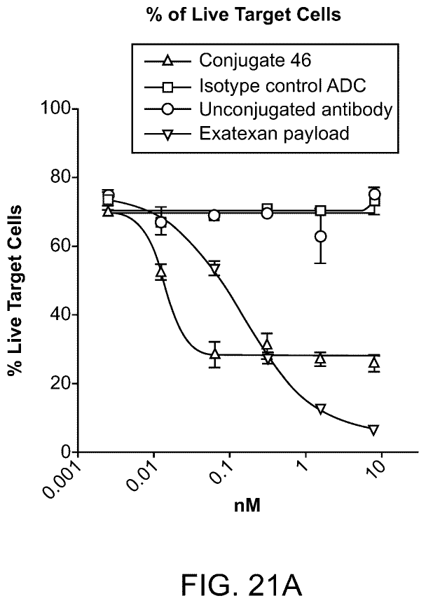

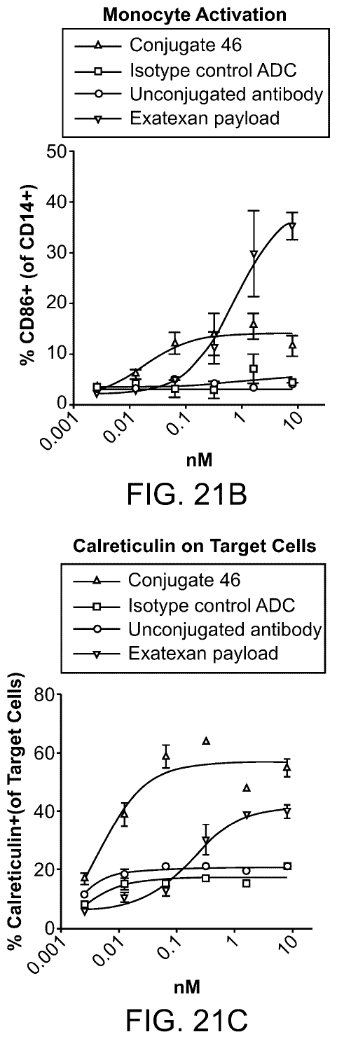

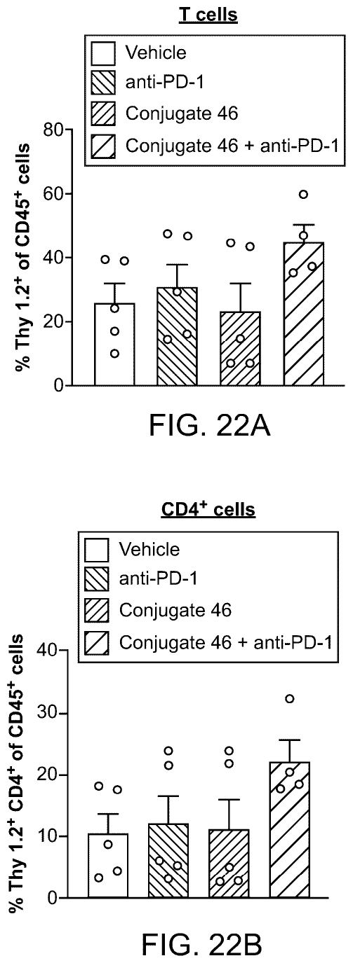

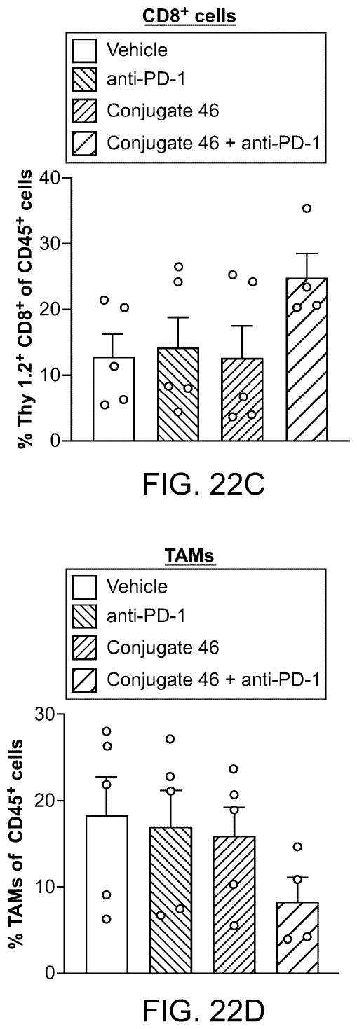

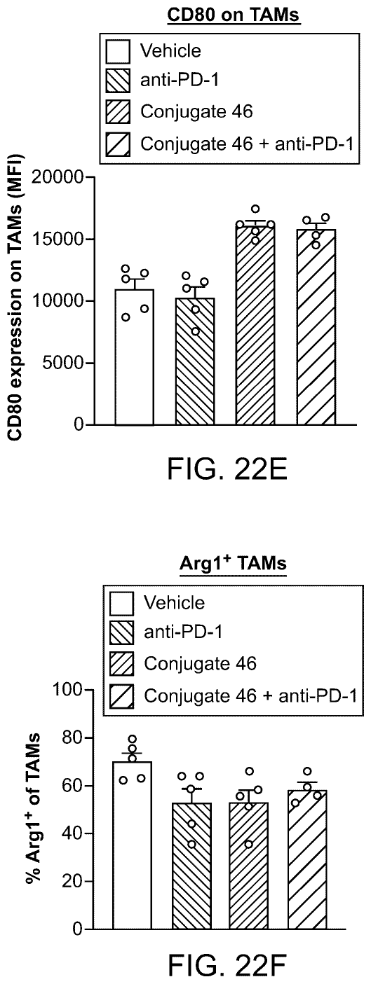

*The C-terminus of CDR-H1, when numbered using the Kabat numbering convention, varies between H32 and H34, depending on the length of the CDR, as illustrated in FIG. 1.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)2” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO: 1034. In some embodiments, the linker is SEQ ID NO: 1035. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 1027, or a portion thereof. SEQ ID NO: 1027 provides the sequence of C_{H1}, C_{H2}, and C_{H3} of the human IgG1 constant region.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec−1), as used herein, refers to the dissociation rate constant of a particular antibody-antigen interaction. This value is also referred to as the k_{off} value.

The term “kₐ” (M−1×sec−1), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M−1), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen (e.g., receptor tyrosine kinase orphan receptor 1, or ROR1). In one exemplary assay, ROR1 is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind the antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to variants of ROR1 with different point-mutations.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution of an amino acid with a chemically or functionally similar amino acid. Conservative substitution tables providing similar amino acids are well known in the art. Polypeptide sequences having such substitutions are known as “conservatively modified variants.” Such conservatively modified variants are in addition to and do not exclude polymorphic variants, interspecies homologs, and alleles. By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |
| Alcohol Group-Containing Residues | S and T |
| Aliphatic Residues | I, L, V, and M |
| Cycloalkenyl-associated Residues | F, H, W, and Y |
| Hydrophobic Residues | A, C, F, G, H, I, L, M, |
| | R, T, V, W, and Y |
| Negatively Charged Residues | D and E |
| Polar Residues | C, D, E, H, K, N, Q, R, |
| | S, and T |
| Positively Charged Residues | H, K, and R |
| Small Residues | A, C, D, G, N, P, S, T, |
| | and V |
| Very Small Residues | A, G, and S |
| Residues Involved | A, C, D, E, G, H, K, N, |
| in Turn Formation | Q, R, S, P, and T |
| Flexible Residues | Q, T, K, S, G, P, D, E, |
| | and R |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that
are considered conservative substitutions
for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An antibody generated by making one or more conservative substitutions of amino acid residues in a parent antibody is referred to as a “conservatively modified variant.”

The term “payload” refers to a molecular moiety that can be conjugated to an antibody. In particular embodiments, payloads are selected from the group consisting of therapeutic moieties and labelling moieties.

“Triple negative breast cancer” (TNBC) refers to a breast cancer characterized as estrogen receptor-negative, progesterone receptor-negative and human epidermal growth factor receptor-2-negative (HER2-negative). The TNBC can be BRCA1/2 wildtype or BRCA1/2 mutated. The determination of negative status of the estrogen, progesterone, and Her2/neu expression is readily determined by one of skill in the art, e.g., in accordance with the current accepted guidelines. For example, guidelines set forth by the American Society of Clinical Oncology (ASCO) and the College of American Pathologists (CAP) are widely accepted. The ASCO/CAP recommends testing by immunohistochemistry (IHC) or in situ hybridization (ISH) techniques. Further, a cancer is Her2 negative if a single test (or all tests) performed on a tumor specimen show: (a) IHC negative, IHC 1+ or IHC 0, or (b) ISH negative using single-probe ISH or dual-probe ISH. One of skill in the art would recognize that the triple negative cancer described herein does not include any cancer having an apparent histopathologic discordance as observed by the pathologist. Wolff, A C et al. J Clin Oncol. 2013 Nov. 1:31 (31): 3997-4013. Cancer is ER-negative or PR-negative if <1% of tumor cell nuclei are immunoreactive in the presence of evidence that the sample can express ER or PR (positive intrinsic controls are seen).

“PARP inhibitor-resistant” refers to the reduced effectiveness of PARP inhibitors in treating, curing, or improving triple negative breast cancer in a subject. In certain embodiments, the PARP inhibitor-resistance develops with prolonged exposure to one or more PARP inhibitors.

As used herein, the term “therapeutically effective amount” or “effective amount” refers to an amount of an antibody, antibody conjugate, or composition that when administered to a subject is effective to treat a disease or disorder. In some embodiments, a therapeutically effective amount or effective amount refers to an amount of an antibody, antibody conjugate, or composition that when administered to a subject is effective to prevent or ameliorate a disease or the progression of the disease, or result in amelioration of symptoms. A “therapeutically effective amount” can vary depending on, inter alia, the compound, the disease or disorder and its severity, and the age, weight, etc., of the subject to be treated.

As used herein, the term “inhibits growth” (e.g., referring to cells, such as tumor cells) is intended to include any measurable decrease in cell growth (e.g., tumor cell growth) when contacted with a ROR1 antibody or antibody conjugate described herein, as compared to the growth of the same cells not in contact with a ROR1 antibody. In some embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to antibody internalization, apoptosis, necrosis, and/or effector function-mediated activity.

In some chemical structures illustrated herein, certain substituents, chemical groups, and atoms are depicted with a curvy/wavy line

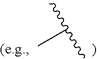

that intersects a bond or bonds to indicate the atom through which the substituents, chemical groups, and atoms are bonded. For example, in some structures, such as but not limited to

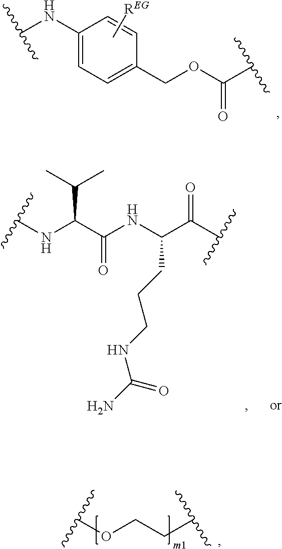

this curvy/wavy line indicates the atoms in the backbone of a conjugate or linker-payload structure to which the illustrated chemical entity is bonded. In some structures, such as but not limited to

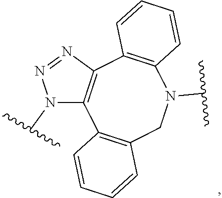 [SMILES: *N1Cc2ccccc2-c2c(nnn2*)-c2ccccc21]

this curvy/wavy line indicates the atoms in the antibody or antibody fragment as well as the atoms in the backbone of a conjugate or linker-payload structure to which the illustrated chemical entity is bonded.

Spiro compounds depicted with overlapping rings indicate that the rings can bond at any vertex. For instance, in the spiro group

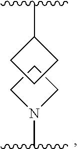

the two rings can bond at any of the three available vertex atoms in either ring.

When referring to the compounds provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

“Alkoxy” and “alkoxyl,” refer to the group —OR″ where R″ is alkyl or cycloalkyl. Alkoxy groups include, in certain embodiments, methoxy, ethoxy, n-propoxy, isopropoxy, n-butoxy, tert-butoxy, sec-butoxy, n-pentoxy, n-hexoxy, 1,2-dimethylbutoxy, and the like.

The term “alkoxyamine,” as used herein, refers to the group -alkylene-O—NH₂, wherein alkylene is as defined herein. In some embodiments, alkoxyamine groups can react with aldehydes to form oxime residues. Examples of alkoxyamine groups include-CH₂CH₂—O—NH₂, —CH₂—O—NH₂, and —O—NH₂.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms (i.e., C₁ to C₁₀ alkyl). In certain embodiments, the alkyl is a lower alkyl, for example, C1-6alkyl, and the like. In certain embodiments, the alkyl group is selected from the group consisting of methyl, ethyl, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl. In certain embodiments, “substituted alkyl” refers to an alkyl substituted with, for example, one, two, or three groups independently selected from a halogen (e.g., fluoro (F), chloro (Cl), bromo (Br), or iodo (I)), alkyl, —CN, —NO₂, amido, —C(O)—, —C(S)—, ester, carbamate, alkenyl, alkynyl, cycloalkyl, heterocycloalkyl, dialkylamino, haloalkyl, hydroxyl, amino, alkylamino, and alkoxy. In some embodiments, alkyl is unsubstituted.

The term “alkylene,” as used herein, unless otherwise specified, refers to a divalent alkyl group, as defined herein. “Substituted alkylene” refers to an alkylene group substituted as described herein for alkyl. In some embodiments, alkylene is unsubstituted.

“Alkenyl” refers to an olefinically unsaturated hydrocarbon group, in certain embodiments, having up to about eleven carbon atoms or from two to six carbon atoms (e.g., “lower alkenyl”), which can be straight-chained or branched, and having at least one or from one to two sites of olefinic unsaturation. “Substituted alkenyl” refers to an alkenyl group substituted as described herein for alkyl.

“Alkenylene” refers to a divalent alkenyl as defined herein. Lower alkenylene is, for example, C₂-C₆-alkenylene.

“Alkynyl” refers to acetylenically unsaturated hydrocarbon groups, in certain embodiments, having up to about eleven carbon atoms or from two to six carbon atoms (e.g., “lower alkynyl”), which can be straight-chained or branched, and having at least one or from one to two sites of acetylenic unsaturation. Non-limiting examples of alkynyl groups include acetylene (—C≡CH), propargyl (—CH₂C≡CH), and the like. “Substituted alkynyl” refers to an alkynyl group substituted as described herein for alkyl.

“Alkynylene” refers to a divalent alkynyl as defined herein. Lower alkynylene is, for example, C₂-C₆-alkynylene.

“Amino” refers to —NH₂.

The term “alkylamino,” as used herein, and unless otherwise specified, refers to the group —NHR″ where R″ is, for example, C₁₋₁₀alkyl, C₂₋₁₀ alkenyl, C₂₋₁₀ alkynyl, C₃₋₁₂ carbocycle, 3- to 12-membered heterocycle, C₁₋₁₀ haloalkyl, and the like as defined herein. In certain embodiments, alkylamino is C₁₋₆alkylamino.

The term “dialkylamino,” as used herein, and unless otherwise specified, refers to the group-NR″R″ where each R″ is independently _{C1-10}alkyl, as defined herein. In certain embodiments, dialkylamino is, for example, di-C₁₋₆alkylamino, C₂₋₁₀ alkenyl, C₂₋₁₀ alkynyl, C₃₋₁₂ carbocycle, 3- to 12-membered heterocycle, C₁₋₁₀ haloalkyl, and the like.

The term “aryl,” as used herein, and unless otherwise specified, refers to phenyl, biphenyl, or naphthyl. The term includes both substituted and unsubstituted moieties. An aryl group can be substituted with any described moiety including, but not limited to, one or more moieties (e.g., in some embodiments one, two, or three moieties) selected from the group consisting of halogen (e.g., fluoro (F), chloro (Cl), bromo (Br), or iodo (I)), alkyl, haloalkyl, hydroxyl, amino, alkylamino, arylamino, alkoxy, aryloxy, nitro, cyano, sulfonic acid, sulfate, phosphonic acid, phosphate, and phosphonate, wherein each moiety is independently either unprotected, or protected as necessary, as would be appreciated by those skilled in the art (see, e.g., Greene, et al., Protective Groups in Organic Synthesis, John Wiley and Sons, Second Edition, 1991); and wherein the aryl in the arylamino and aryloxy substituents are not further substituted.

The term “arylamino,” as used herein, and unless otherwise specified, refers to an —NR′R″ group where R′ is hydrogen or C₁-C₆-alkyl; and R″ is aryl, as defined herein.

The term “arylene,” as used herein, and unless otherwise specified, refers to a divalent aryl group, as defined herein.

The term “aryloxy,” as used herein, and unless otherwise specified, refers to an —OR group where R is aryl, as defined herein.

“Alkarylene” refers to an arylene group, as defined herein, wherein the aryl ring is substituted with one or two alkyl groups. “Substituted alkarylene” refers to an alkarylene, as defined herein, where the arylene group is further substituted, as defined herein for aryl.

“Aralkylene” refers to a —CH₂-arylene-, -arylene-CH₂—, or —CH₂-arylene-CH₂— group, where arylene is as defined herein. “Substituted aralkylene” refers to an aralkylene, as defined herein, where the aralkylene group is substituted, as defined herein for aryl.

“Carboxyl” or “carboxy” refers to —C(O)OH or —COOH.

The term “cycloalkyl” as used herein, unless otherwise specified, refers to a saturated cyclic hydrocarbon. In certain embodiments, the cycloalkyl group may be saturated, and/or bridged, and/or non-bridged, and/or a fused bicyclic group and/or a spirocyclic bicyclic group. In certain embodiments, the cycloalkyl group includes three to ten carbon atoms (i.e., C₃ to C₁₀ cycloalkyl). In some embodiments, the cycloalkyl has from three to fifteen carbons (C₃₋₁₅), from three to ten carbons (C₃₋₁₀), from three to seven carbons (C₃₋₇), or from three to six carbons (C₃-C₆) (i.e., “lower cycloalkyl”). In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl. Exemplary “cycloalkyl” or “carbocycles” include cyclopentyl, cyclohexyl, cyclohexenyl, adamantyl, phenyl, indanyl, and naphthyl. “Cycloalkyl” or “carbocycle” includes 3- to 10-membered monocyclic rings, 6- to 12-membered bicyclic rings, and 6- to 12-membered bridged rings. Each ring of a bicyclic cycloalkyl or carbocycle may be selected from saturated, unsaturated, and aromatic rings. A bicyclic cycloalkyl or carbocycle includes any combination of saturated, unsaturated and aromatic bicyclic rings, as valence permits. A bicyclic cycloalkyl or carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Non-limiting examples of bridged bicyclic cycloalkyl or carbocycle groups include, but are not limited to, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, and 2-oxabicyclo[2.2.2]octyl. Non-limiting examples of spirocyclic cycloalkyl or carbocycle groups include, but are not limited to, spiro[3.3]heptyl, spiro[3.4]octyl, spiro[3.5]nonyl, spiro[3.6]decyl, spiro[4.4]nonyl, spiro[4.5]decyl, spiro[5.5]undecyl, spiro[5.6]dodecyl, and spiro[5.7]tridecyl.

The term “bicyclic ring system” includes 6-12 (e.g., 8-12 or 9-, 10-, or 11-) membered structures that form two rings, wherein the two rings have at least one atom in common (e.g., two atoms in common). Bicyclic rings can be fused, bridged, or spirocyclic. Bicyclic ring systems include bicycloaliphatics (e.g., bicycloalkyl or bicycloalkenyl), bicycloheteroaliphatics, bicyclic aryls, and bicyclic heteroaryls.

The term “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[1.1.1]pentyl, bicyclo[2.1.1]hexyl, bicyclo[2.1.1]hexyl, bicyclo[3.1.1]heptyl, bicyclo[2.2.1]heptyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

The term “spiro bicyclic ring system” refers to a bicyclic heterocyclicalipahtic ring system or bicyclic cycloaliphatic ring system in which 2 or 3 rings are linked together by one common atom. Spiro compounds depicted with overlapping rings indicate that the rings can bond at any vertex. For instance, in the spiro group

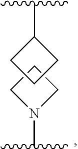

the two rings can bond at any of the three available vertex atoms in either ring.

The term “cycloalkylene,” as used herein refers to a divalent cycloalkyl group, as defined herein. In certain embodiments, the cycloalkylene group is cyclopropylene

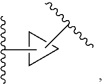

cyclobutylene

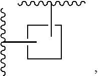

cyclopentylene

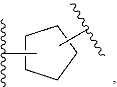

cyclohexylene

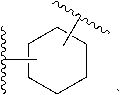

cycloheptylene

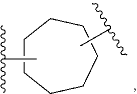

and the like. Lower cycloalkylene refers to a C₃-C₆-cycloalkylene.

The term “cycloalkylalkyl,” as used herein, unless otherwise specified, refers to an alkyl group, as defined herein, substituted with one or two cycloalkyl, as defined herein.

The term “ester,” as used herein, refers to —C(O)OR or —COOR where R is alkyl, as defined herein.

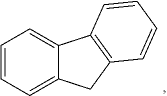 [SMILES: c1ccc2c(c1)Cc1ccccc1-2]

The term “fluorene” as used herein refers to wherein any one or more carbons bearing one or more hydrogens can be substituted with a chemical functional group as described herein.

The term “haloalkyl” refers to an alkyl group, as defined herein, substituted with one or more halogen atoms (e.g., in some embodiments one, two, three, four, or five) which are independently selected.

The term “heteroalkyl” refers to an alkyl, as defined herein, in which one or more carbon atoms are replaced by heteroatoms. As used herein, “heteroalkenyl” refers to an alkenyl, as defined herein, in which one or more carbon atoms are replaced by heteroatoms. As used herein, “heteroalkynyl” refers to an alkynyl, as defined herein, in which one or more carbon atoms are replaced by heteroatoms. Suitable heteroatoms include, but are not limited to, nitrogen (N), oxygen (O), and sulfur(S) atoms. Heteroalkyl, heteroalkenyl, and heteroalkynyl are optionally substituted. Examples of heteroalkyl moieties include, but are not limited to, aminoalkyl, sulfonylalkyl, and sulfinylalkyl. Examples of heteroalkyl moieties also include, but are not limited to, methylamino, methylsulfonyl, and methylsulfinyl. “Substituted heteroalkyl” refers to heteroalkyl substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heteroalkyl group may comprise one, two, three, or four heteroatoms. Those of skill in the art will recognize that a 4-membered heteroalkyl may generally comprise one or two heteroatoms, a 5- or 6-membered heteroalkyl may generally comprise one, two, or three heteroatoms, and a 7- to 10-membered heteroalkyl may generally comprise one, two, three, or four heteroatoms.

The term “heteroalkylene,” as used herein, refers to a divalent heteroalkyl, as defined herein. “Substituted heteroalkylene” refers to a divalent heteroalkyl, as defined herein, substituted as described for heteroalkyl.

The term “heterocycloalkyl” refers to a monovalent, monocyclic, or multicyclic non-aromatic ring system, wherein one or more of the ring atoms are heteroatoms independently selected from oxygen (O), sulfur(S), and nitrogen (N) (e.g., where the nitrogen or sulfur atoms may be optionally oxidized, and the nitrogen atoms may be optionally quaternized) and the remaining ring atoms of the non-aromatic ring are carbon atoms. In certain embodiments, heterocycloalkyl is a monovalent, monocyclic, or multicyclic fully-saturated ring system. In certain embodiments, the heterocycloalkyl group has from three to twenty, from three to fifteen, from three to ten, from three to eight, from four to seven, from four to eleven, or from five to six ring atoms. The heterocycloalkyl may be attached to a core structure at any heteroatom or carbon atom which results in the creation of a stable compound. In certain embodiments, the heterocycloalkyl is a monocyclic, bicyclic, tricyclic, or tetracyclic ring system, which may include a fused, and/or bridged bicyclic group, and/or a spirocyclic bicyclic group ring system and in which the nitrogen or sulfur atoms may be optionally oxidized, and/or the nitrogen atoms may be optionally quaternized. In some embodiments, heterocycloalkyl radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocycloalkyl may also be optionally substituted as described herein. In certain embodiments, heterocycloalkyl is substituted with one, two, or three groups independently selected from halogen (e.g., fluoro (F), chloro (Cl), bromo (Br), or iodo (I)), alkyl, haloalkyl, hydroxyl, amino, alkylamino, and alkoxy. In some embodiments, a heterocycloalkyl group may comprise one, two, three, or four heteroatoms. Those of skill in the art will recognize that a 4-membered heterocycloalkyl may generally comprise one or two heteroatoms, a 5- or 6-membered heterocycloalkyl may generally comprise one, two, or three heteroatoms, and a 7- to 10-membered heterocycloalkyl may generally comprise one, two, three, or four heteroatoms. In some embodiments, “heterocycloalkyl” or “heterocycle” radicals include, but are not limited to, 2,5-diazabicyclo[2.2.2]octanyl, decahydroisoquinolinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. Non-limiting examples of bridged heterocycloalkyl or heterocycle groups include, but are not limited to, 6-azabicyclo[3.1.1]heptyl, 6-azabicyclo[3.1.1]heptyl, 1-azabicyclo[2.2.1]heptyl, 2-azabicyclo[2.2.1]heptyl, 7-azabicyclo[2.2.1]heptyl, 1-azabicyclo[2.2.2]octyl, 3-azabicyclo[3.2.1]octyl, and 2-oxabicyclo[3.1.1]heptyl, 2,6-dioxa-tricyclo[3.3.1.03,7]nonyl. Non-limiting examples of spirocyclic heterocycloalkyl or heterocycle groups include, but are not limited to, 2,8-diazaspiro[4.5]decyl; 2,7-diazaspiro[3.5]nonyl; 3,9-diazaspiro[5.5]undecyl; 3-azaspiro[5.5]undecyl; 2-oxa-6-azaspiro[3.4]octyl; 2-oxa-9-azaspiro[5.5]undecyl; 3-oxa-9-azaspiro[5.5]undecyl; 7-azaspiro[3.5]nonyl; 2-azaspiro[3.5]nonyl; 7-oxaspiro[3.5]nonyl; and, 2-oxaspiro[3.5]nonyl.

“Heterocycloalkylene” refers to a divalent heterocycloalkyl as defined herein.

The term “heteroaryl” refers to a monovalent, monocyclic aromatic group and/or multicyclic aromatic group, wherein at least one aromatic ring contains one or more heteroatoms independently selected from oxygen, sulfur, and nitrogen within the ring. Each ring of a heteroaryl group can contain one or two oxygen atoms, one or two sulfur atoms, and/or one to four nitrogen atoms, provided that the total number of heteroatoms in each ring is four or less and each ring contains at least one carbon atom. In certain embodiments, the heteroaryl has from five to twenty, from five to fifteen, or from five to ten ring atoms. A heteroaryl may be attached to the rest of the molecule via a nitrogen or a carbon atom. In some embodiments, monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, triazolyl, thiadiazolyl, thiazolyl, thienyl, tetrazolyl, and triazinyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein. “Substituted heteroaryl” is a heteroaryl substituted as defined for aryl.

The term “heteroarylene” refers to a divalent heteroaryl group, as defined herein. “Substituted heteroarylene” is a heteroarylene substituted as defined for aryl.

The term “protecting group,” as used herein, and unless otherwise specified, refers to a group that is added to an oxygen, nitrogen, or phosphorus atom to prevent further reaction at the (protected) oxygen, nitrogen, or phosphorus, or for other purposes. A wide variety of oxygen and nitrogen protecting groups are known to those skilled in the art of organic synthesis (see, e.g., Greene, et al., Protective Groups in Organic Synthesis, John Wiley and Sons, Fourth Edition, 2006, which is incorporated herein by reference in its entirety).

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, glutamic, hydroxynaphthoic, salicylic, stearic, cyclohexylsulfamic, quinic, and muconic acids, and the like; or (2) salts formed when an acidic proton present in the parent compound either (a) is replaced by a metal ion, for example, an alkali metal ion, an alkaline earth ion, or an aluminum ion, or alkali metal or alkaline earth metal hydroxides, such as sodium, potassium, calcium, magnesium, aluminum, lithium, zinc, and barium hydroxide, or ammonia; or (b) coordinates with an organic base, such as aliphatic, alicyclic, or aromatic organic amines, including, without limitation, ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example and without limitation, sodium, potassium, calcium, magnesium, ammonium, and tetraalkylammonium salts, and the like, and when the compound contains a basic functionality, salts of non-toxic organic or inorganic acids, such as hydrohalides, for example, hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2 hydroxyethanesulfonate, benzenesulfonate (besylate), 4-chlorobenzenesulfonate, 2 naphthalenesulfonate, 4-toluenesulfonate, camphorate, camphorsulfonate, 4 methylbicyclo[2.2.2]-oct-2-ene-1-carboxylate, glucoheptonate, 3-phenylpropionate, trimethylacetate, tert-butylacetate, lauryl sulfate, gluconate, glutamate, hydroxynaphthoate, salicylate, stearate, cyclohexylsulfamate, quinate, muconate, and the like.

The term “substantially free of” or “substantially in the absence of” with respect to a composition refers to a composition that includes at least 85% or 90% by weight, in certain embodiments 95%, 98%, 99%, or 100% by weight; or in certain embodiments, 95%, 98%, 99%, or 100% of the designated enantiomer or diastereomer of a compound. In certain embodiments, in the methods and compounds provided herein, the compounds are substantially free of one of two enantiomers. In certain embodiments, in the methods and compounds provided herein, the compounds are substantially free of one of two diastereomers. In certain embodiments, in the methods and compounds provided herein, the compounds are substantially free of enantiomers (i.e., the compounds are not a racemic or 50:50 mixture of compounds).

Similarly, the term “isolated” with respect to a composition refers to a composition that includes at least 85%, 90%, 95%, 98%, or 99% to 100% by weight, of the compound, the remainder comprising other chemical species, enantiomers, or diastereomers.

“Solvate” refers to a compound provided herein, or a salt thereof, that further includes a stoichiometric or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as hydrogen (H), the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium (D) enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise deuterium (D) at one or more positions where hydrogen (H) atoms are present, and wherein the deuterium composition of the atom or atoms is other than the natural isotopic composition.

Also as used herein, “alkyl,” “alkylene,” “alkylamino,” “dialkylamino,” “cycloalkyl,” “aryl,” “arylene,” “alkoxy,” “amino,” “carboxyl,” “heterocycloalkyl,” “heteroaryl,” “heteroarylene,” “carboxyl,” and “amino acid” groups optionally comprise carbon-13 (¹³C) at an amount other than the natural isotopic composition.

The term “macromolecule” or “macromolecular moiety” refers to a protein, peptide, antibody, nucleic acid, carbohydrate, or other large molecule composed of polymerized monomers. They include peptides of two or more residues, or ten or more residues. In certain embodiments, a macromolecule is at least 1000 Da in mass. In certain embodiments, a macromolecule has at least 1000 atoms. In certain embodiments, a macromolecule can be modified. For instance, a protein, peptide, or antibody can be modified with one or more carbohydrates and/or small molecule therapeutic compounds.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V), and the less common pyrrolysine and selenocysteine. Natural amino acids also include citrulline. Naturally encoded amino acids include post-translational variants of the twenty-two naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids, and acylated amino acids. The term “amino acid” also includes non-natural (or unnatural) or synthetic α-, β-, γ-, or δ-amino acids, and includes, but is not limited to, amino acids found in proteins, i.e., glycine, alanine, valine, leucine, isoleucine, methionine, phenylalanine, tryptophan, proline, serine, threonine, cysteine, tyrosine, asparagine, glutamine, aspartate, glutamate, lysine, arginine, and histidine. In certain embodiments, the amino acid is in the L-configuration. In certain embodiments, the amino acid is in the D-configuration. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β serinyl, β-threoninyl, β-cysteinyl, β-tyrosinyl, β-asparaginyl, β-glutaminyl, β-aspartoyl, β glutaroyl, β-lysinyl, β-argininyl, or β-histidinyl. Unnatural amino acids are not proteinogenic amino acids, or post-translationally modified variants thereof. In particular, the term unnatural amino acid refers to an amino acid that is not one of the twenty common amino acids or pyrrolysine or selenocysteine, or post-translationally modified variants thereof.

The term “conjugate” or “antibody conjugate” refers to a compound or drug moiety described herein linked to one or more macromolecular moieties. The macromolecular moiety is as defined herein or is any macromolecule deemed suitable to the person of skill in the art. The compound or drug moiety can be any compound or drug moiety described herein. The compound or drug moiety can be directly linked to the macromolecular moiety via a covalent bond, or the compound or drug moiety can be linked to the macromolecular moiety indirectly via a linker. Typically, the linker is covalently bonded to the macromolecular moiety and also covalently bonded to the compound or drug moiety.

“pAMF,” “pAMF residue,” or “pAMF mutation” refers to a variant phenylalanine residue (i.e., para-azidomethyl-L-phenylalanine) added or substituted into a polypeptide.

The term “linker” refers to a molecular moiety that is capable of forming at least two covalent bonds. Typically, a linker is capable of forming at least one covalent bond to a macromolecular moiety and at least another covalent bond to a compound or drug moiety. In certain embodiments, a linker can form more than one covalent bond to a macromolecular moiety. In certain embodiments, a linker can form more than one covalent bond to a compound or drug moiety or can form covalent bonds to more than one compound or drug moiety. After a linker forms a bond to a macromolecular moiety, or a compound or drug moiety, or both, the remaining structure (i.e. the residue of the linker (“linker residue”) after one or more covalent bonds are formed) may still be referred to as a “linker” herein. The term “linker precursor” refers to a linker having one or more reactive groups capable of forming a covalent bond with a macromolecule, or compound or drug moiety, or both. A person of ordinary skill in the art, given the context of how the term linker is used, would understand whether “linker” means linker precursor with one reactive group, a linker precursor with more than one reactive groups, a linker residue which is covalently bonded to the macromolecule, a linker residue which is covalently bonded to a compound or drug moiety, and/or a linker residue which is covalently bonded to the macromolecule and is covalently bonded to a compound or drug moiety. In some embodiments, the linker is a cleavable linker. For example, a cleavable linker can be one that is released by a bio-labile or enzymatic function, which may or may not be engineered. In some embodiments, the linker is a non-cleavable linker. For example, a non-cleavable linker can be one that is released upon degradation of the macromolecular moiety.

As used herein, term “EC₅₀” refers to a dosage, concentration, or amount of a particular test compound that elicits a dose-dependent response at 50% of maximal expression of a particular response that is induced, provoked, or potentiated by the particular test compound.

As used herein, and unless otherwise specified, the term “IC₅₀” refers to an amount, concentration, or dosage of a particular test compound that achieves a 50% inhibition of a maximal response in an assay that measures such response.

As used herein, the terms “subject” and “patient” are used interchangeably. The terms “subject” and “subjects” refer to an animal, such as a mammal including a non-primate (e.g., a cow, pig, horse, cat, dog, rat, mouse, camel, avian, goat, and sheep) and a primate (e.g., a monkey, such as a cynomolgous monkey, a chimpanzee, and a human), and in certain embodiments, a human. In certain embodiments, the subject is a farm animal (e.g., a horse, a cow, a pig, etc.) or a pet (e.g., a dog or a cat). In certain embodiments, the subject is a human. In some embodiments, the subject has a disease that can be treated or diagnosed with an antibody or antibody conjugate provided herein. In some embodiments, the disease is gastric carcinoma, colorectal carcinoma, renal cell carcinoma, cervical carcinoma, non-small cell lung carcinoma, ovarian cancer, breast cancer, triple-negative breast cancer, endometrial cancer, prostate cancer, and/or a cancer of epithelial origin.

As used herein, the terms “therapeutic agent” and “therapeutic agents” refer to any agent(s) which can be used in the treatment or prevention of a disorder or one or more symptoms thereof. In certain embodiments, the term “therapeutic agent” includes an antibody or antibody conjugate provided herein. In certain embodiments, a therapeutic agent is an agent which is known to be useful for, or has been or is currently being used for the treatment or prevention of a disorder or one or more symptoms thereof.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder, or delaying or preventing recurrence of the disease or disorder. In yet another embodiment, “treating” or “treatment” includes the reduction or elimination of either the disease or disorder, or retarding the progression of the disease or disorder or of one or more symptoms of the disease or disorder, or reducing the severity of the disease or disorder or of one or more symptoms of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound, drug moiety, or conjugate provided herein. In certain other embodiments, the term “prophylactic agent” does not refer a compound, drug moiety, or conjugate provided herein. For example, a prophylactic agent is an agent which is known to be useful for, or has been or is currently being used to prevent or impede the onset, development, progression, and/or severity of a disorder.

As used herein, the phrase “prophylactically effective amount” refers to the amount of a therapy (e.g., prophylactic agent) which is sufficient to result in the prevention or reduction of the development, recurrence, or onset of one or more symptoms associated with a disorder or to enhance or improve the prophylactic effect(s) of another therapy (e.g., another prophylactic agent).

In some chemical structures illustrated herein, certain substituents, chemical groups, and atoms are depicted with a curvy/wavy/wiggly line

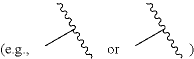

that intersects a bond or bonds to indicate the atom through which the substituents, chemical groups, and atoms are bonded. For example, in some structures, such as but not limited to,

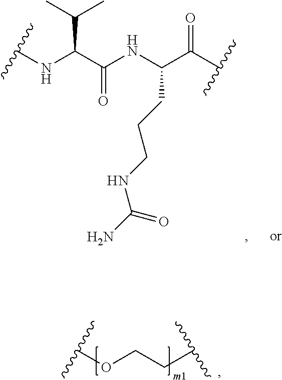

this curvy/wavy/wiggly line indicates the atoms in the backbone of a conjugate, compound, or drug moiety structure to which the illustrated chemical entity is bonded. In some structures, such as but not limited to

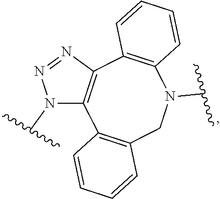 [SMILES: *N1Cc2ccccc2-c2c(nnn2*)-c2ccccc21]

this curvy/wavy/wiggly line indicates the atoms in the macromolecule as well as the atoms in the backbone of a conjugate, compound, or drug moiety structure to which the illustrated chemical entity is bonded.

As used herein, illustrations showing substituents bonded to a cyclic group (e.g., aromatic, heteroaromatic, fused ring, and saturated or unsaturated cycloalkyl or heterocycloalkyl) through a bond between ring atoms are meant to indicate, unless specified otherwise, that the cyclic group may be substituted with that substituent at any ring position in the cyclic group or on any ring in the fused ring group, according to techniques set forth herein or which are known in the field to which the instant disclosure pertains. For example, the group,

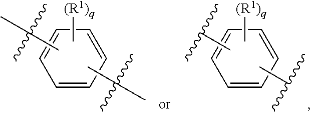

wherein subscript q is an integer from zero to four and in which the positions of substituent R¹ are described generically, i.e., not directly attached to any vertex of the bond line structure, i.e., specific ring carbon atom, includes the following, non-limiting examples of groups in which the substituent R¹ is bonded to a specific ring carbon atom:

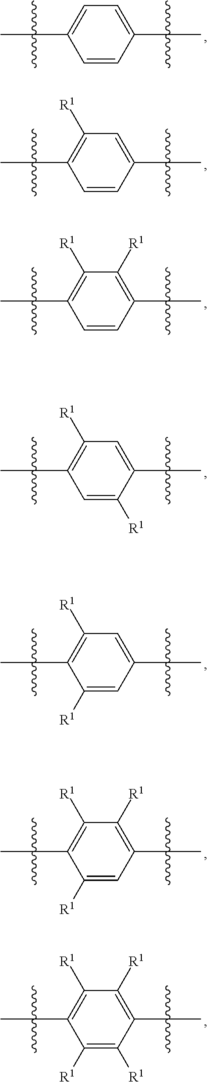

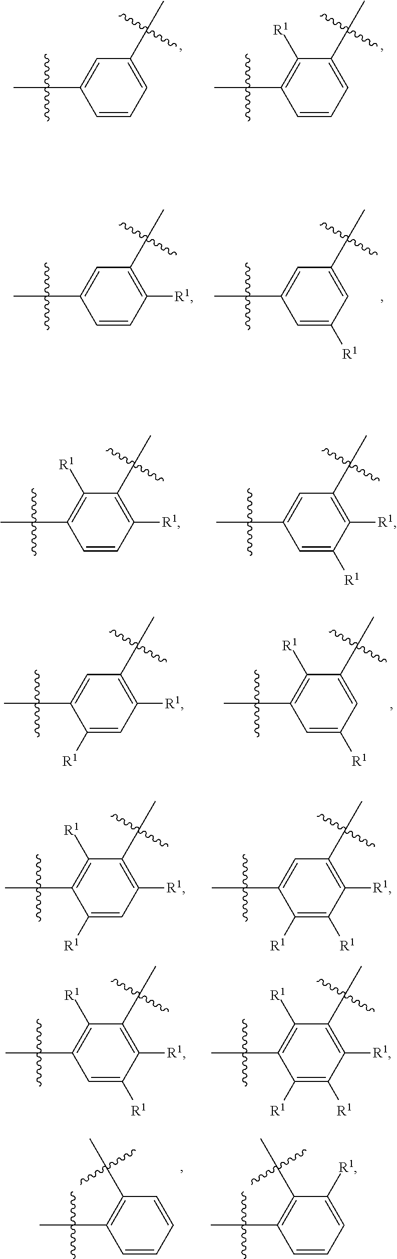

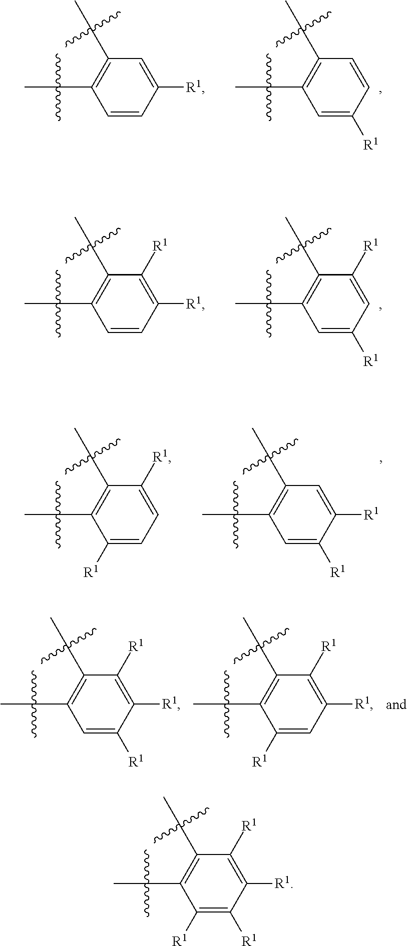

The term “carbocycle” as used herein, unless otherwise specified, refers to a saturated, unsaturated, or aromatic ring in which atom of the ring is carbon. In certain embodiments, the carbocycle group may be saturated, and/or bridged, and/or non-bridged, and/or a fused bicyclic group, and/or a spirocyclic bicyclic group. In certain embodiments, the carbocycle group includes three to ten carbon atoms (i.e., C₃ to C₁₀ carbocycle). In some embodiments, the carbocycle has from three to fifteen carbons (C₃₋₁₅), from three to ten carbons (C₃₋₁₀), from three to seven carbons (C₃₋₇), or from three to six carbons (C₃-C₆). In certain embodiments, the carbocycle group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl.

The term “heterocycle” refers to a saturated, unsaturated or aromatic ring comprising one or more heteroatoms. Exemplary heteroatoms include N, O, Si, P, B, and S atoms where the nitrogen or sulfur atoms may be optionally oxidized, and the nitrogen atoms may be optionally quaternized and the remaining ring atoms of the non-aromatic ring are carbon atoms. Heterocycles include 3- to 10-membered monocyclic rings, 6- to 12-membered bicyclic rings, and 6- to 12-membered bridged rings. In certain embodiments, heterocycle is a monovalent, monocyclic, or multicyclic fully-saturated ring system. In certain embodiments, the heterocycloalkyl or “heterocycle” group may be unsaturated, and/or bridged, and/or non-bridged, and/or a fused bicyclic group, and/or a spirocyclic bicyclic group.

The term “site-specific” refers to a modification of a polypeptide at a predetermined sequence location in the polypeptide. The modification is at a single, predictable residue of the polypeptide with little or no variation. In particular embodiments, a modified amino acid is introduced at that sequence location, for instance recombinantly or synthetically. Similarly, a moiety can be “site-specifically” linked to a residue at a particular sequence location in the polypeptide. In certain embodiments, a polypeptide can comprise more than one site-specific modification.

### 2. Conjugates

Provided herein are conjugates of antibodies to receptor tyrosine kinase orphan receptor 1 (ROR1). The conjugates comprise an antibody to ROR1 covalently linked directly or indirectly, via a linker, to a payload. In some embodiments, the conjugate comprises an antibody that specifically binds to receptor tyrosine kinase orphan receptor 1 (ROR1) linked site-specifically to at least one payload moiety, and the antibody comprises one or more non-natural amino acids. In certain embodiments, the antibody is linked to one payload. In further embodiments, the antibody is linked to more than one payload. In certain embodiments, the antibody is linked to two, three, four, five, six, seven, eight, nine, ten, or more payloads.

The payload can be any payload deemed useful by the practitioner of skill. In certain embodiments, the payload is a therapeutic moiety. In certain embodiments, the payload is a diagnostic moiety, e.g., a label. Useful payloads are described in the sections and examples below.

The linker can be any linker capable of forming at least one bond to the antibody and at least one bond to a payload. Useful linkers are described the sections and examples below.

In the conjugates provided herein, the antibody can be any antibody with binding specificity for ROR1. The ROR1 can be from any species. In certain embodiments, the ROR1 is a vertebrate ROR1. In certain embodiments, the ROR1 is a mammalian ROR1. In certain embodiments, the ROR1 is human ROR1. In certain embodiments, the ROR1 is mouse ROR1. In certain embodiments, the ROR1 is cynomolgus ROR1.

In certain embodiments, the antibody to ROR1 competes with an antibody described herein for binding. In certain embodiments, the antibody to ROR1 binds to the same epitope as an antibody described herein.

The antibody is typically a protein comprising multiple polypeptide chains. In certain embodiments, the antibody is a heterotetramer comprising two identical light (L) chains and two identical heavy (H) chains. Each light chain can be linked to a heavy chain by one covalent disulfide bond. Each heavy chain can be linked to the other heavy chain by one or more covalent disulfide bonds. Each heavy chain and each light chain can also have one or more intrachain disulfide bonds. As is known to those of skill in the art, each heavy chain typically comprises a variable domain (V_{H}) followed by a number of constant domains. Each light chain typically comprises a variable domain at one end (V_{L}) and a constant domain. As is known to those of skill in the art, antibodies typically have selective affinity for their target molecules, i.e., antigens.

The antibodies provided herein can have any antibody form known to those of skill in the art. They can be full-length, or fragments. Exemplary full-length antibodies include IgA, IgA1, IgA2, IgD, IgE, IgG, IgG1, IgG2, IgG3, IgG4, IgM, etc. Exemplary fragments include Fv, Fab, Fc, scFv, scFv-Fc, and etc.

In certain embodiments, the antibody of the conjugate comprises one, two, three, four, five, or six of the CDR sequences described herein. In certain embodiments, the antibody of the conjugate comprises a heavy chain variable domain (V_{H}) described herein. In certain embodiments, the antibody of the conjugate comprises a light chain variable domain (V_{L}) described herein. In certain embodiments, the antibody of the conjugate comprises a heavy chain variable domain (V_{H}) described herein and a light chain variable domain (V_{L}) described herein. In certain embodiments, the antibody of the conjugate comprises a paired heavy chain variable domain and a light chain variable domain described herein (V_{H}-V_{L} pair).

In certain embodiments, the antibody of the conjugate comprises any of the amino acid sequences of the antibodies described above. In certain embodiments, the antibody comprises any of the amino acid sequences above with up to 10 amino acid substitutions. In certain embodiments, the antibody comprises any of the amino acid sequences above with up to 9 amino acid substitutions. In certain embodiments, the antibody comprises any of the amino acid sequences above with up to 8 amino acid substitutions. In certain embodiments, the antibody comprises any of the amino acid sequences above with up to 7 amino acid substitutions. In certain embodiments, the antibody comprises any of the amino acid sequences above with up to 6 amino acid substitutions. In certain embodiments, the antibody comprises any of the amino acid sequences above with up to 5 amino acid substitutions. In certain embodiments, the antibody comprises any of the amino acid sequences above with up to 4 amino acid substitutions. In certain embodiments, the antibody comprises any of the amino acid sequences above with up to 3 amino acid substitutions. In certain embodiments, the antibody comprises any of the amino acid sequences above with up to 2 amino acid substitutions. In certain embodiments, the antibody comprises any of the amino acid sequences above with up to 1 conservative amino acid substitution. In some embodiments, the amino acid substitutions are conservative amino acid substitutions. For example, in certain embodiments, the antibody comprises any of the amino acid sequences above with up to 10 conservative amino acid substitutions. In certain embodiments, the antibody comprises any of the amino acid sequences above with up to 9 conservative amino acid substitutions. In certain embodiments, the antibody comprises any of the amino acid sequences above with up to 8 conservative amino acid substitutions. In certain embodiments, the antibody comprises any of the amino acid sequences above with up to 7 conservative amino acid substitutions. In certain embodiments, the antibody comprises any of the amino acid sequences above with up to 6 conservative amino acid substitutions. In certain embodiments, the antibody comprises any of the amino acid sequences above with up to 5 conservative amino acid substitutions. In certain embodiments, the antibody comprises any of the amino acid sequences above with up to 4 conservative amino acid substitutions. In certain embodiments, the antibody comprises any of the amino acid sequences above with up to 3 conservative amino acid substitutions. In certain embodiments, the antibody comprises any of the amino acid sequences above with up to 2 conservative amino acid substitutions. In certain embodiments, the antibody comprises any of the amino acid sequences above with up to 1 conservative amino acid substitution.

In certain embodiments, the antibody conjugate can be formed from an antibody that comprises one or more reactive groups. In certain embodiments, the antibody conjugate can be formed from an antibody comprising all naturally encoded amino acids. Those of skill in the art will recognize that several naturally encoded amino acids include reactive groups capable of conjugation to a payload or to a linker. These reactive groups include cysteine side chains, lysine side chains, and amino-terminal groups. In these embodiments, the antibody conjugate can comprise a payload or linker linked to the residue of an antibody reactive group. In these embodiments, the payload precursor or linker precursor comprises a reactive group capable of forming a bond with an antibody reactive group. Typical reactive groups include maleimide groups, activated carbonates (including but not limited to, p-nitrophenyl ester), activated esters (including but not limited to, N-hydroxysuccinimide, p-nitrophenyl ester, and aldehydes). Particularly useful reactive groups include maleimide and succinimide, for instance N-hydroxysuccinimide, for forming bonds to cysteine and lysine side chains. Additional reactive groups include alkynes, for example strained alkynes, and azides, for forming bonds to non-natural amino acids incorporated in antibody polypeptide chains. Further reactive groups are described in the sections and examples below.

In certain embodiments, the antibody comprises one or more modified amino acids having a reactive group, as described herein. Typically, the modified amino acid is not a naturally encoded amino acid. These modified amino acids can comprise a reactive group useful for forming a covalent bond to a linker precursor or to a payload precursor. One of skill in the art can use the reactive group to link the polypeptide to any molecular entity capable of forming a covalent bond to the modified amino acid. Thus, provided herein are conjugates comprising an antibody comprising a modified amino acid residue linked to a payload directly or indirectly via a linker. Exemplary modified amino acids are described in the sections below. Generally, the modified amino acids have reactive groups capable of forming bonds to linkers or payloads with complementary reactive groups.

The non-natural amino acids are positioned at select locations in a polypeptide chain of the antibody. These locations were identified as providing optimum sites for substitution with the non-natural amino acids. Each site is capable of bearing a non-natural amino acid with optimum structure, function and/or methods for producing the antibody.

In certain embodiments, a site-specific position for substitution provides an antibody that is stable. Stability can be measured by any technique apparent to those of skill in the art.

In certain embodiments, a site-specific position for substitution provides an antibody that has optimal functional properties. For instance, the antibody can show little or no loss of binding affinity for its target antigen compared to an antibody without the site-specific non-natural amino acid. In certain embodiments, the antibody can show enhanced binding compared to an antibody without the site-specific non-natural amino acid.

In certain embodiments, a site-specific position for substitution provides an antibody that can be made advantageously. For instance, in certain embodiments, the antibody shows advantageous properties in its methods of synthesis, discussed below. In certain embodiments, the antibody can show little or no loss in yield in production compared to an antibody without the site-specific non-natural amino acid. In certain embodiments, the antibody can show enhanced yield in production compared to an antibody without the site-specific non-natural amino acid. In certain embodiments, the antibody can show little or no loss of tRNA suppression compared to an antibody without the site-specific non-natural amino acid. In certain embodiments, the antibody can show enhanced tRNA suppression in production compared to an antibody without the site-specific non-natural amino acid.

In certain embodiments, a site-specific position for substitution provides an antibody that has advantageous solubility. In certain embodiments, the antibody can show little or no loss in solubility compared to an antibody without the site-specific non-natural amino acid. In certain embodiments, the antibody can show enhanced solubility compared to an antibody without the site-specific non-natural amino acid.

In certain embodiments, a site-specific position for substitution provides an antibody that has advantageous expression. In certain embodiments, the antibody can show little or no loss in expression compared to an antibody without the site-specific non-natural amino acid. In certain embodiments, the antibody can show enhanced expression compared to an antibody without the site-specific non-natural amino acid.

In certain embodiments, a site-specific position for substitution provides an antibody that has advantageous folding. In certain embodiments, the antibody can show little or no loss in proper folding compared to an antibody without the site-specific non-natural amino acid. In certain embodiments, the antibody can show enhanced folding compared to an antibody without the site-specific non-natural amino acid.

In certain embodiments, a site-specific position for substitution provides an antibody that is capable of advantageous conjugation. As described below, several non-natural amino acids have side chains or functional groups that facilitate conjugation of the antibody to a second agent, either directly or via a linker. In certain embodiments, the antibody can show enhanced conjugation efficiency compared to an antibody without the same or other non-natural amino acids at other positions. In certain embodiments, the antibody can show enhanced conjugation yield compared to an antibody without the same or other non-natural amino acids at other positions. In certain embodiments, the antibody can show enhanced conjugation specificity compared to an antibody without the same or other non-natural amino acids at other positions.

The one or more non-natural amino acids are located at selected site-specific positions in at least one polypeptide chain of the antibody. The polypeptide chain can be any polypeptide chain of the antibody without limitation, including either light chain or either heavy chain. The site-specific position can be in any domain of the antibody, including any variable domain and any constant domain.

In certain embodiments, the antibodies provided herein comprise one non-natural amino acid at a site-specific position. In certain embodiments, the antibodies provided herein comprise two non-natural amino acids at site-specific positions. In certain embodiments, the antibodies provided herein comprise three non-natural amino acids at site-specific positions. In certain embodiments, the antibodies provided herein comprise more than three non-natural amino acids at site-specific positions. In certain embodiments, the antibodies provided herein comprise four non-natural amino acids at site-specific positions.

In certain embodiments, the antibodies provided herein comprise one or more non-natural amino acids each at a position selected from the group consisting of heavy chain or light chain residues HC-F404, HC-K121, HC-Y180, HC-F241, HC-221, LC-T22, LC-S7, LC-N152, LC-K42, LC-E161, LC-D170, HC-S136, HC-S25, HC-A40, HC-S119, HC-S190, HC-K222, HC-R19, HC-Y52, or HC-S70 according to the Kabat or Chothia or EU numbering scheme, or a post-translationally modified variant thereof. In certain embodiments, the antibodies provided herein comprise one or more non-natural amino acids each at a position selected from the group consisting of heavy chain or light chain residues HC-F404, HC-Y180, HC-F241, LC-K42, and LC-E161, according to the Kabat or Chothia or EU numbering scheme, or a post-translationally modified variant thereof. In certain embodiments, the antibodies provided herein comprise a non-natural amino acid at position HC-F404 according to the Kabat or Chothia or EU numbering scheme, or a post-translationally modified variant thereof. In certain embodiments, the antibodies provided herein comprise a non-natural amino acid at position HC-Y180, according to the Kabat or Chothia or EU numbering scheme, or a post-translationally modified variant thereof. In certain embodiments, the antibodies provided herein comprise non-natural amino acids at positions HC-F404 and HC-Y180, according to the Kabat or Chothia or EU numbering scheme, or a post-translationally modified variant thereof. In certain embodiments, the antibodies provided herein comprise a non-natural amino acid at position HC-F241 according to the Kabat or Chothia or EU numbering scheme, or a post-translationally modified variant thereof. In certain embodiments, the antibodies provided herein comprise a non-natural amino acid at position LC-K42 according to the Kabat or Chothia or EU numbering scheme, or a post-translationally modified variant thereof. In certain embodiments, the antibodies provided herein comprise a non-natural amino acid at position LC-E161 according to the Kabat or Chothia or EU numbering scheme, or a post-translationally modified variant thereof. In certain embodiments, the antibodies provided herein comprise non-natural amino acids at positions HC-F404, HC-Y180, and LC-K42, according to the Kabat or Chothia or EU numbering scheme, or a post-translationally modified variant thereof. In certain embodiments, the antibodies provided herein comprise non-natural amino acids at positions HC-F404, HC-Y180, LC-K42, and LC-E161, according to the Kabat or Chothia or EU numbering scheme, or a post-translationally modified variant thereof. In certain embodiments, the antibodies provided herein comprise non-natural amino acids at positions HC-F404, HC-Y180, and HC-F241, according to the Kabat or Chothia or EU numbering scheme, or a post-translationally modified variant thereof. In certain embodiments, the antibodies provided herein comprise non-natural amino acids at positions HC-F404, HC-Y180, HC-F241, and LC-K42, according to the Kabat or Chothia or EU numbering scheme, or a post-translationally modified variant thereof. In certain embodiments, the antibodies provided herein comprise non-natural amino acids at positions HC-F404, HC-Y180, HC-F241, and LC-K42, according to the Kabat or Chothia or EU numbering scheme, or a post-translationally modified variant thereof. In these designations, HC indicates a heavy chain residue, and LC indicates a light chain residue.

In some aspects, the present disclosure provides conjugates according to the following formula:

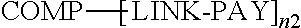

or a pharmaceutically acceptable salt, solvate, stereoisomer, regioisomer, or tautomer thereof, wherein:
COMP is a residue of an anti-ROR1 antibody;
PAY is a payload moiety;
LINK is a linker; and
n2 is an integer from 1 to 10,
   wherein COMP comprises one or more non-natural amino acids.

In certain embodiments, provided herein are conjugates according to Formula (C1) or (C2):

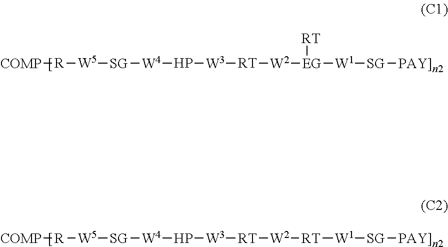

or a pharmaceutically acceptable salt, solvate, stereoisomer, regioisomer, or tautomer thereof, wherein:
COMP is a residue of an anti-ROR1 antibody;
PAY is a payload moiety;
W¹, W², W³, W⁴, and W⁵ are each independently a single bond, absent, or a divalent attaching group;
EG is absent, or an eliminator group;
each RT is a release trigger group, in the backbone of Formula (C1) or (C2) or bonded to EG, wherein each RT is optional;
HP is a single bond, absent, or a divalent hydrophilic group;
each SG is a single bond, absent, or a divalent spacer group;
R is hydrogen, a terminal conjugating group, or a divalent residue of a terminal conjugating group; and
n2 is an integer from 1 to 10.

In some embodiments, a conjugate according to Formula (C1) or (C2) comprises n2 number of linked PAY moieties, wherein n2 is an integer from 1 to 10. In some embodiments, n2 is 2. In some embodiments, n2 is 3. In some embodiments, n2 is 4. In some embodiments, n2 is 5. In some embodiments, n2 is 6. In some embodiments, n2 is 7. In some embodiments, n2 is 8. In some embodiments, n2 is 10.

### Attaching Groups

Attaching groups facilitate incorporation of eliminator groups, release trigger groups, hydrophobic groups, spacer groups, and/or conjugating groups into a compound. Useful attaching groups are known to, and are apparent to, those of skill in the art. Examples of useful attaching groups are provided herein. In certain embodiments, attaching groups are designated W¹, W², W³, W⁴, or W⁵. In certain embodiments, an attaching group can comprise a divalent ketone, divalent ester, divalent ether, divalent amide, divalent amine, alkylene, arylene, sulfide, disulfide, carbonylene, or a combination thereof. In certain embodiments an attaching group can comprise —C(O)—, —O—, —C(O)NH—, —C(O)NH-alkyl-, —OC(O)NH—, —SC(O)NH—, —NH—, —NH-alkyl-, —N(CH3)CH2CH2N(CH3)-, —S—, —S—S—, —OCH2CH2O—, or the reverse (e.g. —NHC(O)—) thereof, or a combination thereof.

### Eliminator Groups

Eliminator groups facilitate separation of a biologically active portion of a compound or conjugate described herein from the remainder of the compound or conjugate in vivo and/or in vitro. Eliminator groups can also facilitate separation of a biologically active portion of a compound or conjugate described herein in conjunction with a release trigger group. For example, the eliminator group and the release trigger group can react in a Releasing Reaction to release a biologically active portion of a compound or conjugate described herein from the compound or conjugate in vivo and/or in vitro. Upon initiation of the Releasing Reaction by the release trigger, the eliminator group cleaves the biologically active moiety, or a prodrug form of the biologically active moiety, and forms a stable, non-toxic entity that has no further effect on the activity of the biologically active moiety.

In certain embodiments, the eliminator group is designated EG herein. Useful eliminator groups include those described herein. In certain embodiments, the eliminator group is:

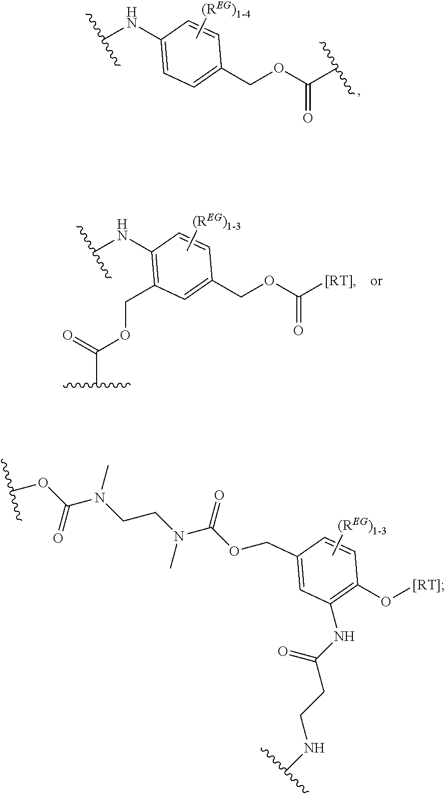

wherein R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the second and third structures, those of skill will recognize that EG is bonded to an RT that is not within the backbone of formula (C1) as indicated in the above description of formula (C1). In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro. In certain embodiments, the eliminator group is

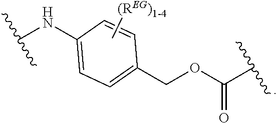

In certain embodiments, the eliminator group is

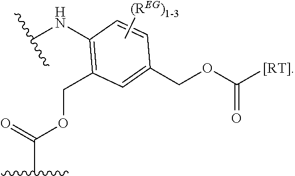

In certain embodiments, the eliminator group is

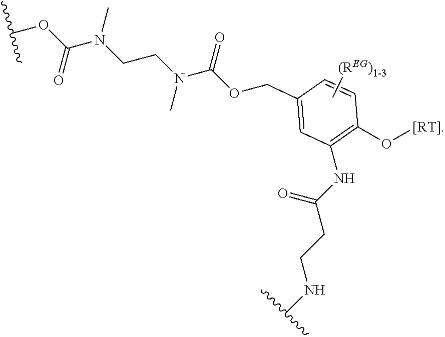

In some embodiments, provided herein is a conjugate according to Formula (C1) or (C2) or a pharmaceutically acceptable salt, solvate, stereoisomer, or tautomer thereof; wherein EG comprises phenylene, carboxylene, amine, or a combination thereof. In some embodiments, the eliminator group is:

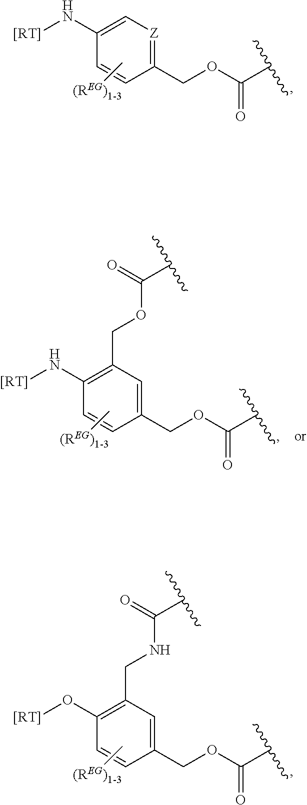

wherein Z may be CH or N, R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the first and second structures, those of skill will recognize that EG is bonded to an RT that is not within the backbone of formula (C1) as indicated in the above description of formula (C1). In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro. In some embodiments, each R^{EG} in the EG is hydrogen. In certain embodiments, the eliminator group is

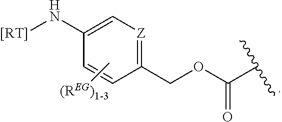

In certain embodiments, the eliminator group is

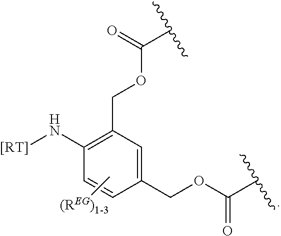

In certain embodiments, the eliminator group is

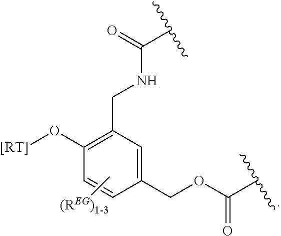

### Release Trigger Groups

Release trigger groups facilitate separation of a biologically active portion of a compound or conjugate described herein from the remainder of the compound or conjugate in vivo and/or in vitro. Release trigger groups can also facilitate separation of a biologically active portion of a compound or conjugate described herein in conjunction with an eliminator group. For example, the eliminator group and the release trigger group can react in a Releasing Reaction to release a biologically active portion of a compound or conjugate described herein from the compound or conjugate in vivo and/or in vitro. In certain embodiment, the release trigger can act through a biologically-driven reaction with high tumor: nontumor specificity, such as the proteolytic action of an enzyme overexpressed in a tumor environment.

In certain embodiments, the release trigger group is designated RT herein. In certain embodiments, RT is divalent and bonded within the backbone of formula (C1). In other embodiments, RT is monovalent and bonded to EG as depicted above. Useful release trigger groups include those described herein. In certain embodiments, the release trigger group comprises a residue of a natural or non-natural amino acid or residue of a sugar ring. In certain embodiments, the release trigger group is:

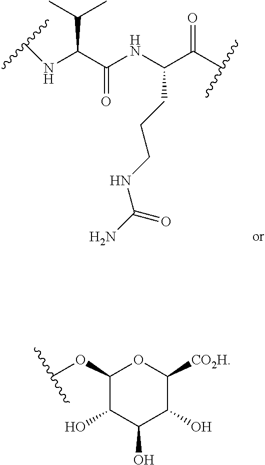 [SMILES: *N[C@H](C(=O)N[C@@H](CCCNC(N)=O)C(*)=O)C(C)C ; *O[C@H]1O[C@@H](C(=O)O)[C@H](O)[C@@H](O)[C@@H]1O]

Those of skill will recognize that the first structure is divalent and can be bonded within the backbone of Formula (C1) or as depicted in Formula (C2), and that the second structure is monovalent and can be bonded to EG as depicted in formula (C1) above. In certain embodiments, the release trigger group is

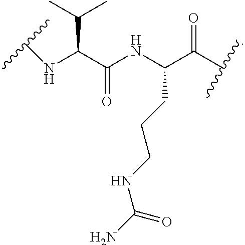 [SMILES: *N[C@H](C(=O)N[C@@H](CCCNC(N)=O)C(*)=O)C(C)C]

In certain embodiments, the release trigger group is

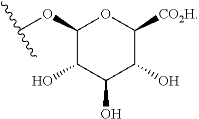 [SMILES: *O[C@H]1O[C@@H](C(=O)O)[C@H](O)[C@@H](O)[C@@H]1O]

In some embodiments, the release trigger group is a protease-cleavable R₁-Val-X peptide having the structure of:

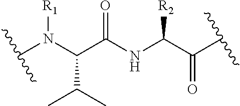

wherein R₁ is H or

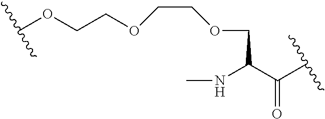

and R₂ is CH₃, CH₂CH₂CO₂H, or (CH₂)₃NHCONH₂; a legumain-cleavable Ala-Ala-Asn or Ala-Ala-Asp peptide having the structure of:

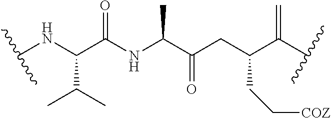

where Z is OH or NH₂; or [symbol] -glucuronidase-cleavable [symbol] -glucuronide having the structure of:

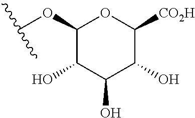 [SMILES: *O[C@H]1O[C@@H](C(=O)O)[C@H](O)[C@@H](O)[C@@H]1O]

or a Val-Lys-Gly peptide having the structure of:

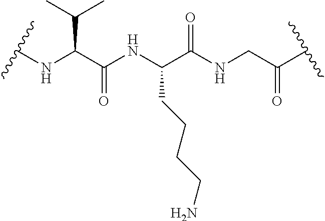 [SMILES: *N[C@H](C(=O)N[C@@H](CCCCN)C(=O)NCC(*)=O)C(C)C]

Those of skill will recognize that

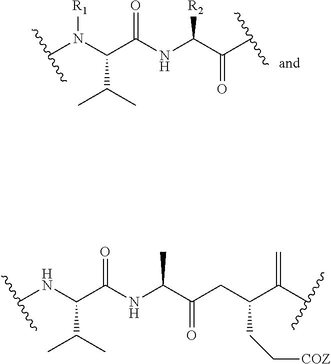

are divalent structures and can be bonded within the backbone of Formula (C1) or as depicted in Formula (C2). The structure

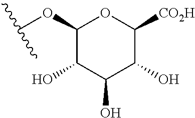 [SMILES: *O[C@H]1O[C@@H](C(=O)O)[C@H](O)[C@@H](O)[C@@H]1O]

is monovalent and can be bonded to EG as depicted in formula (C1) above.

In certain embodiments, the release trigger is selected from the group consisting of Val-Lys-Gly, Val-Ala-Asp, Ala-Ala-Ala, Val-Lys, Gly-Gly-Gly, Val-Ala, Gly-Gly-Phe-Gly, Val-Cit, Val-Cit, Val-Glu, Ala-Ala-Asn, and Gly. In certain embodiments, the release trigger further comprises a non-natural amino acid. In certain embodiments, the non-natural amino acid is according to

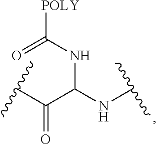

where POLY is a polymer, for instance a hydrophilic polymer. In certain embodiments, the non-natural amino acid is according to

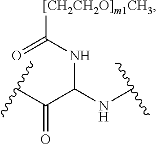

where m1 is an integer from 1 to 25, for instance 8, 9, 10, 11, 12, 13, 14, 15, 20, or 25.

### Hydrophilic Groups

Hydrophilic groups facilitate increasing the hydrophilicity of the compounds described herein. It is believed that increased hydrophilicity allows for greater solubility in aqueous solutions, such as aqueous solutions found in biological systems. Hydrophilic groups can also function as spacer groups, which are described in further detail herein.

In certain embodiments, the hydrophilic group is designated HP herein. Useful hydrophilic groups include those described herein. In certain embodiments, the hydrophilic group is a divalent poly(ethylene glycol). In certain embodiments, the hydrophilic group is a divalent poly(ethylene glycol) according to the formula:

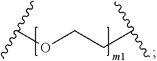

wherein m1 is an integer from 1 to 13, optionally 1 to 4, optionally 2 to 4, or optionally 4 to 8. In certain embodiments, m1 is 4. In certain embodiments, m1 is 12. In certain embodiments, m1 is 13.

In some embodiments, the hydrophilic group is a divalent poly(ethylene glycol) having the following formula:

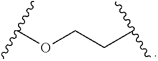 [SMILES: *CCO*]

In some other embodiments, the hydrophilic group is a divalent poly(ethylene glycol) having the following formula:

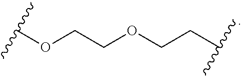 [SMILES: *CCOCCO*]

In other embodiments, the hydrophilic group is a divalent poly(ethylene glycol) having the following formula:

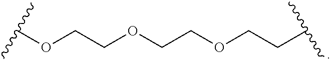 [SMILES: *CCOCCOCCO*]

In other embodiments, the hydrophilic group is a divalent poly(ethylene glycol) having the following formula:

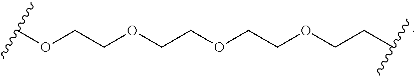 [SMILES: *CCOCCOCCOCCO*]

In some embodiments, the hydrophilic group is a sulfonic acid. In some embodiments, the hydrophilic group is the side chain of cysteic acid. In some embodiments, the hydrophilic group can bear a chain-presented sulfonic acid having the formula:

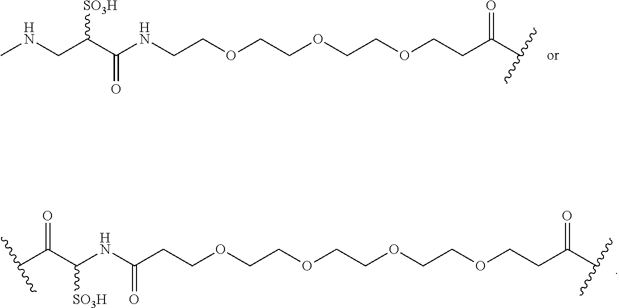 [SMILES: *C(=O)CCOCCOCCOCCNC(=O)C(CNC)S(=O)(=O)O ; *C(=O)CCOCCOCCOCCOCCC(=O)NC(C(*)=O)S(=O)(=O)O]

In certain embodiments, the hydrophilic group is the side chain of a non-natural amino acid according to

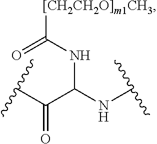

where m1 is an integer from 1 to 25, for instance 8, 9, 10, 11, 12, 13, 14, 15, 20, or 25.

### Spacer Groups

Spacer groups facilitate spacing of the conjugating group from the other groups of the compounds described herein. This spacing can lead to more efficient conjugation of the compounds described herein to an antibody as well as more efficient cleavage of the active catabolite. The spacer group can also stabilize the conjugating group and lead to improved overall antibody-drug conjugate properties.

In certain embodiments, the spacer group is designated SP herein. Useful spacer groups include those described herein. In certain embodiments, the spacer group is:

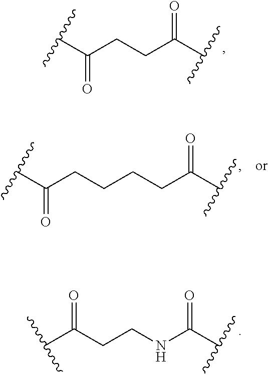 [SMILES: *C(=O)CCC(*)=O ; *C(=O)CCCCC(*)=O ; *C(=O)CCNC(*)=O]

In certain embodiments, the spacer group, W⁴, and the hydrophilic group combine to form a divalent poly(ethylene glycol) according to the formula:

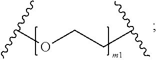

wherein m1 is an integer from 1 to 13, optionally 1 to 4, optionally 2 to 4, or optionally 4 to 8.

In some embodiments, the SP is

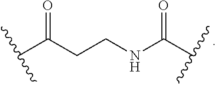 [SMILES: *C(=O)CCNC(*)=O]

In some embodiments, the divalent poly(ethylene glycol) has the following formula:

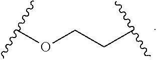 [SMILES: *CCO*]

In some other embodiments, the divalent poly(ethylene glycol) has the following formula:

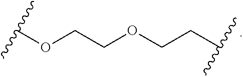 [SMILES: *CCOCCO*]

In other embodiments, the divalent poly(ethylene glycol) has the following formula:

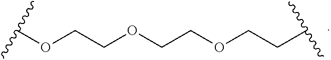 [SMILES: *CCOCCOCCO*]

In other embodiments, the divalent poly(ethylene glycol) has the following formula:

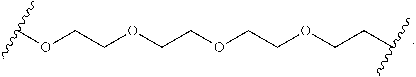 [SMILES: *CCOCCOCCOCCO*]

In some embodiments, the spacer group can bear a chain-presented sulfonic acid having the formula:

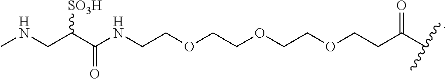 [SMILES: *C(=O)CCOCCOCCOCCNC(=O)C(CNC)S(=O)(=O)O]

In some embodiments, the spacer group is a diamine. In some embodiments, the spacer group is according to

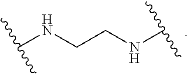 [SMILES: *NCCN*]

In some embodiments, the spacer group comprises fused rings or spiro rings. In some embodiments, the spacer group is according to

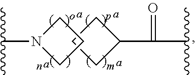

where each n^{a}, m^{a}, o^{a}, and p^{a} is an integer independently selected from 1, 2, 3, 4, and 5. In some embodiments, the spacer group is according to

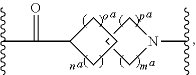

where each n^{a}, m^{a}, o^{a}, and p^{a} is an integer independently selected from 1, 2, 3, 4, and 5. In some embodiments, the spacer group is selected from the group consisting of:

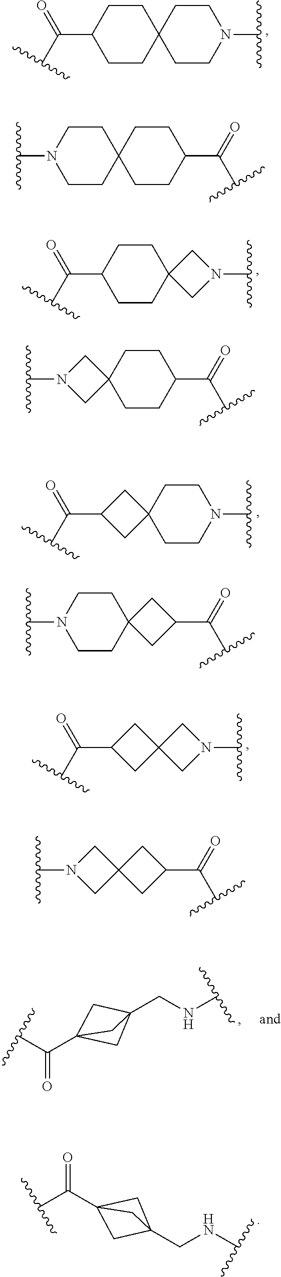

### Conjugating Groups and Residues Thereof

Conjugating groups facilitate conjugation of the payloads described herein to a second compound, such as an antibody described herein. In certain embodiments, the conjugating group is designated R herein. Conjugating groups can react via any suitable reaction mechanism known to those of skill in the art. In certain embodiments, a conjugating group reacts through a [3+2]alkyne-azide cycloaddition reaction, inverse-electron demand Diels-Alder ligation reaction, thiol-electrophile reaction, or carbonyl-oxyamine reaction, as described in detail herein. In certain embodiments, the conjugating group comprises an alkyne, strained alkyne, tetrazine, thiol, para-acetyl-phenylalanine residue, oxyamine, maleimide, or azide. In certain embodiments, the conjugating group is:

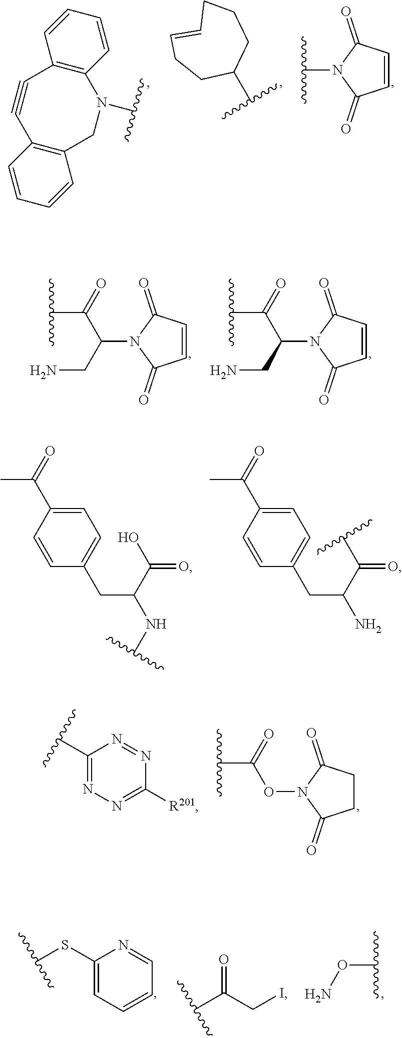

—N₃, or —SH; wherein R²⁰¹ is lower alkyl. In an embodiment, R²⁰¹ is methyl, ethyl, or propyl. In an embodiment, R²⁰¹ is methyl. Additional conjugating groups are described in, for example, U.S. Patent Publication No. 2014/0356385, U.S. Patent Publication No. 2013/0189287, U.S. Patent Publication No. 2013/0251783, U.S. Pat. Nos. 8,703,936, 9,145,361, 9,222,940, and 8,431,558.

After conjugation, a divalent residue of the conjugating group is formed and is bonded to the residue of an antibody. The structure of the divalent residue is determined by the type of conjugation reaction employed to form the conjugate.

In certain embodiments when a conjugate is formed through a [3+2]alkyne-azide cycloaddition reaction, the divalent residue of the conjugating group comprises a triazole ring or fused cyclic group comprising a triazole ring. In certain embodiment when a conjugate is formed through a strain-promoted [3+2]alkyne-azide cycloaddition (SPAAC) reaction, the divalent residue of the conjugating group is:

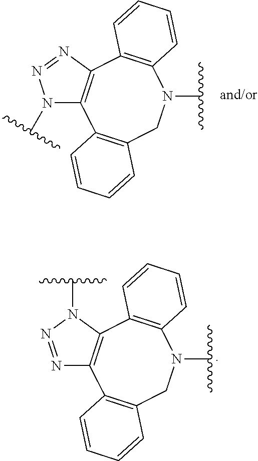 [SMILES: *N1Cc2ccccc2-c2c(nnn2*)-c2ccccc21 ; *N1Cc2ccccc2-c2nnn(*)c2-c2ccccc21]

In certain embodiments when a conjugate is formed through a tetrazine inverse electron demand Diels-Alder ligation reaction, the divalent residue of the conjugating group comprises a fused bicyclic ring having at least two adjacent nitrogen atoms in the ring. In certain embodiments when a conjugate is formed through a tetrazine inverse electron demand Diels-Alder ligation reaction, the divalent residue of the conjugating group is:

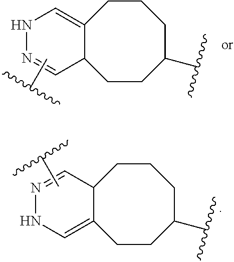

In certain embodiments when a conjugate is formed through a thiol-maleimide reaction, the divalent residue of the conjugating group comprises succinimidylene and a sulfur linkage. In certain embodiments when a conjugate is formed through a thiol-maleimide reaction, the divalent residue of the conjugating group is:

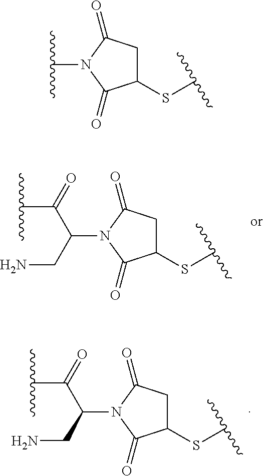 [SMILES: *SC1CC(=O)N(*)C1=O ; *SC1CC(=O)N(C(CN)C(*)=O)C1=O ; *SC1CC(=O)N([C@@H](CN)C(*)=O)C1=O]

In certain embodiments, a conjugate is formed through a thiol-N-hydroxysuccinimide reaction using the following group:

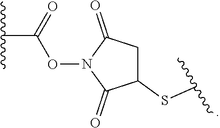 [SMILES: *SC1CC(=O)N(OC(*)=O)C1=O]

The reaction involved for formation of the conjugate comprises the following step:

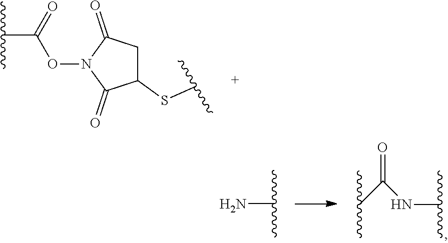

and the resulting divalent residue of the conjugating group is:

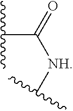 [SMILES: *NC(*)=O]

In certain embodiments when a conjugate is formed through a carbonyl-oxyamine reaction, the divalent residue of the conjugating group comprises a divalent residue of a non-natural amino acid. In certain embodiments when a conjugate is formed through a carbonyl-oxyamine reaction, the divalent residue of the conjugating group is:

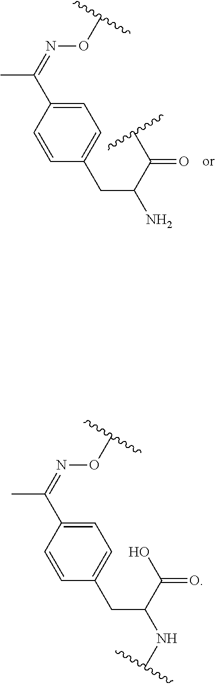 [SMILES: *O/N=C(/C)c1ccc(CC(N)C(*)=O)cc1 ; *NC(Cc1ccc(/C(C)=N\O*)cc1)C(=O)O]

In certain embodiments when a conjugate is formed through a carbonyl-oxyamine reaction, the divalent residue of the conjugating group comprises an oxime linkage. In certain embodiments when a conjugate is formed through a carbonyl-oxyamine reaction, the divalent residue of the conjugating group is:

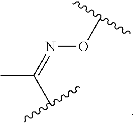 [SMILES: *O/N=C(\*)C]

In an embodiment, provided herein is a conjugate according to Formula (C1) or (C2), or a pharmaceutically acceptable salt, solvate, stereoisomer, or tautomer thereof; wherein R comprises a triazole ring. In an embodiment, provided herein is a conjugate according to Formula (C1) or (C2), or a pharmaceutically acceptable salt, solvate, stereoisomer, or tautomer thereof; wherein R is a triazole ring or fused cyclic group comprising a triazole ring. In an embodiment, provided herein is a conjugate according to Formula (C1) or (C2), or a pharmaceutically acceptable salt, solvate, stereoisomer, or tautomer thereof; wherein R is:

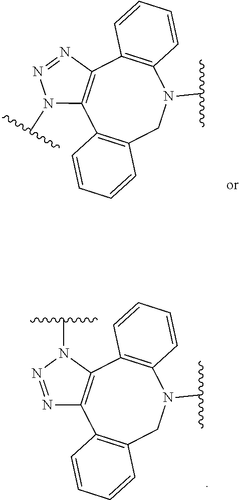 [SMILES: *N1Cc2ccccc2-c2c(nnn2*)-c2ccccc21 ; *N1Cc2ccccc2-c2nnn(*)c2-c2ccccc21]

In an embodiment, provided herein is a conjugate according to Formula (C1) or (C2), or a pharmaceutically acceptable salt, solvate, stereoisomer, or tautomer thereof; wherein R comprises a fused bicyclic ring having at least two adjacent nitrogen atoms in the ring. In an embodiment, provided herein is a conjugate according to Formula (C1) or (C2), or a pharmaceutically acceptable salt, solvate, stereoisomer, or tautomer thereof; wherein R is:

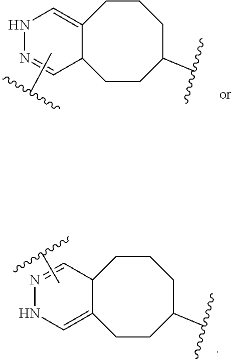

In an embodiment, provided herein is a conjugate according to Formula (C1) or (C2), or a pharmaceutically acceptable salt, solvate, stereoisomer, or tautomer thereof; wherein R comprises a sulfur linkage. In an embodiment, provided herein is a conjugate according to Formula (C1) or (C2), or a pharmaceutically acceptable salt, solvate, stereoisomer, or tautomer thereof; wherein R is:

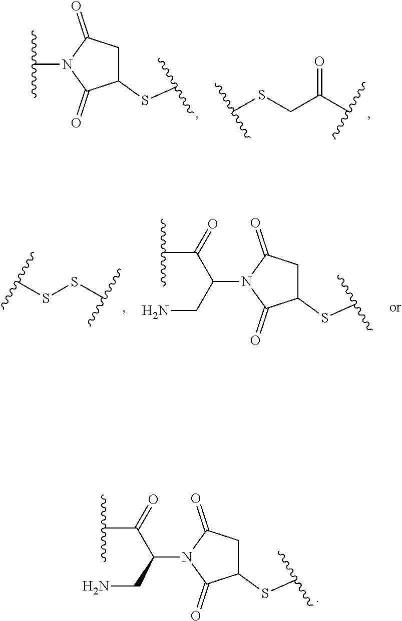

In an embodiment, provided herein is a conjugate according to Formula (C1) or (C2), or a pharmaceutically acceptable salt, solvate, stereoisomer, or tautomer thereof; wherein R comprises a divalent residue of a non-natural amino acid. In an embodiment, provided herein is a conjugate according to Formula (C1) or (C2), or a pharmaceutically acceptable salt, solvate, stereoisomer, or tautomer thereof; wherein R is:

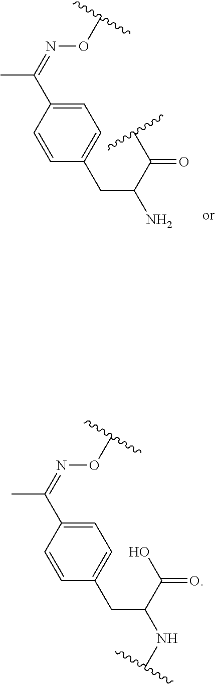 [SMILES: *O/N=C(/C)c1ccc(CC(N)C(*)=O)cc1 ; *NC(Cc1ccc(/C(C)=N\O*)cc1)C(=O)O]

In an embodiment, provided herein is a conjugate according to Formula (C1) or (C2), or a pharmaceutically acceptable salt, solvate, stereoisomer, or tautomer thereof; wherein comprises an amide linkage. In an embodiment, provided herein is a conjugate according to Formula (C1) or (C2), or a pharmaceutically acceptable salt, solvate, stereoisomer, or tautomer thereof; wherein R is:

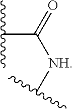 [SMILES: *NC(*)=O]

In an embodiment, provided herein is a conjugate according to Formula (C1) or (C2), or a pharmaceutically acceptable salt, solvate, stereoisomer, or tautomer thereof; wherein comprises an oxime linkage. In an embodiment, provided herein is a conjugate according to Formula (C1) or (C2), or a pharmaceutically acceptable salt, solvate, stereoisomer, or tautomer thereof; wherein R is:

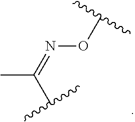 [SMILES: *O/N=C(\*)C]

In an embodiment, provided herein is a conjugate according to Formula (C1) or (C2), or a pharmaceutically acceptable salt, solvate, stereoisomer, or tautomer thereof; wherein R is:

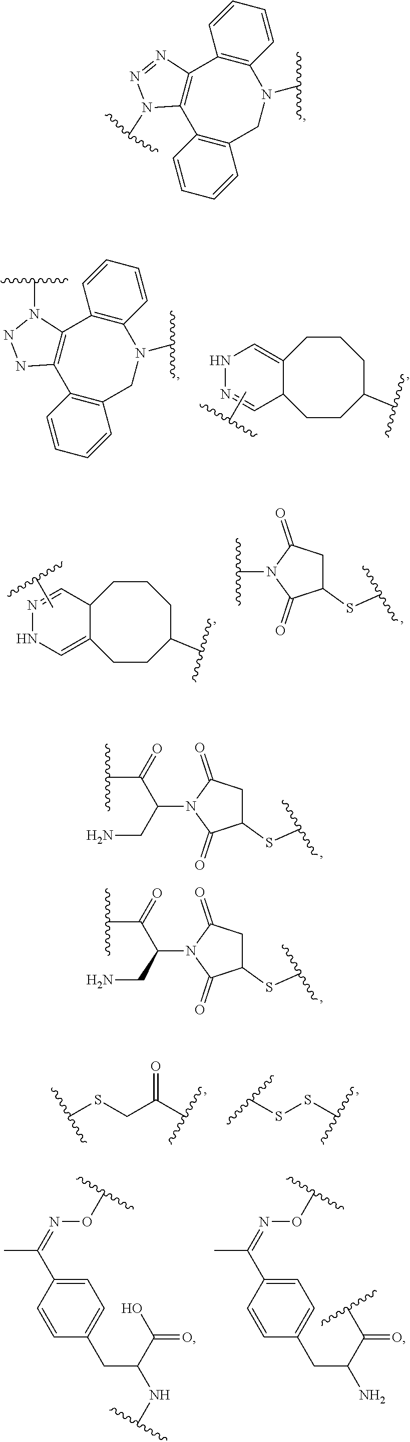

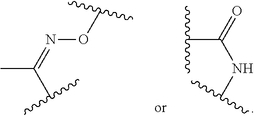

In an embodiment, provided herein is a compound according to Formula (C1) or (C2), or a pharmaceutically acceptable salt, solvate, stereoisomer, or tautomer thereof; wherein COMP is a residue of any compound known to be useful for conjugation to a payload, described herein, and an optional linker, described herein. In an embodiment, provided herein is a compound according to Formula (C1) or (C2), or a pharmaceutically acceptable salt, solvate, stereoisomer, or tautomer thereof; wherein COMP is a residue of an antibody chain.

In an aspect, provided herein is an antibody conjugate comprising payload, described herein, and an optional linker, described herein, linked to an anti-ROR1 antibody, wherein COMP is a residue of the antibody. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a triazole ring or fused cyclic group comprising a triazole ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

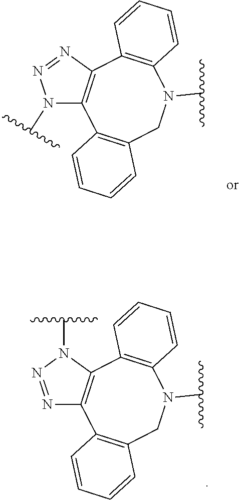 [SMILES: *N1Cc2ccccc2-c2c(nnn2*)-c2ccccc21 ; *N1Cc2ccccc2-c2nnn(*)c2-c2ccccc21]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a fused bicyclic ring, wherein the fused bicyclic ring has at least two adjacent nitrogen atoms in the ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

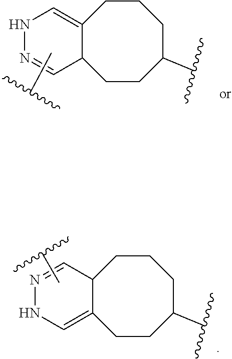

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises a sulfur linkage. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

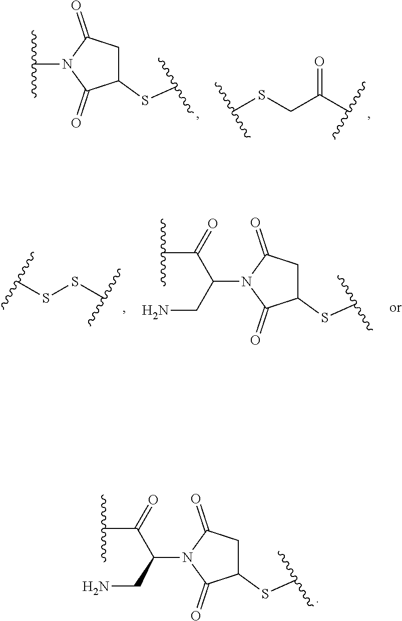

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises a divalent residue of a non-natural amino acid. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

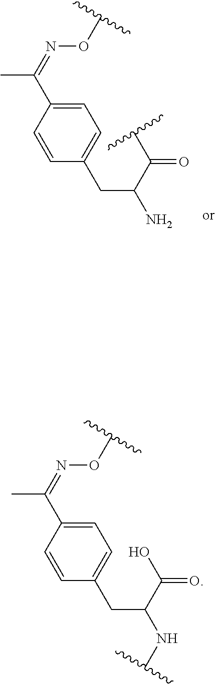 [SMILES: *O/N=C(/C)c1ccc(CC(N)C(*)=O)cc1 ; *NC(Cc1ccc(/C(C)=N\O*)cc1)C(=O)O]

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

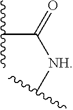 [SMILES: *NC(*)=O]

In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the polypeptide; and R is:

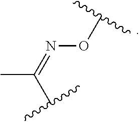 [SMILES: *O/N=C(\*)C]

In an aspect, provided herein is an antibody conjugate comprising a payload, described herein, and an optional linker, described herein, linked to an antibody according to Formula (C1) or (C2), or a pharmaceutically acceptable salt, solvate, stereoisomer, or tautomer thereof, wherein COMP is a residue of the antibody. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a triazole ring or fused cyclic group comprising a triazole ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

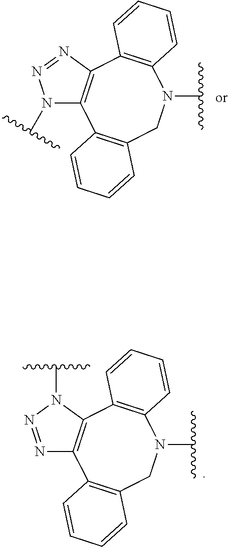 [SMILES: *N1Cc2ccccc2-c2c(nnn2*)-c2ccccc21 ; *N1Cc2ccccc2-c2nnn(*)c2-c2ccccc21]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a fused bicyclic ring, wherein the fused bicyclic ring has at least two adjacent nitrogen atoms in the ring. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

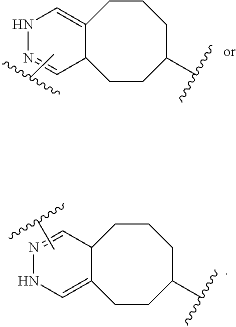

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a sulfur linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

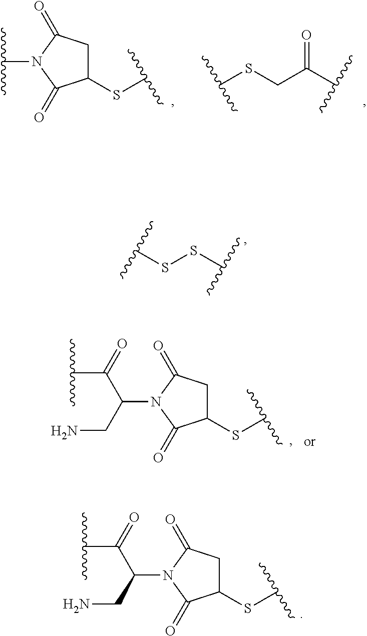

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises a divalent residue of a non-natural amino acid. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

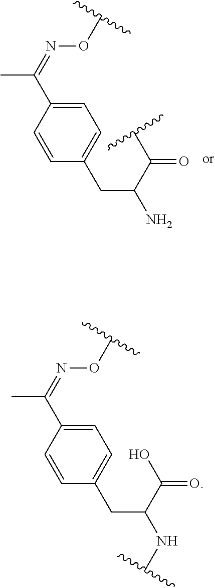 [SMILES: *O/N=C(/C)c1ccc(CC(N)C(*)=O)cc1 ; *NC(Cc1ccc(/C(C)=N\O*)cc1)C(=O)O]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

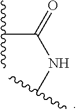 [SMILES: *NC(*)=O]

In an embodiment, provided herein is an antibody conjugate according to Formula (C1) or (C2), or a pharmaceutically acceptable salt, solvate, stereoisomer, or tautomer thereof, wherein: COMP is a residue of the antibody; and R comprises an oxime linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody; and R is:

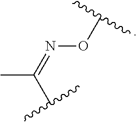 [SMILES: *O/N=C(\*)C]

In an aspect, provided herein is an antibody conjugate comprising a payload, described herein, and an optional linker, described herein, linked to an antibody chain according to Formula (C1) or (C2), or a pharmaceutically acceptable salt, solvate, stereoisomer, or tautomer thereof, wherein COMP is a residue of the antibody chain. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a triazole ring or fused cyclic group comprising a triazole ring. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

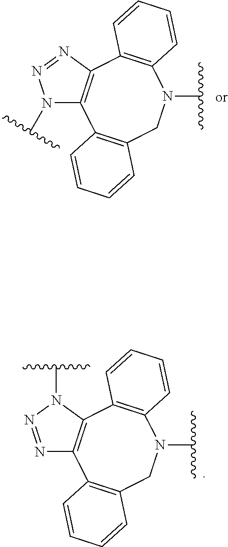 [SMILES: *N1Cc2ccccc2-c2c(nnn2*)-c2ccccc21 ; *N1Cc2ccccc2-c2nnn(*)c2-c2ccccc21]

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a fused bicyclic ring, wherein the fused bicyclic ring has at least two adjacent nitrogen atoms in the ring. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

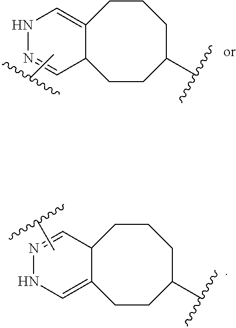

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a sulfur linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

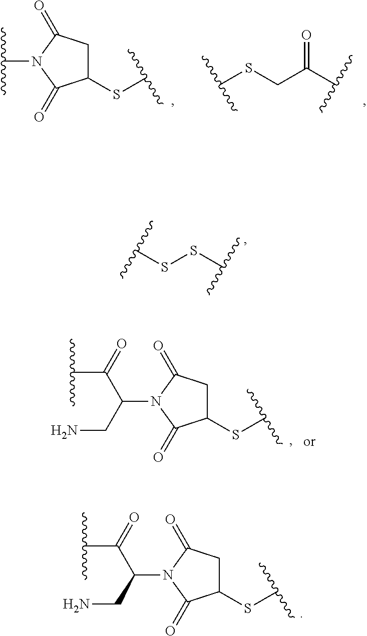

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises a divalent residue of a non-natural amino acid. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

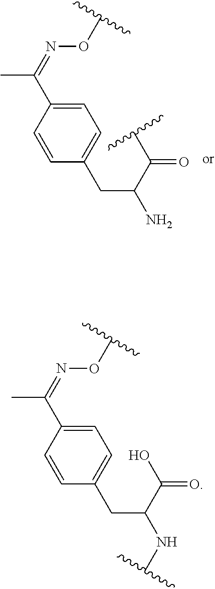 [SMILES: *O/N=C(/C)c1ccc(CC(N)C(*)=O)cc1 ; *NC(Cc1ccc(/C(C)=N\O*)cc1)C(=O)O]

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

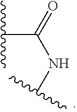 [SMILES: *NC(*)=O]

In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R comprises an amide linkage. In an embodiment, provided herein is an antibody conjugate according to Formula (C1) or (C2), or a pharmaceutically acceptable salt, solvate, stereoisomer, or tautomer thereof, wherein: COMP is a residue of the antibody chain; and R is:

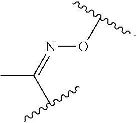 [SMILES: *O/N=C(\*)C]

### Conjugates

In an embodiment, provided herein is a conjugate according to any of the following formulas, where COMP indicates a residue of the anti-ROR1 antibody and PAY indicates a payload moiety:

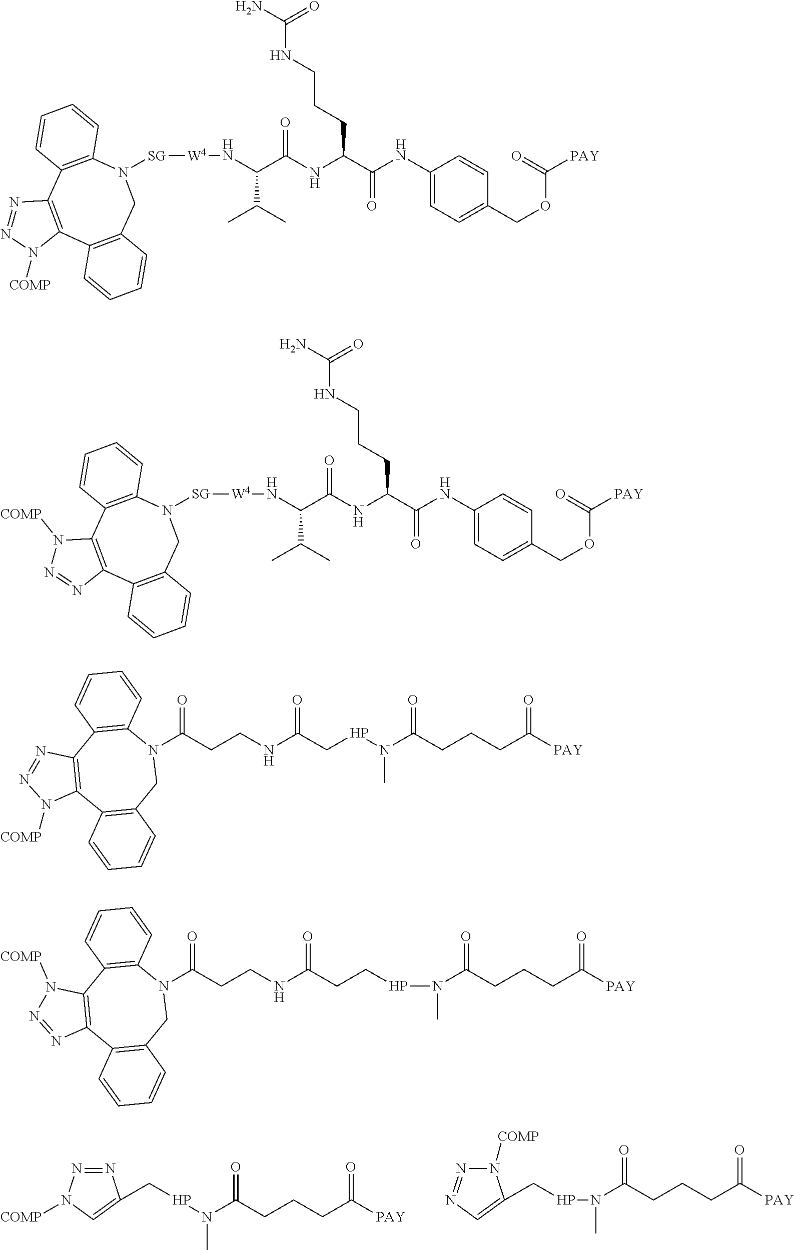

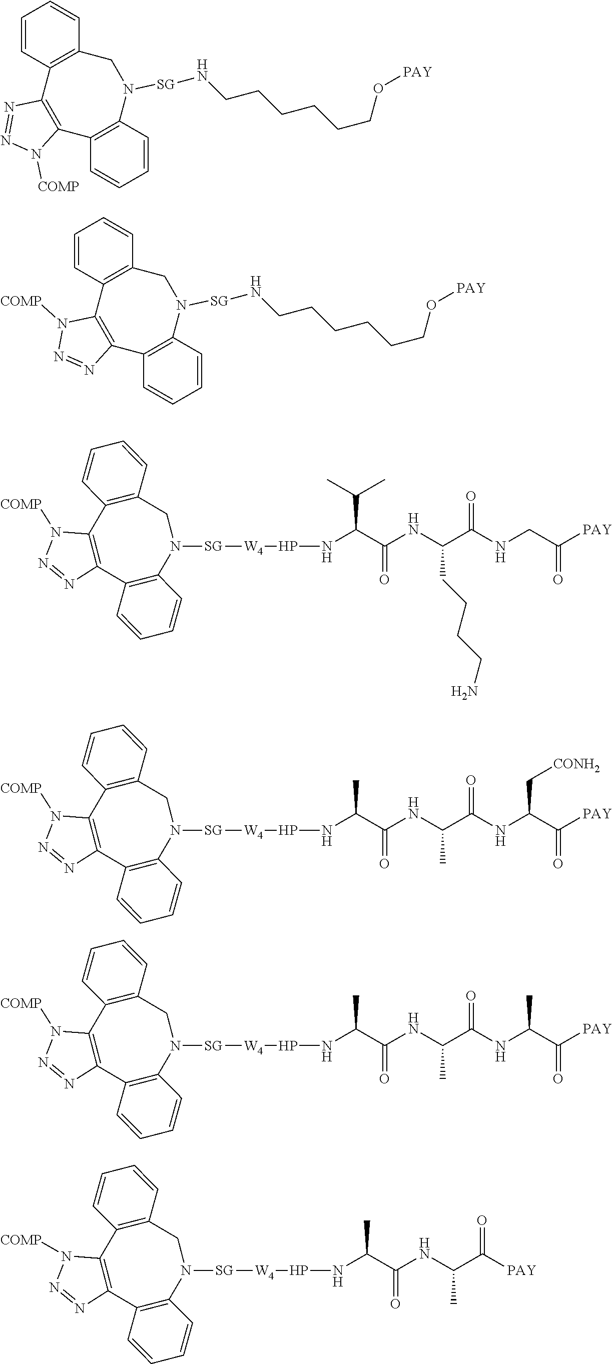

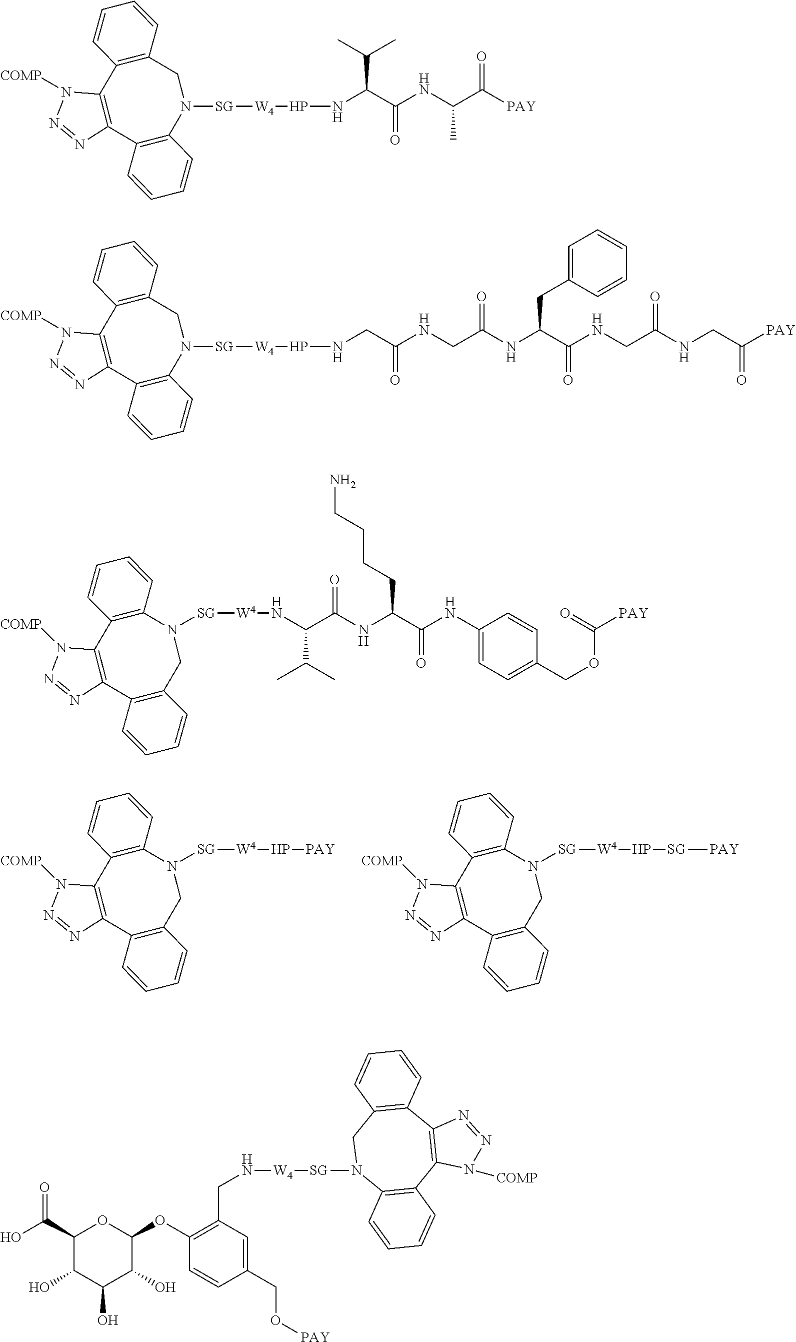

In an embodiment, provided herein is a conjugate according to any of the following formulas, where COMP indicates a residue of the anti-ROR1 antibody and PAY indicates a payload moiety:

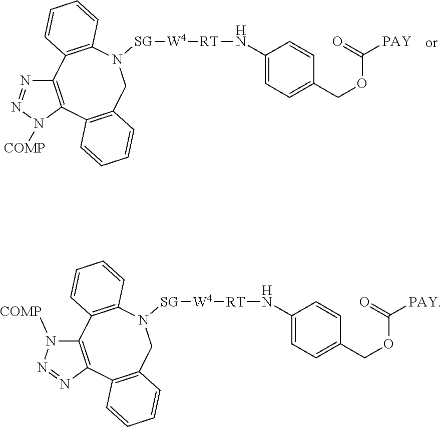

In an embodiment, provided herein is a conjugate according to any of the following formulas, where COMP indicates a residue of the anti-ROR1 antibody and PAY indicates a payload moiety:

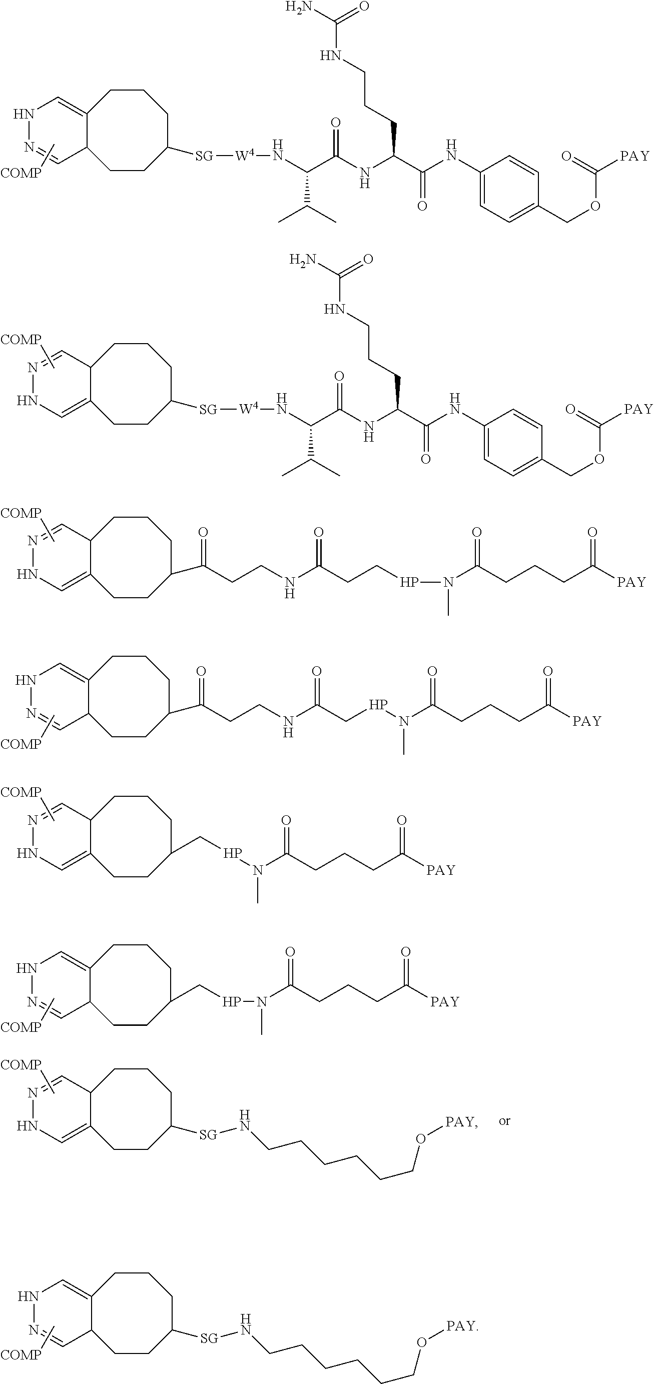

In an embodiment, provided herein is a conjugate according to any of Formulas 101a-104b, where COMP indicates a residue of the anti-ROR1 antibody and PAY indicates a payload moiety:

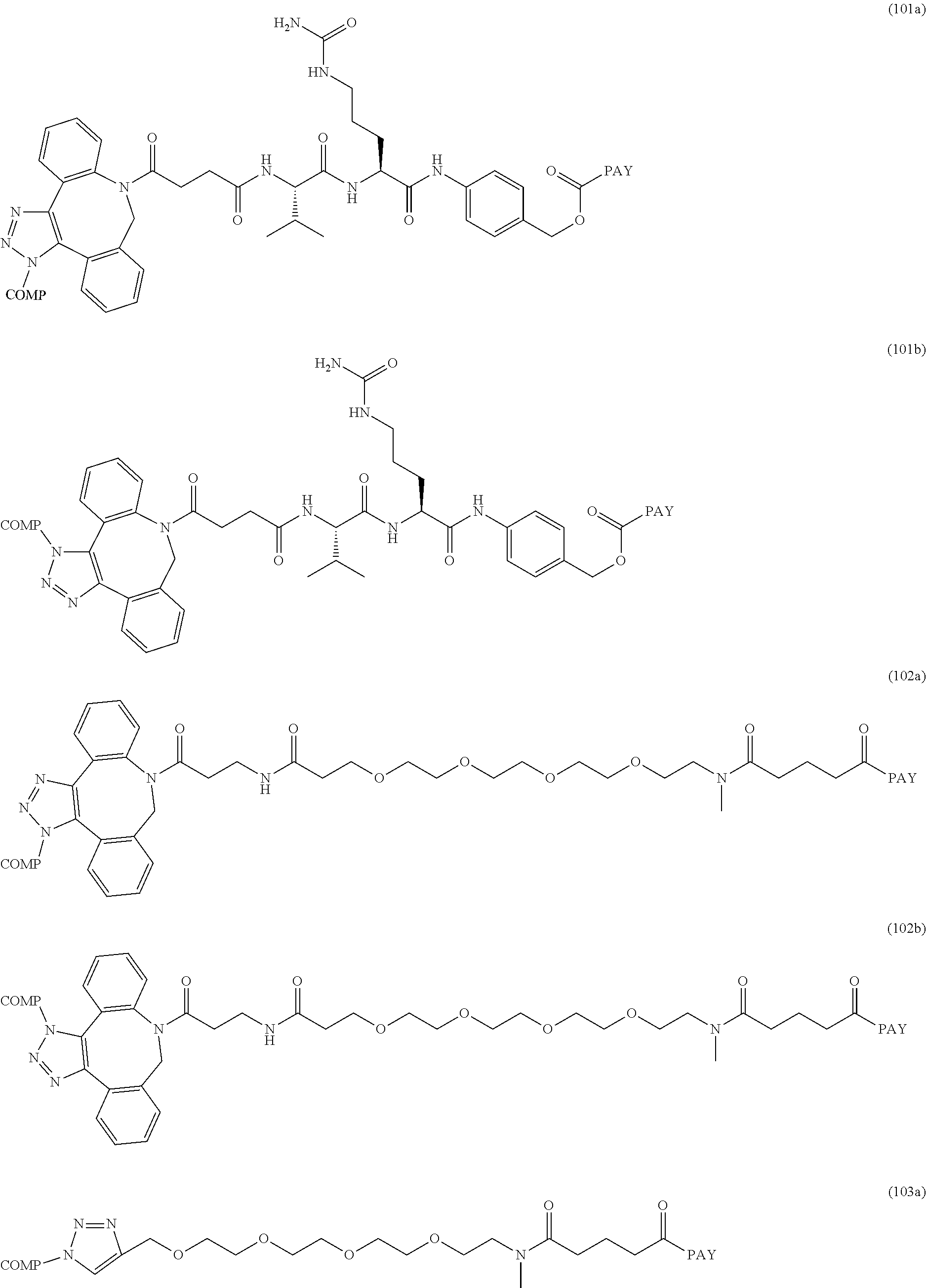

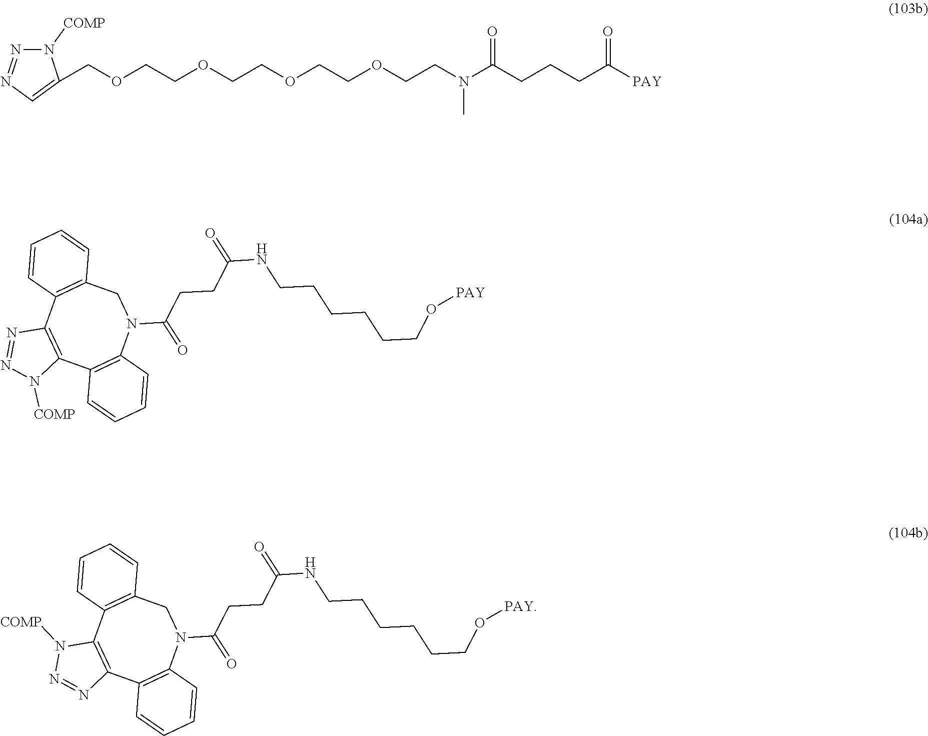

In any of the foregoing embodiments, the conjugate comprises n2 number of PAY moieties, wherein n2 is an integer from 1 to 10. In some embodiments, n2 is 2. In some embodiments, n2 is 3. In some embodiments, n2 is 4. In some embodiments, n2 is 5. In some embodiments, n2 is 6. In some embodiments, n2 is 7. In some embodiments, n2 is 8. In some embodiments, n2 is 9. In some embodiments, n2 is 10.

In some embodiments, provided herein are anti-ROR1 conjugates comprising a modified hemiasterlin and linker as described, for example, in PCT Publication No. WO 2016/123582. For example, the conjugate can have a structure comprising any of Formulas 1000-1000b, 1001-1001b, 1002-1002b, and I-XIXb-2, 101-111b, or 1-8b as described in PCT Publication No. WO 2016/2016/123582. Examples of conjugates comprising a modified hemiasterlin and linker are provided below.

In some embodiments, provided is a conjugate of Formula (I)

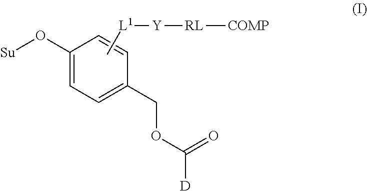

or a pharmaceutically acceptable salt thereof, wherein
COMP is a residue of an anti-ROR1 antibody provided herein;
L¹ is —C₁₋₆ alkylene-;
Y is —X¹—C₁₋₆ alkylene-[X¹—C₁₋₆ alkylene]ₙ-[X¹]ₚ—,
X¹—C₂₋₆ alkenylene-[X¹—C₂₋₆ alkenylene]ₙ-[X¹]ₚ—, —X¹—C₂₋₆ alkynylene-[X¹—C₂₋₆ alkynylene]ₙ-[X¹]ₚ—, wherein at least one alkylene, alkenylene or alkynylene in Y is substituted with one or more substituents selected from R⁵⁰; and
wherein the alkylene, alkenylene, or alkynylene in Y is optionally substituted with one or more substituents selected from R⁵¹;
R⁵⁰ is —C₁₋₆ alkylene-X²—[C₁₋₆ alkylene]ₘ-POLY,
C₂₋₆ alkenylene-X²—[C₂₋₆ alkenylene]ₘ-POLY, or —C₂₋₆ alkynylene-X²—[C₂₋₆ alkynylene]ₘ-POLY, wherein each alkylene, alkenylene or alkynylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
R⁵¹ is independently selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
X¹ and X² are independently selected from —C(O)— and —N(R¹⁰)C(O)—;
R¹⁰ is independently selected at each occurrence from hydrogen, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
POLY is a water-soluble polymer;
n is an integer selected from zero, one, two, and three;
m is an integer selected from zero and one;
p is an integer selected from zero and one;
Su is a hexose form of a monosaccharide;
D is a drug moiety; and
RL is a reactive group residue.

In some embodiments, provided is a conjugate of Formula (II)

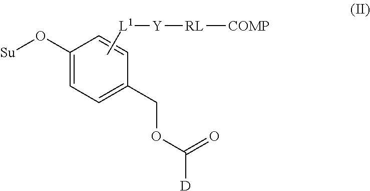

or a pharmaceutically acceptable salt thereof, wherein
COMP is a residue of an anti-ROR1 antibody provided herein;
L¹ is —C₁₋₆ alkylene-;
Y is —X¹—C₁₋₆ alkylene-[X¹—C₁₋₆ alkylene]ₙ-X¹—,
—X¹—C₂₋₆ alkenylene-[X¹—C₂₋₆ alkenylene]ₙ-X¹—, —X¹—C₂₋₆ alkynylene-[X¹—C₂₋₆ alkynylene]ₙ-X¹—, wherein at least one alkylene, alkenylene or alkynylene in Y is substituted with one or more substituents selected from R⁵⁰;
R⁵⁰ is —C₁₋₆ alkylene-X²—[C₁₋₆ alkylene]ₘ-POLY,
—C₂₋₆ alkenylene-X²—[C₂₋₆ alkenylene]ₘ-POLY, or —C₂₋₆ alkynylene-X²—[C₂₋₆ alkynylene]ₘ-POLY, wherein each alkylene, alkenylene or alkynylene of R⁵⁰ is optionally substituted with one or more substituents selected from halogen, —CN, —NO₂, —OH, —N(R¹⁰)₂, —C(O)N(R¹⁰)₂,
—C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
X¹ and X² are independently selected from —C(O)— and —N(R¹⁰)C(O)—;
R¹⁰ is independently selected at each occurrence from hydrogen, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
POLY is a water-soluble polymer;
n is an integer selected from zero, one, two, and three;
m is an integer selected from zero and one;
Su is a hexose form of a monosaccharide;
D is a drug moiety; and
RL is a reactive group residue.

In some embodiments, provided is a conjugate of Formula (IIA)

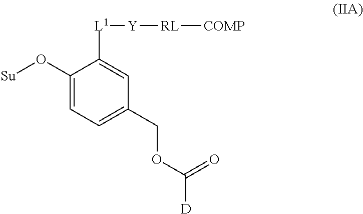

or a pharmaceutically acceptable salt thereof.

In some embodiments, the compound of Formula (II) is according to Formula (IIB)

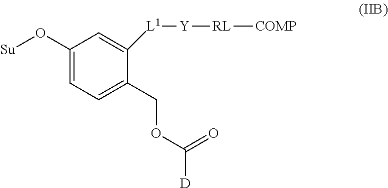

or a pharmaceutically acceptable salt thereof.

In some embodiments of Formula (I), (II), (IIA), or (IIB), L¹ is —C₁₋₃ alkylene-. In some embodiments, L¹ is —CH₂—. In some embodiments of Formula (I), (II), (IIA), or (IIB), L¹ is —CH₂CH₂—. In some embodiments, L¹ is —CH₂CH₂CH₂—.

In some embodiments of Formula (I), including any of the foregoing, p is 0. In some embodiments of Formula (I), including any of the foregoing, p is 1.

In some embodiments of Formula (I), (II), (IIA), or (IIB), including any of the foregoing, Y is —X¹—C₁₋₆ alkylene-[X¹—C₁₋₆ alkylene]ₙ-X¹, wherein at least one alkylene in Y is substituted with one or more substituents selected from R⁵⁰. In some embodiments of Formula (I), (II), (IIA), or (IIB), including any of the foregoing, Y is —X¹—C₁₋₆ alkylene-[X¹—C₁₋₆ alkylene]ₙ-, wherein at least one alkylene in Y is substituted with one or more substituents selected from R⁵⁰. In some embodiments of Formula (I), (II), (IIA), or (IIB), including any of the foregoing, Y is —X¹—C₂₋₆ alkenylene-[X¹—C₂₋₆ alkenylene]ₙ-X¹— wherein at least one alkenylene in Y is substituted with one or more substituents selected from R⁵⁰. In some embodiments of Formula (I), (II), (IIA), or (IIB), including any of the foregoing, Y is —X¹—C₂₋₆ alkenylene-[X¹—C₂₋₆ alkenylene]ₙ- wherein at least one alkenylene in Y is substituted with one or more substituents selected from R⁵⁰. In some embodiments of Formula (I), (II), (IIA), or (IIB), including any of the foregoing, Y is —X¹—C₂₋₆ alkynylene-[X¹—C₂₋₆ alkynylene]ₙ-X¹— wherein at least one alkynylene in Y is substituted with one or more substituents selected from R⁵⁰. In some embodiments of Formula (I), (II), (IIA), or (IIB), including any of the foregoing, Y is —X¹—C₂₋₆ alkynylene-[X¹—C₂₋₆ alkynylene]ₙ- wherein at least one alkynylene in Y is substituted with one or more substituents selected from R⁵⁰. In some embodiments of Formula (I), (II), (IIA), or (IIB), including any of the foregoing, n is zero. In some embodiments of Formula (I), (II), (IIA), or (IIB), n is one. In some embodiments of Formula (I), (II), (IIA), or (IIB), including any of the foregoing, n is two. In some embodiments, including any of the foregoing, n is three.

In certain embodiments of Formula (I), (II), (IIA), or (IIB), including any of the foregoing, Y is —X¹—C₁₋₄ alkylene-[X¹—C₁₋₄ alkylene]ₙ-X¹—, wherein at least one alkylene in Y is substituted with one or more substituents selected from R⁵⁰. In some embodiments of Formula (I), (II), (IIA), or (IIB), including any of the foregoing, n is zero. In some embodiments of Formula (I), (II), (IIA), or (IIB), including any of the foregoing, n is one. In some embodiments of Formula (I), (II), (IIA), or (IIB), including any of the foregoing, n is two. In some embodiments of Formula (I), (II), (IIA), or (IIB), including any of the foregoing, n is three.

In certain embodiments of Formula (I), (II), (IIA), or (IIB), including any of the foregoing, Y is —X¹—C₁₋₄ alkylene-X¹—C₁₋₄ alkylene-X¹—C₁₋₄ alkylene-X¹—C₁₋₄ alkylene-X¹—, wherein at least one alkylene in Y is substituted with one or more substituents selected from R⁵⁰. In certain embodiments of Formula (I), (II), (IIA), or (IIB), including any of the foregoing, Y is —X¹—C₁₋₄ alkylene-X¹—C₁₋₄ alkylene-X¹—C₁₋₄ alkylene-X¹—, wherein at least one alkylene in Y is substituted with one or more substituents selected from R⁵⁰. In certain embodiments of Formula (I), (II), (IIA), or (IIB), including any of the foregoing, Y is —X¹—C₁₋₄ alkylene-X¹—C₁₋₄ alkylene-X¹—, wherein at least one alkylene in Y is substituted with one or more substituents selected from R⁵⁰.

In certain embodiments of Formula (I), (II), (IIA), or (IIB), including any of the foregoing, R⁵⁰ is —C₁₋₆ alkylene-X²—[C₁₋₆ alkylene]ₘ-POLY, wherein each alkylene of R⁵⁰ is optionally substituted with one or more substituents selected from halogen, —CN, —NO₂, —OH,

—N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, R⁵⁰ is —C₁₋₄ alkylene-X²—[C₁₋₄ alkylene]ₘ-POLY, wherein each alkylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, each alkylene of R⁵⁰ is optionally substituted with one or more substituents selected from halogen, —OH, —N(R¹⁰)₂, —C(O)N(R¹⁰)₂, —C(O)—, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, m is zero. In some embodiments of Formula (I), (II), (IIA), or (IIB), including any of the foregoing, m is one.

In certain embodiments of Formula (I), (II), (IIA), or (IIB), including any of the foregoing, R⁵⁰ is —C₂₋₆ alkenylene-X²—[C₂₋₆ alkenylene]ₘ-POLY, wherein each alkenylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In some embodiments of Formula (I), (II), (IIA), or (IIB), including any of the foregoing, m is zero. In some embodiments of Formula (I), (II), (IIA), or (IIB), including any of the foregoing, m is one.

In certain embodiments of Formula (I), (II), (IIA), or (IIB), including any of the foregoing, R⁵⁰ is —C₂₋₆ alkynylene-X²—[C₂₋₆ alkynylene]ₘ-POLY, wherein each alkynylene of R⁵⁰ is optionally substituted with one or more substituents selected from halogen, —CN, —NO₂, —OH,

—N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl. In come embodiments of Formula (I), (II), (IIA), or (IIB), including any of the foregoing, m is zero. In some embodiments of Formula (I), (II), (IIA), or (IIB), including any of the foregoing, m is one.

In certain embodiments of Formula (I), (II), (IIA), or (IIB), including any of the foregoing, POLY is polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(□-hydroxy acid), poly(vinyl alcohol), polyphosphazene, polyoxazolines (POZ), poly(N-acryloylmorpholine), polysarcosine, or a combination thereof. In some embodiments of Formula (I), (II), (IIA), or (IIB), including any of the foregoing, POLY is polyethylene glycol (PEG). In some embodiments of Formula (I), (II), (IIA), or (IIB), including any of the foregoing, POLY is methoxypolyethylene glycol (mPEG). In some embodiments of Formula (I), (II), (IIA), or (IIB), including any of the foregoing, POLY is poly(propylene glycol) (PPG). In some embodiments, POLY is copolymers of ethylene glycol and propylene glycol. In some embodiments of Formula (I), (II), (IIA), or (IIB), including any of the foregoing, POLY is poly(oxyethylated polyol). In some embodiments of Formula (I), (II), (IIA), or (IIB), including any of the foregoing, POLY is poly(olefinic alcohol). In some embodiments, POLY is poly(vinylpyrrolidone). In some embodiments of Formula (I), (II), (IIA), or (IIB), including any of the foregoing, POLY is poly(hydroxyalkylmethacrylamide). In some embodiments of Formula (I), (II), (IIA), or (IIB), including any of the foregoing, POLY is poly(hydroxyalkylmethacrylate). In some embodiments of Formula (I), (II), (IIA), or (IIB), including any of the foregoing, POLY is poly(saccharides). In some embodiments of Formula (I), (II), (IIA), or (IIB), including any of the foregoing, POLY is poly(□-hydroxy acid). In some embodiments of Formula (I), (II), (IIA), or (IIB), including any of the foregoing, POLY is poly(vinyl alcohol). In some embodiments of Formula (I), (II), (IIA), or (IIB), including any of the foregoing, POLY is polyphosphazene. In some embodiments of Formula (I), (II), (IIA), or (IIB), including any of the foregoing, POLY is polyoxazolines (POZ). In some embodiments of Formula (I), (II), (IIA), or (IIB), including any of the foregoing, POLY is poly(N-acryloylmorpholine). In some embodiments of Formula (I), (II), (IIA), or (IIB), including any of the foregoing, POLY is polysarcosine. In some embodiments of Formula (I), (II), (IIA), or (IIB), including any of the foregoing, POLY is a nonpeptidic, water-soluble polymer. In certain embodiments of Formula (I), (II), (IIA), or (IIB), including any of the foregoing, POLY includes a polyethylene glycol (PEG) or methoxypolyethylene glycol (mPEG). In certain embodiments of Formula (I), (II), (IIA), or (IIB), including any of the foregoing, POLY is

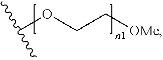

wherein

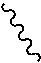

represents attachment to the remainder of the compound, and wherein n1 is an integer from one to twenty. In certain embodiments of Formula (I), (II), (IIA), or (IIB), including any of the foregoing, n1 is an integer between five to fifteen. In some embodiments of Formula (I), (II), (IIA), or (IIB), including any of the foregoing, n1 is one. In some embodiments of Formula (I), (II), (IIA), or (IIB), including any of the foregoing, n1 is two. In some embodiments of Formula (I), (II), (IIA), or (IIB), including any of the foregoing, n1 is three. In some embodiments of Formula (I), (II), (IIA), or (IIB), including any of the foregoing, n1 is four. In some embodiments of Formula (II), (IIA), or (IIB), including any of the foregoing, n1 is five. In some embodiments of Formula (I), (II), (IIA), or (IIB), including any of the foregoing, n1 is six. In some embodiment of Formula (I), (II), (IIA), or (IIB) s, n1 is seven. In some embodiments of Formula (I), (II), (IIA), or (IIB), including any of the foregoing, n1 is eight. In some embodiments of Formula (I), (II), (IIA), or (IIB), including any of the foregoing, n1 is nine. In some embodiments of Formula (I), (II), (IIA), or (IIB), including any of the foregoing, n1 is ten. In some embodiments of Formula (I), (II), (IIA), or (IIB), including any of the foregoing, n1 is eleven. In some embodiments of Formula (I), (II), (IIA), or (IIB), including any of the foregoing, n1 is twelve. In some embodiments of Formula (I), (II), (IIA), or (IIB), including any of the foregoing, n1 is thirteen. In some embodiments of Formula (I), (II), (IIA), or (IIB), including any of the foregoing, n1 is fourteen. In some embodiments of Formula (I), (II), (IIA), or (IIB), including any of the foregoing, n1 is fifteen. In some embodiments of Formula (I), (II), (IIA), or (IIB), including any of the foregoing, n1 is sixteen. In some embodiments of Formula (I), (II), (IIA), or (IIB), including any of the foregoing, n1 is seventeen. In some embodiments of Formula (I), (II), (IIA), or (IIB), n1 is eighteen. In some embodiments of Formula (I), (II), (IIA), or (IIB), including any of the foregoing, n1 is nineteen. In some embodiments of Formula (II), (IIA), or (IIB), including any of the foregoing, n1 is twenty. In some embodiments of Formula (I), (II), (IIA), or (IIB), including any of the foregoing, n1 is twenty-one. In some embodiments of Formula (I), (II), (IIA), or (IIB), including any of the foregoing, n1 is twenty-two. In some embodiments of Formula (I), (II), (IIA), or (IIB), including any of the foregoing, n1 is twenty-three. In some embodiments of Formula (I), (II), (IIA), or (IIB), including any of the foregoing, n1 is twenty-four. In some embodiments of Formula (I), (II), (IIA), or (IIB), including any of the foregoing, n1 is twenty-five. In some embodiments of Formula (I), (II), (IIA), or (IIB), including any of the foregoing, n1 is twenty-six. In some embodiments of Formula (I), (II), (IIA), or (IIB), including any of the foregoing, n1 is twenty-seven. In some embodiments of Formula (I), (II), (IIA), or (IIB), including any of the foregoing, n1 is twenty-eight. In some embodiments of Formula (I), (II), (IIA), or (IIB), including any of the foregoing, n1 is twenty-nine. In some embodiments of Formula (I), (II), (IIA), or (IIB), including any of the foregoing, n1 is thirty.

In certain embodiments, RL includes an alkyne, cyclooctyne, a strained alkene, a tetrazine, an amine, methylcyclopropene, a thiol, a para-acetyl-phenylalanine residue, an oxyamine, a maleimide, or an azide. In some embodiments, RL includes an alkyne. In some embodiments, RL includes an cyclooctyne. In some embodiments, RL includes a strained alkene. In some embodiments, RL includes a tetrazine. In some embodiments, RL includes an amine. In some embodiments, RL includes an methylcyclopropene. In some embodiments, RL includes a thiol. In some embodiments, RL includes a para-acetyl-phenylalanine residue. In some embodiments, RL includes an oxyamine. In some embodiments, RL includes a maleimide. In some embodiments, RL includes an azide. In certain embodiments, RL is selected from the group consisting of

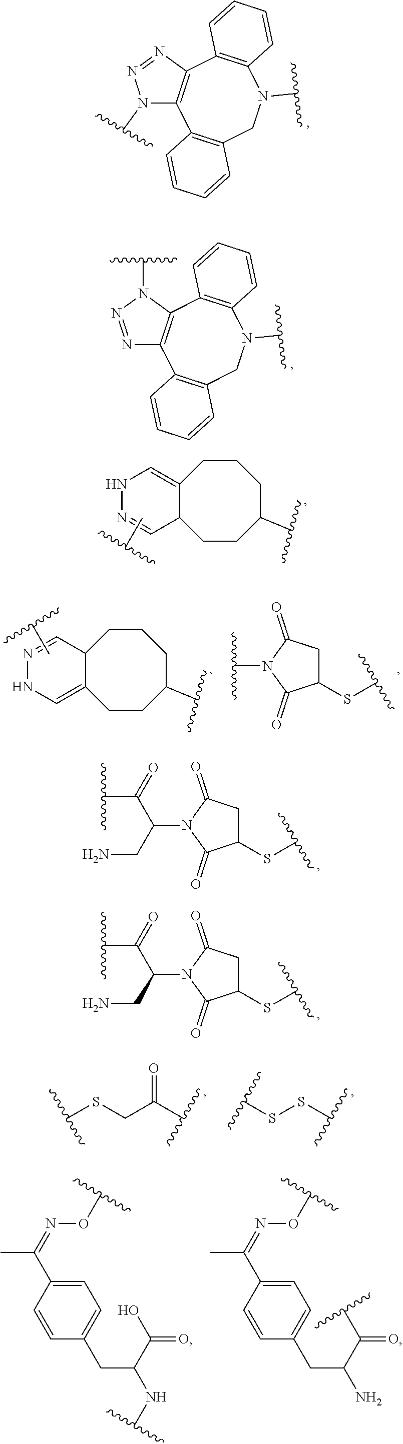

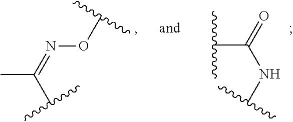

represents attachment to the remainder of the compound. In some embodiments, RL is

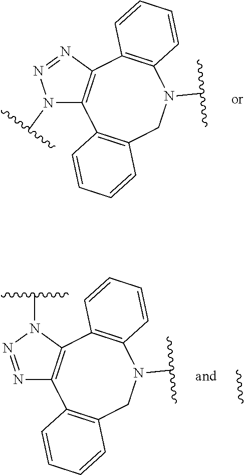

represents attachment to the remainder of the compound. In one some embodiments, RL is

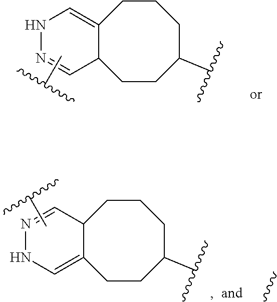

represents attachment to the remainder of the compound. In some embodiments, RL is

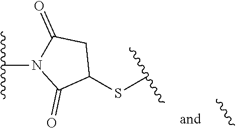

represents attachment to the remainder of the compound. In some embodiments, RL is

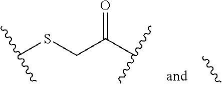

represents attachment to the remainder of the compound. In some embodiments, RL is

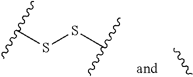

represents attachment to the remainder of the compound. In some embodiments, RL is

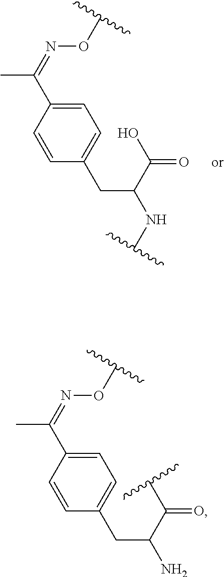 [SMILES: *NC(Cc1ccc(/C(C)=N\O*)cc1)C(=O)O ; "or" ; *O/N=C(/C)c1ccc(CC(N)C(*)=O)cc1]

wherein

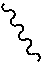

represents attachment to the remainder of the compound. In some embodiments, RL is

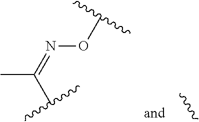

represents attachment to the remainder of the compound. In some embodiments, RL is

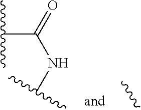

represents attachment to the remainder of the compound. In some embodiments, RL is

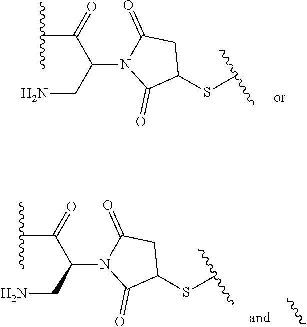

represents attachment to the remainder of the compound. In some embodiments, Su is a sugar moiety. In some embodiments, Su is a hexose form of a monosaccharide. Su may be a glucuronic acid or mannose residue. In certain embodiments, Su is

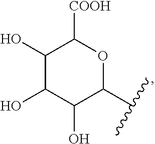 [SMILES: *C1OC(C(=O)O)C(O)C(O)C1O]

wherein

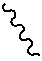

represents attachment to the remainder of the compound. In certain embodiments, Su is

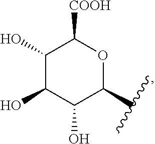 [SMILES: *[C@@H]1O[C@H](C(=O)O)[C@@H](O)[C@H](O)[C@H]1O]

wherein

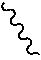

represents attachment to the remainder of the compound.

In one aspect, provided herein is a conjugate of Formula (III):

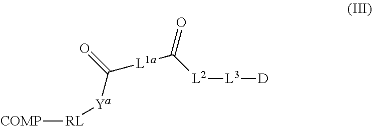

or a pharmaceutically acceptable salt and/or regioisomer thereof;
wherein
L^{1a} is selected from

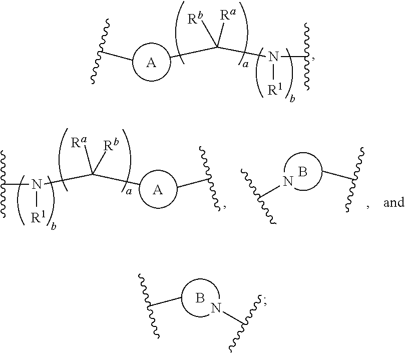

Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—,
—C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y^{a} is *—C(O)—(CR^{a}R^{b})_{c}—NH— or *—C(O)—(CR^{a}R^{b})_{c}—, wherein * represents where Y^{a} is bound to RL;
c is an integer selected from 1, 2, 3, 4, 5, or 6;
RL is a reactive group residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-L⁴-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ—Z—(CR^{a}R^{b})—Z-AA-C(O)—, —C(O)O-L⁴-Z—C(O)—(CR^{a}R^{b})ₐ—Z—C(O)—, -AA-Z—, or —(CR^{a}R^{b})ₐ—Z-AA-C(O)—;
Z is selected from —NR²— and —O—;
AA is an amino acid residue or a peptide residue;
L⁴ is

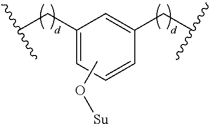

wherein Su is a hexose form of a monosaccharide;
d is an integer independently selected from 1, 2, and 3;
D is a drug moiety;
COMP is a residue of a ROR1 antibody; and

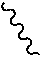

represents attachment to the remainder of the compound.

In certain embodiments, the compound of Formula (III) is a compound of Formula (IIIA):

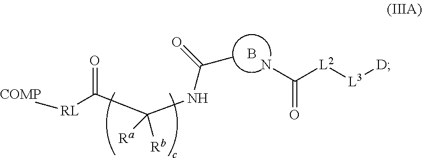

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (IIIA) is selected from the following:

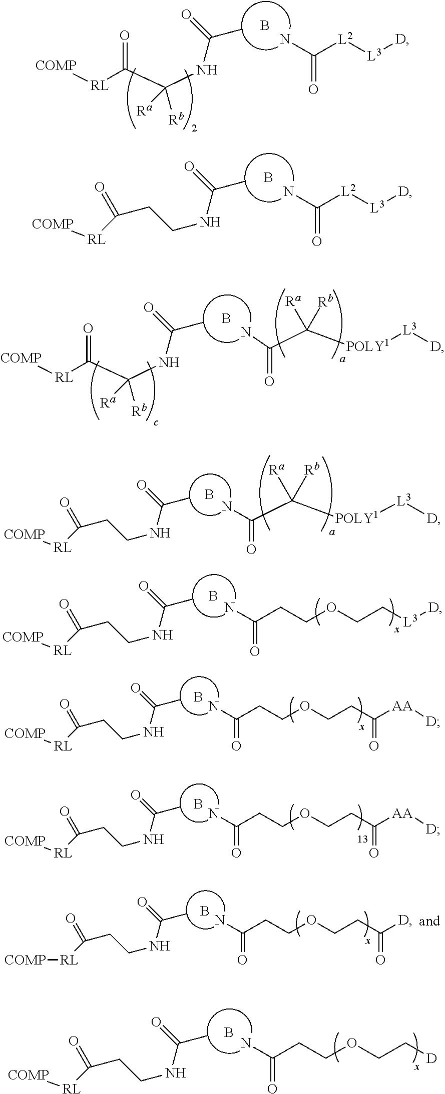

or a pharmaceutically acceptable salt and/or regioisomer thereof.

In certain embodiments, the compound of Formula (III) is a compound of Formula (IIIB):

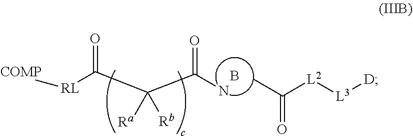

or a pharmaceutically acceptable salt and/or regioisomer thereof;
wherein integer c, RL, R^{a}, R^{b}, Ring B, L², L³, D, and COMP are as defined herein.

In certain embodiments, the compound of Formula (IIIB) is selected from the following:

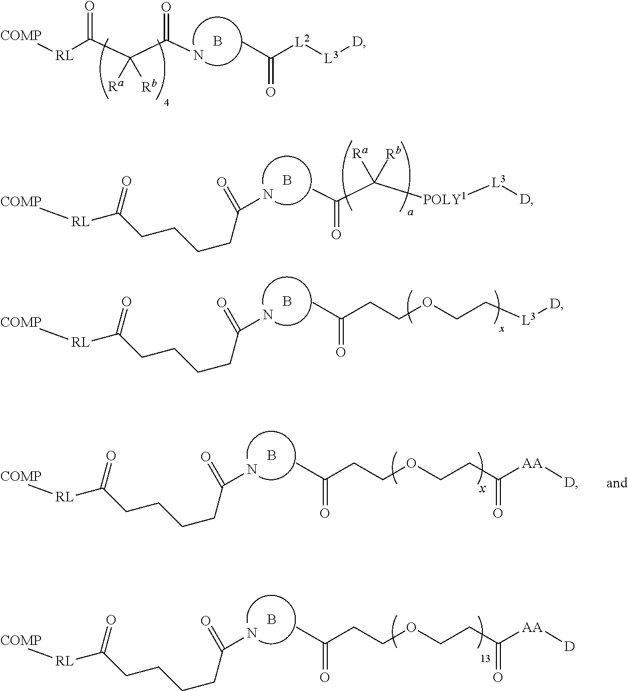

or a pharmaceutically acceptable salt and/or regioisomer thereof.

In certain embodiments, the compound of Formula (III) is a compound of Formula (IIIC):

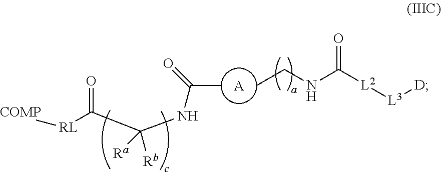

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (IIIC) is selected from the following:

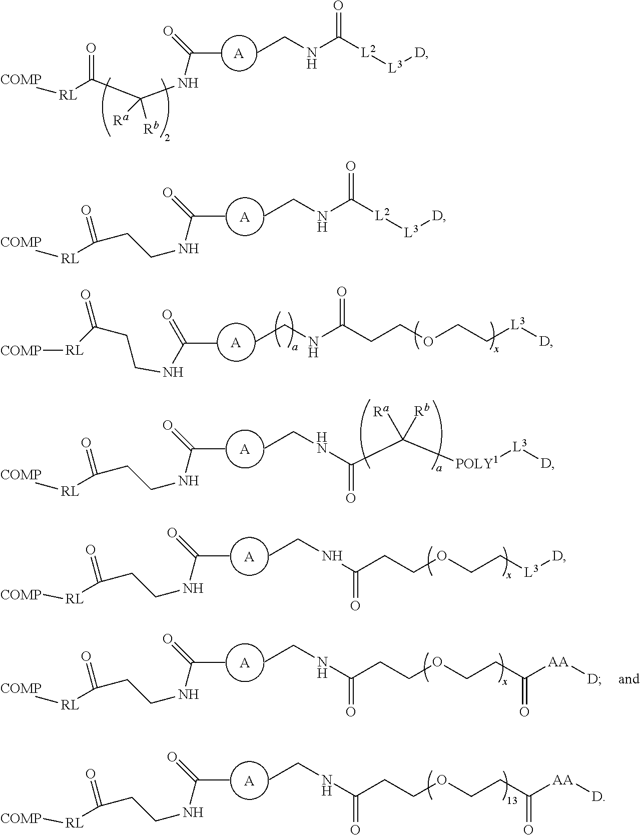

Or a pharmaceutically acceptable salt and/or regioisomer thereof.

In certain embodiments, the compound of Formula (III) is a compound of Formula (IIID):

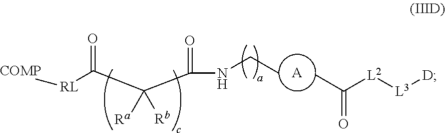

or a pharmaceutically acceptable salt and/or regioisomer thereof;
wherein integer a, integer c, RL, R^{a}, R^{b}, Ring A, L², L³, D, and COMP are as defined herein.

In certain embodiments, the compound of Formula (IIID) is selected from the following:

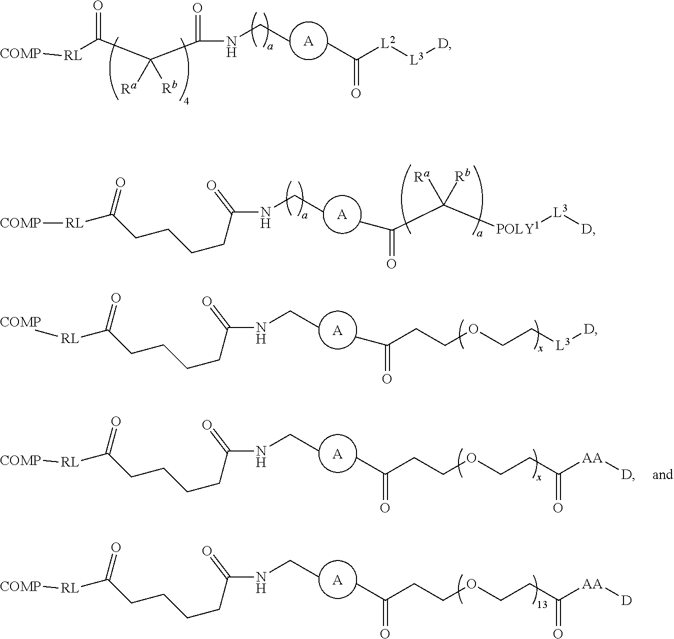

or a pharmaceutically acceptable salt and/or regioisomer thereof.

In certain embodiments, the compound of Formula (III) is a compound of Formula (IIIE):

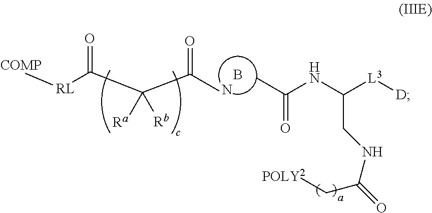

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (IIIE) is selected from the following:

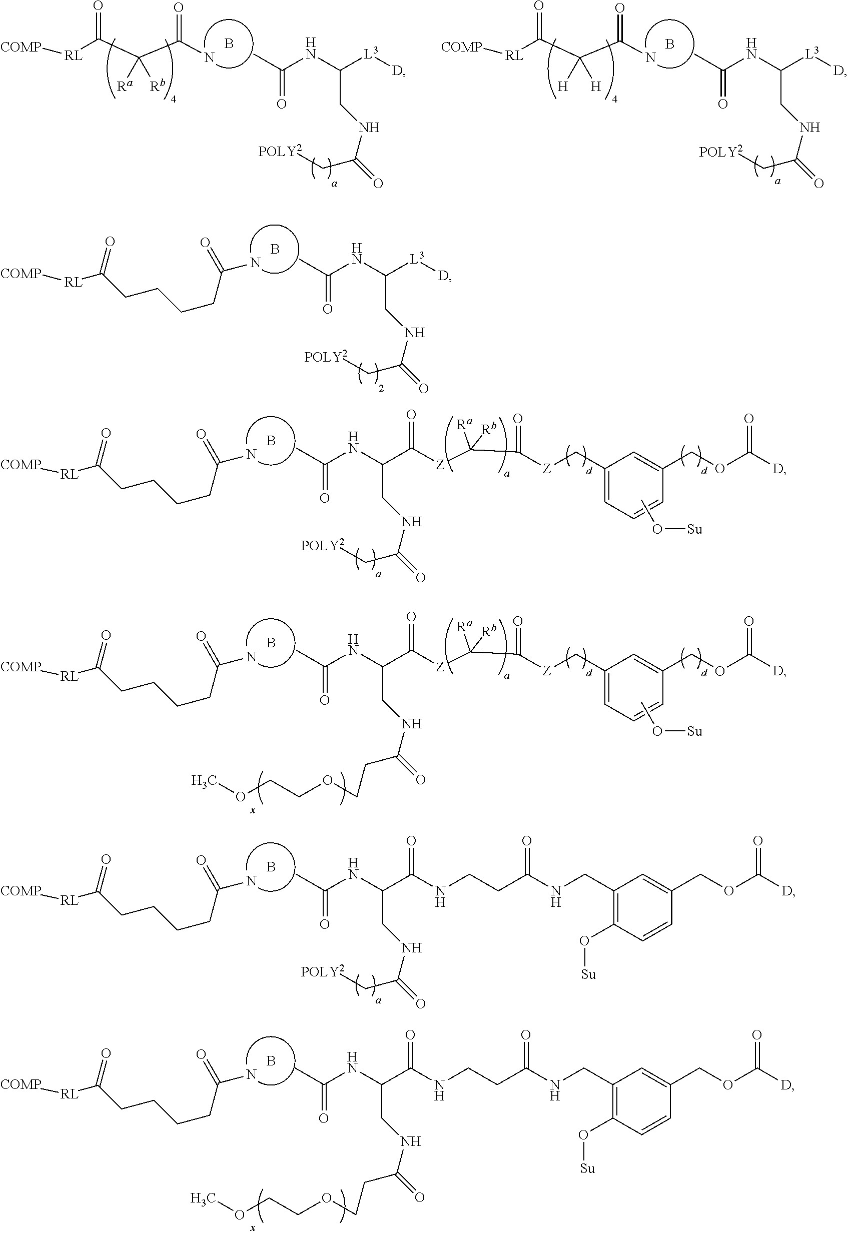

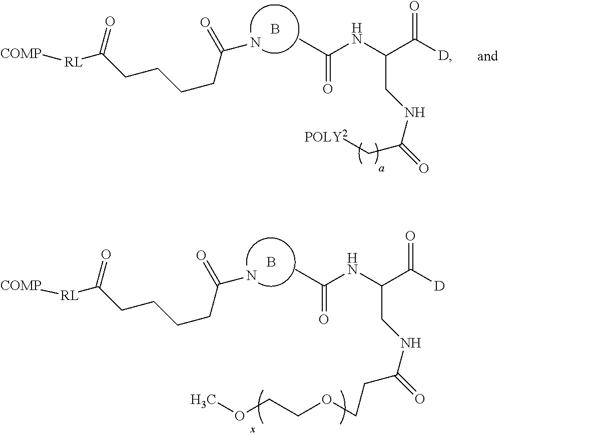

or a pharmaceutically acceptable salt and/or regioisomer thereof.

In certain embodiments, the compound of Formula (III) is a compound of Formula (IIIF):

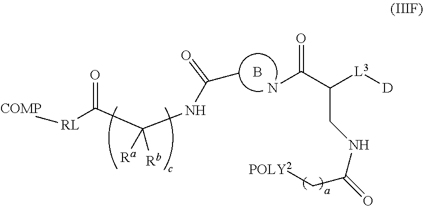

or a pharmaceutically acceptable salt and/or regioisomer thereof;
wherein integer a, integer c, RL, R^{a}, R^{b}, Ring B, L³, POLY², D, and COMP are as defined herein.

In certain embodiments, the compound of Formula (IIIF) is selected from the following:

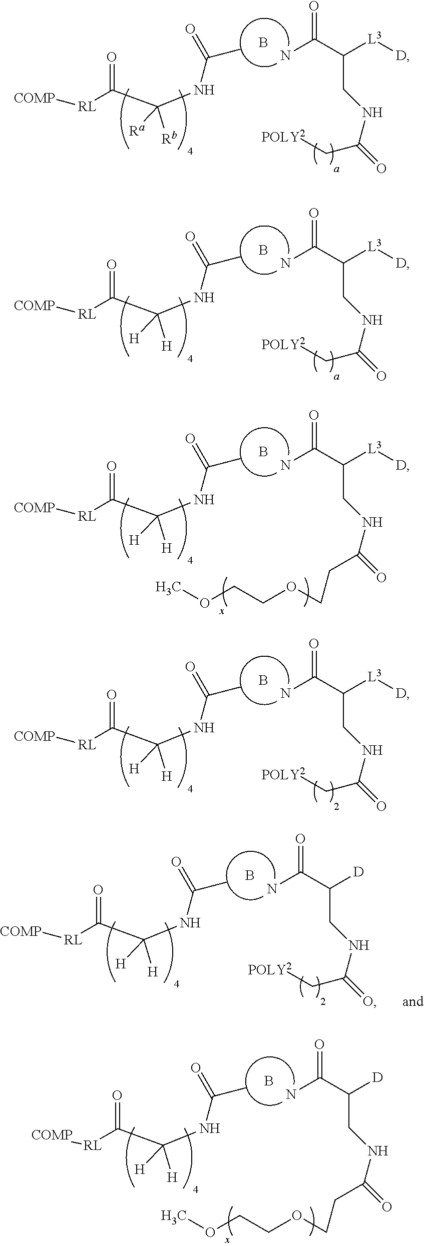

or a pharmaceutically acceptable salt and/or regioisomer thereof.

In certain embodiments, the compound of Formula (III) is a compound of Formula (IIIG):

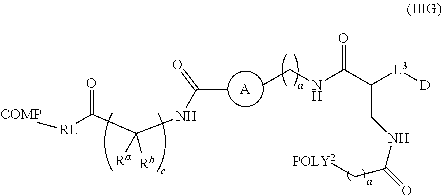

or a pharmaceutically acceptable salt and/or regioisomer thereof;
wherein integer a, integer c, RL, R^{a}, R^{b}, Ring A, L³, POLY², D, and COMP are as defined herein.

In certain embodiments, the compound of Formula (IIIG) is selected from the following:

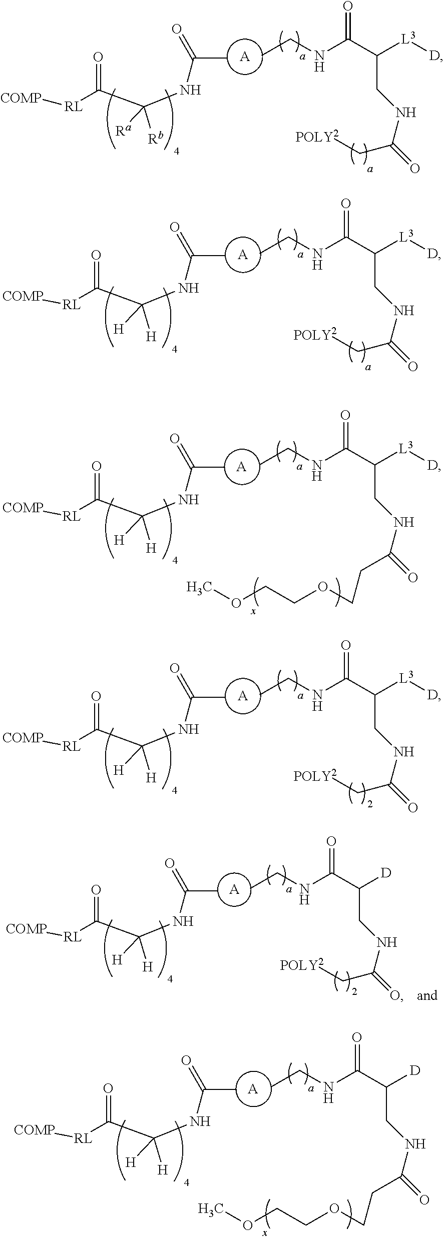

or a pharmaceutically acceptable salt and/or regioisomer thereof.

In certain embodiments, the compound of Formula (III) is a compound of Formula (IIIH):

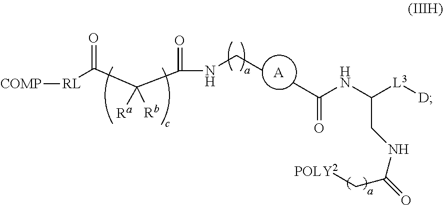

or a pharmaceutically acceptable salt and/or regioisomer thereof;
wherein integer a, integer c, RL, R^{a}, R^{b}, Ring A, L³, POLY², D, and COMP are as defined herein.

In certain embodiments, the compound of Formula (IIIH) is selected from the following:

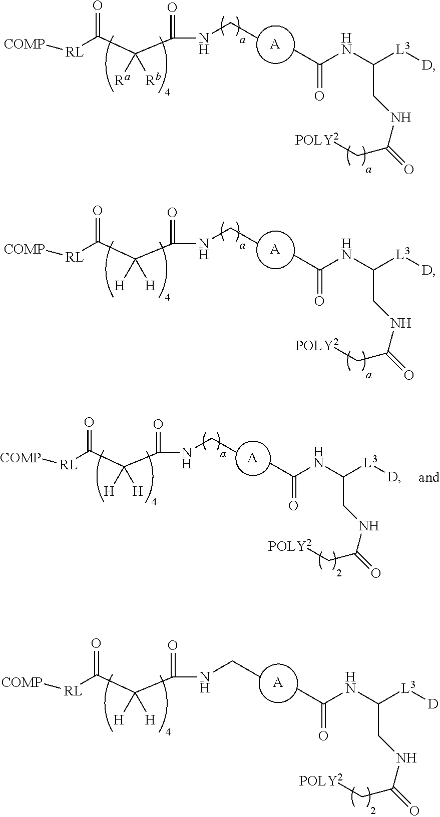

or a pharmaceutically acceptable salt and/or regioisomer thereof.

In one aspect, provided herein is a conjugate of Formula (V):

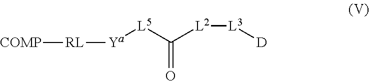

or a pharmaceutically acceptable salt and/or regioisomer thereof;
wherein
L⁵ is a linker comprising an unnatural amino acid; and
RL, COMP, Y^{a}, L², L³, and D are as defined herein.

In certain embodiments, the compound of Formula (V) is a compound of Formula (VA):

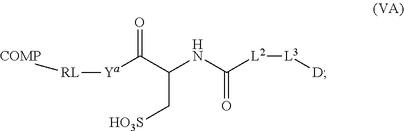

or a pharmaceutically acceptable salt and/or regioisomer thereof;
wherein RL, COMP, Y^{a}, L², L³, and D are as defined herein.

In certain embodiments, the compound of Formula (V) is a compound of Formula (VB):

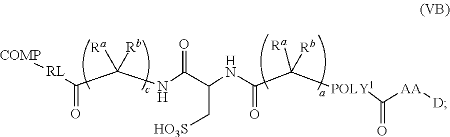

or a pharmaceutically acceptable salt and/or regioisomer thereof;
wherein integer a, integer c, RL, COMP, R^{a}, R^{b}, POLY¹, AA, and D are as defined herein.

In certain embodiments, the compound of Formula (VB) is a compound of the formula:

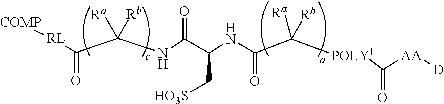

or a pharmaceutically acceptable salt and/or regioisomer thereof.

In certain embodiments of Formula (III)-(VB), including any of the foregoing, L^{1a} is

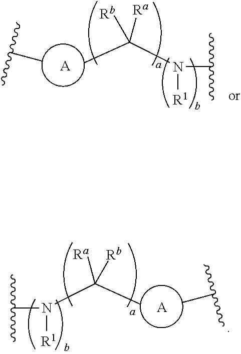

In certain embodiments of Formula (III)-(VB), including any of the foregoing, L^{1a} is

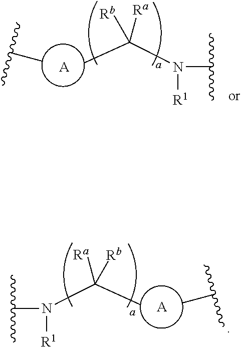

In certain embodiments of Formula (III)-(VB), including any of the foregoing, L^{1a} is

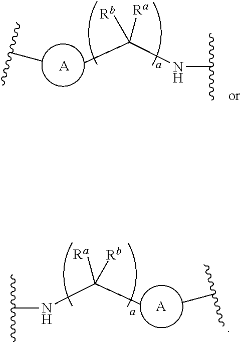

In certain embodiments of Formula (III)-(VB), including any of the foregoing, L^{1a} is

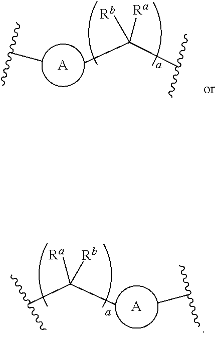

In certain embodiments of Formula (III)-(VB), including any of the foregoing, L^{1a} is

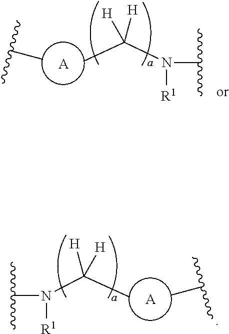

In certain embodiments of Formula (III)-(VB), including any of the foregoing, L^{1a} is

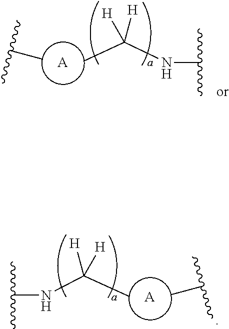

In certain embodiments of Formula (III)-(VB), including any of the foregoing, L^{1a} is

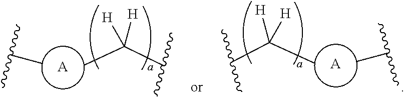

In certain embodiments of Formula (III)-(VB), including any of the foregoing, L^{1a} is

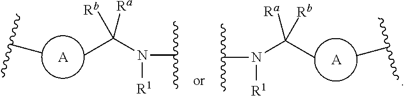

In certain embodiments of Formula (III)-(VB), including any of the foregoing, L^{1a} is

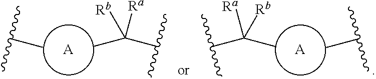

In certain embodiments of Formula (III)-(VB), including any of the foregoing, L^{1a} is

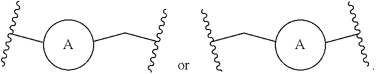

In certain embodiments of Formula (III)-(VB), including any of the foregoing, L^{1a} is

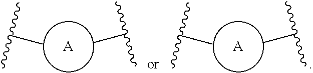

In certain embodiments of Formula (III)-(VB), including any of the foregoing, L^{1a} is

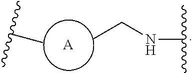

In certain embodiments of Formula (III)-(VB), including any of the foregoing, L^{1a} is

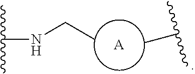

In certain embodiments of Formula (III)-(VB), including any of the foregoing, L^{1a} is

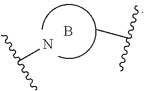

In certain embodiments of Formula (III)-(VB), including any of the foregoing, L^{1a} is

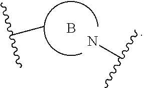

In certain embodiments of Formula (III)-(VB), including any of the foregoing, Ring B of L^{1a} is an optionally substituted 5- to 12-membered N-linked bridged, fused, or spirocyclic bicyclic heterocycle containing 1, 2, or 3 heteroatoms independently selected from N, O, and S including the N to which the ring is attached, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl. In certain embodiments of Formula (III)-(VB), including any of the foregoing, Ring B of L^{1a} is an optionally substituted 5- to 12-membered N-linked spirocyclic bicyclic heterocycle containing 1, 2, or 3 heteroatoms independently selected from N, O, and S including the N to which the ring is attached.

In certain embodiments of Formula (III)-(VB), including any of the foregoing, Ring B of L^{1a} is selected from

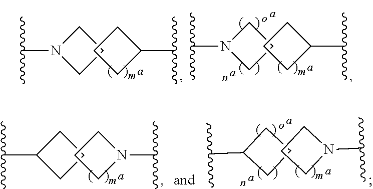

wherein m^{a} is an integer selected from 1, 2, 3, 4, and 5; and
each of n^{a} and o^{a} is an integer independently selected from 1, 2, and 3.

In certain embodiments of Formula (III)-(VB), including any of the foregoing, Ring B of L^{1a} is

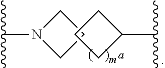

In certain embodiments of Formula (III)-(VB),

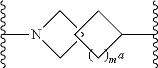

is selected from

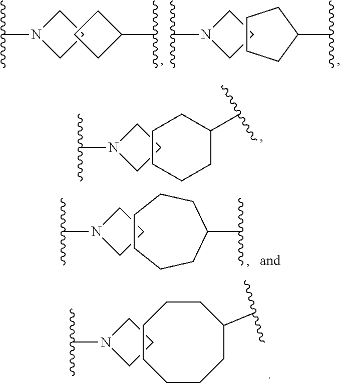

In certain embodiments of Formula (III)-(VB),

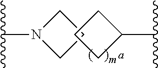

is selected from

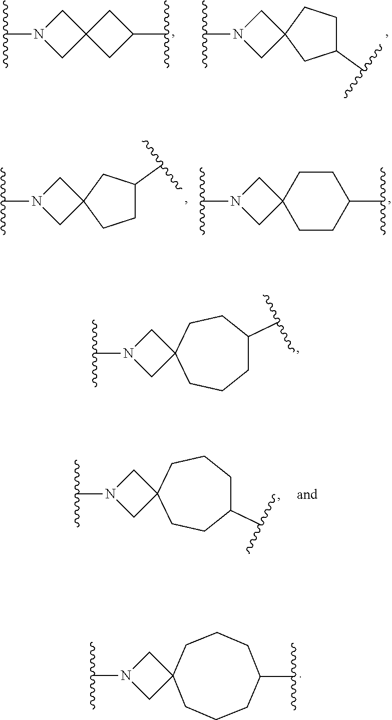

In certain embodiments of Formula (III)-(VB), Ring B of L^{1a} is

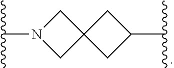 [SMILES: *C1CC2(C1)CN(*)C2]

In certain embodiments, Ring B is

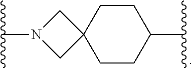 [SMILES: *C1CCC2(CC1)CN(*)C2]

In certain embodiments of Formula (III)-(VB), including any of the foregoing, Ring B of L^{1a} is

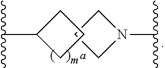

In certain embodiments of Formula (III)-(VB),

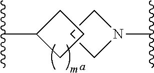

is selected from

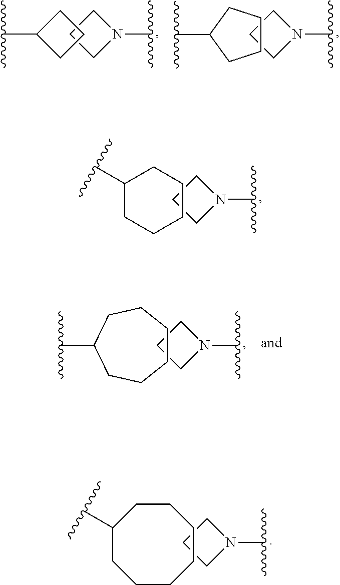

In certain embodiments of Formula (III)-(VB),

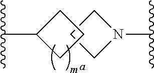

is selected from

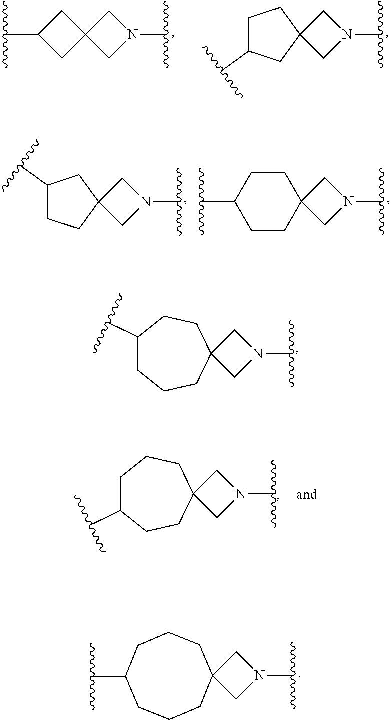

In certain embodiments of Formula (III)-(VB), Ring B of L^{1a} is

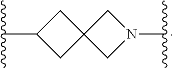 [SMILES: *C1CC2(C1)CN(*)C2]

In certain embodiments of Formula (III)-(VB), Ring B of L^{1a} is

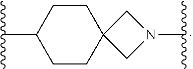 [SMILES: *C1CCC2(CC1)CN(*)C2]

In certain embodiments of Formula (III)-(VB), including any of the foregoing, Ring B of L^{1a} is

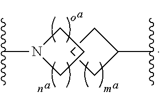

In certain embodiments of Formula (III)-(VB),

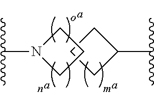

is

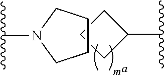

wherein m^{a} is 1, 2, or 3. In certain embodiments of Formula (III)-(VB),

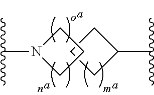

is

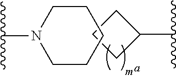

wherein m^{a} is 1, 2, or 3. In certain embodiments of Formula (III)-(VB),

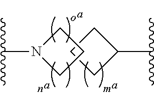

is

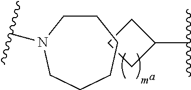

wherein m^{a} is 1, 2, or 3. In certain embodiments of Formula (III)-(VB),

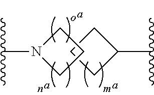

is

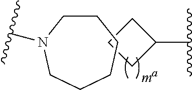

wherein m^{a} is 1, 2, or 3.

In certain embodiments of Formula (III)-(VB),

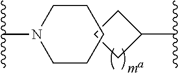

is

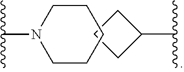

In certain embodiments of Formula (III)-(VB),

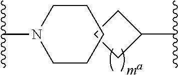

is

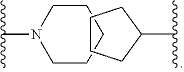

In certain embodiments of Formula (III)-(VB),

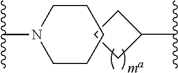

is

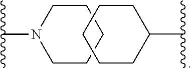

In certain embodiments of Formula (III)-(VB),

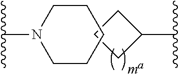

is

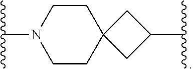 [SMILES: *C1CC2(CCN(*)CC2)C1]

In certain embodiments of Formula (III)-(VB),

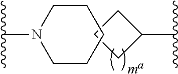

is

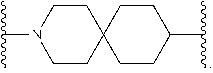 [SMILES: *C1CCC2(CC1)CCN(*)CC2]

In certain embodiments of Formula (III)-(VB), including any of the foregoing, Ring B of L^{1a} is

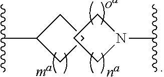

In certain embodiments of Formula (III)-(VB),

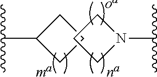

is

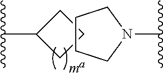

wherein m^{a} is 1, 2, or 3. In certain embodiments of Formula (III)-(VB),

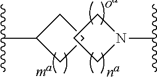

is

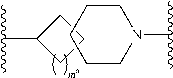

wherein m^{a} is 1, 2, or 3. In certain embodiments of Formula (III)-(VB),

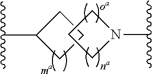

is

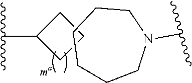

wherein m^{a} is 1, 2, or 3. In certain embodiments of Formula (III)-(VB),

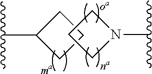

is

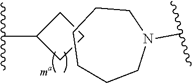

wherein m^{a} is 1, 2, or 3.

In certain embodiments of Formula (III)-(VB),

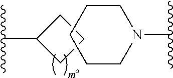

is

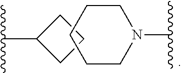

In certain embodiments of Formula (III)-(VB), is

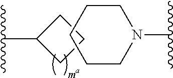

is

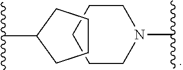

In certain embodiments of Formula (III)-(VB),

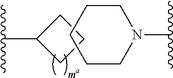

is

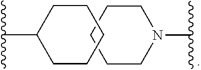

In certain embodiments,

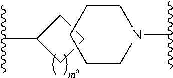

is

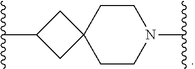 [SMILES: *C1CC2(CCN(*)CC2)C1]

In certain embodiments of Formula (III)-(VB),

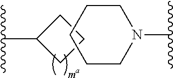

is

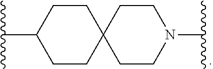 [SMILES: *C1CCC2(CC1)CCN(*)CC2]

In certain embodiments of Formula (III)-(VB), including any of the foregoing, Ring B of L^{1a} is

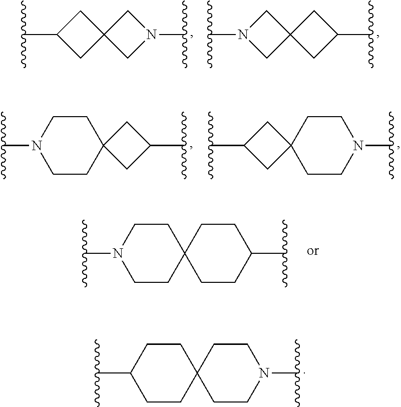

In certain embodiments of Formula (III)-(VB), including any of the foregoing, Ring B of L^{1a} is selected from

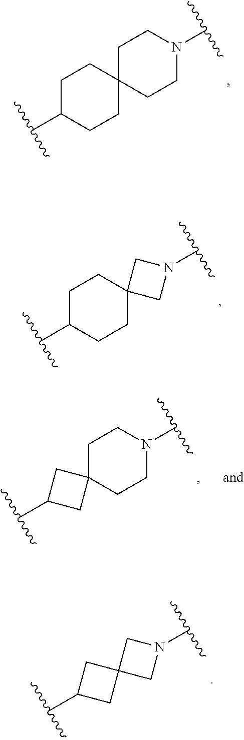 [SMILES: *C1CCC2(CC1)CCN(*)CC2 ; *C1CCC2(CC1)CN(*)C2 ; *C1CC2(CCN(*)CC2)C1 ; *C1CC2(C1)CN(*)C2]

In certain embodiments of Formula (III)-(VB), including any of the foregoing, Ring B of L^{1a} is selected from

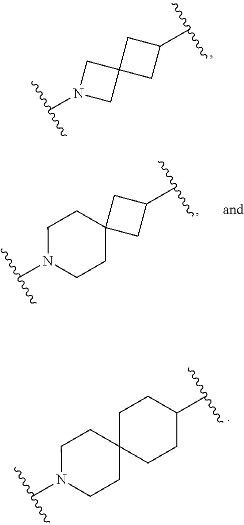 [SMILES: *C1CC2(C1)CN(*)C2 ; *C1CC2(CCN(*)CC2)C1 ; *C1CCC2(CC1)CCN(*)CC2]

In certain embodiments of Formula (III)-(VB), including any of the foregoing, L^{1a} is selected from

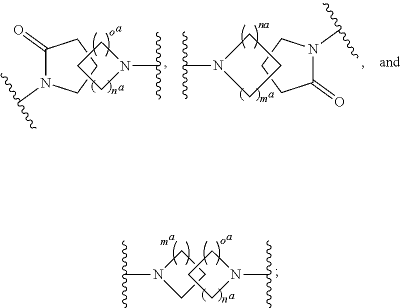

wherein m^{a} is an integer selected from 1, 2, 3, 4, and 5; and
each of n^{a} and o^{a} is an integer independently selected from 1, 2, and 3.

In certain embodiments of Formula (III)-(VB), including any of the foregoing, L^{1a} is selected from

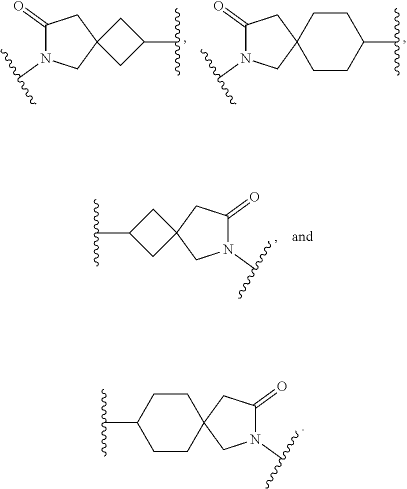 [SMILES: *C1CC2(CC(=O)N(*)C2)C1 ; *C1CCC2(CC1)CC(=O)N(*)C2 ; *C1CC2(CC(=O)N(*)C2)C1 ; *C1CCC2(CC1)CC(=O)N(*)C2]

In certain embodiments of Formula (III)-(VB), including any of the foregoing, Ring B of L^{1a} is selected from

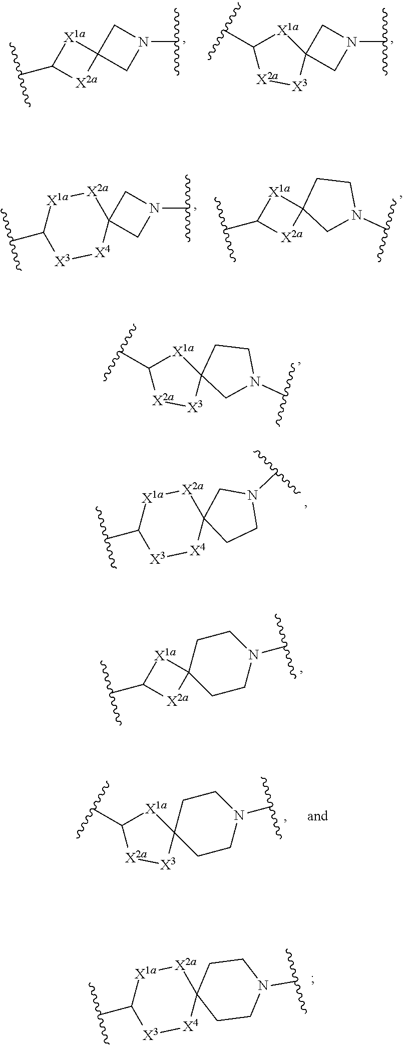

wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}-X³ is —C(R⁴)₂— and when X^{1a}, X^{2a}, X³, and X⁴ are present, at least two of X^{1a}-X⁴ are —C(R⁴)₂—; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group

In certain embodiments of Formula (III)-(VB), including any of the foregoing, Ring B of L^{1a} is selected from

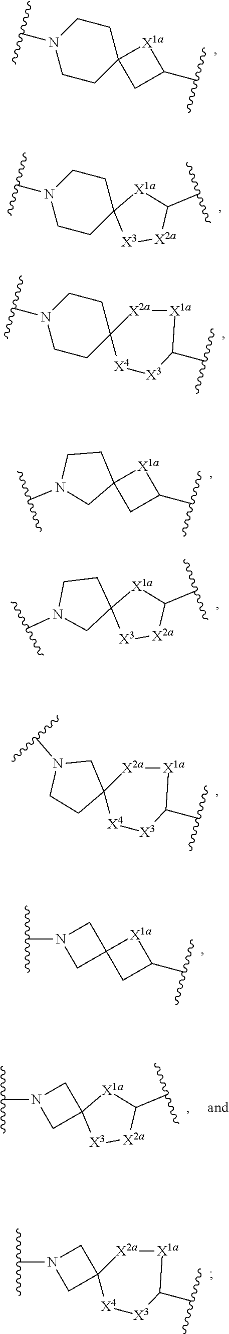

wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}-X³ is —C(R⁴)₂— and when X^{1a}, X^{2a}, X³, and X⁴ are present, at least two of X^{1a}-X⁴ are —C(R⁴)₂—; and

R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

In certain embodiments of Formula (III)-(VB), including any of the foregoing, Ring A of L^{1a} is an optionally substituted bridged, fused, or spirocyclic bicyclic carbocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—, —C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, Ring A of L^{1a} is an optionally substituted C₄₋₁₂ bridged, fused, or spirocyclic bicyclic carbocycle. In certain embodiments of Formula (III)-(VB), including any of the foregoing, Ring A of L^{1a} is an optionally substituted C₄₋₁₂ bridged bicyclic carbocycle. In certain embodiments of Formula (III)-(VB), including any of the foregoing, Ring A of L^{1a} is an optionally substituted C₄₋₈ bridged bicyclic carbocycle.

In certain embodiments of Formula (III)-(VB), including any of the foregoing, Ring A of L^{1a} is selected from

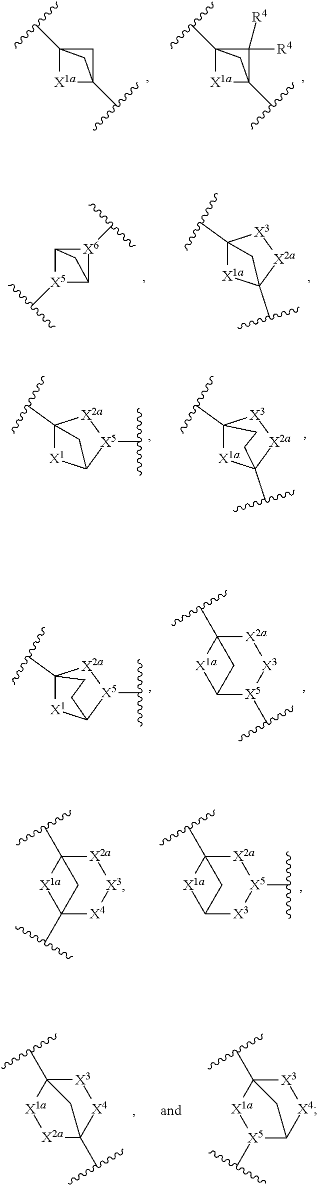

wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}-X³ is —C(R⁴)₂— and when X^{1a}, X^{2a}, X³, and X⁴ are present, at least two of X^{1a}-X⁴ are —C(R⁴)₂—;
X⁵ is CR⁴ or N; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

In certain embodiments of Formula (III)-(VB), including any of the foregoing, Ring A of L^{1a} is selected from

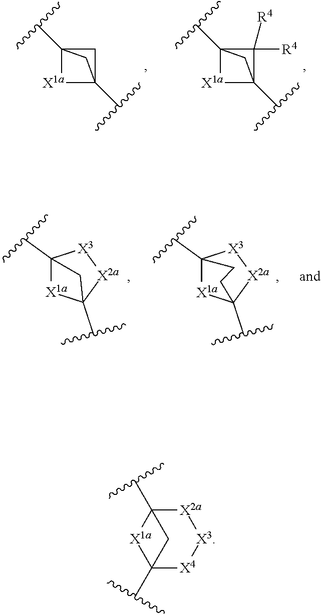

In certain embodiments of Formula (III)-(VB), including any of the foregoing, Ring A of L^{1a} is selected from

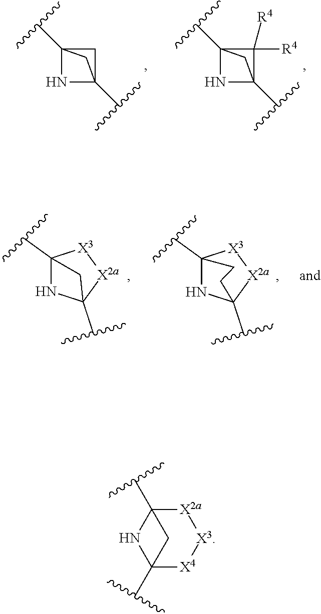

In certain embodiments of Formula (III)-(VB), including any of the foregoing, Ring A of L^{1a} is selected from

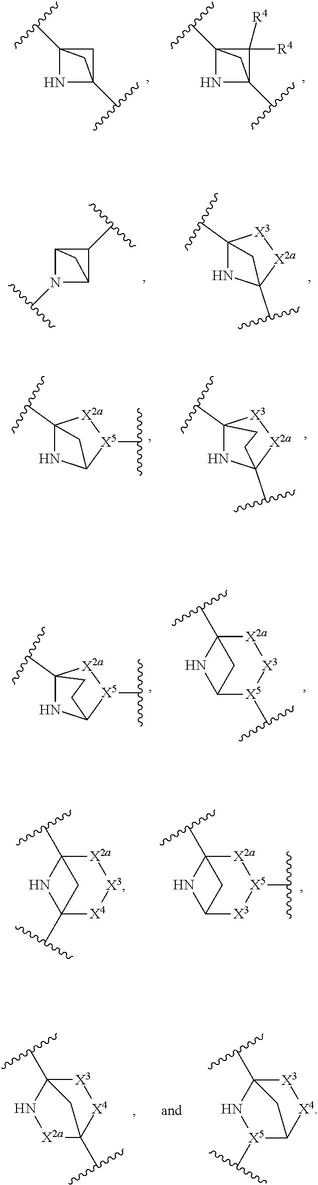

In certain embodiments of Formula (III)-(VB), including any of the foregoing, Ring A of L^{1a} is selected from

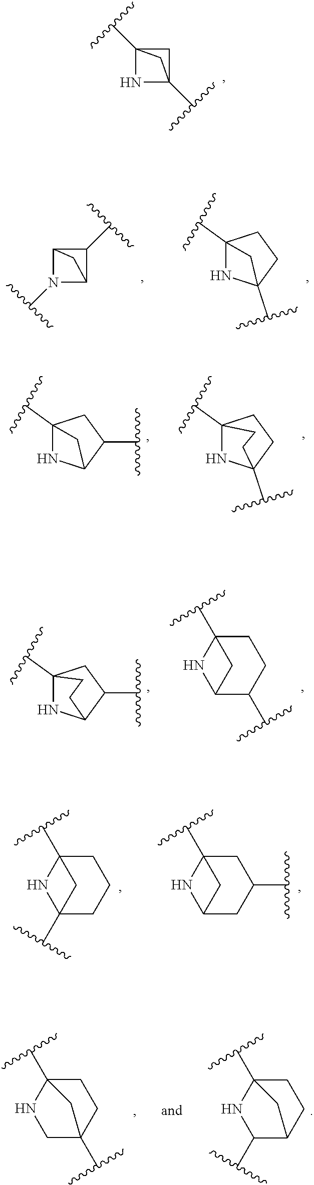

In certain embodiments of Formula (III)-(VB), including any of the foregoing, Ring A of L^{1a} is

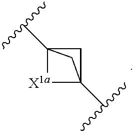

In certain embodiments of Formula (III)-(VB), including any of the foregoing, Ring A of L^{1a} is

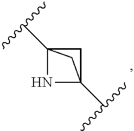 [SMILES: *C12CC(*)(C1)N2]

In certain embodiments of Formula (III)-(VB), including any of the foregoing, X^{1a}, X^{2a}, X³, and/or X⁴ is —C(R⁴)₂—. In certain embodiments of Formula (III)-(VB), including any of the foregoing, X¹a and X²a are —C(R⁴)₂—. In certain embodiments of Formula (III)-(VB), including any of the foregoing, X¹a, X^{2a}, and X³ are —C(R⁴)₂—. In certain embodiments of Formula (III)-(VB), including any of the foregoing, X¹a, X^{2a}, X³, and X⁴ are —C(R⁴)₂—. In certain embodiments of Formula (III)-(VB), including any of the foregoing, X^{1a} is —NH—. In certain embodiments of Formula (III)-(VB), including any of the foregoing, X^{2a} is NH—. In certain embodiments of Formula (III)-(VB), including any of the foregoing, X³ is —NH—. In certain embodiments of Formula (III)-(VB), including any of the foregoing, X⁴ is NH—. In certain embodiments, including any of the foregoing, X^{1a} is —O—. In certain embodiments of Formula (III)-(VB), including any of the foregoing, X^{2a} is —O—. In certain embodiments of Formula (III)-(VB), including any of the foregoing, X³ is —O—. In certain embodiments of Formula (III)-(VB), including any of the foregoing, X⁴ is —O—.

In certain embodiments of Formula (III)-(VB), including any of the foregoing, L^{1a} is

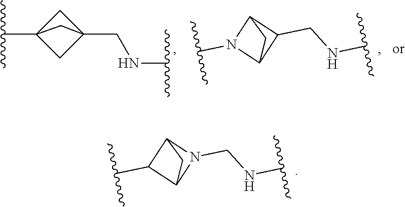

In certain embodiments, L^{1a} is

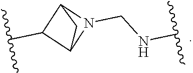 [SMILES: *NCN1C2CC1C2*]

In certain embodiments, including any of the foregoing, a is 0. In certain embodiments, including any of the foregoing, a is 1. In certain embodiments, including any of the foregoing, a is 2. In certain embodiments, including any of the foregoing, a is 3. In certain embodiments, including any of the foregoing, a is 4. In certain embodiments, including any of the foregoing, a is 5. In certain embodiments, including any of the foregoing, a is 6.

In certain embodiments of Formula (III)-(VB), b is 0. In certain embodiments, b is 1.

In certain embodiments of Formula (III)-(VB), b is 0 and a is 0. In certain embodiments of Formula (III)-(VB), b is 0 and a is 1. In certain embodiments of Formula (III)-(VB), b is 0 and a is 2. In certain embodiments of Formula (III)-(VB), b is 0 and a is 3. In certain embodiments of Formula (III)-(VB), b is 0 and a is 4. In certain embodiments of Formula (III)-(VB), b is 0 and a is 5. In certain embodiments of Formula (III)-(VB), b is 0 and a is 6. In certain embodiments of Formula (III)-(VB), b is 1 and a is 1. In certain embodiments of Formula (III)-(VB), b is 1 and a is 2. In certain embodiments of Formula (III)-(VB), b is 1 and a is 3. In certain embodiments of Formula (III)-(VB), b is 1 and a is 4. In certain embodiments of Formula (III)-(VB), b is 1 and a is 5. In certain embodiments of Formula (III)-(VB), b is 1 and a is 6.

In certain embodiments of Formula (III)-(VB), including any of the foregoing, R¹ is hydrogen. In certain embodiments of Formula (III)-(VB), including any of the foregoing, R¹ is unsubstituted alkyl. In certain embodiments of Formula (III)-(VB), including any of the foregoing, R¹ is methyl. In certain embodiments of Formula (III)-(VB), including any of the foregoing, R¹ is alkyl optionally substituted with one or more substituent selected from cycloalkyl, halogen, alkoxy, —CN, —NO₂, and —OH.

In certain embodiments of Formula (III)-(IVB), L⁵ is a linker that comprises at least one amino acid selected from sulfoalanine, hydroxyproline (Hyp), beta-alanine, citrulline (Cit), ornithine (Orn), norleucine (Nle), 3-nitrotyrosine, nitroarginine, pyroglutamic acid (Pyr), naphtylalanine (Nal), 2,4-diaminobutyric acid (DAB), methionine sulfoxide, and methionine sulfone. In certain embodiments of Formula (III)-(IVB), L⁵ is a linker that comprises

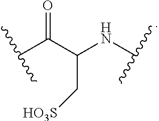 [SMILES: *NC(CS(=O)(=O)O)C(*)=O]

In certain embodiments of Formula (III)-(IVB), L⁵ is a linker that comprises

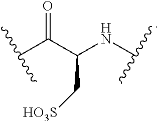 [SMILES: *N[C@@H](CS(=O)(=O)O)C(*)=O]

In certain embodiments of Formula (III)-(IVB), L⁵ is

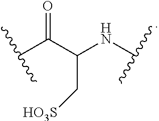 [SMILES: *NC(CS(=O)(=O)O)C(*)=O]

In certain embodiments of Formula (III)-(IVB), L⁵ is

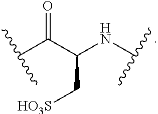 [SMILES: *N[C@@H](CS(=O)(=O)O)C(*)=O]

In certain embodiments, including any of the foregoing, R^{a} is hydrogen and R^{b} is selected from hydrogen, alkyl, alkenyl, alkynyl, cycloalkyl, halogen, alkoxy, —CN, —NO₂, —OH, —N(R²R³)₂,
—C(O)N(R²R³)₂, —C(O)OR², aminoalkyl, hydroxyalkyl, haloalkyl, aryl, arylalkyl, heteroaryl, and heteroarylalkyl. In certain embodiments, including any of the foregoing, R^{a} is hydrogen and R^{b} is selected from hydrogen, alkyl, alkenyl, alkynyl, cycloalkyl, halogen, alkoxy, —CN, —NO₂,
—OH, —NH₂, —C(O)NH₂, —C(O)OH, aminoalkyl, hydroxyalkyl, haloalkyl, aryl, arylalkyl, heteroaryl, and heteroarylalkyl. In certain embodiments, including any of the foregoing, R^{a} is hydrogen and R^{b} is selected from hydrogen, alkyl, halogen, alkoxy, —CN, —NO₂, —OH, —NH₂, —C(O)NH₂, and —C(O)OH. In certain embodiments, including any of the foregoing, R^{a} and R^{b} are both hydrogen.

In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 1; and b is 1. In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 2; and b is 1. In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is hydrogen; a is 3; and b is 1.

In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 1; and b is 0. In certain embodiments of Formula (III)-(VB), including any of the foregoing, R^{a} is hydrogen; R^{b} is selected from hydrogen, alkyl, halogen, alkoxy, —CN,
—NO₂, —OH, —NH₂, —C(O)NH₂, and —C(O)OH; a is 2, and b is 0. In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 3, and b is 0.

In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 1; and b is 1. In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 2; and b is 1. In certain embodiments of Formula (III)-(VB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; R¹ is methyl; a is 3, and b is 1.

In certain embodiments, including any of the foregoing of Formula (III)-(VB), R^{a} and R^{b} are both hydrogen; R¹ is hydrogen; a is 1, and b is 1. In certain embodiments of Formula (III)-(VB), including any of the foregoing, R^{a} and R^{b} are both hydrogen; R¹ is hydrogen; a is 2, and b is 1. In certain embodiments of Formula (III)-(VB), including any of the foregoing, R^{a} and R^{b} are both hydrogen; R¹ is hydrogen; a is 3, and b is 1. In certain embodiments of Formula (III)-(VB), including any of the foregoing, R^{a} and R^{b} are both hydrogen; R¹ is hydrogen; a is 4, and b is 1. In certain embodiments of Formula (III)-(VB), including any of the foregoing, R^{a} and R^{b} are both hydrogen; R¹ is hydrogen; a is 5, and b is 1. In certain embodiments of Formula (III)-(VB), including any of the foregoing, R^{a} and R^{b} are both hydrogen; R¹ is hydrogen; a is 6, and b is 1.

In certain embodiments of Formula (III)-(VB), including any of the foregoing, R^{a} and R^{b} are both hydrogen; a is 1; and b is 0. In certain embodiments of Formula (III)-(VB), including any of the foregoing, R^{a} and R^{b} are both hydrogen; a is 2; and b is 0. In certain embodiments of Formula (III)-(VB), including any of the foregoing, R^{a} and R^{b} are both hydrogen; a is 3; and b is 0. In certain embodiments of Formula (III)-(VB), including any of the foregoing, R^{a} and R^{b} are both hydrogen; a is 4; and b is 0. In certain embodiments of Formula (III)-(VB), including any of the foregoing, R^{a} and R^{b} are both hydrogen; a is 5; and b is 0. In certain embodiments of Formula (III)-(VB), including any of the foregoing, R^{a} and R^{b} are both hydrogen; a is 6; and b is 0.

In certain embodiments of Formula (III)-(VB), including any of the foregoing, R^{a} and R^{b} are both hydrogen; R¹ is methyl; a is 1; and b is 0. In certain embodiments of Formula (III)-(VB), including any of the foregoing, R^{a} and R^{b} are both hydrogen; R¹ is methyl; a is 2; and b is 0. In certain embodiments of Formula (III)-(VB), including any of the foregoing, R^{a} and R^{b} are both hydrogen; R¹ is methyl; a is 3; and b is 0. In certain embodiments of Formula (III)-(VB), including any of the foregoing, R^{a} and R^{b} are both hydrogen; R¹ is methyl; a is 4; and b is 0. In certain embodiments of Formula (III)-(VB), including any of the foregoing, R^{a} and R^{b} are both hydrogen; R¹ is methyl; a is 5; and b is 0. In certain embodiments of Formula (III)-(VB), including any of the foregoing, R^{a} and R^{b} are both hydrogen; R¹ is methyl; a is 6; and b is 0.

In certain embodiments of Formula (III)-(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 1; and c is 1. In certain embodiments of Formula (III)-(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 2; and c is 1. In certain embodiments of Formula (III)-(IVB), including any of the foregoing, R^{a} is hydrogen; R^{b} is selected from hydrogen, alkyl, halogen, alkoxy, —CN, —NO₂, —OH, —NH₂, —C(O)NH₂, and —C(O)OH; a is 3; and c is 1.

In certain embodiments of Formula (III)-(IVB), including any of the foregoing, Y^{a} is
*—C(O)—(CR^{a}R^{b})_{c}—NH— wherein * represents where Y^{a} is bound to RL. In certain embodiments of Formula (III)-(IVB), including any of the foregoing, Y^{a} is *—C(O)—(CH₂)_{c}—NH—. In certain embodiments of Formula (III)-(IVB), including any of the foregoing, Y^{a} is *—C(O)—(CH₂)—NH—. In certain embodiments, including any of the foregoing, Y^{a} is *—C(O)—(CH₂)₂—NH—. In certain embodiments of Formula (III)-(IVB), including any of the foregoing, Y^{a} is *—C(O)—(CH₂)₃—NH—. In certain embodiments of Formula (III)-(IVB), including any of the foregoing, Y^{a} is *—C(O)—(CH₂)₄—NH—. In certain embodiments of Formula (III)-(IVB), including any of the foregoing, Y^{a} is *—C(O)—(CH₂)₅—NH—. In certain embodiments of Formula (III)-(IVB), including any of the foregoing, Y^{a} is *—C(O)—(CH₂)₆—NH—.

In certain embodiments of Formula (III)-(IVB), including any of the foregoing, Y^{a} is
*—C(O)—(CR^{a}R^{b})—NH—. In certain embodiments of Formula (III)-(IVB), including any of the foregoing, Y^{a} is *—C(O)—(CR^{a}R^{b})₂—NH—. In certain embodiments of Formula (III)-(IVB), including any of the foregoing, Y^{a} is *—C(O)—(CR^{a}R^{b})₃—NH—. In certain embodiments of Formula (III)-(IVB), including any of the foregoing, Y^{a} is *—C(O)—(CR^{a}R^{b})₄—NH—. In certain embodiments of Formula (III)-(IVB), including any of the foregoing, Y^{a} is *—C(O)—(CR^{a}R^{b})₅—NH—. In certain embodiments of Formula (III)-(IVB), including any of the foregoing, Y^{a} is *—C(O)—(CR^{a}R^{b})₆—NH—.

In certain embodiments of Formula (III)-(IVB), including any of the foregoing, Y^{a} is
*—C(O)—(CR^{a}R^{b})_{c}—NH— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments of Formula (III)-(IVB), including any of the foregoing, Y^{a} is *—C(O)—(CR^{a}R^{b})₂—NH—, *—C(O)—(CR^{a}R^{b})₃—NH—, or *—C(O)—(CR^{a}R^{b})₄—NH— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl.

In certain embodiments of Formula (III)-(IVB), including any of the foregoing, Y^{a} is
*—C(O)—(CR^{a}R^{b})_{c}— wherein * represents where Y^{a} is bound to RL. In certain embodiments of Formula (III)-(IVB), including any of the foregoing, Y^{a} is *—C(O)—(CH₂)_{c}—. In certain embodiments of Formula (III)-(IVB), including any of the foregoing, Y^{a} is *—C(O)—(CH₂)—. In certain embodiments of Formula (III)-(IVB), including any of the foregoing, Y^{a} is *—C(O)—(CH₂)₂—. In certain embodiments of Formula (III)-(IVB), including any of the foregoing, Y^{a} is *—C(O)—(CH₂)₃—. In certain embodiments of Formula (III)-(IVB), including any of the foregoing, Y^{a} is *—C(O)—(CH₂)₄—. In certain embodiments of Formula (III)-(IVB), including any of the foregoing of Formula (III)-(IVB), Y^{a} is *—C(O)—(CH₂)₅—. In certain embodiments, including any of the foregoing, Y^{a} is *—C(O)—(CH₂)₆—.

In certain embodiments, including any of the foregoing of Formula (III)-(IVB), Y^{a} is
*—C(O)—(CR^{a}R^{b})—. In certain embodiments of Formula (III)-(IVB), including any of the foregoing, Y^{a} is *—C(O)—(CR^{a}R^{b})₂—. In certain embodiments of Formula (III)-(IVB), including any of the foregoing, Y^{a} is *—C(O)—(CR^{a}R^{b})₃—. In certain embodiments of Formula (III)-(IVB), including any of the foregoing, Y^{a} is *—C(O)—(CR^{a}R^{b})₄—. In certain embodiments of Formula (III)-(IVB), including any of the foregoing, Y^{a} is *—C(O)—(CR^{a}R^{b})₅—. In certain embodiments of Formula (III)-(IVB), including any of the foregoing, Y^{a} is *—C(O)—(CR^{a}R^{b})₆—.

In certain embodiments of Formula (III)-(IVB), including any of the foregoing, Y^{a} is
*—C(O)—(CR^{a}R^{b})_{c}— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl. In certain embodiments of Formula (III)-(IVB), including any of the foregoing, Y^{a} is *—C(O)—(CR^{a}R^{b})₂—, *—C(O)—(CR^{a}R^{b})₃—, or *—C(O)—(CR^{a}R^{b})₄— wherein R^{a} is hydrogen and R^{b} is selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl.

In certain embodiments of Formula (III)-(IVB), including any of the foregoing, Y^{a} is *—C(O)—(CH₂)₂—NH— or *—C(O)—(CH₂)₄—.

In certain embodiments of Formula (III)-(IVB), including any of the foregoing, L² is absent. In certain embodiments of Formula (III)-(IVB), including any of the foregoing, L² is a linker comprising a hydrophilic polymer residue.

In certain embodiments of Formula (III)-(IVB), including any of the foregoing, L² is —(CR^{a}R^{b})ₐ-POLY¹-. In certain embodiments of Formula (III)-(IVB), including any of the foregoing, L² is —CH₂-POLY¹-. In certain embodiments of Formula (III)-(IVB), including any of the foregoing, L² is —(CH₂)₂-POLY¹-. In certain embodiments of Formula (III)-(IVB), including any of the foregoing, L² is —(CH₂)₃-POLY¹-. In certain embodiments of Formula (III)-(IVB), including any of the foregoing, L² is —(CH₂)₄-POLY¹-. In certain embodiments of Formula (III)-(IVB), including any of the foregoing, L² is —(CH₂)₅-POLY¹-. In certain embodiments of Formula (III)-(IVB), including any of the foregoing, L² is —(CH₂)₆-POLY¹-. In certain embodiments of Formula (III)-(IVB), including any of the foregoing, L² is —CR^{a}R^{b}-POLY¹-. In certain embodiments of Formula (III)-(IVB), including any of the foregoing, L² is —(CR^{a}R^{b})₂-POLY¹-. In certain embodiments, including any of the foregoing, L² is —(CR^{a}R^{b})₃-POLY¹-. In certain embodiments of Formula (III)-(IVB), including any of the foregoing, L² is —(CR^{a}R^{b})₄-POLY¹-.

In certain embodiments of Formula (III)-(IVB), including any of the foregoing, L² is —POLY¹-.

In certain embodiments of Formula (III)-(IVB), including any of the foregoing, L² is —(CR^{a}R^{b})ₐ-POLY¹-(CR^{a}R^{b})ₐ—. In certain embodiments of Formula (III)-(IVB), including any of the foregoing, L² is —(CR^{a}R^{b})ₐ-POLY¹-(CR^{a}R^{b})ₐ— wherein a is independently selected from 0, 1, 2, 3, 4, 5, or 6. In certain embodiments of Formula (III)-(IVB), including any of the foregoing, L² is —(CH₂)ₐ-POLY¹-(CH₂)ₐ— wherein a is independently selected from 0, 1, 2, 3, 4, 5, or 6. In certain embodiments of Formula (III)-(IVB), including any of the foregoing, L² is
—(CH₂)ₐ-POLY¹-(CR^{a}R^{b})ₐ— wherein a is selected from 1, 2, 3, 4, 5, or 6. In certain embodiments of Formula (III)-(IVB), including any of the foregoing, L² is —(CR^{a}R^{b})ₐ-POLY¹-(CH₂)ₐ— wherein a is independently selected from 0, 1, 2, 3, 4, 5, or 6.

In certain embodiments of Formula (III)-(IVB), including any of the foregoing, POLY¹ is a diavalent residue of a nonpeptidic, hydrophilic polymer. In certain embodiments of Formula (III)-(IVB), POLY¹ is a diavalent residue of polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(□-hydroxy acid), poly(vinyl alcohol), polyphosphazene, polyoxazolines (POZ), poly(N-acryloylmorpholine), polysarcosine, or a combination thereof. In certain embodiments of Formula (III)-(IVB), including any of the foregoing, POLY¹ is a divalent residue of polyethylene glycol (PEG), poly(propylene glycol) (PPG), or a copolymer of ethylene glycol and propylene glycol.

In certain embodiments of Formula (III)-(IVB), including any of the foregoing, POLY¹ is a diavalent residue of polyethylene glycol (PEG). In certain embodiments of Formula (III)-(IVB), including any of the foregoing, POLY¹ is a diavalent residue of poly(propylene glycol) (PPG). In certain embodiments of Formula (III)-(IVB), including any of the foregoing, POLY¹ is a diavalent residue of copolymers of ethylene glycol and propylene glycol. In certain embodiments of Formula (III)-(IVB), including any of the foregoing, POLY¹ is a diavalent residue of poly(oxyethylated polyol). In certain embodiments of Formula (III)-(IVB), including any of the foregoing, POLY¹ is a diavalent residue of poly(olefinic alcohol). In certain embodiments of Formula (III)-(IVB), including any of the foregoing, POLY¹ is a diavalent residue of poly(vinylpyrrolidone). In certain embodiments of Formula (III)-(IVB), including any of the foregoing, POLY¹ is a diavalent residue of poly(hydroxyalkylmethacrylamide). In certain embodiments of Formula (III)-(IVB), including any of the foregoing, POLY¹ is a diavalent residue of poly(hydroxyalkylmethacrylate). In certain embodiments of Formula (III)-(IVB), including any of the foregoing, POLY¹ is a diavalent residue of poly(saccharides). In certain embodiments of Formula (III)-(IVB), including any of the foregoing, POLY¹ is a diavalent residue of poly(□-hydroxy acid). In certain embodiments of Formula (III)-(IVB), including any of the foregoing, POLY¹ is a diavalent residue of poly(vinyl alcohol). In certain embodiments of Formula (III)-(IVB), including any of the foregoing, POLY¹ is a diavalent residue of polyphosphazene. In certain embodiments of Formula (III)-(IVB), including any of the foregoing, POLY¹ is a diavalent residue of polyoxazolines (POZ). In certain embodiments of Formula (III)-(IVB), including any of the foregoing, POLY¹ is a diavalent residue of poly(N-acryloylmorpholine). In certain embodiments of Formula (III)-(IVB), including any of the foregoing, POLY¹ is a diavalent residue of polysarcosine.

In certain embodiments of Formula (III)-(IVB), including any of the foregoing, POLY¹ is

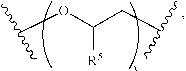

wherein R⁵ is hydrogen or methyl, x is an integer from 1 to 100, inclusive, and

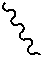

represents attachment to the remainder of the compound or conjugate. In certain embodiments of Formula (III)-(IVB), including any of the foregoing, x is an integer between 1 to 25. In certain embodiments of Formula (III)-(IVB), including any of the foregoing, x is an integer between 5 to 15. In some embodiments of Formula (III)-(IVB), including any of the foregoing, x is 1. In some embodiments of Formula (III)-(IVB), including any of the foregoing, x is 2. In some embodiments of Formula (III)-(IVB), including any of the foregoing, x is 3. In some embodiments of Formula (III)-(IVB), including any of the foregoing, x is 4. In some embodiments of Formula (III)-(IVB), including any of the foregoing, x is 5. In some embodiments of Formula (III)-(IVB), including any of the foregoing, x is 6. In some embodiments of Formula (III)-(IVB), including any of the foregoing, x is 7. In some embodiments of Formula (III)-(IVB), including any of the foregoing, x is 8. In some embodiments of Formula (III)-(IVB), including any of the foregoing, x is 9. In some embodiments of Formula (III)-(IVB), including any of the foregoing, x is 10. In some embodiments of Formula (III)-(IVB), including any of the foregoing, x is 11. In some embodiments of Formula (III)-(IVB), including any of the foregoing, x is 12. In some embodiments of Formula (III)-(IVB), including any of the foregoing, x is 13. In some embodiments of Formula (III)-(IVB), including any of the foregoing, x is 14. In some embodiments of Formula (III)-(IVB), including any of the foregoing, x is 15. In some embodiments of Formula (III)-(IVB), including any of the foregoing, x is 16. In some embodiments of Formula (III)-(IVB), including any of the foregoing, x is 17. In some embodiments, including any of the foregoing, x is 18. In some embodiments of Formula (III)-(IVB), including any of the foregoing, x is 19. In some embodiments of Formula (III)-(IVB), including any of the foregoing, x is 20. In certain embodiments of Formula (III)-(IVB), including any of the foregoing, x is an integer between 25 and 50. In certain embodiments of Formula (III)-(IVB), including any of the foregoing, x is an integer between 35 and 45. In certain embodiments of Formula (III)-(IVB), including any of the foregoing, x is an integer between 50 and 75. In certain embodiments of Formula (III)-(IVB), including any of the foregoing, x is an integer between 55 and 65. In certain embodiments of Formula (III)-(IVB), including any of the foregoing, x is an integer between 75 and 100. In certain embodiments of Formula (III)-(IVB), including any of the foregoing, x is an integer between 85 and 95. In certain embodiments of Formula (III)-(IVB), including any of the foregoing, x is an integer in the range of 1 and 25, 20 and 45, 40 and 65, 60 and 85, 70 and 95, or 75 and 100.

In some embodiments of Formula (III)-(IVB), including any of the foregoing, R⁵ is hydrogen. In some embodiments, including any of the foregoing, R⁵ is methyl.

In certain embodiments of Formula (III)-(IVB), including any of the foregoing, L² is
—(CR^{a}R^{b})ₐ-POLY¹- wherein POLY¹ is

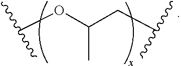

In certain embodiments of Formula (III)-(IVB), including any of the foregoing, L² is —(CR^{a}R^{b})ₐ-POLY¹- wherein POLY¹ is

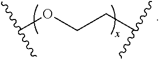

In certain embodiments of Formula (III)-(IVB), including any of the foregoing, L² is —(CH₂)ₐ-POLY¹- wherein POLY¹ is

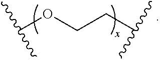

In certain embodiments of Formula (III)-(IVB), including any of the foregoing, L² is —(CH₂)₂-POLY¹- wherein POLY¹ is

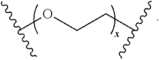

In certain embodiments of Formula (III)-(IVB), including any of the foregoing, L² is —(CR^{a}R^{b})ₐ-POLY¹- wherein POLY¹ is

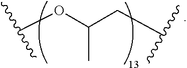

In certain embodiments of Formula (III)-(IVB), including any of the foregoing, L² is —(CR^{a}R^{b})ₐ-POLY¹- wherein POLY¹ is

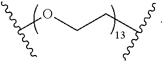

In certain embodiments of Formula (III)-(IVB), including any of the foregoing, L² is —(CH₂)₂-POLY¹- wherein POLY¹ is

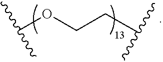

In certain embodiments of Formula (III)-(IVB), including any of the foregoing, L² is —(CH₂)₂-POLY¹- wherein POLY¹ is

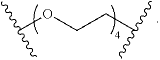

In certain embodiments of Formula (III)-(IVB), including any of the foregoing, L² is —(CH₂)₂-POLY¹- wherein POLY¹ is

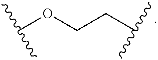 [SMILES: *CCO*]

In certain embodiments of Formula (III)-(IVB), including any of the foregoing, L² is

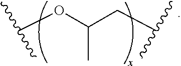

In certain embodiments, including any of the foregoing, L² is

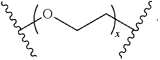

In certain embodiments of Formula (III)-(IVB), including any of the foregoing, L² is

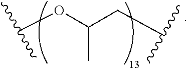

In certain embodiments of Formula (III)-(IVB), including any of the foregoing, L² is

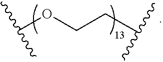

In certain embodiments of Formula (III)-(IVB), including any of the foregoing, L² is —(CR^{a}R^{b})ₐ-POLY¹-(CR^{a}R^{b})ₐ- wherein POLY¹ is

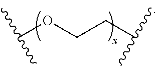

In certain embodiments of Formula (III)-(IVB), including any of the foregoing, L² is —(CR^{a}R^{b})ₐ-POLY¹-(CR^{a}R^{b})ₐ- wherein POLY¹ is

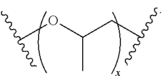

In certain embodiments of Formula (III)-(IVB), including any of the foregoing, L² is —(CR^{a}R^{b})ₐ-POLY¹-(CR^{a}R^{b})ₐ- wherein POLY¹ is

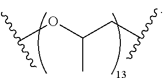

In certain embodiments of Formula (III)-(IVB), including any of the foregoing, L² is —(CR^{a}R^{b})ₐ-POLY¹-(CR^{a}R^{b})ₐ- wherein POLY¹ is

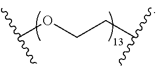

In certain embodiments of Formula (III)-(IVB), including any of the foregoing, L² is selected from

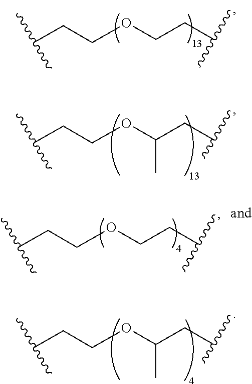

In certain embodiments of Formula (III)-(IVB), including any of the foregoing, L² is

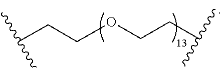

In certain embodiments of Formula (III)-(IVB), including any of the foregoing, L² is

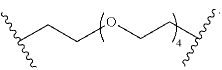

In certain embodiments of Formula (III)-(IVB), including any of the foregoing, L² is

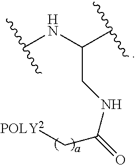

In certain embodiments of Formula (III)-(IVB), including any of the foregoing, L² is

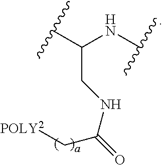

In certain embodiments of Formula (III)-(IVB), including any of the foregoing, L² is

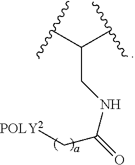

In certain embodiments of Formula (III)-(IVB), including any of the foregoing, L² is

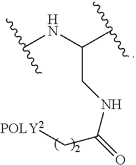

In certain embodiments of Formula (III)-(IVB), including any of the foregoing, L² is

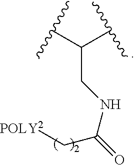

In certain embodiments of Formula (III)-(IVB), including any of the foregoing, POLY² is a residue of a nonpeptidic, hydrophilic polymer. In certain embodiments of Formula (III)-(IVB), POLY² is a residue of polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(□-hydroxy acid), poly(vinyl alcohol), polyphosphazene, polyoxazolines (POZ), poly(N-acryloylmorpholine), polysarcosine, or a combination thereof. In certain embodiments of Formula (III)-(IVB), including any of the foregoing, POLY² is a residue of polyethylene glycol (PEG), methoxypolyethylene glycol (mPEG), poly(propylene glycol) (PPG), or a copolymer of ethylene glycol and propylene glycol. In certain embodiments of Formula (III)-(IVB), including any of the foregoing, POLY² is a residue of methoxypolyethylene glycol (mPEG).

In certain embodiments of Formula (III)-(IVB), including any of the foregoing, POLY² is a residue of polyethylene glycol (PEG). In certain embodiments of Formula (III)-(IVB), including any of the foregoing, POLY² is a residue of poly(propylene glycol) (PPG). In certain embodiments, including any of the foregoing, POLY² is a residue of copolymers of ethylene glycol and propylene glycol. In certain embodiments of Formula (III)-(IVB), including any of the foregoing, POLY² is a residue of poly(oxyethylated polyol). In certain embodiments of Formula (III)-(IVB), including any of the foregoing, POLY² is a residue of poly(olefinic alcohol). In certain embodiments of Formula (III)-(IVB), including any of the foregoing, POLY² is a residue of poly(vinylpyrrolidone). In certain embodiments of Formula (III)-(IVB), including any of the foregoing, POLY² is a residue of poly(hydroxyalkylmethacrylamide). In certain embodiments of Formula (III)-(IVB), including any of the foregoing, POLY² is a residue of poly(hydroxyalkylmethacrylate). In certain embodiments of Formula (III)-(IVB), including any of the foregoing, POLY² is a residue of poly(saccharides). In certain embodiments of Formula (III)-(IVB), including any of the foregoing, POLY² is a residue of poly(□-hydroxy acid). In certain embodiments of Formula (III)-(IVB), including any of the foregoing, POLY² is a residue of poly(vinyl alcohol). In certain embodiments of Formula (III)-(IVB), including any of the foregoing, POLY² is a residue of polyphosphazene. In certain embodiments of Formula (III)-(IVB), including any of the foregoing, POLY² is a residue of polyoxazolines (POZ). In certain embodiments of Formula (III)-(IVB), including any of the foregoing, POLY² is a residue of poly(N-acryloylmorpholine). In certain embodiments of Formula (III)-(IVB), including any of the foregoing, POLY² is a residue of polysarcosine.

In certain embodiments of Formula (III)-(IVB), including any of the foregoing, POLY² is

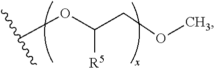

wherein R⁵ is hydrogen or methyl, x is an integer from 1 to 100, inclusive, and

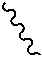

represents attachment to the remainder of the compound or conjugate. In certain embodiments of Formula (III)-(IVB), including any of the foregoing, x is an integer between 1 to 25. In certain embodiments of Formula (III)-(IVB), including any of the foregoing, x is an integer between 5 to 15. In some embodiments of Formula (III)-(IVB), including any of the foregoing, x is 1. In some embodiments of Formula (III)-(IVB), including any of the foregoing, x is 2. In some embodiments of Formula (III)-(IVB), including any of the foregoing, x is 3. In some embodiments of Formula (III)-(IVB), including any of the foregoing, x is 4. In some embodiments of Formula (III)-(IVB), including any of the foregoing, x is 5. In some embodiments of Formula (III)-(IVB), including any of the foregoing, x is 6. In some embodiments, including any of the foregoing, x is 7. In some embodiments of Formula (III)-(IVB), including any of the foregoing, x is 8. In some embodiments of Formula (III)-(IVB), including any of the foregoing, x is 9. In some embodiments of Formula (III)-(IVB), including any of the foregoing, x is 10. In some embodiments of Formula (III)-(IVB), including any of the foregoing, x is 11. In some embodiments of Formula (III)-(IVB), including any of the foregoing, x is 12. In some embodiments of Formula (III)-(IVB), including any of the foregoing, x is 13. In some embodiments of Formula (III)-(IVB), including any of the foregoing, x is 14. In some embodiments of Formula (III)-(IVB), including any of the foregoing, x is 15. In some embodiments of Formula (III)-(IVB), including any of the foregoing, x is 16. In some embodiments of Formula (III)-(IVB), including any of the foregoing of Formula (III)-(IVB), x is 17. In some embodiments, including any of the foregoing of Formula (III)-(IVB), x is 18. In some embodiments of Formula (III)-(IVB), including any of the foregoing, x is 19. In some embodiments of Formula (III)-(IVB), including any of the foregoing, x is 20. In certain embodiments of Formula (III)-(IVB), including any of the foregoing, x is an integer between 25 and 50. In certain embodiments of Formula (III)-(IVB), including any of the foregoing, x is an integer between 35 and 45. In certain embodiments of Formula (III)-(IVB), including any of the foregoing, x is an integer between 50 and 75. In certain embodiments of Formula (III)-(IVB), including any of the foregoing, x is an integer between 55 and 65. In certain embodiments of Formula (III)-(IVB), including any of the foregoing, x is an integer between 75 and 100. In certain embodiments of Formula (III)-(IVB), including any of the foregoing, x is an integer between 85 and 95. In certain embodiments of Formula (III)-(IVB), including any of the foregoing, x is an integer in the range of 1 and 25, 20 and 45, 40 and 65, 60 and 85, 70 and 95, or 75 and 100.

In some embodiments of Formula (III)-(IVB), including any of the foregoing, R⁵ is hydrogen. In some embodiments of Formula (III)-(IVB), including any of the foregoing, R⁵ is methyl.

In certain embodiments of Formula (III)-(IVB), including any of the foregoing, L² is selected from the group consisting of

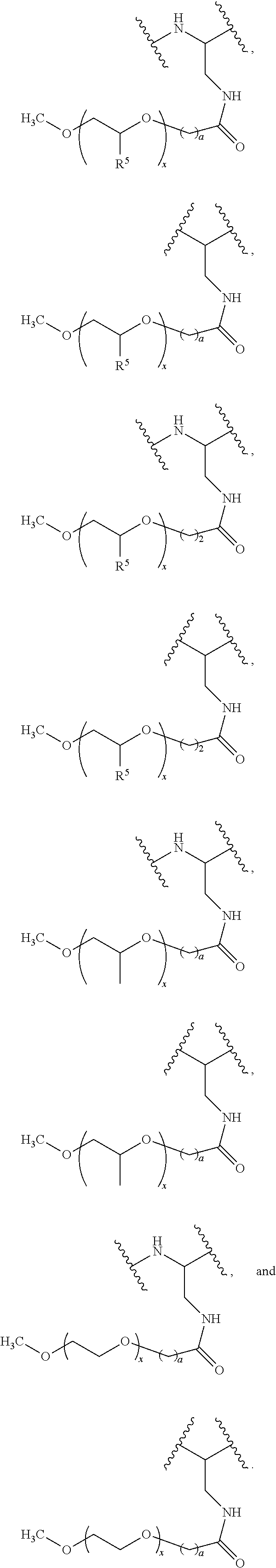

In certain embodiments of Formula (III)-(IVB), including any of the foregoing, L² is selected from the group consisting of

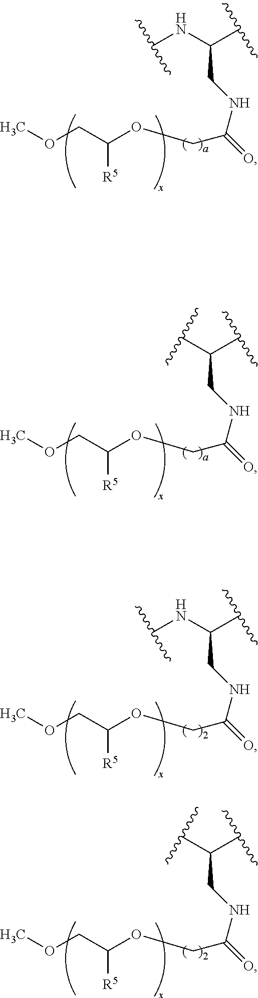

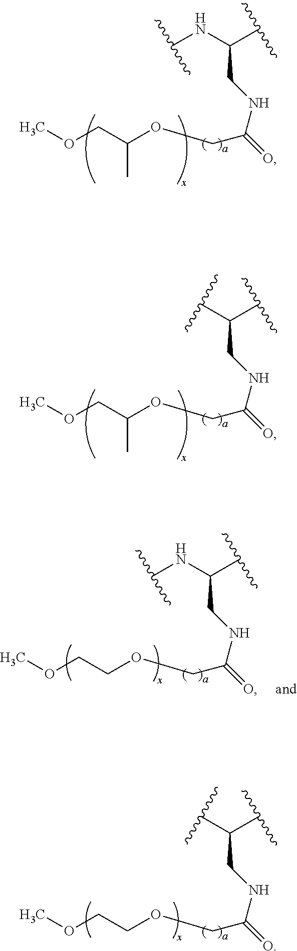

In certain embodiments of Formula (III)-(IVB), including any of the foregoing, L² is

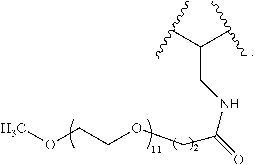

In certain embodiments of Formula (III)-(IVB), including any of the foregoing, L² is

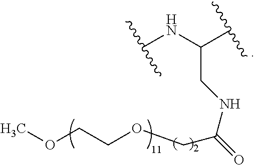

In certain embodiments of Formula (III)-(IVB), including any of the foregoing, L² is

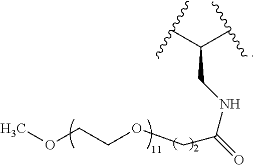

In certain embodiments of Formula (III)-(IVB), including any of the foregoing, L² is

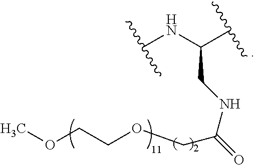

In certain embodiments of Formula (III)-(IVB), including any of the foregoing, L³ is
—C(O)-AA-.

In certain embodiments of Formula (III)-(IVB), including any of the foregoing, L³ is —C(O)-AA-Z—(CR^{a}R^{b})ₐ-Z—(CR^{a}R^{b}) a —C(O)—. In certain embodiments of Formula (III)-(IVB), including any of the foregoing, L³ is —C(O)-AA-NR²—(CR^{a}R^{b})ₐ—NR²—(CR^{a}R^{b})ₐ—C(O)—. In certain embodiments of Formula (III)-(IVB), including any of the foregoing, L³ is —C(O)-AA-NH—(CR^{a}R^{b})ₐ—NH—(CR^{a}R^{b})ₐ—C(O)—. In certain embodiments of Formula (III)-(IVB), including any of the foregoing, L³ is —C(O)-AA-NH—(CH₂)ₐ—NH—(CH₂)ₐ—C(O)—. In certain embodiments of Formula (III)-(IVB), including any of the foregoing, L³ is —C(O)-AA-NH—(CH₂)ₐ—NH—(CH₂)ₐ—C(O)— wherein a is selected from 1, 2, and 3. In certain embodiments, including any of the foregoing, L³ is —C(O)-AA-NH—CH₂—NH—CH₂-C(O)—

In certain embodiments of Formula (III)-(IVB), including any of the foregoing, L³ is —C(O)-AA-Z—(CR^{a}R^{b})ₐ. In certain embodiments, including any of the foregoing, L³ is —C(O)-AA-NR²—(CH₂)ₐ. In certain embodiments of Formula (III)-(IVB), including any of the foregoing, L³ is —C(O)-AA-NH—(CH₂)₂.

In certain embodiments of Formula (III)-(IVB), including any of the foregoing, L³ is -AA-. In certain embodiments, including any of the foregoing, L³ is

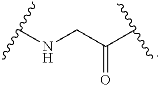 [SMILES: *NCC(*)=O]

In certain embodiments of Formula (III)-(IVB), including any of the foregoing, -AA- is an amino acid residue. In certain embodiments of Formula (III)-(IVB), including any of the foregoing, -AA- is a peptide residue. In certain embodiments of Formula (III)-(IVB), including any of the foregoing, -AA- is a dipeptide residue, a tripeptide residue, a tetrapeptide residue, or a pentapeptide residue. In certain embodiments of Formula (III)-(IVB), including any of the foregoing, -AA- comprises at least one amino acid residue selected from alanine, glycine, valine, and asparagine. In certain embodiments of Formula (III)-(IVB), including any of the foregoing, -AA- comprises at least one amino acid residue selected from alanine and glycine. In certain embodiments of Formula (III)-(IVB), including any of the foregoing, -AA- is selected from the group consisting of

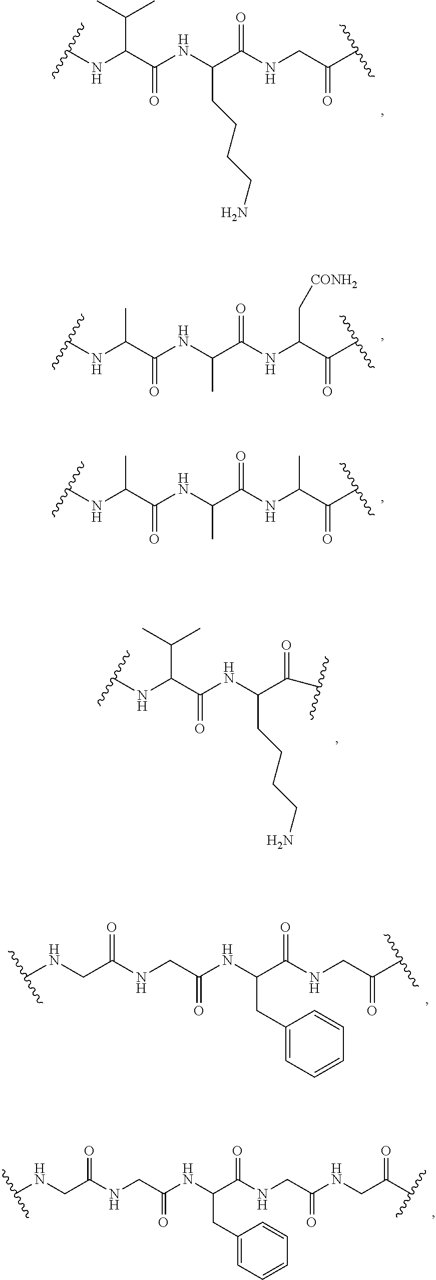 [SMILES: *NC(C(=O)NC(CCCCN)C(=O)NCC(*)=O)C(C)C ; *NC(C)C(=O)NC(C)C(=O)NC(CC(N)=O)C(*)=O ; *NC(C)C(=O)NC(C)C(=O)NC(C)C(*)=O ; *NC(C(=O)NC(CCCCN)C(*)=O)C(C)C ; *NCC(=O)NCC(=O)NC(Cc1ccccc1)C(=O)NCC(*)=O ; *NCC(=O)NCC(=O)NC(Cc1ccccc1)C(=O)NCC(=O)NCC(*)=O]

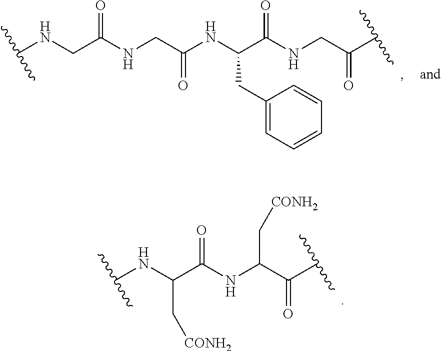 [SMILES: *NCC(=O)NCC(=O)N[C@@H](Cc1ccccc1)C(=O)NCC(*)=O ; *NC(CC(N)=O)C(=O)NC(CC(N)=O)C(*)=O]

In certain embodiments of Formula (III)-(IVB), including any of the foregoing, -AA- is selected from the group consisting of

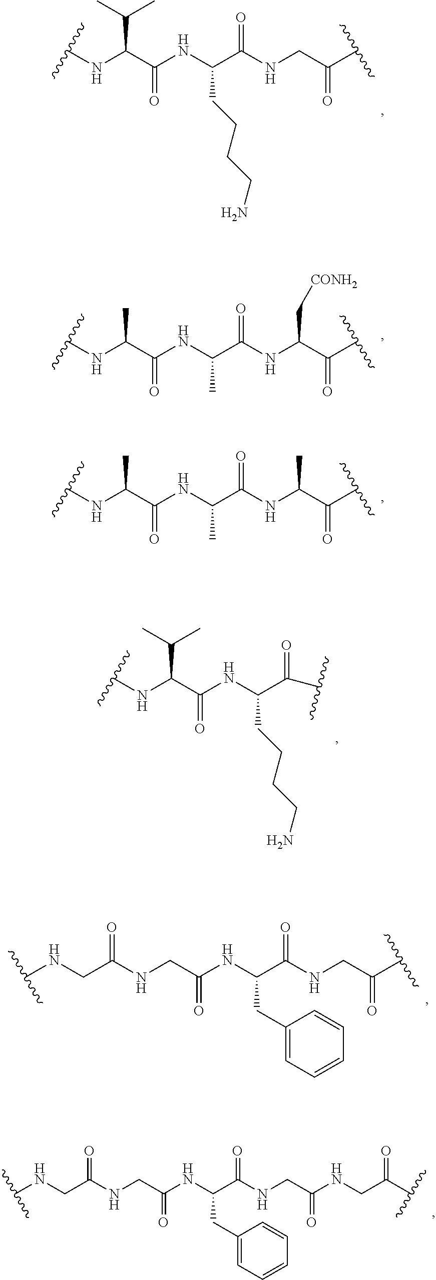 [SMILES: *N[C@H](C(=O)N[C@@H](CCCCN)C(=O)NCC(*)=O)C(C)C ; *N[C@@H](C)C(=O)N[C@@H](C)C(=O)N[C@@H](CC(N)=O)C(*)=O ; *N[C@@H](C)C(=O)N[C@@H](C)C(=O)N[C@@H](C)C(*)=O ; *N[C@H](C(=O)N[C@@H](CCCCN)C(*)=O)C(C)C ; *NCC(=O)NCC(=O)N[C@@H](Cc1ccccc1)C(=O)NCC(*)=O ; *NCC(=O)NCC(=O)N[C@@H](Cc1ccccc1)C(=O)NCC(=O)NCC(*)=O]

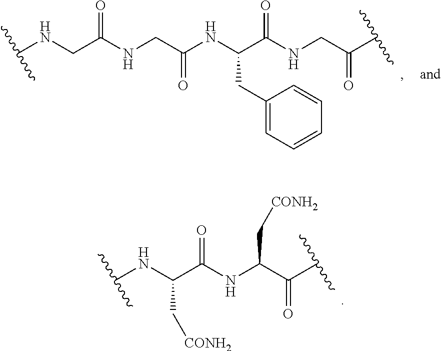 [SMILES: *NCC(=O)NCC(=O)N[C@@H](Cc1ccccc1)C(=O)NCC(*)=O ; *N[C@@H](CC(N)=O)C(=O)N[C@@H](CC(N)=O)C(*)=O]

In certain embodiments of Formula (III)-(IVB), including any of the foregoing, L³ is —C(O)—.

In certain embodiments of Formula (III)-(IVB), including any of the foregoing, L³ is —C(O)—Z—(CR^{a}R^{b})ₐ—C(O)—Z-LA-OC(O)— wherein L⁴ is

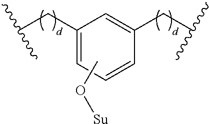

and Su is a hexose form of a monosaccharide and d is an integer independently selected from 1, 2, and 3. In certain embodiments of Formula (III)-(IVB), including any of the foregoing, L³ is —C(O)—NR²—(CR^{a}R^{b})ₐ—C(O)—NR²-L⁴-OC(O)—. In certain embodiments of Formula (III)-(IVB), including any of the foregoing, L³ is —C(O)—NR²—(CH₂)₂—C(O)—NR²-L⁴-OC(O)—. In certain embodiments of Formula (III)-(IVB), including any of the foregoing, L³ is —C(O)—NH—(CR^{a}R^{b})ₐ—C(O)—NH-L⁴-OC(O)—. In certain embodiments of Formula (III)-(IVB), including any of the foregoing, L³ is —C(O)—NH—(CH₂)₂—C(O)—NH-L⁴-OC(O)—.

In certain embodiments of Formula (III)-(IVB), including any of the foregoing, L⁴ is

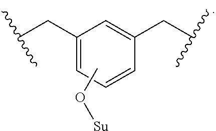

In certain embodiments of Formula (III)-(IVB), including any of the foregoing, L⁴ is

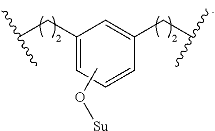

In certain embodiments of Formula (III)-(IVB), including any of the foregoing, L⁴ is

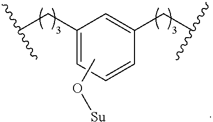

In some embodiments of Formula (III)-(IVB), including any of the foregoing, Su is a sugar moiety. In some embodiments, Su is a hexose form of a monosaccharide. Su may be a glucuronic acid or mannose residue. In certain embodiments of Formula (III)-(IVB), including any of the foregoing, Su is

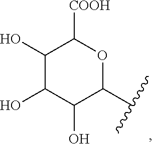 [SMILES: *C1OC(C(=O)O)C(O)C(O)C1O]

wherein

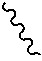

represents attachment to the remainder of the compound. In certain embodiments of Formula (III)-(IVB), including any of the foregoing, Su is

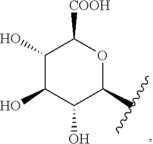 [SMILES: *[C@@H]1O[C@H](C(=O)O)[C@@H](O)[C@H](O)[C@H]1O]

wherein

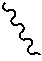

represents attachment to the remainder of the compound.

In certain embodiments of Formula (III)-(IVB), including any of the foregoing, L⁴ is

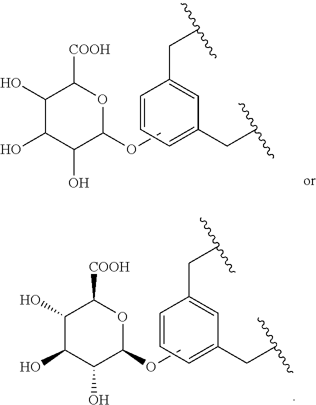

In certain embodiments of Formula (III)-(IVB), including any of the foregoing, L³ is —C(O)—NH—(CH₂)₂—C(O)—NH-L⁴-OC(O)— wherein L⁴ is

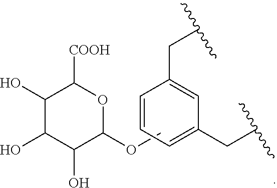

In certain embodiments of Formula (III)-(IVB), including any of the foregoing, L³ is —C(O)—NH—(CH₂)₂—C(O)—NH-L⁴-OC(O)— wherein L⁴ is

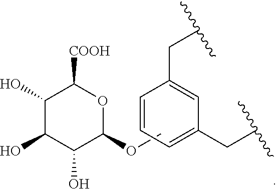

In certain embodiments of Formula (III)-(IVB), including any of the foregoing, L² is —(CR^{a}R^{b})ₐ-POLY¹- and L³ is —C(O)-AA-. In certain embodiments of Formula (III)-(IVB), including any of the foregoing, L² is —(CR^{a}R^{b})ₐ-POLY¹-; L³ is —C(O)-AA-; and, POLY¹ is

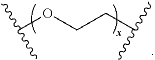

In certain embodiments of Formula (III)-(IVB), including any of the foregoing, L² is —(CH₂)ₐ-POLY¹-; L³ is —C(O)-AA-; POLY¹ is

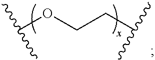

and x is an integer between 10 and 15. In certain embodiments of Formula (III)-(IVB), including any of the foregoing, L² is —(CH₂)₂-POLY¹-; L³ is —C(O)-AA-; and, POLY¹ is

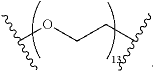

In certain embodiments of Formula (III)-(IVB), including any of the foregoing, L² is —(CH₂)₂-POLY¹-; L³ is —C(O)-AA-; POLY¹ is

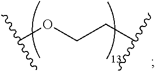

and AA is a dipeptide residue, a tripeptide residue, a tetrapeptide residue, or a pentapeptide residue.

In certain embodiments of Formula (III)-(IVB), including any of the foregoing, L² is —(CR^{a}R^{b})ₐ-POLY¹- and L³ is —C(O)-AA-Z—(CR^{a}R^{b})ₐ—Z—(CR^{a}R^{b})ₐ—C(O)—. In certain embodiments of Formula (III)-(IVB), including any of the foregoing, L² is —(CR^{a}R^{b})ₐ-POLY¹-; L³ is —C(O)-AA-Z—(CR^{a}R^{b})ₐ-Z—(CR^{a}R^{b}) a —C(O)—; POLY¹ is

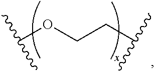

and Z is —NH—. In certain embodiments of Formula (III)-(IVB), including any of the foregoing, L² is —(CH₂)ₐ-POLY¹-; L³ is —C(O)-AA-NH-(CH₂)ₐ-NH-(CH₂) a —C(O)—; POLY¹ is

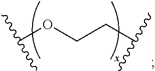

and x is an integer between 10 and 15. In certain embodiments of Formula (III)-(IVB), including any of the foregoing, L² is —(CH₂)₂-POLY¹-; L³ is —C(O)-AA-NH—CH₂—NH—CH₂—C(O)—; and, POLY¹ is

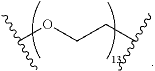

In certain embodiments of Formula (III)-(IVB), including any of the foregoing, L² is —(CH₂)₂-POLY¹-; L³ is —C(O)-AA-NH—CH₂—NH—CH₂—C(O)—; POLY¹ is

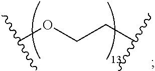

and AA is a dipeptide residue, a tripeptide residue, a tetrapeptide residue, or a pentapeptide residue.

In certain embodiments of Formula (III)-(IVB), including any of the foregoing, L² is —(CR^{a}R^{b})ₐ-POLY¹- and L³ is —C(O). In certain embodiments of Formula (III)-(IVB), including any of the foregoing, L² is —(CR^{a}R^{b})ₐ-POLY¹-; L³ is —C(O); and, POLY¹ is

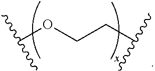

In certain embodiments of Formula (III)-(IVB), including any of the foregoing, L² is —(CH₂)ₐ-POLY¹-; L³ is —C(O); POLY¹ is

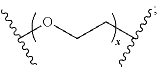

and x is an integer between 10 and 15. In certain embodiments of Formula (III)-(IVB), including any of the foregoing, L² is —(CH₂)₂-POLY¹-; L³ is —C(O); and, POLY¹ is

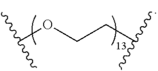

In certain embodiments of Formula (III)-(IVB), including any of the foregoing, L² is —(CH₂)₂-POLY¹-; L³ is —C(O); POLY¹ is

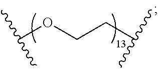

and AA is a dipeptide residue, a tripeptide residue, a tetrapeptide residue, or a pentapeptide residue.

In certain embodiments of Formula (III)-(IVB), including any of the foregoing, L² is —(CR^{a}R^{b})ₐ-POLY¹- and L³ is absent. In certain embodiments of Formula (III)-(IVB), including any of the foregoing, L² is —(CR^{a}R^{b})ₐ-POLY¹-; L³ is absent; and, POLY¹ is

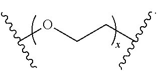

In certain embodiments of Formula (III)-(IVB), including any of the foregoing, L² is —(CH₂)ₐ-POLY¹-; L³ is absent; POLY¹ is

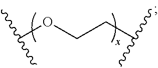

and x is an integer between 10 and 15. In certain embodiments of Formula (III)-(IVB), including any of the foregoing, L² is —(CH₂)₂-POLY¹-; L³ is absent; and, POLY¹ is

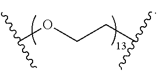

In certain embodiments of Formula (III)-(IVB), including any of the foregoing, L² is —(CH₂)₂—POLY¹-; L³ is absent; POLY¹ is

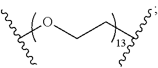

and AA is a dipeptide residue, a tripeptide residue, a tetrapeptide residue, or a pentapeptide residue.

In certain embodiments of Formula (III)-(IVB), including any of the foregoing, L² is —(CR^{a}R^{b})ₐ-POLY¹- and L³ is —C(O)-AA-Z—(CR^{a}R^{b})ₐ—. In certain embodiments of Formula (III)-(IVB), including any of the foregoing, L² is —(CR^{a}R^{b})ₐ-POLY¹-; L³ is —C(O)-AA-Z—(CR^{a}R^{b})ₐ—; and, POLY¹ is

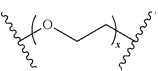

In certain embodiments of Formula (III)-(IVB), including any of the foregoing, L² is —(CH₂)ₐ-POLY¹-; —C(O)-AA-NR²—(CR^{a}R^{b})ₐ-; POLY¹ is

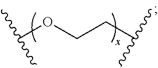

and x is an integer between 10 and 15. In certain embodiments of Formula (III)-(IVB), including any of the foregoing, L² is —(CH₂)₂-POLY¹-; L³ is —C(O)-AA-NH—(CR^{a}R^{b})ₐ—; and, POLY¹ is

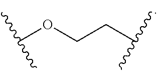 [SMILES: *CCO*]

In certain embodiments of Formula (III)-(IVB), including any of the foregoing, L² is —(CH₂)₂-POLY¹-; L³ is —C(O)-AA-NH—(CR^{a}R^{b})ₐ-; POLY¹ is

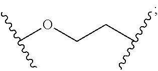 [SMILES: *CCO*]

and AA is a dipeptide residue, a tripeptide residue, a tetrapeptide residue, or a pentapeptide residue.

Non-limiting examples of -L²-L³- include:

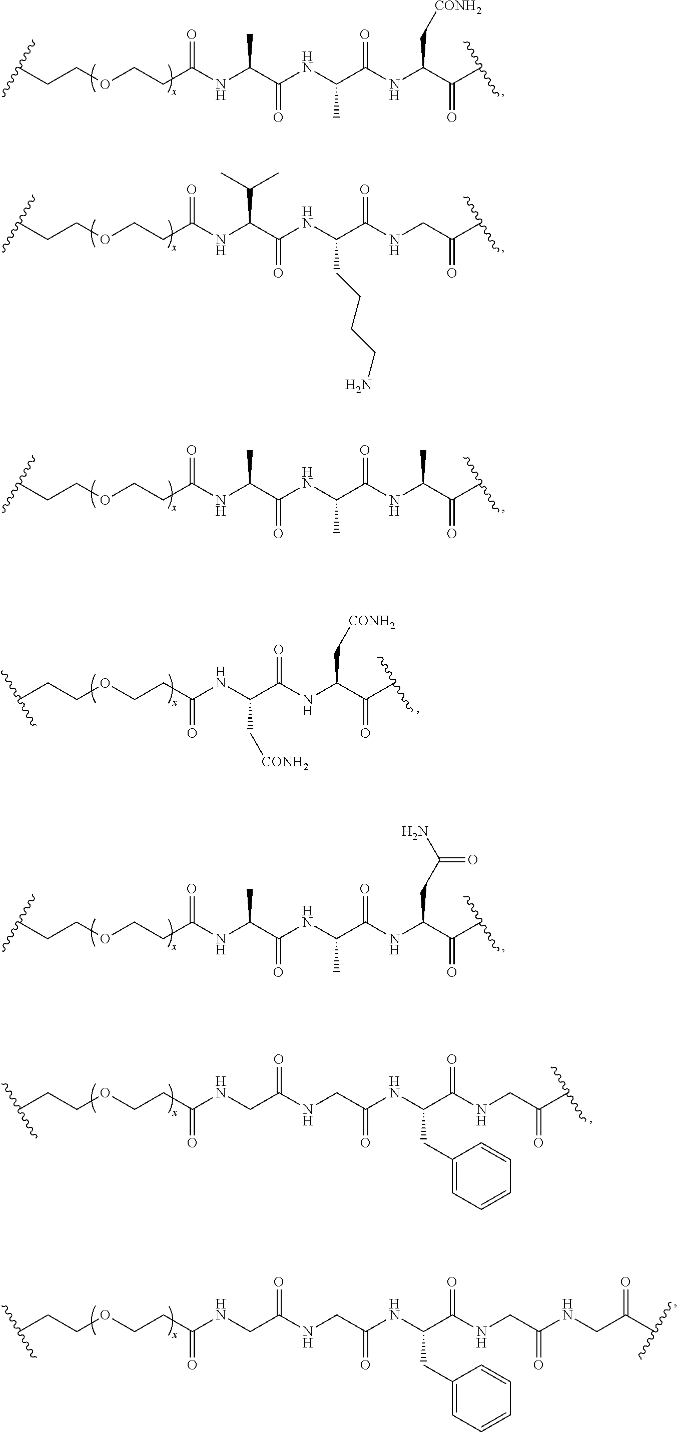

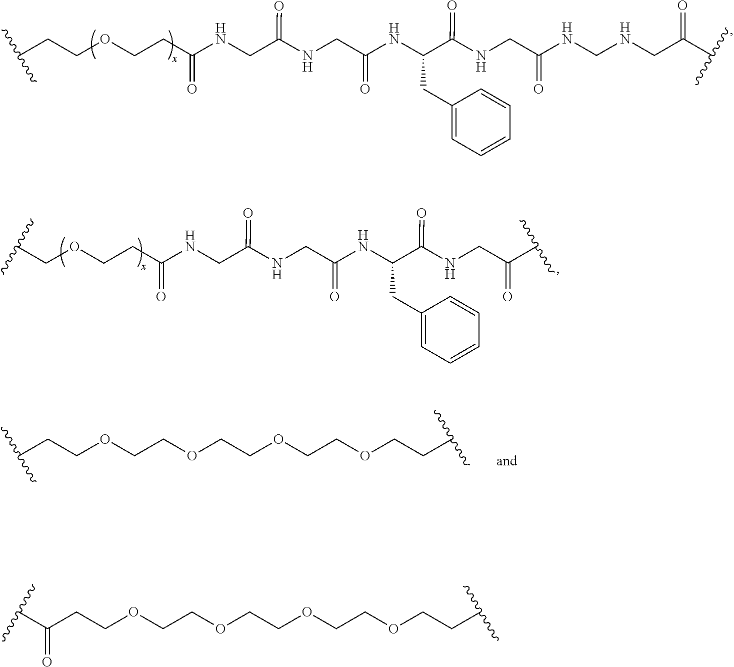

Additional non-limiting examples of -L²-L³- include:

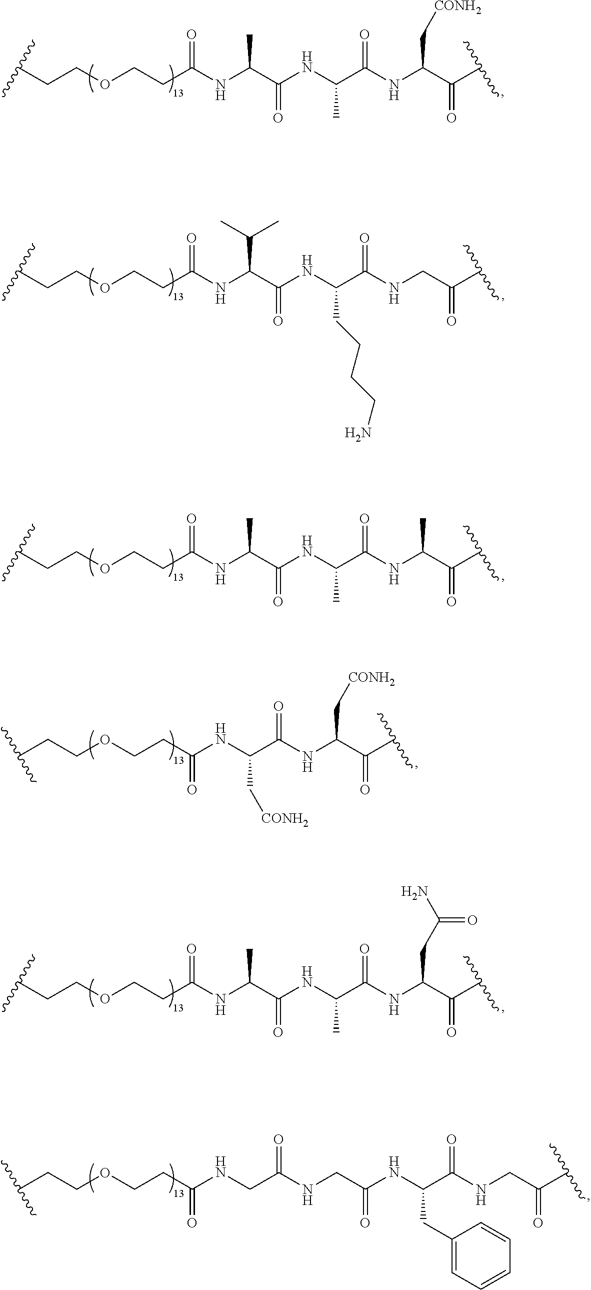

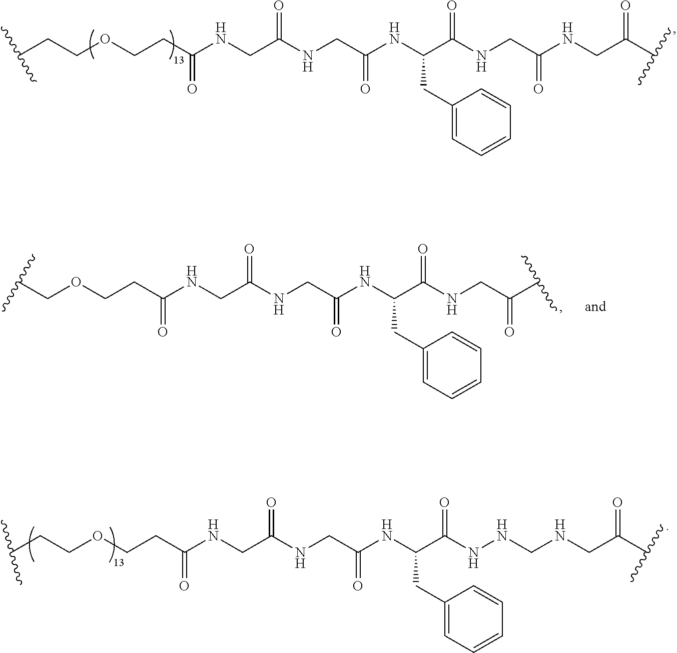

In certain embodiments of Formula (III)-(IVB), including any of the foregoing, L² is

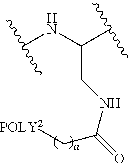

and L³ is —C(O)—Z—(CR^{a}R^{b}) a —C(O)—Z-L⁴-OC(O)— wherein L⁴ is

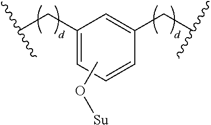

and Su is a hexose form of a monosaccharide and d is an integer independently selected from 1, 2, and 3. In certain embodiments of Formula (III)-(IVB), including any of the foregoing, L² is

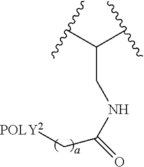

and L³ is —C(O)—Z—(CR^{a}R^{b}) a —C(O)—Z-L⁴-OC(O)—.

In certain embodiments of Formula (III)-(IVB), including any of the foregoing, L² is

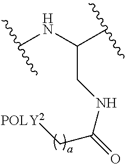

and L³ is —C(O). In certain embodiments, including any of the foregoing, L² is

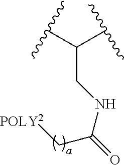

and L³ is absent.

In certain embodiments of Formula (III)-(IVB), including any of the foregoing, L² is selected from the group consisting of

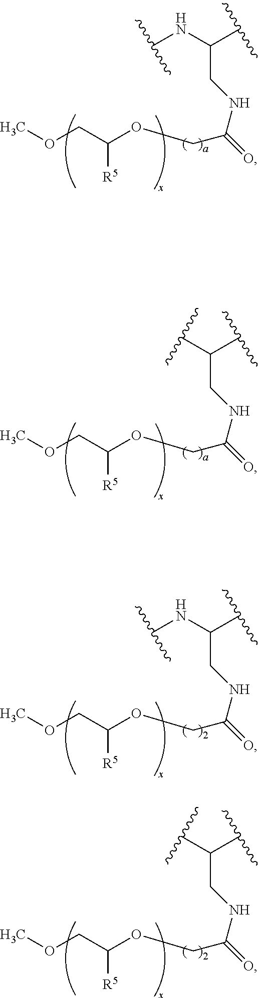

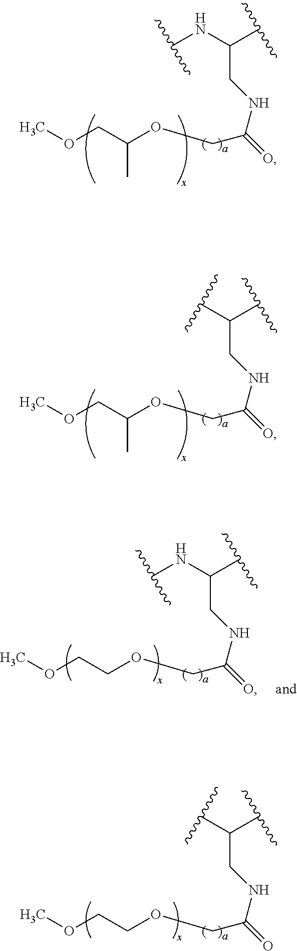

and L³ is —C(O)—NH—(CR^{a}R^{b}) a —C(O)—NH-L⁴-OC(O)—.

In certain embodiments of Formula (III)-(IVB), including any of the foregoing, L² is selected from the group consisting of

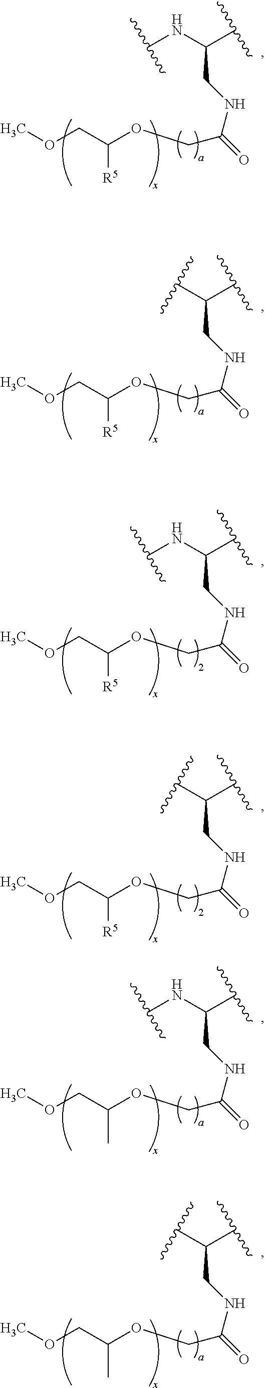

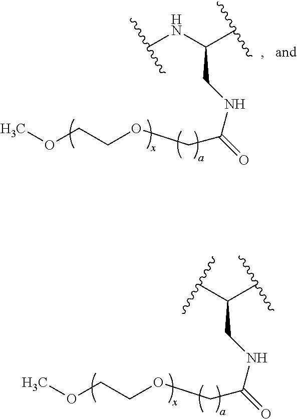

and L³ is —C(O)—NH—(CR^{a}R^{b}) a —C(O)—NH-L⁴-OC(O)—.

In certain embodiments of Formula (III)-(IVB), including any of the foregoing, L² is selected from the group consisting of

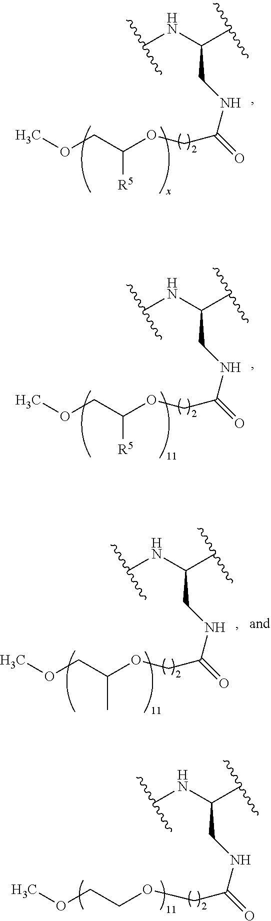

and L³ is —C(O)—NH—(CH₂)₂—C(O)—NH-L⁴-OC(O)— wherein L⁴ is

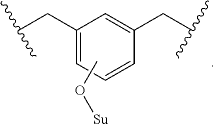

In certain embodiments of Formula (III)-(IVB), including any of the foregoing, L² is selected from the group consisting of

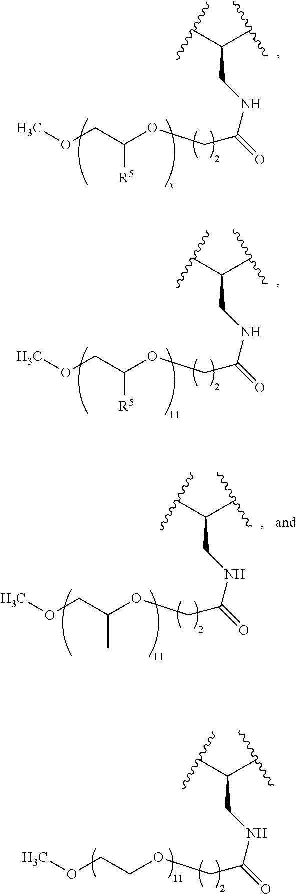

### and L³ is —C(O).

In certain embodiments, including any of the foregoing, D is a cytotoxic payload selected from a tubulin inhibitor, a DNA topoisomerase I inhibitor, and a DNA topoisomerase II inhibitor. In some embodiments, including any of the foregoing, D is a tubulin inhibitor. In some embodiments, including any of the foregoing, D is a DNA topoisomerase I inhibitor. In some embodiments, including any of the foregoing, D is a DNA topoisomerase I inhibitor selected from the group consisting of irinotecan, SN-38, topotecan, exatecan. In some embodiments, including any of the foregoing, D is irinotecan. In some embodiments, including any of the foregoing, D is SN-38. In some embodiments, including any of the foregoing, D is topotecan. In some embodiments, including any of the foregoing, D is exatecan. In some embodiments, including any of the foregoing, D is a DNA topoisomerase II inhibitor. In some embodiments, including any of the foregoing, D is a DNA topoisomerase II inhibitor selected from the group consisting of etoposide, teniposide, and tafluposide. In some embodiments, including any of the foregoing, D is etoposide. In some embodiments, including any of the foregoing, D is teniposide. In some embodiments, including any of the foregoing, D is tafluposide. In some embodiments, including any of the foregoing, D is selected from the group consisting of hemiasterlins, camptothecins, and anthracyclines. Anthracyclines may include PNU-159682 and EDA PNU-159682 derivatives. In some embodiments, including any of the foregoing, D is an anthracycline selected from the group consisting of daunorubicin, doxorubicin, epirubicin, idarubicin, mitoxantrone, and valrubicin. In some embodiments, including any of the foregoing, D is daunorubicin. In some embodiments, including any of the foregoing, D is doxorubicin. In some embodiments, including any of the foregoing, D is epirubicin. In some embodiments, including any of the foregoing, D is idarubicin. In some embodiments, including any of the foregoing, D is mitoxantrone. In some embodiments, including any of the foregoing, D is valrubicin. In some embodiments, including any of the foregoing, D is a hemiasterlin. In some embodiments, including any of the foregoing, D is a camptothecin. In some embodiments, including any of the foregoing, D is an anthracycline. In some embodiments, including any of the foregoing, D is PNU-159682. In some embodiments, including any of the foregoing, D is an EDA PNU compound. In some embodiments, including any of the foregoing, D is an EDA PNU-159682 derivative. In some embodiments, including any of the foregoing, D is hemiasterlin, exatecan, PNU-159682, or an EDA PNU-159682 derivative. In some embodiments, including any of the foregoing, D is hemiasterlin. In some embodiments, including any of the foregoing, D is exatecan. In some embodiments, including any of the foregoing, D is of PNU-159682. In some embodiments, including any of the foregoing, D is EDA PNU-159682 compound or derivative. In some embodiments, including any of the foregoing, D is not an immunestimulatory compound.

In some embodiments, including any of the foregoing, D is an alkylating agent. In some embodiments, including any of the foregoing, D is a bifunctional alkylator. In some embodiments, including any of the foregoing, D is a bifunctional alkylator selected from the group consisting of cyclophosphamide, mechlorethamine, chlorambucil, and melphalan. In some embodiments, including any of the foregoing, D is cyclophosphamide. In some embodiments, including any of the foregoing, D is mechlorethamine. In some embodiments, including any of the foregoing, D is chlorambucil. In some embodiments, including any of the foregoing, D is melphalan. In some embodiments, including any of the foregoing, D is a monofunctional alkylator. In some embodiments, including any of the foregoing, D is a monofunctional alkkylator selected from the group consisting of dacabazine, nitrosourea, and temozolomide. In some embodiments, including any of the foregoing, D is dacabazine. In some embodiments, including any of the foregoing, D is nitrosourea. In some embodiments, including any of the foregoing, D is temozolomide. In some embodiments, including any of the foregoing, D is a cytoskeletal disruptor (e.g., a taxane). In some embodiments, including any of the foregoing, D is a cytoskeletal disruptor selected from the group consisting of paclitaxel, docetaxel, abraxane, and taxotere. In some embodiments, including any of the foregoing, D is paclitaxel. In some embodiments, including any of the foregoing, D is docetaxel. In some embodiments, including any of the foregoing, D is abraxane. In some embodiments, including any of the foregoing, D is taxotere. In some embodiments, including any of the foregoing, D is an epothilone. In some embodiments, including any of the foregoing, D is an epothilone selected from the group consisting of epothilone A, epothilone B, epothilone C, epothilone D, and ixabepilone. In some embodiments, including any of the foregoing, D is epothilone A. In some embodiments, including any of the foregoing, D is epothilone B. In some embodiments, including any of the foregoing, D is epothilone C. In some embodiments, including any of the foregoing, D is epothilone D. In some embodiments, including any of the foregoing, D is ixabepilone. In some embodiments, including any of the foregoing, D is a histone deacetylase inhibitor. In some embodiments, including any of the foregoing, D is a histone deacetylase inhibitor selected from the group consisting of vorinostat and romidepsin. In some embodiments, including any of the foregoing, D is vorinostat. In some embodiments, including any of the foregoing, D is romidepsin. In some embodiments, including any of the foregoing, D is a kinase inhibitor. In some embodiments, including any of the foregoing, D is a kinase inhibitor selected from the group consisting of bortezomib, erlotinib, gefitinib, imatinib, vemurafenib, and vismodegib. In some embodiments, including any of the foregoing, D is bortezomib. In some embodiments, including any of the foregoing, D is erlotinib. In some embodiments, including any of the foregoing, D is gefitinib. In some embodiments, including any of the foregoing, D is imatinib. In some embodiments, including any of the foregoing, D is vemurafenib. In some embodiments, including any of the foregoing, D is vismodegib. In some embodiments, including any of the foregoing, D is a nucleotide analog and/or precursor analog. In some embodiments, including any of the foregoing, D is a nucleotide analog and/or precursor analog selected from the group consisting of azacitidine, azathioprine, capecitabine, cyatarabine, doxifluridine, fluorouracil, gemcitabine, hydroxyurea, mercaptopurine, methotrexate, and tioguanine (formerly thioguanine). In some embodiments, including any of the foregoing, D is azacitidine. In some embodiments, including any of the foregoing, D is azathioprine. In some embodiments, including any of the foregoing, D is capecitabine. In some embodiments, including any of the foregoing, D is cyatarabine. In some embodiments, including any of the foregoing, D is doxifluridine. In some embodiments, including any of the foregoing, D is fluorouracil. In some embodiments, including any of the foregoing, D is gemcitabine. In some embodiments, including any of the foregoing, D is hydroxyurea. In some embodiments, including any of the foregoing, D is mercaptopurine. In some embodiments, including any of the foregoing, D is methotrexate. In some embodiments, including any of the foregoing, D is tioguanine (formerly thioguanine). In some embodiments, including any of the foregoing, D is a peptide antibiotic. In some embodiments, including any of the foregoing, D is a peptide antibiotic selected from the group consisting of bleomycin and actinomycin. In some embodiments, including any of the foregoing, D is bleomycin. In some embodiments, including any of the foregoing, D is actinomycin. In some embodiments, including any of the foregoing, D is a platinum-based agent. In some embodiments, including any of the foregoing, D is a platinum-based agent selected from the group consisting of carboplatin, cisplatin, and oxaliplatin. In some embodiments, including any of the foregoing, D is carboplatin. In some embodiments, including any of the foregoing, D is cisplatin. In some embodiments, including any of the foregoing, D is oxaliplatin. In some embodiments, including any of the foregoing, D is a retinoid. In some embodiments, including any of the foregoing, D is a retinoid selected from the group consisting of tretinoin, alitretinoin, and bexarotene. In some embodiments, including any of the foregoing, D is tretinoin. In some embodiments, including any of the foregoing, D is alitretinoin. In some embodiments, including any of the foregoing, D is bexarotene. In some embodiments, including any of the foregoing, D is a vinca alkaloid and derivatives thereof. In some embodiments, including any of the foregoing, D is a vinca alkaloid and derivatives thereof selected from the group consisting of vinblastine, vincristine, vindesine, vinorelbine. In some embodiments, including any of the foregoing, D is a residue of vinblastine. In some embodiments, including any of the foregoing, D is vincristine. In some embodiments, including any of the foregoing, D is vindesine.

In any of the foregoing embodiments, the conjugate comprises n2 number of linker-payloads, wherein n2 is an integer from 1 to 10. In some embodiments, n2 is 2. In some embodiments, n2 is 3. In some embodiments, n2 is 4. In some embodiments, n2 is 5. In some embodiments, n2 is 6. In some embodiments, n2 is 7. In some embodiments, n2 is 8. In some embodiments, n2 is 9. In some embodiments, n2 is 10.

In some embodiments, provided herein are anti-ROR1 conjugates having the structure of any of Conjugates A-MM in the table below. In some embodiments, n2 is an integer from 1 to 8. In some embodiments, n2 is 2. In some embodiments, n2 is 4. In some embodiments, n2 is 6. In some embodiments, n2 is 8. The present disclosure encompasses each and every regioisomer of the conjugate structures depicted below.

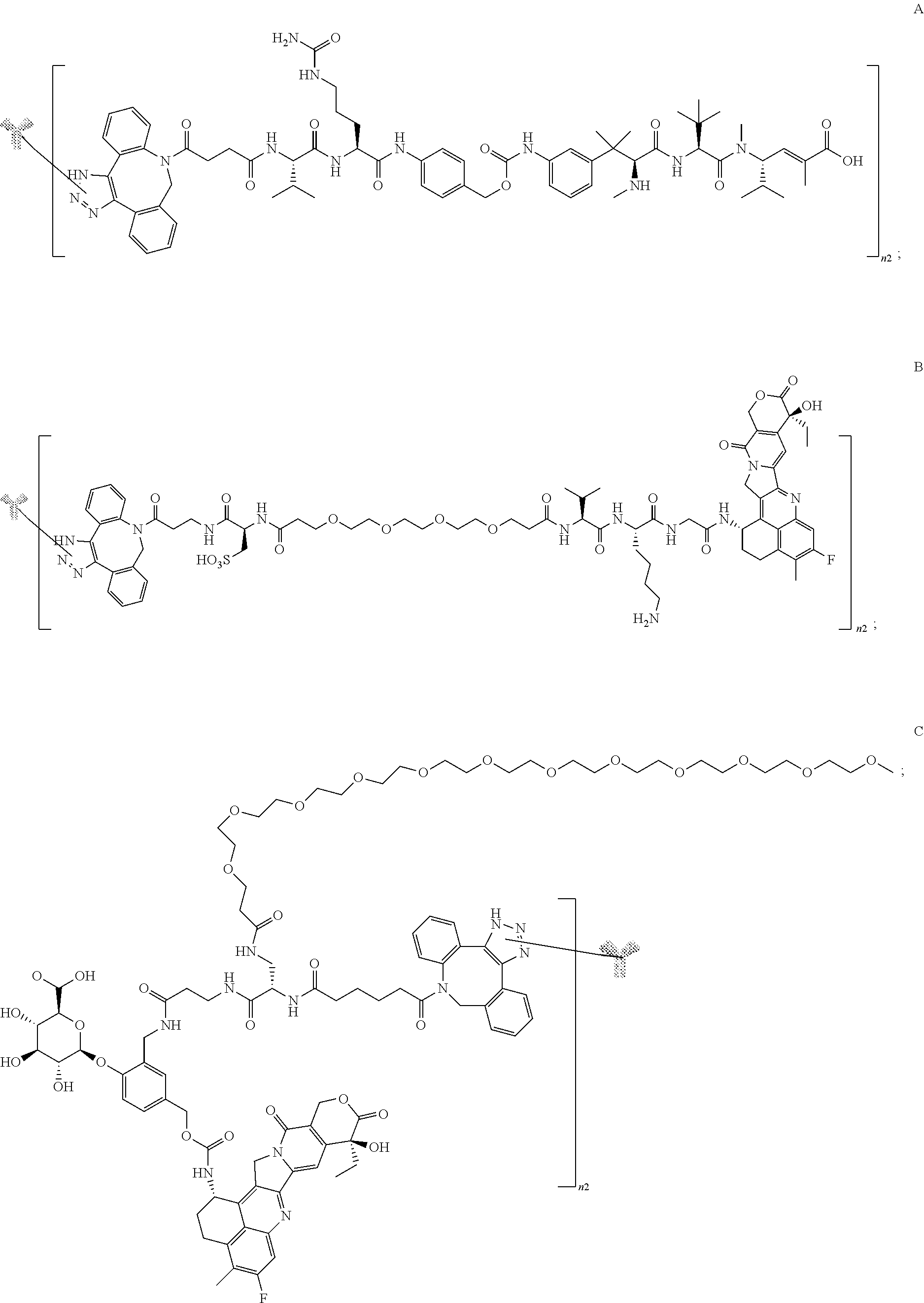

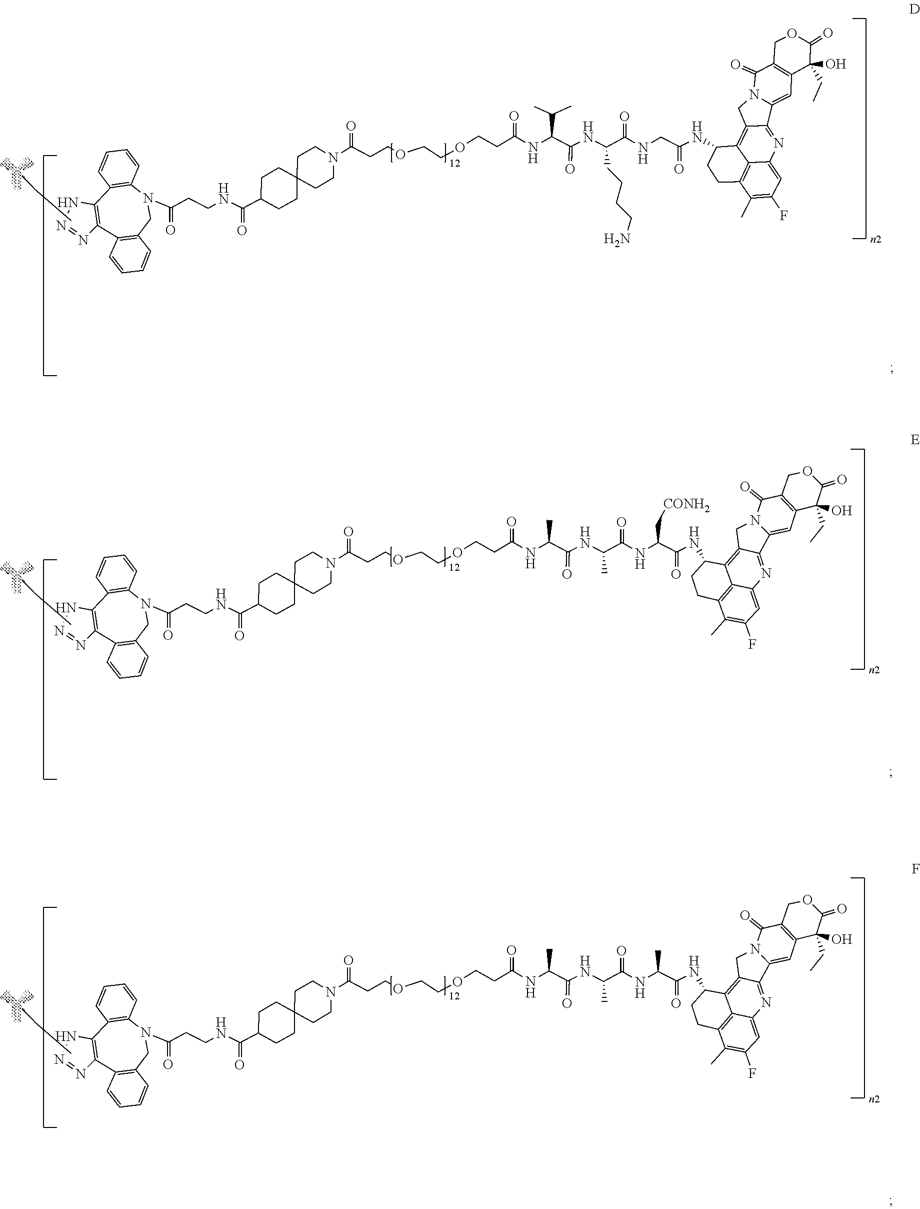

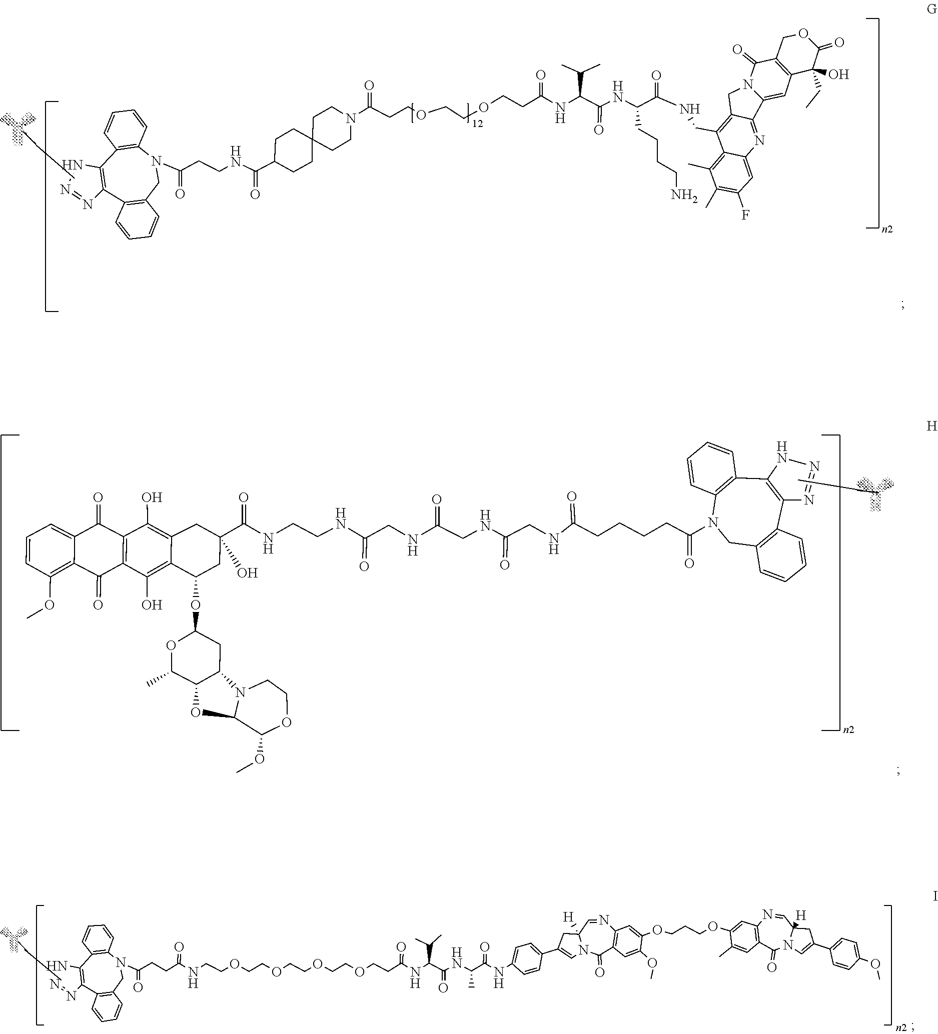

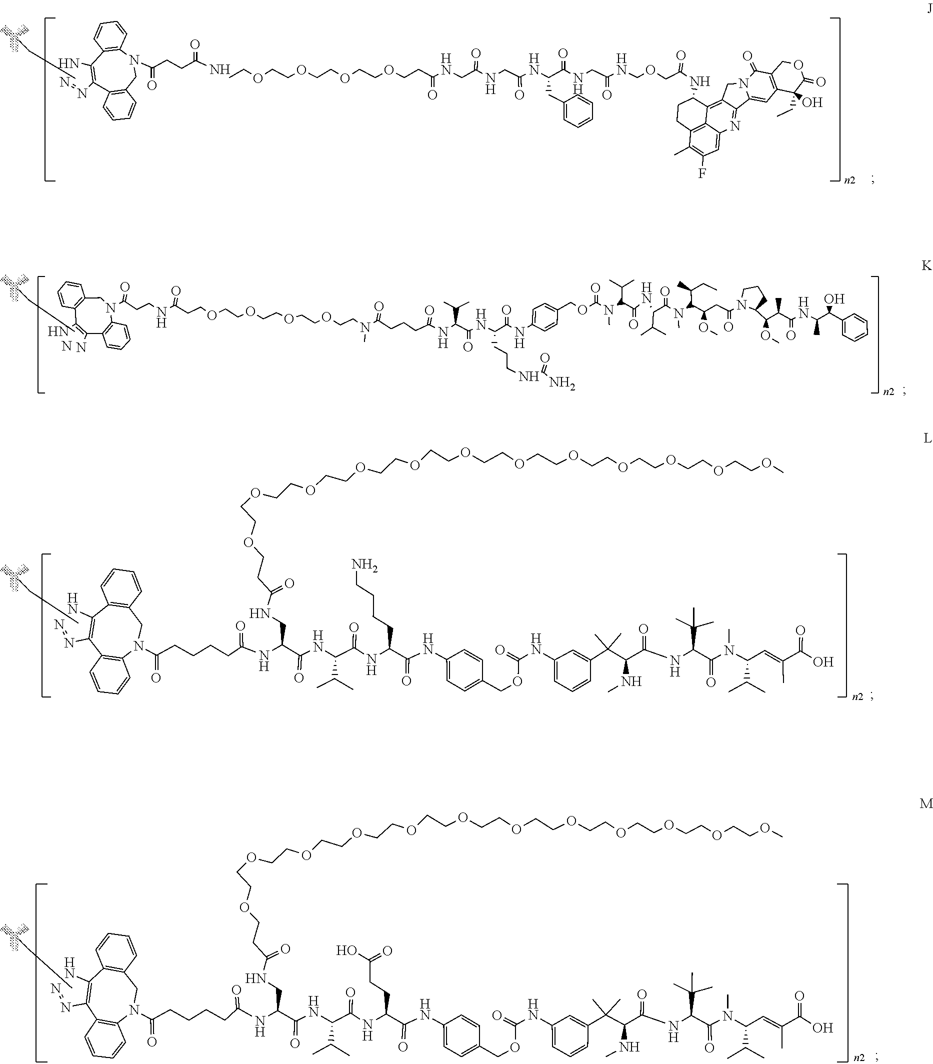

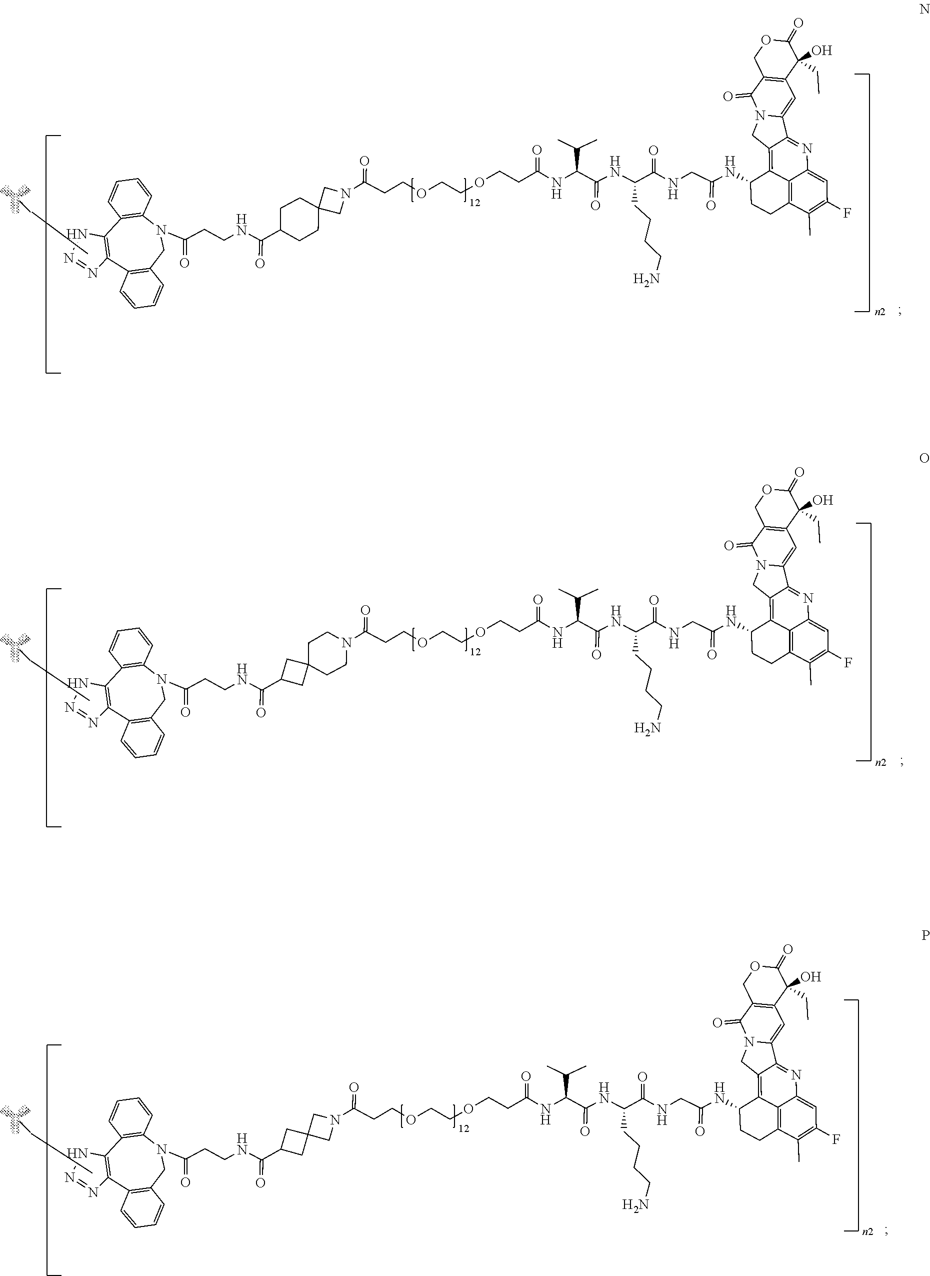

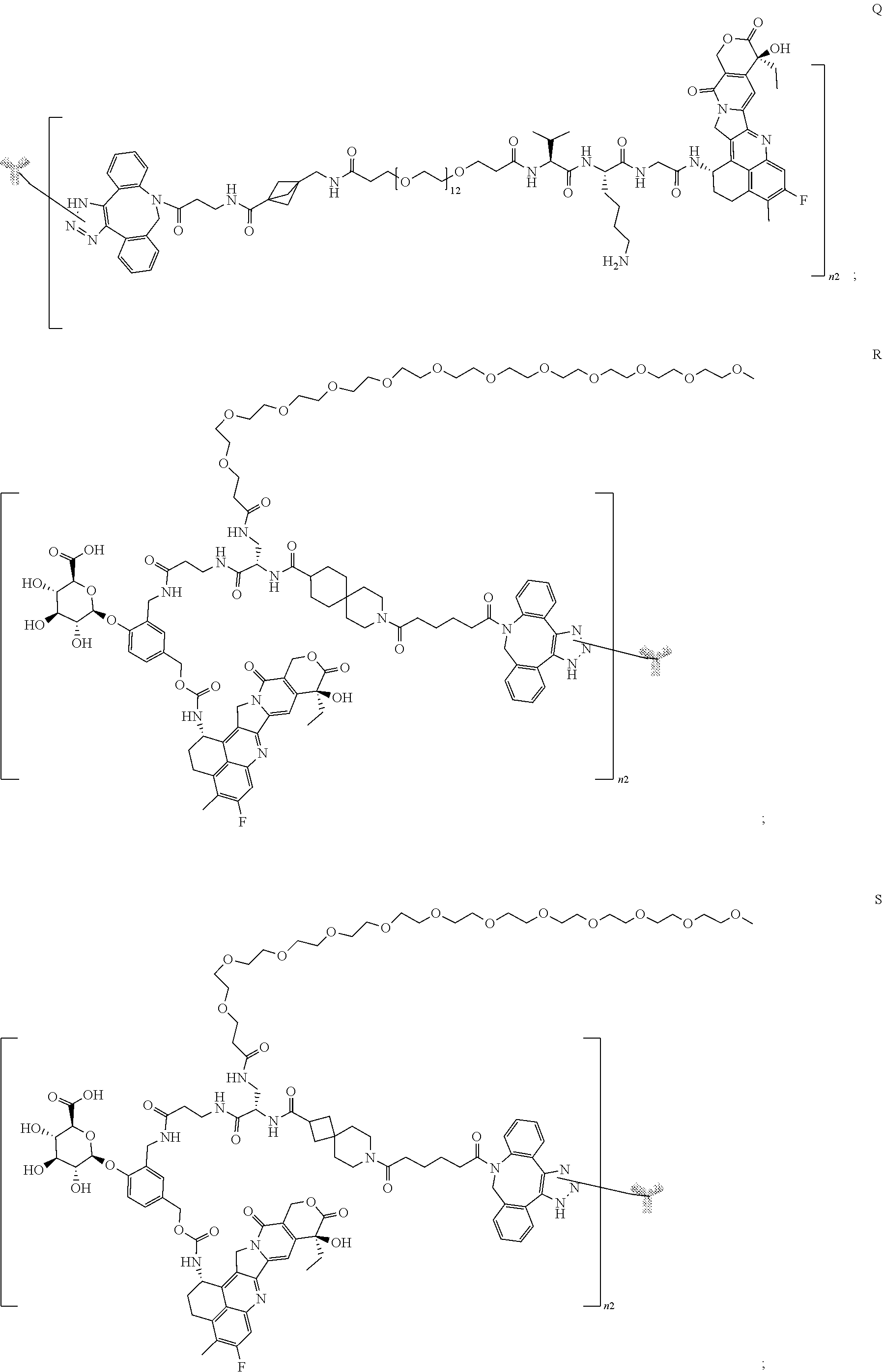

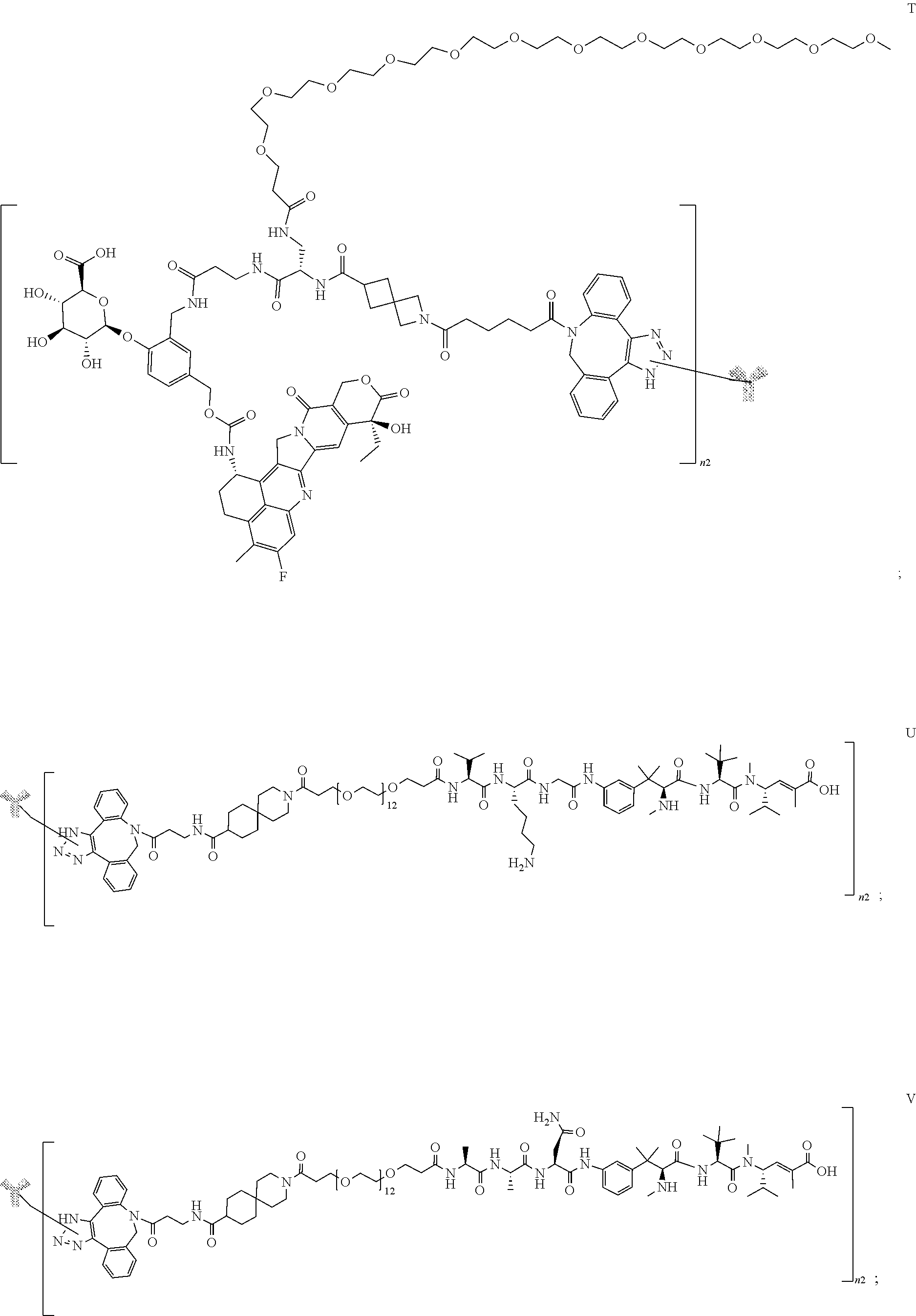

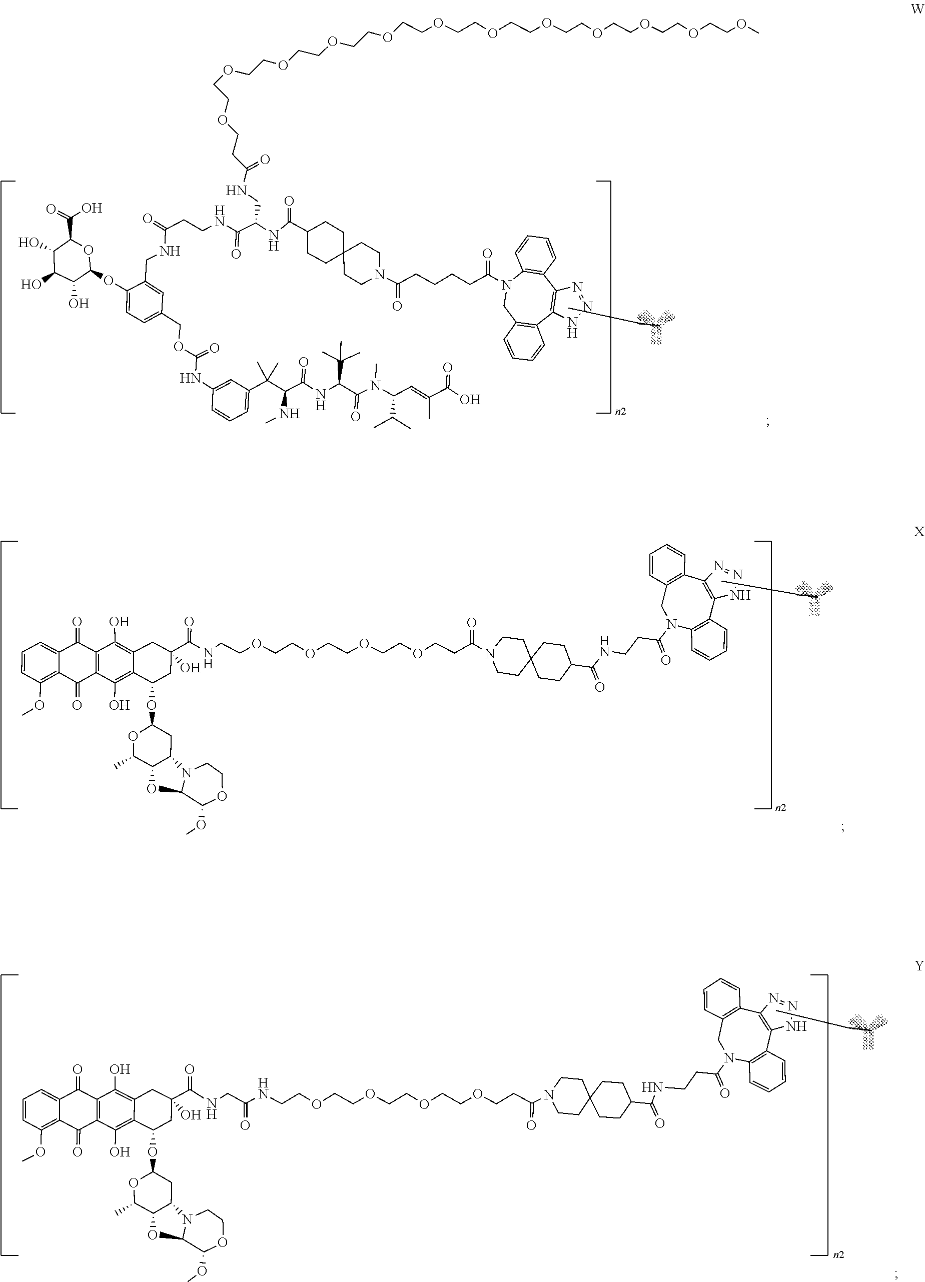

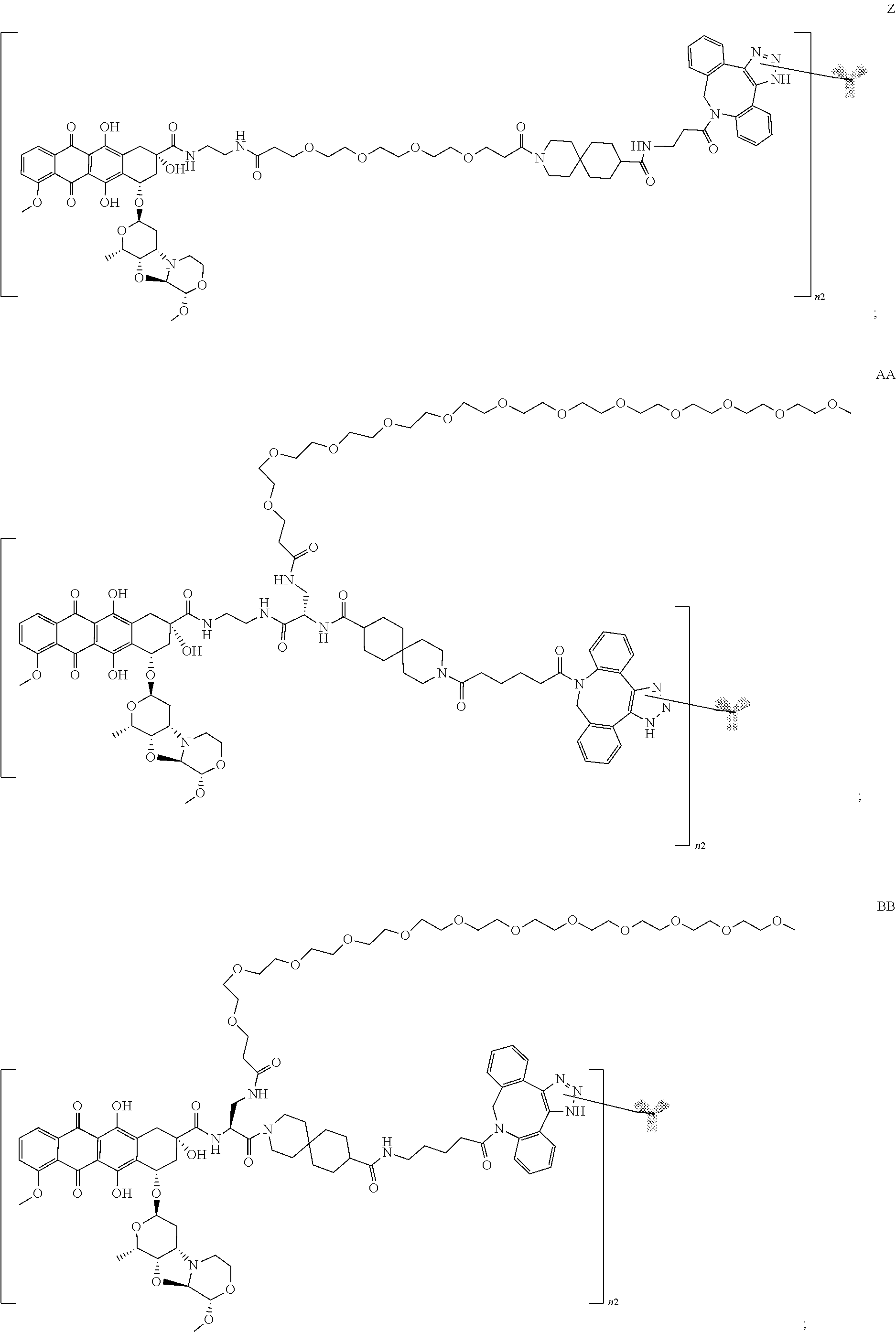

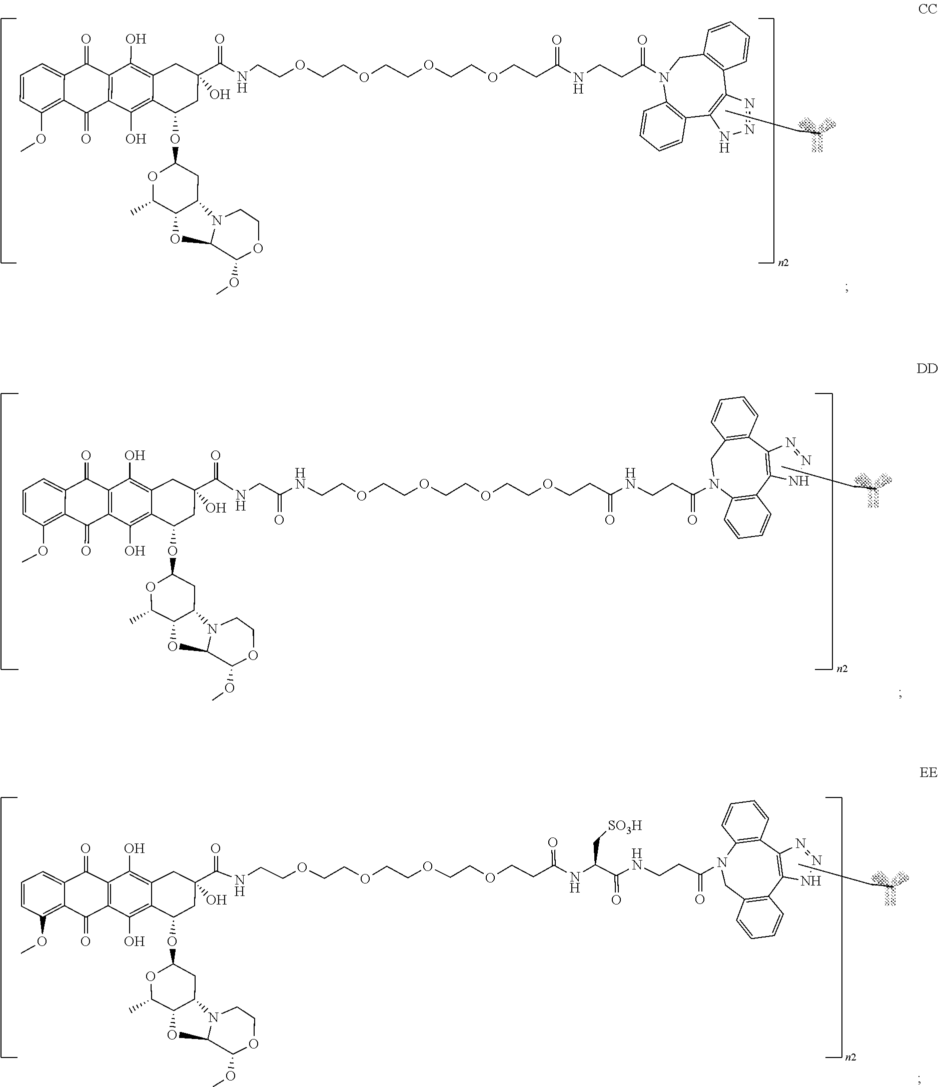

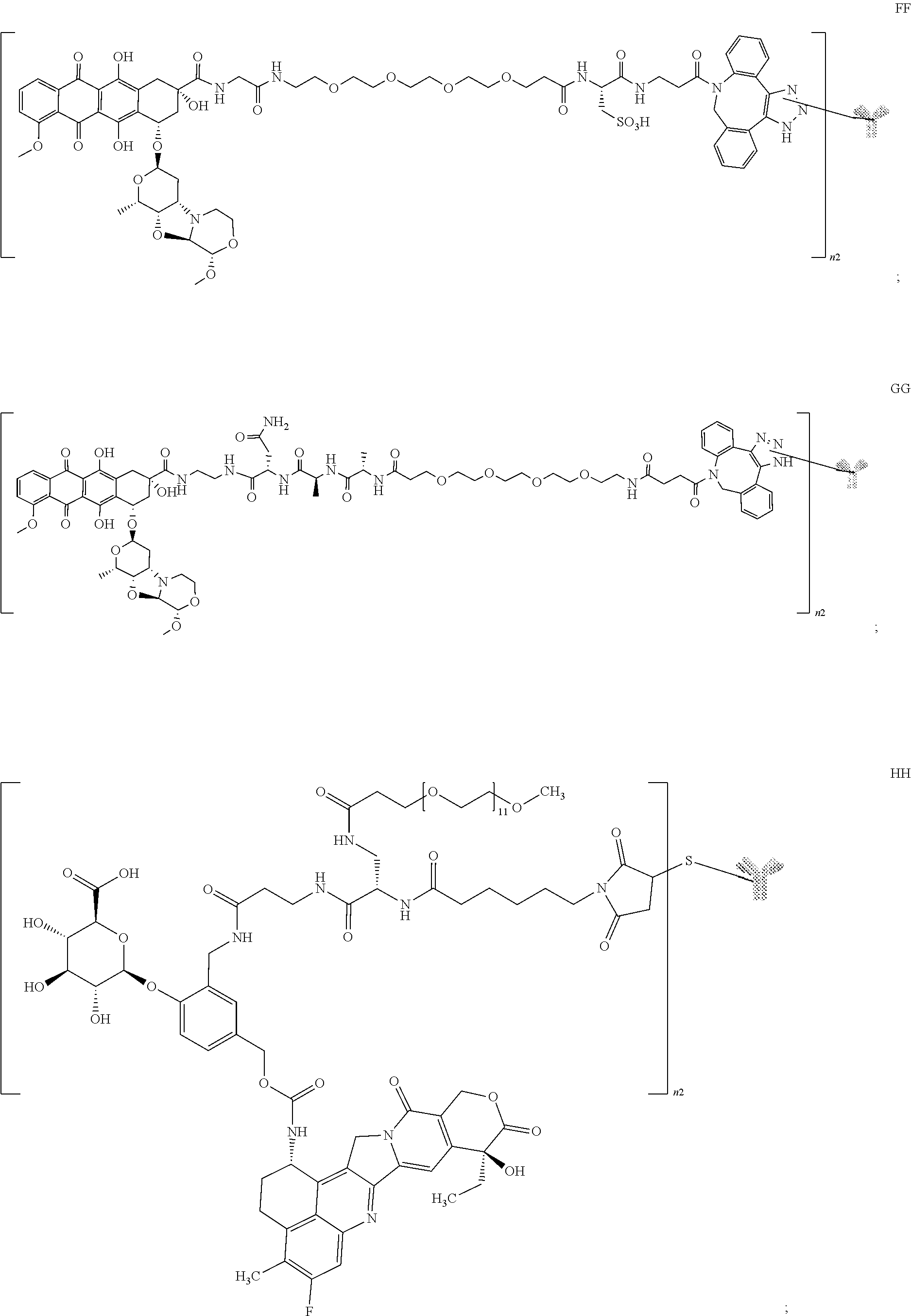

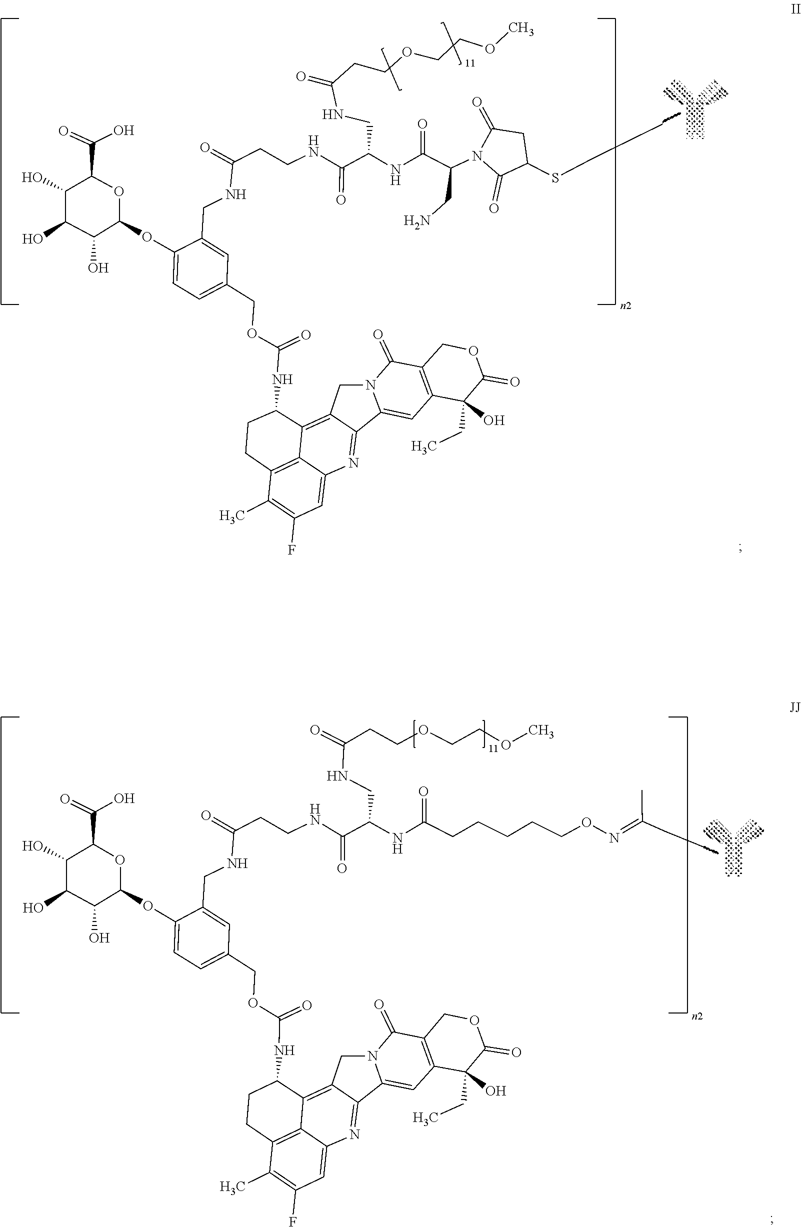

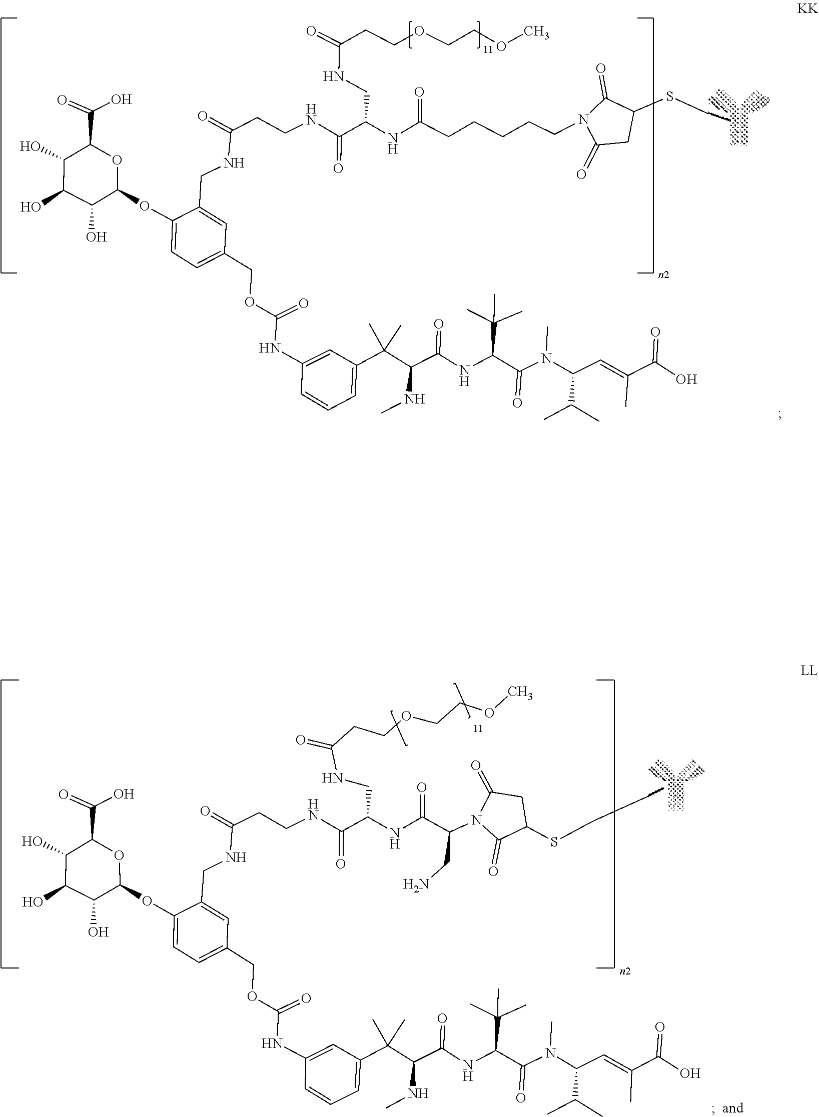

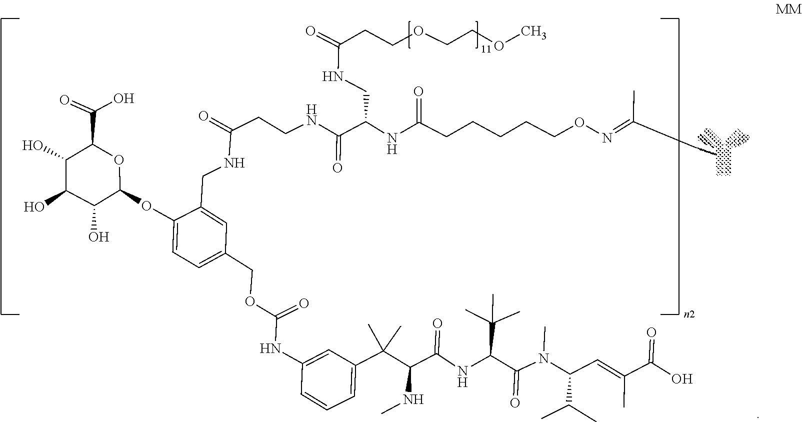

In some embodiments, provided herein are anti-ROR1 conjugates having the structure of any of Conjugates A1-MM1 in the table below. In some embodiments, n2 is an integer from 1 to 8. In some embodiments, n2 is 2. In some embodiments, n2 is 4. In some embodiments, n2 is 6. In some embodiments, n2 is 8. The present disclosure encompasses each and every regioisomer of the conjugate structures depicted below.

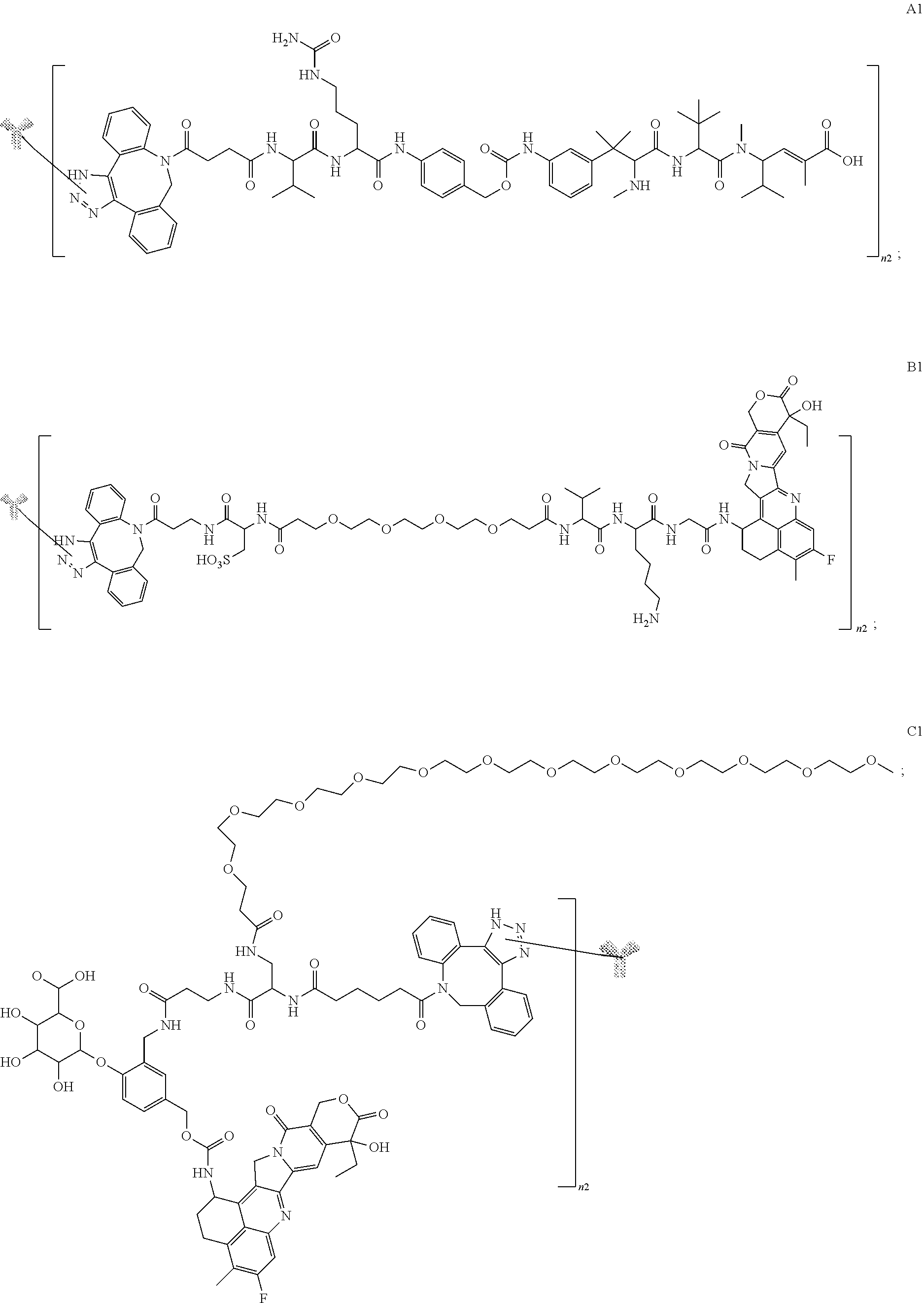

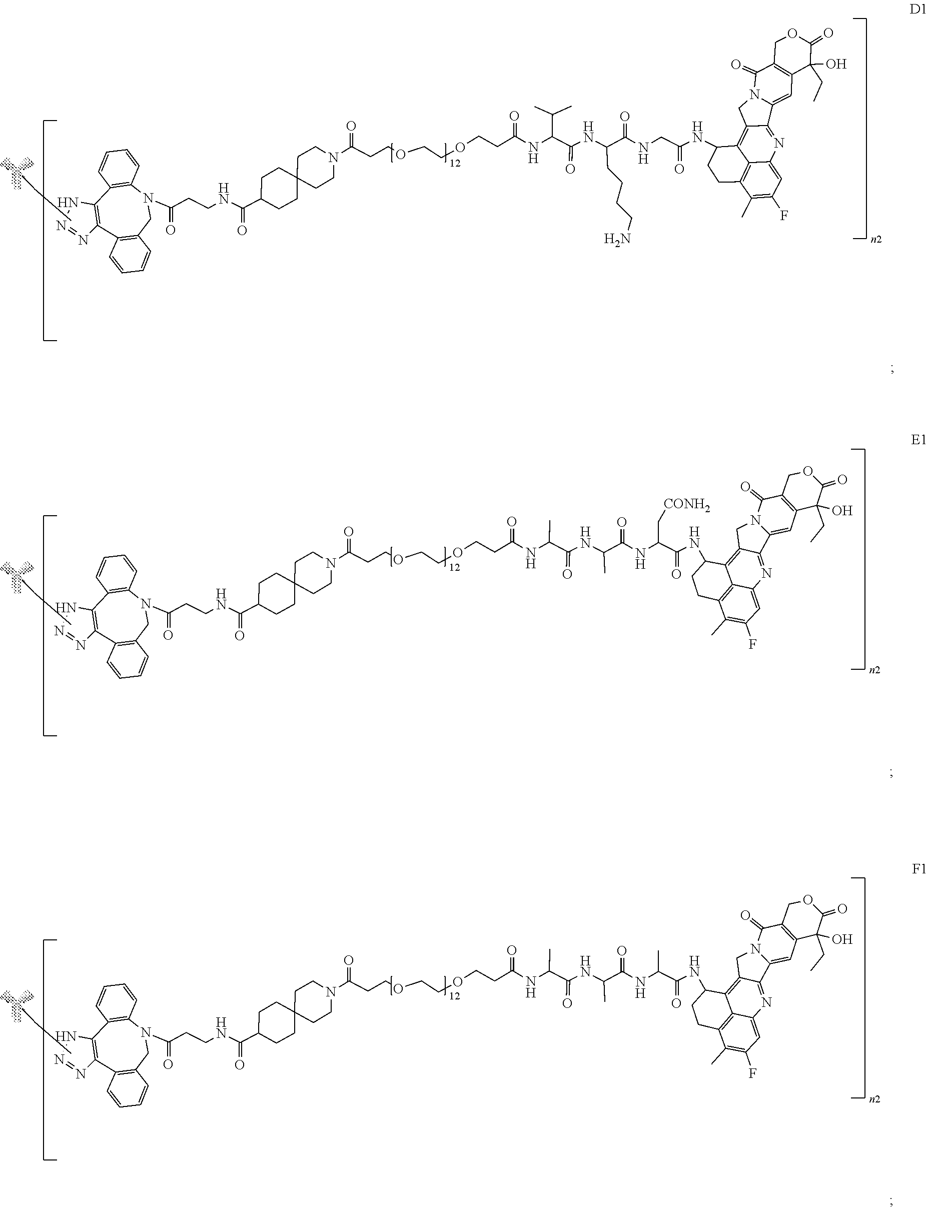

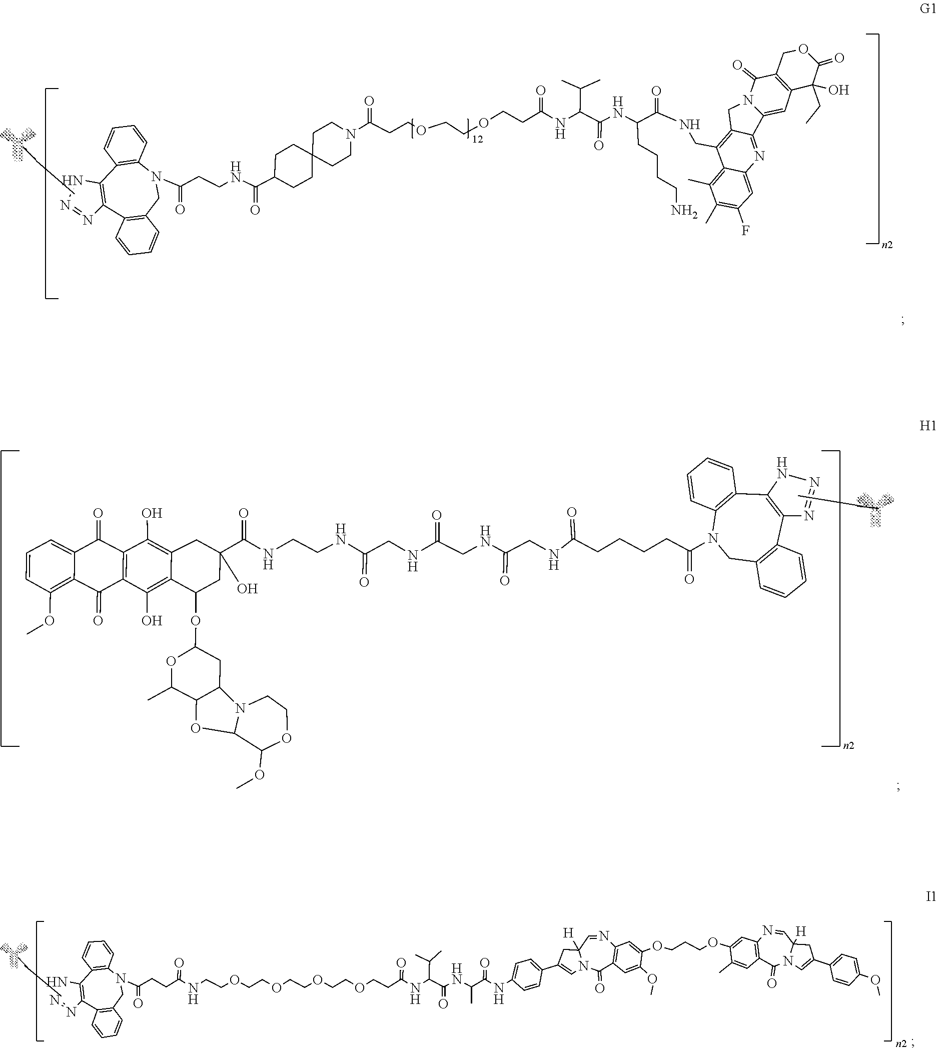

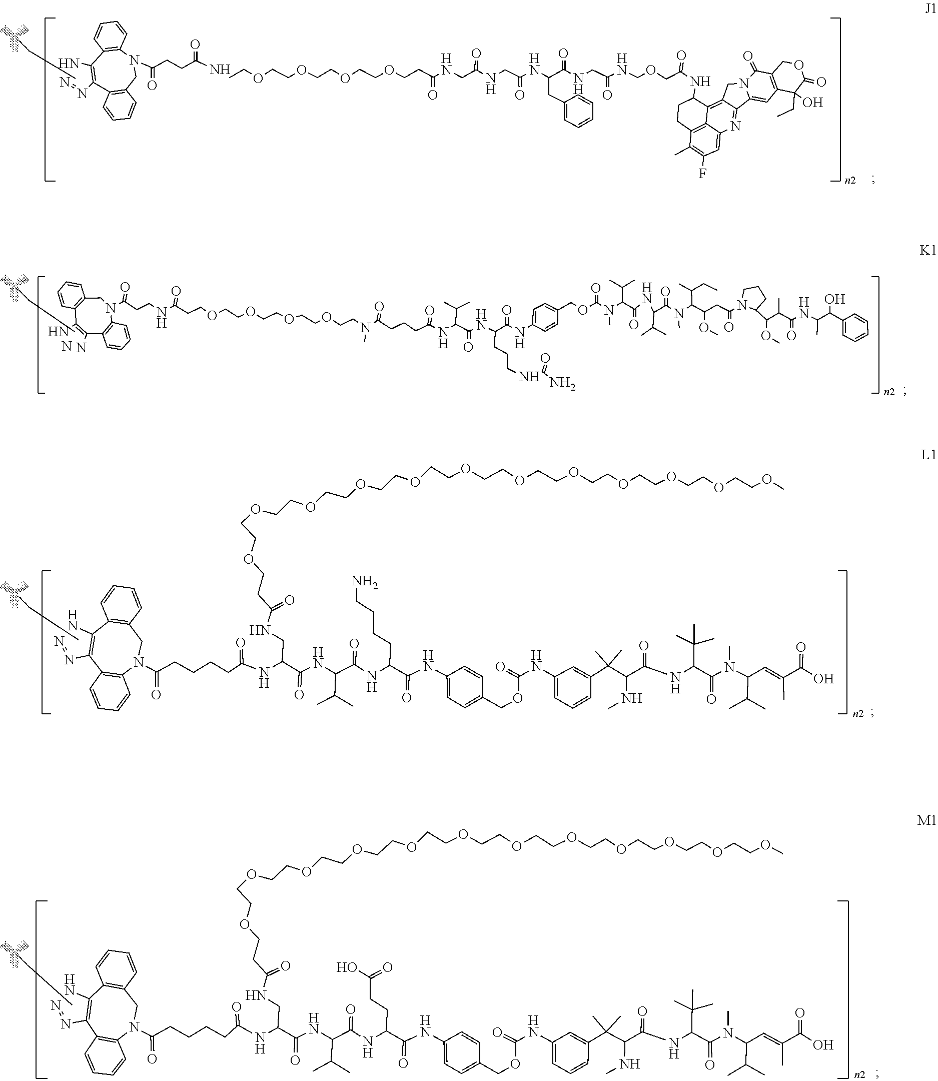

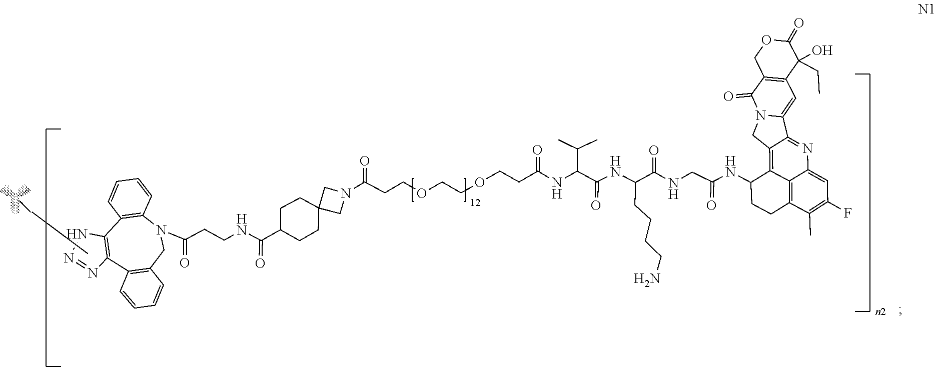

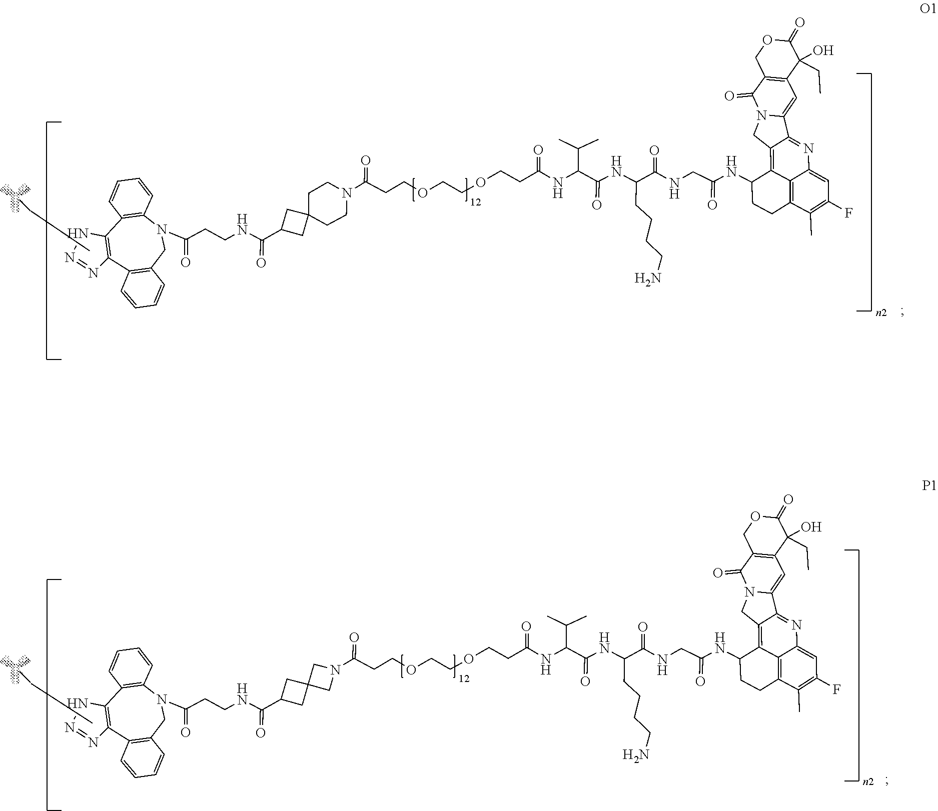

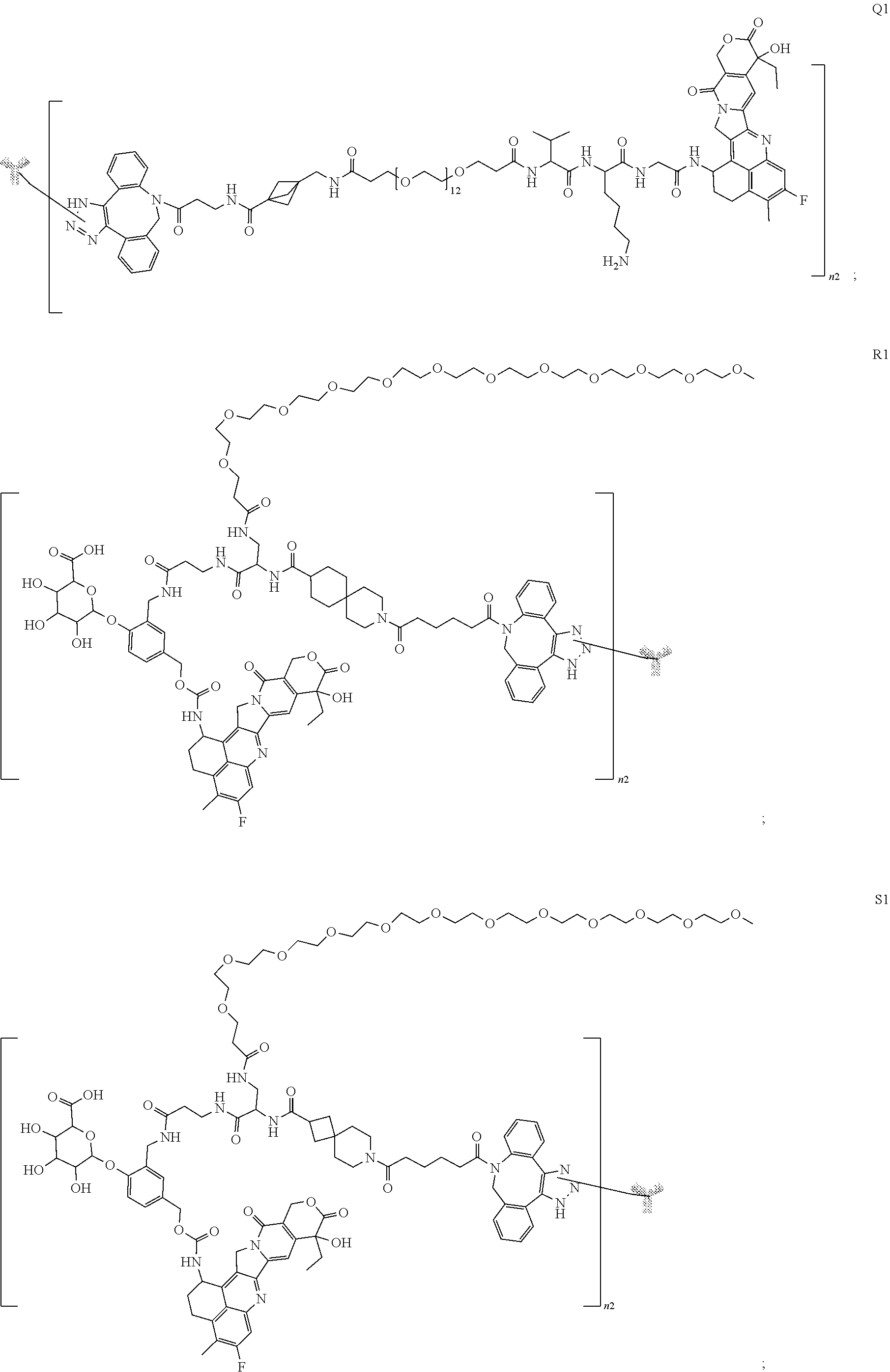

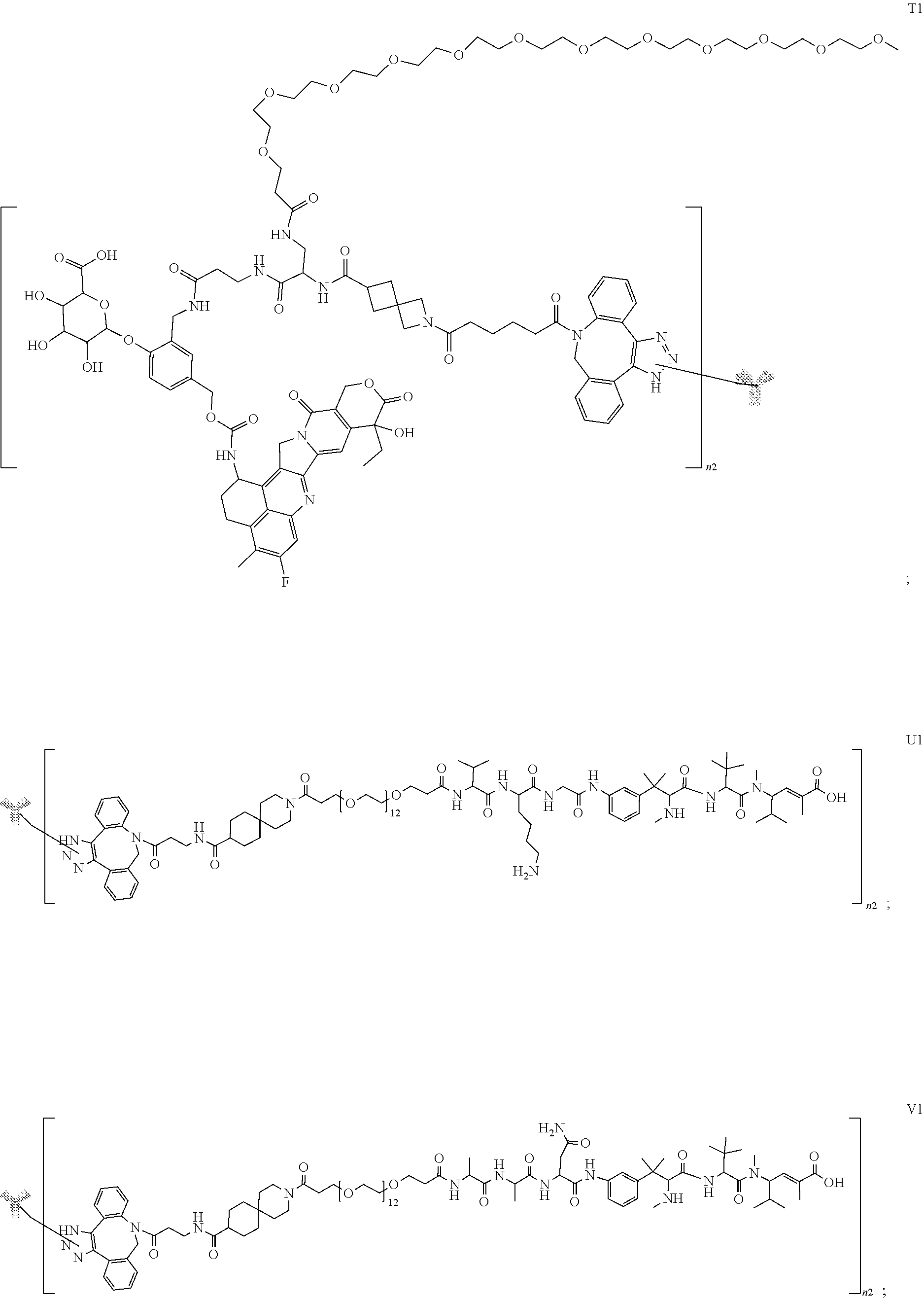

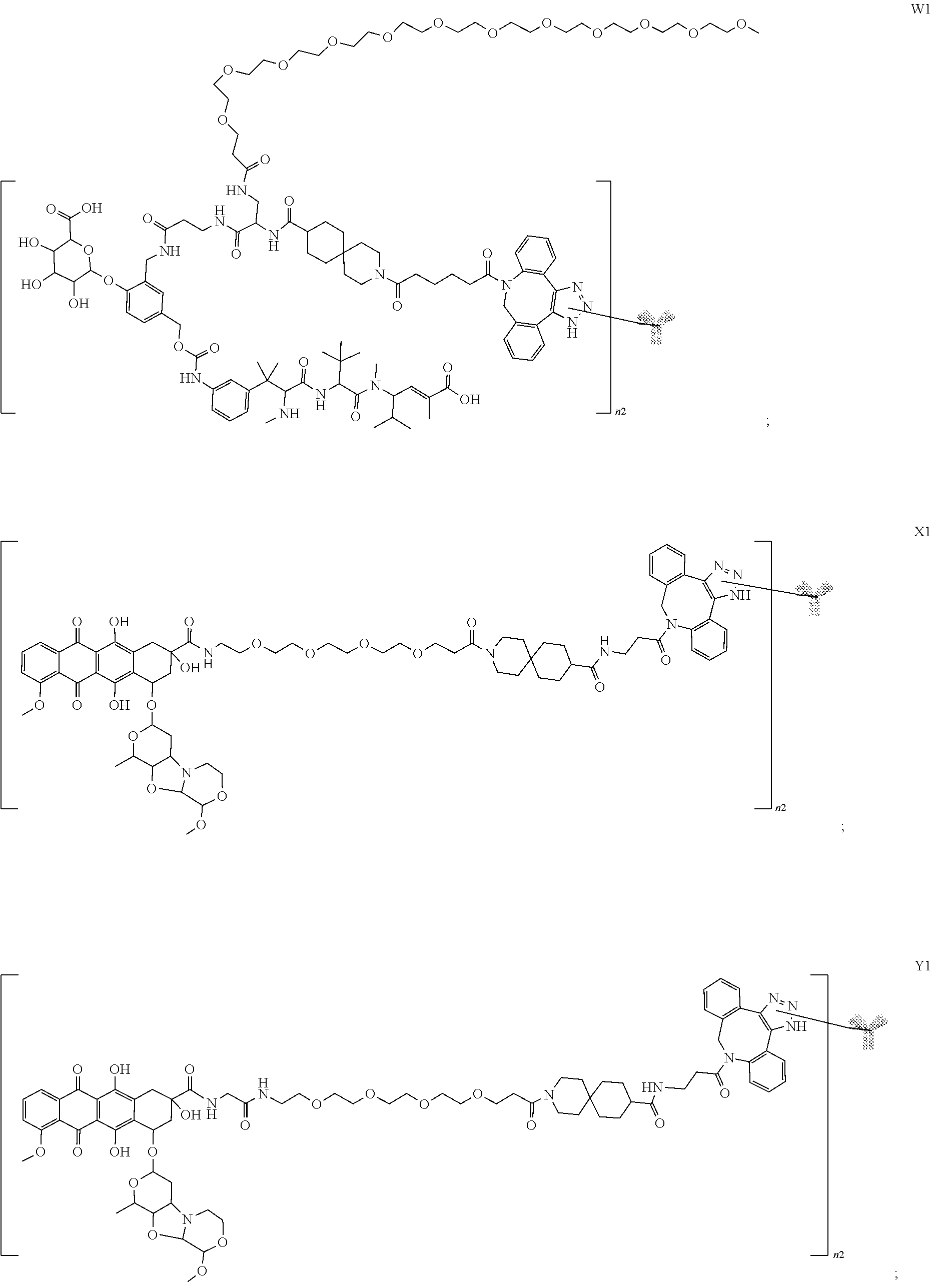

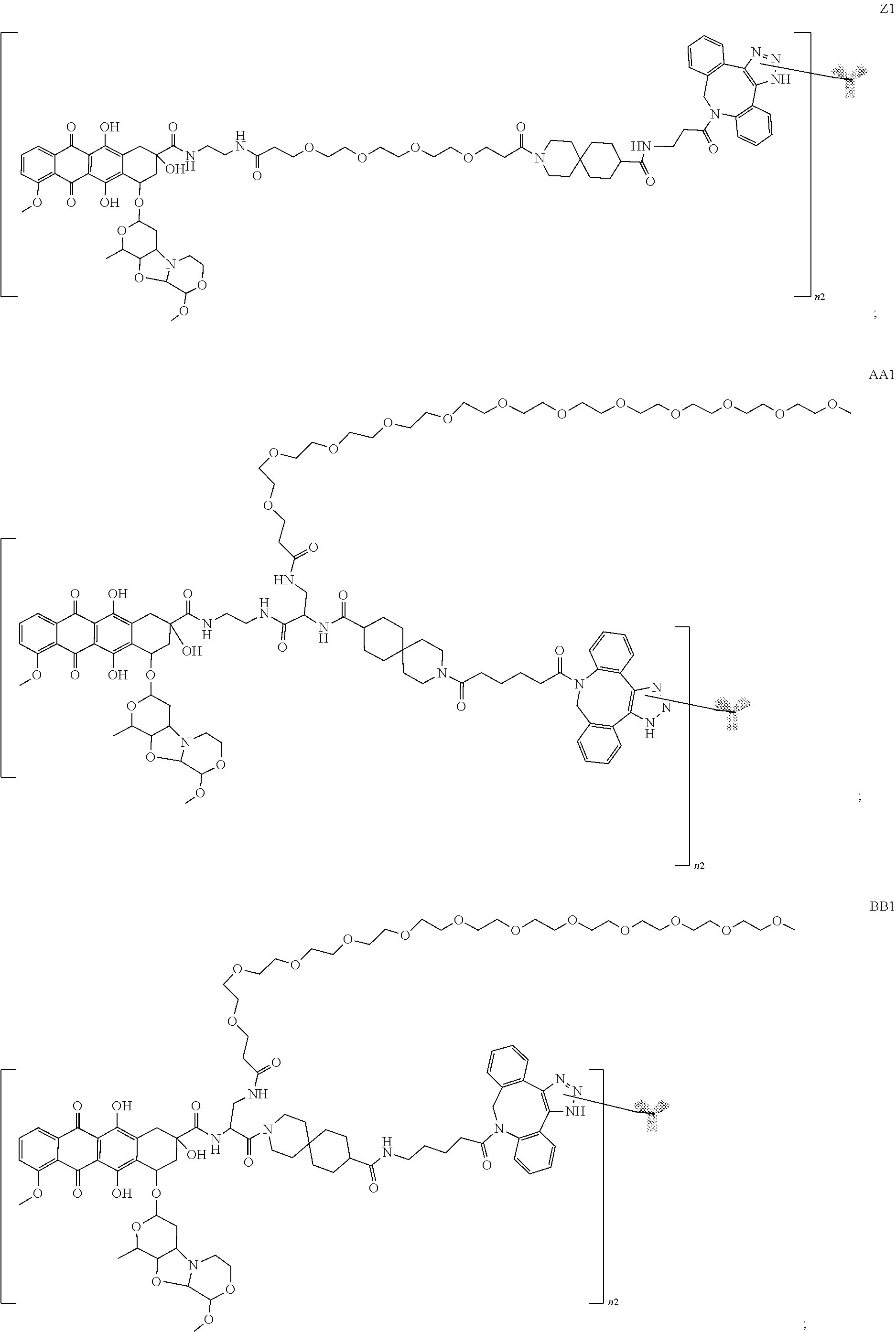

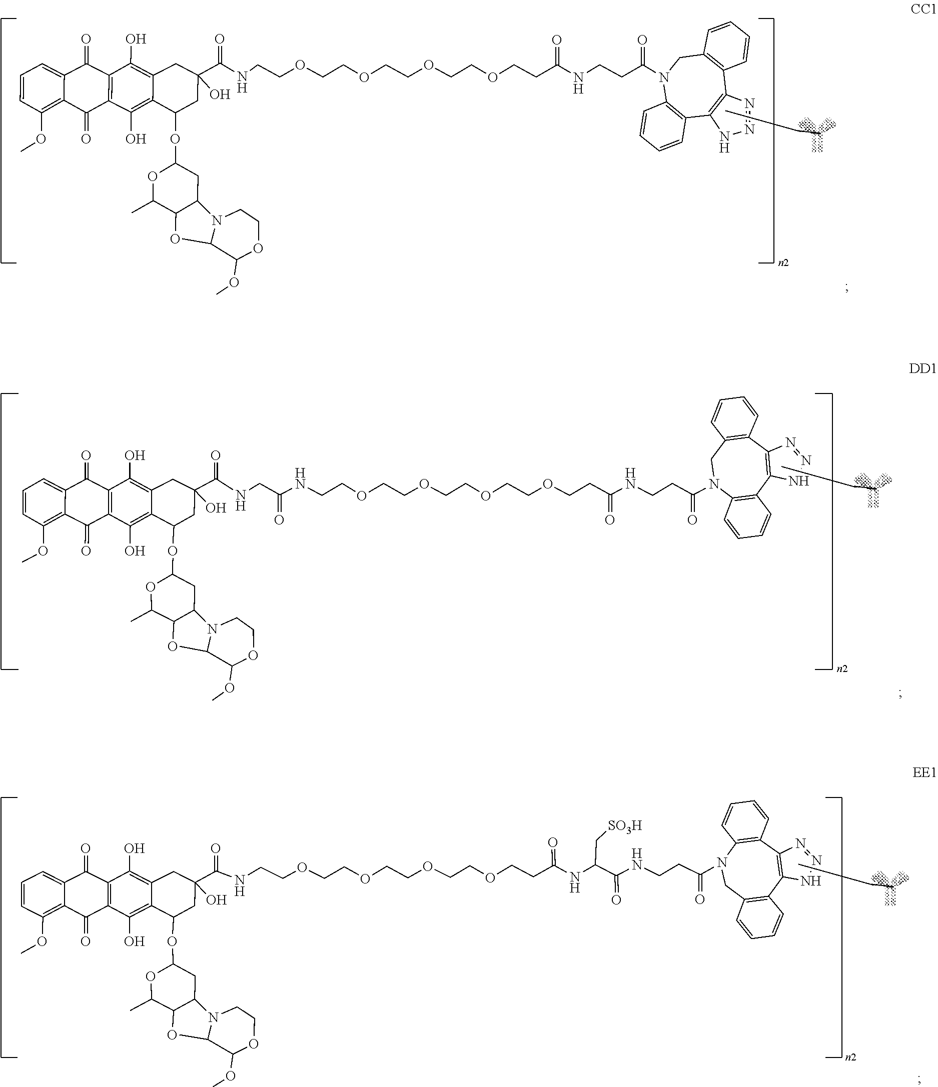

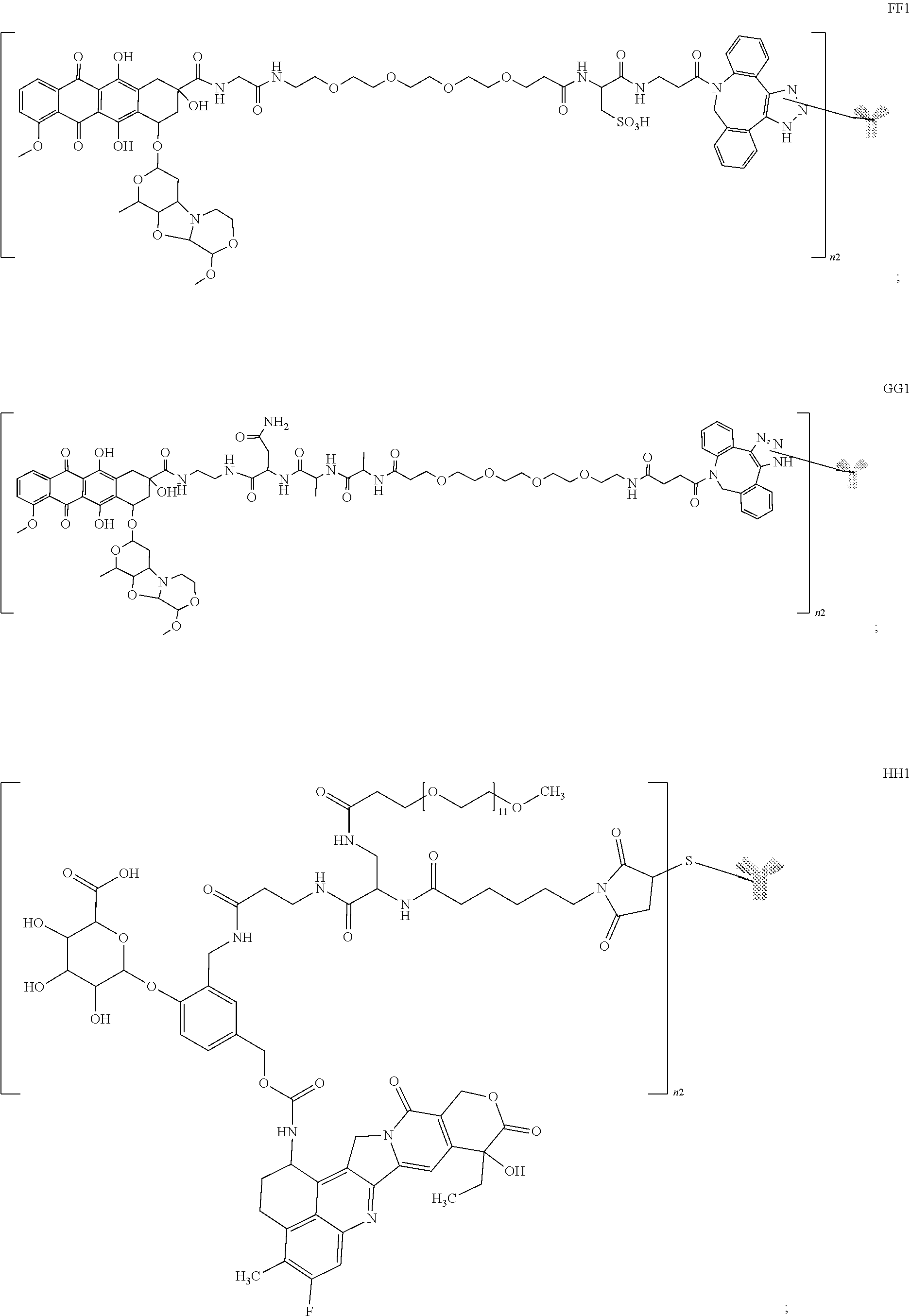

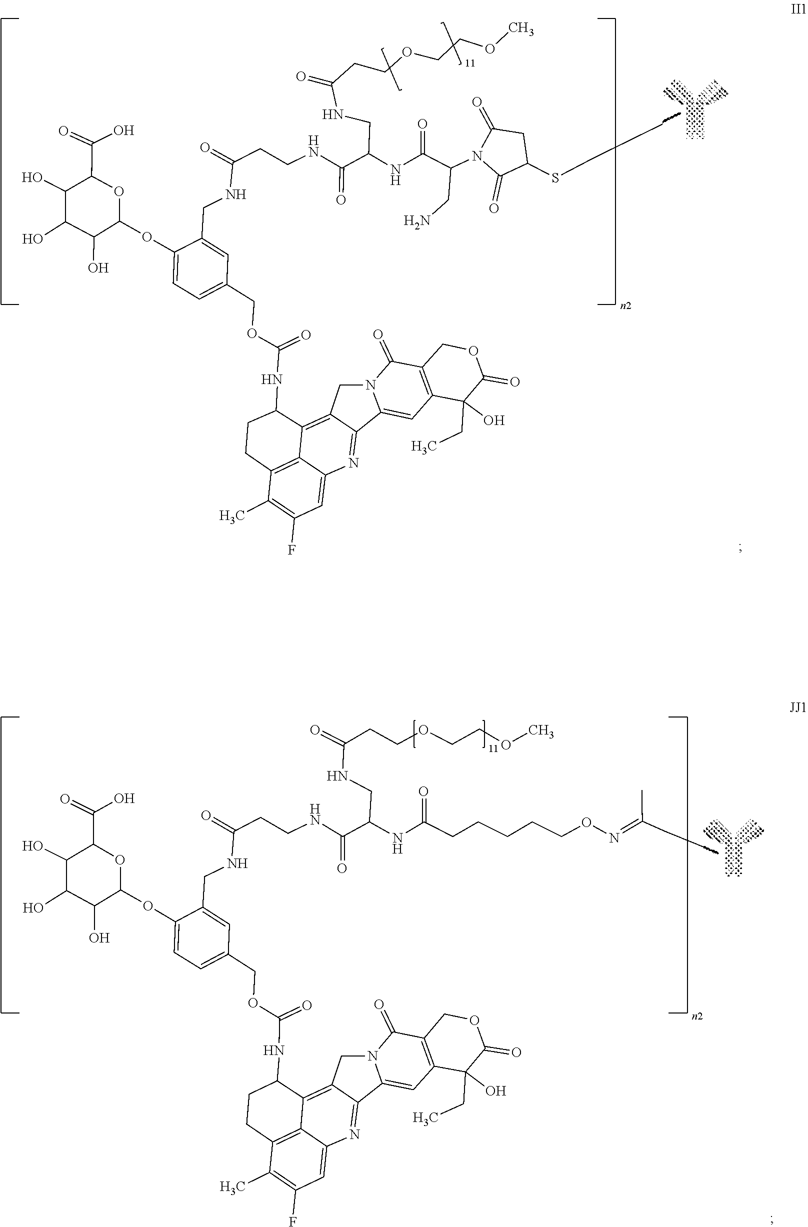

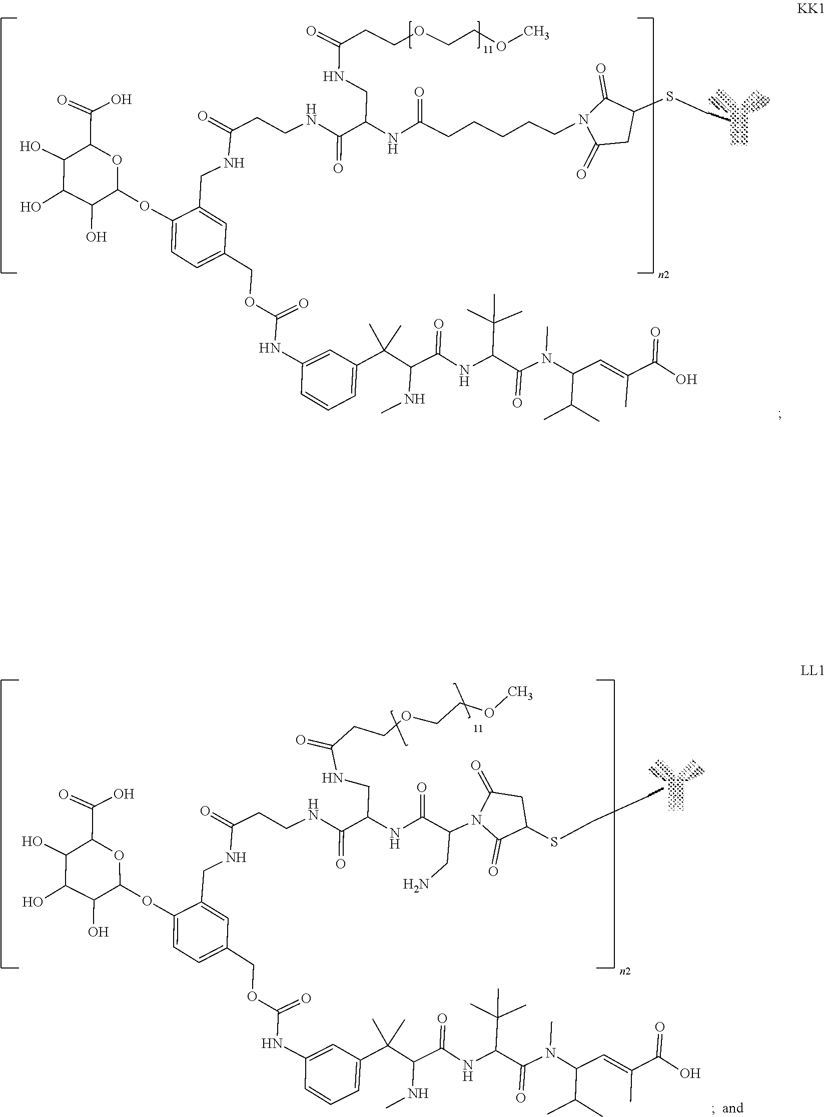

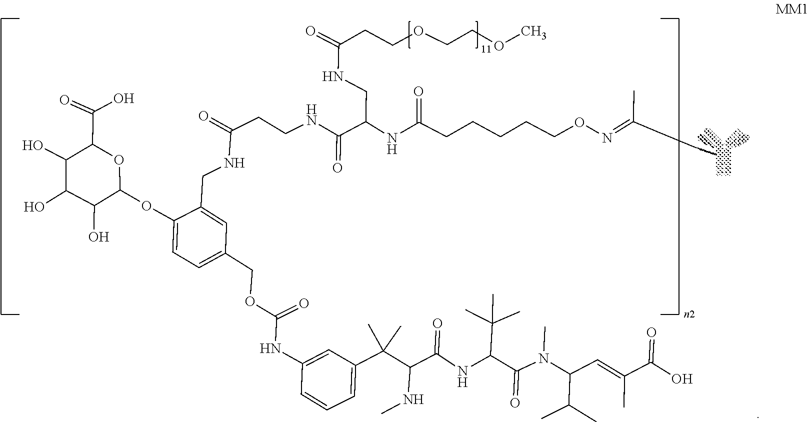

In any of the foregoing embodiments wherein the anti-ROR1 conjugate has a structure according to any one of Conjugates A-GG, the bracketed structure can be covalently bonded to one or more non-natural amino acids of the antibody, wherein the one or more non-natural amino acids are located at sites selected from the group consisting of: HC-F404, HC-Y180, HC-F241, LC-K42, and LC-E161, according to the Kabat or EU numbering scheme of Kabat. In some embodiments, the bracketed structure is covalently bonded to one or more non-natural amino acids at site HC-F404 of the antibody. In some embodiments, the bracketed structure is covalently bonded to one or more non-natural amino acids at site HC-Y180 of the antibody. In some embodiments, the bracketed structure is covalently bonded to one or more non-natural amino acids at site HC-F241 of the antibody. In some embodiments, the bracketed structure is covalently bonded to one or more non-natural amino acids at site LC-K42 of the antibody. In some embodiments, the bracketed structure is covalently bonded to one or more non-natural amino acids at site LC-E161 of the antibody. In some embodiments, the bracketed structures are covalently bonded to non-natural amino acids at sites HC-F404 and HC-Y180 of the antibody. In some embodiments, the bracketed structures are covalently bonded to non-natural amino acids at sites HC-F404, HC-Y180, and LC-K42 of the antibody. In some embodiments, the bracketed structures are covalently bonded to non-natural amino acids at sites HC-F404, HC-Y180, LC-K42, and LC-E161 of the antibody. In some embodiments, the bracketed structures are covalently bonded to non-natural amino acids at sites HC-F404, HC-Y180, and HC-F241 of the antibody. In some embodiments, the bracketed structures are covalently bonded to non-natural amino acids at sites HC-F404, HC-Y180, HC-F241, and LC-K42 of the antibody.

In particular embodiments, provided herein are anti-ROR1 conjugates according to any of the conjugates described herein wherein COMP indicates a residue of the non-natural amino acid according to Formula (30), below. In particular embodiments, provided herein are anti-ROR1 conjugates according to any of the conjugates described herein wherein COMP indicates a residue of the non-natural amino acid according to Formula (30), below, at heavy chain position 404 according to the EU numbering system. In particular embodiments, provided herein are anti-ROR1 conjugates according to any of the conjugates described herein wherein COMP indicates a residue of the non-natural amino acid according to Formula (30), below, at heavy chain position 180 according to the EU numbering system. In particular embodiments, provided herein are anti-ROR1 conjugates according to any of the conjugates described herein wherein COMP indicates a residue of the non-natural amino acid according to Formula (30), below, at heavy chain position 241 according to the EU numbering system. In particular embodiments, provided herein are anti-ROR1 conjugates according to any of the conjugates described herein wherein COMP indicates a residue of the non-natural amino acid according to Formula (30), below, at heavy chain position 222 according to the EU numbering system. In particular embodiments, provided herein are anti-ROR1 conjugates according to any of the conjugates described herein wherein COMP indicates a residue of the non-natural amino acid according to Formula (30), below, at light chain position 7 according to the Kabat or Chothia numbering system. In particular embodiments, provided herein are anti-ROR1 conjugates according to any of the conjugates described herein wherein COMP indicates a residue of the non-natural amino acid according to Formula (30), below, at light chain position 42 according to the Kabat or Chothia numbering system. In certain embodiments, PAY is selected from the group consisting of maytansine, hemiasterlin, amanitin, camptothecan, exatecan, exatecan derivative (DXd), SN-38, anthracycline, PNU-159682, PNU derivative (PNU-EDA), pyrrolobenzodiazepine (PBD), MMAF, and MMAE. In certain embodiments, PAY is maytansine. In certain embodiments, PAY is hemiasterlin. In certain embodiments, PAY is amanitin. In certain embodiments, PAY is exutecan. In certain embodiments, PAY is exatecan derivative Dxd. In certain embodiments, PAY is anthracycline. In certain embodiments, PAY is PNU-159682. In certain embodiments, PAY is PNU derivative (PNU-EDA). In certain embodiments, PAY is pyrrolobenzodiazepine. In certain embodiments, PAY is MMAF. In certain embodiments, PAY is MMAE.

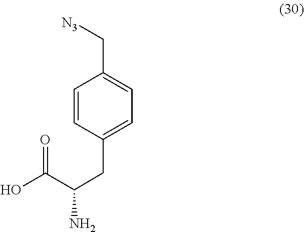 [SMILES: [N-]=[N+]=NCc1ccc(C[C@H](N)C(=O)O)cc1]

In particular embodiments, provided herein are anti-ROR1 conjugates according to any of the conjugates described herein wherein COMP indicates a residue of the non-natural amino acid according to Formula (56), below. In particular embodiments, provided herein are anti-ROR1 conjugates according to any of the conjugates described herein wherein COMP indicates a residue of the non-natural amino acid according to Formula (56), below, at heavy chain position 404 according to the EU numbering system. In particular embodiments, provided herein are anti-ROR1 conjugates according to any of the conjugates described herein wherein COMP indicates a residue of the non-natural amino acid according to Formula (56), below, at heavy chain position 180 according to the EU numbering system. In particular embodiments, provided herein are anti-ROR1 conjugates according to any of the conjugates described herein wherein COMP indicates a residue of the non-natural amino acid according to Formula (56), below, at heavy chain position 241 according to the EU numbering system. In particular embodiments, provided herein are anti-ROR1 conjugates according to any of the conjugates described herein wherein COMP indicates a residue of the non-natural amino acid according to Formula (56), below, at heavy chain position 222 according to the EU numbering system. In particular embodiments, provided herein are anti-ROR1 conjugates according to any of the conjugates described herein wherein COMP indicates a residue of the non-natural amino acid according to Formula (56), below, at light chain position 7 according to the Kabat or Chothia numbering system. In particular embodiments, provided herein are anti-ROR1 conjugates according to any of the conjugates described herein wherein COMP indicates a residue of the non-natural amino acid according to Formula (56), below, at light chain position 42 according to the Kabat or Chothia numbering system. In certain embodiments, PAY is selected from the group consisting of maytansine, hemiasterlin, amanitin, camptothecan, exatecan, exatecan derivative (DXd), SN-38, anthracycline, PNU-159682, PNU derivative (PNU-EDA), pyrrolobenzodiazepine (PBD), MMAF, and MMAE. In certain embodiments, PAY is maytansine. In certain embodiments, PAY is hemiasterlin. In certain embodiments, PAY is amanitin. In certain embodiments, PAY is exatecan. In certain embodiments, PAY is exatecan derivative (Dxd). In certain embodiments, PAY is deruxtecan. In certain embodiments, PAY is anthracycline. In certain embodiments, PAY is PNU-159682. In certain embodiments, PAY is PNU derivative (PNU-EDA). In certain embodiments, PAY is pyrrolobenzodiazepine. In certain embodiments, PAY is MMAF. In certain embodiments, PAY is MMAE.

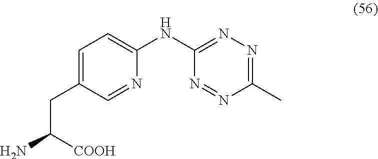 [SMILES: Cc1nnc(Nc2ccc(C[C@H](N)C(=O)O)cn2)nn1]

In particular embodiments, provided herein are anti-ROR1 conjugates according to any of the conjugates described herein wherein COMP indicates a non-natural amino acid residue of para-azido-L-phenylalanine. In particular embodiments, provided herein are anti-ROR1 conjugates according to any of the conjugates described herein wherein COMP indicates the non-natural amino acid residue para-azido-phenylalanine at heavy chain position 404 according to the EU numbering system. In particular embodiments, provided herein are anti-ROR1 conjugates according to any of the conjugates described herein wherein COMP indicates a non-natural amino acid residue of para-azido-L-phenylalanine at heavy chain position 180 according to the EU numbering system. In particular embodiments, provided herein are anti-ROR1 conjugates according to any of the conjugates described herein wherein COMP indicates a non-natural amino acid residue para-azido-L-phenylalanine at heavy chain position 241 according to the EU numbering system. In particular embodiments, provided herein are anti-ROR1 conjugates according to any of the conjugates described herein wherein COMP indicates a non-natural amino acid residue para-azido-L-phenylalanine at heavy chain position 222 according to the EU numbering system. In particular embodiments, provided herein are anti-ROR1 conjugates according to any of the conjugates described herein wherein COMP indicates a non-natural amino acid residue para-azido-L-phenylalanine at light chain position 7 according to the Kabat or Chothia numbering system. In particular embodiments, provided herein are anti-ROR1 conjugates according to any of the conjugates described herein wherein COMP indicates a non-natural amino acid residue para-azido-L-phenylalanine at light chain position 42 according to the Kabat or Chothia numbering system. In certain embodiments, PAY is selected from the group consisting of maytansine, hemiasterlin, amanitin, camptothecan, exatecan, exatecan derivative (DXd), SN-38, anthracycline, PNU-159682, pyrrolobenzodiazepine (PBD), MMAF, and MMAE. In certain embodiments, PAY is maytansine. In certain embodiments, PAY is hemiasterlin. In certain embodiments, PAY is amanitin. In certain embodiments, PAY is exatecan. In certain embodiments, PAY is exatecan derivative (Dxd). In certain embodiments, PAY is anthracycline. In certain embodiments, PAY is PNU-159682. In certain embodiments, PAY is PNU derivative (PNU-EDA). In certain embodiments, PAY is pyrrolobenzodiazepine. In certain embodiments, PAY is MMAF. In certain embodiments, PAY is MMAE.

### 3. Payloads

In addition to the payloads described above, the molecular payload can be any molecular entity that one of skill in the art might desire to conjugate to the polypeptide. In certain embodiments, the payload is a therapeutic moiety. In such embodiment, the antibody conjugate can be used to target the therapeutic moiety to its molecular target. In certain embodiments, the payload is a labeling moiety. In such embodiments, the antibody conjugate can be used to detect binding of the polypeptide to its target. In certain embodiments, the payload is a cytotoxic moiety. In such embodiments, the antibody conjugate can be used target the cytotoxic moiety to a diseased cell, for example a cancer cell, to initiate destruction or elimination of the cell. Conjugates comprising other molecular payloads apparent to those of skill in the art are within the scope of the conjugates described herein.

In certain embodiments, an antibody conjugate can have a payload selected from the group consisting of a label, a dye, a polymer, a water-soluble polymer, polyethylene glycol, a derivative of polyethylene glycol, a photocrosslinker, a cytotoxic compound, a radionuclide, a drug, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a peptide, a water-soluble dendrimer, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, or any combination thereof. In an embodiment, the payload is a label, a dye, a polymer, a cytotoxic compound, a radionuclide, a drug, an affinity label, a resin, a protein, a polypeptide, a polypeptide analog, an antibody, antibody fragment, a metal chelator, a cofactor, a fatty acid, a carbohydrate, a polynucleotide, a DNA, a RNA, a peptide, a fluorophore, or a carbon-linked sugar. In another embodiment, the payload is a label, a dye, a polymer, a drug, an antibody, antibody fragment, a DNA, an RNA, or a peptide.

Useful drug payloads include any cytotoxic or cytostatic agent. Useful classes of cytotoxic agents include, for example, antitubulin agents, auristatins, DNA minor groove binders, DNA replication inhibitors, alkylating agents (e.g., platinum complexes such as cis-platin, mono (platinum), bis (platinum) and tri-nuclear platinum complexes and carboplatin), anthracyclines, antibiotics, antifolates, antimetabolites, calmodulin inhibitors, chemotherapy sensitizers, duocarmycins, etoposides, fluorinated pyrimidines, ionophores, lexitropsins, maytansinoids, nitrosoureas, platinols, pore-forming compounds, purine antimetabolites, puromycins, radiation sensitizers, rapamycins, steroids, taxanes, topoisomerase inhibitors, vinca alkaloids, or the like.

Individual cytotoxic agents include, for example, an androgen, anthramycin (AMC), asparaginase, 5-azacytidine, azathioprine, bleomycin, busulfan, buthionine sulfoximine, calicheamicin, calicheamicin derivatives, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, DM1, DM4, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, rhizoxin, streptozotocin, tenoposide, 6-thioguanine, thioTEPA, topotecan, vinblastine, vincristine, vinorelbine, VP-16 and VM-26.

In some embodiments, suitable cytotoxic agents include, for example, DNA minor groove binders (e.g., enediynes and lexitropsins, a CBI compound; see also U.S. Pat. No. 6,130,237), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophycins, cemadotin, maytansinoids, discodermolide, eleutherobin, and mitoxantrone.

In some embodiments, the payload is an anti-tubulin agent. Examples of anti-tubulin agents include, but are not limited to, taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs, epothilones (e.g., epothilone A and B), nocodazole, colchicine and colcimid, estramustine, cryptophycins, cemadotin, maytansinoids, combretastatins, discodermolide, and eleutherobin.

In certain embodiments, the cytotoxic agent is a maytansinoid, another group of anti-tubulin agents. For example, in specific embodiments, the maytansinoid can be maytansine or DM1 (ImmunoGen, Inc.; see also Chari et al., 1992, Cancer Res. 52:127-131).

In some embodiments, the payload is an auristatin, such as auristatin E or a derivative thereof. For example, the auristatin E derivative can be an ester formed between auristatin E and a keto acid. For example, auristatin E can be reacted with paraacetyl benzoic acid or benzoylvaleric acid to produce AEB and AEVB, respectively. Other typical auristatin derivatives include AFP (auristatin phenylalanine phenylenediamine), MMAF (monomethyl auristatin F), and MMAE (monomethyl auristatin E). The synthesis and structure of auristatin derivatives are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In some embodiments, the payload is a hemiasterlin. Hemiasterlins suitable for use in the antibody-drug conjugates described herein are described, for example, in International Patent Publication No. WO 2016/2016/123582, which is incorporated herein by reference in its entirety.

In some embodiments, the payload is not a radioisotope. In some embodiments, the payload is not radioactive.

In some embodiments, the payload is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, the payload is tacrolimus, cyclosporine, FU506 or rapamycin. In further embodiments, the Drug is aldesleukin, alemtuzumab, alitretinoin, allopurinol, altretamine, amifostine, anastrozole, arsenic trioxide, bexarotene, bexarotene, calusterone, capecitabine, celecoxib, cladribine, Darbepoetin alfa, Denileukin diftitox, dexrazoxane, dromostanolone propionate, epirubicin, Epoetin alfa, estramustine, exemestane, Filgrastim, floxuridine, fludarabine, fulvestrant, gemcitabine, gemtuzumab ozogamicin (MYLOTARG), goserelin, idarubicin, ifosfamide, imatinib mesylate, Interferon alfa-2a, irinotecan, letrozole, leucovorin, levamisole, meclorethamine or nitrogen mustard, megestrol, mesna, methotrexate, methoxsalen, mitomycin C, mitotane, nandrolone phenpropionate, oprelvekin, oxaliplatin, pamidronate, pegademase, pegaspargase, pegfilgrastim, pentostatin, pipobroman, a pladienolide, plicamycin, porfimer sodium, procarbazine, quinacrine, rasburicase, Rituximab, Sargramostim, streptozocin, tamoxifen, temozolomide, teniposide, testolactone, thioguanine, toremifene, Tositumomab, Trastuzumab (HERCEPTIN), tretinoin, uracil mustard, valrubicin, vinblastine, vincristine, vinorelbine or zoledronate.

Other useful drug payloads include chemical compounds useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially uncialamycin, calicheamicin gammall, and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pladienolide B, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Other useful payloads include: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); (x) agents that act to regulate or inhibit activity of members of the poly(ADP-ribose) polymerase (PARP) family in tumors (e.g., Talazoparib (BMN-673), Iniparib (BSI 201), Veliparib (ABT-888), Olaparib (AZD-2281, trade name LYNPARZA™), Rucaparib (AG 014699), BGB-290, E7016, E7449, and CEP-9722); (xi) agents that act to regulate or inhibit activity of members of the histone deacetylase (HDAC) family in tumors (e.g., abexinostat, entinostat, gavinostat, 4SC-202, ACY-241, AR-42, CG200745, CHR-2845, CHR-3996, CXD101, MPTOE028, OBP-801, SHP-141, CUDC-101, KA2507, panobinostat, pracinostat, quisinostat, resminostat, ricolinostat); (xii) agents that act to regulate or inhibit activity of mitochondrial enzyme isocitrate dehydrogenase type 2 (IDH2) in tumors (e.g., enasidenib mesylate (CC-90007, AG-221 mesylate); and (xiii) pharmaceutically acceptable salts, acids and derivatives of any of the above. Other useful payloads include anti-angiogenic agents, including, e.g., MMP-2 (matrix-metalloproteinase 2) inhibitors, MMP-9 (matrix-metalloproteinase 9) inhibitors, COX-II (cyclooxygenase II) inhibitors, and VEGF receptor tyrosine kinase inhibitors. Examples of such useful matrix metalloproteinase inhibitors that can be used in combination with the present compounds/compositions are described in WO 96/33172, WO 96/27583, EP 818442, EP 1004578, WO 98/07697, WO 98/03516, WO 98/34918, WO 98/34915, WO 98/33768, WO 98/30566, EP 606,046, EP 931,788, WO 90/05719, WO 99/52910, WO 99/52889, WO 99/29667, WO 99/07675, EP 945864, U.S. Pat. Nos. 5,863,949, 5,861,510, and EP 780,386, all of which are incorporated herein in their entireties by reference. Examples of VEGF receptor tyrosine kinase inhibitors include 4-(4-bromo-2-fluoroanilino)-6-methoxy-7-(1-methylpiperidin-4-ylmethoxy) quinazoline (ZD6474; Example 2 within WO 01/32651), 4-(4-fluoro-2-methylindol-5-yloxy)-6-methoxy-7-(3-pyrrolidin-1-ylpropoxy) quinazoline (AZD2171; Example 240 within WO 00/47212), vatalanib (PTK787; WO 98/35985) and SU11248 (sunitinib; WO 01/60814), and compounds such as those disclosed in PCT Publication Nos. WO 97/22596, WO 97/30035, WO 97/32856, and WO 98/13354).

In certain embodiments, the payload is selected from the group consisting of maytansine, hemiasterlin, amanitin, exatecan, deruxtecan (DXd), anthracycline, PNU-159682, pyrrolobenzodiazepine (PBD), MMAF, and MMAE. In certain embodiments, the the payload is maytansine. In certain embodiments, the payload is hemiasterlin. In certain embodiments, the payload is amanitin. In certain embodiments, the payload is hemiasterlin. In certain embodiments, the payload is amanitin. In certain embodiments, the payload is hemiasterlin. In certain embodiments, the payload is deruxtecan. In certain embodiments, the payload is hemiasterlin. In certain embodiments, the payload is anthracycline. In certain embodiments, the payload is hemiasterlin. In certain embodiments, the payload is PNU-159682. In certain embodiments, the payload is hemiasterlin. In certain embodiments, the payload is pyrrolobenzodiazepine. In certain embodiments, the payload is MMAF. In certain embodiments, the payload is MMAE.

In certain embodiments, the payload is an antibody or an antibody fragment. In certain embodiments, the payload antibody or fragment can be encoded by any of the immunoglobulin genes recognized by those of skill in the art. The immunoglobulin genes include, but are not limited to, the κ, λ, α, γ (IgG1, IgG2, IgG3, and IgG4), δ, ε and μ constant region genes, as well as the immunoglobulin variable region genes. The term includes full-length antibody and antibody fragments recognized by those of skill in the art, and variants thereof. Exemplary fragments include but are not limited to Fv, Fc, Fab, and (Fab′)₂, single chain Fv (scFv), diabodies, triabodies, tetrabodies, bifunctional hybrid polypeptides, CDR1, CDR2, CDR3, combinations of CDR's, variable regions, framework regions, constant regions, and the like.

In certain embodiments, the payload is one or more water-soluble polymers. A wide variety of macromolecular polymers and other molecules can be linked to the polypeptides described herein to modulate biological properties of the polypeptide, and/or provide new biological properties to the polypeptide. These macromolecular polymers can be linked to the polypeptide via a naturally encoded amino acid, via a non-naturally encoded amino acid, or any functional substituent of a natural or modified amino acid, or any substituent or functional group added to a natural or modified amino acid. The molecular weight of the polymer may be of a wide range, including but not limited to, between about 100 Da and about 100,000 Da or more.

The polymer selected may be water soluble so that a protein to which it is attached does not precipitate in an aqueous environment, such as a physiological environment. The polymer may be branched or unbranched. Preferably, for therapeutic use of the end-product preparation, the polymer will be pharmaceutically acceptable.

In certain embodiments, the proportion of polyethylene glycol molecules to polypeptide molecules will vary, as will their concentrations in the reaction mixture. In general, the optimum ratio (in terms of efficiency of reaction in that there is minimal excess unreacted protein or polymer) may be determined by the molecular weight of the polyethylene glycol selected and on the number of available reactive groups available. As relates to molecular weight, typically the higher the molecular weight of the polymer, the fewer number of polymer molecules which may be attached to the protein. Similarly, branching of the polymer should be taken into account when optimizing these parameters. Generally, the higher the molecular weight (or the more branches) the higher the polymer: protein ratio.

The water-soluble polymer may be any structural form including but not limited to linear, forked or branched. Typically, the water soluble polymer is a poly(alkylene glycol), such as poly(ethylene glycol) (PEG), but other water soluble polymers can also be employed. By way of example, PEG is used to describe certain embodiments.

PEG is a well-known, water-soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented as linked to a polypeptide by the formula: XO—(CH₂CH₂O)ₙ—CH₂CH₂—Y where n is 2 to 10,000, X is H or a terminal modification, including but not limited to, a C₁₋₄ alkyl, and Y is the attachment point to the polypeptide.

In some cases, a PEG terminates on one end with hydroxy or methoxy, i.e., X is H or CH₃ (“methoxy PEG”). Alternatively, the PEG can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester, and aldehydes) as well as functional groups that are inert to the 20 common amino acids but that react specifically with complementary functional groups present in non-naturally encoded amino acids (including but not limited to, azide groups, alkyne groups). It is noted that the other end of the PEG, which is shown in the above formula by Y, will attach either directly or indirectly to a polypeptide via a naturally-occurring or non-naturally encoded amino acid. For instance, Y may be an amide, carbamate, or urea linkage to an amine group (including but not limited to, the epsilon amine of lysine or the N-terminus) of the polypeptide. Alternatively, Y may be a maleimide linkage to a thiol group (including but not limited to, the thiol group of cysteine). Alternatively, Y may be a linkage to a residue not commonly accessible via the 20 common amino acids. For example, an azide group on the PEG can be reacted with an alkyne group on the polypeptide to form a Huisgen [3+2] cycloaddition product. Alternatively, an alkyne group on the PEG can be reacted with an azide group present in a non-naturally encoded amino acid, such as the modified amino acids described herein, to form a similar product. In some embodiments, a strong nucleophile (including but not limited to, hydrazine, hydrazide, hydroxylamine, semicarbazide) can be reacted with an aldehyde or ketone group present in a non-naturally encoded amino acid to form a hydrazone, oxime or semicarbazone, as applicable, which in some cases can be further reduced by treatment with an appropriate reducing agent. Alternatively, the strong nucleophile can be incorporated into the polypeptide via a non-naturally encoded amino acid and used to react preferentially with a ketone or aldehyde group present in the water-soluble polymer.

Any molecular mass for a PEG can be used as practically desired, including but not limited to, from about 100 Daltons (Da) to 100,000 Da or more as desired (including but not limited to, sometimes 0.1-50 kDa or 10-40 kDa). Branched chain PEGs, including but not limited to, PEG molecules with each chain having a MW ranging from 1-100 kDa (including but not limited to, 1-50 kDa or 5-20 kDa) can also be used. A wide range of PEG molecules are described in, including but not limited to, the Shearwater Polymers, Inc. catalog, and the Nektar Therapeutics catalog, incorporated herein by reference.

Generally, at least one terminus of the PEG molecule is available for reaction with the antibody. For example, PEG derivatives bearing alkyne and azide moieties for reaction with amino acid side chains can be used to attach PEG to non-naturally encoded amino acids as described herein. If the non-naturally encoded amino acid comprises an azide, then the PEG will typically contain either an alkyne moiety to effect formation of the [3+2] cycloaddition product or an activated PEG species (i.e., ester, carbonate) containing a phosphine group to effect formation of the amide linkage. Alternatively, if the non-naturally encoded amino acid comprises an alkyne, then the PEG will typically contain an azide moiety to effect formation of the [3+2] Huisgen cycloaddition product. If the non-naturally encoded amino acid comprises a carbonyl group, the PEG will typically comprise a potent nucleophile (including but not limited to, a hydrazide, hydrazine, hydroxylamine, or semicarbazide functionality) in order to effect formation of corresponding hydrazone, oxime, and semicarbazone linkages, respectively. In other alternatives, a reverse of the orientation of the reactive groups described herein can be used, i.e., an azide moiety in the non-naturally encoded amino acid can be reacted with a PEG derivative containing an alkyne.

In some embodiments, the polypeptide variant with a PEG derivative contains a chemical functionality that is reactive with the chemical functionality present on the side chain of the non-naturally encoded amino acid.

In certain embodiments, the payload is an azide- or acetylene-containing polymer comprising a water-soluble polymer backbone having an average molecular weight from about 800 Da to about 100,000 Da. The polymer backbone of the water-soluble polymer can be poly(ethylene glycol). However, it should be understood that a wide variety of water soluble polymers including but not limited to poly(ethylene)glycol and other related polymers, including poly(dextran) and poly(propylene glycol), are also suitable for use and that the use of the term PEG or poly(ethylene glycol) is intended to encompass and include all such molecules. The term PEG includes, but is not limited to, poly(ethylene glycol) in any of its forms, including bifunctional PEG, multiarmed PEG, derivatized PEG, forked PEG, branched PEG, pendent PEG (i.e. PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, glycerol oligomers, pentaerythritol and sorbitol. The central branch moiety can also be derived from several amino acids, such as lysine. The branched poly(ethylene glycol) can be represented in general form as R(-PEG-OH)ₘ in which R is derived from a core moiety, such as glycerol, glycerol oligomers, or pentaerythritol, and m represents the number of arms. Multi-armed PEG molecules, such as those described in U.S. Pat. Nos. 5,932,462 5,643,575; 5,229,490; 4,289,872; U.S. Pat. Appl. 2003/0143596; WO 96/21469; and WO 93/21259, each of which is incorporated by reference herein in its entirety, can also be used as the polymer backbone.

Branched PEG can also be in the form of a forked PEG represented by PEG(-YCHZ₂)ₙ, where Y is a linking group and Z is an activated terminal group linked to CH by a chain of atoms of defined length.

Yet another branched form, the pendant PEG, has reactive groups, such as carboxyl, along the PEG backbone rather than at the end of PEG chains.

In addition to these forms of PEG, the polymer can also be prepared with weak or degradable linkages in the backbone. For example, PEG can be prepared with ester linkages in the polymer backbone that are subject to hydrolysis. As shown herein, this hydrolysis results in cleavage of the polymer into fragments of lower molecular weight: -PEG-CO₂-PEG-+H₂O→PEG-CO₂H+HO-PEG- It is understood by those skilled in the art that the term poly(ethylene glycol) or PEG represents or includes all the forms known in the art including but not limited to those disclosed herein.

Many other polymers are also suitable for use. In some embodiments, polymer backbones that are water-soluble, with from 2 to about 300 termini, are particularly suitable. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da.

Those of ordinary skill in the art will recognize that the foregoing list for substantially water-soluble backbones is by no means exhaustive and is merely illustrative, and that all polymeric materials having the qualities described herein are contemplated as being suitable for use.

In some embodiments the polymer derivatives are “multi-functional”, meaning that the polymer backbone has at least two termini, and possibly as many as about 300 termini, functionalized or activated with a functional group. Multifunctional polymer derivatives include, but are not limited to, linear polymers having two termini, each terminus being bonded to a functional group which may be the same or different.

### 4. Linkers

In certain embodiments, the antibodies can be linked to the payloads with one or more linkers capable of reacting with an antibody amino acid and with a payload group. The one or more linkers can be any linkers apparent to those of skill in the art.

The term “linker” is used herein to refer to groups or bonds that normally are formed as the result of a chemical reaction and typically are covalent linkages.

Useful linkers include those described herein. In certain embodiments, the linker is any divalent or multivalent linker known to those of skill in the art. Useful divalent linkers include alkylene, substituted alkylene, heteroalkylene, substituted heteroalkylene, arylene, substituted arylene, heteroarlyene, and substituted heteroarylene. In certain embodiments, the linker is C₁₋₁₀ alkylene or C₁₋₁₀ heteroalkylene. In some embodiments, the C₁₋₁₀heteoalkylene is PEG.

In certain embodiments, the linker is hydrolytically stable. Hydrolytically stable linkages means that the linkages are substantially stable in water and do not react with water at useful pH values, including but not limited to, under physiological conditions for an extended period of time, perhaps even indefinitely. In certain embodiments, the linker is hydrolytically unstable. Hydrolytically unstable or degradable linkages mean that the linkages are degradable in water or in aqueous solutions, including for example, blood. Enzymatically unstable or degradable linkages mean that the linkage can be degraded by one or more enzymes.

As understood in the art, PEG and related polymers may include degradable linkages in the polymer backbone or in the linker group between the polymer backbone and one or more of the terminal functional groups of the polymer molecule. For example, ester linkages formed by the reaction of PEG carboxylic acids or activated PEG carboxylic acids with alcohol groups on a biologically active agent generally hydrolyze under physiological conditions to release the agent.

Other hydrolytically degradable linkages include, but are not limited to, carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

A number of different cleavable linkers are known to those of skill in the art. See U.S. Pat. Nos. 4,618,492; 4,542,225, and 4,625,014. The mechanisms for release of an agent from these linker groups include, for example, irradiation of a photolabile bond and acid-catalyzed hydrolysis. U.S. Pat. No. 4,671,958, for example, includes a description of immunoconjugates comprising linkers which are cleaved at the target site in vivo by the proteolytic enzymes of the patient's complement system. The length of the linker may be predetermined or selected depending upon a desired spatial relationship between the polypeptide and the molecule linked to it. In view of the large number of methods that have been reported for attaching a variety of radiodiagnostic compounds, radiotherapeutic compounds, drugs, toxins, and other agents to polypeptides one skilled in the art will be able to determine a suitable method for attaching a given agent to a polypeptide.

The linker may have a wide range of molecular weight or molecular length. Larger or smaller molecular weight linkers may be used to provide a desired spatial relationship or conformation between the polypeptide and the linked entity. Linkers having longer or shorter molecular length may also be used to provide a desired space or flexibility between the polypeptide and the linked entity. Similarly, a linker having a particular shape or conformation may be utilized to impart a particular shape or conformation to the polypeptide or the linked entity, either before or after the polypeptide reaches its target. The functional groups present on each end of the linker may be selected to modulate the release of a polypeptide or a payload under desired conditions. This optimization of the spatial relationship between the polypeptide and the linked entity may provide new, modulated, or desired properties to the molecule.

In some embodiments, provided herein are water-soluble bifunctional linkers that have a dumbbell structure that includes: a) an azide, an alkyne, a hydrazine, a hydrazide, a hydroxylamine, or a carbonyl-containing moiety on at least a first end of a polymer backbone; and b) at least a second functional group on a second end of the polymer backbone. The second functional group can be the same or different as the first functional group. The second functional group, in some embodiments, is not reactive with the first functional group. In some embodiments, water-soluble compounds that comprise at least one arm of a branched molecular structure are provided. For example, the branched molecular structure can be a dendritic structure.

In some embodiments, the linker is derived from a linker precursor selected from the group consisting of: N-succinimidyl-3-(2-pyridyldithio) propionate (SPDP), N-succinimidyl 4-(2-pyridyldithio) pentanoate (SPP), N-succinimidyl 4-(2-pyridyldithio) butanoate (SPDB), N-succinimidyl-4-(2-pyridyldithio)-2-sulfo-butanoate (sulfo-SPDB), N-succinimidyl iodoacetate (SIA), N-succinimidyl (4-iodoacetyl)aminobenzoate (SIAB), maleimide PEG NHS, N-succinimidyl 4-(maleimidomethyl)cyclohexanecarboxylate (SMCC), N-sulfosuccinimidyl 4-(maleimidomethyl)cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1). In a specific embodiment, the linker is derived from the linker precursor N-succinimidyl 4-(maleimidomethyl)cyclohexanecarboxylate (SMCC).

In some embodiments, the linker is derived from a linker precursor selected from the group consisting of dipeptides, tripeptides, tetrapeptides, and pentapeptides. In such embodiments, the linker can be cleaved by a protease. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit), glycine-glycine-glycine (gly-gly-gly), and glycine-methoxyethoxyethyl) serine-valine (gly-val-citalanine OMESerValAla).

In some embodiments, a linker comprises a self-immolative spacer. In certain embodiments, the self-immolative spacer comprises p-aminobenzyl. In some embodiments, a p-aminobenzyl alcohol is attached to an amino acid unit via an amide bond, and a carbamate, methylcarbamate, or carbonate is made between the benzyl alcohol and the payload (Hamann et al. (2005) Expert Opin. Ther. Patents (2005) 15:1087-1103). In some embodiments, the linker comprises p-aminobenzyloxycarbonyl (PAB). Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al. (1995) Chemistry Biology 2:223), appropriately substituted bicyclo[2.2.1] and bicyclo[2.2.2] ring systems (Storm et al. (1972) J. Amer. Chem. Soc. 94:5815) and 2-aminophenylpropionic acid amides (Amsberry, et al. (1990) J. Org. Chem. 55:5867). Linkage of a drug to the α-carbon of a glycine residue is another example of a self-immolative spacer that may be useful in conjugates (Kingsbury et al. (1984) J. Med. Chem. 27:1447).

In certain embodiments, linker precursors can be combined to form larger linkers. For instance, in certain embodiments, linkers comprise the dipeptide valine-citrulline and p-aminobenzyloxycarbonyl. These are also referenced as citValCit-PAB linkers.

In certain embodiments, the payloads can be linked to the linkers, referred to herein as a linker-payload, with one or more linker groups capable of reacting with an antibody amino acid group. The one or more linkers can be any linkers apparent to those of skill in the art or those set forth herein.

Additional linkers are disclosed herein, such as, for example, the linker precursors (A)-(L) described below.

### 5. Antibodies and Antibody Specificity

Provided herein are antibodies that selectively bind human ROR1. In some aspects, the antibody selectively binds to the extracellular domain of human ROR1 (human ROR1).

Also provided herein are conjugates that comprise antibodies that selectively bind human receptor tyrosine kinase orphan receptor 1. In some aspects, the antibody of the conjugate selectively binds to the extracellular domain of human receptor tyrosine kinase orphan receptor 1 (human ROR1).

In some embodiments, the antibody binds to a homolog of human ROR1. In some aspects, the antibody binds to a homolog of human ROR1 from a species selected from monkeys, mice, dogs, cats, rats, cows, horses, goats and sheep. In some aspects, the homolog is a cynomolgus monkey homolog. In some aspects, the homolog is a mouse or murine analog.

In some embodiments, the antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 7, 8, 9, 10, 11, 12, 13, 14, 15, or 16 residues in length.

In some aspects, the Kabat/Chothia CDR-L1 of the antibody is 10, 11, 12, 13, 14, 15, or 16 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the antibody comprises a light chain. In some aspects, the light chain is a kappa light chain. In some aspects, the light chain is a lambda light chain.

In some embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

In some embodiments, the antibody is an affinity matured antibody. In some aspects, the antibody is an affinity matured antibody derived from an illustrative sequence provided in this disclosure.

The antibodies provided herein may be useful for the treatment of a variety of diseases and conditions including cancers. In some embodiments, the antibodies provided herein may be useful for the treatment of cancers of solid tumors. For example, the antibodies provided herein can be useful for the treatment of colorectal cancer.

### 5.1 CDR-H3 Sequences

In some embodiments, the antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a V_{H} sequence provided in SEQ ID NOs: 854-1020.

In some embodiments, the antibody comprises a CDR-H3 sequence comprising, consisting of, or consisting essentially of a sequence selected from SEQ ID NOs: 672-838. In some aspects, the antibody comprises a CDR-H3 sequence comprising, consisting of, or consisting essentially of SEQ ID NO:672. In some aspects, the antibody comprises a CDR-H3 sequence comprising, consisting of, or consisting essentially of SEQ ID NO:673. In some aspects, the antibody comprises a CDR-H3 sequence comprising, consisting of, or consisting essentially of SEQ ID NO:674. In some aspects, the antibody comprises a CDR-H3 sequence comprising, consisting of, or consisting essentially of SEQ ID NO:675. In some aspects, the antibody comprises a CDR-H3 sequence comprising, consisting of, or consisting essentially of SEQ ID NO:676. In some aspects, the antibody comprises a CDR-H3 sequence comprising, consisting of, or consisting essentially of SEQ ID NO:677. In some aspects, the antibody comprises a CDR-H3 sequence comprising, consisting of, or consisting essentially of SEQ ID NO:678. In some aspects, the antibody comprises a CDR-H3 sequence comprising, consisting of, or consisting essentially of SEQ ID NO:679. In some aspects, the antibody comprises a CDR-H3 sequence comprising, consisting of, or consisting essentially of SEQ ID NO:680. In some aspects, the antibody comprises a CDR-H3 sequence comprising, consisting of, or consisting essentially of SEQ ID NO:681. In some aspects, the antibody comprises a CDR-H3 sequence comprising, consisting of, or consisting essentially of SEQ ID NO:682. In some aspects, the antibody comprises a CDR-H3 sequence comprising, consisting of, or consisting essentially of SEQ ID NO:683. In some aspects, the antibody comprises a CDR-H3 sequence comprising, consisting of, or consisting essentially of SEQ ID NO:684. In some aspects, the antibody comprises a CDR-H3 sequence comprising, consisting of, or consisting essentially of SEQ ID NO:685. In some aspects, the antibody comprises a CDR-H3 sequence comprising, consisting of, or consisting essentially of SEQ ID NO:686. In some aspects, the antibody comprises a CDR-H3 sequence comprising, consisting of, or consisting essentially of SEQ ID NO:687. In some aspects, the antibody comprises a CDR-H3 sequence comprising, consisting of, or consisting essentially of SEQ ID NO:688. In some aspects, the antibody comprises a CDR-H3 sequence comprising, consisting of, or consisting essentially of SEQ ID NO:689. In some aspects, the antibody comprises a CDR-H3 sequence comprising, consisting of, or consisting essentially of SEQ ID NO:690. In some aspects, the antibody comprises a CDR-H3 sequence comprising, consisting of, or consisting essentially of SEQ ID NO:691. In some aspects, the antibody comprises a CDR-H3 sequence comprising, consisting of, or consisting essentially of SEQ ID NO:692. In some aspects, the antibody comprises a CDR-H3 sequence comprising, consisting of, or consisting essentially of SEQ ID NO:693. In some aspects, the antibody comprises a CDR-H3 sequence comprising, consisting of, or consisting essentially of SEQ ID NO:694. In some aspects, the antibody comprises a CDR-H3 sequence comprising, consisting of, or consisting essentially of SEQ ID NO:695. In some aspects, the antibody comprises a CDR-H3 sequence comprising, consisting of, or consisting essentially of SEQ ID NO:696. In some aspects, the antibody comprises a CDR-H3 sequence comprising, consisting of, or consisting essentially of SEQ ID NO:697. In some aspects, the antibody comprises a CDR-H3 sequence comprising, consisting of, or consisting essentially of SEQ ID NO:698. In some aspects, the antibody comprises a CDR-H3 sequence comprising, consisting of, or consisting essentially of SEQ ID NO:699.

In some aspects, the antibody comprises a CDR-H3 sequence comprising, consisting of, or consisting essentially of SEQ ID NO:700. In some aspects, the antibody comprises a CDR-H3 sequence comprising, consisting of, or consisting essentially of SEQ ID NO: 701. In some aspects, the antibody comprises a CDR-H3 sequence comprising, consisting of, or consisting essentially of SEQ ID NO:702. In some aspects, the antibody comprises a CDR-H3 sequence comprising, consisting of, or consisting essentially of SEQ ID NO: 703. In some aspects, the antibody comprises a CDR-H3 sequence comprising, consisting of, or consisting essentially of SEQ ID NO:704. In some aspects, the antibody comprises a CDR-H3 sequence comprising, consisting of, or consisting essentially of SEQ ID NO: 705. In some aspects, the antibody comprises a CDR-H3 sequence comprising, consisting of, or consisting essentially of SEQ ID NO:706. In some aspects, the antibody comprises a CDR-H3 sequence comprising, consisting of, or consisting essentially of SEQ ID NO: 707. In some aspects, the antibody comprises a CDR-H3 sequence comprising, consisting of, or consisting essentially of SEQ ID NO:708. In some aspects, the antibody comprises a CDR-H3 sequence comprising, consisting of, or consisting essentially of SEQ ID NO: 709. In some aspects, the antibody comprises a CDR-H3 sequence comprising, consisting of, or consisting essentially of SEQ ID NO:710. In some aspects, the antibody comprises a CDR-H3 sequence comprising, consisting of, or consisting essentially of SEQ ID NO: 711. In some aspects, the antibody comprises a CDR-H3 sequence comprising, consisting of, or consisting essentially of SEQ ID NO:712. In some aspects, the antibody comprises a CDR-H3 sequence comprising, consisting of, or consisting essentially of SEQ ID NO: 713. In some aspects, the antibody comprises a CDR-H3 sequence comprising, consisting of, or consisting essentially of SEQ ID NO:714. In some aspects, the antibody comprises a CDR-H3 sequence comprising, consisting of, or consisting essentially of SEQ ID NO: 715. In some aspects, the antibody comprises a CDR-H3 sequence comprising, consisting of, or consisting essentially of SEQ ID NO:716. In some aspects, the antibody comprises a CDR-H3 sequence comprising, consisting of, or consisting essentially of SEQ ID NO: 717. In some aspects, the antibody comprises a CDR-H3 sequence comprising, consisting of, or consisting essentially of SEQ ID NO:718. In some aspects, the antibody comprises a CDR-H3 sequence comprising, consisting of, or consisting essentially of SEQ ID NO: 719. In some aspects, the antibody comprises a CDR-H3 sequence comprising, consisting of, or consisting essentially of SEQ ID NO:720. In some aspects, the antibody comprises a CDR-H3 sequence comprising, consisting of, or consisting essentially of SEQ ID NO: 721. In some aspects, the antibody comprises a CDR-H3 sequence comprising, consisting of, or consisting essentially of SEQ ID NO:722. In some aspects, the antibody comprises a CDR-H3 sequence comprising, consisting of, or consisting essentially of SEQ ID NO: 723. In some aspects, the antibody comprises a CDR-H3 sequence comprising, consisting of, or consisting essentially of SEQ ID NO:724. In some aspects, the antibody comprises a CDR-H3 sequence comprising, consisting of, or consisting essentially of SEQ ID NO: 725. In some aspects, the antibody comprises a CDR-H3 sequence comprising, consisting of, or consisting essentially of SEQ ID NO:726. In some aspects, the antibody comprises a CDR-H3 sequence comprising, consisting of, or consisting essentially of SEQ ID NO: 727. In some aspects, the antibody comprises a CDR-H3 sequence comprising, consisting of, or consisting essentially of SEQ ID NO:728. In some aspects, the antibody comprises a CDR-H3 sequence comprising, consisting of, or consisting essentially of SEQ ID NO: 729. In some aspects, the antibody comprises a CDR-H3 sequence comprising, consisting of, or consisting essentially of SEQ ID NO:730. In some aspects, the antibody comprises a CDR-H3 sequence comprising, consisting of, or consisting essentially of SEQ ID NO: 731. In some aspects, the antibody comprises a CDR-H3 sequence comprising, consisting of, or consisting essentially of SEQ ID NO:732. In some aspects, the antibody comprises a CDR-H3 sequence comprising, consisting of, or consisting essentially of SEQ ID NO: 733. In some aspects, the antibody comprises a CDR-H3 sequence comprising, consisting of, or consisting essentially of SEQ ID NO:734. In some aspects, the antibody comprises a CDR-H3 sequence comprising, consisting of, or consisting essentially of SEQ ID NO: 735. In some aspects, the antibody comprises a CDR-H3 sequence comprising, consisting of, or consisting essentially of SEQ ID NO:736. In some aspects, the antibody comprises a CDR-H3 sequence comprising, consisting of, or consisting essentially of SEQ ID NO: 737. In some aspects, the antibody comprises a CDR-H3 sequence comprising, consisting of, or consisting essentially of SEQ ID NO:738. In some aspects, the antibody comprises a CDR-H3 sequence comprising, consisting of, or consisting essentially of SEQ ID NO: 739. In some aspects, the antibody comprises a CDR-H3 sequence comprising, consisting of, or consisting essentially of SEQ ID NO:740. In some aspects, the antibody comprises a CDR-H3 sequence comprising, consisting of, or consisting essentially of SEQ ID NO: 741. In some aspects, the antibody comprises a CDR-H3 sequence comprising, consisting of, or consisting essentially of SEQ ID NO:742. In some aspects, the antibody comprises a CDR-H3 sequence comprising, consisting of, or consisting essentially of SEQ ID NO: 743. In some aspects, the antibody comprises a CDR-H3 sequence comprising, consisting of, or consisting essentially of SEQ ID NO:744. In some aspects, the antibody comprises a CDR-H3 sequence comprising, consisting of, or consisting essentially of SEQ ID NO: 745. In some aspects, the antibody comprises a CDR-H3 sequence comprising, consisting of, or consisting essentially of SEQ ID NO:746. In some aspects, the antibody comprises a CDR-H3 sequence comprising, consisting of, or consisting essentially of SEQ ID NO: 747. In some aspects, the antibody comprises a CDR-H3 sequence comprising, consisting of, or consisting essentially of SEQ ID NO:748. In some aspects, the antibody comprises a CDR-H3 sequence comprising, consisting of, or consisting essentially of SEQ ID NO: 749.

In some aspects, the antibody comprises a CDR-H3 sequence comprising, consisting of, or consisting essentially of SEQ ID NO:750. In some aspects, the antibody comprises a CDR-H3 sequence comprising, consisting of, or consisting essentially of SEQ ID NO: 751. In some aspects, the antibody comprises a CDR-H3 sequence comprising, consisting of, or consisting essentially of SEQ ID NO:752. In some aspects, the antibody comprises a CDR-H3 sequence comprising, consisting of, or consisting essentially of SEQ ID NO: 753. In some aspects, the antibody comprises a CDR-H3 sequence comprising, consisting of, or consisting essentially of SEQ ID NO:754. In some aspects, the antibody comprises a CDR-H3 sequence comprising, consisting of, or consisting essentially of SEQ ID NO: 755. In some aspects, the antibody comprises a CDR-H3 sequence comprising, consisting of, or consisting essentially of SEQ ID NO:756. In some aspects, the antibody comprises a CDR-H3 sequence comprising, consisting of, or consisting essentially of SEQ ID NO: 757. In some aspects, the antibody comprises a CDR-H3 sequence comprising, consisting of, or consisting essentially of SEQ ID NO:758. In some aspects, the antibody comprises a CDR-H3 sequence comprising, consisting of, or consisting essentially of SEQ ID NO: 759. In some aspects, the antibody comprises a CDR-H3 sequence comprising, consisting of, or consisting essentially of SEQ ID NO:760. In some aspects, the antibody comprises a CDR-H3 sequence comprising, consisting of, or consisting essentially of SEQ ID NO: 761. In some aspects, the antibody comprises a CDR-H3 sequence comprising, consisting of, or consisting essentially of SEQ ID NO:762. In some aspects, the antibody comprises a CDR-H3 sequence comprising, consisting of, or consisting essentially of SEQ ID NO: 763. In some aspects, the antibody comprises a CDR-H3 sequence comprising, consisting of, or consisting essentially of SEQ ID NO:764. In some aspects, the antibody comprises a CDR-H3 sequence comprising, consisting of, or consisting essentially of SEQ ID NO: 765. In some aspects, the antibody comprises a CDR-H3 sequence comprising, consisting of, or consisting essentially of SEQ ID NO:766. In some aspects, the antibody comprises a CDR-H3 sequence comprising, consisting of, or consisting essentially of SEQ ID NO: 767. In some aspects, the antibody comprises a CDR-H3 sequence comprising, consisting of, or consisting essentially of SEQ ID NO:768. In some aspects, the antibody comprises a CDR-H3 sequence comprising, consisting of, or consisting essentially of SEQ ID NO: 769. In some aspects, the antibody comprises a CDR-H3 sequence comprising, consisting of, or consisting essentially of SEQ ID NO:770. In some aspects, the antibody comprises a CDR-H3 sequence comprising, consisting of, or consisting essentially of SEQ ID NO: 771. In some aspects, the antibody comprises a CDR-H3 sequence comprising, consisting of, or consisting essentially of SEQ ID NO:772. In some aspects, the antibody comprises a CDR-H3 sequence comprising, consisting of, or consisting essentially of SEQ ID NO: 773. In some aspects, the antibody comprises a CDR-H3 sequence comprising, consisting of, or consisting essentially of SEQ ID NO:774. In some aspects, the antibody comprises a CDR-H3 sequence comprising, consisting of, or consisting essentially of SEQ ID NO: 775. In some aspects, the antibody comprises a CDR-H3 sequence comprising, consisting of, or consisting essentially of SEQ ID NO:776. In some aspects, the antibody comprises a CDR-H3 sequence comprising, consisting of, or consisting essentially of SEQ ID NO: 777. In some aspects, the antibody comprises a CDR-H3 sequence comprising, consisting of, or consisting essentially of SEQ ID NO:778. In some aspects, the antibody comprises a CDR-H3 sequence comprising, consisting of, or consisting essentially of SEQ ID NO: 779. In some aspects, the antibody comprises a CDR-H3 sequence comprising, consisting of, or consisting essentially of SEQ ID NO:780. In some aspects, the antibody comprises a CDR-H3 sequence comprising, consisting of, or consisting essentially of SEQ ID NO: 781. In some aspects, the antibody comprises a CDR-H3 sequence comprising, consisting of, or consisting essentially of SEQ ID NO:782. In some aspects, the antibody comprises a CDR-H3 sequence comprising, consisting of, or consisting essentially of SEQ ID NO: 783. In some aspects, the antibody comprises a CDR-H3 sequence comprising, consisting of, or consisting essentially of SEQ ID NO:784. In some aspects, the antibody comprises a CDR-H3 sequence comprising, consisting of, or consisting essentially of SEQ ID NO: 785. In some aspects, the antibody comprises a CDR-H3 sequence comprising, consisting of, or consisting essentially of SEQ ID NO:786. In some aspects, the antibody comprises a CDR-H3 sequence comprising, consisting of, or consisting essentially of SEQ ID NO: 787. In some aspects, the antibody comprises a CDR-H3 sequence comprising, consisting of, or consisting essentially of SEQ ID NO:788. In some aspects, the antibody comprises a CDR-H3 sequence comprising, consisting of, or consisting essentially of SEQ ID NO: 789. In some aspects, the antibody comprises a CDR-H3 sequence comprising, consisting of, or consisting essentially of SEQ ID NO:790. In some aspects, the antibody comprises a CDR-H3 sequence comprising, consisting of, or consisting essentially of SEQ ID NO: 791. In some aspects, the antibody comprises a CDR-H3 sequence comprising, consisting of, or consisting essentially of SEQ ID NO:792. In some aspects, the antibody comprises a CDR-H3 sequence comprising, consisting of, or consisting essentially of SEQ ID NO: 793. In some aspects, the antibody comprises a CDR-H3 sequence comprising, consisting of, or consisting essentially of SEQ ID NO:794. In some aspects, the antibody comprises a CDR-H3 sequence comprising, consisting of, or consisting essentially of SEQ ID NO: 795. In some aspects, the antibody comprises a CDR-H3 sequence comprising, consisting of, or consisting essentially of SEQ ID NO:796. In some aspects, the antibody comprises a CDR-H3 sequence comprising, consisting of, or consisting essentially of SEQ ID NO: 797. In some aspects, the antibody comprises a CDR-H3 sequence comprising, consisting of, or consisting essentially of SEQ ID NO:798. In some aspects, the antibody comprises a CDR-H3 sequence comprising, consisting of, or consisting essentially of SEQ ID NO: 799.

In some aspects, the antibody comprises a CDR-H3 sequence comprising, consisting of, or consisting essentially of SEQ ID NO:800. In some aspects, the antibody comprises a CDR-H3 sequence comprising, consisting of, or consisting essentially of SEQ ID NO: 801. In some aspects, the antibody comprises a CDR-H3 sequence comprising, consisting of, or consisting essentially of SEQ ID NO:802. In some aspects, the antibody comprises a CDR-H3 sequence comprising, consisting of, or consisting essentially of SEQ ID NO: 803. In some aspects, the antibody comprises a CDR-H3 sequence comprising, consisting of, or consisting essentially of SEQ ID NO:804. In some aspects, the antibody comprises a CDR-H3 sequence comprising, consisting of, or consisting essentially of SEQ ID NO: 805. In some aspects, the antibody comprises a CDR-H3 sequence comprising, consisting of, or consisting essentially of SEQ ID NO:806. In some aspects, the antibody comprises a CDR-H3 sequence comprising, consisting of, or consisting essentially of SEQ ID NO: 807. In some aspects, the antibody comprises a CDR-H3 sequence comprising, consisting of, or consisting essentially of SEQ ID NO:808. In some aspects, the antibody comprises a CDR-H3 sequence comprising, consisting of, or consisting essentially of SEQ ID NO: 809. In some aspects, the antibody comprises a CDR-H3 sequence comprising, consisting of, or consisting essentially of SEQ ID NO:810. In some aspects, the antibody comprises a CDR-H3 sequence comprising, consisting of, or consisting essentially of SEQ ID NO: 811. In some aspects, the antibody comprises a CDR-H3 sequence comprising, consisting of, or consisting essentially of SEQ ID NO:812. In some aspects, the antibody comprises a CDR-H3 sequence comprising, consisting of, or consisting essentially of SEQ ID NO: 813. In some aspects, the antibody comprises a CDR-H3 sequence comprising, consisting of, or consisting essentially of SEQ ID NO:814. In some aspects, the antibody comprises a CDR-H3 sequence comprising, consisting of, or consisting essentially of SEQ ID NO: 815. In some aspects, the antibody comprises a CDR-H3 sequence comprising, consisting of, or consisting essentially of SEQ ID NO:816. In some aspects, the antibody comprises a CDR-H3 sequence comprising, consisting of, or consisting essentially of SEQ ID NO: 817. In some aspects, the antibody comprises a CDR-H3 sequence comprising, consisting of, or consisting essentially of SEQ ID NO:818. In some aspects, the antibody comprises a CDR-H3 sequence comprising, consisting of, or consisting essentially of SEQ ID NO: 819. In some aspects, the antibody comprises a CDR-H3 sequence comprising, consisting of, or consisting essentially of SEQ ID NO:820. In some aspects, the antibody comprises a CDR-H3 sequence comprising, consisting of, or consisting essentially of SEQ ID NO: 821. In some aspects, the antibody comprises a CDR-H3 sequence comprising, consisting of, or consisting essentially of SEQ ID NO:822. In some aspects, the antibody comprises a CDR-H3 sequence comprising, consisting of, or consisting essentially of SEQ ID NO: 823. In some aspects, the antibody comprises a CDR-H3 sequence comprising, consisting of, or consisting essentially of SEQ ID NO:824. In some aspects, the antibody comprises a CDR-H3 sequence comprising, consisting of, or consisting essentially of SEQ ID NO: 825. In some aspects, the antibody comprises a CDR-H3 sequence comprising, consisting of, or consisting essentially of SEQ ID NO:826. In some aspects, the antibody comprises a CDR-H3 sequence comprising, consisting of, or consisting essentially of SEQ ID NO: 827. In some aspects, the antibody comprises a CDR-H3 sequence comprising, consisting of, or consisting essentially of SEQ ID NO:828. In some aspects, the antibody comprises a CDR-H3 sequence comprising, consisting of, or consisting essentially of SEQ ID NO: 829. In some aspects, the antibody comprises a CDR-H3 sequence comprising, consisting of, or consisting essentially of SEQ ID NO:830. In some aspects, the antibody comprises a CDR-H3 sequence comprising, consisting of, or consisting essentially of SEQ ID NO: 831. In some aspects, the antibody comprises a CDR-H3 sequence comprising, consisting of, or consisting essentially of SEQ ID NO:832. In some aspects, the antibody comprises a CDR-H3 sequence comprising, consisting of, or consisting essentially of SEQ ID NO: 833. In some aspects, the antibody comprises a CDR-H3 sequence comprising, consisting of, or consisting essentially of SEQ ID NO:834. In some aspects, the antibody comprises a CDR-H3 sequence comprising, consisting of, or consisting essentially of SEQ ID NO: 835. In some aspects, the antibody comprises a CDR-H3 sequence comprising, consisting of, or consisting essentially of SEQ ID NO:836. In some aspects, the antibody comprises a CDR-H3 sequence comprising, consisting of, or consisting essentially of SEQ ID NO: 837. In some aspects, the antibody comprises a CDR-H3 sequence comprising, consisting of, or consisting essentially of SEQ ID NO:838.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

### 5.2 V_{H} Sequences Comprising Illustrative CDRs

In some embodiments, the antibody comprises a V_{H} sequence comprising one or more CDR-H3 sequences comprising, consisting of, or consisting essentially of one or more illustrative CDR-H3 sequences provided in this disclosure, and variants thereof. In some embodiments, the CDR-H3 sequences comprise, consist of, or consist essentially of one or more CDR-H3 sequences provided in a V_{H} sequence selected from SEQ ID NOs: 854-1020.

### 5.2.1. V_{H} Sequences Comprising Illustrative Kabat CDRs

In some embodiments, the antibody comprises a V_{H} sequence comprising one or more Kabat CDR-H3 sequences comprising, consisting of, or consisting essentially of one or more illustrative Kabat CDR-H3 sequences provided in this disclosure, and variants thereof.

### 5.2.1.1. Kabat CDR-H3

In some embodiments, the antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a V_{H} sequence provided in SEQ ID NOs: 854-1020.

In some embodiments, the antibody comprises a V_{H} sequence comprising a Kabat CDR-H3 sequence comprising, consisting of, or consisting essentially of a sequence selected from SEQ ID NOs: 672-838. In some aspects, the antibody comprises a CDR-H3 sequence comprising, consisting of, or consisting essentially of SEQ ID NO:672. In some aspects, the antibody comprises a CDR-H3 sequence comprising, consisting of, or consisting essentially of SEQ ID NO:673. In some aspects, the antibody comprises a CDR-H3 sequence comprising, consisting of, or consisting essentially of SEQ ID NO:674. In some aspects, the antibody comprises a CDR-H3 sequence comprising, consisting of, or consisting essentially of SEQ ID NO:675. In some aspects, the antibody comprises a CDR-H3 sequence comprising, consisting of, or consisting essentially of SEQ ID NO:676. In some aspects, the antibody comprises a CDR-H3 sequence comprising, consisting of, or consisting essentially of SEQ ID NO:677. In some aspects, the antibody comprises a CDR-H3 sequence comprising, consisting of, or consisting essentially of SEQ ID NO:678. In some aspects, the antibody comprises a CDR-H3 sequence comprising, consisting of, or consisting essentially of SEQ ID NO:679. In some aspects, the antibody comprises a CDR-H3 sequence comprising, consisting of, or consisting essentially of SEQ ID NO:680. In some aspects, the antibody comprises a CDR-H3 sequence comprising, consisting of, or consisting essentially of SEQ ID NO:681. In some aspects, the antibody comprises a CDR-H3 sequence comprising, consisting of, or consisting essentially of SEQ ID NO:682. In some aspects, the antibody comprises a CDR-H3 sequence comprising, consisting of, or consisting essentially of SEQ ID NO:683. In some aspects, the antibody comprises a CDR-H3 sequence comprising, consisting of, or consisting essentially of SEQ ID NO:684. In some aspects, the antibody comprises a CDR-H3 sequence comprising, consisting of, or consisting essentially of SEQ ID NO:685. In some aspects, the antibody comprises a CDR-H3 sequence comprising, consisting of, or consisting essentially of SEQ ID NO:686. In some aspects, the antibody comprises a CDR-H3 sequence comprising, consisting of, or consisting essentially of SEQ ID NO:687. In some aspects, the antibody comprises a CDR-H3 sequence comprising, consisting of, or consisting essentially of SEQ ID NO:688. In some aspects, the antibody comprises a CDR-H3 sequence comprising, consisting of, or consisting essentially of SEQ ID NO:689. In some aspects, the antibody comprises a CDR-H3 sequence comprising, consisting of, or consisting essentially of SEQ ID NO:690. In some aspects, the antibody comprises a CDR-H3 sequence comprising, consisting of, or consisting essentially of SEQ ID NO:691. In some aspects, the antibody comprises a CDR-H3 sequence comprising, consisting of, or consisting essentially of SEQ ID NO:692. In some aspects, the antibody comprises a CDR-H3 sequence comprising, consisting of, or consisting essentially of SEQ ID NO:693. In some aspects, the antibody comprises a CDR-H3 sequence comprising, consisting of, or consisting essentially of SEQ ID NO:694. In some aspects, the antibody comprises a CDR-H3 sequence comprising, consisting of, or consisting essentially of SEQ ID NO:695. In some aspects, the antibody comprises a CDR-H3 sequence comprising, consisting of, or consisting essentially of SEQ ID NO:696. In some aspects, the antibody comprises a CDR-H3 sequence comprising, consisting of, or consisting essentially of SEQ ID NO:697. In some aspects, the antibody comprises a CDR-H3 sequence comprising, consisting of, or consisting essentially of SEQ ID NO:698. In some aspects, the antibody comprises a CDR-H3 sequence comprising, consisting of, or consisting essentially of SEQ ID NO:699.

In some aspects, the antibody comprises a CDR-H3 sequence comprising, consisting of, or consisting essentially of SEQ ID NO:700. In some aspects, the antibody comprises a CDR-H3 sequence comprising, consisting of, or consisting essentially of SEQ ID NO: 701. In some aspects, the antibody comprises a CDR-H3 sequence comprising, consisting of, or consisting essentially of SEQ ID NO:702. In some aspects, the antibody comprises a CDR-H3 sequence comprising, consisting of, or consisting essentially of SEQ ID NO: 703. In some aspects, the antibody comprises a CDR-H3 sequence comprising, consisting of, or consisting essentially of SEQ ID NO:704. In some aspects, the antibody comprises a CDR-H3 sequence comprising, consisting of, or consisting essentially of SEQ ID NO: 705. In some aspects, the antibody comprises a CDR-H3 sequence comprising, consisting of, or consisting essentially of SEQ ID NO:706. In some aspects, the antibody comprises a CDR-H3 sequence comprising, consisting of, or consisting essentially of SEQ ID NO: 707. In some aspects, the antibody comprises a CDR-H3 sequence comprising, consisting of, or consisting essentially of SEQ ID NO:708. In some aspects, the antibody comprises a CDR-H3 sequence comprising, consisting of, or consisting essentially of SEQ ID NO: 709. In some aspects, the antibody comprises a CDR-H3 sequence comprising, consisting of, or consisting essentially of SEQ ID NO:710. In some aspects, the antibody comprises a CDR-H3 sequence comprising, consisting of, or consisting essentially of SEQ ID NO: 711. In some aspects, the antibody comprises a CDR-H3 sequence comprising, consisting of, or consisting essentially of SEQ ID NO:712. In some aspects, the antibody comprises a CDR-H3 sequence comprising, consisting of, or consisting essentially of SEQ ID NO: 713. In some aspects, the antibody comprises a CDR-H3 sequence comprising, consisting of, or consisting essentially of SEQ ID NO:714. In some aspects, the antibody comprises a CDR-H3 sequence comprising, consisting of, or consisting essentially of SEQ ID NO: 715. In some aspects, the antibody comprises a CDR-H3 sequence comprising, consisting of, or consisting essentially of SEQ ID NO:716. In some aspects, the antibody comprises a CDR-H3 sequence comprising, consisting of, or consisting essentially of SEQ ID NO: 717. In some aspects, the antibody comprises a CDR-H3 sequence comprising, consisting of, or consisting essentially of SEQ ID NO:718. In some aspects, the antibody comprises a CDR-H3 sequence comprising, consisting of, or consisting essentially of SEQ ID NO: 719. In some aspects, the antibody comprises a CDR-H3 sequence comprising, consisting of, or consisting essentially of SEQ ID NO:720. In some aspects, the antibody comprises a CDR-H3 sequence comprising, consisting of, or consisting essentially of SEQ ID NO: 721. In some aspects, the antibody comprises a CDR-H3 sequence comprising, consisting of, or consisting essentially of SEQ ID NO:722. In some aspects, the antibody comprises a CDR-H3 sequence comprising, consisting of, or consisting essentially of SEQ ID NO: 723. In some aspects, the antibody comprises a CDR-H3 sequence comprising, consisting of, or consisting essentially of SEQ ID NO:724. In some aspects, the antibody comprises a CDR-H3 sequence comprising, consisting of, or consisting essentially of SEQ ID NO: 725. In some aspects, the antibody comprises a CDR-H3 sequence comprising, consisting of, or consisting essentially of SEQ ID NO:726. In some aspects, the antibody comprises a CDR-H3 sequence comprising, consisting of, or consisting essentially of SEQ ID NO: 727. In some aspects, the antibody comprises a CDR-H3 sequence comprising, consisting of, or consisting essentially of SEQ ID NO:728. In some aspects, the antibody comprises a CDR-H3 sequence comprising, consisting of, or consisting essentially of SEQ ID NO: 729. In some aspects, the antibody comprises a CDR-H3 sequence comprising, consisting of, or consisting essentially of SEQ ID NO:730. In some aspects, the antibody comprises a CDR-H3 sequence comprising, consisting of, or consisting essentially of SEQ ID NO: 731. In some aspects, the antibody comprises a CDR-H3 sequence comprising, consisting of, or consisting essentially of SEQ ID NO:732. In some aspects, the antibody comprises a CDR-H3 sequence comprising, consisting of, or consisting essentially of SEQ ID NO: 733. In some aspects, the antibody comprises a CDR-H3 sequence comprising, consisting of, or consisting essentially of SEQ ID NO:734. In some aspects, the antibody comprises a CDR-H3 sequence comprising, consisting of, or consisting essentially of SEQ ID NO: 735. In some aspects, the antibody comprises a CDR-H3 sequence comprising, consisting of, or consisting essentially of SEQ ID NO:736. In some aspects, the antibody comprises a CDR-H3 sequence comprising, consisting of, or consisting essentially of SEQ ID NO: 737. In some aspects, the antibody comprises a CDR-H3 sequence comprising, consisting of, or consisting essentially of SEQ ID NO:738. In some aspects, the antibody comprises a CDR-H3 sequence comprising, consisting of, or consisting essentially of SEQ ID NO: 739. In some aspects, the antibody comprises a CDR-H3 sequence comprising, consisting of, or consisting essentially of SEQ ID NO:740. In some aspects, the antibody comprises a CDR-H3 sequence comprising, consisting of, or consisting essentially of SEQ ID NO: 741. In some aspects, the antibody comprises a CDR-H3 sequence comprising, consisting of, or consisting essentially of SEQ ID NO:742. In some aspects, the antibody comprises a CDR-H3 sequence comprising, consisting of, or consisting essentially of SEQ ID NO: 743. In some aspects, the antibody comprises a CDR-H3 sequence comprising, consisting of, or consisting essentially of SEQ ID NO:744. In some aspects, the antibody comprises a CDR-H3 sequence comprising, consisting of, or consisting essentially of SEQ ID NO: 745. In some aspects, the antibody comprises a CDR-H3 sequence comprising, consisting of, or consisting essentially of SEQ ID NO:746. In some aspects, the antibody comprises a CDR-H3 sequence comprising, consisting of, or consisting essentially of SEQ ID NO: 747. In some aspects, the antibody comprises a CDR-H3 sequence comprising, consisting of, or consisting essentially of SEQ ID NO:748. In some aspects, the antibody comprises a CDR-H3 sequence comprising, consisting of, or consisting essentially of SEQ ID NO: 749.

In some aspects, the antibody comprises a CDR-H3 sequence comprising, consisting of, or consisting essentially of SEQ ID NO:750. In some aspects, the antibody comprises a CDR-H3 sequence comprising, consisting of, or consisting essentially of SEQ ID NO: 751. In some aspects, the antibody comprises a CDR-H3 sequence comprising, consisting of, or consisting essentially of SEQ ID NO:752. In some aspects, the antibody comprises a CDR-H3 sequence comprising, consisting of, or consisting essentially of SEQ ID NO: 753. In some aspects, the antibody comprises a CDR-H3 sequence comprising, consisting of, or consisting essentially of SEQ ID NO:754. In some aspects, the antibody comprises a CDR-H3 sequence comprising, consisting of, or consisting essentially of SEQ ID NO: 755. In some aspects, the antibody comprises a CDR-H3 sequence comprising, consisting of, or consisting essentially of SEQ ID NO:756. In some aspects, the antibody comprises a CDR-H3 sequence comprising, consisting of, or consisting essentially of SEQ ID NO: 757. In some aspects, the antibody comprises a CDR-H3 sequence comprising, consisting of, or consisting essentially of SEQ ID NO:758. In some aspects, the antibody comprises a CDR-H3 sequence comprising, consisting of, or consisting essentially of SEQ ID NO: 759. In some aspects, the antibody comprises a CDR-H3 sequence comprising, consisting of, or consisting essentially of SEQ ID NO:760. In some aspects, the antibody comprises a CDR-H3 sequence comprising, consisting of, or consisting essentially of SEQ ID NO: 761. In some aspects, the antibody comprises a CDR-H3 sequence comprising, consisting of, or consisting essentially of SEQ ID NO:762. In some aspects, the antibody comprises a CDR-H3 sequence comprising, consisting of, or consisting essentially of SEQ ID NO: 763. In some aspects, the antibody comprises a CDR-H3 sequence comprising, consisting of, or consisting essentially of SEQ ID NO:764. In some aspects, the antibody comprises a CDR-H3 sequence comprising, consisting of, or consisting essentially of SEQ ID NO: 765. In some aspects, the antibody comprises a CDR-H3 sequence comprising, consisting of, or consisting essentially of SEQ ID NO:766. In some aspects, the antibody comprises a CDR-H3 sequence comprising, consisting of, or consisting essentially of SEQ ID NO: 767. In some aspects, the antibody comprises a CDR-H3 sequence comprising, consisting of, or consisting essentially of SEQ ID NO:768. In some aspects, the antibody comprises a CDR-H3 sequence comprising, consisting of, or consisting essentially of SEQ ID NO: 769. In some aspects, the antibody comprises a CDR-H3 sequence comprising, consisting of, or consisting essentially of SEQ ID NO:770. In some aspects, the antibody comprises a CDR-H3 sequence comprising, consisting of, or consisting essentially of SEQ ID NO: 771. In some aspects, the antibody comprises a CDR-H3 sequence comprising, consisting of, or consisting essentially of SEQ ID NO:772. In some aspects, the antibody comprises a CDR-H3 sequence comprising, consisting of, or consisting essentially of SEQ ID NO: 773. In some aspects, the antibody comprises a CDR-H3 sequence comprising, consisting of, or consisting essentially of SEQ ID NO:774. In some aspects, the antibody comprises a CDR-H3 sequence comprising, consisting of, or consisting essentially of SEQ ID NO: 775. In some aspects, the antibody comprises a CDR-H3 sequence comprising, consisting of, or consisting essentially of SEQ ID NO:776. In some aspects, the antibody comprises a CDR-H3 sequence comprising, consisting of, or consisting essentially of SEQ ID NO: 777. In some aspects, the antibody comprises a CDR-H3 sequence comprising, consisting of, or consisting essentially of SEQ ID NO:778. In some aspects, the antibody comprises a CDR-H3 sequence comprising, consisting of, or consisting essentially of SEQ ID NO: 779. In some aspects, the antibody comprises a CDR-H3 sequence comprising, consisting of, or consisting essentially of SEQ ID NO:780. In some aspects, the antibody comprises a CDR-H3 sequence comprising, consisting of, or consisting essentially of SEQ ID NO: 781. In some aspects, the antibody comprises a CDR-H3 sequence comprising, consisting of, or consisting essentially of SEQ ID NO:782. In some aspects, the antibody comprises a CDR-H3 sequence comprising, consisting of, or consisting essentially of SEQ ID NO: 783. In some aspects, the antibody comprises a CDR-H3 sequence comprising, consisting of, or consisting essentially of SEQ ID NO:784. In some aspects, the antibody comprises a CDR-H3 sequence comprising, consisting of, or consisting essentially of SEQ ID NO: 785. In some aspects, the antibody comprises a CDR-H3 sequence comprising, consisting of, or consisting essentially of SEQ ID NO:786. In some aspects, the antibody comprises a CDR-H3 sequence comprising, consisting of, or consisting essentially of SEQ ID NO: 787. In some aspects, the antibody comprises a CDR-H3 sequence comprising, consisting of, or consisting essentially of SEQ ID NO:788. In some aspects, the antibody comprises a CDR-H3 sequence comprising, consisting of, or consisting essentially of SEQ ID NO: 789. In some aspects, the antibody comprises a CDR-H3 sequence comprising, consisting of, or consisting essentially of SEQ ID NO:790. In some aspects, the antibody comprises a CDR-H3 sequence comprising, consisting of, or consisting essentially of SEQ ID NO: 791. In some aspects, the antibody comprises a CDR-H3 sequence comprising, consisting of, or consisting essentially of SEQ ID NO:792. In some aspects, the antibody comprises a CDR-H3 sequence comprising, consisting of, or consisting essentially of SEQ ID NO: 793. In some aspects, the antibody comprises a CDR-H3 sequence comprising, consisting of, or consisting essentially of SEQ ID NO:794. In some aspects, the antibody comprises a CDR-H3 sequence comprising, consisting of, or consisting essentially of SEQ ID NO: 795. In some aspects, the antibody comprises a CDR-H3 sequence comprising, consisting of, or consisting essentially of SEQ ID NO:796. In some aspects, the antibody comprises a CDR-H3 sequence comprising, consisting of, or consisting essentially of SEQ ID NO: 797. In some aspects, the antibody comprises a CDR-H3 sequence comprising, consisting of, or consisting essentially of SEQ ID NO:798. In some aspects, the antibody comprises a CDR-H3 sequence comprising, consisting of, or consisting essentially of SEQ ID NO: 799.

In some aspects, the antibody comprises a CDR-H3 sequence comprising, consisting of, or consisting essentially of SEQ ID NO:800. In some aspects, the antibody comprises a CDR-H3 sequence comprising, consisting of, or consisting essentially of SEQ ID NO: 801. In some aspects, the antibody comprises a CDR-H3 sequence comprising, consisting of, or consisting essentially of SEQ ID NO:802. In some aspects, the antibody comprises a CDR-H3 sequence comprising, consisting of, or consisting essentially of SEQ ID NO: 803. In some aspects, the antibody comprises a CDR-H3 sequence comprising, consisting of, or consisting essentially of SEQ ID NO:804. In some aspects, the antibody comprises a CDR-H3 sequence comprising, consisting of, or consisting essentially of SEQ ID NO: 805. In some aspects, the antibody comprises a CDR-H3 sequence comprising, consisting of, or consisting essentially of SEQ ID NO:806. In some aspects, the antibody comprises a CDR-H3 sequence comprising, consisting of, or consisting essentially of SEQ ID NO: 807. In some aspects, the antibody comprises a CDR-H3 sequence comprising, consisting of, or consisting essentially of SEQ ID NO:808. In some aspects, the antibody comprises a CDR-H3 sequence comprising, consisting of, or consisting essentially of SEQ ID NO: 809. In some aspects, the antibody comprises a CDR-H3 sequence comprising, consisting of, or consisting essentially of SEQ ID NO:810. In some aspects, the antibody comprises a CDR-H3 sequence comprising, consisting of, or consisting essentially of SEQ ID NO: 811. In some aspects, the antibody comprises a CDR-H3 sequence comprising, consisting of, or consisting essentially of SEQ ID NO:812. In some aspects, the antibody comprises a CDR-H3 sequence comprising, consisting of, or consisting essentially of SEQ ID NO: 813. In some aspects, the antibody comprises a CDR-H3 sequence comprising, consisting of, or consisting essentially of SEQ ID NO:814. In some aspects, the antibody comprises a CDR-H3 sequence comprising, consisting of, or consisting essentially of SEQ ID NO: 815. In some aspects, the antibody comprises a CDR-H3 sequence comprising, consisting of, or consisting essentially of SEQ ID NO:816. In some aspects, the antibody comprises a CDR-H3 sequence comprising, consisting of, or consisting essentially of SEQ ID NO: 817. In some aspects, the antibody comprises a CDR-H3 sequence comprising, consisting of, or consisting essentially of SEQ ID NO:818. In some aspects, the antibody comprises a CDR-H3 sequence comprising, consisting of, or consisting essentially of SEQ ID NO: 819. In some aspects, the antibody comprises a CDR-H3 sequence comprising, consisting of, or consisting essentially of SEQ ID NO:820. In some aspects, the antibody comprises a CDR-H3 sequence comprising, consisting of, or consisting essentially of SEQ ID NO: 821. In some aspects, the antibody comprises a CDR-H3 sequence comprising, consisting of, or consisting essentially of SEQ ID NO:822. In some aspects, the antibody comprises a CDR-H3 sequence comprising, consisting of, or consisting essentially of SEQ ID NO: 823. In some aspects, the antibody comprises a CDR-H3 sequence comprising, consisting of, or consisting essentially of SEQ ID NO:824. In some aspects, the antibody comprises a CDR-H3 sequence comprising, consisting of, or consisting essentially of SEQ ID NO: 825. In some aspects, the antibody comprises a CDR-H3 sequence comprising, consisting of, or consisting essentially of SEQ ID NO:826. In some aspects, the antibody comprises a CDR-H3 sequence comprising, consisting of, or consisting essentially of SEQ ID NO: 827. In some aspects, the antibody comprises a CDR-H3 sequence comprising, consisting of, or consisting essentially of SEQ ID NO:828. In some aspects, the antibody comprises a CDR-H3 sequence comprising, consisting of, or consisting essentially of SEQ ID NO: 829. In some aspects, the antibody comprises a CDR-H3 sequence comprising, consisting of, or consisting essentially of SEQ ID NO:830. In some aspects, the antibody comprises a CDR-H3 sequence comprising, consisting of, or consisting essentially of SEQ ID NO: 831. In some aspects, the antibody comprises a CDR-H3 sequence comprising, consisting of, or consisting essentially of SEQ ID NO:832. In some aspects, the antibody comprises a CDR-H3 sequence comprising, consisting of, or consisting essentially of SEQ ID NO: 833. In some aspects, the antibody comprises a CDR-H3 sequence comprising, consisting of, or consisting essentially of SEQ ID NO:834. In some aspects, the antibody comprises a CDR-H3 sequence comprising, consisting of, or consisting essentially of SEQ ID NO: 835. In some aspects, the antibody comprises a CDR-H3 sequence comprising, consisting of, or consisting essentially of SEQ ID NO:836. In some aspects, the antibody comprises a CDR-H3 sequence comprising, consisting of, or consisting essentially of SEQ ID NO: 837. In some aspects, the antibody comprises a CDR-H3 sequence comprising, consisting of, or consisting essentially of SEQ ID NO:838.

### 5.2.1.2. Kabat CDR-H2

In some embodiments, the antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of a V_{H} sequence provided in SEQ ID NOs: 854-1020.

In some embodiments, the antibody comprises a V_{H} sequence comprising a Kabat CDR-H2 sequence comprising, consisting of, or consisting essentially of a sequence selected from SEQ ID NOs: 505-671. In some aspects, the antibody comprises a Kabat CDR-H2 sequence comprising, consisting of, or consisting essentially of SEQ ID NO:505. In some aspects, the antibody comprises a Kabat CDR-H2 sequence comprising, consisting of, or consisting essentially of SEQ ID NO:506. In some aspects, the antibody comprises a Kabat CDR-H2 sequence comprising, consisting of, or consisting essentially of SEQ ID NO: 507. In some aspects, the antibody comprises a Kabat CDR-H2 sequence comprising, consisting of, or consisting essentially of SEQ ID NO:508. In some aspects, the antibody comprises a Kabat CDR-H2 sequence comprising, consisting of, or consisting essentially of SEQ ID NO:509. In some aspects, the antibody comprises a Kabat CDR-H2 sequence comprising, consisting of, or consisting essentially of SEQ ID NO:510. In some aspects, the antibody comprises a Kabat CDR-H2 sequence comprising, consisting of, or consisting essentially of SEQ ID NO:511. In some aspects, the antibody comprises a Kabat CDR-H2 sequence comprising, consisting of, or consisting essentially of SEQ ID NO:512. In some aspects, the antibody comprises a Kabat CDR-H2 sequence comprising, consisting of, or consisting essentially of SEQ ID NO:513. In some aspects, the antibody comprises a Kabat CDR-H2 sequence comprising, consisting of, or consisting essentially of SEQ ID NO:514. In some aspects, the antibody comprises a Kabat CDR-H2 sequence comprising, consisting of, or consisting essentially of SEQ ID NO:515. In some aspects, the antibody comprises a Kabat CDR-H2 sequence comprising, consisting of, or consisting essentially of SEQ ID NO: 516. In some aspects, the antibody comprises a Kabat CDR-H2 sequence comprising, consisting of, or consisting essentially of SEQ ID NO:517. In some aspects, the antibody comprises a Kabat CDR-H2 sequence comprising, consisting of, or consisting essentially of SEQ ID NO:518. In some aspects, the antibody comprises a Kabat CDR-H2 sequence comprising, consisting of, or consisting essentially of SEQ ID NO:519. In some aspects, the antibody comprises a Kabat CDR-H2 sequence comprising, consisting of, or consisting essentially of SEQ ID NO:520. In some aspects, the antibody comprises a Kabat CDR-H2 sequence comprising, consisting of, or consisting essentially of SEQ ID NO:521. In some aspects, the antibody comprises a Kabat CDR-H2 sequence comprising, consisting of, or consisting essentially of SEQ ID NO:522. In some aspects, the antibody comprises a Kabat CDR-H2 sequence comprising, consisting of, or consisting essentially of SEQ ID NO:523. In some aspects, the antibody comprises a Kabat CDR-H2 sequence comprising, consisting of, or consisting essentially of SEQ ID NO:524. In some aspects, the antibody comprises a Kabat CDR-H2 sequence comprising, consisting of, or consisting essentially of SEQ ID NO: 525. In some aspects, the antibody comprises a Kabat CDR-H2 sequence comprising, consisting of, or consisting essentially of SEQ ID NO:526. In some aspects, the antibody comprises a Kabat CDR-H2 sequence comprising, consisting of, or consisting essentially of SEQ ID NO:527. In some aspects, the antibody comprises a Kabat CDR-H2 sequence comprising, consisting of, or consisting essentially of SEQ ID NO:528. In some aspects, the antibody comprises a Kabat CDR-H2 sequence comprising, consisting of, or consisting essentially of SEQ ID NO:529. In some aspects, the antibody comprises a Kabat CDR-H2 sequence comprising, consisting of, or consisting essentially of SEQ ID NO:530. In some aspects, the antibody comprises a Kabat CDR-H2 sequence comprising, consisting of, or consisting essentially of SEQ ID NO:531. In some aspects, the antibody comprises a Kabat CDR-H2 sequence comprising, consisting of, or consisting essentially of SEQ ID NO:532. In some aspects, the antibody comprises a Kabat CDR-H2 sequence comprising, consisting of, or consisting essentially of SEQ ID NO:533. In some aspects, the antibody comprises a Kabat CDR-H2 sequence comprising, consisting of, or consisting essentially of SEQ ID NO: 534. In some aspects, the antibody comprises a Kabat CDR-H2 sequence comprising, consisting of, or consisting essentially of SEQ ID NO:535. In some aspects, the antibody comprises a Kabat CDR-H2 sequence comprising, consisting of, or consisting essentially of SEQ ID NO:536. In some aspects, the antibody comprises a Kabat CDR-H2 sequence comprising, consisting of, or consisting essentially of SEQ ID NO:537. In some aspects, the antibody comprises a Kabat CDR-H2 sequence comprising, consisting of, or consisting essentially of SEQ ID NO:538. In some aspects, the antibody comprises a Kabat CDR-H2 sequence comprising, consisting of, or consisting essentially of SEQ ID NO:539. In some aspects, the antibody comprises a Kabat CDR-H2 sequence comprising, consisting of, or consisting essentially of SEQ ID NO:540. In some aspects, the antibody comprises a Kabat CDR-H2 sequence comprising, consisting of, or consisting essentially of SEQ ID NO:541. In some aspects, the antibody comprises a Kabat CDR-H2 sequence comprising, consisting of, or consisting essentially of SEQ ID NO:542. In some aspects, the antibody comprises a Kabat CDR-H2 sequence comprising, consisting of, or consisting essentially of SEQ ID NO: 543. In some aspects, the antibody comprises a Kabat CDR-H2 sequence comprising, consisting of, or consisting essentially of SEQ ID NO:544. In some aspects, the antibody comprises a Kabat CDR-H2 sequence comprising, consisting of, or consisting essentially of SEQ ID NO:545. In some aspects, the antibody comprises a Kabat CDR-H2 sequence comprising, consisting of, or consisting essentially of SEQ ID NO:546. In some aspects, the antibody comprises a Kabat CDR-H2 sequence comprising, consisting of, or consisting essentially of SEQ ID NO:547. In some aspects, the antibody comprises a Kabat CDR-H2 sequence comprising, consisting of, or consisting essentially of SEQ ID NO:548. In some aspects, the antibody comprises a Kabat CDR-H2 sequence comprising, consisting of, or consisting essentially of SEQ ID NO:549.

In some aspects, the antibody comprises a Kabat CDR-H2 sequence comprising, consisting of, or consisting essentially of SEQ ID NO:550. In some aspects, the antibody comprises a Kabat CDR-H2 sequence comprising, consisting of, or consisting essentially of SEQ ID NO:551. In some aspects, the antibody comprises a Kabat CDR-H2 sequence comprising, consisting of, or consisting essentially of SEQ ID NO:552. In some aspects, the antibody comprises a Kabat CDR-H2 sequence comprising, consisting of, or consisting essentially of SEQ ID NO:553. In some aspects, the antibody comprises a Kabat CDR-H2 sequence comprising, consisting of, or consisting essentially of SEQ ID NO:554. In some aspects, the antibody comprises a Kabat CDR-H2 sequence comprising, consisting of, or consisting essentially of SEQ ID NO:555. In some aspects, the antibody comprises a Kabat CDR-H2 sequence comprising, consisting of, or consisting essentially of SEQ ID NO:556. In some aspects, the antibody comprises a Kabat CDR-H2 sequence comprising, consisting of, or consisting essentially of SEQ ID NO:557. In some aspects, the antibody comprises a Kabat CDR-H2 sequence comprising, consisting of, or consisting essentially of SEQ ID NO: 558. In some aspects, the antibody comprises a Kabat CDR-H2 sequence comprising, consisting of, or consisting essentially of SEQ ID NO:559. In some aspects, the antibody comprises a Kabat CDR-H2 sequence comprising, consisting of, or consisting essentially of SEQ ID NO:560. In some aspects, the antibody comprises a Kabat CDR-H2 sequence comprising, consisting of, or consisting essentially of SEQ ID NO:561. In some aspects, the antibody comprises a Kabat CDR-H2 sequence comprising, consisting of, or consisting essentially of SEQ ID NO:562. In some aspects, the antibody comprises a Kabat CDR-H2 sequence comprising, consisting of, or consisting essentially of SEQ ID NO:563. In some aspects, the antibody comprises a Kabat CDR-H2 sequence comprising, consisting of, or consisting essentially of SEQ ID NO:564. In some aspects, the antibody comprises a Kabat CDR-H2 sequence comprising, consisting of, or consisting essentially of SEQ ID NO:565. In some aspects, the antibody comprises a Kabat CDR-H2 sequence comprising, consisting of, or consisting essentially of SEQ ID NO:566. In some aspects, the antibody comprises a Kabat CDR-H2 sequence comprising, consisting of, or consisting essentially of SEQ ID NO: 567. In some aspects, the antibody comprises a Kabat CDR-H2 sequence comprising, consisting of, or consisting essentially of SEQ ID NO:568. In some aspects, the antibody comprises a Kabat CDR-H2 sequence comprising, consisting of, or consisting essentially of SEQ ID NO:569. In some aspects, the antibody comprises a Kabat CDR-H2 sequence comprising, consisting of, or consisting essentially of SEQ ID NO:570. In some aspects, the antibody comprises a Kabat CDR-H2 sequence comprising, consisting of, or consisting essentially of SEQ ID NO:571. In some aspects, the antibody comprises a Kabat CDR-H2 sequence comprising, consisting of, or consisting essentially of SEQ ID NO:572. In some aspects, the antibody comprises a Kabat CDR-H2 sequence comprising, consisting of, or consisting essentially of SEQ ID NO:573. In some aspects, the antibody comprises a Kabat CDR-H2 sequence comprising, consisting of, or consisting essentially of SEQ ID NO:574. In some aspects, the antibody comprises a Kabat CDR-H2 sequence comprising, consisting of, or consisting essentially of SEQ ID NO:575. In some aspects, the antibody comprises a Kabat CDR-H2 sequence comprising, consisting of, or consisting essentially of SEQ ID NO: 576. In some aspects, the antibody comprises a Kabat CDR-H2 sequence comprising, consisting of, or consisting essentially of SEQ ID NO:577. In some aspects, the antibody comprises a Kabat CDR-H2 sequence comprising, consisting of, or consisting essentially of SEQ ID NO:578. In some aspects, the antibody comprises a Kabat CDR-H2 sequence comprising, consisting of, or consisting essentially of SEQ ID NO:579. In some aspects, the antibody comprises a Kabat CDR-H2 sequence comprising, consisting of, or consisting essentially of SEQ ID NO:580. In some aspects, the antibody comprises a Kabat CDR-H2 sequence comprising, consisting of, or consisting essentially of SEQ ID NO:581. In some aspects, the antibody comprises a Kabat CDR-H2 sequence comprising, consisting of, or consisting essentially of SEQ ID NO:582. In some aspects, the antibody comprises a Kabat CDR-H2 sequence comprising, consisting of, or consisting essentially of SEQ ID NO:583. In some aspects, the antibody comprises a Kabat CDR-H2 sequence comprising, consisting of, or consisting essentially of SEQ ID NO:584. In some aspects, the antibody comprises a Kabat CDR-H2 sequence comprising, consisting of, or consisting essentially of SEQ ID NO: 585. In some aspects, the antibody comprises a Kabat CDR-H2 sequence comprising, consisting of, or consisting essentially of SEQ ID NO:586. In some aspects, the antibody comprises a Kabat CDR-H2 sequence comprising, consisting of, or consisting essentially of SEQ ID NO:587. In some aspects, the antibody comprises a Kabat CDR-H2 sequence comprising, consisting of, or consisting essentially of SEQ ID NO:588. In some aspects, the antibody comprises a Kabat CDR-H2 sequence comprising, consisting of, or consisting essentially of SEQ ID NO:589. In some aspects, the antibody comprises a Kabat CDR-H2 sequence comprising, consisting of, or consisting essentially of SEQ ID NO:590. In some aspects, the antibody comprises a Kabat CDR-H2 sequence comprising, consisting of, or consisting essentially of SEQ ID NO:591. In some aspects, the antibody comprises a Kabat CDR-H2 sequence comprising, consisting of, or consisting essentially of SEQ ID NO:592. In some aspects, the antibody comprises a Kabat CDR-H2 sequence comprising, consisting of, or consisting essentially of SEQ ID NO:593. In some aspects, the antibody comprises a Kabat CDR-H2 sequence comprising, consisting of, or consisting essentially of SEQ ID NO: 594. In some aspects, the antibody comprises a Kabat CDR-H2 sequence comprising, consisting of, or consisting essentially of SEQ ID NO:595. In some aspects, the antibody comprises a Kabat CDR-H2 sequence comprising, consisting of, or consisting essentially of SEQ ID NO:596. In some aspects, the antibody comprises a Kabat CDR-H2 sequence comprising, consisting of, or consisting essentially of SEQ ID NO:597. In some aspects, the antibody comprises a Kabat CDR-H2 sequence comprising, consisting of, or consisting essentially of SEQ ID NO:598. In some aspects, the antibody comprises a Kabat CDR-H2 sequence comprising, consisting of, or consisting essentially of SEQ ID NO:599.

In some aspects, the antibody comprises a Kabat CDR-H2 sequence comprising, consisting of, or consisting essentially of SEQ ID NO:600. In some aspects, the antibody comprises a Kabat CDR-H2 sequence comprising, consisting of, or consisting essentially of SEQ ID NO:601. In some aspects, the antibody comprises a Kabat CDR-H2 sequence comprising, consisting of, or consisting essentially of SEQ ID NO:602. In some aspects, the antibody comprises a Kabat CDR-H2 sequence comprising, consisting of, or consisting essentially of SEQ ID NO:603. In some aspects, the antibody comprises a Kabat CDR-H2 sequence comprising, consisting of, or consisting essentially of SEQ ID NO:604. In some aspects, the antibody comprises a Kabat CDR-H2 sequence comprising, consisting of, or consisting essentially of SEQ ID NO:605. In some aspects, the antibody comprises a Kabat CDR-H2 sequence comprising, consisting of, or consisting essentially of SEQ ID NO:606. In some aspects, the antibody comprises a Kabat CDR-H2 sequence comprising, consisting of, or consisting essentially of SEQ ID NO:607. In some aspects, the antibody comprises a Kabat CDR-H2 sequence comprising, consisting of, or consisting essentially of SEQ ID NO: 608. In some aspects, the antibody comprises a Kabat CDR-H2 sequence comprising, consisting of, or consisting essentially of SEQ ID NO:609. In some aspects, the antibody comprises a Kabat CDR-H2 sequence comprising, consisting of, or consisting essentially of SEQ ID NO:610. In some aspects, the antibody comprises a Kabat CDR-H2 sequence comprising, consisting of, or consisting essentially of SEQ ID NO:611. In some aspects, the antibody comprises a Kabat CDR-H2 sequence comprising, consisting of, or consisting essentially of SEQ ID NO:612. In some aspects, the antibody comprises a Kabat CDR-H2 sequence comprising, consisting of, or consisting essentially of SEQ ID NO:613. In some aspects, the antibody comprises a Kabat CDR-H2 sequence comprising, consisting of, or consisting essentially of SEQ ID NO:614. In some aspects, the antibody comprises a Kabat CDR-H2 sequence comprising, consisting of, or consisting essentially of SEQ ID NO:615. In some aspects, the antibody comprises a Kabat CDR-H2 sequence comprising, consisting of, or consisting essentially of SEQ ID NO:616. In some aspects, the antibody comprises a Kabat CDR-H2 sequence comprising, consisting of, or consisting essentially of SEQ ID NO: 617. In some aspects, the antibody comprises a Kabat CDR-H2 sequence comprising, consisting of, or consisting essentially of SEQ ID NO:618. In some aspects, the antibody comprises a Kabat CDR-H2 sequence comprising, consisting of, or consisting essentially of SEQ ID NO:619. In some aspects, the antibody comprises a Kabat CDR-H2 sequence comprising, consisting of, or consisting essentially of SEQ ID NO:620. In some aspects, the antibody comprises a Kabat CDR-H2 sequence comprising, consisting of, or consisting essentially of SEQ ID NO:621. In some aspects, the antibody comprises a Kabat CDR-H2 sequence comprising, consisting of, or consisting essentially of SEQ ID NO:622. In some aspects, the antibody comprises a Kabat CDR-H2 sequence comprising, consisting of, or consisting essentially of SEQ ID NO:623. In some aspects, the antibody comprises a Kabat CDR-H2 sequence comprising, consisting of, or consisting essentially of SEQ ID NO:624. In some aspects, the antibody comprises a Kabat CDR-H2 sequence comprising, consisting of, or consisting essentially of SEQ ID NO:625. In some aspects, the antibody comprises a Kabat CDR-H2 sequence comprising, consisting of, or consisting essentially of SEQ ID NO: 626. In some aspects, the antibody comprises a Kabat CDR-H2 sequence comprising, consisting of, or consisting essentially of SEQ ID NO:627. In some aspects, the antibody comprises a Kabat CDR-H2 sequence comprising, consisting of, or consisting essentially of SEQ ID NO:628. In some aspects, the antibody comprises a Kabat CDR-H2 sequence comprising, consisting of, or consisting essentially of SEQ ID NO:629. In some aspects, the antibody comprises a Kabat CDR-H2 sequence comprising, consisting of, or consisting essentially of SEQ ID NO:630. In some aspects, the antibody comprises a Kabat CDR-H2 sequence comprising, consisting of, or consisting essentially of SEQ ID NO:631. In some aspects, the antibody comprises a Kabat CDR-H2 sequence comprising, consisting of, or consisting essentially of SEQ ID NO:632. In some aspects, the antibody comprises a Kabat CDR-H2 sequence comprising, consisting of, or consisting essentially of SEQ ID NO:633. In some aspects, the antibody comprises a Kabat CDR-H2 sequence comprising, consisting of, or consisting essentially of SEQ ID NO:634. In some aspects, the antibody comprises a Kabat CDR-H2 sequence comprising, consisting of, or consisting essentially of SEQ ID NO: 635. In some aspects, the antibody comprises a Kabat CDR-H2 sequence comprising, consisting of, or consisting essentially of SEQ ID NO:636. In some aspects, the antibody comprises a Kabat CDR-H2 sequence comprising, consisting of, or consisting essentially of SEQ ID NO:637. In some aspects, the antibody comprises a Kabat CDR-H2 sequence comprising, consisting of, or consisting essentially of SEQ ID NO:638. In some aspects, the antibody comprises a Kabat CDR-H2 sequence comprising, consisting of, or consisting essentially of SEQ ID NO:639. In some aspects, the antibody comprises a Kabat CDR-H2 sequence comprising, consisting of, or consisting essentially of SEQ ID NO:640. In some aspects, the antibody comprises a Kabat CDR-H2 sequence comprising, consisting of, or consisting essentially of SEQ ID NO:641. In some aspects, the antibody comprises a Kabat CDR-H2 sequence comprising, consisting of, or consisting essentially of SEQ ID NO:642. In some aspects, the antibody comprises a Kabat CDR-H2 sequence comprising, consisting of, or consisting essentially of SEQ ID NO:643. In some aspects, the antibody comprises a Kabat CDR-H2 sequence comprising, consisting of, or consisting essentially of SEQ ID NO: 644. In some aspects, the antibody comprises a Kabat CDR-H2 sequence comprising, consisting of, or consisting essentially of SEQ ID NO:645. In some aspects, the antibody comprises a Kabat CDR-H2 sequence comprising, consisting of, or consisting essentially of SEQ ID NO:646. In some aspects, the antibody comprises a Kabat CDR-H2 sequence comprising, consisting of, or consisting essentially of SEQ ID NO:647. In some aspects, the antibody comprises a Kabat CDR-H2 sequence comprising, consisting of, or consisting essentially of SEQ ID NO:648. In some aspects, the antibody comprises a Kabat CDR-H2 sequence comprising, consisting of, or consisting essentially of SEQ ID NO:649.

In some aspects, the antibody comprises a Kabat CDR-H2 sequence comprising, consisting of, or consisting essentially of SEQ ID NO:650. In some aspects, the antibody comprises a Kabat CDR-H2 sequence comprising, consisting of, or consisting essentially of SEQ ID NO:651. In some aspects, the antibody comprises a Kabat CDR-H2 sequence comprising, consisting of, or consisting essentially of SEQ ID NO:652. In some aspects, the antibody comprises a Kabat CDR-H2 sequence comprising, consisting of, or consisting essentially of SEQ ID NO:653. In some aspects, the antibody comprises a Kabat CDR-H2 sequence comprising, consisting of, or consisting essentially of SEQ ID NO:654. In some aspects, the antibody comprises a Kabat CDR-H2 sequence comprising, consisting of, or consisting essentially of SEQ ID NO:655. In some aspects, the antibody comprises a Kabat CDR-H2 sequence comprising, consisting of, or consisting essentially of SEQ ID NO:656. In some aspects, the antibody comprises a Kabat CDR-H2 sequence comprising, consisting of, or consisting essentially of SEQ ID NO:657. In some aspects, the antibody comprises a Kabat CDR-H2 sequence comprising, consisting of, or consisting essentially of SEQ ID NO: 658. In some aspects, the antibody comprises a Kabat CDR-H2 sequence comprising, consisting of, or consisting essentially of SEQ ID NO:659. In some aspects, the antibody comprises a Kabat CDR-H2 sequence comprising, consisting of, or consisting essentially of SEQ ID NO:660. In some aspects, the antibody comprises a Kabat CDR-H2 sequence comprising, consisting of, or consisting essentially of SEQ ID NO:661. In some aspects, the antibody comprises a Kabat CDR-H2 sequence comprising, consisting of, or consisting essentially of SEQ ID NO:662. In some aspects, the antibody comprises a Kabat CDR-H2 sequence comprising, consisting of, or consisting essentially of SEQ ID NO:663. In some aspects, the antibody comprises a Kabat CDR-H2 sequence comprising, consisting of, or consisting essentially of SEQ ID NO:664. In some aspects, the antibody comprises a Kabat CDR-H2 sequence comprising, consisting of, or consisting essentially of SEQ ID NO:665. In some aspects, the antibody comprises a Kabat CDR-H2 sequence comprising, consisting of, or consisting essentially of SEQ ID NO:666. In some aspects, the antibody comprises a Kabat CDR-H2 sequence comprising, consisting of, or consisting essentially of SEQ ID NO: 667. In some aspects, the antibody comprises a Kabat CDR-H2 sequence comprising, consisting of, or consisting essentially of SEQ ID NO:668. In some aspects, the antibody comprises a Kabat CDR-H2 sequence comprising, consisting of, or consisting essentially of SEQ ID NO:669. In some aspects, the antibody comprises a Kabat CDR-H2 sequence comprising, consisting of, or consisting essentially of SEQ ID NO:670. In some aspects, the antibody comprises a Kabat CDR-H2 sequence comprising, consisting of, or consisting essentially of SEQ ID NO:671.

### 5.2.1.3. Kabat CDR-H1

In some embodiments, the antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of a V_{H} sequence provided in SEQ ID NOs: 854-1020.

In some embodiments, the antibody comprises a V_{H} sequence comprising a Kabat CDR-H1 sequence comprising, consisting of, or consisting essentially of a sequence selected from SEQ ID NOs: 171-337. In some aspects, the antibody comprises a Kabat CDR-H1 sequence comprising, consisting of, or consisting essentially of SEQ ID NO:171. In some aspects, the antibody comprises a Kabat CDR-H1 sequence comprising, consisting of, or consisting essentially of SEQ ID NO:172. In some aspects, the antibody comprises a Kabat CDR-H1 sequence comprising, consisting of, or consisting essentially of SEQ ID NO: 173. In some aspects, the antibody comprises a Kabat CDR-H1 sequence comprising, consisting of, or consisting essentially of SEQ ID NO:174. In some aspects, the antibody comprises a Kabat CDR-H1 sequence comprising, consisting of, or consisting essentially of SEQ ID NO:175. In some aspects, the antibody comprises a Kabat CDR-H1 sequence comprising, consisting of, or consisting essentially of SEQ ID NO:176. In some aspects, the antibody comprises a Kabat CDR-H1 sequence comprising, consisting of, or consisting essentially of SEQ ID NO:177. In some aspects, the antibody comprises a Kabat CDR-H1 sequence comprising, consisting of, or consisting essentially of SEQ ID NO:178. In some aspects, the antibody comprises a Kabat CDR-H1 sequence comprising, consisting of, or consisting essentially of SEQ ID NO:179. In some aspects, the antibody comprises a Kabat CDR-H1 sequence comprising, consisting of, or consisting essentially of SEQ ID NO:180. In some aspects, the antibody comprises a Kabat CDR-H1 sequence comprising, consisting of, or consisting essentially of SEQ ID NO:181. In some aspects, the antibody comprises a Kabat CDR-H1 sequence comprising, consisting of, or consisting essentially of SEQ ID NO: 182. In some aspects, the antibody comprises a Kabat CDR-H1 sequence comprising, consisting of, or consisting essentially of SEQ ID NO:183. In some aspects, the antibody comprises a Kabat CDR-H1 sequence comprising, consisting of, or consisting essentially of SEQ ID NO:184. In some aspects, the antibody comprises a Kabat CDR-H1 sequence comprising, consisting of, or consisting essentially of SEQ ID NO:185. In some aspects, the antibody comprises a Kabat CDR-H1 sequence comprising, consisting of, or consisting essentially of SEQ ID NO:186. In some aspects, the antibody comprises a Kabat CDR-H1 sequence comprising, consisting of, or consisting essentially of SEQ ID NO:187. In some aspects, the antibody comprises a Kabat CDR-H1 sequence comprising, consisting of, or consisting essentially of SEQ ID NO:188. In some aspects, the antibody comprises a Kabat CDR-H1 sequence comprising, consisting of, or consisting essentially of SEQ ID NO:189. In some aspects, the antibody comprises a Kabat CDR-H1 sequence comprising, consisting of, or consisting essentially of SEQ ID NO:190. In some aspects, the antibody comprises a Kabat CDR-H1 sequence comprising, consisting of, or consisting essentially of SEQ ID NO: 191. In some aspects, the antibody comprises a Kabat CDR-H1 sequence comprising, consisting of, or consisting essentially of SEQ ID NO:192. In some aspects, the antibody comprises a Kabat CDR-H1 sequence comprising, consisting of, or consisting essentially of SEQ ID NO:193. In some aspects, the antibody comprises a Kabat CDR-H1 sequence comprising, consisting of, or consisting essentially of SEQ ID NO:194. In some aspects, the antibody comprises a Kabat CDR-H1 sequence comprising, consisting of, or consisting essentially of SEQ ID NO:195. In some aspects, the antibody comprises a Kabat CDR-H1 sequence comprising, consisting of, or consisting essentially of SEQ ID NO:196. In some aspects, the antibody comprises a Kabat CDR-H1 sequence comprising, consisting of, or consisting essentially of SEQ ID NO:197. In some aspects, the antibody comprises a Kabat CDR-H1 sequence comprising, consisting of, or consisting essentially of SEQ ID NO:198. In some aspects, the antibody comprises a Kabat CDR-H1 sequence comprising, consisting of, or consisting essentially of SEQ ID NO:199.

In some aspects, the antibody comprises a Kabat CDR-H1 sequence comprising, consisting of, or consisting essentially of SEQ ID NO:200. In some aspects, the antibody comprises a Kabat CDR-H1 sequence comprising, consisting of, or consisting essentially of SEQ ID NO:201. In some aspects, the antibody comprises a Kabat CDR-H1 sequence comprising, consisting of, or consisting essentially of SEQ ID NO:202. In some aspects, the antibody comprises a Kabat CDR-H1 sequence comprising, consisting of, or consisting essentially of SEQ ID NO:203. In some aspects, the antibody comprises a Kabat CDR-H1 sequence comprising, consisting of, or consisting essentially of SEQ ID NO:204. In some aspects, the antibody comprises a Kabat CDR-H1 sequence comprising, consisting of, or consisting essentially of SEQ ID NO:205. In some aspects, the antibody comprises a Kabat CDR-H1 sequence comprising, consisting of, or consisting essentially of SEQ ID NO:206. In some aspects, the antibody comprises a Kabat CDR-H1 sequence comprising, consisting of, or consisting essentially of SEQ ID NO:207. In some aspects, the antibody comprises a Kabat CDR-H1 sequence comprising, consisting of, or consisting essentially of SEQ ID NO: 208. In some aspects, the antibody comprises a Kabat CDR-H1 sequence comprising, consisting of, or consisting essentially of SEQ ID NO:209. In some aspects, the antibody comprises a Kabat CDR-H1 sequence comprising, consisting of, or consisting essentially of SEQ ID NO:210. In some aspects, the antibody comprises a Kabat CDR-H1 sequence comprising, consisting of, or consisting essentially of SEQ ID NO:211. In some aspects, the antibody comprises a Kabat CDR-H1 sequence comprising, consisting of, or consisting essentially of SEQ ID NO:212. In some aspects, the antibody comprises a Kabat CDR-H1 sequence comprising, consisting of, or consisting essentially of SEQ ID NO:213. In some aspects, the antibody comprises a Kabat CDR-H1 sequence comprising, consisting of, or consisting essentially of SEQ ID NO:214. In some aspects, the antibody comprises a Kabat CDR-H1 sequence comprising, consisting of, or consisting essentially of SEQ ID NO:215. In some aspects, the antibody comprises a Kabat CDR-H1 sequence comprising, consisting of, or consisting essentially of SEQ ID NO:216. In some aspects, the antibody comprises a Kabat CDR-H1 sequence comprising, consisting of, or consisting essentially of SEQ ID NO: 217. In some aspects, the antibody comprises a Kabat CDR-H1 sequence comprising, consisting of, or consisting essentially of SEQ ID NO:218. In some aspects, the antibody comprises a Kabat CDR-H1 sequence comprising, consisting of, or consisting essentially of SEQ ID NO:219. In some aspects, the antibody comprises a Kabat CDR-H1 sequence comprising, consisting of, or consisting essentially of SEQ ID NO:220. In some aspects, the antibody comprises a Kabat CDR-H1 sequence comprising, consisting of, or consisting essentially of SEQ ID NO:221. In some aspects, the antibody comprises a Kabat CDR-H1 sequence comprising, consisting of, or consisting essentially of SEQ ID NO:222. In some aspects, the antibody comprises a Kabat CDR-H1 sequence comprising, consisting of, or consisting essentially of SEQ ID NO:223. In some aspects, the antibody comprises a Kabat CDR-H1 sequence comprising, consisting of, or consisting essentially of SEQ ID NO:224. In some aspects, the antibody comprises a Kabat CDR-H1 sequence comprising, consisting of, or consisting essentially of SEQ ID NO:225. In some aspects, the antibody comprises a Kabat CDR-H1 sequence comprising, consisting of, or consisting essentially of SEQ ID NO: 226. In some aspects, the antibody comprises a Kabat CDR-H1 sequence comprising, consisting of, or consisting essentially of SEQ ID NO:227. In some aspects, the antibody comprises a Kabat CDR-H1 sequence comprising, consisting of, or consisting essentially of SEQ ID NO:228. In some aspects, the antibody comprises a Kabat CDR-H1 sequence comprising, consisting of, or consisting essentially of SEQ ID NO:229. In some aspects, the antibody comprises a Kabat CDR-H1 sequence comprising, consisting of, or consisting essentially of SEQ ID NO:230. In some aspects, the antibody comprises a Kabat CDR-H1 sequence comprising, consisting of, or consisting essentially of SEQ ID NO:231. In some aspects, the antibody comprises a Kabat CDR-H1 sequence comprising, consisting of, or consisting essentially of SEQ ID NO:232. In some aspects, the antibody comprises a Kabat CDR-H1 sequence comprising, consisting of, or consisting essentially of SEQ ID NO:233. In some aspects, the antibody comprises a Kabat CDR-H1 sequence comprising, consisting of, or consisting essentially of SEQ ID NO:234. In some aspects, the antibody comprises a Kabat CDR-H1 sequence comprising, consisting of, or consisting essentially of SEQ ID NO: 235. In some aspects, the antibody comprises a Kabat CDR-H1 sequence comprising, consisting of, or consisting essentially of SEQ ID NO:236. In some aspects, the antibody comprises a Kabat CDR-H1 sequence comprising, consisting of, or consisting essentially of SEQ ID NO:237. In some aspects, the antibody comprises a Kabat CDR-H1 sequence comprising, consisting of, or consisting essentially of SEQ ID NO:238. In some aspects, the antibody comprises a Kabat CDR-H1 sequence comprising, consisting of, or consisting essentially of SEQ ID NO:239. In some aspects, the antibody comprises a Kabat CDR-H1 sequence comprising, consisting of, or consisting essentially of SEQ ID NO:240. In some aspects, the antibody comprises a Kabat CDR-H1 sequence comprising, consisting of, or consisting essentially of SEQ ID NO:241. In some aspects, the antibody comprises a Kabat CDR-H1 sequence comprising, consisting of, or consisting essentially of SEQ ID NO:242. In some aspects, the antibody comprises a Kabat CDR-H1 sequence comprising, consisting of, or consisting essentially of SEQ ID NO:243. In some aspects, the antibody comprises a Kabat CDR-H1 sequence comprising, consisting of, or consisting essentially of SEQ ID NO: 244. In some aspects, the antibody comprises a Kabat CDR-H1 sequence comprising, consisting of, or consisting essentially of SEQ ID NO:245. In some aspects, the antibody comprises a Kabat CDR-H1 sequence comprising, consisting of, or consisting essentially of SEQ ID NO:246. In some aspects, the antibody comprises a Kabat CDR-H1 sequence comprising, consisting of, or consisting essentially of SEQ ID NO:247. In some aspects, the antibody comprises a Kabat CDR-H1 sequence comprising, consisting of, or consisting essentially of SEQ ID NO:248. In some aspects, the antibody comprises a Kabat CDR-H1 sequence comprising, consisting of, or consisting essentially of SEQ ID NO:249.

In some aspects, the antibody comprises a Kabat CDR-H1 sequence comprising, consisting of, or consisting essentially of SEQ ID NO:250. In some aspects, the antibody comprises a Kabat CDR-H1 sequence comprising, consisting of, or consisting essentially of SEQ ID NO:251. In some aspects, the antibody comprises a Kabat CDR-H1 sequence comprising, consisting of, or consisting essentially of SEQ ID NO:252. In some aspects, the antibody comprises a Kabat CDR-H1 sequence comprising, consisting of, or consisting essentially of SEQ ID NO:253. In some aspects, the antibody comprises a Kabat CDR-H1 sequence comprising, consisting of, or consisting essentially of SEQ ID NO:254. In some aspects, the antibody comprises a Kabat CDR-H1 sequence comprising, consisting of, or consisting essentially of SEQ ID NO:255. In some aspects, the antibody comprises a Kabat CDR-H1 sequence comprising, consisting of, or consisting essentially of SEQ ID NO:256. In some aspects, the antibody comprises a Kabat CDR-H1 sequence comprising, consisting of, or consisting essentially of SEQ ID NO:257. In some aspects, the antibody comprises a Kabat CDR-H1 sequence comprising, consisting of, or consisting essentially of SEQ ID NO: 258. In some aspects, the antibody comprises a Kabat CDR-H1 sequence comprising, consisting of, or consisting essentially of SEQ ID NO:259. In some aspects, the antibody comprises a Kabat CDR-H1 sequence comprising, consisting of, or consisting essentially of SEQ ID NO:260. In some aspects, the antibody comprises a Kabat CDR-H1 sequence comprising, consisting of, or consisting essentially of SEQ ID NO:261. In some aspects, the antibody comprises a Kabat CDR-H1 sequence comprising, consisting of, or consisting essentially of SEQ ID NO:262. In some aspects, the antibody comprises a Kabat CDR-H1 sequence comprising, consisting of, or consisting essentially of SEQ ID NO:263. In some aspects, the antibody comprises a Kabat CDR-H1 sequence comprising, consisting of, or consisting essentially of SEQ ID NO:264. In some aspects, the antibody comprises a Kabat CDR-H1 sequence comprising, consisting of, or consisting essentially of SEQ ID NO:265. In some aspects, the antibody comprises a Kabat CDR-H1 sequence comprising, consisting of, or consisting essentially of SEQ ID NO:266. In some aspects, the antibody comprises a Kabat CDR-H1 sequence comprising, consisting of, or consisting essentially of SEQ ID NO: 267. In some aspects, the antibody comprises a Kabat CDR-H1 sequence comprising, consisting of, or consisting essentially of SEQ ID NO:268. In some aspects, the antibody comprises a Kabat CDR-H1 sequence comprising, consisting of, or consisting essentially of SEQ ID NO:269. In some aspects, the antibody comprises a Kabat CDR-H1 sequence comprising, consisting of, or consisting essentially of SEQ ID NO:270. In some aspects, the antibody comprises a Kabat CDR-H1 sequence comprising, consisting of, or consisting essentially of SEQ ID NO:271. In some aspects, the antibody comprises a Kabat CDR-H1 sequence comprising, consisting of, or consisting essentially of SEQ ID NO:272. In some aspects, the antibody comprises a Kabat CDR-H1 sequence comprising, consisting of, or consisting essentially of SEQ ID NO:273. In some aspects, the antibody comprises a Kabat CDR-H1 sequence comprising, consisting of, or consisting essentially of SEQ ID NO:274. In some aspects, the antibody comprises a Kabat CDR-H1 sequence comprising, consisting of, or consisting essentially of SEQ ID NO:275. In some aspects, the antibody comprises a Kabat CDR-H1 sequence comprising, consisting of, or consisting essentially of SEQ ID NO: 276. In some aspects, the antibody comprises a Kabat CDR-H1 sequence comprising, consisting of, or consisting essentially of SEQ ID NO:277. In some aspects, the antibody comprises a Kabat CDR-H1 sequence comprising, consisting of, or consisting essentially of SEQ ID NO:278. In some aspects, the antibody comprises a Kabat CDR-H1 sequence comprising, consisting of, or consisting essentially of SEQ ID NO:279. In some aspects, the antibody comprises a Kabat CDR-H1 sequence comprising, consisting of, or consisting essentially of SEQ ID NO:280. In some aspects, the antibody comprises a Kabat CDR-H1 sequence comprising, consisting of, or consisting essentially of SEQ ID NO:281. In some aspects, the antibody comprises a Kabat CDR-H1 sequence comprising, consisting of, or consisting essentially of SEQ ID NO:282. In some aspects, the antibody comprises a Kabat CDR-H1 sequence comprising, consisting of, or consisting essentially of SEQ ID NO:283. In some aspects, the antibody comprises a Kabat CDR-H1 sequence comprising, consisting of, or consisting essentially of SEQ ID NO:284. In some aspects, the antibody comprises a Kabat CDR-H1 sequence comprising, consisting of, or consisting essentially of SEQ ID NO: 285. In some aspects, the antibody comprises a Kabat CDR-H1 sequence comprising, consisting of, or consisting essentially of SEQ ID NO:286. In some aspects, the antibody comprises a Kabat CDR-H1 sequence comprising, consisting of, or consisting essentially of SEQ ID NO:287. In some aspects, the antibody comprises a Kabat CDR-H1 sequence comprising, consisting of, or consisting essentially of SEQ ID NO:288. In some aspects, the antibody comprises a Kabat CDR-H1 sequence comprising, consisting of, or consisting essentially of SEQ ID NO:289. In some aspects, the antibody comprises a Kabat CDR-H1 sequence comprising, consisting of, or consisting essentially of SEQ ID NO:290. In some aspects, the antibody comprises a Kabat CDR-H1 sequence comprising, consisting of, or consisting essentially of SEQ ID NO:291. In some aspects, the antibody comprises a Kabat CDR-H1 sequence comprising, consisting of, or consisting essentially of SEQ ID NO:292. In some aspects, the antibody comprises a Kabat CDR-H1 sequence comprising, consisting of, or consisting essentially of SEQ ID NO:293. In some aspects, the antibody comprises a Kabat CDR-H1 sequence comprising, consisting of, or consisting essentially of SEQ ID NO: 294. In some aspects, the antibody comprises a Kabat CDR-H1 sequence comprising, consisting of, or consisting essentially of SEQ ID NO:295. In some aspects, the antibody comprises a Kabat CDR-H1 sequence comprising, consisting of, or consisting essentially of SEQ ID NO:296. In some aspects, the antibody comprises a Kabat CDR-H1 sequence comprising, consisting of, or consisting essentially of SEQ ID NO:297. In some aspects, the antibody comprises a Kabat CDR-H1 sequence comprising, consisting of, or consisting essentially of SEQ ID NO:298. In some aspects, the antibody comprises a Kabat CDR-H1 sequence comprising, consisting of, or consisting essentially of SEQ ID NO:299.

In some aspects, the antibody comprises a Kabat CDR-H1 sequence comprising, consisting of, or consisting essentially of SEQ ID NO:300. In some aspects, the antibody comprises a Kabat CDR-H1 sequence comprising, consisting of, or consisting essentially of SEQ ID NO:301. In some aspects, the antibody comprises a Kabat CDR-H1 sequence comprising, consisting of, or consisting essentially of SEQ ID NO:302. In some aspects, the antibody comprises a Kabat CDR-H1 sequence comprising, consisting of, or consisting essentially of SEQ ID NO:303. In some aspects, the antibody comprises a Kabat CDR-H1 sequence comprising, consisting of, or consisting essentially of SEQ ID NO:304. In some aspects, the antibody comprises a Kabat CDR-H1 sequence comprising, consisting of, or consisting essentially of SEQ ID NO:305. In some aspects, the antibody comprises a Kabat CDR-H1 sequence comprising, consisting of, or consisting essentially of SEQ ID NO:306. In some aspects, the antibody comprises a Kabat CDR-H1 sequence comprising, consisting of, or consisting essentially of SEQ ID NO:307. In some aspects, the antibody comprises a Kabat CDR-H1 sequence comprising, consisting of, or consisting essentially of SEQ ID NO: 308. In some aspects, the antibody comprises a Kabat CDR-H1 sequence comprising, consisting of, or consisting essentially of SEQ ID NO:309. In some aspects, the antibody comprises a Kabat CDR-H1 sequence comprising, consisting of, or consisting essentially of SEQ ID NO:310. In some aspects, the antibody comprises a Kabat CDR-H1 sequence comprising, consisting of, or consisting essentially of SEQ ID NO:311. In some aspects, the antibody comprises a Kabat CDR-H1 sequence comprising, consisting of, or consisting essentially of SEQ ID NO:312. In some aspects, the antibody comprises a Kabat CDR-H1 sequence comprising, consisting of, or consisting essentially of SEQ ID NO:313. In some aspects, the antibody comprises a Kabat CDR-H1 sequence comprising, consisting of, or consisting essentially of SEQ ID NO:314. In some aspects, the antibody comprises a Kabat CDR-H1 sequence comprising, consisting of, or consisting essentially of SEQ ID NO:315. In some aspects, the antibody comprises a Kabat CDR-H1 sequence comprising, consisting of, or consisting essentially of SEQ ID NO:316. In some aspects, the antibody comprises a Kabat CDR-H1 sequence comprising, consisting of, or consisting essentially of SEQ ID NO: 317. In some aspects, the antibody comprises a Kabat CDR-H1 sequence comprising, consisting of, or consisting essentially of SEQ ID NO:318. In some aspects, the antibody comprises a Kabat CDR-H1 sequence comprising, consisting of, or consisting essentially of SEQ ID NO:319. In some aspects, the antibody comprises a Kabat CDR-H1 sequence comprising, consisting of, or consisting essentially of SEQ ID NO:320. In some aspects, the antibody comprises a Kabat CDR-H1 sequence comprising, consisting of, or consisting essentially of SEQ ID NO:321. In some aspects, the antibody comprises a Kabat CDR-H1 sequence comprising, consisting of, or consisting essentially of SEQ ID NO:322. In some aspects, the antibody comprises a Kabat CDR-H1 sequence comprising, consisting of, or consisting essentially of SEQ ID NO:323. In some aspects, the antibody comprises a Kabat CDR-H1 sequence comprising, consisting of, or consisting essentially of SEQ ID NO:324. In some aspects, the antibody comprises a Kabat CDR-H1 sequence comprising, consisting of, or consisting essentially of SEQ ID NO:325. In some aspects, the antibody comprises a Kabat CDR-H1 sequence comprising, consisting of, or consisting essentially of SEQ ID NO: 326. In some aspects, the antibody comprises a Kabat CDR-H1 sequence comprising, consisting of, or consisting essentially of SEQ ID NO:327. In some aspects, the antibody comprises a Kabat CDR-H1 sequence comprising, consisting of, or consisting essentially of SEQ ID NO:328. In some aspects, the antibody comprises a Kabat CDR-H1 sequence comprising, consisting of, or consisting essentially of SEQ ID NO:329. In some aspects, the antibody comprises a Kabat CDR-H1 sequence comprising, consisting of, or consisting essentially of SEQ ID NO:330. In some aspects, the antibody comprises a Kabat CDR-H1 sequence comprising, consisting of, or consisting essentially of SEQ ID NO:331. In some aspects, the antibody comprises a Kabat CDR-H1 sequence comprising, consisting of, or consisting essentially of SEQ ID NO:332. In some aspects, the antibody comprises a Kabat CDR-H1 sequence comprising, consisting of, or consisting essentially of SEQ ID NO:333. In some aspects, the antibody comprises a Kabat CDR-H1 sequence comprising, consisting of, or consisting essentially of SEQ ID NO:334. In some aspects, the antibody comprises a Kabat CDR-H1 sequence comprising, consisting of, or consisting essentially of SEQ ID NO: 335. In some aspects, the antibody comprises a Kabat CDR-H1 sequence comprising, consisting of, or consisting essentially of SEQ ID NO:336. In some aspects, the antibody comprises a Kabat CDR-H1 sequence comprising, consisting of, or consisting essentially of SEQ ID NO:337.

### 5.2.1.4. Kabat CDR-H3+Kabat CDR-H2

In some embodiments, the antibody comprises a V_{H} sequence comprising a Kabat CDR-H3 sequence comprising, consisting of, or consisting essentially of a sequence selected from SEQ ID NOs: 672-838, and a Kabat CDR-H2 sequence comprising, consisting of, or consisting essentially of a sequence selected from SEQ ID NOs: 505-671. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 854-1020.

### 5.2.1.5. Kabat CDR-H3+Kabat CDR-H1

In some embodiments, the antibody comprises a V_{H} sequence comprising a Kabat CDR-H3 sequence comprising, consisting of, or consisting essentially of a sequence selected from SEQ ID NOs: 672-838, and a Kabat CDR-H1 sequence comprising, consisting of, or consisting essentially of a sequence selected from SEQ ID NOs: 171-337. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 854-1020.

### 5.2.1.6. Kabat CDR-H1+Kabat CDR-H2

In some embodiments, the antibody comprises a V_{H} sequence comprising a Kabat CDR-H1 sequence comprising, consisting of, or consisting essentially of a sequence selected from SEQ ID NOs: 171-337 and a Kabat CDR-H2 sequence comprising, consisting of, or consisting essentially of a sequence selected from SEQ ID NOs: 505-671. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 854-1020.

### 5.2.1.7. Kabat CDR-H1+Kabat CDR-H2+Kabat CDR-H3

In some embodiments, the antibody comprises a V_{H} sequence comprising a Kabat CDR-H1 sequence comprising, consisting of, or consisting essentially of a sequence selected from SEQ ID NOs: 171-337, a Kabat CDR-H2 sequence comprising, consisting of, or consisting essentially of a sequence selected from SEQ ID NOs: 505-671, and a Kabat CDR-H3 sequence comprising, consisting of, or consisting essentially of a sequence selected from SEQ ID NOs: 672-838. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 854-1020.

### 5.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, 95%, 96%, 97%, 98%, or 99% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, 95%, 96%, 97%, 98%, or 99% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, 95%, 96%, 97%, 98%, or 99% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

### 5.2.2. V_{H} Sequences Comprising Illustrative Chothia CDRs

In some embodiments, the antibody comprises a V_{H} sequence comprising one or more Chothia CDR-H sequences comprising, consisting of, or consisting essentially of one or more illustrative Chothia CDR-H sequences provided in this disclosure, and variants thereof.

### 5.2.2.1. Chothia CDR-H3

In some embodiments, the antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of a V_{H} sequence provided in SEQ ID NOs: 854-1020.

In some embodiments, the antibody comprises a V_{H} sequence comprising a Chothia CDR-H3 sequence comprising, consisting of, or consisting essentially of a sequence selected from SEQ ID NOs: 672-838. In some aspects, the antibody comprises a Chothia CDR-H3 sequence comprising, consisting of, or consisting essentially of SEQ ID NO: 672. In some aspects, the antibody comprises a Chothia CDR-H3 sequence comprising, consisting of, or consisting essentially of SEQ ID NO:673. In some aspects, the antibody comprises a Chothia CDR-H3 sequence comprising, consisting of, or consisting essentially of SEQ ID NO:674. In some aspects, the antibody comprises a Chothia CDR-H3 sequence comprising, consisting of, or consisting essentially of SEQ ID NO:675. In some aspects, the antibody comprises a Chothia CDR-H3 sequence comprising, consisting of, or consisting essentially of SEQ ID NO:676. In some aspects, the antibody comprises a Chothia CDR-H3 sequence comprising, consisting of, or consisting essentially of SEQ ID NO: 677. In some aspects, the antibody comprises a Chothia CDR-H3 sequence comprising, consisting of, or consisting essentially of SEQ ID NO:678. In some aspects, the antibody comprises a Chothia CDR-H3 sequence comprising, consisting of, or consisting essentially of SEQ ID NO:679. In some aspects, the antibody comprises a Chothia CDR-H3 sequence comprising, consisting of, or consisting essentially of SEQ ID NO:680. In some aspects, the antibody comprises a Chothia CDR-H3 sequence comprising, consisting of, or consisting essentially of SEQ ID NO:681. In some aspects, the antibody comprises a Chothia CDR-H3 sequence comprising, consisting of, or consisting essentially of SEQ ID NO: 682. In some aspects, the antibody comprises a Chothia CDR-H3 sequence comprising, consisting of, or consisting essentially of SEQ ID NO:683. In some aspects, the antibody comprises a Chothia CDR-H3 sequence comprising, consisting of, or consisting essentially of SEQ ID NO:684. In some aspects, the antibody comprises a Chothia CDR-H3 sequence comprising, consisting of, or consisting essentially of SEQ ID NO:685. In some aspects, the antibody comprises a Chothia CDR-H3 sequence comprising, consisting of, or consisting essentially of SEQ ID NO:686. In some aspects, the antibody comprises a Chothia CDR-H3 sequence comprising, consisting of, or consisting essentially of SEQ ID NO: 687. In some aspects, the antibody comprises a Chothia CDR-H3 sequence comprising, consisting of, or consisting essentially of SEQ ID NO:688. In some aspects, the antibody comprises a Chothia CDR-H3 sequence comprising, consisting of, or consisting essentially of SEQ ID NO:689. In some aspects, the antibody comprises a Chothia CDR-H3 sequence comprising, consisting of, or consisting essentially of SEQ ID NO:690. In some aspects, the antibody comprises a Chothia CDR-H3 sequence comprising, consisting of, or consisting essentially of SEQ ID NO:691. In some aspects, the antibody comprises a Chothia CDR-H3 sequence comprising, consisting of, or consisting essentially of SEQ ID NO: 692. In some aspects, the antibody comprises a Chothia CDR-H3 sequence comprising, consisting of, or consisting essentially of SEQ ID NO:693. In some aspects, the antibody comprises a Chothia CDR-H3 sequence comprising, consisting of, or consisting essentially of SEQ ID NO:694. In some aspects, the antibody comprises a Chothia CDR-H3 sequence comprising, consisting of, or consisting essentially of SEQ ID NO:695. In some aspects, the antibody comprises a Chothia CDR-H3 sequence comprising, consisting of, or consisting essentially of SEQ ID NO:696. In some aspects, the antibody comprises a Chothia CDR-H3 sequence comprising, consisting of, or consisting essentially of SEQ ID NO: 697. In some aspects, the antibody comprises a Chothia CDR-H3 sequence comprising, consisting of, or consisting essentially of SEQ ID NO:698. In some aspects, the antibody comprises a Chothia CDR-H3 sequence comprising, consisting of, or consisting essentially of SEQ ID NO:699.

In some aspects, the antibody comprises a Chothia CDR-H3 sequence comprising, consisting of, or consisting essentially of SEQ ID NO:700. In some aspects, the antibody comprises a Chothia CDR-H3 sequence comprising, consisting of, or consisting essentially of SEQ ID NO:701. In some aspects, the antibody comprises a Chothia CDR-H3 sequence comprising, consisting of, or consisting essentially of SEQ ID NO:702. In some aspects, the antibody comprises a Chothia CDR-H3 sequence comprising, consisting of, or consisting essentially of SEQ ID NO:703. In some aspects, the antibody comprises a Chothia CDR-H3 sequence comprising, consisting of, or consisting essentially of SEQ ID NO: 704. In some aspects, the antibody comprises a Chothia CDR-H3 sequence comprising, consisting of, or consisting essentially of SEQ ID NO:705. In some aspects, the antibody comprises a Chothia CDR-H3 sequence comprising, consisting of, or consisting essentially of SEQ ID NO:706. In some aspects, the antibody comprises a Chothia CDR-H3 sequence comprising, consisting of, or consisting essentially of SEQ ID NO:707. In some aspects, the antibody comprises a Chothia CDR-H3 sequence comprising, consisting of, or consisting essentially of SEQ ID NO:708. In some aspects, the antibody comprises a Chothia CDR-H3 sequence comprising, consisting of, or consisting essentially of SEQ ID NO: 709. In some aspects, the antibody comprises a Chothia CDR-H3 sequence comprising, consisting of, or consisting essentially of SEQ ID NO:710. In some aspects, the antibody comprises a Chothia CDR-H3 sequence comprising, consisting of, or consisting essentially of SEQ ID NO:711. In some aspects, the antibody comprises a Chothia CDR-H3 sequence comprising, consisting of, or consisting essentially of SEQ ID NO:712. In some aspects, the antibody comprises a Chothia CDR-H3 sequence comprising, consisting of, or consisting essentially of SEQ ID NO:713. In some aspects, the antibody comprises a Chothia CDR-H3 sequence comprising, consisting of, or consisting essentially of SEQ ID NO: 714. In some aspects, the antibody comprises a Chothia CDR-H3 sequence comprising, consisting of, or consisting essentially of SEQ ID NO:715. In some aspects, the antibody comprises a Chothia CDR-H3 sequence comprising, consisting of, or consisting essentially of SEQ ID NO:716. In some aspects, the antibody comprises a Chothia CDR-H3 sequence comprising, consisting of, or consisting essentially of SEQ ID NO:717. In some aspects, the antibody comprises a Chothia CDR-H3 sequence comprising, consisting of, or consisting essentially of SEQ ID NO:718. In some aspects, the antibody comprises a Chothia CDR-H3 sequence comprising, consisting of, or consisting essentially of SEQ ID NO: 719. In some aspects, the antibody comprises a Chothia CDR-H3 sequence comprising, consisting of, or consisting essentially of SEQ ID NO:720. In some aspects, the antibody comprises a Chothia CDR-H3 sequence comprising, consisting of, or consisting essentially of SEQ ID NO:721. In some aspects, the antibody comprises a Chothia CDR-H3 sequence comprising, consisting of, or consisting essentially of SEQ ID NO:722. In some aspects, the antibody comprises a Chothia CDR-H3 sequence comprising, consisting of, or consisting essentially of SEQ ID NO:723. In some aspects, the antibody comprises a Chothia CDR-H3 sequence comprising, consisting of, or consisting essentially of SEQ ID NO: 724. In some aspects, the antibody comprises a Chothia CDR-H3 sequence comprising, consisting of, or consisting essentially of SEQ ID NO:725. In some aspects, the antibody comprises a Chothia CDR-H3 sequence comprising, consisting of, or consisting essentially of SEQ ID NO:726. In some aspects, the antibody comprises a Chothia CDR-H3 sequence comprising, consisting of, or consisting essentially of SEQ ID NO:727. In some aspects, the antibody comprises a Chothia CDR-H3 sequence comprising, consisting of, or consisting essentially of SEQ ID NO:728. In some aspects, the antibody comprises a Chothia CDR-H3 sequence comprising, consisting of, or consisting essentially of SEQ ID NO: 729. In some aspects, the antibody comprises a Chothia CDR-H3 sequence comprising, consisting of, or consisting essentially of SEQ ID NO:730. In some aspects, the antibody comprises a Chothia CDR-H3 sequence comprising, consisting of, or consisting essentially of SEQ ID NO:731. In some aspects, the antibody comprises a Chothia CDR-H3 sequence comprising, consisting of, or consisting essentially of SEQ ID NO:732. In some aspects, the antibody comprises a Chothia CDR-H3 sequence comprising, consisting of, or consisting essentially of SEQ ID NO:733. In some aspects, the antibody comprises a Chothia CDR-H3 sequence comprising, consisting of, or consisting essentially of SEQ ID NO: 734. In some aspects, the antibody comprises a Chothia CDR-H3 sequence comprising, consisting of, or consisting essentially of SEQ ID NO:735. In some aspects, the antibody comprises a Chothia CDR-H3 sequence comprising, consisting of, or consisting essentially of SEQ ID NO:736. In some aspects, the antibody comprises a Chothia CDR-H3 sequence comprising, consisting of, or consisting essentially of SEQ ID NO:737. In some aspects, the antibody comprises a Chothia CDR-H3 sequence comprising, consisting of, or consisting essentially of SEQ ID NO:738. In some aspects, the antibody comprises a Chothia CDR-H3 sequence comprising, consisting of, or consisting essentially of SEQ ID NO: 739. In some aspects, the antibody comprises a Chothia CDR-H3 sequence comprising, consisting of, or consisting essentially of SEQ ID NO:740. In some aspects, the antibody comprises a Chothia CDR-H3 sequence comprising, consisting of, or consisting essentially of SEQ ID NO:741. In some aspects, the antibody comprises a Chothia CDR-H3 sequence comprising, consisting of, or consisting essentially of SEQ ID NO:742. In some aspects, the antibody comprises a Chothia CDR-H3 sequence comprising, consisting of, or consisting essentially of SEQ ID NO:743. In some aspects, the antibody comprises a Chothia CDR-H3 sequence comprising, consisting of, or consisting essentially of SEQ ID NO: 744. In some aspects, the antibody comprises a Chothia CDR-H3 sequence comprising, consisting of, or consisting essentially of SEQ ID NO:745. In some aspects, the antibody comprises a Chothia CDR-H3 sequence comprising, consisting of, or consisting essentially of SEQ ID NO:746. In some aspects, the antibody comprises a Chothia CDR-H3 sequence comprising, consisting of, or consisting essentially of SEQ ID NO:747. In some aspects, the antibody comprises a Chothia CDR-H3 sequence comprising, consisting of, or consisting essentially of SEQ ID NO:748. In some aspects, the antibody comprises a Chothia CDR-H3 sequence comprising, consisting of, or consisting essentially of SEQ ID NO: 749.

In some aspects, the antibody comprises a Chothia CDR-H3 sequence comprising, consisting of, or consisting essentially of SEQ ID NO:750. In some aspects, the antibody comprises a Chothia CDR-H3 sequence comprising, consisting of, or consisting essentially of SEQ ID NO:751. In some aspects, the antibody comprises a Chothia CDR-H3 sequence comprising, consisting of, or consisting essentially of SEQ ID NO:752. In some aspects, the antibody comprises a Chothia CDR-H3 sequence comprising, consisting of, or consisting essentially of SEQ ID NO:753. In some aspects, the antibody comprises a Chothia CDR-H3 sequence comprising, consisting of, or consisting essentially of SEQ ID NO: 754. In some aspects, the antibody comprises a Chothia CDR-H3 sequence comprising, consisting of, or consisting essentially of SEQ ID NO:755. In some aspects, the antibody comprises a Chothia CDR-H3 sequence comprising, consisting of, or consisting essentially of SEQ ID NO:756. In some aspects, the antibody comprises a Chothia CDR-H3 sequence comprising, consisting of, or consisting essentially of SEQ ID NO:757. In some aspects, the antibody comprises a Chothia CDR-H3 sequence comprising, consisting of, or consisting essentially of SEQ ID NO:758. In some aspects, the antibody comprises a Chothia CDR-H3 sequence comprising, consisting of, or consisting essentially of SEQ ID NO: 759. In some aspects, the antibody comprises a Chothia CDR-H3 sequence comprising, consisting of, or consisting essentially of SEQ ID NO:760. In some aspects, the antibody comprises a Chothia CDR-H3 sequence comprising, consisting of, or consisting essentially of SEQ ID NO:761. In some aspects, the antibody comprises a Chothia CDR-H3 sequence comprising, consisting of, or consisting essentially of SEQ ID NO:762. In some aspects, the antibody comprises a Chothia CDR-H3 sequence comprising, consisting of, or consisting essentially of SEQ ID NO:763. In some aspects, the antibody comprises a Chothia CDR-H3 sequence comprising, consisting of, or consisting essentially of SEQ ID NO: 764. In some aspects, the antibody comprises a Chothia CDR-H3 sequence comprising, consisting of, or consisting essentially of SEQ ID NO:765. In some aspects, the antibody comprises a Chothia CDR-H3 sequence comprising, consisting of, or consisting essentially of SEQ ID NO:766. In some aspects, the antibody comprises a Chothia CDR-H3 sequence comprising, consisting of, or consisting essentially of SEQ ID NO:767. In some aspects, the antibody comprises a Chothia CDR-H3 sequence comprising, consisting of, or consisting essentially of SEQ ID NO:768. In some aspects, the antibody comprises a Chothia CDR-H3 sequence comprising, consisting of, or consisting essentially of SEQ ID NO: 769. In some aspects, the antibody comprises a Chothia CDR-H3 sequence comprising, consisting of, or consisting essentially of SEQ ID NO:770. In some aspects, the antibody comprises a Chothia CDR-H3 sequence comprising, consisting of, or consisting essentially of SEQ ID NO:771. In some aspects, the antibody comprises a Chothia CDR-H3 sequence comprising, consisting of, or consisting essentially of SEQ ID NO:772. In some aspects, the antibody comprises a Chothia CDR-H3 sequence comprising, consisting of, or consisting essentially of SEQ ID NO:773. In some aspects, the antibody comprises a Chothia CDR-H3 sequence comprising, consisting of, or consisting essentially of SEQ ID NO: 774. In some aspects, the antibody comprises a Chothia CDR-H3 sequence comprising, consisting of, or consisting essentially of SEQ ID NO:775. In some aspects, the antibody comprises a Chothia CDR-H3 sequence comprising, consisting of, or consisting essentially of SEQ ID NO:776. In some aspects, the antibody comprises a Chothia CDR-H3 sequence comprising, consisting of, or consisting essentially of SEQ ID NO:777. In some aspects, the antibody comprises a Chothia CDR-H3 sequence comprising, consisting of, or consisting essentially of SEQ ID NO:778. In some aspects, the antibody comprises a Chothia CDR-H3 sequence comprising, consisting of, or consisting essentially of SEQ ID NO: 779. In some aspects, the antibody comprises a Chothia CDR-H3 sequence comprising, consisting of, or consisting essentially of SEQ ID NO:780. In some aspects, the antibody comprises a Chothia CDR-H3 sequence comprising, consisting of, or consisting essentially of SEQ ID NO:781. In some aspects, the antibody comprises a Chothia CDR-H3 sequence comprising, consisting of, or consisting essentially of SEQ ID NO:782. In some aspects, the antibody comprises a Chothia CDR-H3 sequence comprising, consisting of, or consisting essentially of SEQ ID NO:783. In some aspects, the antibody comprises a Chothia CDR-H3 sequence comprising, consisting of, or consisting essentially of SEQ ID NO: 784. In some aspects, the antibody comprises a Chothia CDR-H3 sequence comprising, consisting of, or consisting essentially of SEQ ID NO:785. In some aspects, the antibody comprises a Chothia CDR-H3 sequence comprising, consisting of, or consisting essentially of SEQ ID NO:786. In some aspects, the antibody comprises a Chothia CDR-H3 sequence comprising, consisting of, or consisting essentially of SEQ ID NO:787. In some aspects, the antibody comprises a Chothia CDR-H3 sequence comprising, consisting of, or consisting essentially of SEQ ID NO:788. In some aspects, the antibody comprises a Chothia CDR-H3 sequence comprising, consisting of, or consisting essentially of SEQ ID NO: 789. In some aspects, the antibody comprises a Chothia CDR-H3 sequence comprising, consisting of, or consisting essentially of SEQ ID NO:790. In some aspects, the antibody comprises a Chothia CDR-H3 sequence comprising, consisting of, or consisting essentially of SEQ ID NO:791. In some aspects, the antibody comprises a Chothia CDR-H3 sequence comprising, consisting of, or consisting essentially of SEQ ID NO:792. In some aspects, the antibody comprises a Chothia CDR-H3 sequence comprising, consisting of, or consisting essentially of SEQ ID NO:793. In some aspects, the antibody comprises a Chothia CDR-H3 sequence comprising, consisting of, or consisting essentially of SEQ ID NO: 794. In some aspects, the antibody comprises a Chothia CDR-H3 sequence comprising, consisting of, or consisting essentially of SEQ ID NO:795. In some aspects, the antibody comprises a Chothia CDR-H3 sequence comprising, consisting of, or consisting essentially of SEQ ID NO:796. In some aspects, the antibody comprises a Chothia CDR-H3 sequence comprising, consisting of, or consisting essentially of SEQ ID NO:797. In some aspects, the antibody comprises a Chothia CDR-H3 sequence comprising, consisting of, or consisting essentially of SEQ ID NO:798. In some aspects, the antibody comprises a Chothia CDR-H3 sequence comprising, consisting of, or consisting essentially of SEQ ID NO: 799.

In some aspects, the antibody comprises a Chothia CDR-H3 sequence comprising, consisting of, or consisting essentially of SEQ ID NO:800. In some aspects, the antibody comprises a Chothia CDR-H3 sequence comprising, consisting of, or consisting essentially of SEQ ID NO:801. In some aspects, the antibody comprises a Chothia CDR-H3 sequence comprising, consisting of, or consisting essentially of SEQ ID NO:802. In some aspects, the antibody comprises a Chothia CDR-H3 sequence comprising, consisting of, or consisting essentially of SEQ ID NO:803. In some aspects, the antibody comprises a Chothia CDR-H3 sequence comprising, consisting of, or consisting essentially of SEQ ID NO: 804. In some aspects, the antibody comprises a Chothia CDR-H3 sequence comprising, consisting of, or consisting essentially of SEQ ID NO:805. In some aspects, the antibody comprises a Chothia CDR-H3 sequence comprising, consisting of, or consisting essentially of SEQ ID NO:806. In some aspects, the antibody comprises a Chothia CDR-H3 sequence comprising, consisting of, or consisting essentially of SEQ ID NO:807. In some aspects, the antibody comprises a Chothia CDR-H3 sequence comprising, consisting of, or consisting essentially of SEQ ID NO:808. In some aspects, the antibody comprises a Chothia CDR-H3 sequence comprising, consisting of, or consisting essentially of SEQ ID NO: 809. In some aspects, the antibody comprises a Chothia CDR-H3 sequence comprising, consisting of, or consisting essentially of SEQ ID NO:810. In some aspects, the antibody comprises a Chothia CDR-H3 sequence comprising, consisting of, or consisting essentially of SEQ ID NO:811. In some aspects, the antibody comprises a Chothia CDR-H3 sequence comprising, consisting of, or consisting essentially of SEQ ID NO:812. In some aspects, the antibody comprises a Chothia CDR-H3 sequence comprising, consisting of, or consisting essentially of SEQ ID NO:813. In some aspects, the antibody comprises a Chothia CDR-H3 sequence comprising, consisting of, or consisting essentially of SEQ ID NO: 814. In some aspects, the antibody comprises a Chothia CDR-H3 sequence comprising, consisting of, or consisting essentially of SEQ ID NO:815. In some aspects, the antibody comprises a Chothia CDR-H3 sequence comprising, consisting of, or consisting essentially of SEQ ID NO:816. In some aspects, the antibody comprises a Chothia CDR-H3 sequence comprising, consisting of, or consisting essentially of SEQ ID NO:817. In some aspects, the antibody comprises a Chothia CDR-H3 sequence comprising, consisting of, or consisting essentially of SEQ ID NO:818. In some aspects, the antibody comprises a Chothia CDR-H3 sequence comprising, consisting of, or consisting essentially of SEQ ID NO: 819. In some aspects, the antibody comprises a Chothia CDR-H3 sequence comprising, consisting of, or consisting essentially of SEQ ID NO:820. In some aspects, the antibody comprises a Chothia CDR-H3 sequence comprising, consisting of, or consisting essentially of SEQ ID NO:821. In some aspects, the antibody comprises a Chothia CDR-H3 sequence comprising, consisting of, or consisting essentially of SEQ ID NO:822. In some aspects, the antibody comprises a Chothia CDR-H3 sequence comprising, consisting of, or consisting essentially of SEQ ID NO:823. In some aspects, the antibody comprises a Chothia CDR-H3 sequence comprising, consisting of, or consisting essentially of SEQ ID NO: 824. In some aspects, the antibody comprises a Chothia CDR-H3 sequence comprising, consisting of, or consisting essentially of SEQ ID NO:825. In some aspects, the antibody comprises a Chothia CDR-H3 sequence comprising, consisting of, or consisting essentially of SEQ ID NO:826. In some aspects, the antibody comprises a Chothia CDR-H3 sequence comprising, consisting of, or consisting essentially of SEQ ID NO:827. In some aspects, the antibody comprises a Chothia CDR-H3 sequence comprising, consisting of, or consisting essentially of SEQ ID NO:828. In some aspects, the antibody comprises a Chothia CDR-H3 sequence comprising, consisting of, or consisting essentially of SEQ ID NO: 829. In some aspects, the antibody comprises a Chothia CDR-H3 sequence comprising, consisting of, or consisting essentially of SEQ ID NO:830. In some aspects, the antibody comprises a Chothia CDR-H3 sequence comprising, consisting of, or consisting essentially of SEQ ID NO:831. In some aspects, the antibody comprises a Chothia CDR-H3 sequence comprising, consisting of, or consisting essentially of SEQ ID NO:832. In some aspects, the antibody comprises a Chothia CDR-H3 sequence comprising, consisting of, or consisting essentially of SEQ ID NO:833. In some aspects, the antibody comprises a Chothia CDR-H3 sequence comprising, consisting of, or consisting essentially of SEQ ID NO: 834. In some aspects, the antibody comprises a Chothia CDR-H3 sequence comprising, consisting of, or consisting essentially of SEQ ID NO:835. In some aspects, the antibody comprises a Chothia CDR-H3 sequence comprising, consisting of, or consisting essentially of SEQ ID NO:836. In some aspects, the antibody comprises a Chothia CDR-H3 sequence comprising, consisting of, or consisting essentially of SEQ ID NO:837. In some aspects, the antibody comprises a Chothia CDR-H3 sequence comprising, consisting of, or consisting essentially of SEQ ID NO:838.

### 5.2.2.2. Chothia CDR-H2

In some embodiments, the antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of a V_{H} sequence provided in SEQ ID NOs: 854-1020.

In some embodiments, the antibody comprises a V_{H} sequence comprising a Chothia CDR-H2 sequence comprising, consisting of, or consisting essentially of a sequence selected from SEQ ID NOs: 338-504. In some aspects, the antibody comprises a Chothia CDR-H2 sequence comprising, consisting of, or consisting essentially of SEQ ID NO: 338. In some aspects, the antibody comprises a Chothia CDR-H2 sequence comprising, consisting of, or consisting essentially of SEQ ID NO:339. In some aspects, the antibody comprises a Chothia CDR-H2 sequence comprising, consisting of, or consisting essentially of SEQ ID NO:340. In some aspects, the antibody comprises a Chothia CDR-H2 sequence comprising, consisting of, or consisting essentially of SEQ ID NO:341. In some aspects, the antibody comprises a Chothia CDR-H2 sequence comprising, consisting of, or consisting essentially of SEQ ID NO:342. In some aspects, the antibody comprises a Chothia CDR-H2 sequence comprising, consisting of, or consisting essentially of SEQ ID NO: 343. In some aspects, the antibody comprises a Chothia CDR-H2 sequence comprising, consisting of, or consisting essentially of SEQ ID NO:344. In some aspects, the antibody comprises a Chothia CDR-H2 sequence comprising, consisting of, or consisting essentially of SEQ ID NO:345. In some aspects, the antibody comprises a Chothia CDR-H2 sequence comprising, consisting of, or consisting essentially of SEQ ID NO:346. In some aspects, the antibody comprises a Chothia CDR-H2 sequence comprising, consisting of, or consisting essentially of SEQ ID NO:347. In some aspects, the antibody comprises a Chothia CDR-H2 sequence comprising, consisting of, or consisting essentially of SEQ ID NO: 348. In some aspects, the antibody comprises a Chothia CDR-H2 sequence comprising, consisting of, or consisting essentially of SEQ ID NO:349.

In some aspects, the antibody comprises a Chothia CDR-H2 sequence comprising, consisting of, or consisting essentially of SEQ ID NO:350. In some aspects, the antibody comprises a Chothia CDR-H2 sequence comprising, consisting of, or consisting essentially of SEQ ID NO:351. In some aspects, the antibody comprises a Chothia CDR-H2 sequence comprising, consisting of, or consisting essentially of SEQ ID NO:352. In some aspects, the antibody comprises a Chothia CDR-H2 sequence comprising, consisting of, or consisting essentially of SEQ ID NO:353. In some aspects, the antibody comprises a Chothia CDR-H2 sequence comprising, consisting of, or consisting essentially of SEQ ID NO: 354. In some aspects, the antibody comprises a Chothia CDR-H2 sequence comprising, consisting of, or consisting essentially of SEQ ID NO:355. In some aspects, the antibody comprises a Chothia CDR-H2 sequence comprising, consisting of, or consisting essentially of SEQ ID NO:356. In some aspects, the antibody comprises a Chothia CDR-H2 sequence comprising, consisting of, or consisting essentially of SEQ ID NO:357. In some aspects, the antibody comprises a Chothia CDR-H2 sequence comprising, consisting of, or consisting essentially of SEQ ID NO:358. In some aspects, the antibody comprises a Chothia CDR-H2 sequence comprising, consisting of, or consisting essentially of SEQ ID NO: 359. In some aspects, the antibody comprises a Chothia CDR-H2 sequence comprising, consisting of, or consisting essentially of SEQ ID NO:360. In some aspects, the antibody comprises a Chothia CDR-H2 sequence comprising, consisting of, or consisting essentially of SEQ ID NO:361. In some aspects, the antibody comprises a Chothia CDR-H2 sequence comprising, consisting of, or consisting essentially of SEQ ID NO:362. In some aspects, the antibody comprises a Chothia CDR-H2 sequence comprising, consisting of, or consisting essentially of SEQ ID NO:363. In some aspects, the antibody comprises a Chothia CDR-H2 sequence comprising, consisting of, or consisting essentially of SEQ ID NO: 364. In some aspects, the antibody comprises a Chothia CDR-H2 sequence comprising, consisting of, or consisting essentially of SEQ ID NO:365. In some aspects, the antibody comprises a Chothia CDR-H2 sequence comprising, consisting of, or consisting essentially of SEQ ID NO:366. In some aspects, the antibody comprises a Chothia CDR-H2 sequence comprising, consisting of, or consisting essentially of SEQ ID NO:367. In some aspects, the antibody comprises a Chothia CDR-H2 sequence comprising, consisting of, or consisting essentially of SEQ ID NO:368. In some aspects, the antibody comprises a Chothia CDR-H2 sequence comprising, consisting of, or consisting essentially of SEQ ID NO: 369. In some aspects, the antibody comprises a Chothia CDR-H2 sequence comprising, consisting of, or consisting essentially of SEQ ID NO:370. In some aspects, the antibody comprises a Chothia CDR-H2 sequence comprising, consisting of, or consisting essentially of SEQ ID NO:371. In some aspects, the antibody comprises a Chothia CDR-H2 sequence comprising, consisting of, or consisting essentially of SEQ ID NO:372. In some aspects, the antibody comprises a Chothia CDR-H2 sequence comprising, consisting of, or consisting essentially of SEQ ID NO:373. In some aspects, the antibody comprises a Chothia CDR-H2 sequence comprising, consisting of, or consisting essentially of SEQ ID NO: 374. In some aspects, the antibody comprises a Chothia CDR-H2 sequence comprising, consisting of, or consisting essentially of SEQ ID NO:375. In some aspects, the antibody comprises a Chothia CDR-H2 sequence comprising, consisting of, or consisting essentially of SEQ ID NO:376. In some aspects, the antibody comprises a Chothia CDR-H2 sequence comprising, consisting of, or consisting essentially of SEQ ID NO:377. In some aspects, the antibody comprises a Chothia CDR-H2 sequence comprising, consisting of, or consisting essentially of SEQ ID NO:378. In some aspects, the antibody comprises a Chothia CDR-H2 sequence comprising, consisting of, or consisting essentially of SEQ ID NO: 379. In some aspects, the antibody comprises a Chothia CDR-H2 sequence comprising, consisting of, or consisting essentially of SEQ ID NO:380. In some aspects, the antibody comprises a Chothia CDR-H2 sequence comprising, consisting of, or consisting essentially of SEQ ID NO:381. In some aspects, the antibody comprises a Chothia CDR-H2 sequence comprising, consisting of, or consisting essentially of SEQ ID NO:382. In some aspects, the antibody comprises a Chothia CDR-H2 sequence comprising, consisting of, or consisting essentially of SEQ ID NO:383. In some aspects, the antibody comprises a Chothia CDR-H2 sequence comprising, consisting of, or consisting essentially of SEQ ID NO: 384. In some aspects, the antibody comprises a Chothia CDR-H2 sequence comprising, consisting of, or consisting essentially of SEQ ID NO:385. In some aspects, the antibody comprises a Chothia CDR-H2 sequence comprising, consisting of, or consisting essentially of SEQ ID NO:386. In some aspects, the antibody comprises a Chothia CDR-H2 sequence comprising, consisting of, or consisting essentially of SEQ ID NO:387. In some aspects, the antibody comprises a Chothia CDR-H2 sequence comprising, consisting of, or consisting essentially of SEQ ID NO:388. In some aspects, the antibody comprises a Chothia CDR-H2 sequence comprising, consisting of, or consisting essentially of SEQ ID NO: 389. In some aspects, the antibody comprises a Chothia CDR-H2 sequence comprising, consisting of, or consisting essentially of SEQ ID NO:390. In some aspects, the antibody comprises a Chothia CDR-H2 sequence comprising, consisting of, or consisting essentially of SEQ ID NO:391. In some aspects, the antibody comprises a Chothia CDR-H2 sequence comprising, consisting of, or consisting essentially of SEQ ID NO:392. In some aspects, the antibody comprises a Chothia CDR-H2 sequence comprising, consisting of, or consisting essentially of SEQ ID NO:393. In some aspects, the antibody comprises a Chothia CDR-H2 sequence comprising, consisting of, or consisting essentially of SEQ ID NO: 394. In some aspects, the antibody comprises a Chothia CDR-H2 sequence comprising, consisting of, or consisting essentially of SEQ ID NO:395. In some aspects, the antibody comprises a Chothia CDR-H2 sequence comprising, consisting of, or consisting essentially of SEQ ID NO:396. In some aspects, the antibody comprises a Chothia CDR-H2 sequence comprising, consisting of, or consisting essentially of SEQ ID NO:397. In some aspects, the antibody comprises a Chothia CDR-H2 sequence comprising, consisting of, or consisting essentially of SEQ ID NO:398. In some aspects, the antibody comprises a Chothia CDR-H2 sequence comprising, consisting of, or consisting essentially of SEQ ID NO: 399.

In some aspects, the antibody comprises a Chothia CDR-H2 sequence comprising, consisting of, or consisting essentially of SEQ ID NO:400. In some aspects, the antibody comprises a Chothia CDR-H2 sequence comprising, consisting of, or consisting essentially of SEQ ID NO:401. In some aspects, the antibody comprises a Chothia CDR-H2 sequence comprising, consisting of, or consisting essentially of SEQ ID NO:402. In some aspects, the antibody comprises a Chothia CDR-H2 sequence comprising, consisting of, or consisting essentially of SEQ ID NO:403. In some aspects, the antibody comprises a Chothia CDR-H2 sequence comprising, consisting of, or consisting essentially of SEQ ID NO: 404. In some aspects, the antibody comprises a Chothia CDR-H2 sequence comprising, consisting of, or consisting essentially of SEQ ID NO:405. In some aspects, the antibody comprises a Chothia CDR-H2 sequence comprising, consisting of, or consisting essentially of SEQ ID NO:406. In some aspects, the antibody comprises a Chothia CDR-H2 sequence comprising, consisting of, or consisting essentially of SEQ ID NO:407. In some aspects, the antibody comprises a Chothia CDR-H2 sequence comprising, consisting of, or consisting essentially of SEQ ID NO:408. In some aspects, the antibody comprises a Chothia CDR-H2 sequence comprising, consisting of, or consisting essentially of SEQ ID NO: 409. In some aspects, the antibody comprises a Chothia CDR-H2 sequence comprising, consisting of, or consisting essentially of SEQ ID NO:410. In some aspects, the antibody comprises a Chothia CDR-H2 sequence comprising, consisting of, or consisting essentially of SEQ ID NO:411. In some aspects, the antibody comprises a Chothia CDR-H2 sequence comprising, consisting of, or consisting essentially of SEQ ID NO:412. In some aspects, the antibody comprises a Chothia CDR-H2 sequence comprising, consisting of, or consisting essentially of SEQ ID NO:413. In some aspects, the antibody comprises a Chothia CDR-H2 sequence comprising, consisting of, or consisting essentially of SEQ ID NO: 414. In some aspects, the antibody comprises a Chothia CDR-H2 sequence comprising, consisting of, or consisting essentially of SEQ ID NO:415. In some aspects, the antibody comprises a Chothia CDR-H2 sequence comprising, consisting of, or consisting essentially of SEQ ID NO:416. In some aspects, the antibody comprises a Chothia CDR-H2 sequence comprising, consisting of, or consisting essentially of SEQ ID NO:417. In some aspects, the antibody comprises a Chothia CDR-H2 sequence comprising, consisting of, or consisting essentially of SEQ ID NO:418. In some aspects, the antibody comprises a Chothia CDR-H2 sequence comprising, consisting of, or consisting essentially of SEQ ID NO: 419. In some aspects, the antibody comprises a Chothia CDR-H2 sequence comprising, consisting of, or consisting essentially of SEQ ID NO:420. In some aspects, the antibody comprises a Chothia CDR-H2 sequence comprising, consisting of, or consisting essentially of SEQ ID NO:421. In some aspects, the antibody comprises a Chothia CDR-H2 sequence comprising, consisting of, or consisting essentially of SEQ ID NO:422. In some aspects, the antibody comprises a Chothia CDR-H2 sequence comprising, consisting of, or consisting essentially of SEQ ID NO:423. In some aspects, the antibody comprises a Chothia CDR-H2 sequence comprising, consisting of, or consisting essentially of SEQ ID NO: 424. In some aspects, the antibody comprises a Chothia CDR-H2 sequence comprising, consisting of, or consisting essentially of SEQ ID NO:425. In some aspects, the antibody comprises a Chothia CDR-H2 sequence comprising, consisting of, or consisting essentially of SEQ ID NO:426. In some aspects, the antibody comprises a Chothia CDR-H2 sequence comprising, consisting of, or consisting essentially of SEQ ID NO:427. In some aspects, the antibody comprises a Chothia CDR-H2 sequence comprising, consisting of, or consisting essentially of SEQ ID NO:428. In some aspects, the antibody comprises a Chothia CDR-H2 sequence comprising, consisting of, or consisting essentially of SEQ ID NO: 429. In some aspects, the antibody comprises a Chothia CDR-H2 sequence comprising, consisting of, or consisting essentially of SEQ ID NO:430. In some aspects, the antibody comprises a Chothia CDR-H2 sequence comprising, consisting of, or consisting essentially of SEQ ID NO:431. In some aspects, the antibody comprises a Chothia CDR-H2 sequence comprising, consisting of, or consisting essentially of SEQ ID NO:432. In some aspects, the antibody comprises a Chothia CDR-H2 sequence comprising, consisting of, or consisting essentially of SEQ ID NO:433. In some aspects, the antibody comprises a Chothia CDR-H2 sequence comprising, consisting of, or consisting essentially of SEQ ID NO: 434. In some aspects, the antibody comprises a Chothia CDR-H2 sequence comprising, consisting of, or consisting essentially of SEQ ID NO:435. In some aspects, the antibody comprises a Chothia CDR-H2 sequence comprising, consisting of, or consisting essentially of SEQ ID NO:436. In some aspects, the antibody comprises a Chothia CDR-H2 sequence comprising, consisting of, or consisting essentially of SEQ ID NO:437. In some aspects, the antibody comprises a Chothia CDR-H2 sequence comprising, consisting of, or consisting essentially of SEQ ID NO:438. In some aspects, the antibody comprises a Chothia CDR-H2 sequence comprising, consisting of, or consisting essentially of SEQ ID NO: 439. In some aspects, the antibody comprises a Chothia CDR-H2 sequence comprising, consisting of, or consisting essentially of SEQ ID NO:440. In some aspects, the antibody comprises a Chothia CDR-H2 sequence comprising, consisting of, or consisting essentially of SEQ ID NO:441. In some aspects, the antibody comprises a Chothia CDR-H2 sequence comprising, consisting of, or consisting essentially of SEQ ID NO:442. In some aspects, the antibody comprises a Chothia CDR-H2 sequence comprising, consisting of, or consisting essentially of SEQ ID NO:443. In some aspects, the antibody comprises a Chothia CDR-H2 sequence comprising, consisting of, or consisting essentially of SEQ ID NO: 444. In some aspects, the antibody comprises a Chothia CDR-H2 sequence comprising, consisting of, or consisting essentially of SEQ ID NO:445. In some aspects, the antibody comprises a Chothia CDR-H2 sequence comprising, consisting of, or consisting essentially of SEQ ID NO:446. In some aspects, the antibody comprises a Chothia CDR-H2 sequence comprising, consisting of, or consisting essentially of SEQ ID NO:447. In some aspects, the antibody comprises a Chothia CDR-H2 sequence comprising, consisting of, or consisting essentially of SEQ ID NO:448. In some aspects, the antibody comprises a Chothia CDR-H2 sequence comprising, consisting of, or consisting essentially of SEQ ID NO: 449.

In some aspects, the antibody comprises a Chothia CDR-H2 sequence comprising, consisting of, or consisting essentially of SEQ ID NO:450. In some aspects, the antibody comprises a Chothia CDR-H2 sequence comprising, consisting of, or consisting essentially of SEQ ID NO:451. In some aspects, the antibody comprises a Chothia CDR-H2 sequence comprising, consisting of, or consisting essentially of SEQ ID NO:452. In some aspects, the antibody comprises a Chothia CDR-H2 sequence comprising, consisting of, or consisting essentially of SEQ ID NO:453. In some aspects, the antibody comprises a Chothia CDR-H2 sequence comprising, consisting of, or consisting essentially of SEQ ID NO: 454. In some aspects, the antibody comprises a Chothia CDR-H2 sequence comprising, consisting of, or consisting essentially of SEQ ID NO:455. In some aspects, the antibody comprises a Chothia CDR-H2 sequence comprising, consisting of, or consisting essentially of SEQ ID NO:456. In some aspects, the antibody comprises a Chothia CDR-H2 sequence comprising, consisting of, or consisting essentially of SEQ ID NO:457. In some aspects, the antibody comprises a Chothia CDR-H2 sequence comprising, consisting of, or consisting essentially of SEQ ID NO:458. In some aspects, the antibody comprises a Chothia CDR-H2 sequence comprising, consisting of, or consisting essentially of SEQ ID NO: 459. In some aspects, the antibody comprises a Chothia CDR-H2 sequence comprising, consisting of, or consisting essentially of SEQ ID NO:460. In some aspects, the antibody comprises a Chothia CDR-H2 sequence comprising, consisting of, or consisting essentially of SEQ ID NO:461. In some aspects, the antibody comprises a Chothia CDR-H2 sequence comprising, consisting of, or consisting essentially of SEQ ID NO:462. In some aspects, the antibody comprises a Chothia CDR-H2 sequence comprising, consisting of, or consisting essentially of SEQ ID NO:463. In some aspects, the antibody comprises a Chothia CDR-H2 sequence comprising, consisting of, or consisting essentially of SEQ ID NO: 464. In some aspects, the antibody comprises a Chothia CDR-H2 sequence comprising, consisting of, or consisting essentially of SEQ ID NO:465. In some aspects, the antibody comprises a Chothia CDR-H2 sequence comprising, consisting of, or consisting essentially of SEQ ID NO:466. In some aspects, the antibody comprises a Chothia CDR-H2 sequence comprising, consisting of, or consisting essentially of SEQ ID NO:467. In some aspects, the antibody comprises a Chothia CDR-H2 sequence comprising, consisting of, or consisting essentially of SEQ ID NO:468. In some aspects, the antibody comprises a Chothia CDR-H2 sequence comprising, consisting of, or consisting essentially of SEQ ID NO: 469. In some aspects, the antibody comprises a Chothia CDR-H2 sequence comprising, consisting of, or consisting essentially of SEQ ID NO:470. In some aspects, the antibody comprises a Chothia CDR-H2 sequence comprising, consisting of, or consisting essentially of SEQ ID NO:471. In some aspects, the antibody comprises a Chothia CDR-H2 sequence comprising, consisting of, or consisting essentially of SEQ ID NO:472. In some aspects, the antibody comprises a Chothia CDR-H2 sequence comprising, consisting of, or consisting essentially of SEQ ID NO:473. In some aspects, the antibody comprises a Chothia CDR-H2 sequence comprising, consisting of, or consisting essentially of SEQ ID NO: 474. In some aspects, the antibody comprises a Chothia CDR-H2 sequence comprising, consisting of, or consisting essentially of SEQ ID NO:475. In some aspects, the antibody comprises a Chothia CDR-H2 sequence comprising, consisting of, or consisting essentially of SEQ ID NO:476. In some aspects, the antibody comprises a Chothia CDR-H2 sequence comprising, consisting of, or consisting essentially of SEQ ID NO:477. In some aspects, the antibody comprises a Chothia CDR-H2 sequence comprising, consisting of, or consisting essentially of SEQ ID NO:478. In some aspects, the antibody comprises a Chothia CDR-H2 sequence comprising, consisting of, or consisting essentially of SEQ ID NO: 479. In some aspects, the antibody comprises a Chothia CDR-H2 sequence comprising, consisting of, or consisting essentially of SEQ ID NO:480. In some aspects, the antibody comprises a Chothia CDR-H2 sequence comprising, consisting of, or consisting essentially of SEQ ID NO:481. In some aspects, the antibody comprises a Chothia CDR-H2 sequence comprising, consisting of, or consisting essentially of SEQ ID NO:482. In some aspects, the antibody comprises a Chothia CDR-H2 sequence comprising, consisting of, or consisting essentially of SEQ ID NO:483. In some aspects, the antibody comprises a Chothia CDR-H2 sequence comprising, consisting of, or consisting essentially of SEQ ID NO: 484. In some aspects, the antibody comprises a Chothia CDR-H2 sequence comprising, consisting of, or consisting essentially of SEQ ID NO:485. In some aspects, the antibody comprises a Chothia CDR-H2 sequence comprising, consisting of, or consisting essentially of SEQ ID NO:486. In some aspects, the antibody comprises a Chothia CDR-H2 sequence comprising, consisting of, or consisting essentially of SEQ ID NO:487. In some aspects, the antibody comprises a Chothia CDR-H2 sequence comprising, consisting of, or consisting essentially of SEQ ID NO:488. In some aspects, the antibody comprises a Chothia CDR-H2 sequence comprising, consisting of, or consisting essentially of SEQ ID NO: 489. In some aspects, the antibody comprises a Chothia CDR-H2 sequence comprising, consisting of, or consisting essentially of SEQ ID NO:490. In some aspects, the antibody comprises a Chothia CDR-H2 sequence comprising, consisting of, or consisting essentially of SEQ ID NO:491. In some aspects, the antibody comprises a Chothia CDR-H2 sequence comprising, consisting of, or consisting essentially of SEQ ID NO:492. In some aspects, the antibody comprises a Chothia CDR-H2 sequence comprising, consisting of, or consisting essentially of SEQ ID NO:493. In some aspects, the antibody comprises a Chothia CDR-H2 sequence comprising, consisting of, or consisting essentially of SEQ ID NO: 494. In some aspects, the antibody comprises a Chothia CDR-H2 sequence comprising, consisting of, or consisting essentially of SEQ ID NO:495. In some aspects, the antibody comprises a Chothia CDR-H2 sequence comprising, consisting of, or consisting essentially of SEQ ID NO:496. In some aspects, the antibody comprises a Chothia CDR-H2 sequence comprising, consisting of, or consisting essentially of SEQ ID NO:497. In some aspects, the antibody comprises a Chothia CDR-H2 sequence comprising, consisting of, or consisting essentially of SEQ ID NO:498. In some aspects, the antibody comprises a Chothia CDR-H2 sequence comprising, consisting of, or consisting essentially of SEQ ID NO: 499. In some aspects, the antibody comprises a Chothia CDR-H2 sequence comprising, consisting of, or consisting essentially of SEQ ID NO:500. In some aspects, the antibody comprises a Chothia CDR-H2 sequence comprising, consisting of, or consisting essentially of SEQ ID NO:501. In some aspects, the antibody comprises a Chothia CDR-H2 sequence comprising, consisting of, or consisting essentially of SEQ ID NO:502. In some aspects, the antibody comprises a Chothia CDR-H2 sequence comprising, consisting of, or consisting essentially of SEQ ID NO:503. In some aspects, the antibody comprises a Chothia CDR-H2 sequence comprising, consisting of, or consisting essentially of SEQ ID NO: 504.

### 5.2.2.3. Chothia CDR-H1

In some embodiments, the antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of a V_{H} sequence provided in SEQ ID NOs: 854-1020.

In some embodiments, the antibody comprises a V_{H} sequence comprising a Chothia CDR-H1 sequence comprising, consisting of, or consisting essentially of a sequence selected from SEQ ID NOs: 4-170. In some aspects, the antibody comprises a Chothia CDR-H1 sequence comprising, consisting of, or consisting essentially of SEQ ID NO: 4. In some aspects, the antibody comprises a Chothia CDR-H1 sequence comprising, consisting of, or consisting essentially of SEQ ID NO:5. In some aspects, the antibody comprises a Chothia CDR-H1 sequence comprising, consisting of, or consisting essentially of SEQ ID NO:6. In some aspects, the antibody comprises a Chothia CDR-H1 sequence comprising, consisting of, or consisting essentially of SEQ ID NO:7. In some aspects, the antibody comprises a Chothia CDR-H1 sequence comprising, consisting of, or consisting essentially of SEQ ID NO:8. In some aspects, the antibody comprises a Chothia CDR-H1 sequence comprising, consisting of, or consisting essentially of SEQ ID NO:9. In some aspects, the antibody comprises a Chothia CDR-H1 sequence comprising, consisting of, or consisting essentially of SEQ ID NO:10. In some aspects, the antibody comprises a Chothia CDR-H1 sequence comprising, consisting of, or consisting essentially of SEQ ID NO: 11. In some aspects, the antibody comprises a Chothia CDR-H1 sequence comprising, consisting of, or consisting essentially of SEQ ID NO:12. In some aspects, the antibody comprises a Chothia CDR-H1 sequence comprising, consisting of, or consisting essentially of SEQ ID NO:13. In some aspects, the antibody comprises a Chothia CDR-H1 sequence comprising, consisting of, or consisting essentially of SEQ ID NO:14. In some aspects, the antibody comprises a Chothia CDR-H1 sequence comprising, consisting of, or consisting essentially of SEQ ID NO:15. In some aspects, the antibody comprises a Chothia CDR-H1 sequence comprising, consisting of, or consisting essentially of SEQ ID NO:16. In some aspects, the antibody comprises a Chothia CDR-H1 sequence comprising, consisting of, or consisting essentially of SEQ ID NO:17. In some aspects, the antibody comprises a Chothia CDR-H1 sequence comprising, consisting of, or consisting essentially of SEQ ID NO: 18. In some aspects, the antibody comprises a Chothia CDR-H1 sequence comprising, consisting of, or consisting essentially of SEQ ID NO:19. In some aspects, the antibody comprises a Chothia CDR-H1 sequence comprising, consisting of, or consisting essentially of SEQ ID NO:20. In some aspects, the antibody comprises a Chothia CDR-H1 sequence comprising, consisting of, or consisting essentially of SEQ ID NO:21. In some aspects, the antibody comprises a Chothia CDR-H1 sequence comprising, consisting of, or consisting essentially of SEQ ID NO:22. In some aspects, the antibody comprises a Chothia CDR-H1 sequence comprising, consisting of, or consisting essentially of SEQ ID NO:23. In some aspects, the antibody comprises a Chothia CDR-H1 sequence comprising, consisting of, or consisting essentially of SEQ ID NO:24. In some aspects, the antibody comprises a Chothia CDR-H1 sequence comprising, consisting of, or consisting essentially of SEQ ID NO: 25. In some aspects, the antibody comprises a Chothia CDR-H1 sequence comprising, consisting of, or consisting essentially of SEQ ID NO:26. In some aspects, the antibody comprises a Chothia CDR-H1 sequence comprising, consisting of, or consisting essentially of SEQ ID NO:27. In some aspects, the antibody comprises a Chothia CDR-H1 sequence comprising, consisting of, or consisting essentially of SEQ ID NO:28. In some aspects, the antibody comprises a Chothia CDR-H1 sequence comprising, consisting of, or consisting essentially of SEQ ID NO:29. In some aspects, the antibody comprises a Chothia CDR-H1 sequence comprising, consisting of, or consisting essentially of SEQ ID NO:30. In some aspects, the antibody comprises a Chothia CDR-H1 sequence comprising, consisting of, or consisting essentially of SEQ ID NO:31. In some aspects, the antibody comprises a Chothia CDR-H1 sequence comprising, consisting of, or consisting essentially of SEQ ID NO: 32. In some aspects, the antibody comprises a Chothia CDR-H1 sequence comprising, consisting of, or consisting essentially of SEQ ID NO:33. In some aspects, the antibody comprises a Chothia CDR-H1 sequence comprising, consisting of, or consisting essentially of SEQ ID NO:34. In some aspects, the antibody comprises a Chothia CDR-H1 sequence comprising, consisting of, or consisting essentially of SEQ ID NO:35. In some aspects, the antibody comprises a Chothia CDR-H1 sequence comprising, consisting of, or consisting essentially of SEQ ID NO:36. In some aspects, the antibody comprises a Chothia CDR-H1 sequence comprising, consisting of, or consisting essentially of SEQ ID NO:37. In some aspects, the antibody comprises a Chothia CDR-H1 sequence comprising, consisting of, or consisting essentially of SEQ ID NO:38. In some aspects, the antibody comprises a Chothia CDR-H1 sequence comprising, consisting of, or consisting essentially of SEQ ID NO: 39. In some aspects, the antibody comprises a Chothia CDR-H1 sequence comprising, consisting of, or consisting essentially of SEQ ID NO:40. In some aspects, the antibody comprises a Chothia CDR-H1 sequence comprising, consisting of, or consisting essentially of SEQ ID NO:41. In some aspects, the antibody comprises a Chothia CDR-H1 sequence comprising, consisting of, or consisting essentially of SEQ ID NO:42. In some aspects, the antibody comprises a Chothia CDR-H1 sequence comprising, consisting of, or consisting essentially of SEQ ID NO:43. In some aspects, the antibody comprises a Chothia CDR-H1 sequence comprising, consisting of, or consisting essentially of SEQ ID NO:44. In some aspects, the antibody comprises a Chothia CDR-H1 sequence comprising, consisting of, or consisting essentially of SEQ ID NO:45. In some aspects, the antibody comprises a Chothia CDR-H1 sequence comprising, consisting of, or consisting essentially of SEQ ID NO: 46. In some aspects, the antibody comprises a Chothia CDR-H1 sequence comprising, consisting of, or consisting essentially of SEQ ID NO:47. In some aspects, the antibody comprises a Chothia CDR-H1 sequence comprising, consisting of, or consisting essentially of SEQ ID NO:48. In some aspects, the antibody comprises a Chothia CDR-H1 sequence comprising, consisting of, or consisting essentially of SEQ ID NO:49.

In some aspects, the antibody comprises a Chothia CDR-H1 sequence comprising, consisting of, or consisting essentially of SEQ ID NO:50. In some aspects, the antibody comprises a Chothia CDR-H1 sequence comprising, consisting of, or consisting essentially of SEQ ID NO:51. In some aspects, the antibody comprises a Chothia CDR-H1 sequence comprising, consisting of, or consisting essentially of SEQ ID NO:52. In some aspects, the antibody comprises a Chothia CDR-H1 sequence comprising, consisting of, or consisting essentially of SEQ ID NO:53. In some aspects, the antibody comprises a Chothia CDR-H1 sequence comprising, consisting of, or consisting essentially of SEQ ID NO:54. In some aspects, the antibody comprises a Chothia CDR-H1 sequence comprising, consisting of, or consisting essentially of SEQ ID NO:55. In some aspects, the antibody comprises a Chothia CDR-H1 sequence comprising, consisting of, or consisting essentially of SEQ ID NO: 56. In some aspects, the antibody comprises a Chothia CDR-H1 sequence comprising, consisting of, or consisting essentially of SEQ ID NO:57. In some aspects, the antibody comprises a Chothia CDR-H1 sequence comprising, consisting of, or consisting essentially of SEQ ID NO:58. In some aspects, the antibody comprises a Chothia CDR-H1 sequence comprising, consisting of, or consisting essentially of SEQ ID NO:59. In some aspects, the antibody comprises a Chothia CDR-H1 sequence comprising, consisting of, or consisting essentially of SEQ ID NO:60. In some aspects, the antibody comprises a Chothia CDR-H1 sequence comprising, consisting of, or consisting essentially of SEQ ID NO:61. In some aspects, the antibody comprises a Chothia CDR-H1 sequence comprising, consisting of, or consisting essentially of SEQ ID NO:62. In some aspects, the antibody comprises a Chothia CDR-H1 sequence comprising, consisting of, or consisting essentially of SEQ ID NO: 63. In some aspects, the antibody comprises a Chothia CDR-H1 sequence comprising, consisting of, or consisting essentially of SEQ ID NO:64. In some aspects, the antibody comprises a Chothia CDR-H1 sequence comprising, consisting of, or consisting essentially of SEQ ID NO:65. In some aspects, the antibody comprises a Chothia CDR-H1 sequence comprising, consisting of, or consisting essentially of SEQ ID NO:66. In some aspects, the antibody comprises a Chothia CDR-H1 sequence comprising, consisting of, or consisting essentially of SEQ ID NO:67. In some aspects, the antibody comprises a Chothia CDR-H1 sequence comprising, consisting of, or consisting essentially of SEQ ID NO:68. In some aspects, the antibody comprises a Chothia CDR-H1 sequence comprising, consisting of, or consisting essentially of SEQ ID NO:69. In some aspects, the antibody comprises a Chothia CDR-H1 sequence comprising, consisting of, or consisting essentially of SEQ ID NO: 70. In some aspects, the antibody comprises a Chothia CDR-H1 sequence comprising, consisting of, or consisting essentially of SEQ ID NO:71. In some aspects, the antibody comprises a Chothia CDR-H1 sequence comprising, consisting of, or consisting essentially of SEQ ID NO:72. In some aspects, the antibody comprises a Chothia CDR-H1 sequence comprising, consisting of, or consisting essentially of SEQ ID NO:73. In some aspects, the antibody comprises a Chothia CDR-H1 sequence comprising, consisting of, or consisting essentially of SEQ ID NO:74. In some aspects, the antibody comprises a Chothia CDR-H1 sequence comprising, consisting of, or consisting essentially of SEQ ID NO:75. In some aspects, the antibody comprises a Chothia CDR-H1 sequence comprising, consisting of, or consisting essentially of SEQ ID NO:76. In some aspects, the antibody comprises a Chothia CDR-H1 sequence comprising, consisting of, or consisting essentially of SEQ ID NO: 77. In some aspects, the antibody comprises a Chothia CDR-H1 sequence comprising, consisting of, or consisting essentially of SEQ ID NO:78. In some aspects, the antibody comprises a Chothia CDR-H1 sequence comprising, consisting of, or consisting essentially of SEQ ID NO:79. In some aspects, the antibody comprises a Chothia CDR-H1 sequence comprising, consisting of, or consisting essentially of SEQ ID NO:80. In some aspects, the antibody comprises a Chothia CDR-H1 sequence comprising, consisting of, or consisting essentially of SEQ ID NO:81. In some aspects, the antibody comprises a Chothia CDR-H1 sequence comprising, consisting of, or consisting essentially of SEQ ID NO:82. In some aspects, the antibody comprises a Chothia CDR-H1 sequence comprising, consisting of, or consisting essentially of SEQ ID NO:83. In some aspects, the antibody comprises a Chothia CDR-H1 sequence comprising, consisting of, or consisting essentially of SEQ ID NO: 84. In some aspects, the antibody comprises a Chothia CDR-H1 sequence comprising, consisting of, or consisting essentially of SEQ ID NO:85. In some aspects, the antibody comprises a Chothia CDR-H1 sequence comprising, consisting of, or consisting essentially of SEQ ID NO:86. In some aspects, the antibody comprises a Chothia CDR-H1 sequence comprising, consisting of, or consisting essentially of SEQ ID NO:87. In some aspects, the antibody comprises a Chothia CDR-H1 sequence comprising, consisting of, or consisting essentially of SEQ ID NO:88. In some aspects, the antibody comprises a Chothia CDR-H1 sequence comprising, consisting of, or consisting essentially of SEQ ID NO:89. In some aspects, the antibody comprises a Chothia CDR-H1 sequence comprising, consisting of, or consisting essentially of SEQ ID NO:90. In some aspects, the antibody comprises a Chothia CDR-H1 sequence comprising, consisting of, or consisting essentially of SEQ ID NO: 91. In some aspects, the antibody comprises a Chothia CDR-H1 sequence comprising, consisting of, or consisting essentially of SEQ ID NO:92. In some aspects, the antibody comprises a Chothia CDR-H1 sequence comprising, consisting of, or consisting essentially of SEQ ID NO:93. In some aspects, the antibody comprises a Chothia CDR-H1 sequence comprising, consisting of, or consisting essentially of SEQ ID NO:94. In some aspects, the antibody comprises a Chothia CDR-H1 sequence comprising, consisting of, or consisting essentially of SEQ ID NO:95. In some aspects, the antibody comprises a Chothia CDR-H1 sequence comprising, consisting of, or consisting essentially of SEQ ID NO:96. In some aspects, the antibody comprises a Chothia CDR-H1 sequence comprising, consisting of, or consisting essentially of SEQ ID NO:97. In some aspects, the antibody comprises a Chothia CDR-H1 sequence comprising, consisting of, or consisting essentially of SEQ ID NO: 98. In some aspects, the antibody comprises a Chothia CDR-H1 sequence comprising, consisting of, or consisting essentially of SEQ ID NO:99.

In some aspects, the antibody comprises a Chothia CDR-H1 sequence comprising, consisting of, or consisting essentially of SEQ ID NO:100. In some aspects, the antibody comprises a Chothia CDR-H1 sequence comprising, consisting of, or consisting essentially of SEQ ID NO:101. In some aspects, the antibody comprises a Chothia CDR-H1 sequence comprising, consisting of, or consisting essentially of SEQ ID NO:102. In some aspects, the antibody comprises a Chothia CDR-H1 sequence comprising, consisting of, or consisting essentially of SEQ ID NO:103. In some aspects, the antibody comprises a Chothia CDR-H1 sequence comprising, consisting of, or consisting essentially of SEQ ID NO: 104. In some aspects, the antibody comprises a Chothia CDR-H1 sequence comprising, consisting of, or consisting essentially of SEQ ID NO:105. In some aspects, the antibody comprises a Chothia CDR-H1 sequence comprising, consisting of, or consisting essentially of SEQ ID NO:106. In some aspects, the antibody comprises a Chothia CDR-H1 sequence comprising, consisting of, or consisting essentially of SEQ ID NO:107. In some aspects, the antibody comprises a Chothia CDR-H1 sequence comprising, consisting of, or consisting essentially of SEQ ID NO:108. In some aspects, the antibody comprises a Chothia CDR-H1 sequence comprising, consisting of, or consisting essentially of SEQ ID NO: 109. In some aspects, the antibody comprises a Chothia CDR-H1 sequence comprising, consisting of, or consisting essentially of SEQ ID NO:110. In some aspects, the antibody comprises a Chothia CDR-H1 sequence comprising, consisting of, or consisting essentially of SEQ ID NO:111. In some aspects, the antibody comprises a Chothia CDR-H1 sequence comprising, consisting of, or consisting essentially of SEQ ID NO:112. In some aspects, the antibody comprises a Chothia CDR-H1 sequence comprising, consisting of, or consisting essentially of SEQ ID NO:113. In some aspects, the antibody comprises a Chothia CDR-H1 sequence comprising, consisting of, or consisting essentially of SEQ ID NO: 114. In some aspects, the antibody comprises a Chothia CDR-H1 sequence comprising, consisting of, or consisting essentially of SEQ ID NO:115. In some aspects, the antibody comprises a Chothia CDR-H1 sequence comprising, consisting of, or consisting essentially of SEQ ID NO:116. In some aspects, the antibody comprises a Chothia CDR-H1 sequence comprising, consisting of, or consisting essentially of SEQ ID NO:117. In some aspects, the antibody comprises a Chothia CDR-H1 sequence comprising, consisting of, or consisting essentially of SEQ ID NO:118. In some aspects, the antibody comprises a Chothia CDR-H1 sequence comprising, consisting of, or consisting essentially of SEQ ID NO: 119. In some aspects, the antibody comprises a Chothia CDR-H1 sequence comprising, consisting of, or consisting essentially of SEQ ID NO:120. In some aspects, the antibody comprises a Chothia CDR-H1 sequence comprising, consisting of, or consisting essentially of SEQ ID NO:121. In some aspects, the antibody comprises a Chothia CDR-H1 sequence comprising, consisting of, or consisting essentially of SEQ ID NO:122. In some aspects, the antibody comprises a Chothia CDR-H1 sequence comprising, consisting of, or consisting essentially of SEQ ID NO:123. In some aspects, the antibody comprises a Chothia CDR-H1 sequence comprising, consisting of, or consisting essentially of SEQ ID NO: 124. In some aspects, the antibody comprises a Chothia CDR-H1 sequence comprising, consisting of, or consisting essentially of SEQ ID NO:125. In some aspects, the antibody comprises a Chothia CDR-H1 sequence comprising, consisting of, or consisting essentially of SEQ ID NO:126. In some aspects, the antibody comprises a Chothia CDR-H1 sequence comprising, consisting of, or consisting essentially of SEQ ID NO:127. In some aspects, the antibody comprises a Chothia CDR-H1 sequence comprising, consisting of, or consisting essentially of SEQ ID NO:128. In some aspects, the antibody comprises a Chothia CDR-H1 sequence comprising, consisting of, or consisting essentially of SEQ ID NO: 129. In some aspects, the antibody comprises a Chothia CDR-H1 sequence comprising, consisting of, or consisting essentially of SEQ ID NO:130. In some aspects, the antibody comprises a Chothia CDR-H1 sequence comprising, consisting of, or consisting essentially of SEQ ID NO:131. In some aspects, the antibody comprises a Chothia CDR-H1 sequence comprising, consisting of, or consisting essentially of SEQ ID NO:132. In some aspects, the antibody comprises a Chothia CDR-H1 sequence comprising, consisting of, or consisting essentially of SEQ ID NO:133. In some aspects, the antibody comprises a Chothia CDR-H1 sequence comprising, consisting of, or consisting essentially of SEQ ID NO: 134. In some aspects, the antibody comprises a Chothia CDR-H1 sequence comprising, consisting of, or consisting essentially of SEQ ID NO:135. In some aspects, the antibody comprises a Chothia CDR-H1 sequence comprising, consisting of, or consisting essentially of SEQ ID NO:136. In some aspects, the antibody comprises a Chothia CDR-H1 sequence comprising, consisting of, or consisting essentially of SEQ ID NO:137. In some aspects, the antibody comprises a Chothia CDR-H1 sequence comprising, consisting of, or consisting essentially of SEQ ID NO:138. In some aspects, the antibody comprises a Chothia CDR-H1 sequence comprising, consisting of, or consisting essentially of SEQ ID NO: 139. In some aspects, the antibody comprises a Chothia CDR-H1 sequence comprising, consisting of, or consisting essentially of SEQ ID NO:140. In some aspects, the antibody comprises a Chothia CDR-H1 sequence comprising, consisting of, or consisting essentially of SEQ ID NO:141. In some aspects, the antibody comprises a Chothia CDR-H1 sequence comprising, consisting of, or consisting essentially of SEQ ID NO:142. In some aspects, the antibody comprises a Chothia CDR-H1 sequence comprising, consisting of, or consisting essentially of SEQ ID NO:143. In some aspects, the antibody comprises a Chothia CDR-H1 sequence comprising, consisting of, or consisting essentially of SEQ ID NO: 144. In some aspects, the antibody comprises a Chothia CDR-H1 sequence comprising, consisting of, or consisting essentially of SEQ ID NO:145. In some aspects, the antibody comprises a Chothia CDR-H1 sequence comprising, consisting of, or consisting essentially of SEQ ID NO:146. In some aspects, the antibody comprises a Chothia CDR-H1 sequence comprising, consisting of, or consisting essentially of SEQ ID NO:147. In some aspects, the antibody comprises a Chothia CDR-H1 sequence comprising, consisting of, or consisting essentially of SEQ ID NO:148. In some aspects, the antibody comprises a Chothia CDR-H1 sequence comprising, consisting of, or consisting essentially of SEQ ID NO: 149.

In some aspects, the antibody comprises a Chothia CDR-H1 sequence comprising, consisting of, or consisting essentially of SEQ ID NO:150. In some aspects, the antibody comprises a Chothia CDR-H1 sequence comprising, consisting of, or consisting essentially of SEQ ID NO:151. In some aspects, the antibody comprises a Chothia CDR-H1 sequence comprising, consisting of, or consisting essentially of SEQ ID NO:152. In some aspects, the antibody comprises a Chothia CDR-H1 sequence comprising, consisting of, or consisting essentially of SEQ ID NO:153. In some aspects, the antibody comprises a Chothia CDR-H1 sequence comprising, consisting of, or consisting essentially of SEQ ID NO: 154. In some aspects, the antibody comprises a Chothia CDR-H1 sequence comprising, consisting of, or consisting essentially of SEQ ID NO:155. In some aspects, the antibody comprises a Chothia CDR-H1 sequence comprising, consisting of, or consisting essentially of SEQ ID NO:156. In some aspects, the antibody comprises a Chothia CDR-H1 sequence comprising, consisting of, or consisting essentially of SEQ ID NO:157. In some aspects, the antibody comprises a Chothia CDR-H1 sequence comprising, consisting of, or consisting essentially of SEQ ID NO:158. In some aspects, the antibody comprises a Chothia CDR-H1 sequence comprising, consisting of, or consisting essentially of SEQ ID NO: 159. In some aspects, the antibody comprises a Chothia CDR-H1 sequence comprising, consisting of, or consisting essentially of SEQ ID NO:160. In some aspects, the antibody comprises a Chothia CDR-H1 sequence comprising, consisting of, or consisting essentially of SEQ ID NO:161. In some aspects, the antibody comprises a Chothia CDR-H1 sequence comprising, consisting of, or consisting essentially of SEQ ID NO:162. In some aspects, the antibody comprises a Chothia CDR-H1 sequence comprising, consisting of, or consisting essentially of SEQ ID NO:163. In some aspects, the antibody comprises a Chothia CDR-H1 sequence comprising, consisting of, or consisting essentially of SEQ ID NO: 164. In some aspects, the antibody comprises a Chothia CDR-H1 sequence comprising, consisting of, or consisting essentially of SEQ ID NO:165. In some aspects, the antibody comprises a Chothia CDR-H1 sequence comprising, consisting of, or consisting essentially of SEQ ID NO:166. In some aspects, the antibody comprises a Chothia CDR-H1 sequence comprising, consisting of, or consisting essentially of SEQ ID NO:167. In some aspects, the antibody comprises a Chothia CDR-H1 sequence comprising, consisting of, or consisting essentially of SEQ ID NO:168. In some aspects, the antibody comprises a Chothia CDR-H1 sequence comprising, consisting of, or consisting essentially of SEQ ID NO: 169. In some aspects, the antibody comprises a Chothia CDR-H1 sequence comprising, consisting of, or consisting essentially of SEQ ID NO:170.

### 5.2.2.4. Chothia CDR-H3+Chothia CDR-H2

In some embodiments, the antibody comprises a V_{H} sequence comprising a Chothia CDR-H3 sequence comprising, consisting of, or consisting essentially of a sequence selected from SEQ ID NOs: 672-838, and a Chothia CDR-H2 sequence comprising, consisting of, or consisting essentially of a sequence selected from SEQ ID NOs: 338-504. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 854-1020.

### 5.2.2.5. Chothia CDR-H3+Chothia CDR-H1

In some embodiments, the antibody comprises a V_{H} sequence comprising a Chothia CDR-H3 sequence comprising, consisting of, or consisting essentially of a sequence selected from SEQ ID NOs: 672-838, and a Chothia CDR-H1 sequence comprising, consisting of, or consisting essentially of a sequence selected from SEQ ID NOs: 4-170. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 854-1020.

### 5.2.2.6. Chothia CDR-H1+Chothia CDR-H2

In some embodiments, the antibody comprises a V_{H} sequence comprising a Chothia CDR-H1 sequence comprising, consisting of, or consisting essentially of a sequence selected from SEQ ID NOs: 4-170 and a Chothia CDR-H2 sequence comprising, consisting of, or consisting essentially of a sequence selected from SEQ ID NOs: 338-504. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 854-1020.

### 5.2.2.7. Chothia CDR-H1+Chothia CDR-H2+Chothia CDR-H3

In some embodiments, the antibody comprises a V_{H} sequence comprising a Chothia CDR-H1 sequence comprising, consisting of, or consisting essentially of a sequence selected from SEQ ID NOs: 4-170, a Chothia CDR-H2 sequence comprising, consisting of, or consisting essentially of a sequence selected from SEQ ID NOs: 1338-504, and a Chothia CDR-H3 sequence comprising, consisting of, or consisting essentially of a sequence selected from SEQ ID NOs: 672-838. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 854-1020.

### 5.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, 95%, 96%, 97%, 98%, or 99% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, 95%, 96%, 97%, 98%, or 99% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, 95%, 96%, 97%, 98%, or 99% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

### 5.3. V_{H} Sequences

In some embodiments, the antibody comprises, consists of, or consists essentially of a V_{H} sequence provided in SEQ ID NOs: 854-1020.

In some embodiments, the antibody comprises a V_{H} sequence comprising, consisting of, or consisting essentially of a sequence selected from SEQ ID NOs: 854-1020. In some aspects, the antibody comprises a V_{H} sequence comprising, consisting of, or consisting essentially of SEQ ID NO:854. In some aspects, the antibody comprises a V_{H} sequence comprising, consisting of, or consisting essentially of SEQ ID NO:855. In some aspects, the antibody comprises a V_{H} sequence comprising, consisting of, or consisting essentially of SEQ ID NO:856. In some aspects, the antibody comprises a V_{H} sequence comprising, consisting of, or consisting essentially of SEQ ID NO:857. In some aspects, the antibody comprises a V_{H} sequence comprising, consisting of, or consisting essentially of SEQ ID NO:858. In some aspects, the antibody comprises a V_{H} sequence comprising, consisting of, or consisting essentially of SEQ ID NO:859. In some aspects, the antibody comprises a V_{H} sequence comprising, consisting of, or consisting essentially of SEQ ID NO: 860. In some aspects, the antibody comprises a V_{H} sequence comprising, consisting of, or consisting essentially of SEQ ID NO:861. In some aspects, the antibody comprises a V_{H} sequence comprising, consisting of, or consisting essentially of SEQ ID NO:862. In some aspects, the antibody comprises a V_{H} sequence comprising, consisting of, or consisting essentially of SEQ ID NO:863. In some aspects, the antibody comprises a V_{H} sequence comprising, consisting of, or consisting essentially of SEQ ID NO:864. In some aspects, the antibody comprises a V_{H} sequence comprising, consisting of, or consisting essentially of SEQ ID NO:865. In some aspects, the antibody comprises a V_{H} sequence comprising, consisting of, or consisting essentially of SEQ ID NO:866. In some aspects, the antibody comprises a V_{H} sequence comprising, consisting of, or consisting essentially of SEQ ID NO: 867. In some aspects, the antibody comprises a V_{H} sequence comprising, consisting of, or consisting essentially of SEQ ID NO:868. In some aspects, the antibody comprises a V_{H} sequence comprising, consisting of, or consisting essentially of SEQ ID NO:869. In some aspects, the antibody comprises a V_{H} sequence comprising, consisting of, or consisting essentially of SEQ ID NO:870. In some aspects, the antibody comprises a V_{H} sequence comprising, consisting of, or consisting essentially of SEQ ID NO:871. In some aspects, the antibody comprises a V_{H} sequence comprising, consisting of, or consisting essentially of SEQ ID NO:872. In some aspects, the antibody comprises a V_{H} sequence comprising, consisting of, or consisting essentially of SEQ ID NO:873. In some aspects, the antibody comprises a V_{H} sequence comprising, consisting of, or consisting essentially of SEQ ID NO: 874. In some aspects, the antibody comprises a V_{H} sequence comprising, consisting of, or consisting essentially of SEQ ID NO:875. In some aspects, the antibody comprises a V_{H} sequence comprising, consisting of, or consisting essentially of SEQ ID NO:876. In some aspects, the antibody comprises a V_{H} sequence comprising, consisting of, or consisting essentially of SEQ ID NO:877. In some aspects, the antibody comprises a V_{H} sequence comprising, consisting of, or consisting essentially of SEQ ID NO:878. In some aspects, the antibody comprises a V_{H} sequence comprising, consisting of, or consisting essentially of SEQ ID NO:879. In some aspects, the antibody comprises a V_{H} sequence comprising, consisting of, or consisting essentially of SEQ ID NO:880. In some aspects, the antibody comprises a V_{H} sequence comprising, consisting of, or consisting essentially of SEQ ID NO: 881. In some aspects, the antibody comprises a V_{H} sequence comprising, consisting of, or consisting essentially of SEQ ID NO:882. In some aspects, the antibody comprises a V_{H} sequence comprising, consisting of, or consisting essentially of SEQ ID NO:883. In some aspects, the antibody comprises a V_{H} sequence comprising, consisting of, or consisting essentially of SEQ ID NO:884. In some aspects, the antibody comprises a V_{H} sequence comprising, consisting of, or consisting essentially of SEQ ID NO:885. In some aspects, the antibody comprises a V_{H} sequence comprising, consisting of, or consisting essentially of SEQ ID NO:886. In some aspects, the antibody comprises a V_{H} sequence comprising, consisting of, or consisting essentially of SEQ ID NO:887. In some aspects, the antibody comprises a V_{H} sequence comprising, consisting of, or consisting essentially of SEQ ID NO: 888. In some aspects, the antibody comprises a V_{H} sequence comprising, consisting of, or consisting essentially of SEQ ID NO:889. In some aspects, the antibody comprises a V_{H} sequence comprising, consisting of, or consisting essentially of SEQ ID NO:890. In some aspects, the antibody comprises a V_{H} sequence comprising, consisting of, or consisting essentially of SEQ ID NO:891. In some aspects, the antibody comprises a V_{H} sequence comprising, consisting of, or consisting essentially of SEQ ID NO:892. In some aspects, the antibody comprises a V_{H} sequence comprising, consisting of, or consisting essentially of SEQ ID NO:893. In some aspects, the antibody comprises a V_{H} sequence comprising, consisting of, or consisting essentially of SEQ ID NO:894. In some aspects, the antibody comprises a V_{H} sequence comprising, consisting of, or consisting essentially of SEQ ID NO: 895. In some aspects, the antibody comprises a V_{H} sequence comprising, consisting of, or consisting essentially of SEQ ID NO:896. In some aspects, the antibody comprises a V_{H} sequence comprising, consisting of, or consisting essentially of SEQ ID NO:897. In some aspects, the antibody comprises a V_{H} sequence comprising, consisting of, or consisting essentially of SEQ ID NO:898. In some aspects, the antibody comprises a V_{H} sequence comprising, consisting of, or consisting essentially of SEQ ID NO:899.

In some aspects, the antibody comprises a V_{H} sequence comprising, consisting of, or consisting essentially of SEQ ID NO:900. In some aspects, the antibody comprises a V_{H} sequence comprising, consisting of, or consisting essentially of SEQ ID NO:901. In some aspects, the antibody comprises a V_{H} sequence comprising, consisting of, or consisting essentially of SEQ ID NO:902. In some aspects, the antibody comprises a V_{H} sequence comprising, consisting of, or consisting essentially of SEQ ID NO:903. In some aspects, the antibody comprises a V_{H} sequence comprising, consisting of, or consisting essentially of SEQ ID NO:904. In some aspects, the antibody comprises a V_{H} sequence comprising, consisting of, or consisting essentially of SEQ ID NO:905. In some aspects, the antibody comprises a V_{H} sequence comprising, consisting of, or consisting essentially of SEQ ID NO: 906. In some aspects, the antibody comprises a V_{H} sequence comprising, consisting of, or consisting essentially of SEQ ID NO:907. In some aspects, the antibody comprises a V_{H} sequence comprising, consisting of, or consisting essentially of SEQ ID NO:908. In some aspects, the antibody comprises a V_{H} sequence comprising, consisting of, or consisting essentially of SEQ ID NO:909. In some aspects, the antibody comprises a V_{H} sequence comprising, consisting of, or consisting essentially of SEQ ID NO:910. In some aspects, the antibody comprises a V_{H} sequence comprising, consisting of, or consisting essentially of SEQ ID NO:911. In some aspects, the antibody comprises a V_{H} sequence comprising, consisting of, or consisting essentially of SEQ ID NO:912. In some aspects, the antibody comprises a V_{H} sequence comprising, consisting of, or consisting essentially of SEQ ID NO: 913. In some aspects, the antibody comprises a V_{H} sequence comprising, consisting of, or consisting essentially of SEQ ID NO:914. In some aspects, the antibody comprises a V_{H} sequence comprising, consisting of, or consisting essentially of SEQ ID NO:915. In some aspects, the antibody comprises a V_{H} sequence comprising, consisting of, or consisting essentially of SEQ ID NO:916. In some aspects, the antibody comprises a V_{H} sequence comprising, consisting of, or consisting essentially of SEQ ID NO:917. In some aspects, the antibody comprises a V_{H} sequence comprising, consisting of, or consisting essentially of SEQ ID NO:918. In some aspects, the antibody comprises a V_{H} sequence comprising, consisting of, or consisting essentially of SEQ ID NO:919. In some aspects, the antibody comprises a V_{H} sequence comprising, consisting of, or consisting essentially of SEQ ID NO: 920. In some aspects, the antibody comprises a V_{H} sequence comprising, consisting of, or consisting essentially of SEQ ID NO:921. In some aspects, the antibody comprises a V_{H} sequence comprising, consisting of, or consisting essentially of SEQ ID NO:922. In some aspects, the antibody comprises a V_{H} sequence comprising, consisting of, or consisting essentially of SEQ ID NO:923. In some aspects, the antibody comprises a V_{H} sequence comprising, consisting of, or consisting essentially of SEQ ID NO:924. In some aspects, the antibody comprises a V_{H} sequence comprising, consisting of, or consisting essentially of SEQ ID NO:925. In some aspects, the antibody comprises a V_{H} sequence comprising, consisting of, or consisting essentially of SEQ ID NO:926. In some aspects, the antibody comprises a V_{H} sequence comprising, consisting of, or consisting essentially of SEQ ID NO: 927. In some aspects, the antibody comprises a V_{H} sequence comprising, consisting of, or consisting essentially of SEQ ID NO:928. In some aspects, the antibody comprises a V_{H} sequence comprising, consisting of, or consisting essentially of SEQ ID NO:929. In some aspects, the antibody comprises a V_{H} sequence comprising, consisting of, or consisting essentially of SEQ ID NO:930. In some aspects, the antibody comprises a V_{H} sequence comprising, consisting of, or consisting essentially of SEQ ID NO:931. In some aspects, the antibody comprises a V_{H} sequence comprising, consisting of, or consisting essentially of SEQ ID NO:932. In some aspects, the antibody comprises a V_{H} sequence comprising, consisting of, or consisting essentially of SEQ ID NO:933. In some aspects, the antibody comprises a V_{H} sequence comprising, consisting of, or consisting essentially of SEQ ID NO: 934. In some aspects, the antibody comprises a V_{H} sequence comprising, consisting of, or consisting essentially of SEQ ID NO:935. In some aspects, the antibody comprises a V_{H} sequence comprising, consisting of, or consisting essentially of SEQ ID NO:936. In some aspects, the antibody comprises a V_{H} sequence comprising, consisting of, or consisting essentially of SEQ ID NO:937. In some aspects, the antibody comprises a V_{H} sequence comprising, consisting of, or consisting essentially of SEQ ID NO:938. In some aspects, the antibody comprises a V_{H} sequence comprising, consisting of, or consisting essentially of SEQ ID NO:939. In some aspects, the antibody comprises a V_{H} sequence comprising, consisting of, or consisting essentially of SEQ ID NO:940. In some aspects, the antibody comprises a V_{H} sequence comprising, consisting of, or consisting essentially of SEQ ID NO: 941. In some aspects, the antibody comprises a V_{H} sequence comprising, consisting of, or consisting essentially of SEQ ID NO:942. In some aspects, the antibody comprises a V_{H} sequence comprising, consisting of, or consisting essentially of SEQ ID NO:943. In some aspects, the antibody comprises a V_{H} sequence comprising, consisting of, or consisting essentially of SEQ ID NO:944. In some aspects, the antibody comprises a V_{H} sequence comprising, consisting of, or consisting essentially of SEQ ID NO:945. In some aspects, the antibody comprises a V_{H} sequence comprising, consisting of, or consisting essentially of SEQ ID NO:946. In some aspects, the antibody comprises a V_{H} sequence comprising, consisting of, or consisting essentially of SEQ ID NO:947. In some aspects, the antibody comprises a V_{H} sequence comprising, consisting of, or consisting essentially of SEQ ID NO: 948. In some aspects, the antibody comprises a V_{H} sequence comprising, consisting of, or consisting essentially of SEQ ID NO:949.

In some aspects, the antibody comprises a V_{H} sequence comprising, consisting of, or consisting essentially of SEQ ID NO:950. In some aspects, the antibody comprises a V_{H} sequence comprising, consisting of, or consisting essentially of SEQ ID NO:951. In some aspects, the antibody comprises a V_{H} sequence comprising, consisting of, or consisting essentially of SEQ ID NO:952. In some aspects, the antibody comprises a V_{H} sequence comprising, consisting of, or consisting essentially of SEQ ID NO:953. In some aspects, the antibody comprises a V_{H} sequence comprising, consisting of, or consisting essentially of SEQ ID NO:954. In some aspects, the antibody comprises a V_{H} sequence comprising, consisting of, or consisting essentially of SEQ ID NO:955. In some aspects, the antibody comprises a V_{H} sequence comprising, consisting of, or consisting essentially of SEQ ID NO: 956. In some aspects, the antibody comprises a V_{H} sequence comprising, consisting of, or consisting essentially of SEQ ID NO:957. In some aspects, the antibody comprises a V_{H} sequence comprising, consisting of, or consisting essentially of SEQ ID NO:958. In some aspects, the antibody comprises a V_{H} sequence comprising, consisting of, or consisting essentially of SEQ ID NO:959. In some aspects, the antibody comprises a V_{H} sequence comprising, consisting of, or consisting essentially of SEQ ID NO:960. In some aspects, the antibody comprises a V_{H} sequence comprising, consisting of, or consisting essentially of SEQ ID NO:961. In some aspects, the antibody comprises a V_{H} sequence comprising, consisting of, or consisting essentially of SEQ ID NO:962. In some aspects, the antibody comprises a V_{H} sequence comprising, consisting of, or consisting essentially of SEQ ID NO: 963. In some aspects, the antibody comprises a V_{H} sequence comprising, consisting of, or consisting essentially of SEQ ID NO:964. In some aspects, the antibody comprises a V_{H} sequence comprising, consisting of, or consisting essentially of SEQ ID NO:965. In some aspects, the antibody comprises a V_{H} sequence comprising, consisting of, or consisting essentially of SEQ ID NO:966. In some aspects, the antibody comprises a V_{H} sequence comprising, consisting of, or consisting essentially of SEQ ID NO:967. In some aspects, the antibody comprises a V_{H} sequence comprising, consisting of, or consisting essentially of SEQ ID NO:968. In some aspects, the antibody comprises a V_{H} sequence comprising, consisting of, or consisting essentially of SEQ ID NO:969. In some aspects, the antibody comprises a V_{H} sequence comprising, consisting of, or consisting essentially of SEQ ID NO: 970. In some aspects, the antibody comprises a V_{H} sequence comprising, consisting of, or consisting essentially of SEQ ID NO:971. In some aspects, the antibody comprises a V_{H} sequence comprising, consisting of, or consisting essentially of SEQ ID NO:972. In some aspects, the antibody comprises a V_{H} sequence comprising, consisting of, or consisting essentially of SEQ ID NO:973. In some aspects, the antibody comprises a V_{H} sequence comprising, consisting of, or consisting essentially of SEQ ID NO:974. In some aspects, the antibody comprises a V_{H} sequence comprising, consisting of, or consisting essentially of SEQ ID NO:975. In some aspects, the antibody comprises a V_{H} sequence comprising, consisting of, or consisting essentially of SEQ ID NO:976. In some aspects, the antibody comprises a V_{H} sequence comprising, consisting of, or consisting essentially of SEQ ID NO: 977. In some aspects, the antibody comprises a V_{H} sequence comprising, consisting of, or consisting essentially of SEQ ID NO:978. In some aspects, the antibody comprises a V_{H} sequence comprising, consisting of, or consisting essentially of SEQ ID NO:979. In some aspects, the antibody comprises a V_{H} sequence comprising, consisting of, or consisting essentially of SEQ ID NO:980. In some aspects, the antibody comprises a V_{H} sequence comprising, consisting of, or consisting essentially of SEQ ID NO:981. In some aspects, the antibody comprises a V_{H} sequence comprising, consisting of, or consisting essentially of SEQ ID NO:982. In some aspects, the antibody comprises a V_{H} sequence comprising, consisting of, or consisting essentially of SEQ ID NO:983. In some aspects, the antibody comprises a V_{H} sequence comprising, consisting of, or consisting essentially of SEQ ID NO: 984. In some aspects, the antibody comprises a V_{H} sequence comprising, consisting of, or consisting essentially of SEQ ID NO:985. In some aspects, the antibody comprises a V_{H} sequence comprising, consisting of, or consisting essentially of SEQ ID NO:986. In some aspects, the antibody comprises a V_{H} sequence comprising, consisting of, or consisting essentially of SEQ ID NO:987. In some aspects, the antibody comprises a V_{H} sequence comprising, consisting of, or consisting essentially of SEQ ID NO:988. In some aspects, the antibody comprises a V_{H} sequence comprising, consisting of, or consisting essentially of SEQ ID NO:989. In some aspects, the antibody comprises a V_{H} sequence comprising, consisting of, or consisting essentially of SEQ ID NO:990. In some aspects, the antibody comprises a V_{H} sequence comprising, consisting of, or consisting essentially of SEQ ID NO: 991. In some aspects, the antibody comprises a V_{H} sequence comprising, consisting of, or consisting essentially of SEQ ID NO:992. In some aspects, the antibody comprises a V_{H} sequence comprising, consisting of, or consisting essentially of SEQ ID NO:993. In some aspects, the antibody comprises a V_{H} sequence comprising, consisting of, or consisting essentially of SEQ ID NO:994. In some aspects, the antibody comprises a V_{H} sequence comprising, consisting of, or consisting essentially of SEQ ID NO:995. In some aspects, the antibody comprises a V_{H} sequence comprising, consisting of, or consisting essentially of SEQ ID NO:996. In some aspects, the antibody comprises a V_{H} sequence comprising, consisting of, or consisting essentially of SEQ ID NO:997. In some aspects, the antibody comprises a V_{H} sequence comprising, consisting of, or consisting essentially of SEQ ID NO: 998. In some aspects, the antibody comprises a V_{H} sequence comprising, consisting of, or consisting essentially of SEQ ID NO:999.

In some aspects, the antibody comprises a V_{H} sequence comprising, consisting of, or consisting essentially of SEQ ID NO:1000. In some aspects, the antibody comprises a V_{H} sequence comprising, consisting of, or consisting essentially of SEQ ID NO:1001. In some aspects, the antibody comprises a V_{H} sequence comprising, consisting of, or consisting essentially of SEQ ID NO:1002. In some aspects, the antibody comprises a V_{H} sequence comprising, consisting of, or consisting essentially of SEQ ID NO:1003. In some aspects, the antibody comprises a V_{H} sequence comprising, consisting of, or consisting essentially of SEQ ID NO:1004. In some aspects, the antibody comprises a V_{H} sequence comprising, consisting of, or consisting essentially of SEQ ID NO:1005. In some aspects, the antibody comprises a V_{H} sequence comprising, consisting of, or consisting essentially of SEQ ID NO:1006. In some aspects, the antibody comprises a V_{H} sequence comprising, consisting of, or consisting essentially of SEQ ID NO: 1007. In some aspects, the antibody comprises a V_{H} sequence comprising, consisting of, or consisting essentially of SEQ ID NO: 1008. In some aspects, the antibody comprises a V_{H} sequence comprising, consisting of, or consisting essentially of SEQ ID NO:1009. In some aspects, the antibody comprises a V_{H} sequence comprising, consisting of, or consisting essentially of SEQ ID NO:1010. In some aspects, the antibody comprises a V_{H} sequence comprising, consisting of, or consisting essentially of SEQ ID NO:1011. In some aspects, the antibody comprises a V_{H} sequence comprising, consisting of, or consisting essentially of SEQ ID NO:1012. In some aspects, the antibody comprises a V_{H} sequence comprising, consisting of, or consisting essentially of SEQ ID NO:1013. In some aspects, the antibody comprises a V_{H} sequence comprising, consisting of, or consisting essentially of SEQ ID NO:1014. In some aspects, the antibody comprises a V_{H} sequence comprising, consisting of, or consisting essentially of SEQ ID NO:1015. In some aspects, the antibody comprises a V_{H} sequence comprising, consisting of, or consisting essentially of SEQ ID NO:1016. In some aspects, the antibody comprises a V_{H} sequence comprising, consisting of, or consisting essentially of SEQ ID NO: 1017. In some aspects, the antibody comprises a V_{H} sequence comprising, consisting of, or consisting essentially of SEQ ID NO:1018. In some aspects, the antibody comprises a V_{H} sequence comprising, consisting of, or consisting essentially of SEQ ID NO:1019. In some aspects, the antibody comprises a V_{H} sequence comprising, consisting of, or consisting essentially of SEQ ID NO:1020.

### 5.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.4. CDR-L3 Sequences

In some embodiments, the antibody comprises a CDR-L3 sequence comprising, consisting of, or consisting essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 1021-1026.

In some embodiments, the antibody comprises a CDR-L3 sequence comprising, consisting of, or consisting essentially of a sequence selected from SEQ ID NOs: 849-853. In some aspects, the antibody comprises a CDR-L3 sequence comprising, consisting of, or consisting essentially of SEQ ID NO: 849. In some aspects, the antibody comprises a CDR-L3 sequence comprising, consisting of, or consisting essentially of SEQ ID NO: 850. In some aspects, the antibody comprises a CDR-L3 sequence comprising, consisting of, or consisting essentially of SEQ ID NO: 851. In some aspects, the antibody comprises a CDR-L3 sequence comprising, consisting of, or consisting essentially of SEQ ID NO: 852. In some aspects, the antibody comprises a CDR-L3 sequence comprising, consisting of, or consisting essentially of SEQ ID NO: 853.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 5.5. V_{L} Sequences Comprising Illustrative CDRs

In some embodiments, the antibody comprises a V_{L} sequence comprising one or more CDR-L sequences comprising, consisting of, or consisting essentially of one or more illustrative CDR-L sequences provided in this disclosure, and variants thereof.

### 5.5.1. CDR-L3

In some embodiments, the antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of a V_{L} sequence provided in SEQ ID NOs: 1021-1026.

In some embodiments, the antibody comprises a V_{L} sequence comprising a CDR-L3 sequence comprising, consisting of, or consisting essentially of a sequence selected from SEQ ID NOs: 849-853. In some aspects, the antibody comprises a CDR-L3 sequence comprising, consisting of, or consisting essentially of SEQ ID NO: 849. In some aspects, the antibody comprises a CDR-L3 sequence comprising, consisting of, or consisting essentially of SEQ ID NO: 850. In some aspects, the antibody comprises a CDR-L3 sequence comprising, consisting of, or consisting essentially of SEQ ID NO: 851. In some aspects, the antibody comprises a CDR-L3 sequence comprising, consisting of, or consisting essentially of SEQ ID NO: 852. In some aspects, the antibody comprises a CDR-L3 sequence comprising, consisting of, or consisting essentially of SEQ ID NO: 853.

### 5.5.2. CDR-L2

In some embodiments, the antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of a V_{L} sequence provided in SEQ ID NOs: 1021-1026.

In some embodiments, the antibody comprises a V_{L} sequence comprising a CDR-L2 sequence comprising, consisting of, or consisting essentially of a sequence selected from SEQ ID NOs: 844-848. In some aspects, the antibody comprises a V_{L} sequence comprising a CDR-L2 sequence comprising, consisting of, or consisting essentially of SEQ ID NO: 844. In some aspects, the antibody comprises a V_{L} sequence comprising a CDR-L2 sequence comprising, consisting of, or consisting essentially of SEQ ID NO: 845. In some aspects, the antibody comprises a V_{L} sequence comprising a CDR-L2 sequence comprising, consisting of, or consisting essentially of SEQ ID NO: 846. In some aspects, the antibody comprises a V_{L} sequence comprising a CDR-L2 sequence comprising, consisting of, or consisting essentially of SEQ ID NO: 847. In some aspects, the antibody comprises a V_{L} sequence comprising a CDR-L2 sequence comprising, consisting of, or consisting essentially of SEQ ID NO: 848.

### 5.5.3. CDR-L1

In some embodiments, the antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of a V_{L} sequence provided in SEQ ID NOs: 1021-1026.

In some embodiments, the antibody comprises a V_{L} sequence comprising a CDR-L1 sequence comprising, consisting of, or consisting essentially of a sequence selected from SEQ ID NOs: 839-843. In some aspects, the antibody comprises a V_{L} sequence comprising a CDR-L1 sequence comprising, consisting of, or consisting essentially of SEQ ID NO: 839. In some aspects, the antibody comprises a V_{L} sequence comprising a CDR-L1 sequence comprising, consisting of, or consisting essentially of SEQ ID NO: 840. In some aspects, the antibody comprises a V_{L} sequence comprising a CDR-L1 sequence comprising, consisting of, or consisting essentially of SEQ ID NO: 841. In some aspects, the antibody comprises a V_{L} sequence comprising a CDR-L1 sequence comprising, consisting of, or consisting essentially of SEQ ID NO: 842. In some aspects, the antibody comprises a V_{L} sequence comprising a CDR-L1 sequence comprising, consisting of, or consisting essentially of SEQ ID NO: 843.

### 5.5.4. CDR-L3+CDR-L2

In some embodiments, the antibody comprises a V_{L} sequence comprising a CDR-L3 sequence comprising, consisting of, or consisting essentially of a sequence selected from SEQ ID NOs: 849-853 and a CDR-L2 sequence comprising, consisting of, or consisting essentially of a sequence selected from SEQ ID NOs: 844-848. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 1021-1026.

### 5.5.5. CDR-L3+CDR-L1

In some embodiments, the antibody comprises a V_{L} sequence comprising a CDR-L3 sequence comprising, consisting of, or consisting essentially of a sequence selected from SEQ ID NOs: 849-853 and a CDR-L1 sequence comprising, consisting of, or consisting essentially of a sequence selected from SEQ ID NOs: 839-843. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 1021-1026.

### 5.5.6. CDR-L1+CDR-L2

In some embodiments, the antibody comprises a V_{L} sequence comprising a CDR-L1 sequence comprising, consisting of, or consisting essentially of a sequence selected from SEQ ID NOs: 839-843 and a CDR-L2 sequence comprising, consisting of, or consisting essentially of a sequence selected from SEQ ID NOs: 844-848. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 1021-1026.

### 5.5.7. CDR-L1+CDR-L2+CDR-L3

In some embodiments, the antibody comprises a V_{L} sequence comprising a CDR-L1 sequence comprising, consisting of, or consisting essentially of a sequence selected from SEQ ID NOs: 839-843, a CDR-L2 sequence comprising, consisting of, or consisting essentially of a sequence selected from SEQ ID NOs: 844-848, and a CDR-L3 sequence comprising, consisting of, or consisting essentially of a sequence selected from SEQ ID NOs: 849-853. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 1021-1026.

### 5.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, 95%, 96%, 97%, 98%, or 99% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, 95%, 96%, 97%, 98%, or 99% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 5.6. V_{L} Sequences

In some embodiments, the antibody comprises, consists of, or consists essentially of a V_{L} sequence provided in SEQ ID NOs: 1021-1026.

In some embodiments, the antibody comprises a V_{L} sequence comprising, consisting of, or consisting essentially of a sequence selected from SEQ ID NOs: 1021-1026. In some aspects, the antibody comprises a V_{L} sequence comprising, consisting of, or consisting essentially of SEQ ID NO: 1021. In some aspects, the antibody comprises a V_{L} sequence comprising, consisting of, or consisting essentially of SEQ ID NO: 1022. In some aspects, the antibody comprises a V_{L} sequence comprising, consisting of, or consisting essentially of SEQ ID NO: 1023. In some aspects, the antibody comprises a V_{L} sequence comprising, consisting of, or consisting essentially of SEQ ID NO: 1024. In some aspects, the antibody comprises a V_{L} sequence comprising, consisting of, or consisting essentially of SEQ ID NO: 1025. In some aspects, the antibody comprises a V_{L} sequence comprising, consisting of, or consisting essentially of SEQ ID NO: 1026.

### 5.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.7. Pairs

### 5.7.1. CDR-H3-CDR-L3 Pairs

In some embodiments, the antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of any one of SEQ ID NOs: 672-838, and the CDR-L3 sequence is a CDR-L3 sequence comprising, consisting of, or consisting essentially of any one of SEQ ID NOs: 849-853.

In some aspects, the CDR-H3-CDR-L3 pairs are selected from SEQ ID NO:357 and SEQ ID NO: 849; SEQ ID NO:358 and SEQ ID NO:849; SEQ ID NO:359 and SEQ ID NO: 849; SEQ ID NO:360 and SEQ ID NO:849; SEQ ID NO:361 and SEQ ID NO:849; SEQ ID NO:362 and SEQ ID NO: 849; SEQ ID NO:363 and SEQ ID NO:849; SEQ ID NO: 364 and SEQ ID NO: 849; SEQ ID NO:365 and SEQ ID NO: 849; SEQ ID NO:366 and SEQ ID NO: 849; SEQ ID NO:367 and SEQ ID NO:849; SEQ ID NO:368 and SEQ ID NO: 849; SEQ ID NO:369 and SEQ ID NO:849; SEQ ID NO:370 and SEQ ID NO:849; SEQ ID NO:371 and SEQ ID NO:849; SEQ ID NO:372 and SEQ ID NO:849; SEQ ID NO: 373 and SEQ ID NO: 849; SEQ ID NO:374 and SEQ ID NO: 849; SEQ ID NO:375 and SEQ ID NO: 849; SEQ ID NO:376 and SEQ ID NO:849; SEQ ID NO:377 and SEQ ID NO: 849; SEQ ID NO:378 and SEQ ID NO:849; SEQ ID NO:379 and SEQ ID NO:849; SEQ ID NO:380 and SEQ ID NO: 849; SEQ ID NO:381 and SEQ ID NO:849; SEQ ID NO: 382 and SEQ ID NO: 849; SEQ ID NO:383 and SEQ ID NO:849; SEQ ID NO:384 and SEQ ID NO:849; SEQ ID NO:385 and SEQ ID NO: 849; SEQ ID NO:386 and SEQ ID NO: 849; SEQ ID NO:387 and SEQ ID NO: 849; SEQ ID NO:388 and SEQ ID NO:849; SEQ ID NO:389 and SEQ ID NO: 849; SEQ ID NO:390 and SEQ ID NO:849; SEQ ID NO: 391 and SEQ ID NO: 849; SEQ ID NO:392 and SEQ ID NO:849; SEQ ID NO:393 and SEQ ID NO: 849; SEQ ID NO:394 and SEQ ID NO: 849; SEQ ID NO:395 and SEQ ID NO: 849; SEQ ID NO:396 and SEQ ID NO: 849; SEQ ID NO:397 and SEQ ID NO:849; SEQ ID NO:398 and SEQ ID NO: 849; SEQ ID NO:399 and SEQ ID NO: 849; SEQ ID NO: 400 and SEQ ID NO: 849; SEQ ID NO:401 and SEQ ID NO: 849; SEQ ID NO:402 and SEQ ID NO: 849; SEQ ID NO:403 and SEQ ID NO:849; SEQ ID NO:404 and SEQ ID NO: 849; SEQ ID NO:405 and SEQ ID NO: 849; SEQ ID NO:406 and SEQ ID NO:849; SEQ ID NO:407 and SEQ ID NO:849; SEQ ID NO:408 and SEQ ID NO:849; SEQ ID NO: 409 and SEQ ID NO: 849; SEQ ID NO:410 and SEQ ID NO: 849; SEQ ID NO:411 and SEQ ID NO: 849; SEQ ID NO:412 and SEQ ID NO:849; SEQ ID NO:413 and SEQ ID NO: 849; SEQ ID NO:414 and SEQ ID NO: 849; SEQ ID NO:415 and SEQ ID NO:849; SEQ ID NO:416 and SEQ ID NO: 849; SEQ ID NO:417 and SEQ ID NO: 849; SEQ ID NO: 418 and SEQ ID NO: 849; SEQ ID NO:419 and SEQ ID NO:849; SEQ ID NO:420 and SEQ ID NO: 849; SEQ ID NO:421 and SEQ ID NO:849; SEQ ID NO:422 and SEQ ID NO: 849; SEQ ID NO:423 and SEQ ID NO: 849; SEQ ID NO:424 and SEQ ID NO:849; SEQ ID NO:425 and SEQ ID NO:849; SEQ ID NO:426 and SEQ ID NO:849; SEQ ID NO: 427 and SEQ ID NO: 849; SEQ ID NO: 428 and SEQ ID NO: 849; SEQ ID NO:429 and SEQ ID NO: 849; SEQ ID NO:430 and SEQ ID NO:849; SEQ ID NO:431 and SEQ ID NO: 849; SEQ ID NO:432 and SEQ ID NO:849; SEQ ID NO:433 and SEQ ID NO:849; SEQ ID NO:434 and SEQ ID NO: 849; SEQ ID NO:435 and SEQ ID NO:849; SEQ ID NO: 436 and SEQ ID NO: 849; SEQ ID NO:437 and SEQ ID NO: 849; SEQ ID NO:438 and SEQ ID NO: 849; SEQ ID NO:439 and SEQ ID NO:849; SEQ ID NO:440 and SEQ ID NO: 849; SEQ ID NO:441 and SEQ ID NO: 849; SEQ ID NO:442 and SEQ ID NO:849; SEQ ID NO:443 and SEQ ID NO:849; SEQ ID NO:444 and SEQ ID NO:849; SEQ ID NO: 445 and SEQ ID NO: 849; SEQ ID NO:446 and SEQ ID NO:849; SEQ ID NO:447 and SEQ ID NO: 849; SEQ ID NO:448 and SEQ ID NO:849; SEQ ID NO:449 and SEQ ID NO: 849; SEQ ID NO:450 and SEQ ID NO:849; SEQ ID NO:451 and SEQ ID NO:849; SEQ ID NO:452 and SEQ ID NO: 849; SEQ ID NO:453 and SEQ ID NO:849; SEQ ID NO: 454 and SEQ ID NO: 849; SEQ ID NO:455 and SEQ ID NO:849; SEQ ID NO:456 and SEQ ID NO: 849; SEQ ID NO:457 and SEQ ID NO:849; SEQ ID NO:458 and SEQ ID NO: 849; SEQ ID NO:459 and SEQ ID NO: 849; SEQ ID NO:460 and SEQ ID NO:849; SEQ ID NO:461 and SEQ ID NO: 849; SEQ ID NO:462 and SEQ ID NO: 849; SEQ ID NO: 463 and SEQ ID NO: 849; SEQ ID NO:464 and SEQ ID NO: 849; SEQ ID NO:465 and SEQ ID NO: 849; SEQ ID NO:466 and SEQ ID NO:849; SEQ ID NO:467 and SEQ ID NO: 849; SEQ ID NO:468 and SEQ ID NO:849; SEQ ID NO:469 and SEQ ID NO:849; SEQ ID NO:470 and SEQ ID NO:849; SEQ ID NO:471 and SEQ ID NO:849; SEQ ID NO: 472 and SEQ ID NO: 849; SEQ ID NO:473 and SEQ ID NO: 849; SEQ ID NO:474 and SEQ ID NO: 849; SEQ ID NO:475 and SEQ ID NO:849; SEQ ID NO:476 and SEQ ID NO: 849; SEQ ID NO:477 and SEQ ID NO:849; SEQ ID NO:478 and SEQ ID NO:849; SEQ ID NO:479 and SEQ ID NO:849; SEQ ID NO:480 and SEQ ID NO:849; SEQ ID NO: 481 and SEQ ID NO: 849; SEQ ID NO: 482 and SEQ ID NO: 849; SEQ ID NO:483 and SEQ ID NO: 849; SEQ ID NO:484 and SEQ ID NO:849; SEQ ID NO:485 and SEQ ID NO: 849; SEQ ID NO: 486 and SEQ ID NO: 849; SEQ ID NO:487 and SEQ ID NO:849; SEQ ID NO: 488 and SEQ ID NO: 849; SEQ ID NO:489 and SEQ ID NO:849; SEQ ID NO: 490 and SEQ ID NO: 849; SEQ ID NO:491 and SEQ ID NO: 849; SEQ ID NO:492 and SEQ ID NO: 849; SEQ ID NO:493 and SEQ ID NO:849; SEQ ID NO:494 and SEQ ID NO: 849; SEQ ID NO:495 and SEQ ID NO: 849; SEQ ID NO:496 and SEQ ID NO:849; SEQ ID NO: 497 and SEQ ID NO:849; SEQ ID NO:498 and SEQ ID NO:849; SEQ ID NO: 499 and SEQ ID NO: 849; SEQ ID NO:500 and SEQ ID NO:849; SEQ ID NO:501 and SEQ ID NO: 849; SEQ ID NO:502 and SEQ ID NO:849; SEQ ID NO:503 and SEQ ID NO: 849; SEQ ID NO:504 and SEQ ID NO: 849; SEQ ID NO:505 and SEQ ID NO:849; SEQ ID NO:506 and SEQ ID NO:849; SEQ ID NO:507 and SEQ ID NO:849; SEQ ID NO: 508 and SEQ ID NO: 849; SEQ ID NO:509 and SEQ ID NO: 849; SEQ ID NO:510 and SEQ ID NO:849; SEQ ID NO:511 and SEQ ID NO:849; SEQ ID NO:512 and SEQ ID NO: 849; SEQ ID NO:513 and SEQ ID NO: 849; SEQ ID NO:514 and SEQ ID NO:849; SEQ ID NO:515 and SEQ ID NO: 849; SEQ ID NO:516 and SEQ ID NO: 849; SEQ ID NO: 517 and SEQ ID NO: 849; SEQ ID NO:518 and SEQ ID NO:849; SEQ ID NO:519 and SEQ ID NO: 849; SEQ ID NO:520 and SEQ ID NO:849; SEQ ID NO:521 and SEQ ID NO: 849; SEQ ID NO:522 and SEQ ID NO:849; and SEQ ID NO:523 and SEQ ID NO: 849.

In some aspects, the CDR-H3-CDR-L3 pairs are selected from SEQ ID NO:357 and SEQ ID NO:850; SEQ ID NO:358 and SEQ ID NO:850; SEQ ID NO:359 and SEQ ID NO: 850; SEQ ID NO:360 and SEQ ID NO: 850; SEQ ID NO:361 and SEQ ID NO:850; SEQ ID NO:362 and SEQ ID NO:850; SEQ ID NO:363 and SEQ ID NO:850; SEQ ID NO: 364 and SEQ ID NO:850; SEQ ID NO:365 and SEQ ID NO:850; SEQ ID NO:366 and SEQ ID NO:850; SEQ ID NO:367 and SEQ ID NO:850; SEQ ID NO:368 and SEQ ID NO: 850; SEQ ID NO:369 and SEQ ID NO:850; SEQ ID NO:370 and SEQ ID NO:850; SEQ ID NO:371 and SEQ ID NO:850; SEQ ID NO:372 and SEQ ID NO:850; SEQ ID NO: 373 and SEQ ID NO:850; SEQ ID NO:374 and SEQ ID NO:850; SEQ ID NO:375 and SEQ ID NO:850; SEQ ID NO:376 and SEQ ID NO:850; SEQ ID NO:377 and SEQ ID NO: 850; SEQ ID NO:378 and SEQ ID NO:850; SEQ ID NO:379 and SEQ ID NO:850; SEQ ID NO:380 and SEQ ID NO:850; SEQ ID NO:381 and SEQ ID NO:850; SEQ ID NO: 382 and SEQ ID NO: 850; SEQ ID NO:383 and SEQ ID NO:850; SEQ ID NO:384 and SEQ ID NO: 850; SEQ ID NO:385 and SEQ ID NO:850; SEQ ID NO:386 and SEQ ID NO: 850; SEQ ID NO:387 and SEQ ID NO:850; SEQ ID NO:388 and SEQ ID NO:850; SEQ ID NO:389 and SEQ ID NO:850; SEQ ID NO:390 and SEQ ID NO:850; SEQ ID NO: 391 and SEQ ID NO:850; SEQ ID NO:392 and SEQ ID NO:850; SEQ ID NO:393 and SEQ ID NO:850; SEQ ID NO:394 and SEQ ID NO:850; SEQ ID NO:395 and SEQ ID NO: 850; SEQ ID NO:396 and SEQ ID NO:850; SEQ ID NO:397 and SEQ ID NO:850; SEQ ID NO:398 and SEQ ID NO:850; SEQ ID NO:399 and SEQ ID NO:850; SEQ ID NO: 400 and SEQ ID NO: 850; SEQ ID NO:401 and SEQ ID NO: 850; SEQ ID NO:402 and SEQ ID NO:850; SEQ ID NO:403 and SEQ ID NO:850; SEQ ID NO:404 and SEQ ID NO: 850; SEQ ID NO:405 and SEQ ID NO: 850; SEQ ID NO:406 and SEQ ID NO:850; SEQ ID NO:407 and SEQ ID NO:850; SEQ ID NO:408 and SEQ ID NO:850; SEQ ID NO: 409 and SEQ ID NO:850; SEQ ID NO:410 and SEQ ID NO:850; SEQ ID NO:411 and SEQ ID NO:850; SEQ ID NO:412 and SEQ ID NO:850; SEQ ID NO:413 and SEQ ID NO: 850; SEQ ID NO:414 and SEQ ID NO:850; SEQ ID NO:415 and SEQ ID NO:850; SEQ ID NO:416 and SEQ ID NO:850; SEQ ID NO:417 and SEQ ID NO:850; SEQ ID NO: 418 and SEQ ID NO: 850; SEQ ID NO:419 and SEQ ID NO:850; SEQ ID NO:420 and SEQ ID NO:850; SEQ ID NO:421 and SEQ ID NO:850; SEQ ID NO:422 and SEQ ID NO: 850; SEQ ID NO:423 and SEQ ID NO:850; SEQ ID NO:424 and SEQ ID NO:850; SEQ ID NO:425 and SEQ ID NO:850; SEQ ID NO:426 and SEQ ID NO:850; SEQ ID NO: 427 and SEQ ID NO: 850; SEQ ID NO:428 and SEQ ID NO:850; SEQ ID NO:429 and SEQ ID NO: 850; SEQ ID NO:430 and SEQ ID NO:850; SEQ ID NO:431 and SEQ ID NO: 850; SEQ ID NO:432 and SEQ ID NO: 850; SEQ ID NO:433 and SEQ ID NO:850; SEQ ID NO:434 and SEQ ID NO:850; SEQ ID NO:435 and SEQ ID NO:850; SEQ ID NO: 436 and SEQ ID NO: 850; SEQ ID NO:437 and SEQ ID NO: 850; SEQ ID NO:438 and SEQ ID NO:850; SEQ ID NO:439 and SEQ ID NO:850; SEQ ID NO:440 and SEQ ID NO: 850; SEQ ID NO:441 and SEQ ID NO:850; SEQ ID NO:442 and SEQ ID NO:850; SEQ ID NO:443 and SEQ ID NO: 850; SEQ ID NO:444 and SEQ ID NO:850; SEQ ID NO: 445 and SEQ ID NO: 850; SEQ ID NO:446 and SEQ ID NO: 850; SEQ ID NO:447 and SEQ ID NO: 850; SEQ ID NO:448 and SEQ ID NO:850; SEQ ID NO:449 and SEQ ID NO: 850; SEQ ID NO:450 and SEQ ID NO: 850; SEQ ID NO:451 and SEQ ID NO:850; SEQ ID NO:452 and SEQ ID NO: 850; SEQ ID NO:453 and SEQ ID NO:850; SEQ ID NO: 454 and SEQ ID NO: 850; SEQ ID NO:455 and SEQ ID NO: 850; SEQ ID NO:456 and SEQ ID NO:850; SEQ ID NO:457 and SEQ ID NO:850; SEQ ID NO:458 and SEQ ID NO: 850; SEQ ID NO:459 and SEQ ID NO:850; SEQ ID NO:460 and SEQ ID NO:850; SEQ ID NO:461 and SEQ ID NO:850; SEQ ID NO:462 and SEQ ID NO:850; SEQ ID NO: 463 and SEQ ID NO: 850; SEQ ID NO:464 and SEQ ID NO:850; SEQ ID NO:465 and SEQ ID NO: 850; SEQ ID NO:466 and SEQ ID NO:850; SEQ ID NO:467 and SEQ ID NO: 850; SEQ ID NO:468 and SEQ ID NO: 850; SEQ ID NO:469 and SEQ ID NO:850; SEQ ID NO:470 and SEQ ID NO:850; SEQ ID NO:471 and SEQ ID NO:850; SEQ ID NO: 472 and SEQ ID NO: 850; SEQ ID NO:473 and SEQ ID NO: 850; SEQ ID NO:474 and SEQ ID NO: 850; SEQ ID NO:475 and SEQ ID NO: 850; SEQ ID NO:476 and SEQ ID NO: 850; SEQ ID NO:477 and SEQ ID NO:850; SEQ ID NO:478 and SEQ ID NO:850; SEQ ID NO:479 and SEQ ID NO:850; SEQ ID NO:480 and SEQ ID NO:850; SEQ ID NO: 481 and SEQ ID NO: 850; SEQ ID NO:482 and SEQ ID NO: 850; SEQ ID NO:483 and SEQ ID NO:850; SEQ ID NO:484 and SEQ ID NO:850; SEQ ID NO:485 and SEQ ID NO: 850; SEQ ID NO:486 and SEQ ID NO:850; SEQ ID NO:487 and SEQ ID NO:850; SEQ ID NO:488 and SEQ ID NO:850; SEQ ID NO:489 and SEQ ID NO:850; SEQ ID NO: 490 and SEQ ID NO: 850; SEQ ID NO:491 and SEQ ID NO: 850; SEQ ID NO:492 and SEQ ID NO:850; SEQ ID NO:493 and SEQ ID NO:850; SEQ ID NO:494 and SEQ ID NO: 850; SEQ ID NO:495 and SEQ ID NO:850; SEQ ID NO:496 and SEQ ID NO:850; SEQ ID NO:497 and SEQ ID NO:850; SEQ ID NO:498 and SEQ ID NO:850; SEQ ID NO: 499 and SEQ ID NO: 850; SEQ ID NO:500 and SEQ ID NO:850; SEQ ID NO:501 and SEQ ID NO:850; SEQ ID NO:502 and SEQ ID NO:850; SEQ ID NO:503 and SEQ ID NO: 850; SEQ ID NO:504 and SEQ ID NO:850; SEQ ID NO:505 and SEQ ID NO:850; SEQ ID NO:506 and SEQ ID NO:850; SEQ ID NO:507 and SEQ ID NO:850; SEQ ID NO: 508 and SEQ ID NO:850; SEQ ID NO:509 and SEQ ID NO:850; SEQ ID NO:510 and SEQ ID NO:850; SEQ ID NO:511 and SEQ ID NO:850; SEQ ID NO:512 and SEQ ID NO: 850; SEQ ID NO:513 and SEQ ID NO:850; SEQ ID NO:514 and SEQ ID NO:850; SEQ ID NO:515 and SEQ ID NO:850; SEQ ID NO:516 and SEQ ID NO:850; SEQ ID NO: 517 and SEQ ID NO: 850; SEQ ID NO:518 and SEQ ID NO: 850; SEQ ID NO:519 and SEQ ID NO:850; SEQ ID NO:520 and SEQ ID NO:850; SEQ ID NO:521 and SEQ ID NO: 850; SEQ ID NO:522 and SEQ ID NO:850; and SEQ ID NO:523 and SEQ ID NO: 850.

In some aspects, the CDR-H3-CDR-L3 pairs are selected from SEQ ID NO:357 and SEQ ID NO:851; SEQ ID NO:358 and SEQ ID NO:851; SEQ ID NO:359 and SEQ ID NO: 851; SEQ ID NO:360 and SEQ ID NO:851; SEQ ID NO:361 and SEQ ID NO:851; SEQ ID NO:362 and SEQ ID NO:851; SEQ ID NO:363 and SEQ ID NO:851; SEQ ID NO: 364 and SEQ ID NO: 851; SEQ ID NO:365 and SEQ ID NO:851; SEQ ID NO:366 and SEQ ID NO:851; SEQ ID NO:367 and SEQ ID NO:851; SEQ ID NO:368 and SEQ ID NO: 851; SEQ ID NO:369 and SEQ ID NO:851; SEQ ID NO:370 and SEQ ID NO:851; SEQ ID NO:371 and SEQ ID NO:851; SEQ ID NO:372 and SEQ ID NO:851; SEQ ID NO: 373 and SEQ ID NO: 851; SEQ ID NO:374 and SEQ ID NO:851; SEQ ID NO:375 and SEQ ID NO:851; SEQ ID NO:376 and SEQ ID NO:851; SEQ ID NO:377 and SEQ ID NO: 851; SEQ ID NO:378 and SEQ ID NO:851; SEQ ID NO:379 and SEQ ID NO:851; SEQ ID NO:380 and SEQ ID NO:851; SEQ ID NO:381 and SEQ ID NO:851; SEQ ID NO: 382 and SEQ ID NO:851; SEQ ID NO:383 and SEQ ID NO:851; SEQ ID NO:384 and SEQ ID NO:851; SEQ ID NO:385 and SEQ ID NO:851; SEQ ID NO:386 and SEQ ID NO: 851; SEQ ID NO:387 and SEQ ID NO:851; SEQ ID NO:388 and SEQ ID NO:851; SEQ ID NO:389 and SEQ ID NO:851; SEQ ID NO:390 and SEQ ID NO:851; SEQ ID NO: 391 and SEQ ID NO: 851; SEQ ID NO:392 and SEQ ID NO:851; SEQ ID NO:393 and SEQ ID NO:851; SEQ ID NO:394 and SEQ ID NO:851; SEQ ID NO:395 and SEQ ID NO: 851; SEQ ID NO:396 and SEQ ID NO:851; SEQ ID NO:397 and SEQ ID NO:851; SEQ ID NO:398 and SEQ ID NO:851; SEQ ID NO:399 and SEQ ID NO:851; SEQ ID NO: 400 and SEQ ID NO: 851; SEQ ID NO:401 and SEQ ID NO:851; SEQ ID NO:402 and SEQ ID NO:851; SEQ ID NO:403 and SEQ ID NO:851; SEQ ID NO:404 and SEQ ID NO: 851; SEQ ID NO:405 and SEQ ID NO:851; SEQ ID NO:406 and SEQ ID NO:851; SEQ ID NO:407 and SEQ ID NO:851; SEQ ID NO:408 and SEQ ID NO:851; SEQ ID NO: 409 and SEQ ID NO:851; SEQ ID NO:410 and SEQ ID NO:851; SEQ ID NO:411 and SEQ ID NO:851; SEQ ID NO:412 and SEQ ID NO:851; SEQ ID NO:413 and SEQ ID NO: 851; SEQ ID NO:414 and SEQ ID NO: 851; SEQ ID NO:415 and SEQ ID NO:851; SEQ ID NO:416 and SEQ ID NO:851; SEQ ID NO:417 and SEQ ID NO:851; SEQ ID NO: 418 and SEQ ID NO:851; SEQ ID NO:419 and SEQ ID NO:851; SEQ ID NO:420 and SEQ ID NO:851; SEQ ID NO:421 and SEQ ID NO:851; SEQ ID NO:422 and SEQ ID NO: 851; SEQ ID NO:423 and SEQ ID NO:851; SEQ ID NO:424 and SEQ ID NO:851; SEQ ID NO:425 and SEQ ID NO:851; SEQ ID NO:426 and SEQ ID NO:851; SEQ ID NO: 427 and SEQ ID NO: 851; SEQ ID NO:428 and SEQ ID NO:851; SEQ ID NO:429 and SEQ ID NO:851; SEQ ID NO:430 and SEQ ID NO:851; SEQ ID NO:431 and SEQ ID NO: 851; SEQ ID NO:432 and SEQ ID NO:851; SEQ ID NO:433 and SEQ ID NO:851; SEQ ID NO:434 and SEQ ID NO: 851; SEQ ID NO:435 and SEQ ID NO:851; SEQ ID NO: 436 and SEQ ID NO: 851; SEQ ID NO:437 and SEQ ID NO:851; SEQ ID NO:438 and SEQ ID NO: 851; SEQ ID NO:439 and SEQ ID NO:851; SEQ ID NO:440 and SEQ ID NO: 851; SEQ ID NO:441 and SEQ ID NO:851; SEQ ID NO:442 and SEQ ID NO:851; SEQ ID NO:443 and SEQ ID NO: 851; SEQ ID NO:444 and SEQ ID NO:851; SEQ ID NO: 445 and SEQ ID NO:851; SEQ ID NO:446 and SEQ ID NO:851; SEQ ID NO:447 and SEQ ID NO:851; SEQ ID NO:448 and SEQ ID NO:851; SEQ ID NO:449 and SEQ ID NO: 851; SEQ ID NO:450 and SEQ ID NO:851; SEQ ID NO:451 and SEQ ID NO:851; SEQ ID NO:452 and SEQ ID NO:851; SEQ ID NO:453 and SEQ ID NO:851; SEQ ID NO: 454 and SEQ ID NO: 851; SEQ ID NO:455 and SEQ ID NO:851; SEQ ID NO:456 and SEQ ID NO:851; SEQ ID NO:457 and SEQ ID NO:851; SEQ ID NO:458 and SEQ ID NO: 851; SEQ ID NO:459 and SEQ ID NO:851; SEQ ID NO:460 and SEQ ID NO:851; SEQ ID NO:461 and SEQ ID NO:851; SEQ ID NO:462 and SEQ ID NO:851; SEQ ID NO: 463 and SEQ ID NO:851; SEQ ID NO:464 and SEQ ID NO:851; SEQ ID NO:465 and SEQ ID NO: 851; SEQ ID NO:466 and SEQ ID NO: 851; SEQ ID NO:467 and SEQ ID NO: 851; SEQ ID NO:468 and SEQ ID NO:851; SEQ ID NO:469 and SEQ ID NO:851; SEQ ID NO:470 and SEQ ID NO:851; SEQ ID NO:471 and SEQ ID NO:851; SEQ ID NO: 472 and SEQ ID NO: 851; SEQ ID NO:473 and SEQ ID NO:851; SEQ ID NO:474 and SEQ ID NO:851; SEQ ID NO:475 and SEQ ID NO:851; SEQ ID NO:476 and SEQ ID NO: 851; SEQ ID NO:477 and SEQ ID NO:851; SEQ ID NO:478 and SEQ ID NO:851; SEQ ID NO:479 and SEQ ID NO:851; SEQ ID NO:480 and SEQ ID NO:851; SEQ ID NO: 481 and SEQ ID NO: 851; SEQ ID NO:482 and SEQ ID NO:851; SEQ ID NO:483 and SEQ ID NO:851; SEQ ID NO:484 and SEQ ID NO:851; SEQ ID NO:485 and SEQ ID NO: 851; SEQ ID NO:486 and SEQ ID NO:851; SEQ ID NO:487 and SEQ ID NO:851; SEQ ID NO:488 and SEQ ID NO:851; SEQ ID NO:489 and SEQ ID NO:851; SEQ ID NO: 490 and SEQ ID NO: 851; SEQ ID NO:491 and SEQ ID NO: 851; SEQ ID NO:492 and SEQ ID NO: 851; SEQ ID NO:493 and SEQ ID NO:851; SEQ ID NO:494 and SEQ ID NO: 851; SEQ ID NO:495 and SEQ ID NO:851; SEQ ID NO:496 and SEQ ID NO:851; SEQ ID NO:497 and SEQ ID NO:851; SEQ ID NO:498 and SEQ ID NO:851; SEQ ID NO: 499 and SEQ ID NO:851; SEQ ID NO:500 and SEQ ID NO:851; SEQ ID NO:501 and SEQ ID NO:851; SEQ ID NO:502 and SEQ ID NO:851; SEQ ID NO:503 and SEQ ID NO: 851; SEQ ID NO:504 and SEQ ID NO:851; SEQ ID NO:505 and SEQ ID NO:851; SEQ ID NO:506 and SEQ ID NO:851; SEQ ID NO:507 and SEQ ID NO:851; SEQ ID NO: 508 and SEQ ID NO:851; SEQ ID NO:509 and SEQ ID NO:851; SEQ ID NO:510 and SEQ ID NO:851; SEQ ID NO:511 and SEQ ID NO:851; SEQ ID NO:512 and SEQ ID NO: 851; SEQ ID NO:513 and SEQ ID NO:851; SEQ ID NO:514 and SEQ ID NO:851; SEQ ID NO:515 and SEQ ID NO:851; SEQ ID NO:516 and SEQ ID NO:851; SEQ ID NO: 517 and SEQ ID NO:851; SEQ ID NO:518 and SEQ ID NO:851; SEQ ID NO:519 and SEQ ID NO:851; SEQ ID NO:520 and SEQ ID NO:851; SEQ ID NO:521 and SEQ ID NO: 851; SEQ ID NO:522 and SEQ ID NO:851; and SEQ ID NO:523 and SEQ ID NO: 851.

In some aspects, the CDR-H3-CDR-L3 pairs are selected from SEQ ID NO:357 and SEQ ID NO:852; SEQ ID NO:358 and SEQ ID NO: 852; SEQ ID NO:359 and SEQ ID NO: 852; SEQ ID NO:360 and SEQ ID NO:852; SEQ ID NO:361 and SEQ ID NO:852; SEQ ID NO:362 and SEQ ID NO:852; SEQ ID NO:363 and SEQ ID NO:852; SEQ ID NO: 364 and SEQ ID NO: 852; SEQ ID NO:365 and SEQ ID NO: 852; SEQ ID NO:366 and SEQ ID NO: 852; SEQ ID NO:367 and SEQ ID NO: 852; SEQ ID NO:368 and SEQ ID NO: 852; SEQ ID NO:369 and SEQ ID NO:852; SEQ ID NO:370 and SEQ ID NO:852; SEQ ID NO:371 and SEQ ID NO:852; SEQ ID NO:372 and SEQ ID NO:852; SEQ ID NO: 373 and SEQ ID NO: 852; SEQ ID NO:374 and SEQ ID NO:852; SEQ ID NO:375 and SEQ ID NO:852; SEQ ID NO:376 and SEQ ID NO:852; SEQ ID NO:377 and SEQ ID NO: 852; SEQ ID NO:378 and SEQ ID NO:852; SEQ ID NO:379 and SEQ ID NO:852; SEQ ID NO:380 and SEQ ID NO:852; SEQ ID NO:381 and SEQ ID NO:852; SEQ ID NO: 382 and SEQ ID NO: 852; SEQ ID NO:383 and SEQ ID NO: 852; SEQ ID NO:384 and SEQ ID NO:852; SEQ ID NO:385 and SEQ ID NO:852; SEQ ID NO:386 and SEQ ID NO: 852; SEQ ID NO:387 and SEQ ID NO:852; SEQ ID NO:388 and SEQ ID NO:852; SEQ ID NO:389 and SEQ ID NO:852; SEQ ID NO:390 and SEQ ID NO:852; SEQ ID NO: 391 and SEQ ID NO: 852; SEQ ID NO:392 and SEQ ID NO:852; SEQ ID NO:393 and SEQ ID NO:852; SEQ ID NO:394 and SEQ ID NO:852; SEQ ID NO:395 and SEQ ID NO: 852; SEQ ID NO:396 and SEQ ID NO:852; SEQ ID NO:397 and SEQ ID NO:852; SEQ ID NO:398 and SEQ ID NO:852; SEQ ID NO:399 and SEQ ID NO:852; SEQ ID NO: 400 and SEQ ID NO:852; SEQ ID NO:401 and SEQ ID NO:852; SEQ ID NO:402 and SEQ ID NO:852; SEQ ID NO:403 and SEQ ID NO: 852; SEQ ID NO:404 and SEQ ID NO: 852; SEQ ID NO:405 and SEQ ID NO: 852; SEQ ID NO:406 and SEQ ID NO:852; SEQ ID NO:407 and SEQ ID NO:852; SEQ ID NO:408 and SEQ ID NO:852; SEQ ID NO: 409 and SEQ ID NO: 852; SEQ ID NO:410 and SEQ ID NO:852; SEQ ID NO:411 and SEQ ID NO:852; SEQ ID NO:412 and SEQ ID NO: 852; SEQ ID NO:413 and SEQ ID NO: 852; SEQ ID NO:414 and SEQ ID NO:852; SEQ ID NO:415 and SEQ ID NO:852; SEQ ID NO:416 and SEQ ID NO:852; SEQ ID NO:417 and SEQ ID NO:852; SEQ ID NO: 418 and SEQ ID NO: 852; SEQ ID NO:419 and SEQ ID NO:852; SEQ ID NO:420 and SEQ ID NO:852; SEQ ID NO:421 and SEQ ID NO: 852; SEQ ID NO:422 and SEQ ID NO: 852; SEQ ID NO:423 and SEQ ID NO:852; SEQ ID NO:424 and SEQ ID NO:852; SEQ ID NO:425 and SEQ ID NO:852; SEQ ID NO:426 and SEQ ID NO:852; SEQ ID NO: 427 and SEQ ID NO: 852; SEQ ID NO:428 and SEQ ID NO:852; SEQ ID NO:429 and SEQ ID NO:852; SEQ ID NO:430 and SEQ ID NO:852; SEQ ID NO:431 and SEQ ID NO: 852; SEQ ID NO:432 and SEQ ID NO:852; SEQ ID NO:433 and SEQ ID NO:852; SEQ ID NO:434 and SEQ ID NO:852; SEQ ID NO:435 and SEQ ID NO:852; SEQ ID NO: 436 and SEQ ID NO: 852; SEQ ID NO:437 and SEQ ID NO:852; SEQ ID NO:438 and SEQ ID NO:852; SEQ ID NO:439 and SEQ ID NO:852; SEQ ID NO:440 and SEQ ID NO: 852; SEQ ID NO:441 and SEQ ID NO: 852; SEQ ID NO:442 and SEQ ID NO:852; SEQ ID NO:443 and SEQ ID NO:852; SEQ ID NO:444 and SEQ ID NO:852; SEQ ID NO: 445 and SEQ ID NO: 852; SEQ ID NO:446 and SEQ ID NO:852; SEQ ID NO:447 and SEQ ID NO:852; SEQ ID NO:448 and SEQ ID NO:852; SEQ ID NO:449 and SEQ ID NO: 852; SEQ ID NO:450 and SEQ ID NO:852; SEQ ID NO:451 and SEQ ID NO:852; SEQ ID NO:452 and SEQ ID NO: 852; SEQ ID NO:453 and SEQ ID NO:852; SEQ ID NO: 454 and SEQ ID NO: 852; SEQ ID NO:455 and SEQ ID NO: 852; SEQ ID NO:456 and SEQ ID NO: 852; SEQ ID NO:457 and SEQ ID NO:852; SEQ ID NO:458 and SEQ ID NO: 852; SEQ ID NO:459 and SEQ ID NO:852; SEQ ID NO:460 and SEQ ID NO:852; SEQ ID NO:461 and SEQ ID NO:852; SEQ ID NO:462 and SEQ ID NO:852; SEQ ID NO: 463 and SEQ ID NO: 852; SEQ ID NO:464 and SEQ ID NO:852; SEQ ID NO:465 and SEQ ID NO:852; SEQ ID NO:466 and SEQ ID NO:852; SEQ ID NO:467 and SEQ ID NO: 852; SEQ ID NO:468 and SEQ ID NO: 852; SEQ ID NO:469 and SEQ ID NO:852; SEQ ID NO:470 and SEQ ID NO: 852; SEQ ID NO:471 and SEQ ID NO:852; SEQ ID NO: 472 and SEQ ID NO: 852; SEQ ID NO:473 and SEQ ID NO: 852; SEQ ID NO:474 and SEQ ID NO: 852; SEQ ID NO:475 and SEQ ID NO: 852; SEQ ID NO:476 and SEQ ID NO: 852; SEQ ID NO:477 and SEQ ID NO:852; SEQ ID NO:478 and SEQ ID NO:852; SEQ ID NO:479 and SEQ ID NO:852; SEQ ID NO:480 and SEQ ID NO:852; SEQ ID NO: 481 and SEQ ID NO: 852; SEQ ID NO:482 and SEQ ID NO: 852; SEQ ID NO:483 and SEQ ID NO:852; SEQ ID NO:484 and SEQ ID NO:852; SEQ ID NO:485 and SEQ ID NO: 852; SEQ ID NO:486 and SEQ ID NO:852; SEQ ID NO:487 and SEQ ID NO:852; SEQ ID NO:488 and SEQ ID NO:852; SEQ ID NO:489 and SEQ ID NO:852; SEQ ID NO: 490 and SEQ ID NO:852; SEQ ID NO:491 and SEQ ID NO:852; SEQ ID NO:492 and SEQ ID NO:852; SEQ ID NO:493 and SEQ ID NO:852; SEQ ID NO:494 and SEQ ID NO: 852; SEQ ID NO:495 and SEQ ID NO:852; SEQ ID NO:496 and SEQ ID NO:852; SEQ ID NO:497 and SEQ ID NO:852; SEQ ID NO:498 and SEQ ID NO:852; SEQ ID NO: 499 and SEQ ID NO: 852; SEQ ID NO:500 and SEQ ID NO:852; SEQ ID NO:501 and SEQ ID NO:852; SEQ ID NO:502 and SEQ ID NO:852; SEQ ID NO:503 and SEQ ID NO: 852; SEQ ID NO:504 and SEQ ID NO:852; SEQ ID NO:505 and SEQ ID NO:852; SEQ ID NO:506 and SEQ ID NO:852; SEQ ID NO:507 and SEQ ID NO:852; SEQ ID NO: 508 and SEQ ID NO:852; SEQ ID NO:509 and SEQ ID NO:852; SEQ ID NO:510 and SEQ ID NO:852; SEQ ID NO:511 and SEQ ID NO:852; SEQ ID NO:512 and SEQ ID NO: 852; SEQ ID NO:513 and SEQ ID NO:852; SEQ ID NO:514 and SEQ ID NO:852; SEQ ID NO:515 and SEQ ID NO:852; SEQ ID NO:516 and SEQ ID NO:852; SEQ ID NO: 517 and SEQ ID NO:852; SEQ ID NO:518 and SEQ ID NO:852; SEQ ID NO:519 and SEQ ID NO:852; SEQ ID NO:520 and SEQ ID NO:852; SEQ ID NO:521 and SEQ ID NO: 852; SEQ ID NO:522 and SEQ ID NO: 852; and SEQ ID NO:523 and SEQ ID NO: 852.

In some aspects, the CDR-H3-CDR-L3 pairs are selected from SEQ ID NO:357 and SEQ ID NO:853; SEQ ID NO:358 and SEQ ID NO:853; SEQ ID NO:359 and SEQ ID NO: 853; SEQ ID NO:360 and SEQ ID NO:853; SEQ ID NO:361 and SEQ ID NO:853; SEQ ID NO:362 and SEQ ID NO:853; SEQ ID NO:363 and SEQ ID NO:853; SEQ ID NO: 364 and SEQ ID NO:853; SEQ ID NO:365 and SEQ ID NO:853; SEQ ID NO:366 and SEQ ID NO:853; SEQ ID NO:367 and SEQ ID NO:853; SEQ ID NO:368 and SEQ ID NO: 853; SEQ ID NO:369 and SEQ ID NO:853; SEQ ID NO:370 and SEQ ID NO:853; SEQ ID NO:371 and SEQ ID NO:853; SEQ ID NO:372 and SEQ ID NO: 853; SEQ ID NO: 373 and SEQ ID NO: 853; SEQ ID NO:374 and SEQ ID NO:853; SEQ ID NO:375 and SEQ ID NO:853; SEQ ID NO:376 and SEQ ID NO:853; SEQ ID NO:377 and SEQ ID NO: 853; SEQ ID NO:378 and SEQ ID NO:853; SEQ ID NO:379 and SEQ ID NO:853; SEQ ID NO:380 and SEQ ID NO:853; SEQ ID NO:381 and SEQ ID NO:853; SEQ ID NO: 382 and SEQ ID NO: 853; SEQ ID NO:383 and SEQ ID NO:853; SEQ ID NO:384 and SEQ ID NO:853; SEQ ID NO:385 and SEQ ID NO:853; SEQ ID NO:386 and SEQ ID NO: 853; SEQ ID NO:387 and SEQ ID NO:853; SEQ ID NO:388 and SEQ ID NO:853; SEQ ID NO:389 and SEQ ID NO:853; SEQ ID NO:390 and SEQ ID NO:853; SEQ ID NO: 391 and SEQ ID NO:853; SEQ ID NO:392 and SEQ ID NO:853; SEQ ID NO:393 and SEQ ID NO:853; SEQ ID NO:394 and SEQ ID NO:853; SEQ ID NO:395 and SEQ ID NO: 853; SEQ ID NO:396 and SEQ ID NO: 853; SEQ ID NO:397 and SEQ ID NO:853; SEQ ID NO:398 and SEQ ID NO:853; SEQ ID NO:399 and SEQ ID NO:853; SEQ ID NO: 400 and SEQ ID NO: 853; SEQ ID NO:401 and SEQ ID NO:853; SEQ ID NO:402 and SEQ ID NO: 853; SEQ ID NO:403 and SEQ ID NO:853; SEQ ID NO:404 and SEQ ID NO: 853; SEQ ID NO:405 and SEQ ID NO: 853; SEQ ID NO:406 and SEQ ID NO:853; SEQ ID NO:407 and SEQ ID NO:853; SEQ ID NO:408 and SEQ ID NO:853; SEQ ID NO: 409 and SEQ ID NO: 853; SEQ ID NO:410 and SEQ ID NO: 853; SEQ ID NO:411 and SEQ ID NO:853; SEQ ID NO:412 and SEQ ID NO:853; SEQ ID NO:413 and SEQ ID NO: 853; SEQ ID NO:414 and SEQ ID NO:853; SEQ ID NO:415 and SEQ ID NO:853; SEQ ID NO:416 and SEQ ID NO: 853; SEQ ID NO:417 and SEQ ID NO:853; SEQ ID NO: 418 and SEQ ID NO: 853; SEQ ID NO:419 and SEQ ID NO:853; SEQ ID NO:420 and SEQ ID NO: 853; SEQ ID NO:421 and SEQ ID NO:853; SEQ ID NO:422 and SEQ ID NO: 853; SEQ ID NO:423 and SEQ ID NO:853; SEQ ID NO:424 and SEQ ID NO:853; SEQ ID NO:425 and SEQ ID NO:853; SEQ ID NO:426 and SEQ ID NO:853; SEQ ID NO: 427 and SEQ ID NO: 853; SEQ ID NO:428 and SEQ ID NO: 853; SEQ ID NO:429 and SEQ ID NO:853; SEQ ID NO:430 and SEQ ID NO:853; SEQ ID NO:431 and SEQ ID NO: 853; SEQ ID NO:432 and SEQ ID NO:853; SEQ ID NO:433 and SEQ ID NO:853; SEQ ID NO:434 and SEQ ID NO: 853; SEQ ID NO:435 and SEQ ID NO:853; SEQ ID NO: 436 and SEQ ID NO: 853; SEQ ID NO:437 and SEQ ID NO: 853; SEQ ID NO:438 and SEQ ID NO: 853; SEQ ID NO:439 and SEQ ID NO:853; SEQ ID NO:440 and SEQ ID NO: 853; SEQ ID NO:441 and SEQ ID NO:853; SEQ ID NO:442 and SEQ ID NO:853; SEQ ID NO:443 and SEQ ID NO:853; SEQ ID NO:444 and SEQ ID NO:853; SEQ ID NO: 445 and SEQ ID NO: 853; SEQ ID NO:446 and SEQ ID NO:853; SEQ ID NO:447 and SEQ ID NO: 853; SEQ ID NO:448 and SEQ ID NO:853; SEQ ID NO:449 and SEQ ID NO: 853; SEQ ID NO:450 and SEQ ID NO:853; SEQ ID NO:451 and SEQ ID NO:853; SEQ ID NO:452 and SEQ ID NO:853; SEQ ID NO:453 and SEQ ID NO:853; SEQ ID NO: 454 and SEQ ID NO: 853; SEQ ID NO:455 and SEQ ID NO:853; SEQ ID NO:456 and SEQ ID NO:853; SEQ ID NO:457 and SEQ ID NO:853; SEQ ID NO:458 and SEQ ID NO: 853; SEQ ID NO:459 and SEQ ID NO:853; SEQ ID NO:460 and SEQ ID NO:853; SEQ ID NO:461 and SEQ ID NO:853; SEQ ID NO:462 and SEQ ID NO:853; SEQ ID NO: 463 and SEQ ID NO:853; SEQ ID NO:464 and SEQ ID NO:853; SEQ ID NO:465 and SEQ ID NO:853; SEQ ID NO:466 and SEQ ID NO:853; SEQ ID NO:467 and SEQ ID NO: 853; SEQ ID NO:468 and SEQ ID NO:853; SEQ ID NO:469 and SEQ ID NO:853; SEQ ID NO:470 and SEQ ID NO:853; SEQ ID NO:471 and SEQ ID NO: 853; SEQ ID NO: 472 and SEQ ID NO: 853; SEQ ID NO:473 and SEQ ID NO:853; SEQ ID NO:474 and SEQ ID NO:853; SEQ ID NO:475 and SEQ ID NO:853; SEQ ID NO:476 and SEQ ID NO: 853; SEQ ID NO:477 and SEQ ID NO:853; SEQ ID NO:478 and SEQ ID NO:853; SEQ ID NO:479 and SEQ ID NO: 853; SEQ ID NO:480 and SEQ ID NO:853; SEQ ID NO: 481 and SEQ ID NO:853; SEQ ID NO:482 and SEQ ID NO:853; SEQ ID NO:483 and SEQ ID NO:853; SEQ ID NO:484 and SEQ ID NO:853; SEQ ID NO:485 and SEQ ID NO: 853; SEQ ID NO:486 and SEQ ID NO:853; SEQ ID NO:487 and SEQ ID NO:853; SEQ ID NO:488 and SEQ ID NO:853; SEQ ID NO:489 and SEQ ID NO:853; SEQ ID NO: 490 and SEQ ID NO: 853; SEQ ID NO:491 and SEQ ID NO: 853; SEQ ID NO:492 and SEQ ID NO: 853; SEQ ID NO:493 and SEQ ID NO: 853; SEQ ID NO:494 and SEQ ID NO: 853; SEQ ID NO:495 and SEQ ID NO: 853; SEQ ID NO:496 and SEQ ID NO:853; SEQ ID NO: 497 and SEQ ID NO:853; SEQ ID NO:498 and SEQ ID NO: 853; SEQ ID NO: 499 and SEQ ID NO: 853; SEQ ID NO:500 and SEQ ID NO: 853; SEQ ID NO:501 and SEQ ID NO:853; SEQ ID NO:502 and SEQ ID NO:853; SEQ ID NO:503 and SEQ ID NO: 853; SEQ ID NO:504 and SEQ ID NO:853; SEQ ID NO:505 and SEQ ID NO:853; SEQ ID NO:506 and SEQ ID NO:853; SEQ ID NO:507 and SEQ ID NO:853; SEQ ID NO: 508 and SEQ ID NO: 853; SEQ ID NO:509 and SEQ ID NO:853; SEQ ID NO:510 and SEQ ID NO:853; SEQ ID NO:511 and SEQ ID NO:853; SEQ ID NO:512 and SEQ ID NO: 853; SEQ ID NO:513 and SEQ ID NO:853; SEQ ID NO:514 and SEQ ID NO:853; SEQ ID NO:515 and SEQ ID NO:853; SEQ ID NO:516 and SEQ ID NO:853; SEQ ID NO: 517 and SEQ ID NO:853; SEQ ID NO:518 and SEQ ID NO:853; SEQ ID NO:519 and SEQ ID NO:853; SEQ ID NO:520 and SEQ ID NO:853; SEQ ID NO:521 and SEQ ID NO: 853; SEQ ID NO:522 and SEQ ID NO:853; and SEQ ID NO:523 and SEQ ID NO: 853.

### 5.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, 95%, 96%, 97%, 98%, or 99% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 5.7.2. CDR-H1-CDR-L1 Pairs

In some embodiments, the antibody comprises a CDR-H1 sequence and a CDR-L1 sequence. In some aspects, the CDR-H1 sequence is part of a V_{H} and the CDR-L1 sequence is part of a V_{L}.

In some aspects, the CDR-H1 sequence is a Chothia CDR-H1 sequence comprising, consisting of, or consisting essentially of any one of SEQ ID NOs: 4-170, and the CDR-L1 sequence is a CDR-L1 sequence comprising, consisting of, or consisting essentially of any one of SEQ ID NOs: 839-843.

In some aspects, the CDR-H1 sequence is a Kabat CDR-H1 sequence comprising, consisting of, or consisting essentially of any one of SEQ ID NOs: 171-337, and the CDR-L1 sequence is a CDR-L1 sequence comprising, consisting of, or consisting essentially of any one of SEQ ID NOs: 839-843.

### 5.7.2.1. Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, 95%, 96%, 97%, 98%, or 99% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, 95%, 96%, 97%, 98%, or 99% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

### 5.7.3. CDR-H2-CDR-L2 Pairs

In some embodiments, the antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of any one of SEQ ID NOs: 338-504, and the CDR-L2 sequence is a CDR-L2 sequence comprising, consisting of, or consisting essentially of any one of SEQ ID NOs: 844-848.

In some aspects, the CDR-H1 sequence is a Kabat CDR-H2 sequence comprising, consisting of, or consisting essentially of any one of SEQ ID NOs: 505-671, and the CDR-L2 sequence is a CDR-L2 sequence comprising, consisting of, or consisting essentially of any one of SEQ ID NOs: 844-848.

### 5.7.3.1. Variants of CDR-H2-CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, 95%, 96%, 97%, 98%, or 99% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, 95%, 96%, 97%, 98%, or 99% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

5.7.4. V_{H}-V_{L} Pairs

In some embodiments, the antibody comprises a V_{H} sequence and a V_{L} sequence.

In some aspects, the V_{H} sequence is a V_{H} sequence comprising, consisting of, or consisting essentially of any one of SEQ ID NOs: 854-1020, and the V_{L} sequence is a V_{L} sequence comprising, consisting of, or consisting essentially of any one of SEQ ID NOs: 1021-1026.

In some aspects, the V_{H}-V_{L} pairs are selected from SEQ ID NO:854 and SEQ ID NO: 1021; SEQ ID NO:855 and SEQ ID NO:1021; SEQ ID NO:856 and SEQ ID NO:1021; SEQ ID NO:857 and SEQ ID NO:1021; SEQ ID NO:858 and SEQ ID NO:1021; SEQ ID NO: 859 and SEQ ID NO: 1021; SEQ ID NO: 860 and SEQ ID NO:1021; SEQ ID NO:861 and SEQ ID NO:1021; SEQ ID NO:862 and SEQ ID NO: 1021; SEQ ID NO:863 and SEQ ID NO: 1021; SEQ ID NO:864 and SEQ ID NO:1021; SEQ ID NO:865 and SEQ ID NO: 1021; SEQ ID NO:866 and SEQ ID NO:1021; SEQ ID NO:867 and SEQ ID NO:1021; SEQ ID NO:868 and SEQ ID NO:1021; SEQ ID NO:869 and SEQ ID NO:1021; SEQ ID NO: 870 and SEQ ID NO: 1021; SEQ ID NO:871 and SEQ ID NO:1021; SEQ ID NO:872 and SEQ ID NO: 1021; SEQ ID NO:873 and SEQ ID NO:1021; SEQ ID NO:874 and SEQ ID NO: 1021; SEQ ID NO:875 and SEQ ID NO: 1021; SEQ ID NO:876 and SEQ ID NO: 1021; SEQ ID NO:877 and SEQ ID NO:1021; SEQ ID NO:878 and SEQ ID NO:1021; SEQ ID NO:879 and SEQ ID NO:1021; SEQ ID NO:880 and SEQ ID NO: 1021; SEQ ID NO: 881 and SEQ ID NO: 1021; SEQ ID NO:882 and SEQ ID NO: 1021; SEQ ID NO:883 and SEQ ID NO: 1021; SEQ ID NO:884 and SEQ ID NO:1021; SEQ ID NO:885 and SEQ ID NO: 1021; SEQ ID NO:886 and SEQ ID NO:1021; SEQ ID NO:887 and SEQ ID NO: 1021; SEQ ID NO:888 and SEQ ID NO:1021; SEQ ID NO:889 and SEQ ID NO:1021; SEQ ID NO:890 and SEQ ID NO: 1021; SEQ ID NO:891 and SEQ ID NO: 1021; SEQ ID NO: 892 and SEQ ID NO: 1021; SEQ ID NO:893 and SEQ ID NO: 1021; SEQ ID NO: 894 and SEQ ID NO:1021; SEQ ID NO:895 and SEQ ID NO:1021; SEQ ID NO:896 and SEQ ID NO: 1021; SEQ ID NO: 897 and SEQ ID NO: 1021; SEQ ID NO:898 and SEQ ID NO: 1021; SEQ ID NO:899 and SEQ ID NO:1021; SEQ ID NO:900 and SEQ ID NO:1021; SEQ ID NO:901 and SEQ ID NO:1021; SEQ ID NO:902 and SEQ ID NO:1021; SEQ ID NO: 903 and SEQ ID NO: 1021; SEQ ID NO:904 and SEQ ID NO: 1021; SEQ ID NO:905 and SEQ ID NO: 1021; SEQ ID NO:906 and SEQ ID NO: 1021; SEQ ID NO:907 and SEQ ID NO: 1021; SEQ ID NO:908 and SEQ ID NO: 1021; SEQ ID NO:909 and SEQ ID NO: 1021; SEQ ID NO:910 and SEQ ID NO:1021; SEQ ID NO:911 and SEQ ID NO:1021; SEQ ID NO:912 and SEQ ID NO:1021; SEQ ID NO:913 and SEQ ID NO: 1021; SEQ ID NO: 914 and SEQ ID NO: 1021; SEQ ID NO:915 and SEQ ID NO:1021; SEQ ID NO:916 and SEQ ID NO: 1021; SEQ ID NO:917 and SEQ ID NO:1021; SEQ ID NO:918 and SEQ ID NO: 1021; SEQ ID NO:919 and SEQ ID NO: 1021; SEQ ID NO:920 and SEQ ID NO: 1021; SEQ ID NO:921 and SEQ ID NO:1021; SEQ ID NO:922 and SEQ ID NO:1021; SEQ ID NO:923 and SEQ ID NO:1021; SEQ ID NO:924 and SEQ ID NO:1021; SEQ ID NO: 925 and SEQ ID NO: 1021; SEQ ID NO:926 and SEQ ID NO: 1021; SEQ ID NO:927 and SEQ ID NO:1021; SEQ ID NO:928 and SEQ ID NO:1021; SEQ ID NO:929 and SEQ ID NO: 1021; SEQ ID NO:930 and SEQ ID NO:1021; SEQ ID NO:931 and SEQ ID NO: 1021; SEQ ID NO:932 and SEQ ID NO: 1021; SEQ ID NO:933 and SEQ ID NO:1021; SEQ ID NO:934 and SEQ ID NO:1021; SEQ ID NO:935 and SEQ ID NO: 1021; SEQ ID NO: 936 and SEQ ID NO: 1021; SEQ ID NO:937 and SEQ ID NO: 1021; SEQ ID NO:938 and SEQ ID NO: 1021; SEQ ID NO:939 and SEQ ID NO: 1021; SEQ ID NO:940 and SEQ ID NO: 1021; SEQ ID NO:941 and SEQ ID NO: 1021; SEQ ID NO:942 and SEQ ID NO: 1021; SEQ ID NO:943 and SEQ ID NO:1021; SEQ ID NO:944 and SEQ ID NO: 1021; SEQ ID NO:945 and SEQ ID NO: 1021; SEQ ID NO:946 and SEQ ID NO: 1021; SEQ ID NO: 947 and SEQ ID NO: 1021; SEQ ID NO:948 and SEQ ID NO: 1021; SEQ ID NO:949 and SEQ ID NO: 1021; SEQ ID NO:950 and SEQ ID NO: 1021; SEQ ID NO:951 and SEQ ID NO: 1021; SEQ ID NO:952 and SEQ ID NO: 1021; SEQ ID NO:953 and SEQ ID NO: 1021; SEQ ID NO:954 and SEQ ID NO: 1021; SEQ ID NO:955 and SEQ ID NO:1021; SEQ ID NO:956 and SEQ ID NO:1021; SEQ ID NO:957 and SEQ ID NO:1021; SEQ ID NO: 958 and SEQ ID NO: 1021; SEQ ID NO:959 and SEQ ID NO:1021; SEQ ID NO:960 and SEQ ID NO: 1021; SEQ ID NO:961 and SEQ ID NO:1021; SEQ ID NO:962 and SEQ ID NO: 1021; SEQ ID NO:963 and SEQ ID NO:1021; SEQ ID NO:964 and SEQ ID NO: 1021; SEQ ID NO:965 and SEQ ID NO: 1021; SEQ ID NO:966 and SEQ ID NO:1021; SEQ ID NO:967 and SEQ ID NO:1021; SEQ ID NO:968 and SEQ ID NO:1021; SEQ ID NO: 969 and SEQ ID NO: 1021; SEQ ID NO:970 and SEQ ID NO:1021; SEQ ID NO:971 and SEQ ID NO: 1021; SEQ ID NO:972 and SEQ ID NO: 1021; SEQ ID NO:973 and SEQ ID NO: 1021; SEQ ID NO:974 and SEQ ID NO:1021; SEQ ID NO:975 and SEQ ID NO: 1021; SEQ ID NO:976 and SEQ ID NO:1021; SEQ ID NO:977 and SEQ ID NO:1021; SEQ ID NO:978 and SEQ ID NO:1021; SEQ ID NO:979 and SEQ ID NO:1021; SEQ ID NO: 980 and SEQ ID NO: 1021; SEQ ID NO:981 and SEQ ID NO: 1021; SEQ ID NO:982 and SEQ ID NO:1021; SEQ ID NO:983 and SEQ ID NO:1021; SEQ ID NO:984 and SEQ ID NO: 1021; SEQ ID NO:985 and SEQ ID NO:1021; SEQ ID NO:986 and SEQ ID NO: 1021; SEQ ID NO:987 and SEQ ID NO:1021; SEQ ID NO:988 and SEQ ID NO:1021; SEQ ID NO:989 and SEQ ID NO: 1021; SEQ ID NO:990 and SEQ ID NO: 1021; SEQ ID NO: 991 and SEQ ID NO: 1021; SEQ ID NO:992 and SEQ ID NO: 1021; SEQ ID NO:993 and SEQ ID NO:1021; SEQ ID NO:994 and SEQ ID NO:1021; SEQ ID NO:995 and SEQ ID NO: 1021; SEQ ID NO:996 and SEQ ID NO: 1021; SEQ ID NO:997 and SEQ ID NO: 1021; SEQ ID NO:998 and SEQ ID NO: 1021; SEQ ID NO:999 and SEQ ID NO:1021; SEQ ID NO:1000 and SEQ ID NO:1021; SEQ ID NO:1001 and SEQ ID NO:1021; SEQ ID NO: 1002 and SEQ ID NO:1021; SEQ ID NO:1003 and SEQ ID NO:1021; SEQ ID NO: 1004 and SEQ ID NO:1021; SEQ ID NO: 1005 and SEQ ID NO:1021; SEQ ID NO: 1006 and SEQ ID NO:1021; SEQ ID NO: 1007 and SEQ ID NO: 1021; SEQ ID NO: 1008 and SEQ ID NO:1021; SEQ ID NO:1009 and SEQ ID NO:1021; SEQ ID NO: 1010 and SEQ ID NO: 1021; SEQ ID NO:1011 and SEQ ID NO: 1021; SEQ ID NO: 1012 and SEQ ID NO:1021; SEQ ID NO:1013 and SEQ ID NO: 1021; SEQ ID NO: 1014 and SEQ ID NO:1021; SEQ ID NO:1015 and SEQ ID NO:1021; SEQ ID NO: 1016 and SEQ ID NO:1021; SEQ ID NO: 1017 and SEQ ID NO: 1021; SEQ ID NO: 1018 and SEQ ID NO:1021; SEQ ID NO: 1019 and SEQ ID NO:1021; and SEQ ID NO: 1020 and SEQ ID NO:1021.

In some aspects, the V_{H}-V_{L} pairs are selected from SEQ ID NO:854 and SEQ ID NO: 1022; SEQ ID NO:855 and SEQ ID NO:1022; SEQ ID NO:856 and SEQ ID NO:1022; SEQ ID NO:857 and SEQ ID NO: 1022; SEQ ID NO:858 and SEQ ID NO: 1022; SEQ ID NO: 859 and SEQ ID NO: 1022; SEQ ID NO:860 and SEQ ID NO: 1022; SEQ ID NO:861 and SEQ ID NO:1022; SEQ ID NO:862 and SEQ ID NO:1022; SEQ ID NO:863 and SEQ ID NO: 1022; SEQ ID NO:864 and SEQ ID NO:1022; SEQ ID NO:865 and SEQ ID NO: 1022; SEQ ID NO:866 and SEQ ID NO:1022; SEQ ID NO:867 and SEQ ID NO:1022; SEQ ID NO:868 and SEQ ID NO: 1022; SEQ ID NO:869 and SEQ ID NO:1022; SEQ ID NO: 870 and SEQ ID NO: 1022; SEQ ID NO:871 and SEQ ID NO: 1022; SEQ ID NO:872 and SEQ ID NO:1022; SEQ ID NO:873 and SEQ ID NO:1022; SEQ ID NO:874 and SEQ ID NO: 1022; SEQ ID NO:875 and SEQ ID NO:1022; SEQ ID NO:876 and SEQ ID NO: 1022; SEQ ID NO:877 and SEQ ID NO:1022; SEQ ID NO:878 and SEQ ID NO:1022; SEQ ID NO:879 and SEQ ID NO:1022; SEQ ID NO:880 and SEQ ID NO: 1022; SEQ ID NO: 881 and SEQ ID NO:1022; SEQ ID NO:882 and SEQ ID NO:1022; SEQ ID NO:883 and SEQ ID NO:1022; SEQ ID NO:884 and SEQ ID NO:1022; SEQ ID NO:885 and SEQ ID NO: 1022; SEQ ID NO:886 and SEQ ID NO: 1022; SEQ ID NO:887 and SEQ ID NO: 1022; SEQ ID NO:888 and SEQ ID NO:1022; SEQ ID NO:889 and SEQ ID NO:1022; SEQ ID NO:890 and SEQ ID NO: 1022; SEQ ID NO:891 and SEQ ID NO:1022; SEQ ID NO: 892 and SEQ ID NO: 1022; SEQ ID NO:893 and SEQ ID NO: 1022; SEQ ID NO:894 and SEQ ID NO: 1022; SEQ ID NO:895 and SEQ ID NO: 1022; SEQ ID NO:896 and SEQ ID NO: 1022; SEQ ID NO:897 and SEQ ID NO:1022; SEQ ID NO:898 and SEQ ID NO: 1022; SEQ ID NO:899 and SEQ ID NO: 1022; SEQ ID NO:900 and SEQ ID NO:1022; SEQ ID NO:901 and SEQ ID NO:1022; SEQ ID NO:902 and SEQ ID NO: 1022; SEQ ID NO: 903 and SEQ ID NO: 1022; SEQ ID NO:904 and SEQ ID NO: 1022; SEQ ID NO:905 and SEQ ID NO: 1022; SEQ ID NO:906 and SEQ ID NO: 1022; SEQ ID NO:907 and SEQ ID NO: 1022; SEQ ID NO:908 and SEQ ID NO: 1022; SEQ ID NO:909 and SEQ ID NO: 1022; SEQ ID NO:910 and SEQ ID NO: 1022; SEQ ID NO:911 and SEQ ID NO: 1022; SEQ ID NO:912 and SEQ ID NO:1022; SEQ ID NO:913 and SEQ ID NO:1022; SEQ ID NO: 914 and SEQ ID NO: 1022; SEQ ID NO:915 and SEQ ID NO:1022; SEQ ID NO:916 and SEQ ID NO: 1022; SEQ ID NO:917 and SEQ ID NO: 1022; SEQ ID NO:918 and SEQ ID NO: 1022; SEQ ID NO:919 and SEQ ID NO: 1022; SEQ ID NO:920 and SEQ ID NO: 1022; SEQ ID NO:921 and SEQ ID NO:1022; SEQ ID NO:922 and SEQ ID NO:1022; SEQ ID NO:923 and SEQ ID NO:1022; SEQ ID NO:924 and SEQ ID NO:1022; SEQ ID NO: 925 and SEQ ID NO: 1022; SEQ ID NO:926 and SEQ ID NO: 1022; SEQ ID NO:927 and SEQ ID NO:1022; SEQ ID NO:928 and SEQ ID NO: 1022; SEQ ID NO:929 and SEQ ID NO: 1022; SEQ ID NO:930 and SEQ ID NO:1022; SEQ ID NO:931 and SEQ ID NO: 1022; SEQ ID NO:932 and SEQ ID NO:1022; SEQ ID NO:933 and SEQ ID NO:1022; SEQ ID NO:934 and SEQ ID NO:1022; SEQ ID NO:935 and SEQ ID NO: 1022; SEQ ID NO: 936 and SEQ ID NO: 1022; SEQ ID NO:937 and SEQ ID NO: 1022; SEQ ID NO:938 and SEQ ID NO: 1022; SEQ ID NO:939 and SEQ ID NO: 1022; SEQ ID NO:940 and SEQ ID NO: 1022; SEQ ID NO:941 and SEQ ID NO: 1022; SEQ ID NO:942 and SEQ ID NO: 1022; SEQ ID NO:943 and SEQ ID NO:1022; SEQ ID NO:944 and SEQ ID NO:1022; SEQ ID NO:945 and SEQ ID NO: 1022; SEQ ID NO:946 and SEQ ID NO: 1022; SEQ ID NO: 947 and SEQ ID NO: 1022; SEQ ID NO:948 and SEQ ID NO: 1022; SEQ ID NO:949 and SEQ ID NO:1022; SEQ ID NO:950 and SEQ ID NO:1022; SEQ ID NO:951 and SEQ ID NO: 1022; SEQ ID NO:952 and SEQ ID NO:1022; SEQ ID NO:953 and SEQ ID NO: 1022; SEQ ID NO:954 and SEQ ID NO:1022; SEQ ID NO:955 and SEQ ID NO:1022; SEQ ID NO:956 and SEQ ID NO:1022; SEQ ID NO:957 and SEQ ID NO: 1022; SEQ ID NO: 958 and SEQ ID NO: 1022; SEQ ID NO:959 and SEQ ID NO: 1022; SEQ ID NO:960 and SEQ ID NO: 1022; SEQ ID NO:961 and SEQ ID NO: 1022; SEQ ID NO:962 and SEQ ID NO: 1022; SEQ ID NO:963 and SEQ ID NO: 1022; SEQ ID NO:964 and SEQ ID NO: 1022; SEQ ID NO:965 and SEQ ID NO: 1022; SEQ ID NO:966 and SEQ ID NO: 1022; SEQ ID NO:967 and SEQ ID NO:1022; SEQ ID NO:968 and SEQ ID NO:1022; SEQ ID NO: 969 and SEQ ID NO: 1022; SEQ ID NO:970 and SEQ ID NO: 1022; SEQ ID NO:971 and SEQ ID NO: 1022; SEQ ID NO:972 and SEQ ID NO:1022; SEQ ID NO:973 and SEQ ID NO: 1022; SEQ ID NO:974 and SEQ ID NO:1022; SEQ ID NO:975 and SEQ ID NO: 1022; SEQ ID NO:976 and SEQ ID NO:1022; SEQ ID NO:977 and SEQ ID NO:1022; SEQ ID NO:978 and SEQ ID NO: 1022; SEQ ID NO:979 and SEQ ID NO: 1022; SEQ ID NO: 980 and SEQ ID NO: 1022; SEQ ID NO:981 and SEQ ID NO:1022; SEQ ID NO:982 and SEQ ID NO: 1022; SEQ ID NO:983 and SEQ ID NO: 1022; SEQ ID NO:984 and SEQ ID NO: 1022; SEQ ID NO:985 and SEQ ID NO: 1022; SEQ ID NO:986 and SEQ ID NO: 1022; SEQ ID NO:987 and SEQ ID NO:1022; SEQ ID NO:988 and SEQ ID NO:1022; SEQ ID NO:989 and SEQ ID NO: 1022; SEQ ID NO:990 and SEQ ID NO: 1022; SEQ ID NO: 991 and SEQ ID NO: 1022; SEQ ID NO:992 and SEQ ID NO:1022; SEQ ID NO:993 and SEQ ID NO: 1022; SEQ ID NO: 994 and SEQ ID NO: 1022; SEQ ID NO:995 and SEQ ID NO: 1022; SEQ ID NO:996 and SEQ ID NO: 1022; SEQ ID NO:997 and SEQ ID NO: 1022; SEQ ID NO:998 and SEQ ID NO: 1022; SEQ ID NO:999 and SEQ ID NO: 1022; SEQ ID NO:1000 and SEQ ID NO:1022; SEQ ID NO:1001 and SEQ ID NO:1022; SEQ ID NO: 1002 and SEQ ID NO:1022; SEQ ID NO:1003 and SEQ ID NO:1022; SEQ ID NO: 1004 and SEQ ID NO: 1022; SEQ ID NO: 1005 and SEQ ID NO: 1022; SEQ ID NO: 1006 and SEQ ID NO: 1022; SEQ ID NO: 1007 and SEQ ID NO: 1022; SEQ ID NO: 1008 and SEQ ID NO: 1022; SEQ ID NO: 1009 and SEQ ID NO: 1022; SEQ ID NO: 1010 and SEQ ID NO:1022; SEQ ID NO:1011 and SEQ ID NO:1022; SEQ ID NO: 1012 and SEQ ID NO:1022; SEQ ID NO:1013 and SEQ ID NO:1022; SEQ ID NO: 1014 and SEQ ID NO:1022; SEQ ID NO:1015 and SEQ ID NO:1022; SEQ ID NO: 1016 and SEQ ID NO:1022; SEQ ID NO:1017 and SEQ ID NO: 1022; SEQ ID NO: 1018 and SEQ ID NO:1022; SEQ ID NO:1019 and SEQ ID NO: 1022; and SEQ ID NO: 1020 and SEQ ID NO:1022.

In some aspects, the V_{H}-V_{L} pairs are selected from SEQ ID NO:854 and SEQ ID NO: 1023; SEQ ID NO:855 and SEQ ID NO:1023; SEQ ID NO:856 and SEQ ID NO:1023; SEQ ID NO:857 and SEQ ID NO:1023; SEQ ID NO:858 and SEQ ID NO: 1023; SEQ ID NO: 859 and SEQ ID NO: 1023; SEQ ID NO: 860 and SEQ ID NO: 1023; SEQ ID NO:861 and SEQ ID NO: 1023; SEQ ID NO:862 and SEQ ID NO:1023; SEQ ID NO:863 and SEQ ID NO: 1023; SEQ ID NO:864 and SEQ ID NO: 1023; SEQ ID NO:865 and SEQ ID NO: 1023; SEQ ID NO:866 and SEQ ID NO:1023; SEQ ID NO:867 and SEQ ID NO:1023; SEQ ID NO:868 and SEQ ID NO: 1023; SEQ ID NO:869 and SEQ ID NO:1023; SEQ ID NO: 870 and SEQ ID NO: 1023; SEQ ID NO:871 and SEQ ID NO: 1023; SEQ ID NO:872 and SEQ ID NO: 1023; SEQ ID NO:873 and SEQ ID NO:1023; SEQ ID NO:874 and SEQ ID NO: 1023; SEQ ID NO:875 and SEQ ID NO: 1023; SEQ ID NO:876 and SEQ ID NO: 1023; SEQ ID NO:877 and SEQ ID NO:1023; SEQ ID NO:878 and SEQ ID NO:1023; SEQ ID NO:879 and SEQ ID NO:1023; SEQ ID NO:880 and SEQ ID NO:1023; SEQ ID NO: 881 and SEQ ID NO: 1023; SEQ ID NO:882 and SEQ ID NO:1023; SEQ ID NO:883 and SEQ ID NO: 1023; SEQ ID NO:884 and SEQ ID NO:1023; SEQ ID NO:885 and SEQ ID NO: 1023; SEQ ID NO:886 and SEQ ID NO: 1023; SEQ ID NO:887 and SEQ ID NO: 1023; SEQ ID NO:888 and SEQ ID NO:1023; SEQ ID NO:889 and SEQ ID NO:1023; SEQ ID NO: 890 and SEQ ID NO: 1023; SEQ ID NO:891 and SEQ ID NO: 1023; SEQ ID NO: 892 and SEQ ID NO: 1023; SEQ ID NO:893 and SEQ ID NO: 1023; SEQ ID NO: 894 and SEQ ID NO: 1023; SEQ ID NO:895 and SEQ ID NO: 1023; SEQ ID NO:896 and SEQ ID NO: 1023; SEQ ID NO:897 and SEQ ID NO:1023; SEQ ID NO:898 and SEQ ID NO: 1023; SEQ ID NO: 899 and SEQ ID NO: 1023; SEQ ID NO:900 and SEQ ID NO:1023; SEQ ID NO:901 and SEQ ID NO:1023; SEQ ID NO:902 and SEQ ID NO:1023; SEQ ID NO: 903 and SEQ ID NO: 1023; SEQ ID NO:904 and SEQ ID NO: 1023; SEQ ID NO:905 and SEQ ID NO: 1023; SEQ ID NO:906 and SEQ ID NO:1023; SEQ ID NO:907 and SEQ ID NO: 1023; SEQ ID NO:908 and SEQ ID NO: 1023; SEQ ID NO:909 and SEQ ID NO: 1023; SEQ ID NO:910 and SEQ ID NO: 1023; SEQ ID NO:911 and SEQ ID NO:1023; SEQ ID NO:912 and SEQ ID NO: 1023; SEQ ID NO:913 and SEQ ID NO: 1023; SEQ ID NO: 914 and SEQ ID NO: 1023; SEQ ID NO:915 and SEQ ID NO: 1023; SEQ ID NO:916 and SEQ ID NO: 1023; SEQ ID NO:917 and SEQ ID NO: 1023; SEQ ID NO:918 and SEQ ID NO: 1023; SEQ ID NO:919 and SEQ ID NO: 1023; SEQ ID NO:920 and SEQ ID NO: 1023; SEQ ID NO:921 and SEQ ID NO: 1023; SEQ ID NO:922 and SEQ ID NO:1023; SEQ ID NO:923 and SEQ ID NO: 1023; SEQ ID NO:924 and SEQ ID NO: 1023; SEQ ID NO: 925 and SEQ ID NO: 1023; SEQ ID NO:926 and SEQ ID NO: 1023; SEQ ID NO:927 and SEQ ID NO: 1023; SEQ ID NO:928 and SEQ ID NO: 1023; SEQ ID NO:929 and SEQ ID NO: 1023; SEQ ID NO:930 and SEQ ID NO: 1023; SEQ ID NO:931 and SEQ ID NO: 1023; SEQ ID NO:932 and SEQ ID NO: 1023; SEQ ID NO:933 and SEQ ID NO: 1023; SEQ ID NO:934 and SEQ ID NO:1023; SEQ ID NO:935 and SEQ ID NO:1023; SEQ ID NO: 936 and SEQ ID NO: 1023; SEQ ID NO:937 and SEQ ID NO: 1023; SEQ ID NO:938 and SEQ ID NO: 1023; SEQ ID NO:939 and SEQ ID NO: 1023; SEQ ID NO:940 and SEQ ID NO: 1023; SEQ ID NO:941 and SEQ ID NO:1023; SEQ ID NO:942 and SEQ ID NO: 1023; SEQ ID NO:943 and SEQ ID NO: 1023; SEQ ID NO:944 and SEQ ID NO: 1023; SEQ ID NO:945 and SEQ ID NO: 1023; SEQ ID NO:946 and SEQ ID NO: 1023; SEQ ID NO: 947 and SEQ ID NO: 1023; SEQ ID NO:948 and SEQ ID NO: 1023; SEQ ID NO:949 and SEQ ID NO:1023; SEQ ID NO:950 and SEQ ID NO: 1023; SEQ ID NO:951 and SEQ ID NO: 1023; SEQ ID NO:952 and SEQ ID NO: 1023; SEQ ID NO:953 and SEQ ID NO: 1023; SEQ ID NO:954 and SEQ ID NO: 1023; SEQ ID NO:955 and SEQ ID NO:1023; SEQ ID NO:956 and SEQ ID NO:1023; SEQ ID NO:957 and SEQ ID NO: 1023; SEQ ID NO: 958 and SEQ ID NO: 1023; SEQ ID NO:959 and SEQ ID NO:1023; SEQ ID NO:960 and SEQ ID NO: 1023; SEQ ID NO:961 and SEQ ID NO: 1023; SEQ ID NO:962 and SEQ ID NO: 1023; SEQ ID NO:963 and SEQ ID NO: 1023; SEQ ID NO:964 and SEQ ID NO: 1023; SEQ ID NO:965 and SEQ ID NO: 1023; SEQ ID NO:966 and SEQ ID NO:1023; SEQ ID NO:967 and SEQ ID NO: 1023; SEQ ID NO:968 and SEQ ID NO:1023; SEQ ID NO: 969 and SEQ ID NO: 1023; SEQ ID NO:970 and SEQ ID NO: 1023; SEQ ID NO:971 and SEQ ID NO:1023; SEQ ID NO:972 and SEQ ID NO: 1023; SEQ ID NO:973 and SEQ ID NO: 1023; SEQ ID NO:974 and SEQ ID NO: 1023; SEQ ID NO:975 and SEQ ID NO: 1023; SEQ ID NO:976 and SEQ ID NO:1023; SEQ ID NO:977 and SEQ ID NO:1023; SEQ ID NO:978 and SEQ ID NO: 1023; SEQ ID NO:979 and SEQ ID NO: 1023; SEQ ID NO: 980 and SEQ ID NO: 1023; SEQ ID NO:981 and SEQ ID NO:1023; SEQ ID NO:982 and SEQ ID NO: 1023; SEQ ID NO:983 and SEQ ID NO: 1023; SEQ ID NO:984 and SEQ ID NO: 1023; SEQ ID NO:985 and SEQ ID NO: 1023; SEQ ID NO:986 and SEQ ID NO: 1023; SEQ ID NO:987 and SEQ ID NO:1023; SEQ ID NO:988 and SEQ ID NO:1023; SEQ ID NO:989 and SEQ ID NO: 1023; SEQ ID NO:990 and SEQ ID NO: 1023; SEQ ID NO: 991 and SEQ ID NO: 1023; SEQ ID NO:992 and SEQ ID NO: 1023; SEQ ID NO:993 and SEQ ID NO: 1023; SEQ ID NO:994 and SEQ ID NO: 1023; SEQ ID NO:995 and SEQ ID NO: 1023; SEQ ID NO:996 and SEQ ID NO: 1023; SEQ ID NO:997 and SEQ ID NO: 1023; SEQ ID NO:998 and SEQ ID NO: 1023; SEQ ID NO:999 and SEQ ID NO: 1023; SEQ ID NO:1000 and SEQ ID NO:1023; SEQ ID NO:1001 and SEQ ID NO:1023; SEQ ID NO: 1002 and SEQ ID NO:1023; SEQ ID NO:1003 and SEQ ID NO: 1023; SEQ ID NO: 1004 and SEQ ID NO: 1023; SEQ ID NO: 1005 and SEQ ID NO: 1023; SEQ ID NO: 1006 and SEQ ID NO:1023; SEQ ID NO: 1007 and SEQ ID NO: 1023; SEQ ID NO: 1008 and SEQ ID NO: 1023; SEQ ID NO: 1009 and SEQ ID NO: 1023; SEQ ID NO: 1010 and SEQ ID NO: 1023; SEQ ID NO: 1011 and SEQ ID NO: 1023; SEQ ID NO: 1012 and SEQ ID NO:1023; SEQ ID NO:1013 and SEQ ID NO:1023; SEQ ID NO: 1014 and SEQ ID NO:1023; SEQ ID NO:1015 and SEQ ID NO: 1023; SEQ ID NO: 1016 and SEQ ID NO: 1023; SEQ ID NO:1017 and SEQ ID NO: 1023; SEQ ID NO: 1018 and SEQ ID NO:1023; SEQ ID NO: 1019 and SEQ ID NO: 1023; and SEQ ID NO: 1020 and SEQ ID NO:1023.

In some aspects, the V_{H}-V_{L} pairs are selected from SEQ ID NO:854 and SEQ ID NO: 1024; SEQ ID NO:855 and SEQ ID NO:1024; SEQ ID NO:856 and SEQ ID NO:1024; SEQ ID NO:857 and SEQ ID NO:1024; SEQ ID NO:858 and SEQ ID NO:1024; SEQ ID NO: 859 and SEQ ID NO: 1024; SEQ ID NO:860 and SEQ ID NO: 1024; SEQ ID NO:861 and SEQ ID NO:1024; SEQ ID NO:862 and SEQ ID NO:1024; SEQ ID NO:863 and SEQ ID NO: 1024; SEQ ID NO:864 and SEQ ID NO: 1024; SEQ ID NO:865 and SEQ ID NO: 1024; SEQ ID NO:866 and SEQ ID NO:1024; SEQ ID NO:867 and SEQ ID NO:1024; SEQ ID NO:868 and SEQ ID NO:1024; SEQ ID NO:869 and SEQ ID NO:1024; SEQ ID NO: 870 and SEQ ID NO: 1024; SEQ ID NO:871 and SEQ ID NO: 1024; SEQ ID NO:872 and SEQ ID NO: 1024; SEQ ID NO:873 and SEQ ID NO: 1024; SEQ ID NO:874 and SEQ ID NO: 1024; SEQ ID NO:875 and SEQ ID NO:1024; SEQ ID NO:876 and SEQ ID NO: 1024; SEQ ID NO:877 and SEQ ID NO:1024; SEQ ID NO:878 and SEQ ID NO:1024; SEQ ID NO:879 and SEQ ID NO: 1024; SEQ ID NO:880 and SEQ ID NO:1024; SEQ ID NO: 881 and SEQ ID NO:1024; SEQ ID NO:882 and SEQ ID NO:1024; SEQ ID NO:883 and SEQ ID NO:1024; SEQ ID NO: 884 and SEQ ID NO: 1024; SEQ ID NO:885 and SEQ ID NO: 1024; SEQ ID NO:886 and SEQ ID NO: 1024; SEQ ID NO:887 and SEQ ID NO: 1024; SEQ ID NO: 888 and SEQ ID NO: 1024; SEQ ID NO:889 and SEQ ID NO: 1024; SEQ ID NO:890 and SEQ ID NO: 1024; SEQ ID NO:891 and SEQ ID NO: 1024; SEQ ID NO: 892 and SEQ ID NO: 1024; SEQ ID NO:893 and SEQ ID NO: 1024; SEQ ID NO:894 and SEQ ID NO: 1024; SEQ ID NO:895 and SEQ ID NO:1024; SEQ ID NO:896 and SEQ ID NO: 1024; SEQ ID NO:897 and SEQ ID NO: 1024; SEQ ID NO:898 and SEQ ID NO: 1024; SEQ ID NO:899 and SEQ ID NO:1024; SEQ ID NO:900 and SEQ ID NO:1024; SEQ ID NO:901 and SEQ ID NO:1024; SEQ ID NO:902 and SEQ ID NO: 1024; SEQ ID NO: 903 and SEQ ID NO: 1024; SEQ ID NO:904 and SEQ ID NO: 1024; SEQ ID NO:905 and SEQ ID NO: 1024; SEQ ID NO:906 and SEQ ID NO: 1024; SEQ ID NO:907 and SEQ ID NO: 1024; SEQ ID NO:908 and SEQ ID NO:1024; SEQ ID NO:909 and SEQ ID NO: 1024; SEQ ID NO:910 and SEQ ID NO: 1024; SEQ ID NO:911 and SEQ ID NO:1024; SEQ ID NO:912 and SEQ ID NO: 1024; SEQ ID NO:913 and SEQ ID NO: 1024; SEQ ID NO: 914 and SEQ ID NO: 1024; SEQ ID NO:915 and SEQ ID NO: 1024; SEQ ID NO:916 and SEQ ID NO:1024; SEQ ID NO:917 and SEQ ID NO:1024; SEQ ID NO:918 and SEQ ID NO: 1024; SEQ ID NO:919 and SEQ ID NO: 1024; SEQ ID NO:920 and SEQ ID NO: 1024; SEQ ID NO:921 and SEQ ID NO: 1024; SEQ ID NO:922 and SEQ ID NO: 1024; SEQ ID NO:923 and SEQ ID NO: 1024; SEQ ID NO:924 and SEQ ID NO: 1024; SEQ ID NO: 925 and SEQ ID NO: 1024; SEQ ID NO:926 and SEQ ID NO: 1024; SEQ ID NO:927 and SEQ ID NO:1024; SEQ ID NO:928 and SEQ ID NO: 1024; SEQ ID NO:929 and SEQ ID NO: 1024; SEQ ID NO:930 and SEQ ID NO:1024; SEQ ID NO:931 and SEQ ID NO: 1024; SEQ ID NO:932 and SEQ ID NO: 1024; SEQ ID NO:933 and SEQ ID NO:1024; SEQ ID NO:934 and SEQ ID NO:1024; SEQ ID NO:935 and SEQ ID NO:1024; SEQ ID NO: 936 and SEQ ID NO: 1024; SEQ ID NO:937 and SEQ ID NO: 1024; SEQ ID NO:938 and SEQ ID NO: 1024; SEQ ID NO:939 and SEQ ID NO: 1024; SEQ ID NO:940 and SEQ ID NO: 1024; SEQ ID NO:941 and SEQ ID NO: 1024; SEQ ID NO:942 and SEQ ID NO: 1024; SEQ ID NO:943 and SEQ ID NO:1024; SEQ ID NO:944 and SEQ ID NO:1024; SEQ ID NO:945 and SEQ ID NO: 1024; SEQ ID NO:946 and SEQ ID NO:1024; SEQ ID NO: 947 and SEQ ID NO: 1024; SEQ ID NO:948 and SEQ ID NO: 1024; SEQ ID NO:949 and SEQ ID NO:1024; SEQ ID NO:950 and SEQ ID NO:1024; SEQ ID NO:951 and SEQ ID NO: 1024; SEQ ID NO:952 and SEQ ID NO: 1024; SEQ ID NO:953 and SEQ ID NO: 1024; SEQ ID NO:954 and SEQ ID NO:1024; SEQ ID NO:955 and SEQ ID NO:1024; SEQ ID NO:956 and SEQ ID NO: 1024; SEQ ID NO:957 and SEQ ID NO: 1024; SEQ ID NO: 958 and SEQ ID NO: 1024; SEQ ID NO:959 and SEQ ID NO: 1024; SEQ ID NO:960 and SEQ ID NO: 1024; SEQ ID NO:961 and SEQ ID NO: 1024; SEQ ID NO:962 and SEQ ID NO: 1024; SEQ ID NO:963 and SEQ ID NO: 1024; SEQ ID NO:964 and SEQ ID NO: 1024; SEQ ID NO:965 and SEQ ID NO:1024; SEQ ID NO:966 and SEQ ID NO:1024; SEQ ID NO:967 and SEQ ID NO:1024; SEQ ID NO:968 and SEQ ID NO:1024; SEQ ID NO: 969 and SEQ ID NO: 1024; SEQ ID NO:970 and SEQ ID NO: 1024; SEQ ID NO:971 and SEQ ID NO: 1024; SEQ ID NO:972 and SEQ ID NO:1024; SEQ ID NO:973 and SEQ ID NO: 1024; SEQ ID NO:974 and SEQ ID NO: 1024; SEQ ID NO:975 and SEQ ID NO: 1024; SEQ ID NO:976 and SEQ ID NO: 1024; SEQ ID NO:977 and SEQ ID NO:1024; SEQ ID NO:978 and SEQ ID NO:1024; SEQ ID NO:979 and SEQ ID NO:1024; SEQ ID NO: 980 and SEQ ID NO: 1024; SEQ ID NO:981 and SEQ ID NO:1024; SEQ ID NO:982 and SEQ ID NO:1024; SEQ ID NO:983 and SEQ ID NO: 1024; SEQ ID NO:984 and SEQ ID NO: 1024; SEQ ID NO:985 and SEQ ID NO:1024; SEQ ID NO:986 and SEQ ID NO: 1024; SEQ ID NO:987 and SEQ ID NO:1024; SEQ ID NO:988 and SEQ ID NO:1024; SEQ ID NO:989 and SEQ ID NO:1024; SEQ ID NO:990 and SEQ ID NO:1024; SEQ ID NO: 991 and SEQ ID NO: 1024; SEQ ID NO:992 and SEQ ID NO: 1024; SEQ ID NO:993 and SEQ ID NO: 1024; SEQ ID NO:994 and SEQ ID NO: 1024; SEQ ID NO:995 and SEQ ID NO: 1024; SEQ ID NO:996 and SEQ ID NO: 1024; SEQ ID NO:997 and SEQ ID NO: 1024; SEQ ID NO:998 and SEQ ID NO:1024; SEQ ID NO:999 and SEQ ID NO:1024; SEQ ID NO: 1000 and SEQ ID NO:1024; SEQ ID NO: 1001 and SEQ ID NO: 1024; SEQ ID NO: 1002 and SEQ ID NO:1024; SEQ ID NO:1003 and SEQ ID NO: 1024; SEQ ID NO: 1004 and SEQ ID NO:1024; SEQ ID NO:1005 and SEQ ID NO: 1024; SEQ ID NO: 1006 and SEQ ID NO: 1024; SEQ ID NO: 1007 and SEQ ID NO: 1024; SEQ ID NO: 1008 and SEQ ID NO: 1024; SEQ ID NO: 1009 and SEQ ID NO: 1024; SEQ ID NO: 1010 and SEQ ID NO:1024; SEQ ID NO:1011 and SEQ ID NO:1024; SEQ ID NO: 1012 and SEQ ID NO:1024; SEQ ID NO:1013 and SEQ ID NO:1024; SEQ ID NO: 1014 and SEQ ID NO:1024; SEQ ID NO:1015 and SEQ ID NO:1024; SEQ ID NO: 1016 and SEQ ID NO:1024; SEQ ID NO:1017 and SEQ ID NO: 1024; SEQ ID NO: 1018 and SEQ ID NO: 1024; SEQ ID NO: 1019 and SEQ ID NO: 1024; and SEQ ID NO: 1020 and SEQ ID NO:1024.

In some aspects, the V_{H}-V_{L} pairs are selected from SEQ ID NO:854 and SEQ ID NO: 1025; SEQ ID NO:855 and SEQ ID NO:1025; SEQ ID NO:856 and SEQ ID NO:1025; SEQ ID NO:857 and SEQ ID NO:1025; SEQ ID NO:858 and SEQ ID NO: 1025; SEQ ID NO: 859 and SEQ ID NO: 1025; SEQ ID NO:860 and SEQ ID NO: 1025; SEQ ID NO:861 and SEQ ID NO:1025; SEQ ID NO:862 and SEQ ID NO:1025; SEQ ID NO:863 and SEQ ID NO: 1025; SEQ ID NO:864 and SEQ ID NO:1025; SEQ ID NO:865 and SEQ ID NO: 1025; SEQ ID NO:866 and SEQ ID NO:1025; SEQ ID NO:867 and SEQ ID NO:1025; SEQ ID NO:868 and SEQ ID NO: 1025; SEQ ID NO:869 and SEQ ID NO: 1025; SEQ ID NO: 870 and SEQ ID NO: 1025; SEQ ID NO:871 and SEQ ID NO:1025; SEQ ID NO:872 and SEQ ID NO:1025; SEQ ID NO:873 and SEQ ID NO: 1025; SEQ ID NO:874 and SEQ ID NO: 1025; SEQ ID NO: 875 and SEQ ID NO: 1025; SEQ ID NO:876 and SEQ ID NO: 1025; SEQ ID NO:877 and SEQ ID NO: 1025; SEQ ID NO:878 and SEQ ID NO:1025; SEQ ID NO:879 and SEQ ID NO: 1025; SEQ ID NO:880 and SEQ ID NO:1025; SEQ ID NO: 881 and SEQ ID NO:1025; SEQ ID NO:882 and SEQ ID NO:1025; SEQ ID NO:883 and SEQ ID NO:1025; SEQ ID NO:884 and SEQ ID NO:1025; SEQ ID NO:885 and SEQ ID NO: 1025; SEQ ID NO:886 and SEQ ID NO:1025; SEQ ID NO:887 and SEQ ID NO: 1025; SEQ ID NO:888 and SEQ ID NO:1025; SEQ ID NO:889 and SEQ ID NO:1025; SEQ ID NO:890 and SEQ ID NO: 1025; SEQ ID NO:891 and SEQ ID NO:1025; SEQ ID NO: 892 and SEQ ID NO: 1025; SEQ ID NO: 893 and SEQ ID NO:1025; SEQ ID NO:894 and SEQ ID NO: 1025; SEQ ID NO:895 and SEQ ID NO: 1025; SEQ ID NO:896 and SEQ ID NO: 1025; SEQ ID NO:897 and SEQ ID NO:1025; SEQ ID NO:898 and SEQ ID NO: 1025; SEQ ID NO:899 and SEQ ID NO: 1025; SEQ ID NO:900 and SEQ ID NO:1025; SEQ ID NO:901 and SEQ ID NO:1025; SEQ ID NO:902 and SEQ ID NO:1025; SEQ ID NO: 903 and SEQ ID NO: 1025; SEQ ID NO:904 and SEQ ID NO:1025; SEQ ID NO:905 and SEQ ID NO: 1025; SEQ ID NO:906 and SEQ ID NO: 1025; SEQ ID NO:907 and SEQ ID NO: 1025; SEQ ID NO:908 and SEQ ID NO: 1025; SEQ ID NO:909 and SEQ ID NO: 1025; SEQ ID NO:910 and SEQ ID NO:1025; SEQ ID NO:911 and SEQ ID NO: 1025; SEQ ID NO:912 and SEQ ID NO:1025; SEQ ID NO:913 and SEQ ID NO: 1025; SEQ ID NO: 914 and SEQ ID NO: 1025; SEQ ID NO:915 and SEQ ID NO:1025; SEQ ID NO:916 and SEQ ID NO: 1025; SEQ ID NO:917 and SEQ ID NO: 1025; SEQ ID NO:918 and SEQ ID NO: 1025; SEQ ID NO:919 and SEQ ID NO:1025; SEQ ID NO:920 and SEQ ID NO: 1025; SEQ ID NO:921 and SEQ ID NO: 1025; SEQ ID NO:922 and SEQ ID NO:1025; SEQ ID NO:923 and SEQ ID NO:1025; SEQ ID NO:924 and SEQ ID NO:1025; SEQ ID NO: 925 and SEQ ID NO: 1025; SEQ ID NO:926 and SEQ ID NO: 1025; SEQ ID NO:927 and SEQ ID NO: 1025; SEQ ID NO:928 and SEQ ID NO: 1025; SEQ ID NO:929 and SEQ ID NO: 1025; SEQ ID NO:930 and SEQ ID NO: 1025; SEQ ID NO:931 and SEQ ID NO: 1025; SEQ ID NO:932 and SEQ ID NO:1025; SEQ ID NO:933 and SEQ ID NO:1025; SEQ ID NO:934 and SEQ ID NO: 1025; SEQ ID NO:935 and SEQ ID NO: 1025; SEQ ID NO: 936 and SEQ ID NO: 1025; SEQ ID NO:937 and SEQ ID NO: 1025; SEQ ID NO:938 and SEQ ID NO: 1025; SEQ ID NO:939 and SEQ ID NO: 1025; SEQ ID NO:940 and SEQ ID NO: 1025; SEQ ID NO:941 and SEQ ID NO: 1025; SEQ ID NO:942 and SEQ ID NO: 1025; SEQ ID NO:943 and SEQ ID NO: 1025; SEQ ID NO:944 and SEQ ID NO:1025; SEQ ID NO:945 and SEQ ID NO:1025; SEQ ID NO:946 and SEQ ID NO: 1025; SEQ ID NO: 947 and SEQ ID NO: 1025; SEQ ID NO:948 and SEQ ID NO: 1025; SEQ ID NO:949 and SEQ ID NO:1025; SEQ ID NO:950 and SEQ ID NO: 1025; SEQ ID NO:951 and SEQ ID NO: 1025; SEQ ID NO:952 and SEQ ID NO: 1025; SEQ ID NO:953 and SEQ ID NO: 1025; SEQ ID NO:954 and SEQ ID NO:1025; SEQ ID NO:955 and SEQ ID NO:1025; SEQ ID NO:956 and SEQ ID NO: 1025; SEQ ID NO:957 and SEQ ID NO:1025; SEQ ID NO: 958 and SEQ ID NO: 1025; SEQ ID NO:959 and SEQ ID NO: 1025; SEQ ID NO:960 and SEQ ID NO:1025; SEQ ID NO:961 and SEQ ID NO:1025; SEQ ID NO:962 and SEQ ID NO: 1025; SEQ ID NO:963 and SEQ ID NO:1025; SEQ ID NO:964 and SEQ ID NO: 1025; SEQ ID NO:965 and SEQ ID NO:1025; SEQ ID NO:966 and SEQ ID NO:1025; SEQ ID NO:967 and SEQ ID NO: 1025; SEQ ID NO:968 and SEQ ID NO:1025; SEQ ID NO: 969 and SEQ ID NO: 1025; SEQ ID NO:970 and SEQ ID NO:1025; SEQ ID NO:971 and SEQ ID NO: 1025; SEQ ID NO:972 and SEQ ID NO: 1025; SEQ ID NO:973 and SEQ ID NO: 1025; SEQ ID NO:974 and SEQ ID NO: 1025; SEQ ID NO:975 and SEQ ID NO: 1025; SEQ ID NO:976 and SEQ ID NO: 1025; SEQ ID NO:977 and SEQ ID NO: 1025; SEQ ID NO:978 and SEQ ID NO:1025; SEQ ID NO:979 and SEQ ID NO: 1025; SEQ ID NO: 980 and SEQ ID NO: 1025; SEQ ID NO:981 and SEQ ID NO: 1025; SEQ ID NO:982 and SEQ ID NO:1025; SEQ ID NO:983 and SEQ ID NO: 1025; SEQ ID NO:984 and SEQ ID NO: 1025; SEQ ID NO:985 and SEQ ID NO: 1025; SEQ ID NO:986 and SEQ ID NO: 1025; SEQ ID NO:987 and SEQ ID NO: 1025; SEQ ID NO:988 and SEQ ID NO:1025; SEQ ID NO:989 and SEQ ID NO:1025; SEQ ID NO:990 and SEQ ID NO: 1025; SEQ ID NO: 991 and SEQ ID NO: 1025; SEQ ID NO:992 and SEQ ID NO: 1025; SEQ ID NO:993 and SEQ ID NO: 1025; SEQ ID NO:994 and SEQ ID NO: 1025; SEQ ID NO:995 and SEQ ID NO: 1025; SEQ ID NO:996 and SEQ ID NO: 1025; SEQ ID NO:997 and SEQ ID NO: 1025; SEQ ID NO:998 and SEQ ID NO:1025; SEQ ID NO:999 and SEQ ID NO:1025; SEQ ID NO: 1000 and SEQ ID NO:1025; SEQ ID NO:1001 and SEQ ID NO:1025; SEQ ID NO: 1002 and SEQ ID NO:1025; SEQ ID NO: 1003 and SEQ ID NO: 1025; SEQ ID NO: 1004 and SEQ ID NO: 1025; SEQ ID NO: 1005 and SEQ ID NO:1025; SEQ ID NO: 1006 and SEQ ID NO: 1025; SEQ ID NO: 1007 and SEQ ID NO: 1025; SEQ ID NO: 1008 and SEQ ID NO: 1025; SEQ ID NO: 1009 and SEQ ID NO:1025; SEQ ID NO: 1010 and SEQ ID NO: 1025; SEQ ID NO: 1011 and SEQ ID NO: 1025; SEQ ID NO: 1012 and SEQ ID NO:1025; SEQ ID NO:1013 and SEQ ID NO: 1025; SEQ ID NO: 1014 and SEQ ID NO:1025; SEQ ID NO:1015 and SEQ ID NO: 1025; SEQ ID NO: 1016 and SEQ ID NO:1025; SEQ ID NO:1017 and SEQ ID NO: 1025; SEQ ID NO: 1018 and SEQ ID NO:1025; SEQ ID NO: 1019 and SEQ ID NO:1025; and SEQ ID NO: 1020 and SEQ ID NO:1025.

In some aspects, the V_{H}-V_{L} pairs are selected from SEQ ID NO:854 and SEQ ID NO: 1026; SEQ ID NO:855 and SEQ ID NO:1026; SEQ ID NO:856 and SEQ ID NO:1026; SEQ ID NO:857 and SEQ ID NO:1026; SEQ ID NO:858 and SEQ ID NO:1026; SEQ ID NO: 859 and SEQ ID NO: 1026; SEQ ID NO:860 and SEQ ID NO: 1026; SEQ ID NO:861 and SEQ ID NO:1026; SEQ ID NO:862 and SEQ ID NO: 1026; SEQ ID NO:863 and SEQ ID NO: 1026; SEQ ID NO:864 and SEQ ID NO:1026; SEQ ID NO:865 and SEQ ID NO: 1026; SEQ ID NO:866 and SEQ ID NO:1026; SEQ ID NO:867 and SEQ ID NO:1026; SEQ ID NO:868 and SEQ ID NO: 1026; SEQ ID NO:869 and SEQ ID NO: 1026; SEQ ID NO: 870 and SEQ ID NO:1026; SEQ ID NO:871 and SEQ ID NO:1026; SEQ ID NO:872 and SEQ ID NO:1026; SEQ ID NO:873 and SEQ ID NO:1026; SEQ ID NO:874 and SEQ ID NO: 1026; SEQ ID NO:875 and SEQ ID NO: 1026; SEQ ID NO:876 and SEQ ID NO: 1026; SEQ ID NO:877 and SEQ ID NO:1026; SEQ ID NO:878 and SEQ ID NO:1026; SEQ ID NO:879 and SEQ ID NO:1026; SEQ ID NO:880 and SEQ ID NO:1026; SEQ ID NO: 881 and SEQ ID NO:1026; SEQ ID NO:882 and SEQ ID NO:1026; SEQ ID NO:883 and SEQ ID NO: 1026; SEQ ID NO:884 and SEQ ID NO:1026; SEQ ID NO:885 and SEQ ID NO: 1026; SEQ ID NO:886 and SEQ ID NO:1026; SEQ ID NO:887 and SEQ ID NO: 1026; SEQ ID NO:888 and SEQ ID NO:1026; SEQ ID NO:889 and SEQ ID NO:1026; SEQ ID NO:890 and SEQ ID NO: 1026; SEQ ID NO:891 and SEQ ID NO:1026; SEQ ID NO: 892 and SEQ ID NO: 1026; SEQ ID NO:893 and SEQ ID NO: 1026; SEQ ID NO:894 and SEQ ID NO:1026; SEQ ID NO:895 and SEQ ID NO:1026; SEQ ID NO:896 and SEQ ID NO: 1026; SEQ ID NO:897 and SEQ ID NO: 1026; SEQ ID NO:898 and SEQ ID NO: 1026; SEQ ID NO:899 and SEQ ID NO:1026; SEQ ID NO:900 and SEQ ID NO:1026; SEQ ID NO:901 and SEQ ID NO:1026; SEQ ID NO:902 and SEQ ID NO:1026; SEQ ID NO: 903 and SEQ ID NO: 1026; SEQ ID NO:904 and SEQ ID NO: 1026; SEQ ID NO:905 and SEQ ID NO: 1026; SEQ ID NO:906 and SEQ ID NO:1026; SEQ ID NO:907 and SEQ ID NO: 1026; SEQ ID NO:908 and SEQ ID NO:1026; SEQ ID NO:909 and SEQ ID NO: 1026; SEQ ID NO:910 and SEQ ID NO: 1026; SEQ ID NO:911 and SEQ ID NO: 1026; SEQ ID NO:912 and SEQ ID NO: 1026; SEQ ID NO:913 and SEQ ID NO:1026; SEQ ID NO: 914 and SEQ ID NO: 1026; SEQ ID NO:915 and SEQ ID NO: 1026; SEQ ID NO:916 and SEQ ID NO:1026; SEQ ID NO:917 and SEQ ID NO: 1026; SEQ ID NO:918 and SEQ ID NO: 1026; SEQ ID NO:919 and SEQ ID NO: 1026; SEQ ID NO:920 and SEQ ID NO: 1026; SEQ ID NO:921 and SEQ ID NO:1026; SEQ ID NO:922 and SEQ ID NO:1026; SEQ ID NO:923 and SEQ ID NO:1026; SEQ ID NO:924 and SEQ ID NO: 1026; SEQ ID NO: 925 and SEQ ID NO: 1026; SEQ ID NO:926 and SEQ ID NO: 1026; SEQ ID NO:927 and SEQ ID NO: 1026; SEQ ID NO:928 and SEQ ID NO: 1026; SEQ ID NO:929 and SEQ ID NO: 1026; SEQ ID NO:930 and SEQ ID NO:1026; SEQ ID NO:931 and SEQ ID NO: 1026; SEQ ID NO:932 and SEQ ID NO: 1026; SEQ ID NO:933 and SEQ ID NO:1026; SEQ ID NO:934 and SEQ ID NO:1026; SEQ ID NO:935 and SEQ ID NO:1026; SEQ ID NO: 936 and SEQ ID NO: 1026; SEQ ID NO:937 and SEQ ID NO: 1026; SEQ ID NO:938 and SEQ ID NO: 1026; SEQ ID NO:939 and SEQ ID NO: 1026; SEQ ID NO:940 and SEQ ID NO: 1026; SEQ ID NO:941 and SEQ ID NO: 1026; SEQ ID NO:942 and SEQ ID NO: 1026; SEQ ID NO:943 and SEQ ID NO:1026; SEQ ID NO:944 and SEQ ID NO:1026; SEQ ID NO:945 and SEQ ID NO: 1026; SEQ ID NO:946 and SEQ ID NO:1026; SEQ ID NO: 947 and SEQ ID NO: 1026; SEQ ID NO:948 and SEQ ID NO: 1026; SEQ ID NO:949 and SEQ ID NO:1026; SEQ ID NO:950 and SEQ ID NO: 1026; SEQ ID NO:951 and SEQ ID NO: 1026; SEQ ID NO:952 and SEQ ID NO: 1026; SEQ ID NO:953 and SEQ ID NO: 1026; SEQ ID NO:954 and SEQ ID NO: 1026; SEQ ID NO:955 and SEQ ID NO:1026; SEQ ID NO:956 and SEQ ID NO:1026; SEQ ID NO:957 and SEQ ID NO: 1026; SEQ ID NO: 958 and SEQ ID NO: 1026; SEQ ID NO:959 and SEQ ID NO: 1026; SEQ ID NO:960 and SEQ ID NO:1026; SEQ ID NO:961 and SEQ ID NO:1026; SEQ ID NO:962 and SEQ ID NO: 1026; SEQ ID NO:963 and SEQ ID NO:1026; SEQ ID NO:964 and SEQ ID NO: 1026; SEQ ID NO:965 and SEQ ID NO:1026; SEQ ID NO:966 and SEQ ID NO:1026; SEQ ID NO:967 and SEQ ID NO: 1026; SEQ ID NO:968 and SEQ ID NO:1026; SEQ ID NO: 969 and SEQ ID NO: 1026; SEQ ID NO:970 and SEQ ID NO: 1026; SEQ ID NO:971 and SEQ ID NO: 1026; SEQ ID NO:972 and SEQ ID NO:1026; SEQ ID NO:973 and SEQ ID NO: 1026; SEQ ID NO:974 and SEQ ID NO:1026; SEQ ID NO:975 and SEQ ID NO: 1026; SEQ ID NO:976 and SEQ ID NO: 1026; SEQ ID NO:977 and SEQ ID NO: 1026; SEQ ID NO:978 and SEQ ID NO: 1026; SEQ ID NO:979 and SEQ ID NO: 1026; SEQ ID NO: 980 and SEQ ID NO: 1026; SEQ ID NO:981 and SEQ ID NO:1026; SEQ ID NO:982 and SEQ ID NO: 1026; SEQ ID NO:983 and SEQ ID NO: 1026; SEQ ID NO:984 and SEQ ID NO: 1026; SEQ ID NO:985 and SEQ ID NO: 1026; SEQ ID NO:986 and SEQ ID NO: 1026; SEQ ID NO:987 and SEQ ID NO:1026; SEQ ID NO:988 and SEQ ID NO:1026; SEQ ID NO:989 and SEQ ID NO: 1026; SEQ ID NO:990 and SEQ ID NO: 1026; SEQ ID NO: 991 and SEQ ID NO: 1026; SEQ ID NO: 992 and SEQ ID NO: 1026; SEQ ID NO:993 and SEQ ID NO: 1026; SEQ ID NO:994 and SEQ ID NO: 1026; SEQ ID NO:995 and SEQ ID NO: 1026; SEQ ID NO:996 and SEQ ID NO:1026; SEQ ID NO:997 and SEQ ID NO: 1026; SEQ ID NO:998 and SEQ ID NO:1026; SEQ ID NO:999 and SEQ ID NO:1026; SEQ ID NO: 1000 and SEQ ID NO:1026; SEQ ID NO:1001 and SEQ ID NO: 1026; SEQ ID NO: 1002 and SEQ ID NO:1026; SEQ ID NO:1003 and SEQ ID NO:1026; SEQ ID NO: 1004 and SEQ ID NO:1026; SEQ ID NO:1005 and SEQ ID NO:1026; SEQ ID NO: 1006 and SEQ ID NO:1026; SEQ ID NO:1007 and SEQ ID NO:1026; SEQ ID NO: 1008 and SEQ ID NO:1026; SEQ ID NO: 1009 and SEQ ID NO: 1026; SEQ ID NO: 1010 and SEQ ID NO:1026; SEQ ID NO:1011 and SEQ ID NO: 1026; SEQ ID NO: 1012 and SEQ ID NO:1026; SEQ ID NO:1013 and SEQ ID NO:1026; SEQ ID NO: 1014 and SEQ ID NO:1026; SEQ ID NO:1015 and SEQ ID NO: 1026; SEQ ID NO: 1016 and SEQ ID NO:1026; SEQ ID NO:1017 and SEQ ID NO: 1026; SEQ ID NO: 1018 and SEQ ID NO: 1026; SEQ ID NO: 1019 and SEQ ID NO: 1026; and SEQ ID NO: 1020 and SEQ ID NO:1026.

5.7.4.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

### 5.7.5. Heavy Chain-Light Chain Pairs

In some embodiments, the antibody comprises a heavy chain sequence of an antibody disclosed herein and a light chain sequence of a suitable antibody. In some embodiments, the antibody comprises a heavy chain sequence of an antibody disclosed herein and a light chain sequence of an antibody disclosed herein.

In some embodiments, the heavy chain comprises a V_{H} sequence comprising, consisting of, or consisting essentially of any one of SEQ ID NOs: 854-1020, and the light chain comprises a light chain sequence of any suitable antibody. Techniques for determining whether a particular light chain will pair with a heavy chain as described herein are well known to those of skill in the art. For example, a cell-free protein synthesis reaction comprising a nucleic acid encoding the heavy chain of interest and a nucleic acid encoding the light chain to be assessed may be performed as described, for example, in Example 1.

In some embodiments, the heavy chain comprises a V_{H} sequence comprising, consisting of, or consisting essentially of any one of SEQ ID NOs: 854-1020, and the light chain comprises a V_{L} sequence comprising, consisting of, or consisting essentially of any one of SEQ ID NOs: 1021-1026.

In some embodiments, the heavy chain comprises a V_{H} sequence comprising, consisting of, or consisting essentially of any one of SEQ ID NOs: 979-1020, and the light chain comprises a V_{L} sequence comprising, consisting of, or consisting essentially of any one of SEQ ID NOs: 1021-1026.

In some embodiments, the heavy chain comprises a V_{H} sequence comprising, consisting of, or consisting essentially of any one of SEQ ID NOs: 979-1020, and the light chain comprises a V_{L} sequence comprising, consisting of, or consisting essentially of SEQ ID NO: 1022 or 1023.

In some embodiments, the antibody comprises a V_{H}/V_{L} pair together comprising, consisting of, or consisting essentially of the sequences of any pair selected from the group consisting of SEQ ID NOS: 854/1021, 855/1021, 856/1021, 857/1021, 858/1021, 859/1021, 860/1021, 861/1021, 862/1021, 863/1021, 864/1021, 865/1021, 866/1021, 867/1021, 868/1021, 869/1021, 870/1021, 871/1021, 872/1021, 873/1021, 874/1021, 875/1021, 876/1021, 877/1021, 878/1021, 879/1021, 880/1021, 881/1021, 882/1021, 883/1021, 884/1021, 885/1021, 886/1021, 887/1021, 888/1021, 889/1021, 890/1021, 891/1021, 892/1021, 893/1021, 894/1021, 895/1021, 896/1021, 897/1021, 898/1021, 899/1021, 900/1021, 901/1021, 902/1021, 903/1021, 904/1021, 905/1021, 906/1021, 907/1021, 908/1021, 909/1021, 910/1021, 911/1021, 912/1021, 913/1021, 914/1021, 915/1021, 916/1021, 917/1021, 918/1021, 919/1021, 920/1021, 921/1021, 922/1021, 923/1021, 924/1021, 925/1021, 926/1021, 927/1021, 928/1021, 929/1021, 930/1021, 931/1021, 932/1021, 933/1021, 934/1021, 935/1021, 936/1021, 937/1021, 938/1021, 939/1021, 940/1021, 941/1021, 942/1021, 943/1021, 944/1021, 945/1021, 946/1021, 947/1021, 948/1021, 949/1021, 950/1021, 951/1021, 952/1021, 953/1021, 954/1021, 955/1021, 956/1021, 957/1021, 958/1021, 959/1021, 960/1021, 961/1021, 962/1021, 963/1021, 964/1021, 965/1021, 966/1021, 967/1021, 968/1021, 969/1021, 970/1021, 971/1021, 972/1021, 973/1021, 974/1021, 975/1021, 976/1021, 977/1021, 978/1021, 979/1021, 980/1021, 981/1021, 982/1021, 983/1021, 984/1021, 985/1021, 986/1021, 987/1021, 988/1021, 989/1021, 990/1021, 991/1021, 992/1021, 993/1021, 994/1021, 995/1021, 996/1021, 997/1021, 998/1021, 999/1021, 1000/1021, 1001/1021, 1002/1021, 1003/1021, 1004/1021, 1005/1021, 1006/1021, 1007/1021, 1008/1021, 1009/1021, 1010/1021, 1011/1021, 1012/1021, 1013/1021, 1014/1021, 1015/1021, 1016/1021, 1017/1021, 1018/1021, 1019/1021, and 1020/1021.

In some embodiments, the antibody comprises a V_{H}/V_{L} pair together comprising, consisting of, or consisting essentially of the sequences of any pair selected from the group consisting of SEQ ID NOS: 854/1022, 855/1022, 856/1022, 857/1022, 858/1022, 859/1022, 860/1022, 861/1022, 862/1022, 863/1022, 864/1022, 865/1022, 866/1022, 867/1022, 868/1022, 869/1022, 870/1022, 871/1022, 872/1022, 873/1022, 874/1022, 875/1022, 876/1022, 877/1022, 878/1022, 879/1022, 880/1022, 881/1022, 882/1022, 883/1022, 884/1022, 885/1022, 886/1022, 887/1022, 888/1022, 889/1022, 890/1022, 891/1022, 892/1022, 893/1022, 894/1022, 895/1022, 896/1022, 897/1022, 898/1022, 899/1022, 900/1022, 901/1022, 902/1022, 903/1022, 904/1022, 905/1022, 906/1022, 907/1022, 908/1022, 909/1022, 910/1022, 911/1022, 912/1022, 913/1022, 914/1022, 915/1022, 916/1022, 917/1022, 918/1022, 919/1022, 920/1022, 921/1022, 922/1022, 923/1022, 924/1022, 925/1022, 926/1022, 927/1022, 928/1022, 929/1022, 930/1022, 931/1022, 932/1022, 933/1022, 934/1022, 935/1022, 936/1022, 937/1022, 938/1022, 939/1022, 940/1022, 941/1022, 942/1022, 943/1022, 944/1022, 945/1022, 946/1022, 947/1022, 948/1022, 949/1022, 950/1022, 951/1022, 952/1022, 953/1022, 954/1022, 955/1022, 956/1022, 957/1022, 958/1022, 959/1022, 960/1022, 961/1022, 962/1022, 963/1022, 964/1022, 965/1022, 966/1022, 967/1022, 968/1022, 969/1022, 970/1022, 971/1022, 972/1022, 973/1022, 974/1022, 975/1022, 976/1022, 977/1022, 978/1022, 979/1022, 980/1022, 981/1022, 982/1022, 983/1022, 984/1022, 985/1022, 986/1022, 987/1022, 988/1022, 989/1022, 990/1022, 991/1022, 992/1022, 993/1022, 994/1022, 995/1022, 996/1022, 997/1022, 998/1022, 999/1022, 1000/1022, 1001/1022, 1002/1022, 1003/1022, 1004/1022, 1005/1022, 1006/1022, 1007/1022, 1008/1022, 1009/1022, 1010/1022, 1011/1022, 1012/1022, 1013/1022, 1014/1022, 1015/1022, 1016/1022, 1017/1022, 1018/1022, 1019/1022, and 1020/1022.

In some embodiments, the antibody comprises a V_{H}/V_{L} pair together comprising, consisting of, or consisting essentially of the sequences of any pair selected from the group consisting of SEQ ID NOS: 854/1023, 855/1023, 856/1023, 857/1023, 858/1023, 859/1023, 860/1023, 861/1023, 862/1023, 863/1023, 864/1023, 865/1023, 866/1023, 867/1023, 868/1023, 869/1023, 870/1023, 871/1023, 872/1023, 873/1023, 874/1023, 875/1023, 876/1023, 877/1023, 878/1023, 879/1023, 880/1023, 881/1023, 882/1023, 883/1023, 884/1023, 885/1023, 886/1023, 887/1023, 888/1023, 889/1023, 890/1023, 891/1023, 892/1023, 893/1023, 894/1023, 895/1023, 896/1023, 897/1023, 898/1023, 899/1023, 900/1023, 901/1023, 902/1023, 903/1023, 904/1023, 905/1023, 906/1023, 907/1023, 908/1023, 909/1023, 910/1023, 911/1023, 912/1023, 913/1023, 914/1023, 915/1023, 916/1023, 917/1023, 918/1023, 919/1023, 920/1023, 921/1023, 922/1023, 923/1023, 924/1023, 925/1023, 926/1023, 927/1023, 928/1023, 929/1023, 930/1023, 931/1023, 932/1023, 933/1023, 934/1023, 935/1023, 936/1023, 937/1023, 938/1023, 939/1023, 940/1023, 941/1023, 942/1023, 943/1023, 944/1023, 945/1023, 946/1023, 947/1023, 948/1023, 949/1023, 950/1023, 951/1023, 952/1023, 953/1023, 954/1023, 955/1023, 956/1023, 957/1023, 958/1023, 959/1023, 960/1023, 961/1023, 962/1023, 963/1023, 964/1023, 965/1023, 966/1023, 967/1023, 968/1023, 969/1023, 970/1023, 971/1023, 972/1023, 973/1023, 974/1023, 975/1023, 976/1023, 977/1023, 978/1023, 979/1023, 980/1023, 981/1023, 982/1023, 983/1023, 984/1023, 985/1023, 986/1023, 987/1023, 988/1023, 989/1023, 990/1023, 991/1023, 992/1023, 993/1023, 994/1023, 995/1023, 996/1023, 997/1023, 998/1023, 999/1023, 1000/1023, 1001/1023, 1002/1023, 1003/1023, 1004/1023, 1005/1023, 1006/1023, 1007/1023, 1008/1023, 1009/1023, 1010/1023, 1011/1023, 1012/1023, 1013/1023, 1014/1023, 1015/1023, 1016/1023, 1017/1023, 1018/1023, 1019/1023, and 1020/1023.

### 5.8. Antibodies Comprising All Six CDRs

In some embodiments, the antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 4-170; the CDR-H2 sequence is a Chothia CDR-H2 sequence comprising, consisting of, or consisting essentially of SEQ ID NOs: 338-504; the CDR-H3 sequence is a CDR-H3 sequence comprising, consisting of, or consisting essentially of SEQ ID NOs: 672-838; the CDR-L1 sequence is a CDR-L1 sequence comprising, consisting of, or consisting essentially of SEQ ID NOs: 839-843; the CDR-L2 sequence is a CDR-L2 sequence comprising, consisting of, or consisting essentially of SEQ ID NOs: 844-848; and the CDR-L3 sequence is a CDR-L3 sequence comprising, consisting of, or consisting essentially of SEQ ID NOs: 849-853.

In some aspects, the CDR-H1 sequence is a Kabat CDR-H1 sequence comprising, consisting of, or consisting essentially of SEQ ID NOs: 171-337; the CDR-H2 sequence is a Kabat CDR-H2 sequence comprising, consisting of, or consisting essentially of SEQ ID NOs: 505-671; the CDR-H3 sequence is a CDR-H3 sequence comprising, consisting of, or consisting essentially of SEQ ID NOs: 672-838; the CDR-L1 sequence is a CDR-L1 sequence comprising, consisting of, or consisting essentially of SEQ ID NOs: 839-843; the CDR-L2 sequence is a CDR-L2 sequence comprising, consisting of, or consisting essentially of SEQ ID NOs: 844-848; and the CDR-L3 sequence is a CDR-L3 sequence comprising, consisting of, or consisting essentially of SEQ ID NOs: 849-853.

In some aspects, the antibody comprises three heavy chain CDRs from a V_{H} sequence selected from SEQ ID NOs: 854-1020, or variants thereof, and three light chain CDRs from a V_{L} sequence selected from SEQ ID NOs: 1021-1026, or variants thereof. The CDRs can be according to any CDR scheme known to the person of skill. In certain embodiments, the CDRs are Kabat CDRs. In certain embodiments, the CDRs are Chothia CDRs.

In some embodiments, the antibody comprises three heavy chain CDRs, or variants thereof, and three light chain CDRs, or variants thereof, from a V_{H}/V_{L} pair selected from the group consisting of SEQ ID NOS: 854/1021, 855/1021, 856/1021, 857/1021, 858/1021, 859/1021, 860/1021, 861/1021, 862/1021, 863/1021, 864/1021, 865/1021, 866/1021, 867/1021, 868/1021, 869/1021, 870/1021, 871/1021, 872/1021, 873/1021, 874/1021, 875/1021, 876/1021, 877/1021, 878/1021, 879/1021, 880/1021, 881/1021, 882/1021, 883/1021, 884/1021, 885/1021, 886/1021, 887/1021, 888/1021, 889/1021, 890/1021, 891/1021, 892/1021, 893/1021, 894/1021, 895/1021, 896/1021, 897/1021, 898/1021, 899/1021, 900/1021, 901/1021, 902/1021, 903/1021, 904/1021, 905/1021, 906/1021, 907/1021, 908/1021, 909/1021, 910/1021, 911/1021, 912/1021, 913/1021, 914/1021, 915/1021, 916/1021, 917/1021, 918/1021, 919/1021, 920/1021, 921/1021, 922/1021, 923/1021, 924/1021, 925/1021, 926/1021, 927/1021, 928/1021, 929/1021, 930/1021, 931/1021, 932/1021, 933/1021, 934/1021, 935/1021, 936/1021, 937/1021, 938/1021, 939/1021, 940/1021, 941/1021, 942/1021, 943/1021, 944/1021, 945/1021, 946/1021, 947/1021, 948/1021, 949/1021, 950/1021, 951/1021, 952/1021, 953/1021, 954/1021, 955/1021, 956/1021, 957/1021, 958/1021, 959/1021, 960/1021, 961/1021, 962/1021, 963/1021, 964/1021, 965/1021, 966/1021, 967/1021, 968/1021, 969/1021, 970/1021, 971/1021, 972/1021, 973/1021, 974/1021, 975/1021, 976/1021, 977/1021, 978/1021, 979/1021, 980/1021, 981/1021, 982/1021, 983/1021, 984/1021, 985/1021, 986/1021, 987/1021, 988/1021, 989/1021, 990/1021, 991/1021, 992/1021, 993/1021, 994/1021, 995/1021, 996/1021, 997/1021, 998/1021, 999/1021, 1000/1021, 1001/1021, 1002/1021, 1003/1021, 1004/1021, 1005/1021, 1006/1021, 1007/1021, 1008/1021, 1009/1021, 1010/1021, 1011/1021, 1012/1021, 1013/1021, 1014/1021, 1015/1021, 1016/1021, 1017/1021, 1018/1021, 1019/1021, and 1020/1021. The CDRs can be according to any CDR scheme known to the person of skill. In certain embodiments, the CDRs are Kabat CDRs. In certain embodiments, the CDRs are Chothia CDRs. In certain embodiments, the antibody further comprises the framework regions of the V_{H}/V_{L} pair.

In some embodiments, the antibody comprises three heavy chain CDRs, or variants thereof, and three light chain CDRs, or variants thereof, from a V_{H}/V_{L} pair selected from the group consisting of: 854/1022, 855/1022, 856/1022, 857/1022, 858/1022, 859/1022, 860/1022, 861/1022, 862/1022, 863/1022, 864/1022, 865/1022, 866/1022, 867/1022, 868/1022, 869/1022, 870/1022, 871/1022, 872/1022, 873/1022, 874/1022, 875/1022, 876/1022, 877/1022, 878/1022, 879/1022, 880/1022, 881/1022, 882/1022, 883/1022, 884/1022, 885/1022, 886/1022, 887/1022, 888/1022, 889/1022, 890/1022, 891/1022, 892/1022, 893/1022, 894/1022, 895/1022, 896/1022, 897/1022, 898/1022, 899/1022, 900/1022, 901/1022, 902/1022, 903/1022, 904/1022, 905/1022, 906/1022, 907/1022, 908/1022, 909/1022, 910/1022, 911/1022, 912/1022, 913/1022, 914/1022, 915/1022, 916/1022, 917/1022, 918/1022, 919/1022, 920/1022, 921/1022, 922/1022, 923/1022, 924/1022, 925/1022, 926/1022, 927/1022, 928/1022, 929/1022, 930/1022, 931/1022, 932/1022, 933/1022, 934/1022, 935/1022, 936/1022, 937/1022, 938/1022, 939/1022, 940/1022, 941/1022, 942/1022, 943/1022, 944/1022, 945/1022, 946/1022, 947/1022, 948/1022, 949/1022, 950/1022, 951/1022, 952/1022, 953/1022, 954/1022, 955/1022, 956/1022, 957/1022, 958/1022, 959/1022, 960/1022, 961/1022, 962/1022, 963/1022, 964/1022, 965/1022, 966/1022, 967/1022, 968/1022, 969/1022, 970/1022, 971/1022, 972/1022, 973/1022, 974/1022, 975/1022, 976/1022, 977/1022, 978/1022, 979/1022, 980/1022, 981/1022, 982/1022, 983/1022, 984/1022, 985/1022, 986/1022, 987/1022, 988/1022, 989/1022, 990/1022, 991/1022, 992/1022, 993/1022, 994/1022, 995/1022, 996/1022, 997/1022, 998/1022, 999/1022, 1000/1022, 1001/1022, 1002/1022, 1003/1022, 1004/1022, 1005/1022, 1006/1022, 1007/1022, 1008/1022, 1009/1022, 1010/1022, 1011/1022, 1012/1022, 1013/1022, 1014/1022, 1015/1022, 1016/1022, 1017/1022, 1018/1022, 1019/1022, and 1020/1022. The CDRs can be according to any CDR scheme known to the person of skill. In certain embodiments, the CDRs are Kabat CDRs. In certain embodiments, the CDRs are Chothia CDRs. In certain embodiments, the antibody further comprises the framework regions of the V_{H}/V_{L} pair.

In some embodiments, the antibody comprises three heavy chain CDRs, or variants thereof, and three light chain CDRs, or variants thereof, from a V_{H}/V_{L} pair selected from the group consisting of: 854/1023, 855/1023, 856/1023, 857/1023, 858/1023, 859/1023, 860/1023, 861/1023, 862/1023, 863/1023, 864/1023, 865/1023, 866/1023, 867/1023, 868/1023, 869/1023, 870/1023, 871/1023, 872/1023, 873/1023, 874/1023, 875/1023, 876/1023, 877/1023, 878/1023, 879/1023, 880/1023, 881/1023, 882/1023, 883/1023, 884/1023, 885/1023, 886/1023, 887/1023, 888/1023, 889/1023, 890/1023, 891/1023, 892/1023, 893/1023, 894/1023, 895/1023, 896/1023, 897/1023, 898/1023, 899/1023, 900/1023, 901/1023, 902/1023, 903/1023, 904/1023, 905/1023, 906/1023, 907/1023, 908/1023, 909/1023, 910/1023, 911/1023, 912/1023, 913/1023, 914/1023, 915/1023, 916/1023, 917/1023, 918/1023, 919/1023, 920/1023, 921/1023, 922/1023, 923/1023, 924/1023, 925/1023, 926/1023, 927/1023, 928/1023, 929/1023, 930/1023, 931/1023, 932/1023, 933/1023, 934/1023, 935/1023, 936/1023, 937/1023, 938/1023, 939/1023, 940/1023, 941/1023, 942/1023, 943/1023, 944/1023, 945/1023, 946/1023, 947/1023, 948/1023, 949/1023, 950/1023, 951/1023, 952/1023, 953/1023, 954/1023, 955/1023, 956/1023, 957/1023, 958/1023, 959/1023, 960/1023, 961/1023, 962/1023, 963/1023, 964/1023, 965/1023, 966/1023, 967/1023, 968/1023, 969/1023, 970/1023, 971/1023, 972/1023, 973/1023, 974/1023, 975/1023, 976/1023, 977/1023, 978/1023, 979/1023, 980/1023, 981/1023, 982/1023, 983/1023, 984/1023, 985/1023, 986/1023, 987/1023, 988/1023, 989/1023, 990/1023, 991/1023, 992/1023, 993/1023, 994/1023, 995/1023, 996/1023, 997/1023, 998/1023, 999/1023, 1000/1023, 1001/1023, 1002/1023, 1003/1023, 1004/1023, 1005/1023, 1006/1023, 1007/1023, 1008/1023, 1009/1023, 1010/1023, 1011/1023, 1012/1023, 1013/1023, 1014/1023, 1015/1023, 1016/1023, 1017/1023, 1018/1023, 1019/1023, and 1020/1023.

In some embodiments, the antibody comprises three Chothia heavy chain CDRs according to one of the following groups of heavy chain CDR SEQ ID NOs: 4/338/672; 5/339/673; 6/340/674; 7/341/675; 8/342/676; 9/343/677; 10/344/678; 11/345/679; 12/346/680; 13/347/681; 14/348/682; 15/349/683; 16/350/684; 17/351/685; 18/352/686; 19/353/687; 20/354/688; 21/355/689; 22/356/690; 23/357/691; 24/358/692; 25/359/693; 26/360/694; 27/361/695; 28/362/696; 29/363/697; 30/364/698; 31/365/699; 32/366/700; 33/367/701; 34/368/702; 35/369/703; 36/370/704; 37/371/705; 38/372/706; 39/373/707; 40/374/708; 41/375/709; 42/376/710; 43/377/711; 44/378/712; 45/379/713; 46/380/714; 47/381/715; 48/382/716; 49/383/717; 50/384/718; 51/385/719; 52/386/720; 53/387/721; 54/388/722; 55/389/723; 56/390/724; 57/391/725; 58/392/726; 59/393/727; 60/394/728; 61/395/729; 62/396/730; 63/397/731; 64/398/732; 65/399/733; 66/400/734; 67/401/735; 68/402/736; 69/403/737; 70/404/738; 71/405/739; 72/406/740; 73/407/741; 74/408/742; 75/409/743; 76/410/744; 77/411/745; 78/412/746; 79/413/747; 80/414/748; 81/415/749; 82/416/750; 83/417/751; 84/418/752; 85/419/753; 86/420/754; 87/421/755; 88/422/756; 89/423/757; 90/424/758; 91/425/759; 92/426/760; 93/427/761; 94/428/762; 95/429/763; 96/430/764; 97/431/765; 98/432/766; 99/433/767; 100/434/768; 101/435/769; 102/436/770; 103/437/771; 104/438/772; 105/439/773; 106/440/774; 107/441/775; 108/442/776; 109/443/777; 110/444/778; 111/445/779; 112/446/780; 113/447/781; 114/448/782; 115/449/783; 116/450/784; 117/451/785; 118/452/786; 119/453/787; 120/454/788; 121/455/789; 122/456/790; 123/457/791; 124/458/792; 125/459/793; 126/460/794; 127/461/795; 128/462/796; 129/463/797; 130/464/798; 131/465/799; 132/466/800; 133/467/801; 134/468/802; 135/469/803; 136/470/804; 137/471/805; 138/472/806; 139/473/807; 140/474/808; 141/475/809; 142/476/810; 143/477/811; 144/478/812; 145/479/813; 146/480/814; 147/481/815; 148/482/816; 149/483/817; 150/484/818; 151/485/819; 152/486/820; 153/487/821; 154/488/822; 155/489/823; 156/490/824; 157/491/825; 158/492/826; 159/493/827; 160/494/828; 161/495/829; 162/496/830; 163/497/831; 164/498/832; 165/499/833; 166/500/834; 167/501/835; 168/502/836; 169/503/837; and 170/504/838.

In some embodiments, the antibody comprises six CDRs according to one of the following groups of CDR SEQ ID NOs: 4/338/672/839/844/849; 5/339/673/839/844/849; 6/340/674/839/844/849; 7/341/675/839/844/849; 8/342/676/839/844/849; 9/343/677/839/844/849; 10/344/678/839/844/849; 11/345/679/839/844/849; 12/346/680/839/844/849; 13/347/681/839/844/849; 14/348/682/839/844/849; 15/349/683/839/844/849; 16/350/684/839/844/849; 17/351/685/839/844/849; 18/352/686/839/844/849; 19/353/687/839/844/849; 20/354/688/839/844/849; 21/355/689/839/844/849; 22/356/690/839/844/849; 23/357/691/839/844/849; 24/358/692/839/844/849; 25/359/693/839/844/849; 26/360/694/839/844/849; 27/361/695/839/844/849; 28/362/696/839/844/849; 29/363/697/839/844/849; 30/364/698/839/844/849; 31/365/699/839/844/849; 32/366/700/839/844/849; 33/367/701/839/844/849; 34/368/702/839/844/849; 35/369/703/839/844/849; 36/370/704/839/844/849; 37/371/705/839/844/849; 38/372/706/839/844/849; 39/373/707/839/844/849; 40/374/708/839/844/849; 41/375/709/839/844/849; 42/376/710/839/844/849; 43/377/711/839/844/849; 44/378/712/839/844/849; 45/379/713/839/844/849; 46/380/714/839/844/849; 47/381/715/839/844/849; 48/382/716/839/844/849; 49/383/717/839/844/849; 50/384/718/839/844/849; 51/385/719/839/844/849; 52/386/720/839/844/849; 53/387/721/839/844/849; 54/388/722/839/844/849; 55/389/723/839/844/849; 56/390/724/839/844/849; 57/391/725/839/844/849; 58/392/726/839/844/849; 59/393/727/839/844/849; 60/394/728/839/844/849; 61/395/729/839/844/849; 62/396/730/839/844/849; 63/397/731/839/844/849; 64/398/732/839/844/849; 65/399/733/839/844/849; 66/400/734/839/844/849; 67/401/735/839/844/849; 68/402/736/839/844/849; 69/403/737/839/844/849; 70/404/738/839/844/849; 71/405/739/839/844/849; 72/406/740/839/844/849; 73/407/741/839/844/849; 74/408/742/839/844/849; 75/409/743/839/844/849; 76/410/744/839/844/849; 77/411/745/839/844/849; 78/412/746/839/844/849; 79/413/747/839/844/849; 80/414/748/839/844/849; 81/415/749/839/844/849; 82/416/750/839/844/849; 83/417/751/839/844/849; 84/418/752/839/844/849; 85/419/753/839/844/849; 86/420/754/839/844/849; 87/421/755/839/844/849; 88/422/756/839/844/849; 89/423/757/839/844/849; 90/424/758/839/844/849; 91/425/759/839/844/849; 92/426/760/839/844/849; 93/427/761/839/844/849; 94/428/762/839/844/849; 95/429/763/839/844/849; 96/430/764/839/844/849; 97/431/765/839/844/849; 98/432/766/839/844/849; 99/433/767/839/844/849; 100/434/768/839/844/849; 101/435/769/839/844/849; 102/436/770/839/844/849; 103/437/771/839/844/849; 104/438/772/839/844/849; 105/439/773/839/844/849; 106/440/774/839/844/849; 107/441/775/839/844/849; 108/442/776/839/844/849; 109/443/777/839/844/849; 110/444/778/839/844/849; 111/445/779/839/844/849; 112/446/780/839/844/849; 113/447/781/839/844/849; 114/448/782/839/844/849; 115/449/783/839/844/849; 116/450/784/839/844/849; 117/451/785/839/844/849; 118/452/786/839/844/849; 119/453/787/839/844/849; 120/454/788/839/844/849; 121/455/789/839/844/849; 122/456/790/839/844/849; 123/457/791/839/844/849; 124/458/792/839/844/849; 125/459/793/839/844/849; 126/460/794/839/844/849; 127/461/795/839/844/849; 128/462/796/839/844/849; 129/463/797/839/844/849; 130/464/798/839/844/849; 131/465/799/839/844/849; 132/466/800/839/844/849; 133/467/801/839/844/849; 134/468/802/839/844/849; 135/469/803/839/844/849; 136/470/804/839/844/849; 137/471/805/839/844/849; 138/472/806/839/844/849; 139/473/807/839/844/849; 140/474/808/839/844/849; 141/475/809/839/844/849; 142/476/810/839/844/849; 143/477/811/839/844/849; 144/478/812/839/844/849; 145/479/813/840/845/850; 146/480/814/840/845/850; 147/481/815/840/845/850; 148/482/816/840/845/850; 149/483/817/840/845/850; 150/484/818/840/845/850; 151/485/819/840/845/850; 152/486/820/840/845/850; 153/487/821/840/845/850; 154/488/822/840/845/850; 155/489/823/840/845/850; 156/490/824/840/845/850; 157/491/825/840/845/850; 158/492/826/840/845/850; 159/493/827/840/845/850; 160/494/828/840/845/850; 161/495/829/840/845/850; 162/496/830/840/845/850; 163/497/831/840/845/850; 164/498/832/840/845/850; 165/499/833/840/845/850; 166/500/834/840/845/850; 167/501/835/840/845/850; 168/502/836/840/845/850; 169/503/837/840/845/850; 170/504/838/840/845/850; 145/479/813/841/846/851; 146/480/814/841/846/851; 147/481/815/841/846/851; 148/482/816/841/846/851; 149/483/817/841/846/851; 150/484/818/841/846/851; 151/485/819/841/846/851; 152/486/820/841/846/851; 153/487/821/841/846/851; 154/488/822/841/846/851; 155/489/823/841/846/851; 156/490/824/841/846/851; 157/491/825/841/846/851; 158/492/826/841/846/851; 159/493/827/841/846/851; 160/494/828/841/846/851; 161/495/829/841/846/851; 162/496/830/841/846/851; 163/497/831/841/846/851; 164/498/832/841/846/851; 165/499/833/841/846/851; 166/500/834/841/846/851; 167/501/835/841/846/851; 168/502/836/841/846/851; 169/503/837/841/846/851; and 170/504/838/841/846/851.

In some embodiments, the isolated antibody comprises three Kabat HC CDRs according to one of the following groups of HC CDR SEQ ID NOs: 171/505/672; 172/506/673; 173/507/674; 174/508/675; 175/509/676; 176/510/677; 177/511/678; 178/512/679; 179/513/680; 180/514/681; 181/515/682; 182/516/683; 183/517/684; 184/518/685; 185/519/686; 186/520/687; 187/521/688; 188/522/689; 189/523/690; 190/524/691; 191/525/692; 192/526/693; 193/527/694; 194/528/695; 195/529/696; 196/530/697; 197/531/698; 198/532/699; 199/533/700; 200/534/701; 201/535/702; 202/536/703; 203/537/704; 204/538/705; 205/539/706; 206/540/707; 207/541/708; 208/542/709; 209/543/710; 210/544/711; 211/545/712; 212/546/713; 213/547/714; 214/548/715; 215/549/716; 216/550/717; 217/551/718; 218/552/719; 219/553/720; 220/554/721; 221/555/722; 222/556/723; 223/557/724; 224/558/725; 225/559/726; 226/560/727; 227/561/728; 228/562/729; 229/563/730; 230/564/731; 231/565/732; 232/566/733; 233/567/734; 234/568/735; 235/569/736; 236/570/737; 237/571/738; 238/572/739; 239/573/740; 240/574/741; 241/575/742; 242/576/743; 243/577/744; 244/578/745; 245/579/746; 246/580/747; 247/581/748; 248/582/749; 249/583/750; 250/584/751; 251/585/752; 252/586/753; 253/587/754; 254/588/755; 255/589/756; 256/590/757; 257/591/758; 258/592/759; 259/593/760; 260/594/761; 261/595/762; 262/596/763; 263/597/764; 264/598/765; 265/599/766; 266/600/767; 267/601/768; 268/602/769; 269/603/770; 270/604/771; 271/605/772; 272/606/773; 273/607/774; 274/608/775; 275/609/776; 276/610/777; 277/611/778; 278/612/779; 279/613/780; 280/614/781; 281/615/782; 282/616/783; 283/617/784; 284/618/785; 285/619/786; 286/620/787; 287/621/788; 288/622/789; 289/623/790; 290/624/791; 291/625/792; 292/626/793; 293/627/794; 294/628/795; 295/629/796; 296/630/797; 297/631/798; 298/632/799; 299/633/800; 300/634/801; 301/635/802; 302/636/803; 303/637/804; 304/638/805; 305/639/806; 306/640/807; 307/641/808; 308/642/809; 309/643/810; 310/644/811; 311/645/812; 312/646/813; 313/647/814; 314/648/815; 315/649/816; 316/650/817; 317/651/818; 318/652/819; 319/653/820; 320/654/821; 321/655/822; 322/656/823; 323/657/824; 324/658/825; 325/659/826; 326/660/827; 327/661/828; 328/662/829; 329/663/830; 330/664/831; 331/665/832; 332/666/833; 333/667/834; 334/668/835; 335/669/836; 336/670/837; and 337/671/838.

In some embodiments, the isolated antibody comprises six CDRs according to one of the following groups of CDR SEQ ID NOs: 171/505/672/839/844/849; 172/506/673/839/844/849; 173/507/674/839/844/849; 174/508/675/839/844/849; 175/509/676/839/844/849; 176/510/677/839/844/849; 177/511/678/839/844/849; 178/512/679/839/844/849; 179/513/680/839/844/849; 180/514/681/839/844/849; 181/515/682/839/844/849; 182/516/683/839/844/849; 183/517/684/839/844/849; 184/518/685/839/844/849; 185/519/686/839/844/849; 186/520/687/839/844/849; 187/521/688/839/844/849; 188/522/689/839/844/849; 189/523/690/839/844/849; 190/524/691/839/844/849; 191/525/692/839/844/849; 192/526/693/839/844/849; 193/527/694/839/844/849; 194/528/695/839/844/849; 195/529/696/839/844/849; 196/530/697/839/844/849; 197/531/698/839/844/849; 198/532/699/839/844/849; 199/533/700/839/844/849; 200/534/701/839/844/849; 201/535/702/839/844/849; 202/536/703/839/844/849; 203/537/704/839/844/849; 204/538/705/839/844/849; 205/539/706/839/844/849; 206/540/707/839/844/849; 207/541/708/839/844/849; 208/542/709/839/844/849; 209/543/710/839/844/849; 210/544/711/839/844/849; 211/545/712/839/844/849; 212/546/713/839/844/849; 213/547/714/839/844/849; 214/548/715/839/844/849; 215/549/716/839/844/849; 216/550/717/839/844/849; 217/551/718/839/844/849; 218/552/719/839/844/849; 219/553/720/839/844/849; 220/554/721/839/844/849; 221/555/722/839/844/849; 222/556/723/839/844/849; 223/557/724/839/844/849; 224/558/725/839/844/849; 225/559/726/839/844/849; 226/560/727/839/844/849; 227/561/728/839/844/849; 228/562/729/839/844/849; 229/563/730/839/844/849; 230/564/731/839/844/849; 231/565/732/839/844/849; 232/566/733/839/844/849; 233/567/734/839/844/849; 234/568/735/839/844/849; 235/569/736/839/844/849; 236/570/737/839/844/849; 237/571/738/839/844/849; 238/572/739/839/844/849; 239/573/740/839/844/849; 240/574/741/839/844/849; 241/575/742/839/844/849; 242/576/743/839/844/849; 243/577/744/839/844/849; 244/578/745/839/844/849; 245/579/746/839/844/849; 246/580/747/839/844/849; 247/581/748/839/844/849; 248/582/749/839/844/849; 249/583/750/839/844/849; 250/584/751/839/844/849; 251/585/752/839/844/849; 252/586/753/839/844/849; 253/587/754/839/844/849; 254/588/755/839/844/849; 255/589/756/839/844/849; 256/590/757/839/844/849; 257/591/758/839/844/849; 258/592/759/839/844/849; 259/593/760/839/844/849; 260/594/761/839/844/849; 261/595/762/839/844/849; 262/596/763/839/844/849; 263/597/764/839/844/849; 264/598/765/839/844/849; 265/599/766/839/844/849; 266/600/767/839/844/849; 267/601/768/839/844/849; 268/602/769/839/844/849; 269/603/770/839/844/849; 270/604/771/839/844/849; 271/605/772/839/844/849; 272/606/773/839/844/849; 273/607/774/839/844/849; 274/608/775/839/844/849; 275/609/776/839/844/849; 276/610/777/839/844/849; 277/611/778/839/844/849; 278/612/779/839/844/849; 279/613/780/839/844/849; 280/614/781/839/844/849; 281/615/782/839/844/849; 282/616/783/839/844/849; 283/617/784/839/844/849; 284/618/785/839/844/849; 285/619/786/839/844/849; 286/620/787/839/844/849; 287/621/788/839/844/849; 288/622/789/839/844/849; 289/623/790/839/844/849; 290/624/791/839/844/849; 291/625/792/839/844/849; 292/626/793/839/844/849; 293/627/794/839/844/849; 294/628/795/839/844/849; 295/629/796/839/844/849; 296/630/797/839/844/849; 297/631/798/839/844/849; 298/632/799/839/844/849; 299/633/800/839/844/849; 300/634/801/839/844/849; 301/635/802/839/844/849; 302/636/803/839/844/849; 303/637/804/839/844/849; 304/638/805/839/844/849; 305/639/806/839/844/849; 306/640/807/839/844/849; 307/641/808/839/844/849; 308/642/809/839/844/849; 309/643/810/839/844/849; 310/644/811/839/844/849; 311/645/812/840/845/850; 312/646/813/840/845/850; 313/647/814/840/845/850; 314/648/815/840/845/850; 315/649/816/840/845/850; 316/650/817/840/845/850; 317/651/818/840/845/850; 318/652/819/840/845/850; 319/653/820/840/845/850; 320/654/821/840/845/850; 321/655/822/840/845/850; 322/656/823/840/845/850; 323/657/824/840/845/850; 324/658/825/840/845/850; 325/659/826/840/845/850; 326/660/827/840/845/850; 327/661/828/840/845/850; 328/662/829/840/845/850; 329/663/830/840/845/850; 330/664/831/840/845/850; 331/665/832/840/845/850; 332/666/833/840/845/850; 333/667/834/840/845/850; 334/668/835/840/845/850; 335/669/836/840/845/850; 336/670/837/840/845/850; 337/671/838/840/845/850; 311/645/812/841/846/851; 312/646/813/841/846/851; 313/647/814/841/846/851; 314/648/815/841/846/851; 315/649/816/841/846/851; 316/650/817/841/846/851; 317/651/818/841/846/851; 318/652/819/841/846/851; 319/653/820/841/846/851; 320/654/821/841/846/851; 321/655/822/841/846/851; 322/656/823/841/846/851; 323/657/824/841/846/851; 324/658/825/841/846/851; 325/659/826/841/846/851; 326/660/827/841/846/851; 327/661/828/841/846/851; 328/662/829/841/846/851; 329/663/830/841/846/851; 330/664/831/841/846/851; 331/665/832/841/846/851; 332/666/833/841/846/851; 333/667/834/841/846/851; 334/668/835/841/846/851; 335/669/836/841/846/851; 336/670/837/841/846/851; and 337/671/838/841/846/851.

In certain embodiments, the CDRs can be according to any CDR scheme known to the person of skill. In certain embodiments, the CDRs are Kabat CDRs. In certain embodiments, the CDRs are Chothia CDRs. In certain embodiments, the antibody further comprises the framework regions of the V_{H}/V_{L} pair.

### 5.8.1. Variants of Antibodies Comprising All Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 95%, 96%, 97%, 98%, or 99% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, 95%, 96%, 97%, 98%, or 99% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, 95%, 96%, 97%, 98%, or 99% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, 95%, 96%, 97%, 98%, or 99% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, 95%, 96%, 97%, 98%, or 99% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, 95%, 96%, 97%, 98%, or 99% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 6. Germline

In some embodiments, the antibody or antibody of the conjugate that specifically binds ROR1 is an antibody comprising a variable region that is encoded by a particular germline gene, or a variant thereof. The illustrative antibodies provided herein comprise variable regions that are encoded by the heavy chain variable region germline genes VH1-18, VH3-33, VH2-5, VH2-70, and VH4-30-4. or variants thereof; and the light chain variable region germline genes Vκ1-5, Vκ3-11, Vκ2-20, Vκ1-33, and Vκ1-16, or variants thereof.

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H}1, V_{H}2, V_{H}3, or V_{H}4 families, or a variant thereof. In some embodiments, a CDR-L sequence provided herein may be combined with a variable region encoded by a variable region germline gene selected from the Vκ1, Vκ2, or Vκ3, or a variant thereof.

### 7. Affinity

In some embodiments, the affinity of the antibody for ROR1 as indicated by K_{D}, is less than about 10⁻⁵ M, less than about 10⁻⁶ M, less than about 10⁻⁷ M, less than about 10⁻⁸ M, less than about 10⁻⁹ M, or less than about 10⁻¹⁰ M. In some embodiments, the affinity of the antibody is between about 10⁻⁷ M and 10⁻¹¹ M. In some embodiments, the affinity of the antibody is between about 10⁻⁷ M and 10⁻¹⁰ M. In some embodiments, the affinity of the antibody is between about 10⁻⁷ M and 10⁻⁹ M. In some embodiments, the affinity of the antibody is between about 10⁻⁷ M and 10⁻⁸ M. In some embodiments, the affinity of the antibody is between about 10⁻⁸ M and 10⁻¹¹ M. In some embodiments, the affinity of the antibody is between about 10⁻⁸ M and 10⁻¹⁰ M. In some embodiments, the affinity of the antibody is between about 10⁻⁹ M and 10⁻¹¹ M. In some embodiments, the affinity of the antibody is between about 10⁻⁹ M and 10⁻¹⁰ M.

In some embodiments, the affinity of the antibody for human ROR1, human ROR1 extracellular domain, or for individual domains within human ROR1, as determined by surface plasmon resonance at 25° C., and as indicated by K_{D}, is from about 1.39×10⁻¹⁰ M to about 1.01×10⁻⁷ M. In some embodiments, the affinity of the antibody for human ROR1, as determined by surface plasmon resonance at 25° C., and as indicated by K_{D}, is from about 5.04×10⁻¹⁰ M to about 9.76×10⁻⁸ M. In some embodiments, the affinity of the antibody for human ROR1, as determined by surface plasmon resonance at 25° C., and as indicated by K_{D}, is from about 1.06×10⁻⁹ M to about 2.45×10⁻⁸ M. In some embodiments, the affinity of the antibody for human ROR1 is about any of the K_{D} values reported for human ROR1 in the examples below.

In some embodiments the antibody has a kₐ of at least about 10⁴ M⁻¹×sec⁻¹. In some embodiments the antibody has a kₐ of at least about 105 M⁻¹×sec⁻¹. In some embodiments the antibody has a kₐ of at least about 106 M⁻¹×sec⁻¹. In some embodiments the antibody has a kₐ of between about 104 M⁻¹×sec⁻¹ and about 107 M⁻¹×sec⁻¹. In some embodiments the antibody has a kₐ of between about 105 M⁻¹×sec⁻¹ and about 107 M⁻¹×sec-1. In some embodiments the antibody has a kₐ of between about 106 M⁻¹×sec⁻¹ and about 107 M⁻¹×sec⁻¹.

In some embodiments the antibody has a kₐ when associating with human ROR1, human ROR1 extracellular domain, or for individual domains within human ROR1, as determined by surface plasmon resonance at 25° C., of from about 9.17×10⁴ M⁻¹×sec⁻¹ to about 5.94×10⁶ M⁻¹×sec⁻¹. In some embodiments the antibody has a kₐ when associating with human ROR1, as determined by surface plasmon resonance at 25° C., of from about 1.71×10⁵ M⁻¹×sec⁻¹ to about 1.17×10⁶ M⁻¹×sec⁻¹. In some embodiments the antibody has a kₐ when associating with human ROR1, as determined by surface plasmon resonance at 25° C., of from about 2.56×10⁵ M⁻¹×sec⁻¹ to about 7.75×10⁵ M⁻¹×sec⁻¹. In some embodiments the antibody has a kₐ when associating with human ROR1 of about any of the kₐ values reported for human ROR1 in the examples below.

In some embodiments the antibody has a k_{d} of about 10⁻⁵ sec⁻¹ or less. In some embodiments the antibody has a k_{d} of about 10⁻⁴ sec⁻¹ or less. In some embodiments the antibody has a k_{d} of about 10⁻³ sec⁻¹ or less. In some embodiments the antibody has a k_{d} of between about 10⁻² sec⁻¹ and about 10⁻⁵ sec⁻¹. In some embodiments the antibody has a k_{d} of between about 10⁻² sec⁻¹ and about 104 sec⁻¹. In some embodiments the antibody has a k_{d} of between about 10⁻³ sec⁻¹ and about 10⁻⁵ sec⁻¹.

In some embodiments the antibody has a k_{d} when dissociating from human ROR1, human ROR1 extracellular domain, or for individual domains within human ROR1, as determined by surface plasmon resonance at 25° C., of from about 1.01×10⁻⁴ sec⁻¹ to about 0.05 sec⁻¹. In some embodiments the antibody has a k_{d} when dissociating from human ROR1, as determined by surface plasmon resonance at 25° C., of from about 2.27×10⁻⁴ sec⁻¹ to about 0.01 sec⁻¹. In some embodiments the antibody has a k_{d} when dissociating from human ROR1, as determined by surface plasmon resonance at 25° C., of from about 5.05×10⁻⁴ sec⁻¹ to about 8.50×10−3 sec⁻¹. In some embodiments the antibody has a k_{d} when dissociating from human ROR1 of about any of the k_{d} values reported for human ROR1 in the examples below.

In some aspects, the K_{D}, kₐ, and k_{d} are determined at 25° C. In some embodiments, the K_{D}, kₐ, and k_{d} are determined by surface plasmon resonance. In some embodiments, the K_{D}, kₐ, and k_{d} are determined according to the methods described in the Examples provided herein.

### 8. Epitope Bins

In some embodiments, the antibody binds the same epitope as an antibody encompassing any of SEQ ID NOs: 854-1020. In some embodiments, the antibody binds the same epitope as an antibody comprising any of the V_{H}-V_{L} pairs, above. In some embodiments, the antibody competes for epitope binding with an antibody encompassing any of SEQ ID NOs: 854-1020. In some embodiments, the antibody competes for epitope binding with an antibody comprising any of the V_{H}-V_{L} pairs, above.

### 9. Glycosylation Variants

In certain embodiments, an antibody may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

### 10. Fc Variants

In certain embodiments, amino acid modifications may be introduced into the Fc region of an antibody provided herein to generate an Fc region variant. In certain embodiments, the Fc region variant possesses some, but not all, effector functions. Such antibodies may be useful, for example, in applications in which the half-life of the antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

In some embodiments, the Fc comprises one or more modifications in at least one of the CH₃ sequences. In some embodiments, the Fc comprises one or more modifications in at least one of the CH₂ sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides. Exemplary modifications in the Fc region are described, for example, in International Patent Application No. PCT/US2017/037545, filed Jun. 14, 2017.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 11. Modified Amino Acids

When the antibody conjugate comprises a modified amino acid, the modified amino acid can be any modified amino acid deemed suitable by the practitioner. In particular embodiments, the modified amino acid comprises a reactive group useful for forming a covalent bond to a linker precursor or to a payload precursor. In certain embodiments, the modified amino acid is a non-natural amino acid. In certain embodiments, the reactive group is selected from the group consisting of amino, carboxy, acetyl, hydrazino, hydrazido, semicarbazido, sulfanyl, azido and alkynyl. Modified amino acids are also described in, for example, WO 2013/185115 and WO 2015/006555, each of which is incorporated herein by reference in its entirety.

In certain embodiments, the amino acid residue is according to any of the following formulas:

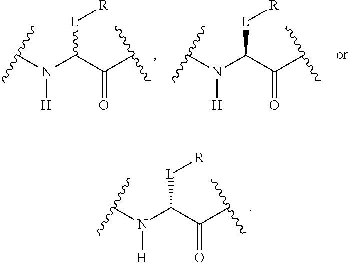

Those of skill in the art will recognize that antibodies are generally comprised of L-amino acids However, with non-natural amino acids, the present methods and compositions provide the practitioner with the ability to use L-, D- or racemic non-natural amino acids at the site-specific positions. In certain embodiments, the non-natural amino acids described herein include D-versions of the natural amino acids and racemic versions of the natural amino acids.

In the above formulas, the wavy lines indicate bonds that connect to the remainder of the polypeptide chains of the antibodies. These non-natural amino acids can be incorporated into polypeptide chains just as natural amino acids are incorporated into the same polypeptide chains. In certain embodiments, the non-natural amino acids are incorporated into the polypeptide chain via amide bonds as indicated in the formulas.

In the above formulas, R designates any functional group without limitation, so long as the amino acid residue is not identical to a natural amino acid residue. In certain embodiments, R can be a hydrophobic group, a hydrophilic group, a polar group, an acidic group, a basic group, a chelating group, a reactive group, a therapeutic moiety or a labeling moiety. In certain embodiments, R is selected from the group consisting of R^{1a}NR^{2a}R^{3a}, R^{1a}C(═O)R^{2a}, R^{1a}C(═O)OR^{2a}, R^{1a}N₃, R^{1a}C(≡CH). In these embodiments, R^{1a} is selected from the group consisting of a bond, alkylene, heteroalkylene, arylene, heteroarylene. R^{2a} and R^{3a} are each independently selected from the group consisting of hydrogen, alkyl and heteroalkyl.

In some embodiments, the non-naturally encoded amino acids include side chain functional groups that react efficiently and selectively with functional groups not found in the 20 common amino acids (including but not limited to, azido, ketone, aldehyde and aminooxy groups) to form stable conjugates. For example, antigen-binding polypeptide that includes a non-naturally encoded amino acid containing an azido functional group can be reacted with a polymer (including but not limited to, poly(ethylene glycol) or, alternatively, a second polypeptide containing an alkyne moiety to form a stable conjugate resulting for the selective reaction of the azide and the alkyne functional groups to form a Huisgen [3+2] cycloaddition product.

Exemplary non-naturally encoded amino acids that may be suitable for use in the present invention and that are useful for reactions with water soluble polymers include, but are not limited to, those with carbonyl, aminooxy, hydrazine, hydrazide, semicarbazide, azide and alkyne reactive groups. In some embodiments, non-naturally encoded amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature-including but not limited to, an alkene, an oxime, a thioether, an amide and the like. Examples of such amino acids also include saccharides that are not commonly found in naturally-occurring proteins such as 2-deoxy-glucose, 2-deoxygalactose and the like.

Many of the non-naturally encoded amino acids provided herein are commercially available, e.g., from Sigma-Aldrich (St. Louis, Mo., USA), Novabiochem (a division of EMD Biosciences, Darmstadt, Germany), or Peptech (Burlington, Mass., USA). Those that are not commercially available are optionally synthesized as provided herein or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). See, also, U.S. Patent Application Publications 2003/0082575 and 2003/0108885, which is incorporated by reference herein. In addition to unnatural amino acids that contain unnatural side chains, unnatural amino acids that may be suitable for use in the present invention also optionally comprise modified backbone structures, including but not limited to, as illustrated by the structures of Formula II-1 and III-1:

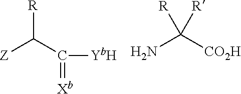

wherein Z typically comprises OH, NH₂, SH, NH—R′, or S—R′; X^{b} and Y^{b}, which can be the same or different, typically comprise S or O, and R and R′, which are optionally the same or different, are typically selected from the same list of constituents for the R group described herein for the unnatural amino acids having Formula I-I as well as hydrogen. For example, unnatural amino acids of the invention optionally comprise substitutions in the amino or carboxyl group as illustrated by Formulas II-I and III-I. Unnatural amino acids of this type include, but are not limited to, α-hydroxy acids, α-thioacids, α-aminothiocarboxylates, including but not limited to, with side chains corresponding to the common twenty natural amino acids or unnatural side chains. In addition, substitutions at the α-carbon optionally include, but are not limited to, L, D, or α-a-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, P and y amino acids such as substituted β-alanine and γ-amino butyric acid.

Many unnatural amino acids are based on natural amino acids, such as tyrosine, glutamine, phenylalanine, and the like, and are suitable for use in the present invention. Tyrosine analogs include, but are not limited to, para-substituted tyrosines, ortho-substituted tyrosines, and meta substituted tyrosines, where the substituted tyrosine comprises, including but not limited to, a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, an alkynyl group or the like. In addition, multiply substituted aryl rings are also contemplated. Glutamine analogs that may be suitable for use in the present invention include, but are not limited to, α-hydroxy derivatives, γ-substituted derivatives, cyclic derivatives, and amide substituted glutamine derivatives. Example phenylalanine analogs that may be suitable for use in the present invention include, but are not limited to, para-substituted phenylalanines, ortho-substituted phenyalanines, and meta-substituted phenylalanines, where the substituent comprises, including but not limited to, a hydroxy group, a methoxy group, a methyl group, an allyl group, an aldehyde, an azido, an iodo, a bromo, a keto group (including but not limited to, an acetyl group), a benzoyl, an alkynyl group, or the like. Specific examples of unnatural amino acids that may be suitable for use in the present invention include, but are not limited to, a p-acetyl-L-phenylalanine, an O-methyl-L-tyrosine, an L-3-(2-naphthyl) alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-azido-methyl-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like. Examples of structures of a variety of unnatural amino acids that may be suitable for use in the present invention are provided in, for example, WO 2002/085923 entitled “In vivo incorporation of unnatural amino acids.” See also Kiick et al., (2002) Incorporation of azides into recombinant proteins for chemoselective modification by the Staudinger ligation, PNAS 99:19-24, for additional methionine analogs.

Many of the unnatural amino acids suitable for use in the present invention are commercially available, e.g., from Sigma (USA) or Aldrich (Milwaukee, Wis., USA). Those that are not commercially available are optionally synthesized as provided herein or as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) A New Synthesis of Glutamine and of y-Dipeptides of Glutamic Acid from Phthylated Intermediates. J. Chem. Soc., 3315-3319; Friedman, O. M. & Chatterrji, R. (1959) Synthesis of Derivatives of Glutamine as Model Substrates for Anti-Tumor Agents. J. Am. Chem. Soc. 81, 3750-3752; Craig, J. C. et al. (1988) Absolute Configuration of the Enantiomers of 7-Chloro-4 [[4-(diethylamino)-1-methylbutyl]amino] quinoline (Chloroquine). J. Org. Chem. 53, 1167-1170; Azoulay, M., Vilmont, M. & Frappier, F. (1991) Glutamine analogues as Potential Antimalarials, Eur. J. Med. Chem. 26, 201-5; Koskinen, A. M. P. & Rapoport, H. (1989) Synthesis of 4-Substituted Prolines as Conformationally Constrained Amino Acid Analogues. J. Org. Chem. 54, 1859-1866; Christie, B. D. & Rapoport, H. (1985) Synthesis of Optically Pure Pipecolates from L-Asparagine. Application to the Total Synthesis of (+)-Apovincamine through Amino Acid Decarbonylation and Iminium Ion Cyclization. J. Org. Chem. 1989:1859-1866; Barton et al., (1987) Synthesis of Novel a-Amino-Acids and Derivatives Using Radical Chemistry: Synthesis of L- and D-a-Amino-Adipic Acids, L-a-aminopimelic Acid and Appropriate Unsaturated Derivatives. Tetrahedron Lett. 43:4297-4308; and, Subasinghe et al., (1992) Quisqualic acid analogues: synthesis of beta-heterocyclic 2-aminopropanoic acid derivatives and their activity at a novel quisqualate-sensitized site. J. Med. Chem. 35:4602-7. See also, patent applications entitled “Protein Arrays,” filed Dec. 22, 2003, Ser. No. 10/744,899 and Ser. No. 60/435,821 filed on Dec. 22, 2002.

Amino acids with a carbonyl reactive group allow for a variety of reactions to link molecules (including but not limited to, PEG or other water soluble molecules) via nucleophilic addition or aldol condensation reactions among others.

Exemplary carbonyl-containing amino acids can be represented as follows:

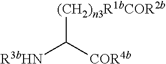

wherein n3 is 0-10; R^{1b} is an alkyl, aryl, substituted alkyl, or substituted aryl; R^{2b} is H, alkyl, aryl, substituted alkyl, and substituted aryl; and R^{3b} is H, an amino acid, a polypeptide, or an amino terminus modification group, and R^{4b} is H, an amino acid, a polypeptide, or a carboxy terminus modification group. In some embodiments, n3 is 1, R^{1b} is phenyl and R^{2b} is a simple alkyl (i.e., methyl, ethyl, or propyl) and the ketone moiety is positioned in the para position relative to the alkyl side chain. In some embodiments, n3 is 1, R^{1b} is phenyl and R^{2b} is a simple alkyl (i.e., methyl, ethyl, or propyl) and the ketone moiety is positioned in the meta position relative to the alkyl side chain.

In some examples, a non-naturally encoded amino acid bearing adjacent hydroxyl and amino groups can be incorporated into the polypeptide as a “masked” aldehyde functionality. For example, 5-hydroxylysine bears a hydroxyl group adjacent to the epsilon amine. Reaction conditions for generating the aldehyde typically involve addition of molar excess of sodium metaperiodate under mild conditions to avoid oxidation at other sites within the polypeptide. The pH of the oxidation reaction is typically about 7.0. A typical reaction involves the addition of about 1.5 molar excess of sodium meta periodate to a buffered solution of the polypeptide, followed by incubation for about 10 minutes in the dark. See, e.g. U.S. Pat. No. 6,423,685, which is incorporated by reference herein.

The carbonyl functionality can be reacted selectively with a hydrazine-, hydrazide-, hydroxylamine-, or semicarbazide-containing reagent under mild conditions in aqueous solution to form the corresponding hydrazone, oxime, or semicarbazone linkages, respectively, that are stable under physiological conditions. See, e.g., Jencks, W. P., J. Am. Chem. Soc. 81, 475-481 (1959); Shao, J. and Tam, J. P., J. Am. Chem. Soc. 117:3893-3899 (1995). Moreover, the unique reactivity of the carbonyl group allows for selective modification in the presence of the other amino acid side chains. See, e.g., Cornish, V. W., et al., J. Am. Chem. Soc. 118:8150-8151 (1996); Geoghegan, K. F. & Stroh, J. G., Bioconjug. Chem. 3:138-146 (1992); Mahal, L. K., et al., Science 276:1125-1128 (1997).

Non-naturally encoded amino acids containing a nucleophilic group, such as a hydrazine, hydrazide or semicarbazide, allow for reaction with a variety of electrophilic groups to form conjugates (including but not limited to, with PEG or other water soluble polymers).

Exemplary hydrazine, hydrazide or semicarbazide-containing amino acids can be represented as follows:

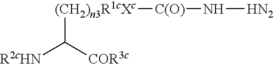

wherein n3 is 0-10; R^{1c} is an alkyl, aryl, substituted alkyl, or substituted aryl or not present; X^{c}, is O, N, or S or not present; R^{2c} is H, an amino acid, a polypeptide, or an amino terminus modification group, and R^{3c} is H, an amino acid, a polypeptide, or a carboxy terminus modification group.

In some embodiments, n3 is 4, R^{1c} is not present, and X is N. In some embodiments, n3 is 2, R^{1c} is not present, and X is not present. In some embodiments, n3 is 1, R^{1c} is phenyl, X is O, and the oxygen atom is positioned para to the aliphatic group on the aryl ring.

Hydrazide-, hydrazine-, and semicarbazide-containing amino acids are available from commercial sources. For instance, L-glutamate-γ-hydrazide is available from Sigma Chemical (St. Louis, Mo.). Other amino acids not available commercially can be prepared by one skilled in the art. See, e.g., U.S. Pat. No. 6,281,211, which is incorporated by reference herein.

Polypeptides containing non-naturally encoded amino acids that bear hydrazide, hydrazine or semicarbazide functionalities can be reacted efficiently and selectively with a variety of molecules that contain aldehydes or other functional groups with similar chemical reactivity. See, e.g., Shao, J. and Tam, J., J. Am. Chem. Soc. 117:3893-3899 (1995). The unique reactivity of hydrazide, hydrazine and semicarbazide functional groups makes them significantly more reactive toward aldehydes, ketones and other electrophilic groups as compared to the nucleophilic groups present on the 20 common amino acids (including but not limited to, the hydroxyl group of serine or threonine or the amino groups of lysine and the N-terminus).

Non-naturally encoded amino acids containing an aminooxy (also called a hydroxylamine) group allow for reaction with a variety of electrophilic groups to form conjugates (including but not limited to, with PEG or other water soluble polymers). Like hydrazines, hydrazides and semicarbazides, the enhanced nucleophilicity of the aminooxy group permits it to react efficiently and selectively with a variety of molecules that contain aldehydes or other functional groups with similar chemical reactivity. See, e.g., Shao, J. and Tam, J., J. Am. Chem. Soc. 117:3893-3899 (1995); H. Hang and C. Bertozzi, Acc. Chem. Res. 34:727-736 (2001). Whereas the result of reaction with a hydrazine group is the corresponding hydrazone, however, an oxime results generally from the reaction of an aminooxy group with a carbonyl-containing group such as a ketone.

Exemplary amino acids containing aminooxy groups can be represented as follows:

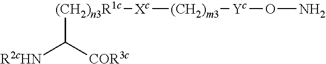

wherein n3 is 0-10; R^{1c} is an alkyl, aryl, substituted alkyl, or substituted aryl or not present; X^{c} is O, N, S or not present; m3 is 0-10; Y^{c} is ═C(O) or not present; R^{2c} is H, an amino acid, a polypeptide, or an amino terminus modification group, and R^{3c} is H, an amino acid, a polypeptide, or a carboxy terminus modification group. In some embodiments, n3 is 1, R^{1a} is phenyl, X^{c} is O, m is 1, and Y^{c} is present. In some embodiments, n3 is 2, R^{1c} and X^{c} are not present, m3 is 0, and Ye is not present.

Aminooxy-containing amino acids can be prepared from readily available amino acid precursors (homoserine, serine and threonine). See, e.g., M. Carrasco and R. Brown, J. Org. Chem. 68:8853-8858 (2003). Certain aminooxy-containing amino acids, such as L-2-amino-4-(aminooxy) butyric acid), have been isolated from natural sources (Rosenthal, G. et al., Life Sci. 60:1635-1641 (1997). Other aminooxy-containing amino acids can be prepared by one skilled in the art.

The unique reactivity of azide and alkyne functional groups makes them extremely useful for the selective modification of polypeptides and other biological molecules. Organic azides, particularly aliphatic azides, and alkynes are generally stable toward common reactive chemical conditions. In particular, both the azide and the alkyne functional groups are inert toward the side chains (i.e., R groups) of the 20 common amino acids found in naturally-occurring polypeptides. When brought into close proximity, however, the “spring-loaded” nature of the azide and alkyne groups is revealed and they react selectively and efficiently via Huisgen [3+2] cycloaddition reaction to generate the corresponding triazole. See, e.g., Chin J., et al., Science 301:964-7 (2003); Wang, Q., et al., J. Am. Chem. Soc. 125, 3192-3193 (2003); Chin, J. W., et al., J. Am. Chem. Soc. 124:9026-9027 (2002).

Because the Huisgen cycloaddition reaction involves a selective cycloaddition reaction (see, e.g., Padwa, A., in COMPREHENSIVE ORGANIC SYNTHESIS, Vol. 4, (ed. Trost, B. M., 1991), p. 1069-1109; Huisgen, R. in 1,3-DIPOLAR CYCLOADDITION CHEMISTRY, (ed. Padwa, A., 1984), p. 1-176) rather than a nucleophilic substitution, the incorporation of non-naturally encoded amino acids bearing azide and alkyne-containing side chains permits the resultant polypeptides to be modified selectively at the position of the non-naturally encoded amino acid. Cycloaddition reaction involving azide or alkyne-containing antibody can be carried out at room temperature under aqueous conditions by the addition of Cu(II) (including but not limited to, in the form of a catalytic amount of CuSO₄) in the presence of a reducing agent for reducing Cu(II) to Cu(I), in situ, in catalytic amount. See, e.g., Wang, Q., et al., J. Am. Chem. Soc. 125, 3192-3193 (2003); Tornoe, C. W., et al., J. Org. Chem. 67:3057-3064 (2002); Rostovtsev, et al., Angew. Chem. Int. Ed. 41:2596-2599 (2002). Exemplary reducing agents include, including but not limited to, ascorbate, metallic copper, quinine, hydroquinone, vitamin K, glutathione, cysteine, Fe²⁺, Co²⁺, and an applied electric potential.

In some cases, where a Huisgen [3+2] cycloaddition reaction between an azide and an alkyne is desired, the antigen-binding polypeptide comprises a non-naturally encoded amino acid comprising an alkyne moiety and the water soluble polymer to be attached to the amino acid comprises an azide moiety. Alternatively, the converse reaction (i.e., with the azide moiety on the amino acid and the alkyne moiety present on the water soluble polymer) can also be performed.

The azide functional group can also be reacted selectively with a water soluble polymer containing an aryl ester and appropriately functionalized with an aryl phosphine moiety to generate an amide linkage. The aryl phosphine group reduces the azide in situ and the resulting amine then reacts efficiently with a proximal ester linkage to generate the corresponding amide. See, e.g., E. Saxon and C. Bertozzi, Science 287, 2007-2010 (2000). The azide-containing amino acid can be either an alkyl azide (including but not limited to, 2-amino-6-azido-1-hexanoic acid) or an aryl azide (p-azido-phenylalanine).

Exemplary water soluble polymers containing an aryl ester and a phosphine moiety can be represented as follows:

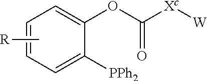

wherein X^{c} can be O, N, S or not present, Ph is phenyl, W is a water soluble polymer and R can be H, alkyl, aryl, substituted alkyl and substituted aryl groups. Exemplary R groups include but are not limited to —CH₂, —C(CH₃)₃, OR′, —NR′R″, —SR′, -halogen, —C(O) R′, —CONR′R″, —S(O)₂R′, —S(O)₂NR′R″, —CN and —NO₂. R′, R″, R′″ and R″″ each independently refer to hydrogen, substituted or unsubstituted heteroalkyl, substituted or unsubstituted aryl, including but not limited to, aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (including but not limited to, —CF₃ and —CH₂CF₃) and acyl (including but not limited to, —C(O)CH₃, C(O)CF₃, —C(O)CH₂OCH₃, and the like).

The azide functional group can also be reacted selectively with a water soluble polymer containing a thioester and appropriately functionalized with an aryl phosphine moiety to generate an amide linkage. The aryl phosphine group reduces the azide in situ and the resulting amine then reacts efficiently with the thioester linkage to generate the corresponding amide. Exemplary water soluble polymers containing a thioester and a phosphine moiety can be represented as follows:

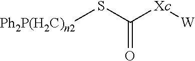

wherein n2 is 1-10; Xc can be O, N, S or not present, Ph is phenyl, and W is a water soluble polymer.

Exemplary alkyne-containing amino acids can be represented as follows:

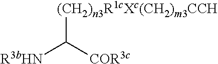

wherein n3 is 0-10; R^{1c} is an alkyl, aryl, substituted alkyl, or substituted aryl or not present; X^{c} is O, N, S or not present; m3 is 0-10, R^{2c} is H, an amino acid, a polypeptide, or an amino terminus modification group, and R^{3c} is H, an amino acid, a polypeptide, or a carboxy terminus modification group. In some embodiments, n3 is 1, R^{1c} is phenyl, X^{c} is not present, m3 is 0 and the acetylene moiety is positioned in the para position relative to the alkyl side chain. In some embodiments, n3 is 1, R^{1c} is phenyl, X^{c} is O, m3 is 1 and the propargyloxy group is positioned in the para position relative to the alkyl side chain (i.e., O-propargyl-tyrosine). In some embodiments, n3 is 1, R^{1c} and X^{c} are not present and m3 is 0 (i.e., proparylglycine).

Alkyne-containing amino acids are commercially available. For example, propargylglycine is commercially available from Peptech (Burlington, Mass.). Alternatively, alkyne-containing amino acids can be prepared according to standard methods. For instance, p-propargyloxyphenylalanine can be synthesized, for example, as described in Deiters, A., et al., J. Am. Chem. Soc. 125:11782-11783 (2003), and 4-alkynyl-L-phenylalanine can be synthesized as described in Kayser, B., et al., Tetrahedron 53 (7): 2475-2484 (1997). Other alkyne-containing amino acids can be prepared by one skilled in the art.

Exemplary azide-containing amino acids can be represented as follows:

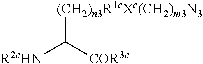

wherein n3 is 0-10; R^{1c} is an alkyl, aryl, substituted alkyl, substituted aryl or not present; X^{c} is O, N, S or not present; m3 is 0-10; R^{2c} is H, an amino acid, a polypeptide, or an amino terminus modification group, and R^{3c} is H, an amino acid, a polypeptide, or a carboxy terminus modification group. In some embodiments, n3 is 1, R^{1c} is phenyl, X^{c} is not present, m3 is 0 and the azide moiety is positioned para to the alkyl side chain. In some embodiments, n3 is 0-4 and R^{1c} and X^{c} are not present, and m3=0. In some embodiments, n3 is 1, R^{1c} is phenyl, X^{c} is O, m3 is 2 and the P-azidoethoxy moiety is positioned in the para position relative to the alkyl side chain.

Azide-containing amino acids are available from commercial sources. For instance, 4-azidophenylalanine can be obtained from Chem-Impex International, Inc. (Wood Dale, Ill.). For those azide-containing amino acids that are not commercially available, the azide group can be prepared relatively readily using standard methods known to those of skill in the art, including but not limited to, via displacement of a suitable leaving group (including but not limited to, halide, mesylate, tosylate) or via opening of a suitably protected lactone. See, e.g., Advanced Organic Chemistry by March (Third Edition, 1985, Wiley and Sons, New York).

The unique reactivity of beta-substituted aminothiol functional groups makes them extremely useful for the selective modification of polypeptides and other biological molecules that contain aldehyde groups via formation of the thiazolidine. See, e.g., J. Shao and J. Tam, J. Am. Chem. Soc. 1995, 117 (14) 3893-3899. In some embodiments, beta-substituted aminothiol amino acids can be incorporated into antibodies and then reacted with water soluble polymers comprising an aldehyde functionality. In some embodiments, a water soluble polymer, drug conjugate or other payload can be coupled to an antibody polypeptide comprising a beta-substituted aminothiol amino acid via formation of the thiazolidine.

Particular examples of useful non-natural amino acids include, but are not limited to, p-acetyl-L-phenylalanine, O-methyl-L-tyrosine, L-3-(2-naphthyl) alanine, 3-methyl-phenylalanine, O-4-allyl-L-tyrosine, 4-propyl-L-tyrosine, tri-O-acetyl-GlcNAc b-serine, L-Dopa, fluorinated phenylalanine, isopropyl-L-phenylalanine, p-azido-methyl-L-phenylalanine, p-azido-L-phenylalanine, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine. Further useful examples include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine.

In particular embodiments, the non-natural amino acids are selected from p-acetyl-phenylalanine, p-ethynyl-phenylalanine, p-propargyloxyphenylalanine, p-azido-methyl-phenylalanine, and p-azido-phenylalanine. One particularly useful non-natural amino acid is p-azido phenylalanine. This amino acid residue is known to those of skill in the art to facilitate Huisgen [3+2] cyloaddition reactions (so-called “click” chemistry reactions) with, for example, compounds bearing alkynyl groups. This reaction enables one of skill in the art to readily and rapidly conjugate to the antibody at the site-specific location of the non-natural amino acid.

In certain embodiments, the first reactive group is an alkynyl moiety (including but not limited to, in the unnatural amino acid p-propargyloxyphenylalanine, where the propargyl group is also sometimes referred to as an acetylene moiety) and the second reactive group is an azido moiety, and [3+2] cycloaddition chemistry can be used. In certain embodiments, the first reactive group is the azido moiety (including but not limited to, in the unnatural amino acid p-azido-L-phenylalanine) and the second reactive group is the alkynyl moiety.

In the above formulas, each L represents a divalent linker. The divalent linker can be any divalent linker known to those of skill in the art. Generally, the divalent linker is capable of forming covalent bonds to the functional moiety R and the cognate reactive group (e.g., alpha carbon) of the non-natural amino acid. Useful divalent linkers a bond, alkylene, substituted alkylene, heteroalkylene, substituted heteroalkylene, arylene, substituted arylene, heteroarlyene and substituted heteroarylene. In certain embodiments, L is C₁₋₁₀ alkylene or C₁₋₁₀ heteroalkylene.

The non-natural amino acids used in the methods and compositions described herein have at least one of the following four properties: (1) at least one functional group on the sidechain of the non-natural amino acid has at least one characteristics and/or activity and/or reactivity orthogonal to the chemical reactivity of the 20 common, genetically-encoded amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), or at least orthogonal to the chemical reactivity of the naturally occurring amino acids present in the polypeptide that includes the non-natural amino acid; (2) the introduced non-natural amino acids are substantially chemically inert toward the 20 common, genetically-encoded amino acids; (3) the non-natural amino acid can be stably incorporated into a polypeptide, preferably with the stability commensurate with the naturally-occurring amino acids or under typical physiological conditions, and further preferably such incorporation can occur via an in vivo system; and (4) the non-natural amino acid includes an oxime functional group or a functional group that can be transformed into an oxime group by reacting with a reagent, preferably under conditions that do not destroy the biological properties of the polypeptide that includes the non-natural amino acid (unless of course such a destruction of biological properties is the purpose of the modification/transformation), or where the transformation can occur under aqueous conditions at a pH between about 4 and about 8, or where the reactive site on the non-natural amino acid is an electrophilic site. Any number of non-natural amino acids can be introduced into the polypeptide. Non-natural amino acids may also include protected or masked oximes or protected or masked groups that can be transformed into an oxime group after deprotection of the protected group or unmasking of the masked group. Non-natural amino acids may also include protected or masked carbonyl or dicarbonyl groups, which can be transformed into a carbonyl or dicarbonyl group after deprotection of the protected group or unmasking of the masked group and thereby are available to react with hydroxylamines or oximes to form oxime groups.

In further embodiments, non-natural amino acids that may be used in the methods and compositions described herein include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or non-covalently interact with other molecules, photocaged and/or photoisomerizable amino acids, amino acids comprising biotin or a biotin analogue, glycosylated amino acids such as a sugar substituted serine, other carbohydrate modified amino acids, keto-containing amino acids, aldehyde-containing amino acids, amino acids comprising polyethylene glycol or other polyethers, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

In some embodiments, non-natural amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature-including but not limited to, an alkene, an oxime, a thioether, an amide and the like. Examples of such amino acids also include saccharides that are not commonly found in naturally-occurring proteins such as 2-deoxy-glucose, 2-deoxygalactose and the like.

The chemical moieties incorporated into antibodies via incorporation of non-natural amino acids offer a variety of advantages and manipulations of polypeptides. For example, the unique reactivity of a carbonyl or dicarbonyl functional group (including a keto- or aldehyde-functional group) allows selective modification of antibodies with any of a number of hydrazine- or hydroxylamine-containing reagents in vivo and in vitro. A heavy atom non-natural amino acid, for example, can be useful for phasing x-ray structure data. The site-specific introduction of heavy atoms using non-natural amino acids also provides selectivity and flexibility in choosing positions for heavy atoms. Photoreactive non-natural amino acids (including but not limited to, amino acids with benzophenone and arylazides (including but not limited to, phenylazide) side chains), for example, allow for efficient in vivo and in vitro photocrosslinking of polypeptides. Examples of photoreactive non-natural amino acids include, but are not limited to, p-azido-phenylalanine and p-benzoyl-phenylalanine. The antibodies with the photoreactive non-natural amino acids may then be crosslinked at will by excitation of the photoreactive group-providing temporal control. In a non-limiting example, the methyl group of a non-natural amino can be substituted with an isotopically labeled, including but not limited to, with a methyl group, as a probe of local structure and dynamics, including but not limited to, with the use of nuclear magnetic resonance and vibrational spectroscopy.

Amino acids with an electrophilic reactive group allow for a variety of reactions to link molecules via various chemical reactions, including, but not limited to, nucleophilic addition reactions. Such electrophilic reactive groups include a carbonyl- or dicarbonyl-group (including a keto- or aldehyde group), a carbonyl-like- or dicarbonyl-like-group (which has reactivity similar to a carbonyl- or dicarbonyl-group and is structurally similar to a carbonyl- or dicarbonyl-group), a masked carbonyl- or masked dicarbonyl-group (which can be readily converted into a carbonyl- or dicarbonyl-group), or a protected carbonyl- or protected dicarbonyl-group (which has reactivity similar to a carbonyl- or dicarbonyl-group upon deprotection). Such amino acids include amino acids having the structure of Formula (I-1):

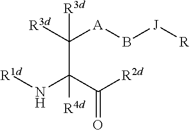

wherein: A is optional, and when present is lower alkylene, substituted lower alkylene, lower cycloalkylene, substituted lower cycloalkylene, lower alkenylene, substituted lower alkenylene, alkynylene, lower heteroalkylene, substituted heteroalkylene, lower heterocycloalkylene, substituted lower heterocycloalkylene, arylene, substituted arylene, heteroarylene, substituted heteroarylene, alkarylene, substituted alkarylene, aralkylene, or substituted aralkylene; B is optional, and when present is a linker selected from the group consisting of lower alkylene, substituted lower alkylene, lower alkenylene, substituted lower alkenylene, lower heteroalkylene, substituted lower heteroalkylene, —O—, —O-(alkylene or substituted alkylene)-,
—S—, —S-(alkylene or substituted alkylene)-, —S(O)ₖ— where k is 1, 2, or 3, —S(O)ₖ (alkylene or substituted alkylene)-, —C(O)—, —NS(O)₂—, —OS(O)₂—, —C(O)-(alkylene or substituted alkylene)-,
—C(S)—, —C(S)-(alkylene or substituted alkylene)-, —N(R′)—, —NR′ (alkylene or substituted alkylene)-, —C(O)N(R′)—, —CON(R′)-(alkylene or substituted alkylene)-, —CSN(R′)—, —CSN(R′)-(alkylene or substituted alkylene)-, —N(R′)CO-(alkylene or substituted alkylene)-,
—N(R′)C(O)O—, —S(O)ₖN(R′)—, —N(R′)C(O)N(R′)—, —N(R′)C(S) N(R′)—,
—N(R′)S(O)ₖN(R′)—, —N(R′)—N═, —C(R′)═N—, —C(R′)═N—N(R′)—, —C(R′)═N—N═, C(R′)₂—N═N—, and —C(R′)₂—N(R′)—N(R′)—, where each R′ is independently H, alkyl, or substituted alkyl; J is

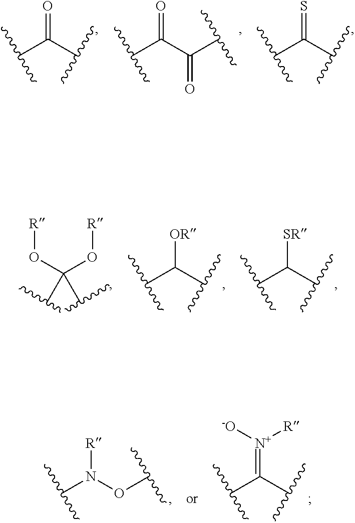

R is H, alkyl, substituted alkyl, cycloalkyl, or substituted cycloalkyl; each R″ is independently H, alkyl, substituted alkyl, or a protecting group, or when more than one R″ group is present, two R″ optionally form a heterocycloalkyl; R^{1d} is H, an amino protecting group, resin, amino acid, polypeptide, or polynucleotide; and R^{2d} is OH, an ester protecting group, resin, amino acid, polypeptide, or polynucleotide; each of R^{3d} and R^{4d} is independently H, halogen, lower alkyl, or substituted lower alkyl, or R^{3d} and R^{4d} or two R^{3d} groups optionally form a cycloalkyl or a heterocycloalkyl; or the -A-B-J-R groups together form a bicyclic or tricyclic cycloalkyl or heterocycloalkyl comprising at least one carbonyl group, including a dicarbonyl group, protected carbonyl group, including a protected dicarbonyl group, or masked carbonyl group, including a masked dicarbonyl group; or the -J-R group together forms a monocyclic or bicyclic cycloalkyl or heterocycloalkyl comprising at least one carbonyl group, including a dicarbonyl group, protected carbonyl group, including a protected dicarbonyl group, or masked carbonyl group, including a masked dicarbonyl group; with a proviso that when A is phenylene and each R^{3d} is H, B is present; and that when A is —(CH₂)₄— and each R^{3d} is H, B is not —NHC(O)(CH₂CH₂)—; and that when A and B are absent and each R^{3d} is H, R is not methyl. Such non-natural amino acids may be in the form of a salt, or may be incorporated into a non-natural amino acid polypeptide, polymer, polysaccharide, or a polynucleotide and optionally post translationally modified.

In certain embodiments, compounds of Formula (I-1) are stable in aqueous solution for at least 1 month under mildly acidic conditions. In certain embodiments, compounds of Formula (I-1) are stable for at least 2 weeks under mildly acidic conditions. In certain embodiments, compound of Formula (I-1) are stable for at least 5 days under mildly acidic conditions. In certain embodiments, such acidic conditions are pH 2 to 8.

In certain embodiments of compounds of Formula (I-1), B is lower alkylene, substituted lower alkylene, —O-(alkylene or substituted alkylene)-, —C(R′)═N—N(R′)—, —N(R′)CO—, —C(O)—, —C(R′)═N—, —C(O)-(alkylene or substituted alkylene)-, —CON(R′)-(alkylene or substituted alkylene)-, —S(alkylene or substituted alkylene)-, —S(O) (alkylene or substituted alkylene)-, or —S(O)₂ (alkylene or substituted alkylene)-. In certain embodiments of compounds of Formula (I-1), B is —O(CH₂)—, —CH═N—, —CH═N—NH—, —NHCH₂—, —NHCO—, —C(O)—, —C(O)—(CH₂)—, —CONH—(CH₂)—, —SCH₂—, —S(═O)CH₂—, or —S(O)₂CH₂—. In certain embodiments of compounds of Formula (I-1), R is C₁₋₆ alkyl or cycloalkyl. In certain embodiments of compounds of Formula (I-1) R is —CH₃, —CH(CH₃)₂, or cyclopropyl. In certain embodiments of compounds of Formula (I-1), R^{1d} is H, tert-butyloxycarbonyl (Boc), 9-Fluorenylmethoxycarbonyl (Fmoc), N-acetyl, tetrafluoroacetyl (TFA), or benzyloxycarbonyl (Cbz). In certain embodiments of compounds of Formula (I-1), Rid is a resin, amino acid, polypeptide, or polynucleotide. In certain embodiments of compounds of Formula (I-1), R^{2d} is OH, O-methyl, O-ethyl, or O-t-butyl. In certain embodiments of compounds of Formula (I-1), R^{2d} is a resin, amino acid, polypeptide, or polynucleotide. In certain embodiments of compounds of Formula (I-1), R^{2d} is a polynucleotide. In certain embodiments of compounds of Formula (I-1), R^{2d} is ribonucleic acid (RNA). In certain embodiments of compounds of Formula (I-1), R^{2d} is tRNA. In certain embodiments of compounds of Formula (I-1), the tRNA specifically recognizes a selector codon. In certain embodiments of compounds of Formula (I-1) the selector codon is selected from the group consisting of an amber codon, ochre codon, opal codon, a unique codon, a rare codon, an unnatural codon, a five-base codon, and a four-base codon. In certain embodiments of compounds of Formula (I-1), R^{2d} is a suppressor tRNA.

In certain embodiments of compounds of Formula (I-2),

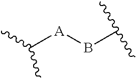

is selected from the group consisting of: (i) A is substituted lower alkylene, C₄-arylene, substituted arylene, heteroarylene, substituted heteroarylene, alkarylene, substituted alkarylene, aralkylene, or substituted aralkylene; B is optional, and when present is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S) N(R′)—, —N(R′)S(O)N(R′)—, —N(R′)S(O)₂N(R′)—, —N(R′)—N═, —C(R′)═N—N(R′)—, —C(R′)═N—N═, —C(R′)₂—N═N—, and —C(R′)₂—N(R′)—N(R′)—; (ii) A is optional, and when present is substituted lower alkylene, C₄-arylene, substituted arylene, heteroarylene, substituted heteroarylene, alkarylene, substituted alkarylene, aralkylene, or substituted aralkylene; B is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S) N(R′)—, —N(R′)S(O)N(R′)—, —N(R′)S(O)₂N(R′)—, —N(R′)—N═, —C(R′)—N—N(R′)—, —C(R′)═N—N═, —C(R′)₂—N═N—, and —C(R′)₂—N(R′)—N(R′)—; (iii) A is lower alkylene; B is optional, and when present is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—,
—CSN(R′)—, —CON(R′)-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S) N(R′)—, —N(R′)S(O)N(R′)—, —N(R′)S(O)₂N(R′)—, —N(R′)—N═, —C(R′)—N—N(R′)—, —C(R′)═N—N═, —C(R′)₂—N═N—, and —C(R′)₂—N(R′)—N(R′)—; and (iv) A is phenylene; B is a divalent linker selected from the group consisting of lower alkylene, substituted lower alkylene, lower alkenylene, substituted lower alkenylene, —O—, —O-(alkylene or substituted alkylene)-, —S—, —S(O)—, —S(O)₂—, —NS(O)₂—, —OS(O)₂—, —C(O)—, —C(O)-(alkylene or substituted alkylene)-, —C(S)—, —N(R′)—, —C(O)N(R′)—, —CON(R′)-(alkylene or substituted alkylene)-, —CSN(R′)—, —N(R′)CO-(alkylene or substituted alkylene)-, —N(R′)C(O)O—, —N(R′)C(S)—, —S(O)N(R′), —S(O)₂N(R′), —N(R′)C(O)N(R′)—, —N(R′)C(S) N(R′)—, —N(R′)S(O)N(R′)—, —N(R′)S(O)₂N(R′)—, —N(R′)—N═, —C(R′)′N—N(R′)—, —C(R′)═N—N═, —C(R′)₂—N═N—, and —C(R′)₂—N(R′)—N(R′)—; J is

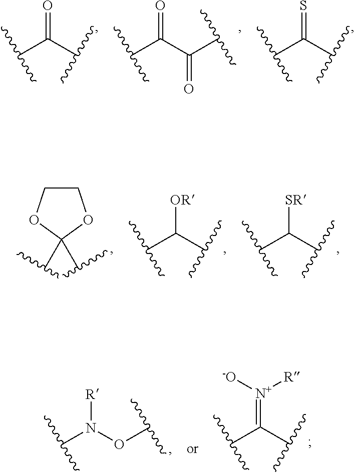

each R′ is independently H, alkyl, or substituted alkyl; R^{1d} is optional, and when present, is H, an amino protecting group, resin, amino acid, polypeptide, or polynucleotide; and R^{2d} is optional, and when present, is OH, an ester protecting group, resin, amino acid, polypeptide, or polynucleotide; and each R^{3d} and R^{4d} is independently H, halogen, lower alkyl, or substituted lower alkyl; and R is H, alkyl, substituted alkyl, cycloalkyl, or substituted cycloalkyl.

In certain embodiments, the non-natural amino acid can be according to formula XIX:

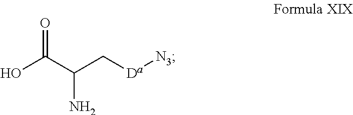

or a salt thereof, wherein: D^{a} is —Ar-W^{3a}- or —W^{1a}—Y^{1a}—C(O)—Y^{2a}—W^{2a}—; Ar is

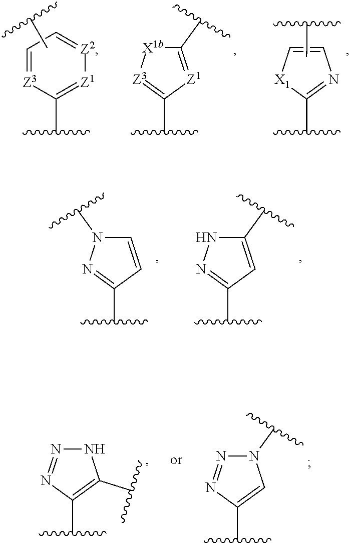

each of W^{1a}, W^{2a}, and W^{3a} is independently a single bond or lower alkylene; each X^{1b} is independently —NH—, —O—, or —S—; each Y^{1a} is independently a single bond, —NH—, or —O—; each Y^{2a} is independently a single bond, —NH—, —O—, or an N-linked or C-linked pyrrolidinylene; and one of Z¹, Z², and Z³ is —N— and the others of Z¹, Z², and Z³ are independently —CH—. In certain embodiments, the non-natural amino acid is according to formula XIXa:

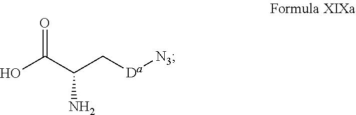

where D^{a} is a defined in the context of formula XIX. In certain embodiments, the non-natural amino acid is according formula XIXb:

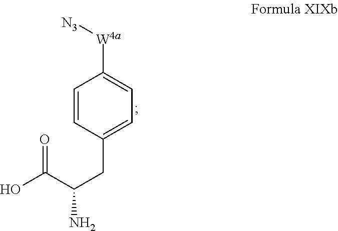

or a salt thereof, wherein W^{4a} is C₁-C₁₀ alkylene. In a further embodiment, W^{4a} is C₁-C₅ alkylene. In an embodiment, W^{4a} is C₁-C₃ alkylene. In an embodiment, W^{4a} is C₁ alkylene. In particular embodiments, the non-natural amino acid is selected from the group consisting of:

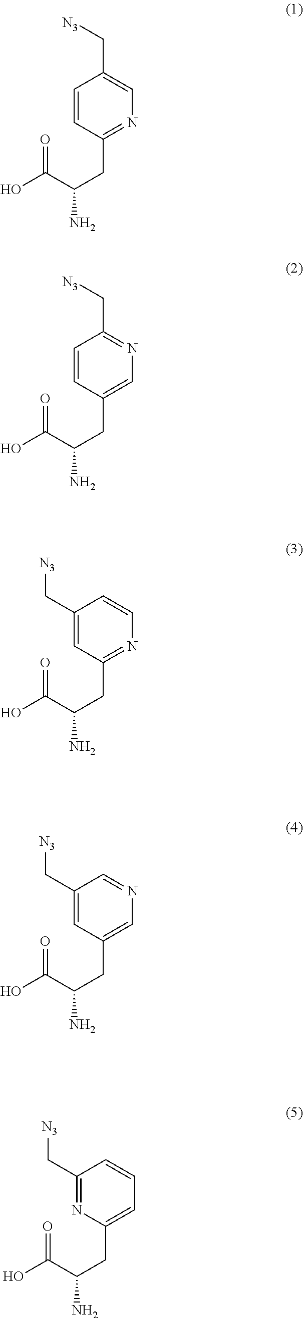 [SMILES: [N-]=[N+]=NCc1ccc(C[C@H](N)C(=O)O)nc1 ; [N-]=[N+]=NCc1ccc(C[C@H](N)C(=O)O)cn1 ; [N-]=[N+]=NCc1ccnc(C[C@H](N)C(=O)O)c1 ; [N-]=[N+]=NCc1cncc(C[C@H](N)C(=O)O)c1 ; [N-]=[N+]=NCc1cccc(C[C@H](N)C(=O)O)n1]

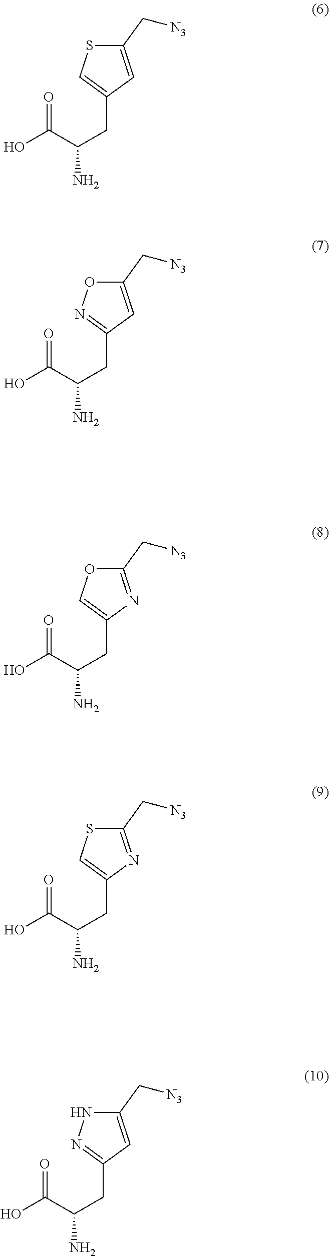 [SMILES: [N-]=[N+]=NCc1cc(C[C@H](N)C(=O)O)cs1 ; [N-]=[N+]=NCc1cc(C[C@H](N)C(=O)O)no1 ; [N-]=[N+]=NCc1nc(C[C@H](N)C(=O)O)co1 ; [N-]=[N+]=NCc1nc(C[C@H](N)C(=O)O)cs1 ; [N-]=[N+]=NCc1cc(C[C@H](N)C(=O)O)n[nH]1]

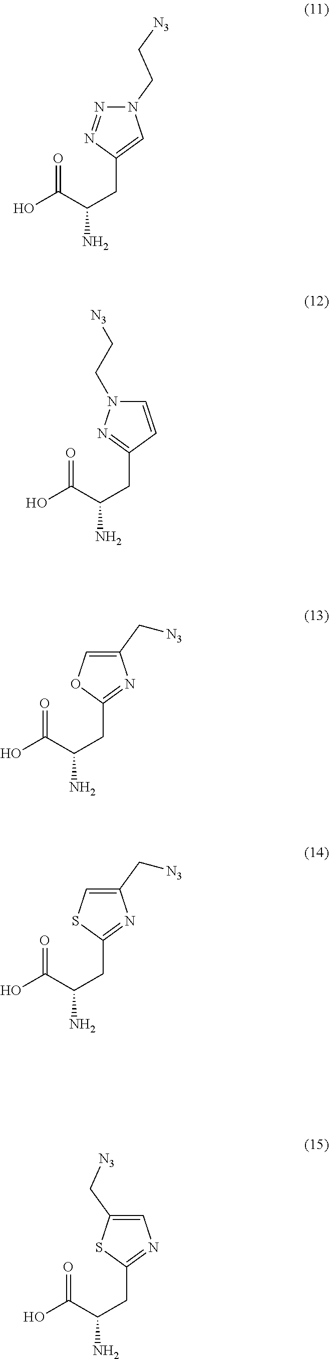 [SMILES: [N-]=[N+]=NCCn1cc(C[C@H](N)C(=O)O)nn1 ; [N-]=[N+]=NCCn1ccc(C[C@H](N)C(=O)O)n1 ; [N-]=[N+]=NCc1coc(C[C@H](N)C(=O)O)n1 ; [N-]=[N+]=NCc1csc(C[C@H](N)C(=O)O)n1 ; [N-]=[N+]=NCc1cnc(C[C@H](N)C(=O)O)s1]

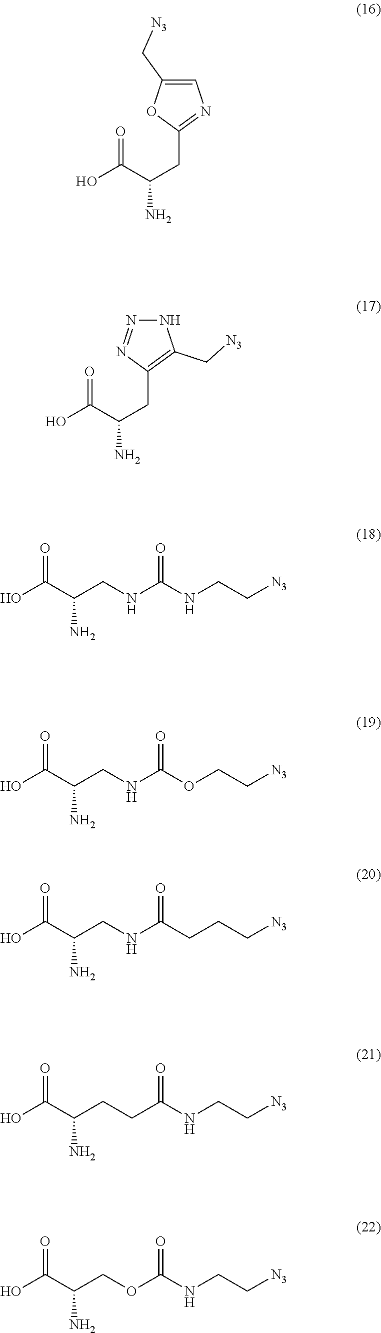

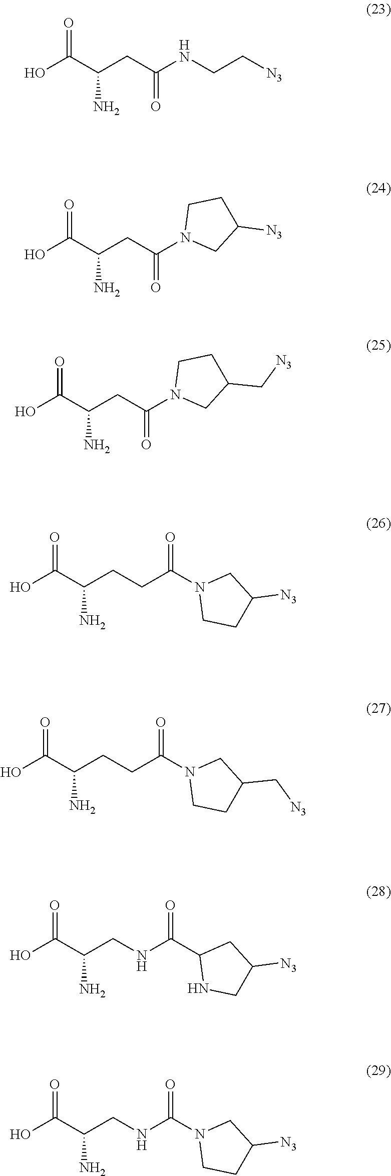

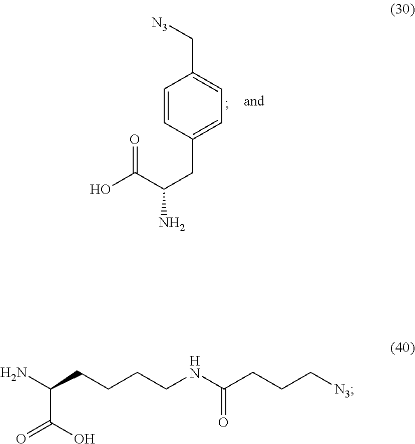 [SMILES: [N-]=[N+]=NCc1ccc(C[C@H](N)C(=O)O)cc1 ; [N-]=[N+]=NCCCC(=O)NCCCC[C@H](N)C(=O)O]

or a salt thereof. Such non-natural amino acids may be in the form of a salt, or may be incorporated into a non-natural amino acid polypeptide, polymer, polysaccharide, or a polynucleotide and optionally post translationally modified.

In certain embodiments, the modified amino acid is according to formula I:

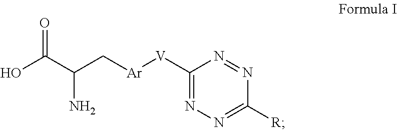

or a salt thereof, wherein Ar is:

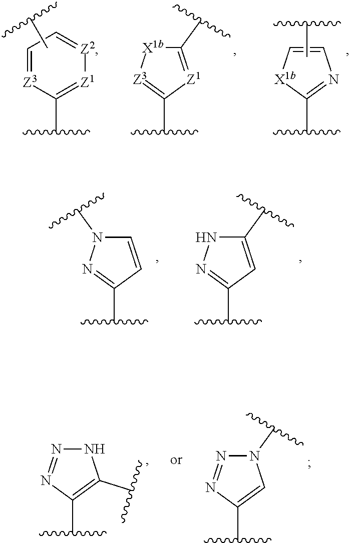

V is a single bond, lower alkylene, or —W^{1a}—W^{2a}-; one of W^{1a} and W^{2a} is absent or lower alkylene, and the other is —NH—, —O—, or —S—; each X^{1b} is independently-NH—, —O—, or —S—; one of Z¹, Z², and Z³ is —CH— or —N— and the others of Z¹, Z², and Z³ are each independently —CH—; and R is lower alkyl. In certain embodiments, when Ar is

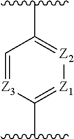

and V is —NH—, then one of Z¹, Z², and Z³ is —N—. In certain embodiments, V is a single bond, —NH—, or —CH₂NH—.

In certain embodiments, Ar is

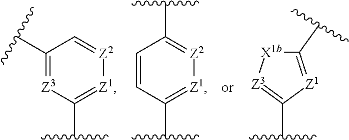

and Z¹, Z², Z³ and X^{1b} are as defined in the context of formula I. In certain embodiments according to this paragraph, V is —W^{1a}—W^{2a}-; one of W^{1a} and W^{2a} is absent or —CH₂—, and the other is —NH—, —O—, or —S—. In certain embodiments according to this paragraph, V is a single bond, —NH—, or —CH₂NH—. In certain embodiments according to this paragraph, Z¹ is N. In certain embodiments according to this paragraph, Z² is N. In certain embodiments according to this paragraph, Z³ is N. In certain embodiments according to this paragraph, Z¹ is CH, Z³ is CH and X^{1b} is S.

In certain embodiments, Ar is

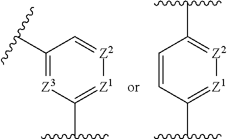

and Z¹, Z², and Z³ are as defined in the context of formula I. In certain embodiments according to this paragraph, V is —W^{1a}—W^{2a}-; one of W^{1a} and W^{2a} is absent or —CH₂—, and the other is —NH—, —O—, or —S—. In certain embodiments according to this paragraph, V is a single bond, —NH—, or —CH₂NH—. In certain embodiments according to this paragraph, Z¹ is N. In certain embodiments according to this paragraph, Z² is N. In certain embodiments according to this paragraph, Z³ is N.

In certain embodiments, Ar is

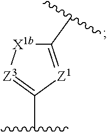

and Z¹, Z³ and X¹ are as defined in the context of Formula I-1. In certain embodiments according to this paragraph, V is —W^{1a}—W^{2a}-; one of W^{1a} and W^{2a} is absent or —CH₂—, and the other is —NH—, —O—, or —S—. In certain embodiments according to this paragraph, V is a single bond, —NH—, or —CH₂NH—. In certain embodiments according to this paragraph, Z¹ is N. In certain embodiments according to this paragraph, Z³ is N. In certain embodiments according to this paragraph, Z¹ is CH, Z³ is CH and X^{1b} is S.

In certain embodiments, the modified amino acid is according to Formula Ia:

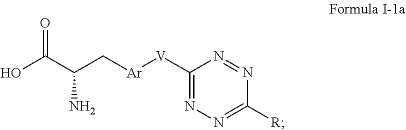

where Ar, V, and R are defined in the context of Formula I-1.

In an embodiment, compounds of either of Formulas I-1 and I-la are provided wherein V is a single bond. In another embodiment, compounds of either of Formulas I-1 and I-1a are provided wherein V is —NH—. In another embodiment, compounds of either of Formulas I-1 and I-1a are provided wherein V is —CH₂NH—.

In certain embodiments, the modified amino acid is according to Formula II-1:

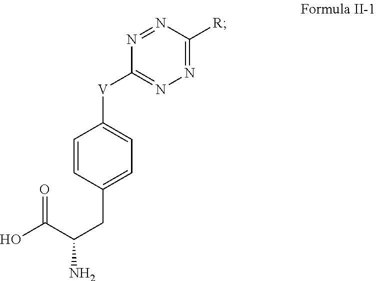

or a salt thereof, wherein V and R are as defined in Formula I-1. In certain embodiments according to this paragraph, V is —W^{1a}—W^{2a}-; one of W^{1a} and W^{2a} is absent or —CH₂—, and the other is —NH—, —O—, or —S—. In certain embodiments, V is a single bond, —NH—, or —CH₂NH—. In certain embodiments, V is a single bond or —CH₂NH—; and R is methyl.

In certain embodiments, the modified amino acid is according to Formula III-1:

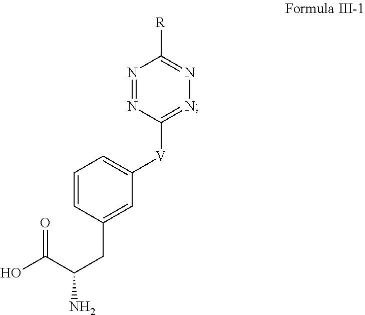

or a salt thereof, wherein V and R are as defined in Formula I-1. In certain embodiments according to this paragraph, V is —W^{1a}—W^{2a}—; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula IV-1:

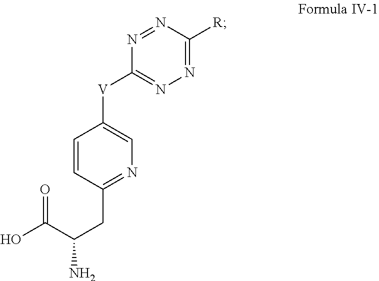

or a salt thereof, wherein V and R are as defined in Formula I-1. In certain embodiments according to this paragraph, V is —W^{1a}—W^{2a}—; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula V-1:

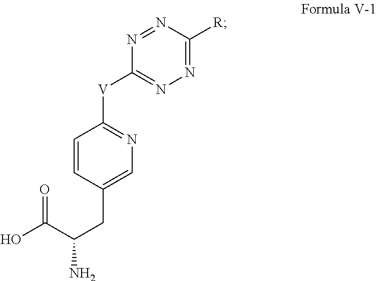

or a salt thereof, wherein V and R are as defined in Formula I. In certain embodiments according to this paragraph, V is —W^{1a}—W^{2a}—; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula VI-1:

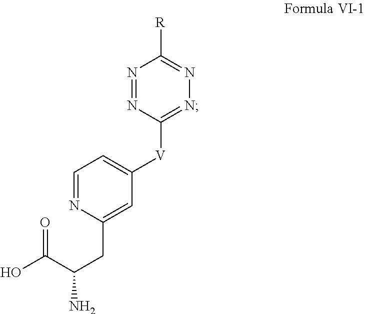

or a salt thereof, wherein V and R are as defined in Formula I. In certain embodiments according to this paragraph, V is —W^{1a}—W^{2a}—; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula VII-1:

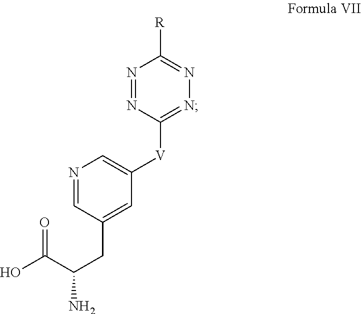

or a salt thereof, wherein V and R are as defined in Formula I. In certain embodiments according to this paragraph, V is —W^{1a}—W^{2a}—; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula VIII-1:

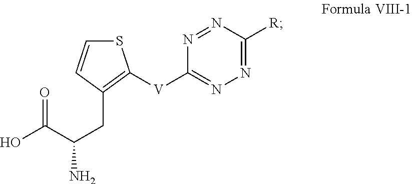

Or a salt thereof, wherein V and R are as defined in Formula I. In certain embodiments according to this paragraph, V is —W^{1a}—W^{2a}—; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to Formula IX-1:

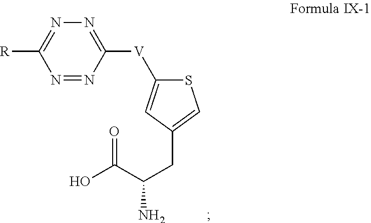

or a salt thereof, wherein V and R are as defined in Formula I-1. In certain embodiments according to this paragraph, V is —W^{1a}—W^{2a}—; one of W^{1a} and W^{2a} is absent or —CH₂—, and the other is —NH—, —O—, or —S—. In certain embodiments, V is a single bond, —NH—, or —CH₂NH—. In certain embodiments, V is a single bond, —NH—, or —CH₂NH—; and R is methyl.

In certain embodiments, the modified amino acid is according to any of formulas 51-62:

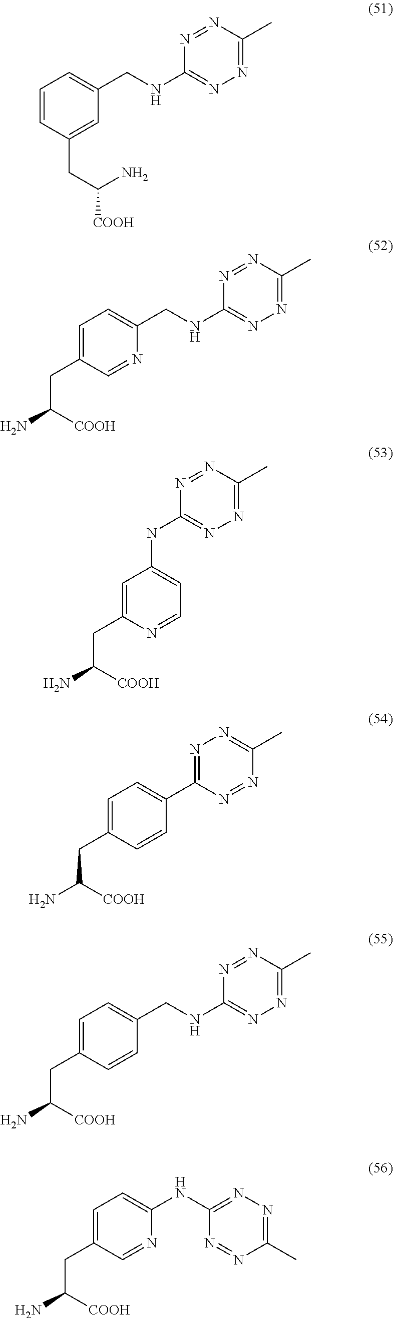

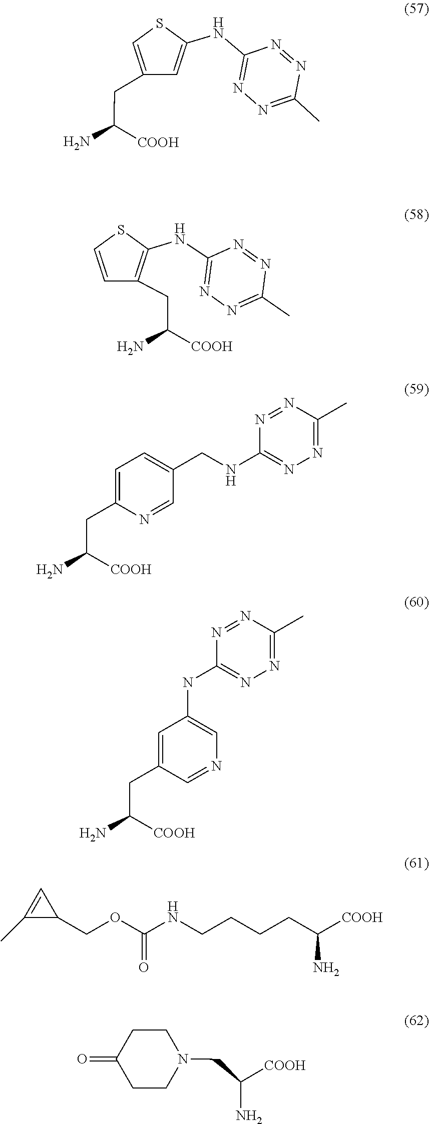

or a salt thereof.

In certain embodiments, the non-natural amino acid is selected from the group consisting of compounds 30, 53, 56, 59, 60, 61, and 62 above. In certain embodiments, the non-natural amino acid is compound 30. In certain embodiments, the non-natural amino acid is compound 56. In some embodiments, the non-natural amino acid is compound 61. In some embodiments, the non-natural amino acid is compound 62.

### 12. Preparation of Antibodies and Antibody Conjugates

### 12.1. Antigen Preparation

The ROR1 protein to be used for isolation of the antibodies may be intact ROR1 or a fragment of ROR1. The intact ROR1 protein, or fragment of ROR1, may be in the form of an isolated protein or protein expressed by a cell. Other forms of ROR1 useful for generating antibodies will be apparent to those skilled in the art.

### 12.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, CA, incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, CA), and SP-2 or X63-Ag8-653 cells (available from the American Type Culture Collection, Rockville, MD). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

### 12.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

### 12.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety).

### 12.5. Conjugation

The antibody conjugates can be prepared by standard techniques. In certain embodiments, an antibody is contacted with a payload precursor under conditions suitable for forming a bond from the antibody to the payload to form an antibody-payload conjugate. In certain embodiments, an antibody is contacted with a linker precursor under conditions suitable for forming a bond from the antibody to the linker. The resulting antibody-linker is contacted with a payload precursor under conditions suitable for forming a bond from the antibody-linker to the payload to form an antibody-linker-payload conjugate. In certain embodiments, a payload precursor is contacted with a linker precursor under conditions suitable for forming a bond from the payload to the linker. The resulting payload-linker is contacted with an antibody under conditions suitable for forming a bond from the payload-linker to the antibody to form an antibody-linker-payload conjugate. Suitable linkers for preparing the antibody conjugates are disclosed herein, and exemplary conditions for conjugation are described in the Examples below.

In some embodiments, an anti-ROR1 conjugate is prepared by contacting an anti-ROR1 antibody as disclosed herein with a linker precursor having a structure of any of LP1-LP39:

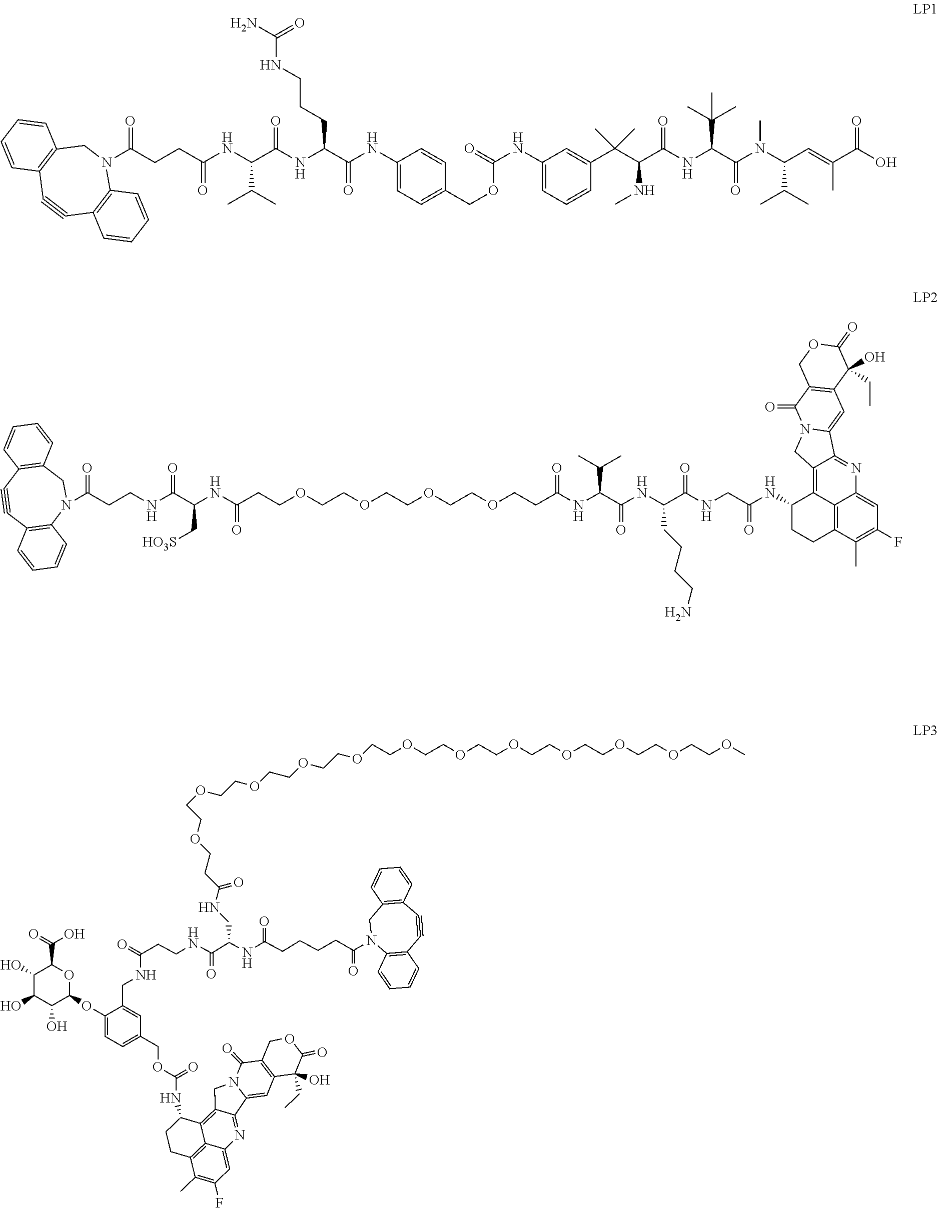

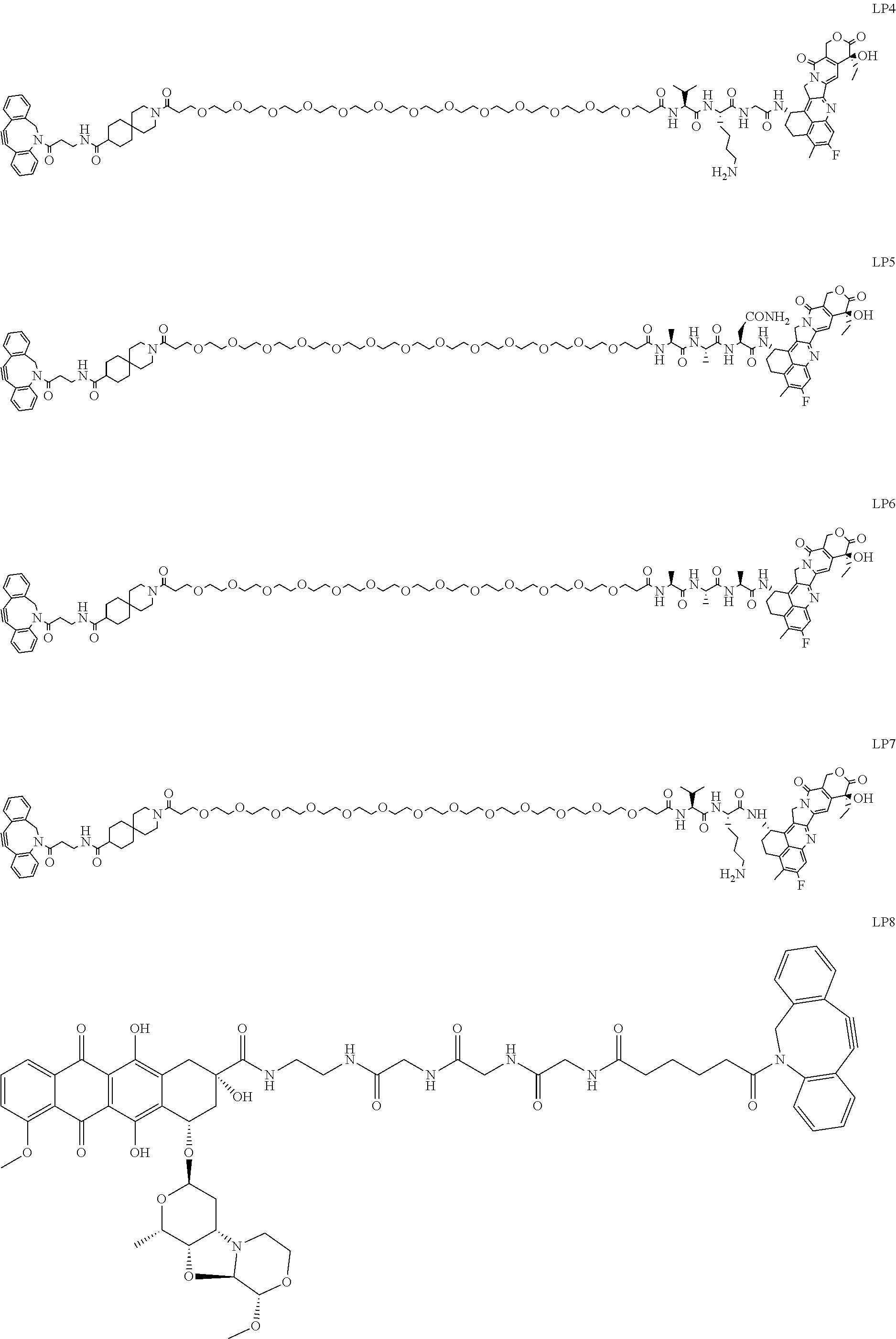

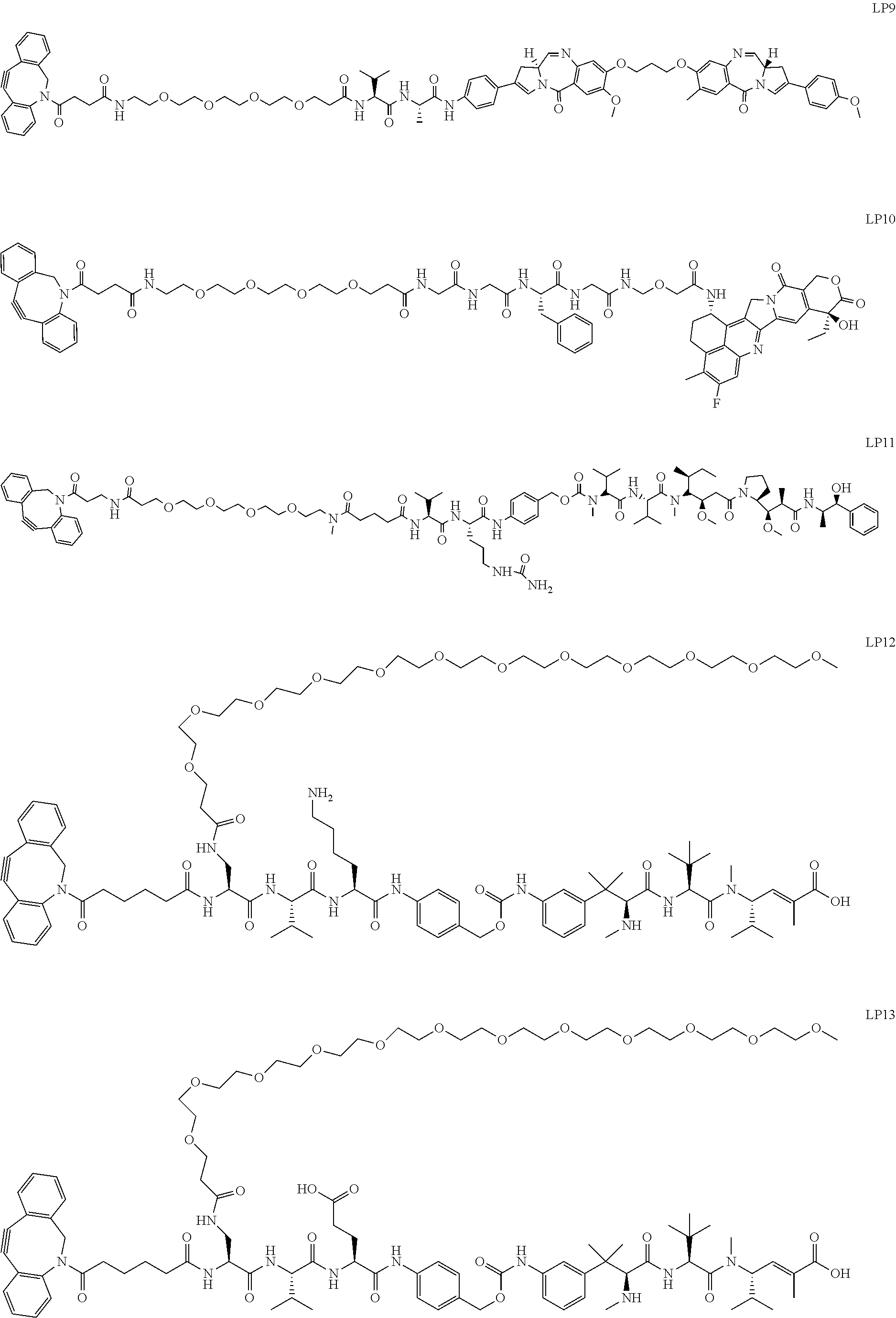

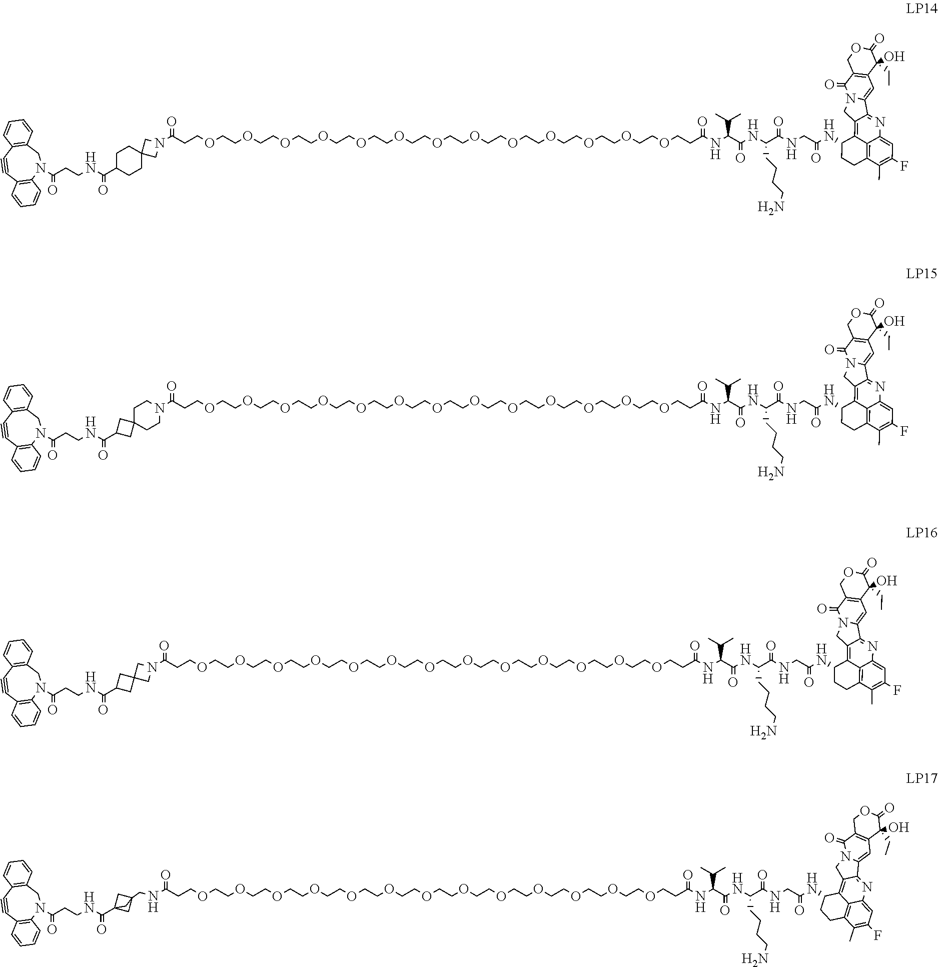

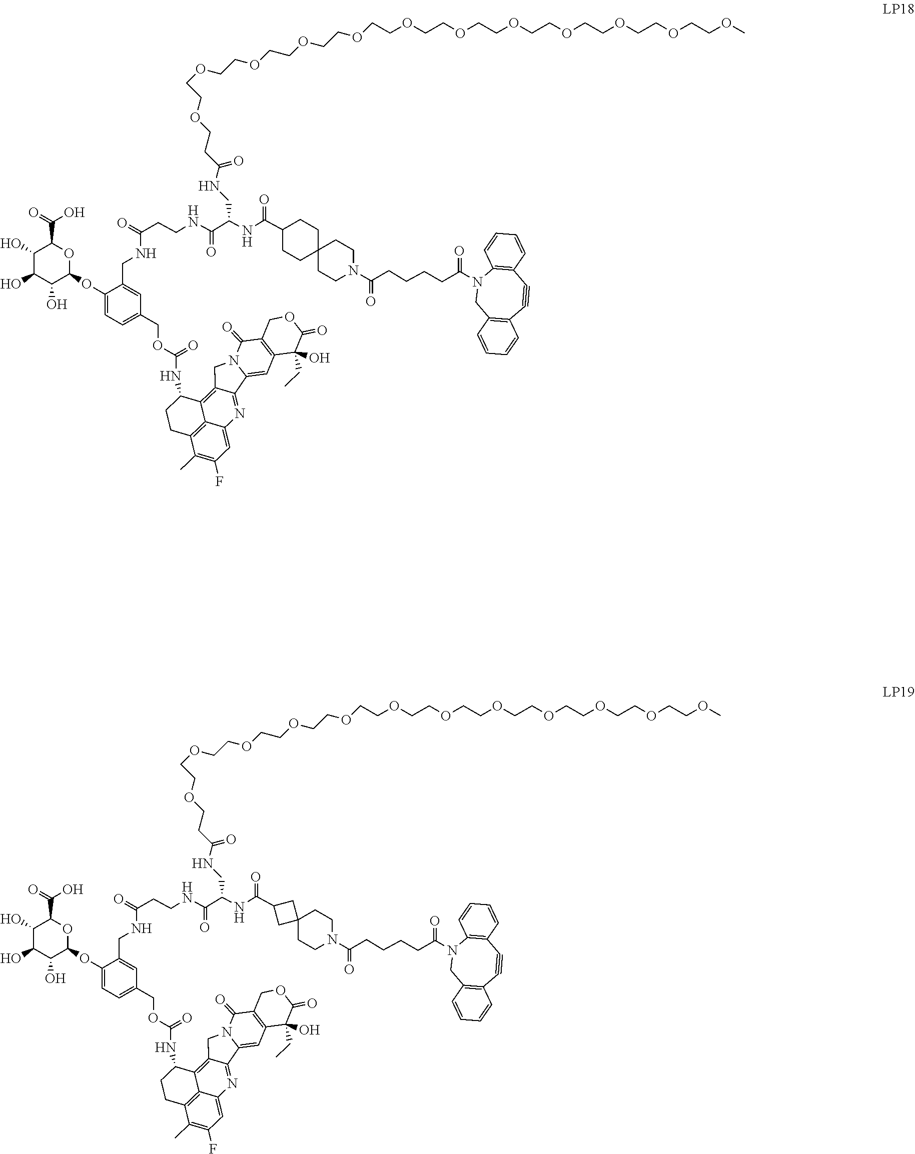

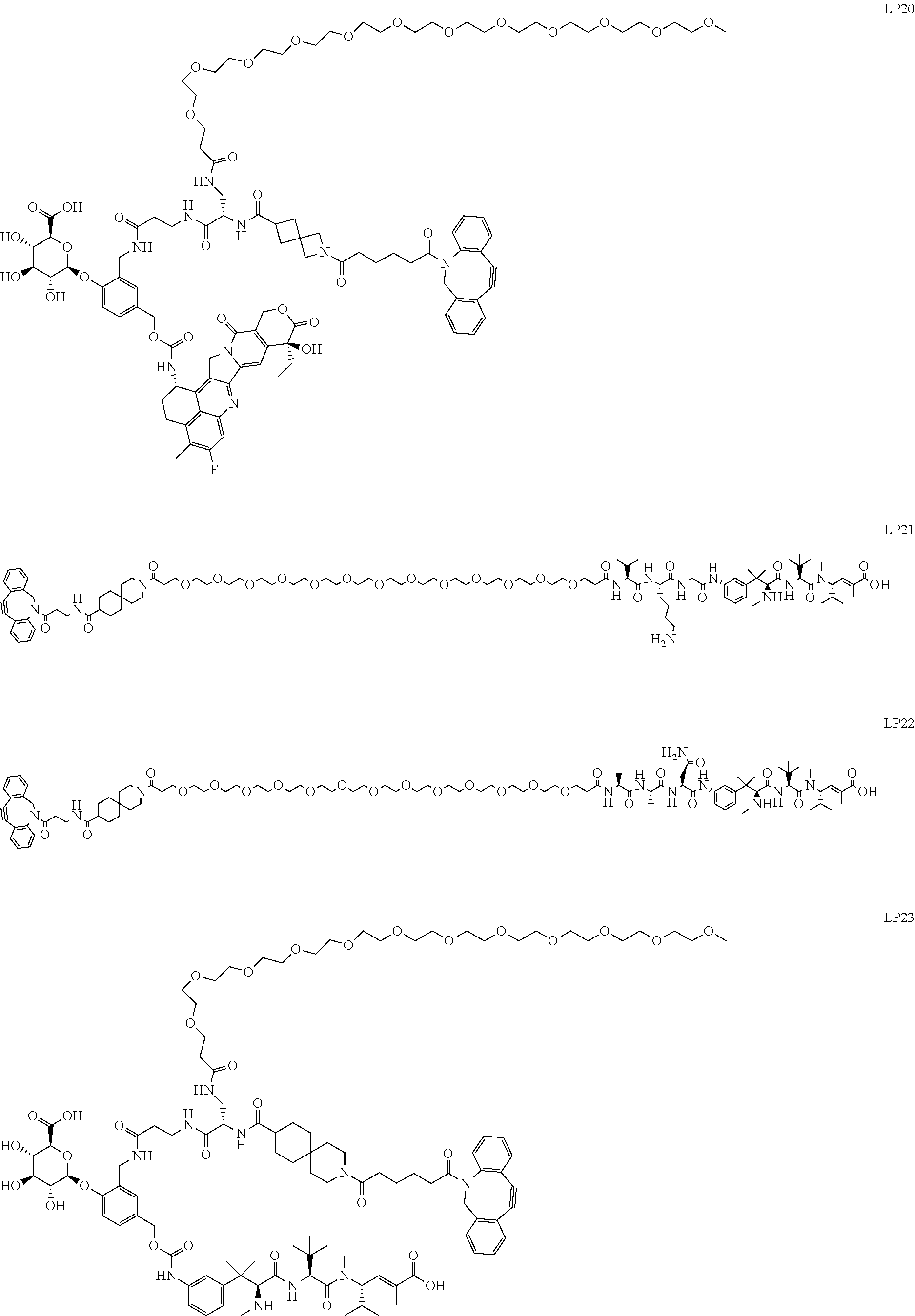

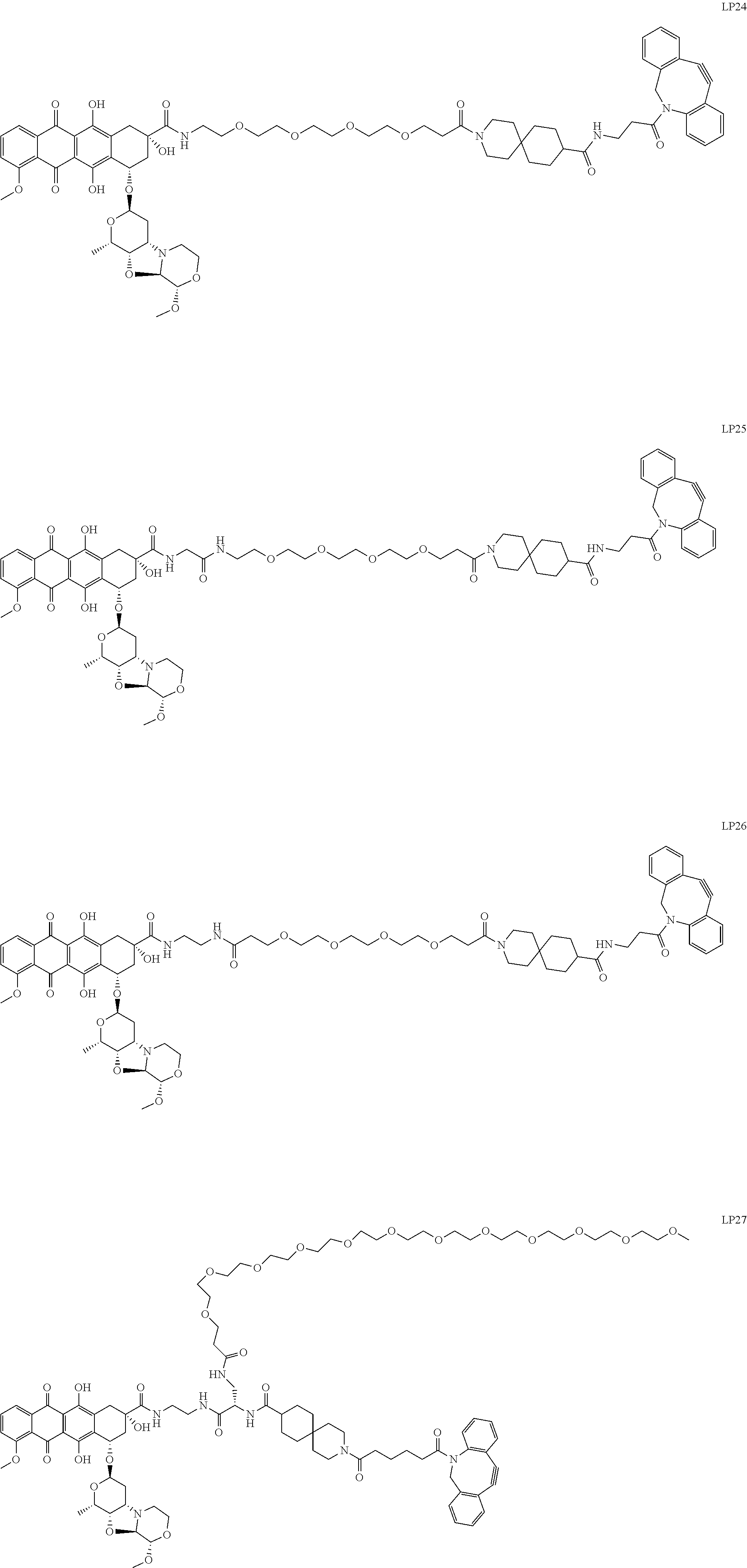

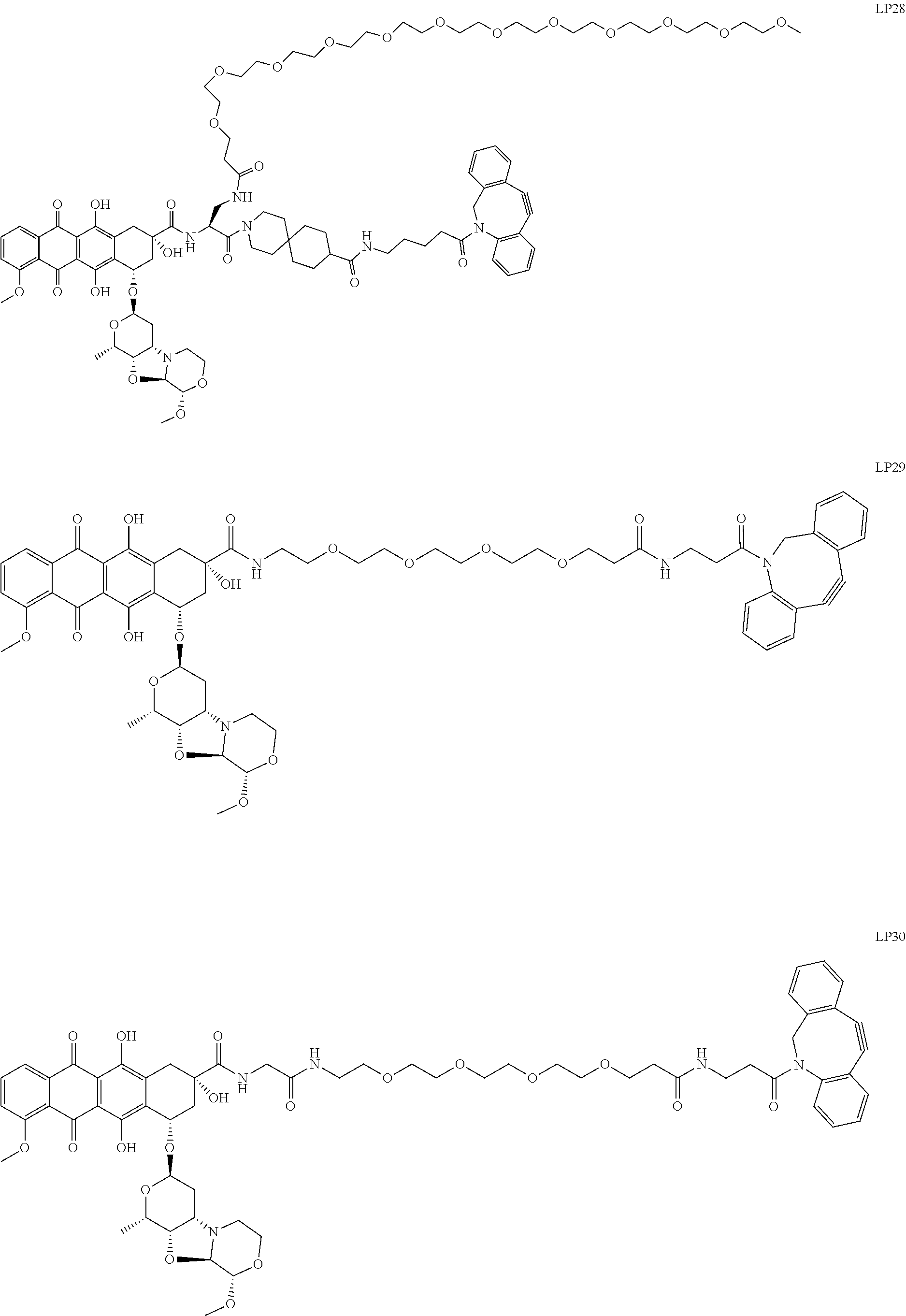

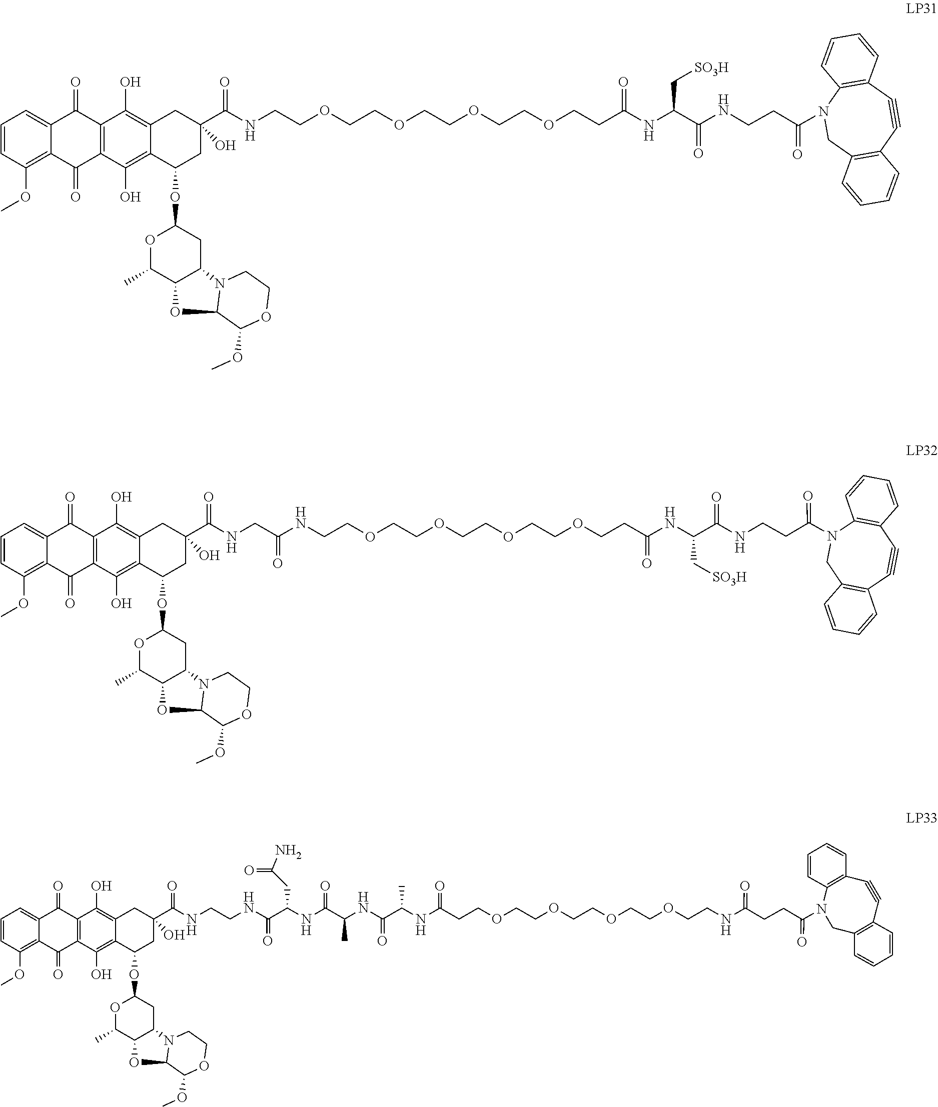

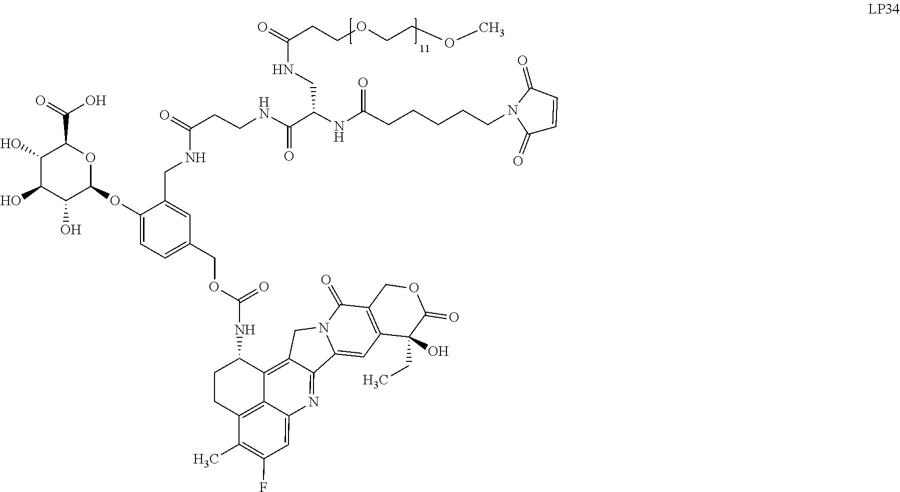

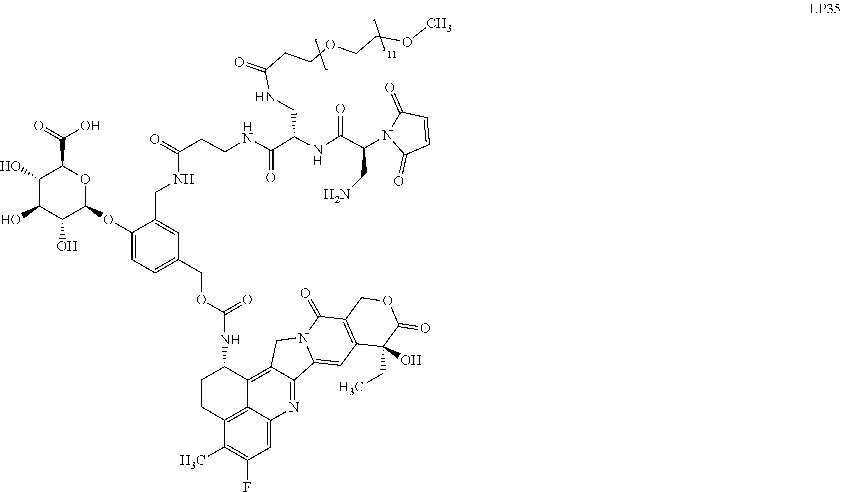

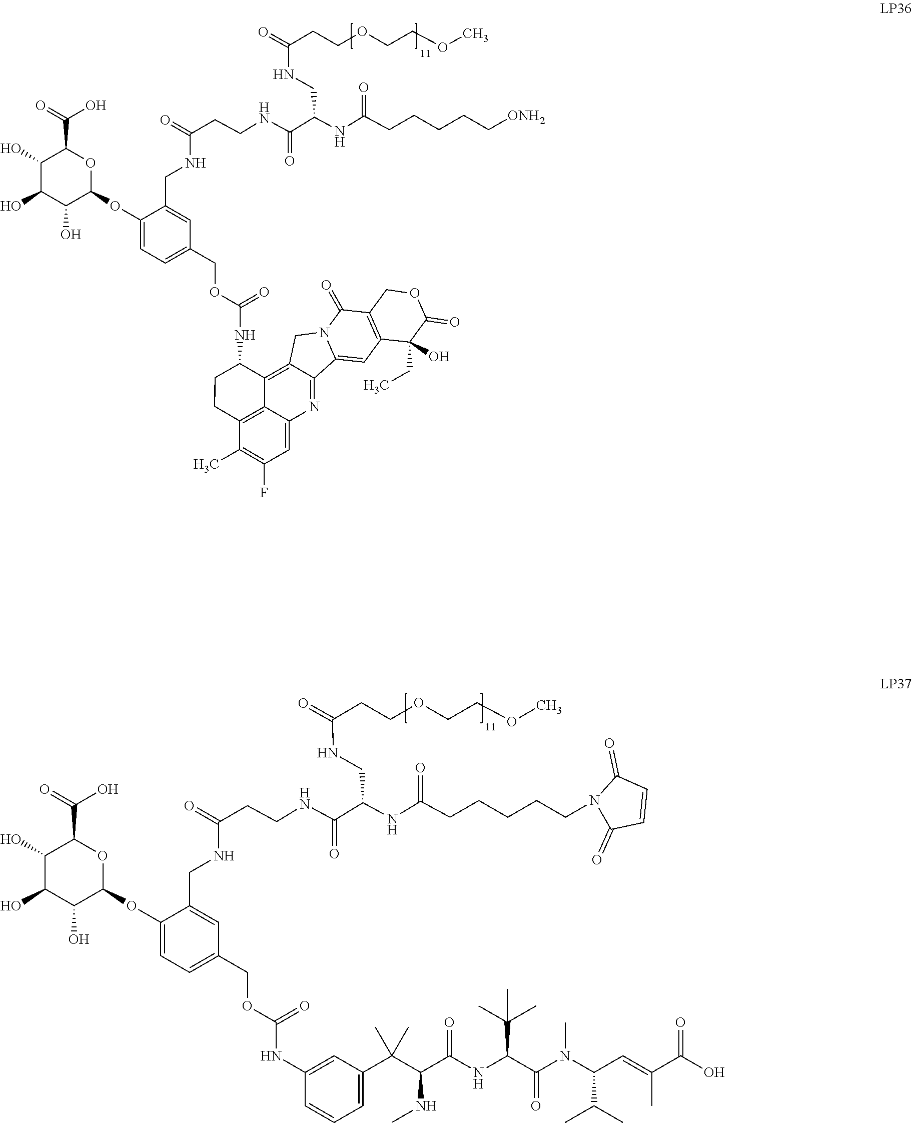

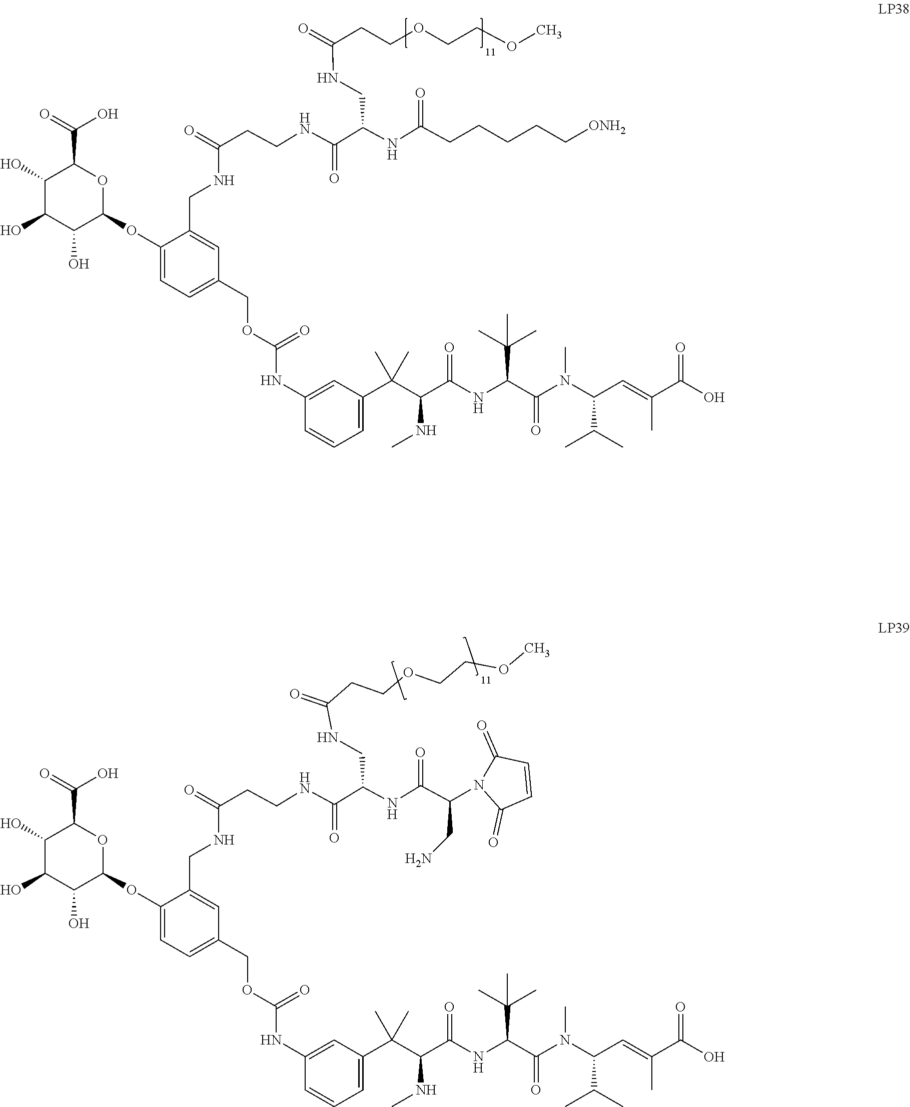

In some embodiments, an anti-ROR1 conjugate is prepared by contacting an anti-ROR1 antibody as disclosed herein with a linker precursor having a structure of any of LP1A-LP39A:

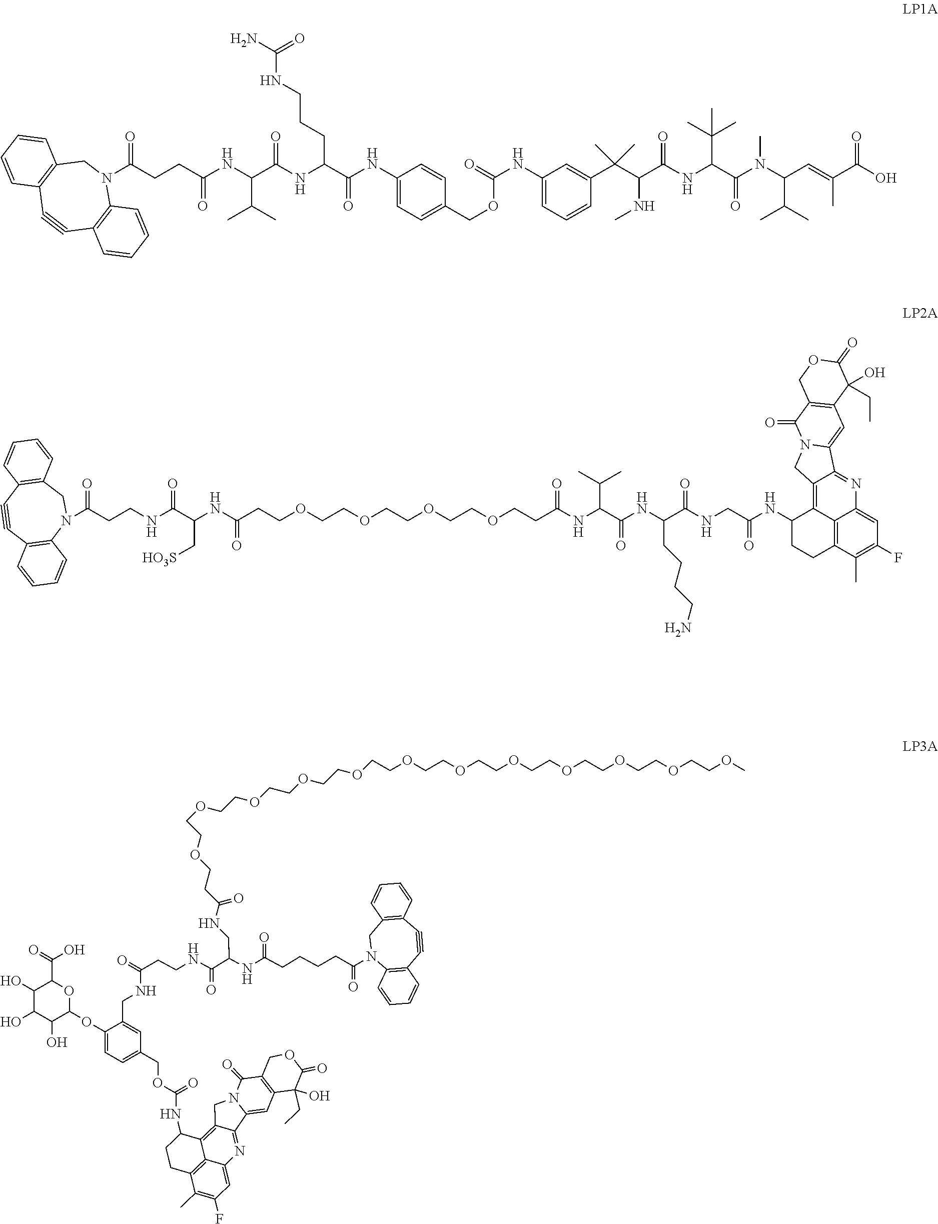

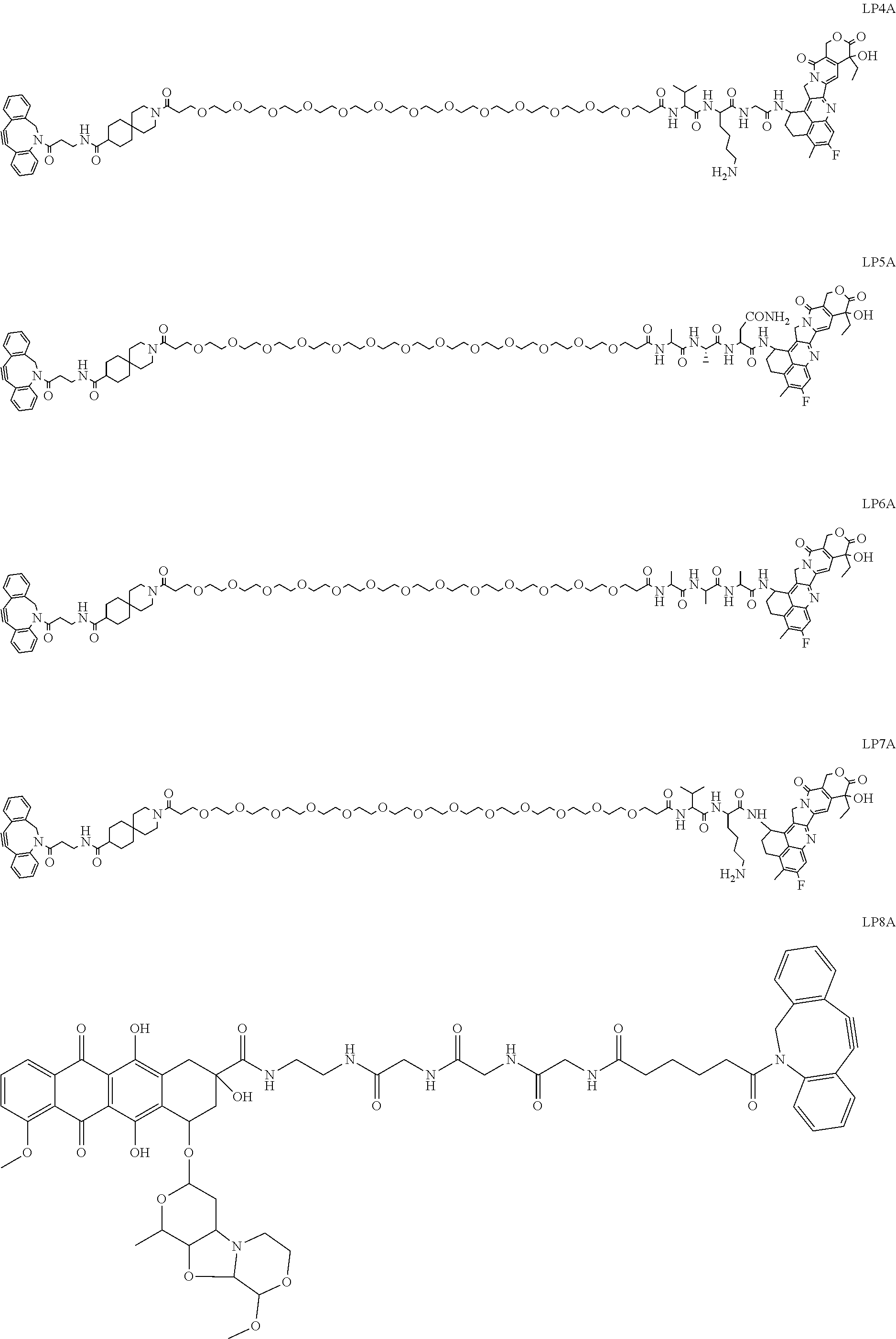

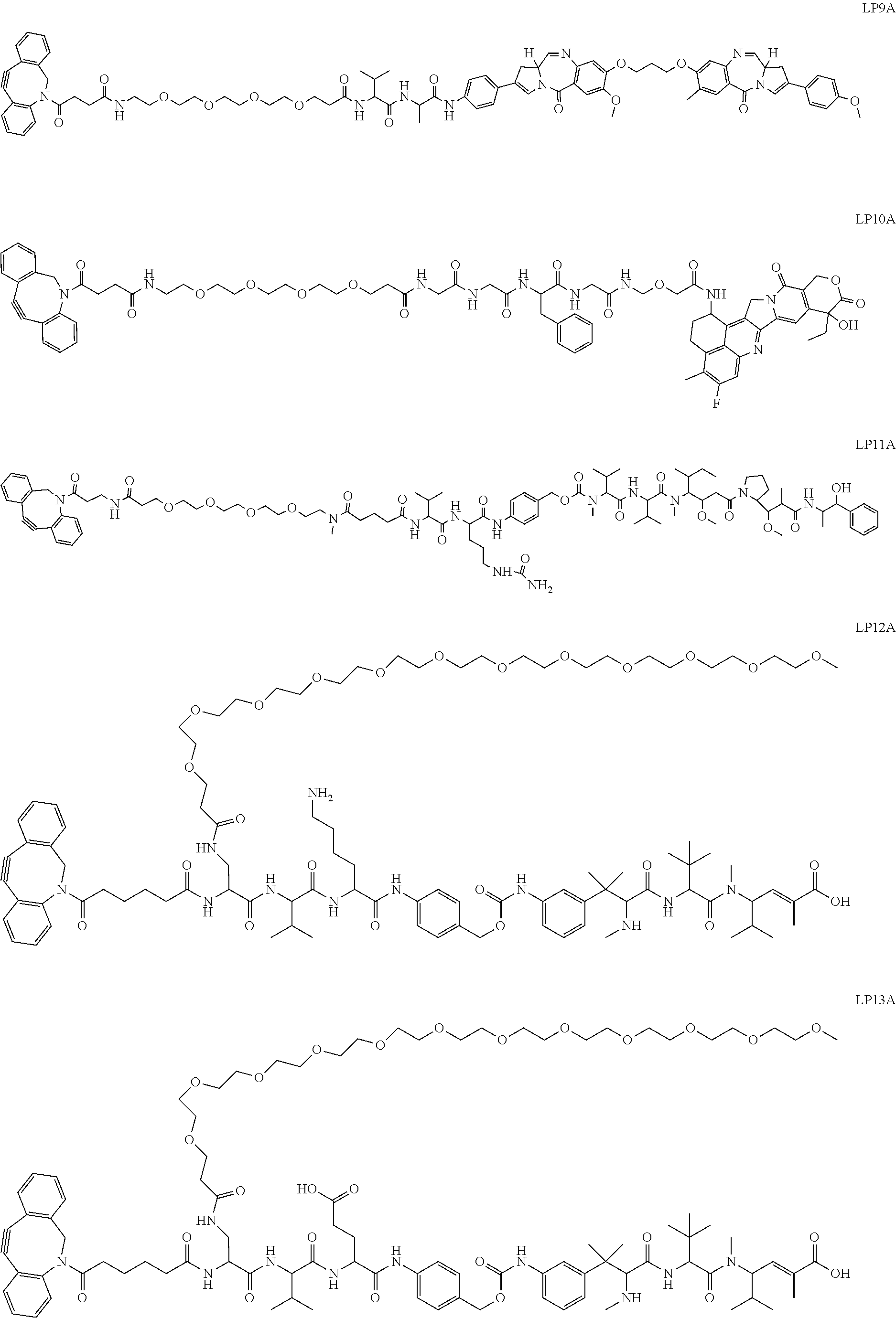

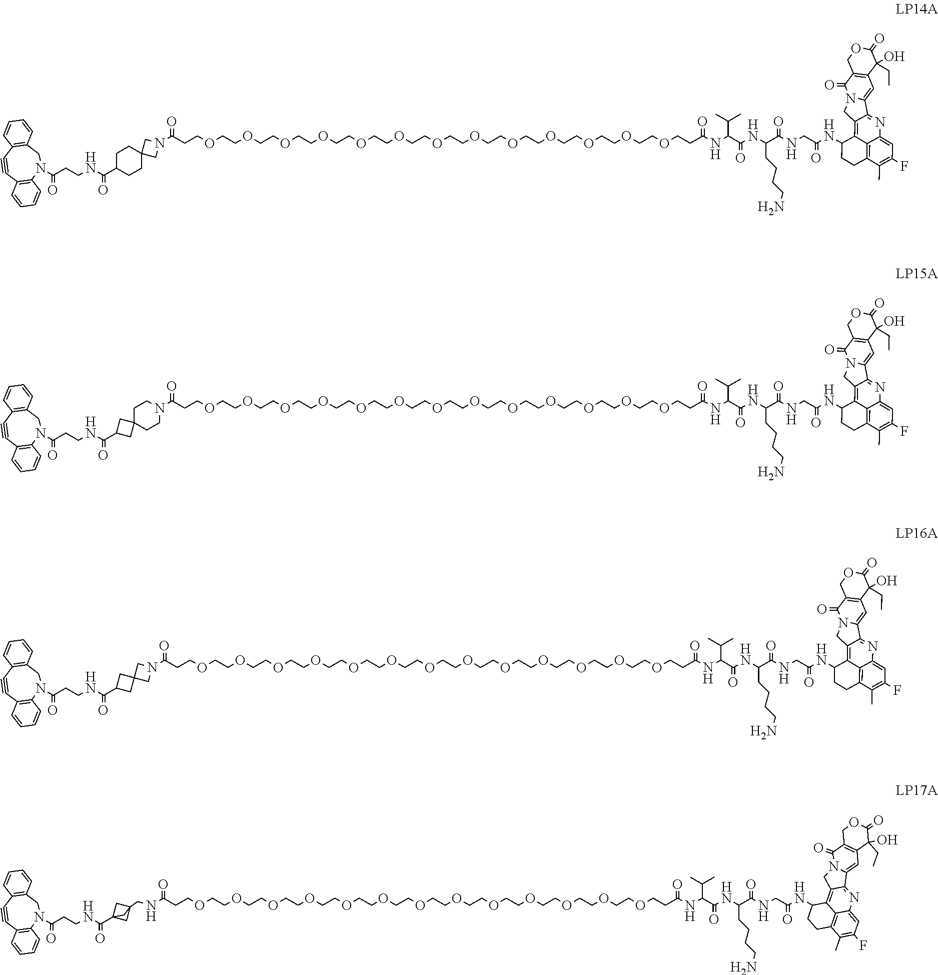

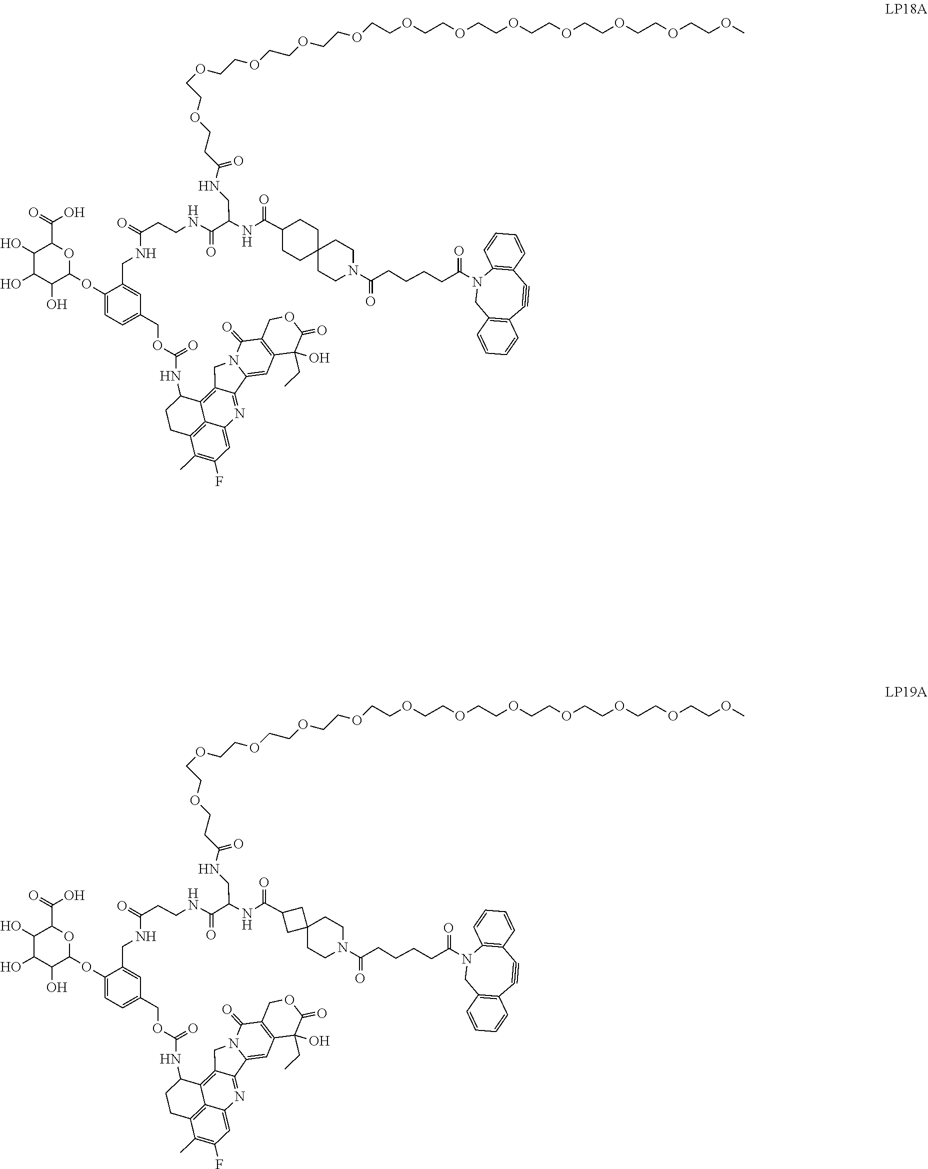 [SMILES: CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)C2CCC4(CC2)CCN(C(=O)CCCCC(=O)N2Cc5ccccc5C#Cc5ccccc52)CC4)c1)CC3 ; CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)C2CC4(CCN(C(=O)CCCCC(=O)N5Cc6ccccc6C#Cc6ccccc65)CC4)C2)c1)CC3]

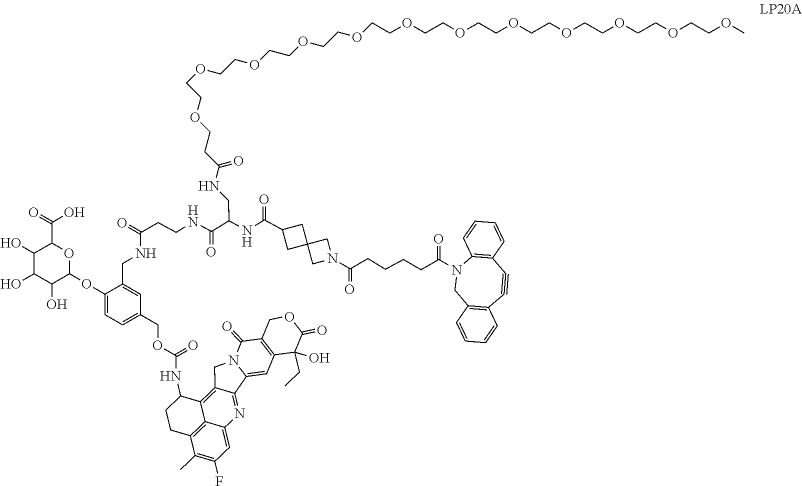 [SMILES: CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)C2CC4(C2)CN(C(=O)CCCCC(=O)N2Cc5ccccc5C#Cc5ccccc52)C4)c1)CC3]

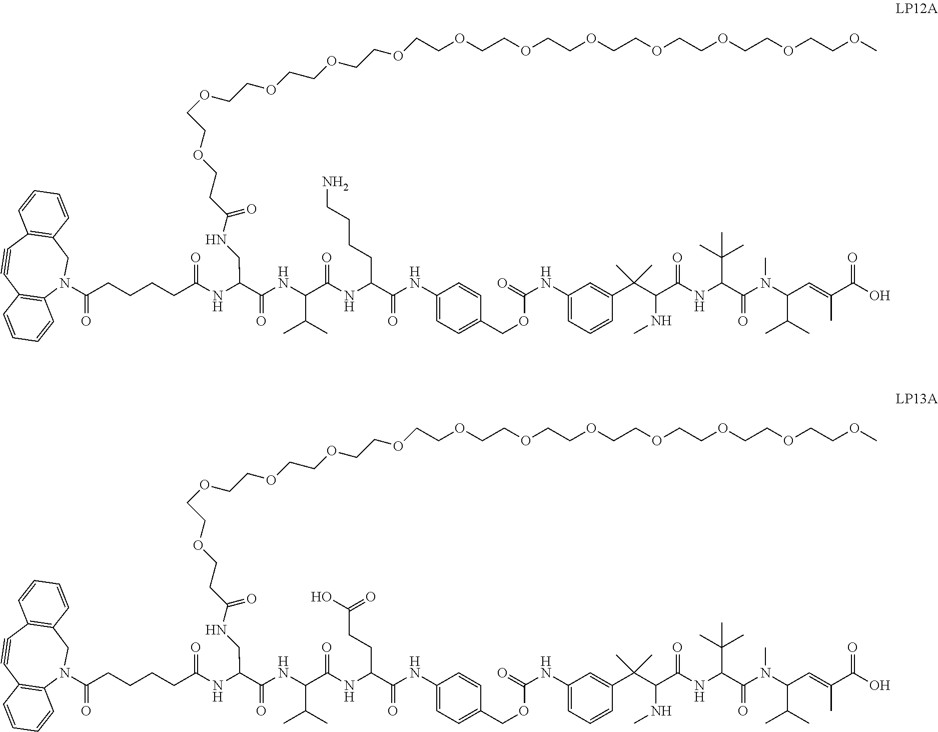 [SMILES: CNC(C(=O)NC(C(=O)N(C)C(/C=C(\C)C(=O)O)C(C)C)C(C)(C)C)C(C)(C)c1cccc(NC(=O)OCc2ccc(NC(=O)C(CCCCN)NC(=O)C(NC(=O)C(CNC(=O)CCOCCOCCOCCOCCOCCOCCOCCOCCOCCOCCOCCOC)NC(=O)CCCCC(=O)N3Cc4ccccc4C#Cc4ccccc43)C(C)C)cc2)c1 ; CNC(C(=O)NC(C(=O)N(C)C(/C=C(\C)C(=O)O)C(C)C)C(C)(C)C)C(C)(C)c1cccc(NC(=O)OCc2ccc(NC(=O)C(CCC(=O)O)NC(=O)C(NC(=O)C(CNC(=O)CCOCCOCCOCCOCCOCCOCCOCCOCCOCCOCCOCCOC)NC(=O)CCCCC(=O)N3Cc4ccccc4C#Cc4ccccc43)C(C)C)cc2)c1]

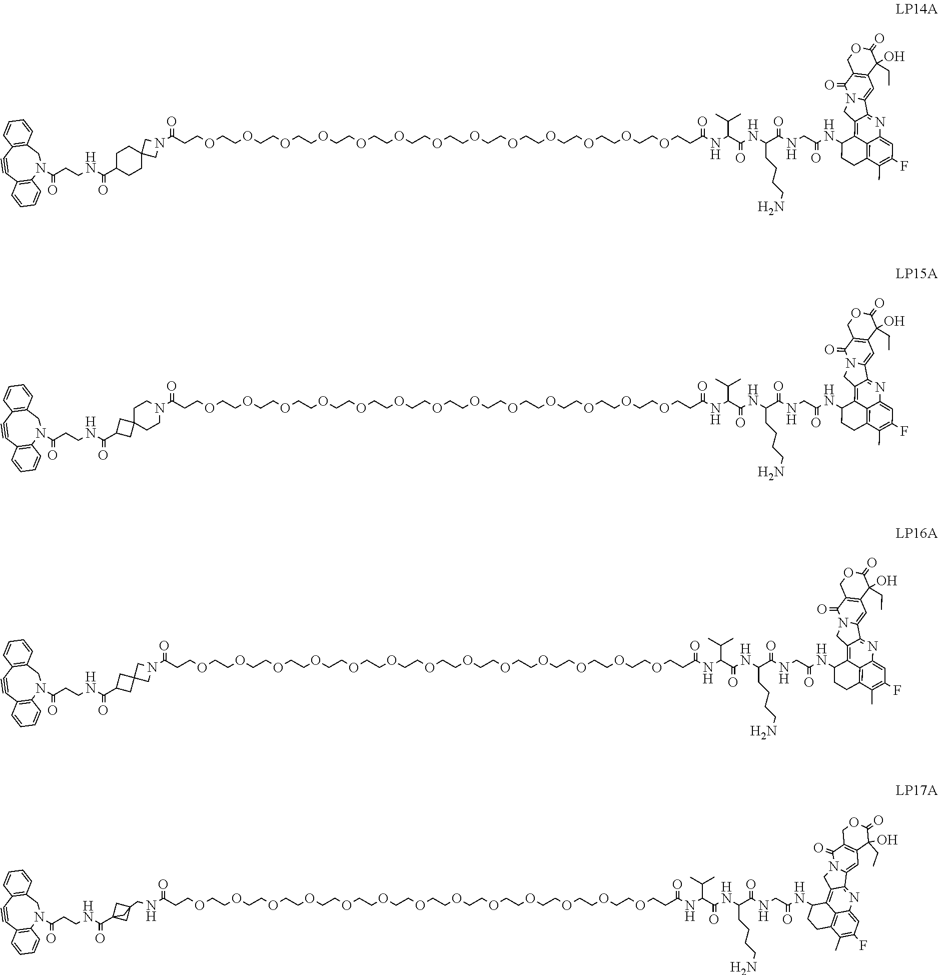

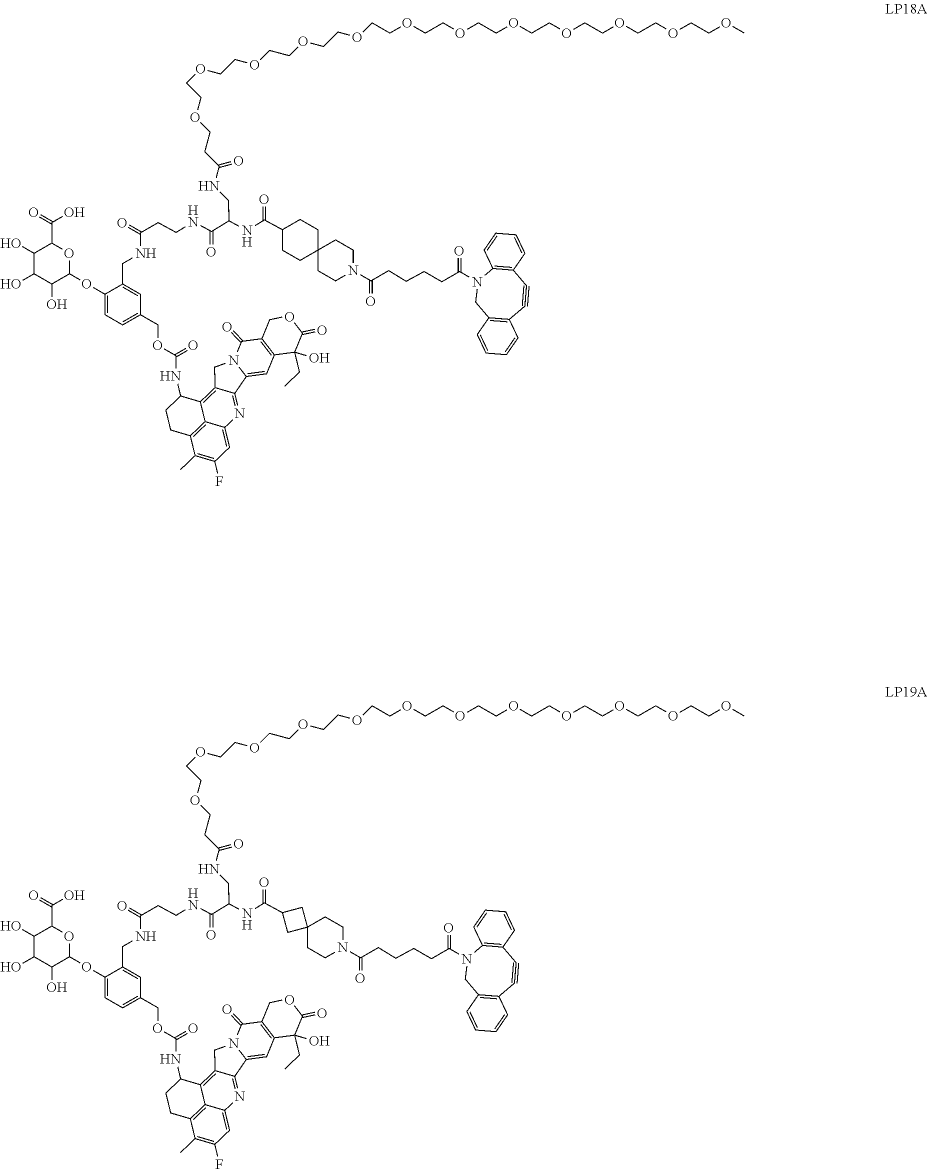 [SMILES: CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)C2CCC4(CC2)CCN(C(=O)CCCCC(=O)N2Cc5ccccc5C#Cc5ccccc52)CC4)c1)CC3 ; CCC1(O)C(=O)OCc2c1cc1n(c2=O)Cc2c-1nc1cc(F)c(C)c3c1c2C(NC(=O)OCc1ccc(OC2OC(C(=O)O)C(O)C(O)C2O)c(CNC(=O)CCNC(=O)C(CNC(=O)CCOCCOCCOCCOCCOCCOCCOCCOCCOCCOCCOCCOC)NC(=O)C2CC4(CCN(C(=O)CCCCC(=O)N5Cc6ccccc6C#Cc6ccccc65)CC4)C2)c1)CC3]

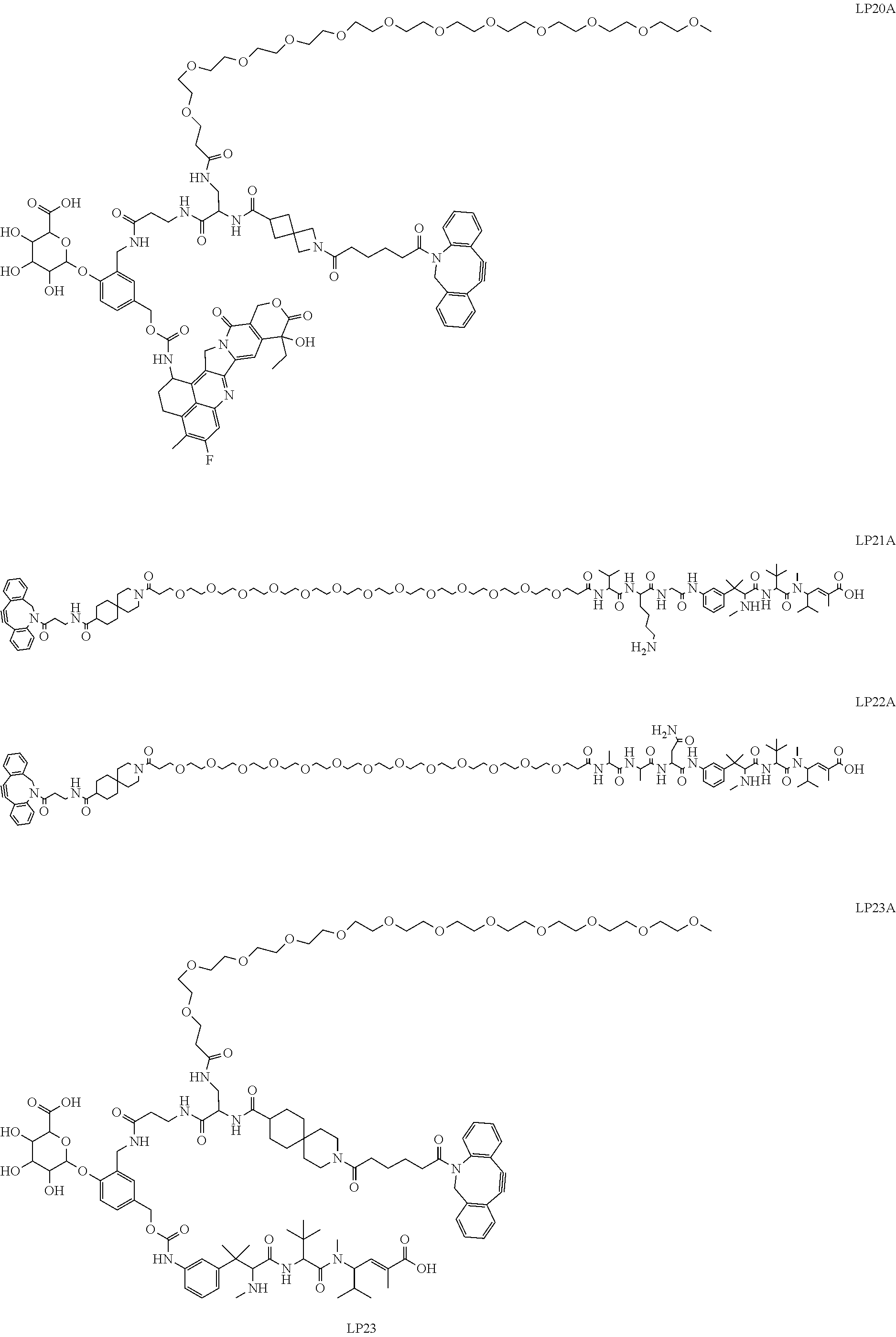

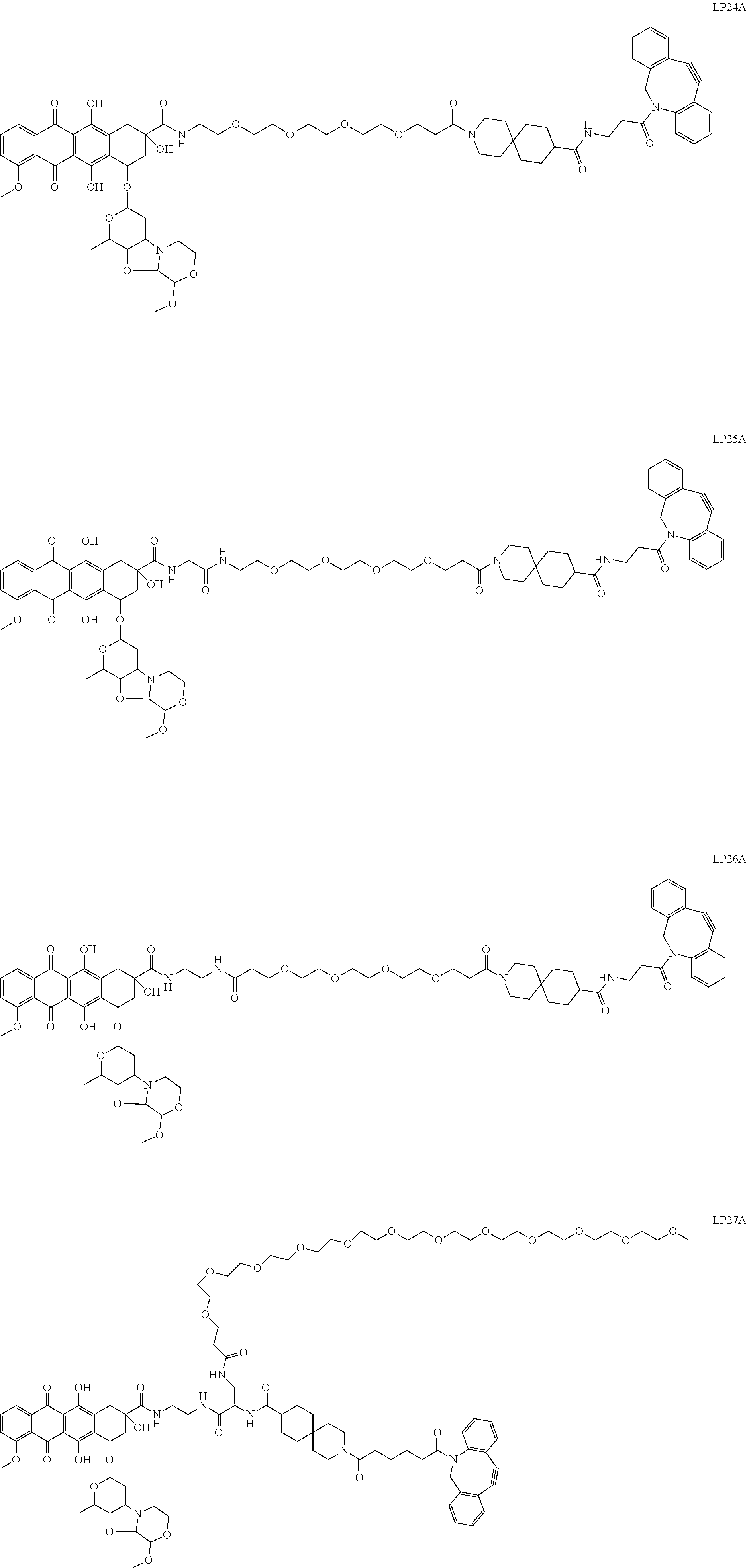

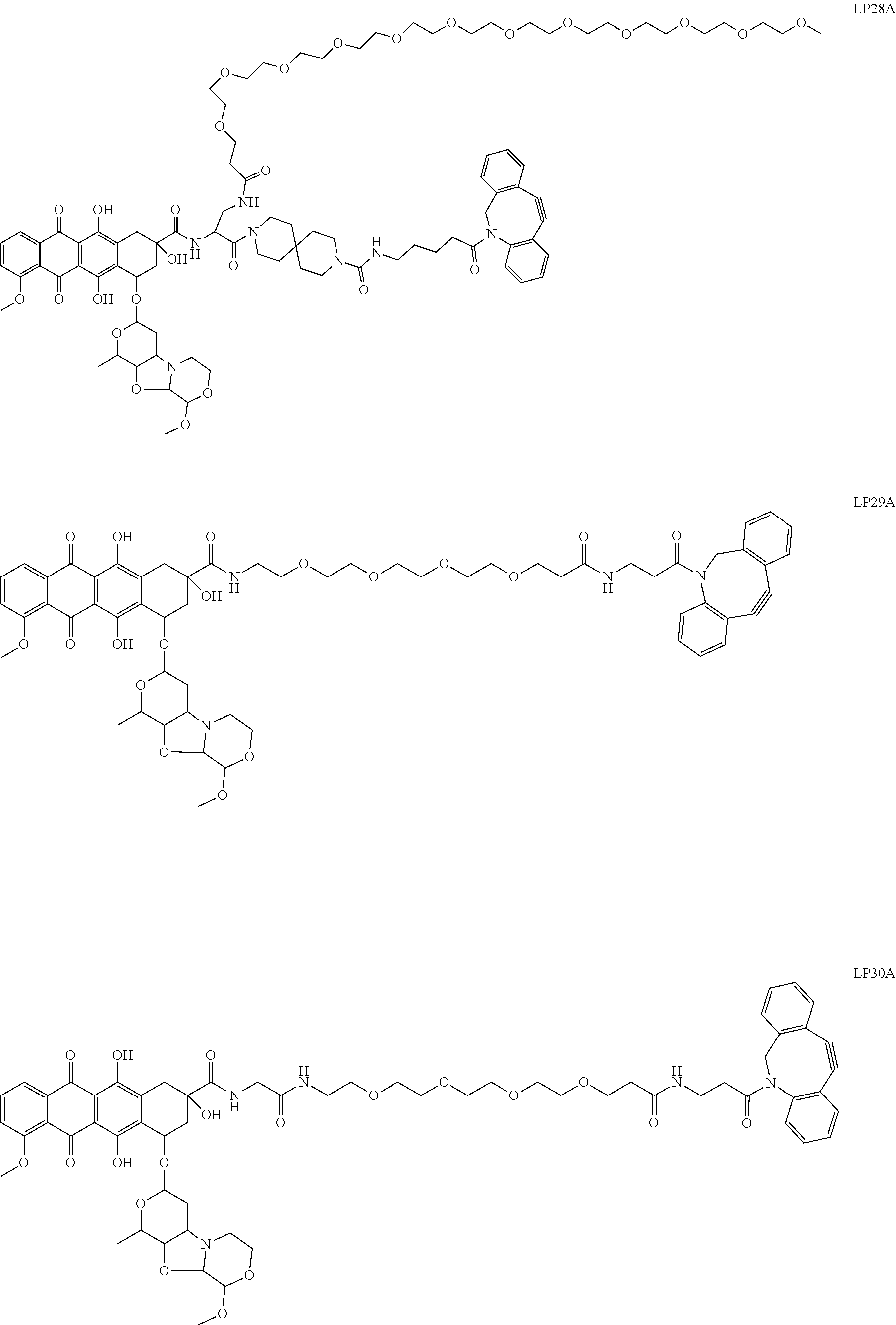 [SMILES: COCCOCCOCCOCCOCCOCCOCCOCCOCCOCCOCCOCCC(=O)NCC(NC(=O)C1(O)Cc2c(O)c3c(c(O)c2C(OC2CC4C(OC5C(OC)OCCN45)C(C)O2)C1)C(=O)c1c(OC)cccc1C3=O)C(=O)N1CCC2(CCN(C(=O)NCCCCC(=O)N3Cc4ccccc4C#Cc4ccccc43)CC2)CC1 ; COc1cccc2c1C(=O)c1c(O)c3c(c(O)c1C2=O)CC(O)(C(=O)NCCOCCOCCOCCOCCC(=O)NCCC(=O)N1Cc2ccccc2C#Cc2ccccc21)CC3OC1CC2C(OC3C(OC)OCCN23)C(C)O1 ; COc1cccc2c1C(=O)c1c(O)c3c(c(O)c1C2=O)CC(O)(C(=O)NCC(=O)NCCOCCOCCOCCOCCC(=O)NCCC(=O)N1Cc2ccccc2C#Cc2ccccc21)CC3OC1CC2C(OC3C(OC)OCCN23)C(C)O1]

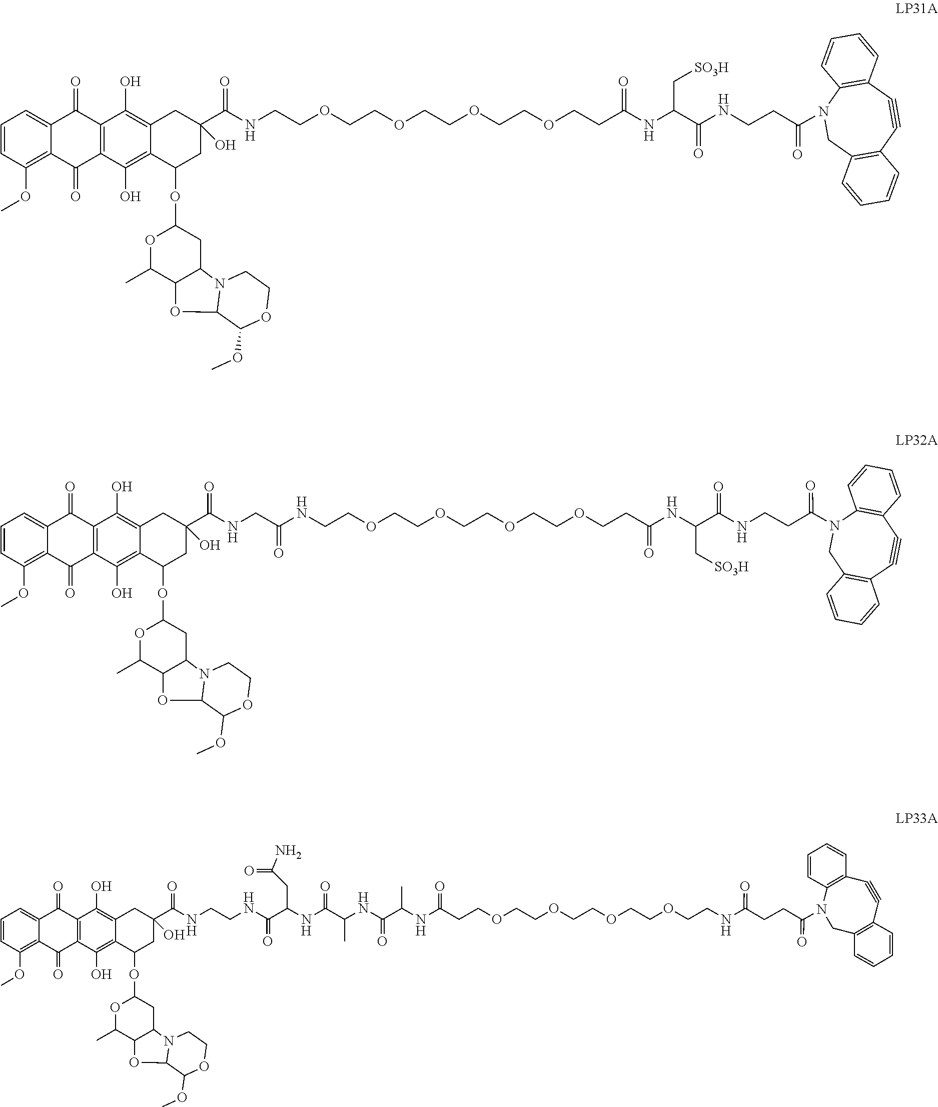 [SMILES: COc1cccc2c1C(=O)c1c(O)c3c(c(O)c1C2=O)CC(O)(C(=O)NCCOCCOCCOCCOCCC(=O)NC(CS(=O)(=O)O)C(=O)NCCC(=O)N1Cc2ccccc2C#Cc2ccccc21)CC3OC1CC2C(OC3[C@@H](OC)OCCN23)C(C)O1 ; COc1cccc2c1C(=O)c1c(O)c3c(c(O)c1C2=O)CC(O)(C(=O)NCC(=O)NCCOCCOCCOCCOCCC(=O)NC(CS(=O)(=O)O)C(=O)NCCC(=O)N1Cc2ccccc2C#Cc2ccccc21)CC3OC1CC2C(OC3C(OC)OCCN23)C(C)O1 ; COc1cccc2c1C(=O)c1c(O)c3c(c(O)c1C2=O)CC(O)(C(=O)NCCNC(=O)C(CC(N)=O)NC(=O)C(C)NC(=O)C(C)NC(=O)CCOCCOCCOCCOCCNC(=O)CCC(=O)N1Cc2ccccc2C#Cc2ccccc21)CC3OC1CC2C(OC3C(OC)OCCN23)C(C)O1]

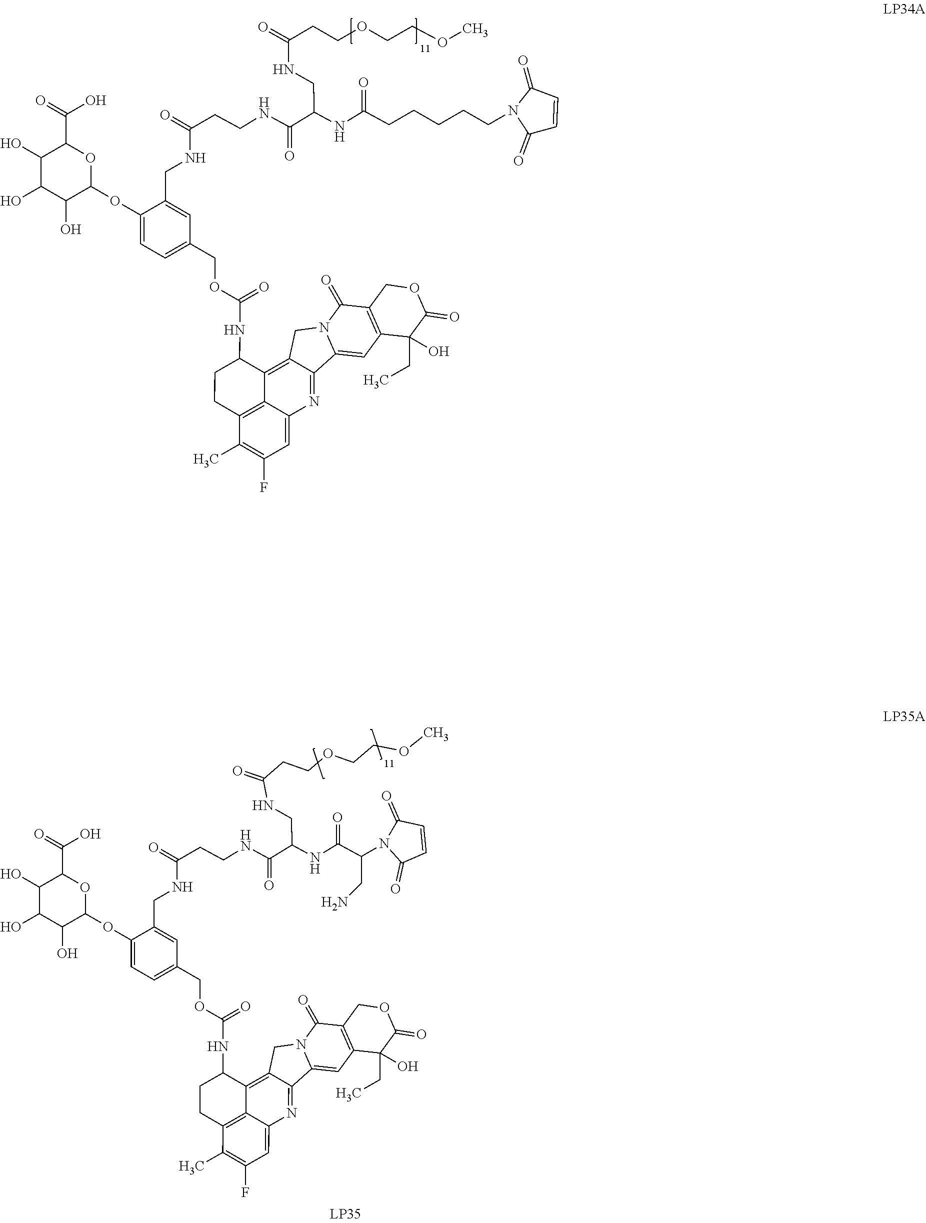

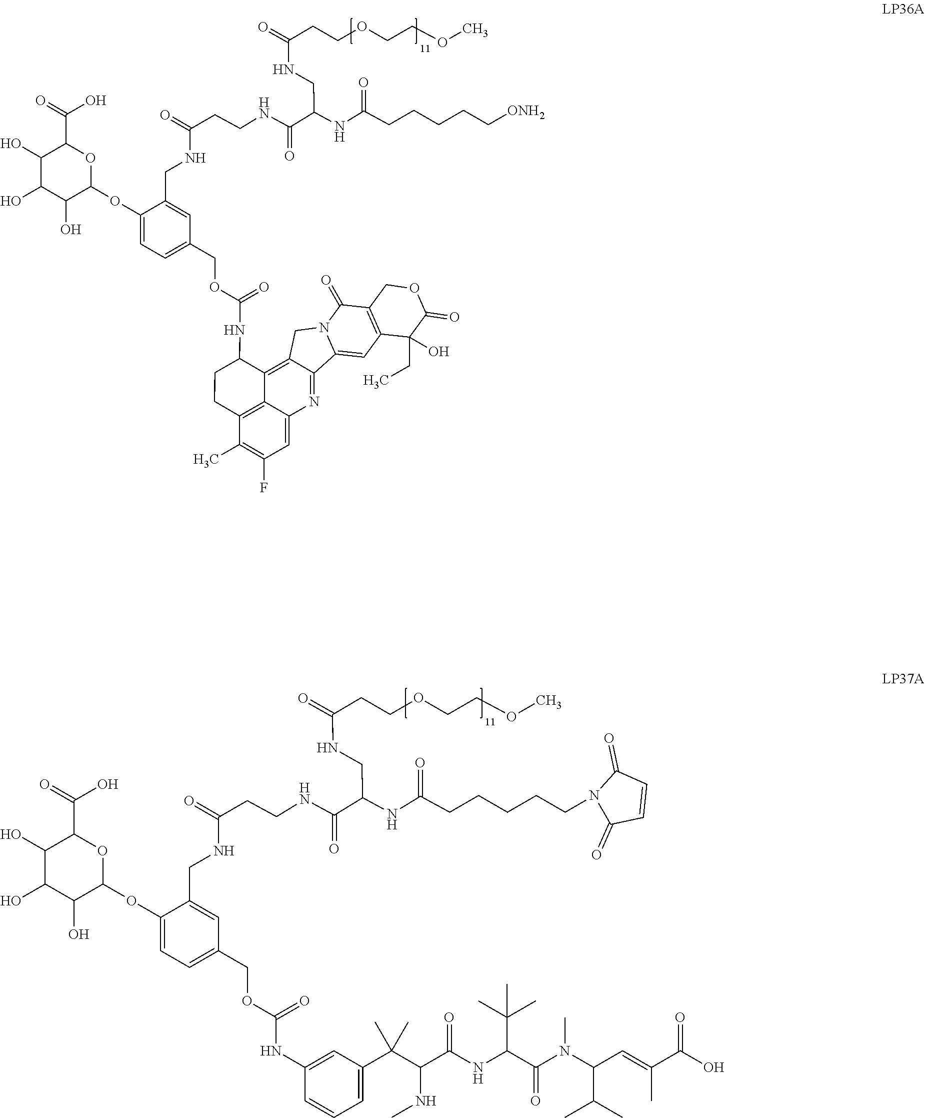

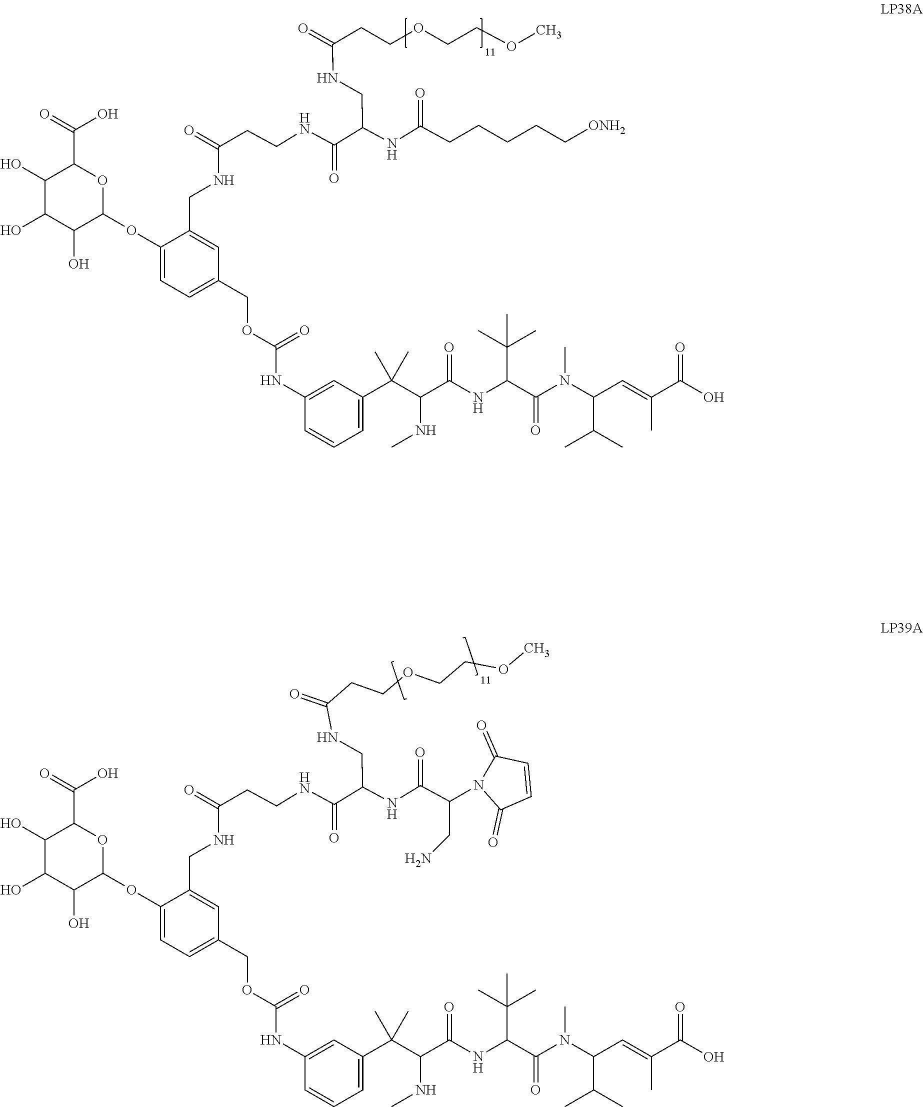

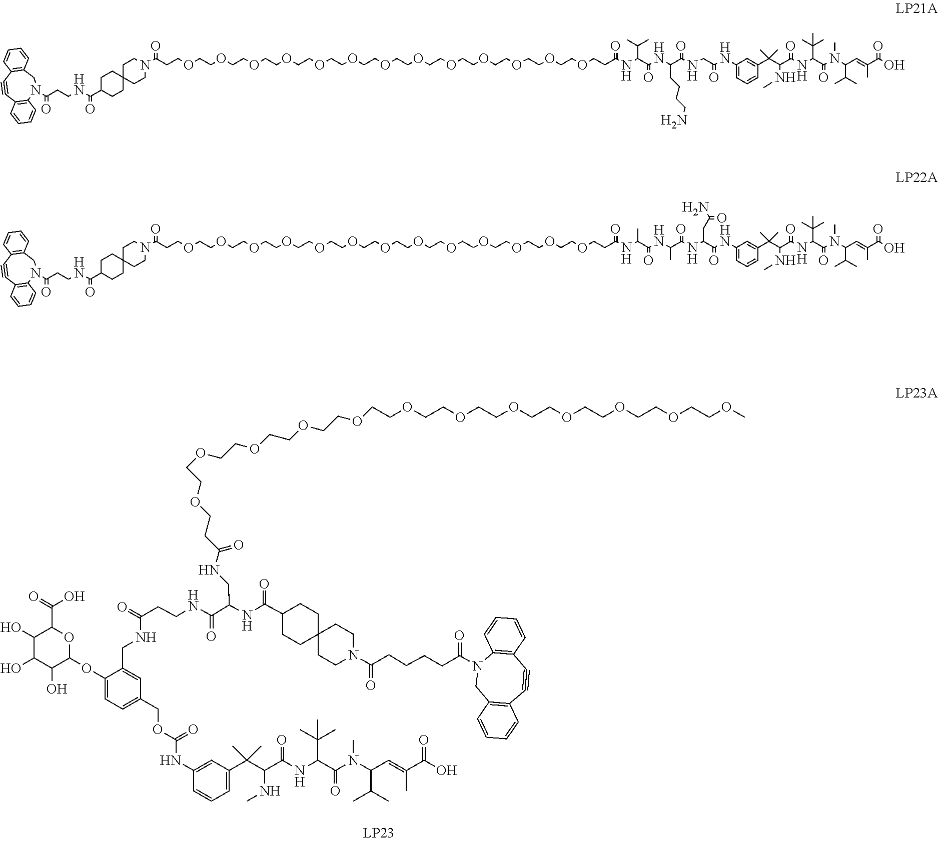

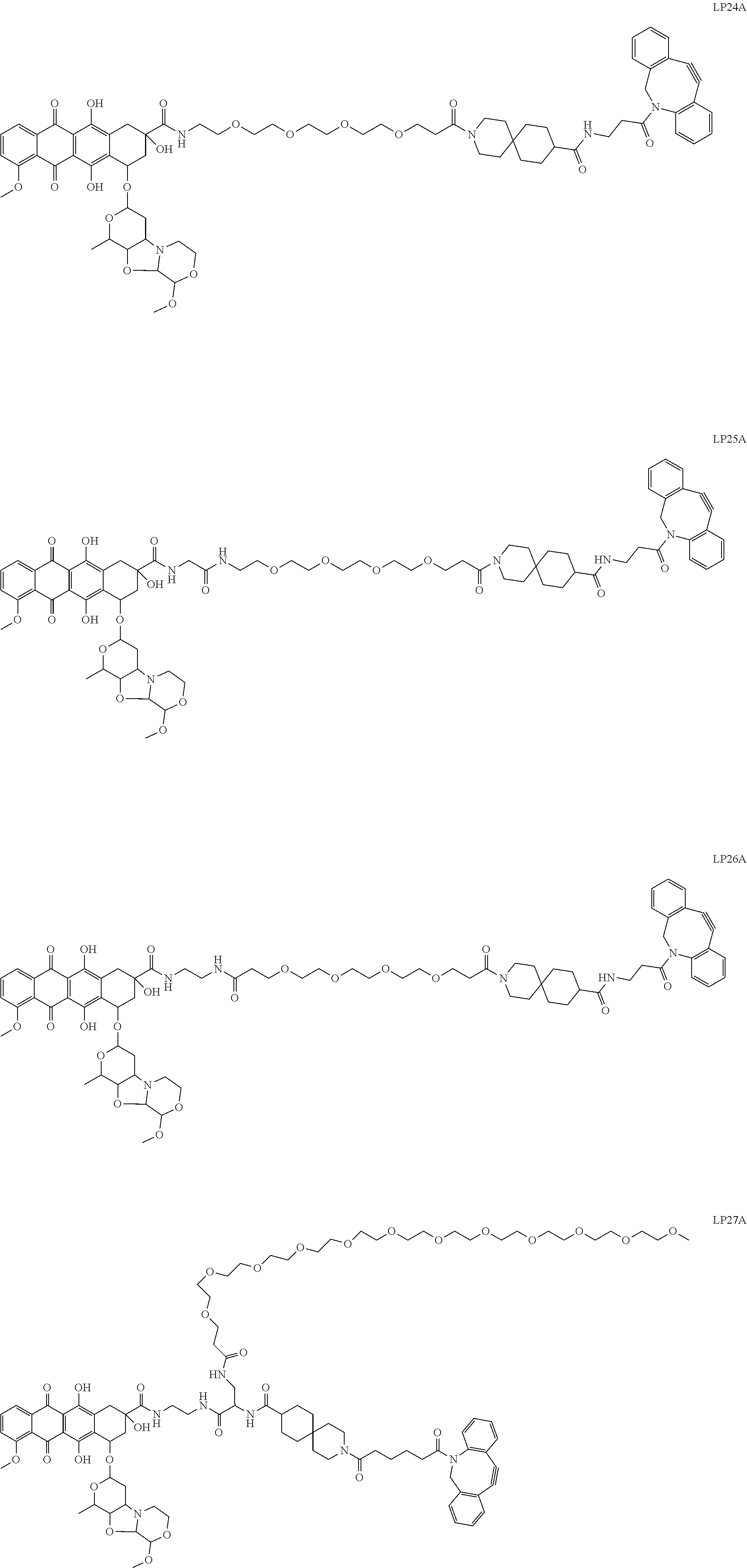

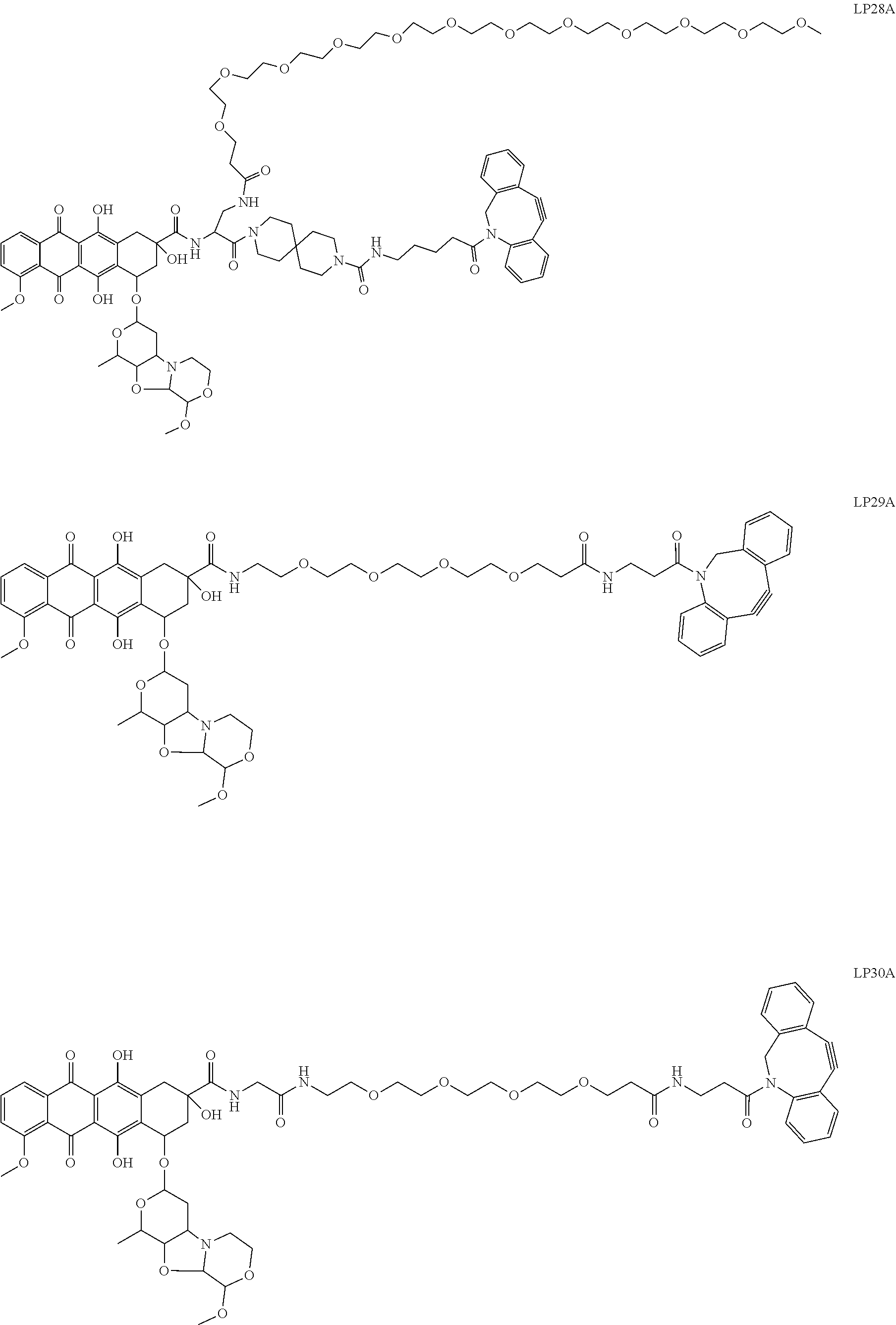 [SMILES: COCCOCCOCCOCCOCCOCCOCCOCCOCCOCCOCCOCCC(=O)NCC(NC(=O)C1(O)Cc2c(O)c3c(c(O)c2C(OC2CC4C(OC5C(OC)OCCN45)C(C)O2)C1)C(=O)c1c(OC)cccc1C3=O)C(=O)N1CCC2(CCN(C(=O)NCCCCC(=O)N3Cc4ccccc4C#Cc4ccccc43)CC2)CC1 ; COc1cccc2c1C(=O)c1c(O)c3c(c(O)c1C2=O)CC(O)(C(=O)NCCOCCOCCOCCOCCC(=O)NCCC(=O)N1Cc2ccccc2C#Cc2ccccc21)CC3OC1CC2C(OC3C(OC)OCCN23)C(C)O1 ; COc1cccc2c1C(=O)c1c(O)c3c(c(O)c1C2=O)CC(O)(C(=O)NCC(=O)NCCOCCOCCOCCOCCC(=O)NCCC(=O)N1Cc2ccccc2C#Cc2ccccc21)CC3OC1CC2C(OC3C(OC)OCCN23)C(C)O1]

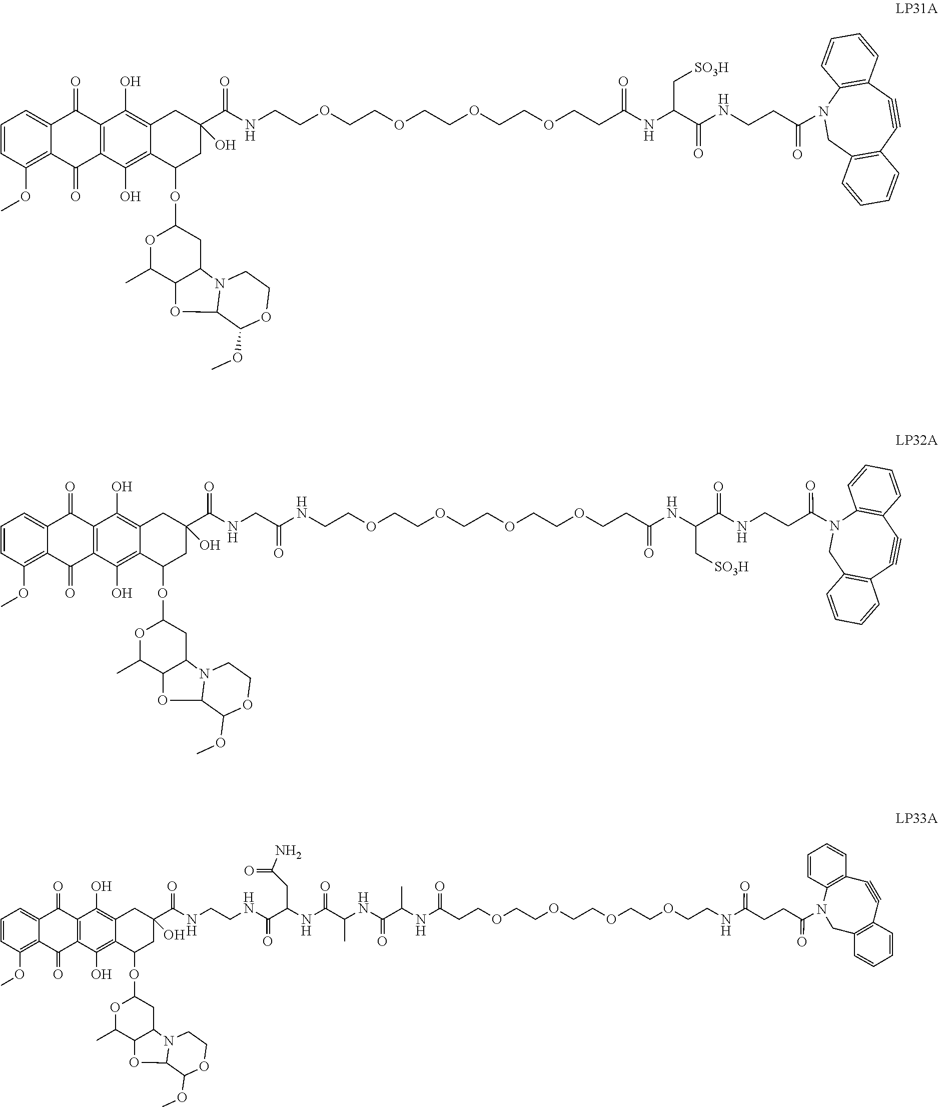 [SMILES: COc1cccc2c1C(=O)c1c(O)c3c(c(O)c1C2=O)CC(O)(C(=O)NCCOCCOCCOCCOCCC(=O)NC(CS(=O)(=O)O)C(=O)NCCC(=O)N1Cc2ccccc2C#Cc2ccccc21)CC3OC1CC2C(OC3[C@@H](OC)OCCN23)C(C)O1 ; COc1cccc2c1C(=O)c1c(O)c3c(c(O)c1C2=O)CC(O)(C(=O)NCC(=O)NCCOCCOCCOCCOCCC(=O)NC(CS(=O)(=O)O)C(=O)NCCC(=O)N1Cc2ccccc2C#Cc2ccccc21)CC3OC1CC2C(OC3C(OC)OCCN23)C(C)O1 ; COc1cccc2c1C(=O)c1c(O)c3c(c(O)c1C2=O)CC(O)(C(=O)NCCNC(=O)C(CC(N)=O)NC(=O)C(C)NC(=O)C(C)NC(=O)CCOCCOCCOCCOCCNC(=O)CCC(=O)N1Cc2ccccc2C#Cc2ccccc21)CC3OC1CC2C(OC3C(OC)OCCN23)C(C)O1]

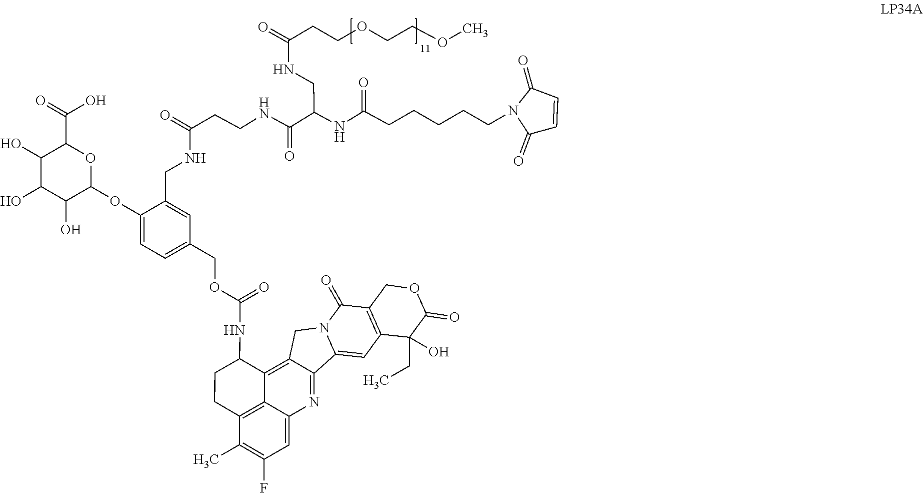

### 13. Vectors, Host Cells, and Recombinant Methods

The invention also provides isolated nucleic acids encoding anti-ROR1 antibodies, vectors and host cells comprising the nucleic acids, and recombinant techniques for the production of the antibodies.

The ROR1 antibodies can be prepared according to techniques apparent to the person of skill. In certain embodiments, the ROR1 antibodies are prepared from isolated nucleic acids encoding anti-ROR1 antibodies, vectors, and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a 397 transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-ROR1 antibody-encoding vectors. Saccharomyces cerevisiae, or common baker's yeast, is a commonly used lower eukaryotic host microorganism. However, a number of other genera, species, and strains are available and useful, such as Spodoptera frugiperda (e.g., SF9), Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-ROR1 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low. The antibodies produced in a cell-free system may be aglycosylated depending on the source of the cells.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxyapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 14. Pharmaceutical Compositions and Methods of Administration

The antibodies or the antibody conjugates provided herein can be formulated into pharmaceutical compositions using methods available in the art and those disclosed herein. Any of the antibodies or the antibody conjugates provided herein can be provided in the appropriate pharmaceutical composition and be administered by a suitable route of administration.

The methods provided herein encompass administering pharmaceutical compositions comprising at least one antibody or antibody conjugate provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions, antibodies, or antibody conjugates provided herein may be administered by any route known in the art. Exemplary routes of administration include, but are not limited to, the inhalation, intraarterial, intradermal, intramuscular, intraperitoneal, intravenous, nasal, parenteral, pulmonary, and subcutaneous routes. In some embodiments, a pharmaceutical composition, antibody, or antibody conjugate provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In some embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibody conjugates.

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a co-solvent. Illustrative examples of co-solvents include ethanol, poly(ethylene)glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene (glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody or antibody conjugate, since, in some embodiments, water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre gelatinized starch, and magnesium stearate.

Also provided are pharmaceutical compositions and dosage forms that comprise one or more excipients that reduce the rate by which an antibody or antibody-conjugate will decompose. Such excipients, which are referred to herein as “stabilizers,” include, but are not limited to, antioxidants such as ascorbic acid, pH buffers, or salt buffers.

### 14.1. Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

### 14.2. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody or antibody conjugate or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram).

In certain embodiment, the dosage of the antibody provided herein, based on weight of the antibody, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose can be administered according to a suitable schedule. In certain embodiments, the dose is administered once, two times, three times, or for times weekly. It may be necessary to use dosages of the antibody or antibody conjugate outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or antibody conjugate or composition provided herein followed by one or more maintenance doses.

In certain embodiments, a dose of an antibody or antibody conjugate or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least 1 day, 2 days, 3 days, 5 days, 10 days, 15 days, 30 days, 45 days, 2 months, 75 days, 3 months, or 6 months.

### 14.3. Combination Therapies and Formulations

In certain embodiments, provided are methods for treatment and/or compositions and therapeutic formulations comprising any of the antibodies or antibody conjugates provided herein in combination with one or more chemotherapeutic agents disclosed herein, and methods of treatment comprising administering such combinations to subjects in need thereof. Examples of chemotherapeutic agents include, but are not limited to, Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially uncialamycin, calicheamicin gammall, and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pladienolide B, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERER (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

In certain embodiments, provided are methods for treatment and/or compositions and therapeutic formulations comprising any of the antibodies or antibody conjugates provided herein in combination with a checkpoint inhibitor, for example, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor.

In certain embodiments, provided are methods for treatment and/or compositions and therapeutic formulations comprising any of the antibodies or antibody conjugates provided herein in combination with one or more PD-1 or PD-L1 inhibitors, and methods of treatment comprising administering such combinations to subjects in need thereof. In some embodiments, the one or more PD-1 or PD-L1 inhibitors comprise a small molecule blocker of the PD-1 or PD-L1 pathway. In some embodiments, the one or more PD-1 or PD-L1 inhibitors comprise an antibody that inhibits PD-1 or PD-L1 activity. In some embodiments, the one or more PD-1 or PD-L1 inhibitors are selected from the group consisting of: CA-170, BMS-8, BMS-202, BMS-936558, CK-301, and AUNP12. In some embodiments, the one or more PD-1 or PD-L1 inhibitors are selected from the group consisting of: avelumab, nivolumab, pembrolizumab, atezolizumab, durvalumab, AMP-224 (GlaxoSmithKline), MEDI0680/AMP-514 (AstraZeneca), PDR001 (Novartis), cemiplimab, TSR-042 (Tesaro), Tizlelizumab/BGB-A317 (Beigene), CK-301 (Checkpoint Therapeutics), BMS-936559 (Bristol-Meyers Squibb), camrelizumab, sintilimab, toripalimab, genolimzumab, and A167 (Sichuan Kelun-Biotech Biopharmaceutical). In some embodiments, the one or more PD-1 or PD-L1 inhibitors are selected from the group consisting of: MGA012 (Incyte/MacroGenics), PF-06801591 (Pfizer/Merck KGaA), LY3300054 (Eli Lilly), FAZ053 (Novartis), PD-11 (Novartis), CX-072 (CytomX), BGB-A333 (Beigene), BI 754091 (Boehringer Ingelheim), JNJ-63723283 (Johnson and Johnson/Jannsen), AGEN2034 (Agenus), CA-327 (Curis), CX-188 (CytomX), STI-A1110 (Servier), JTX-4014 (Jounce), (LLY) AM0001 (Armo Biosciences), CBT-502 (CBT Pharmaceuticals), FS118 (F-Star/Merck KGaA), XmAb20717 (Xencor), XmAb23104 (Xencor), AB122 (Arcus Biosciences), KY1003 (Kymab), RXI-762 (RXi). In some embodiments, the one or more PD-1 or PD-L1 inhibitors are selected from the group consisting of: PRS-332 (Pieris Pharmaceuticals), ALPN-202 (Alpine Immune Science), TSR-075 (Tesaro/Anaptys Bio), MCLA-145 (Merus), MGD013 (Macrogenics), MGD019 (Macrogenics). An additional PD-1 inhibitor is Pidilizumab (CT-011). In some embodiments, the one or more PD-1 or PD-L1 inhibitors are selected from an anti-PD1 mono-specific or bi-specific antibody described in, for example, WO 2016/077397, WO 2018/156777, and International Application No. PCT/US2013/034213, filed May 23, 2018.

In some embodiments, the one or more PD-1 or PD-L1 inhibitors are selected from the group consisting of CA-170, BMS-8, BMS-202, BMS-936558, CK-301, AUNP12, avelumab, nivolumab, pembrolizumab, atezolizumab, durvalumab, AMP-224, MEDI0680/AMP-514, PDR001, cemiplimab, TSR-042, Tizlelizumab/BGB-A317, CK-301, BMS-936559, camrelizumab, sintilimab, toripalimab, genolimzumab, A167, MGA012, PF-06801591, LY3300054, FAZ053, PD-11, CX-072, BGB-A333, BI 754091, JNJ-63723283, AGEN2034, CA-327, CX-188, STI-A1110, JTX-4014, (LLY) AM0001, CBT-502, FS118, XmAb20717, XmAb23104, AB122, KY1003, RXI-762, PRS-33, ALPN-202, TSR-075, MCLA-145, MGD013, and MGD019.

In certain embodiments, provided are methods for treatment and/or compositions and therapeutic formulations comprising any of the antibodies or antibody conjugates provided herein in combination with one or more CTLA-4 inhibitors, and methods of treatment comprising administering such combinations to subjects in need thereof. CTLA-4 inhibitors include, but are not limited to, ipilimumab (Yervoy®), tremelimumab (AstraZeneca and MedImmune), AGEN1884 and AGEN2041 (Agenus).

In certain embodiments, provided are methods for treatment and/or compositions and therapeutic formulations comprising any of the antibodies or antibody conjugates provided herein in combination with one or more LAG-3 inhibitors, and methods of treatment comprising administering such combinations to subjects in need thereof. Examples of LAG-3 immune checkpoint inhibitors include, but are not limited to, BMS-986016 (Bristol-Myers Squibb), GSK2831781 (GaxoSmithKline), IMP321 (Prima BioMed), LAG525 (Novartis), and the dual PD-1 and LAG-3 inhibitor MGD013 (MacroGenics).

In certain embodiments, provided are methods for treatment and/or compositions and therapeutic formulations comprising any of the antibodies or antibody conjugates provided herein in combination with one or more TIM-3 inhibitors, and methods of treatment comprising administering such combinations to subjects in need thereof. A specific TIM-3 inhibitor includes, but is not limited to, TSR-022 (Tesaro).

Other immune checkpoint inhibitors for use in the invention described herein include, but are not limited to, B7-H3/CD276 immune checkpoint inhibitors such as MGA217; indoleamine 2,3-dioxygenase (IDO) immune checkpoint inhibitors such as Indoximod and INCB024360; killer immunoglobulin-like receptors (KIRs) immune checkpoint inhibitors such as Lirilumab (BMS-986015); and, carcinoembryonic antigen cell adhesion molecule (CEACAM) inhibitors (e.g., CEACAM-1, -3 and/or -5).

In certain embodiments, provided are methods for treatment and/or compositions or therapeutic formulations comprising any of the antibody conjugates provided herein in combination with a PARP inhibitor, including, but not limited to olaparib (AZD-2281, Lynparza®), rucaparib (AG 014699, Rubraca®), niraparib (Zejula®), talazoparib (BMN-673, Talzenna®), veliparib (ABT-888), fluzoparib (HS10160), Iniparib (BSI 201), BGB-290, E7016, E7449, and CEP-9722. In one embodiment, an antibody conjugate provided herein is administered in combination with olaparib, rucaparib, niraparib, or talazoparib. In one embodiment, an antibody conjugate provided herein is administered in combination with olaparib.

The agents administered in combination with the antibodies or antibody conjugates disclosed herein can be administered just prior to, sequentially, concurrent with, or shortly after the administration of the antibodies or antibody conjugates. In one embodiment, the agents administered in combination with the antibodies or antibody conjugates disclosed herein are administered sequentially after the administration of the antibody conjugates. In one embodiment, the agents administered in combination with the antibodies or antibody conjugates disclosed herein are administered concurrently after the administration of the antibody conjugates. For purposes of the present disclosure, such administration regimens are considered the administration of an antibody conjugate “in combination with” an additional therapeutically active component. Embodiments include pharmaceutical compositions in which an antibody conjugate disclosed herein is co-formulated with one or more of the chemotherapeutic agents, PD-1 inhibitors, PD-L1 inhibitors, or PARP inhibitors disclosed herein.

### 15. Therapeutic Applications

For therapeutic applications, the antibodies or antibody conjugates provided herein can be administered to a mammal, generally a human, in a pharmaceutically acceptable dosage form such as those known in the art and those discussed above. For example, the antibodies or antibody conjugates may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies or antibody conjugates also are suitably administered by peritumoral, intralesional, or perilesional routes, to exert local as well as systemic therapeutic effects. The intraperitoneal route may be particularly useful, for example, in the treatment of ovarian tumors.

The antibodies or antibody conjugates provided herein may be useful for the treatment of any disease or condition involving receptor ROR1. In some embodiments, the disease or condition is a disease or condition that can be diagnosed by overexpression of ROR1. In some embodiments, the disease or condition is a disease or condition that can benefit from treatment with an anti-ROR1 antibody. In some embodiments, the disease or condition is a cancer.

Any suitable cancer may be treated with the antibodies or antibody conjugates provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer (including triple-negative breast cancer, or TNBC), bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fallopian tube carcinoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, primary peritoneal carcinoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In some embodiments, the disease to be treated with the antibodies or antibody conjugates provided herein is gastric cancer, colorectal cancer, renal cell carcinoma, cervical cancer, non-small cell lung carcinoma, ovarian cancer, uterine cancer, fallopian tube carcinoma, primary peritoneal carcinoma, uterine corpus carcinoma, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In some embodiments, the disease is ovarian cancer. In some embodiments, the disease is breast cancer. In some embodiments, the disease is triple-negative breast cancer (TNBC). In some embodiments, the disease is PARP inhibitor-resistant triple-negative breast cancer (TNBC). In some embodiments, the disease is lung cancer. In some embodiments, the disease is non-small cell lung cancer (NSCLC). In some embodiments, the disease is head and neck cancer. In some embodiments, the disease is renal cell carcinoma. In some embodiments, the disease is brain carcinoma. In some embodiments, the disease is endometrial cancer.

In certain embodiments, the cancer is selected from ovarian cancer, endometrial cancer, breast cancer, triple negative breast cancer, colorectal cancer, prostate cancer, gastric cancer, non-Hodgkin's lymphoma, melanoma, renal cancer, and pancreatic cancer. In certain embodiments, the cancer is a hematological cancer. In certain embodiments, the cancer is selected from lymphomas, leukemias, and myelomas. In certain embodiments, the cancer is selected from CLL, MCL, DLBCL, and ALL.

In certain embodiments, the antibodies or antibody conjugates provided herein treat the disease, including cancer, by inducing immunogenic cell death. In certain embodiments, cells treated with an antibody or antibody conjugate provided herein are characterized by cell-surface calreticulin and/or by release of HMGB1. In certain embodiments, cells treated with an antibody or an antibody conjugate provided herein elicited monocyte activation.

In certain embodiments, the antibodies or antibody conjugates provided herein treat the disease, for example cancer, by activating anti-tumor immunity or protective immunity. In certain embodiments, tumor cells pre-treated with an antibody or antibody conjugate provided herein undergo immunogenic cell damage, which can, in turn mount protective immunity in vivo.

In other embodiments, the antibodies or antibody conjugates provided herein in combination with a checkpoint inhibitor described herein treat the disease, for example cancer, by activating anti-tumor immunity or protective immunity. In certain embodiments, tumor cells pre-treated with an antibody or antibody conjugate provided herein undergo immunogenic cell damage, which can, in turn mount protective immunity in vivo. In certain embodiments, the checkpoint inhibitor is a PD-1 or PD-L1 inhibitor.

### 16. Diagnostic Applications

In some embodiments, the antibody or antibody conjugates provided herein are used in diagnostic applications. For example, an anti-ROR1 antibody or antibody conjugate may be useful in assays for ROR1 protein. In some aspects the antibody or antibody conjugate can be used to detect the expression of ROR1 in various cells and tissues. These assays may be useful, for example, in making a diagnosis and/or prognosis for a disease, such as a cancer.

In some diagnostic and prognostic applications, the antibody or antibody conjugate may be labeled with a detectable moiety. Suitable detectable moieties include, but are not limited to radioisotopes, fluorescent labels, and enzyme-substrate labels. In another embodiment, the anti-ROR1 antibody or antibody conjugate need not be labeled, and the presence of the antibody or antibody conjugate can be detected using a labeled antibody which specifically binds to the anti-ROR1 antibody or antibody conjugate.

Also provided herein is a method of diagnosing cancer in a subject in need thereof, comprising:
a) administering to the subject an effective amount of an antibody conjugate described herein, an antibody described herein, or a pharmaceutical composition described herein, wherein the antibody drug conjugate or antibody optionally comprises a label; and
b) detecting the antibody drug conjugate or the antibody or detecting the label comprised in the antibody drug conjugate or antibody.

Also provided herein is a method of diagnosing cancer in a subject in need thereof, comprising detecting the expression of ROR1 in a cell or tissue wherein the method comprises:
a) administering to the subject an effective amount of an antibody conjugate described herein, an antibody described herein, or a pharmaceutical composition described herein, wherein the antibody drug conjugate or antibody optionally comprises a label; and
b) detecting the antibody drug conjugate or antibody or detecting the label comprised in the antibody drug conjugate or antibody.

Also provided herein is a method of diagnosing cancer in a subject in need thereof, comprising:
a) detecting the expression of ROR1 in a cell or tissue of the subject; and
b) administering to the subject an effective amount of an antibody conjugate described herein, an antibody described herein, or a pharmaceutical composition described herein.

### 17. Affinity Purification Reagents

The antibodies or antibody conjugates provided herein may be used as affinity purification agents. In this process, the antibodies or antibody conjugates may be immobilized on a solid phase such a resin or filter paper, using methods well known in the art. The immobilized antibodies or antibody conjugate is contacted with a sample containing the ROR1 protein (or fragment thereof) to be purified, and thereafter the support is washed with a suitable solvent that will remove substantially all the material in the sample except the ROR1 protein, which is bound to the immobilized antibody. Finally, the support is washed with another suitable solvent, such as glycine buffer, pH 5.0 that will release the ROR1 protein from the antibody.

### 18. Kits

In some embodiments, an anti-ROR1 antibodies or antibody conjugate provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the anti-ROR1 antibodies or antibody conjugate. In some embodiments, the anti-ROR1 antibodies or antibody conjugate is provided in the form of a pharmaceutical composition.

What is claimed is:

1. An antibody conjugate comprising an antibody that specifically binds to receptor tyrosine kinase orphan receptor 1 (ROR1) linked site-specifically to at least one payload moiety, wherein the antibody comprises one or more non-natural amino acids.

2. The antibody conjugate of claim 1, wherein the one or more non-natural amino acids is at an antibody amino acid site other than the amino terminus of the antibody.

3. The antibody conjugate of claim 1, wherein the one or more non-natural amino acids is at an antibody amino acid site other than the carboxy terminus of the antibody.

4. The antibody conjugate of any one of claims 1-3, wherein the antibody comprises one or more non-natural amino acids selected from the group consisting of: HC F404, HC-K121, HC-Y180, HC-F241, HC-221, LC-T22, LC-S7, LC-N152, LC-K42, LC-E161, LC-D170, HC-S136, HC S25, HC-A40, HC-S119, HC-S190, HC-K222, HC-R¹⁹, HC-Y52, and HC-S70, according to the Kabat, Chothia, or EU numbering scheme.

5. The antibody conjugate of claim 4, wherein the antibody comprises one or more non-natural amino acids selected from the group consisting of: HC-F404, HC-Y180, HC-F241, LC-K42, and LC-E161, according to the Kabat, Chothia, or EU numbering scheme.

6. The antibody conjugate of claim 4, wherein the antibody comprises one or more non-natural amino acids at sites selected from the group consisting of: HC-F404, HC-Y180, LC-K42, and LC-E161, according to the Kabat or EU numbering scheme of Kabat.

7. The antibody conjugate of claim 4, wherein the antibody comprises one or more non-natural amino acids at sites selected from the group consisting of: HC-F404, HC-Y180, HC-F241, and LC-K42, according to the Kabat or EU numbering scheme of Kabat.

8. The antibody conjugate of any one of the preceding claims, wherein a residue of the one or more non-natural amino acids is linked to the payload moiety via a linker that is hydrolytically stable.

9. The antibody conjugate of any one of the preceding claims, wherein a residue of the one or more non-natural amino acids is linked to the payload moiety via a linker that is cleavable.

10. The antibody conjugate of any one of the preceding claims, wherein the one or more non-natural amino acids is selected from the group consisting of p-acetyl-L-phenylalanine, O-methyl-L-tyrosine, an -3-(2-naphthyl) alanine, 3-methyl-phenylalanine, O-4-allyl-L-tyrosine, 4-propyl-L-tyrosine, a tri O-acetyl-GlcNAcβ-serine, L-Dopa, fluorinated phenylalanine, isopropyl-L-phenylalanine, p-azido-L-phenylalanine, p-azido-methyl-L-phenylalanine, compound 56, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, isopropyl-L-phenylalanine, and p-propargyloxy-phenylalanine.

11. The antibody conjugate of any one of the preceding claims, wherein the residue of the one or more non-natural amino acids is a residue of compound (30) or compound (56).

12. The antibody conjugate of any one of the preceding claims, wherein the one or more non-natural amino acids is p-azidomethylphenylalanine.

13. The antibody conjugate of any one of the preceding claims, wherein the one or more non-natural amino acids is p-azido-methyl-L-phenylalanine.

14. The antibody conjugate of any one of the preceding claims wherein the payload moiety is selected from the group consisting of maytansines, hemiasterlins, amanitins, camptothecans, exatecans, anthracyclines, pyrrolobenzodiazepines, and auristatins.

15. The antibody conjugate of any one of the preceding claims wherein the payload moiety is selected from the group consisting of DM1, hemiasterlin, amanitin, camptothecan, exatecan, exatecan derivative (Dxd), SN-38, PNU-159862, PNU derivative (PNU-EDA), pyrrolobenzodiazepine, MMAF, and MMAE.

16. The antibody conjugate of any one of the preceding claims, according to the structure of Formula I: 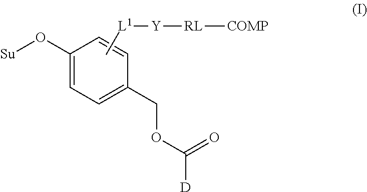
or a pharmaceutically acceptable salt thereof, wherein
COMP is a residue of the anti-ROR1 antibody;
L¹ is —C₁₋₆ alkylene-;
Y is —X¹—C₁₋₆ alkylene-[X¹—C₁₋₆ alkylene]ₙ-[X¹]ₚ—, —X¹—C₂₋₆ alkenylene-[X¹—C₂₋₆ alkenylene]ₙ-[X¹]ₚ—, —X¹—C₂₋₆ alkynylene-[X¹—C₂₋₆ alkynylene]ₙ-[X¹]ₚ—, wherein at least one alkylene, alkenylene or alkynylene in Y is substituted with one or more substituents selected from R⁵⁰; and
wherein the alkylene, alkenylene, or alkynylene in Y is optionally substituted with one or more substituents selected from R⁵¹;
R⁵⁰ is —C₁₋₆ alkylene-X²—[C₁₋₆ alkylene]ₘ-POLY,
—C₂₋₆ alkenylene-X²—[C₂₋₆ alkenylene]ₘ-POLY, or —C₂₋₆ alkynylene-X²—[C₂₋₆ alkynylene]ₘ-POLY, wherein each alkylene, alkenylene or alkynylene of R⁵⁰ is optionally substituted with one or more substituents selected from halogen, —CN, —NO₂, —OH, —N(R¹⁰)₂, —C(O)N(R¹⁰)₂,
—C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
R⁵¹ is independently selected from halogen, —CN, —NO₂, —OH, —N(R¹⁰)₂, —C(O)N(R¹⁰)₂, —C(O)—, —C(S)—, —C(O)OCH₂C₆H₅,
—NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
X¹ and X² are independently selected from —C(O)— and —N(R¹⁰)C(O)—;
R¹⁰ is independently selected at each occurrence from hydrogen, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
POLY is a water-soluble polymer;
n is an integer selected from zero, one, two, and three;
m is an integer selected from zero and one;
p is an integer selected from zero and one;
Su is a hexose form of a monosaccharide;
D is a drug moiety; and
RL is a reactive group residue.

17. The antibody conjugate of any one of the preceding claims, according to the structure of Formula II: 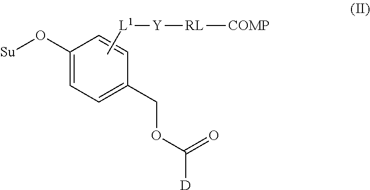
or a pharmaceutically acceptable salt thereof, wherein COMP is a residue of the ROR1 antibody;
L¹ is —C₁₋₆ alkylene-;
Y is —X¹—C₁₋₆ alkylene-[X¹—C₁₋₆ alkylene]ₙ-X¹—, —X¹—C₂₋₆ alkenylene-[X¹—C₂₋₆ alkenylene]ₙ-X¹—, —X¹—C₂₋₆ alkynylene-[X¹—C₂₋₆ alkynylene]ₙ-X¹, wherein at least one alkylene, alkenylene or alkynylene in Y is substituted with one or more substituents selected from R⁵⁰;
R⁵⁰ is —C₁₋₆ alkylene-X²—[C₁₋₆ alkylene]ₘ-POLY, —C₂₋₆ alkenylene-X²—[C₂₋₆ alkenylene]ₘ-POLY, or —C₂₋₆ alkynylene-X²—[C₂₋₆ alkynylene]ₘ-POLY, wherein each alkylene, alkenylene or alkynylene of R⁵⁰ is optionally substituted with one or more substituents selected from halogen, —CN, —NO₂, —OH, —N(R¹⁰)₂, —C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
X¹ and X² are independently selected from —C(O)— and —N(R¹⁰)C(O)—;
R¹⁰ is independently selected at each occurrence from hydrogen, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl;
POLY is a water-soluble polymer;
n is an integer selected from zero, one, two, and three;
m is an integer selected from zero and one;
Su is a hexose form of a monosaccharide;
D is a drug moiety; and
RL is a reactive group residue.

18. The conjugate of claim 17, wherein the compound of Formula (II) is according to Formula (IIA)

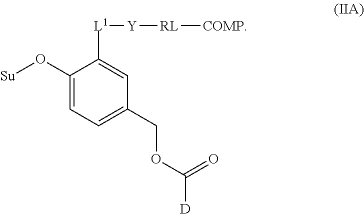

19. The conjugate of any one of claims 16 to 18, wherein Su is 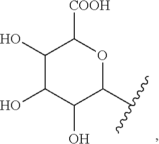 [SMILES: *C1OC(C(=O)O)C(O)C(O)C1O]
wherein 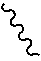
represents attachment to the remainder of the compound.

20. The conjugate of any one of claims 16 to 19, wherein Su is 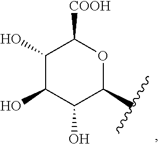 [SMILES: *[C@@H]1O[C@H](C(=O)O)[C@@H](O)[C@H](O)[C@H]1O]
wherein 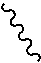
represents attachment to the remainder of the compound.

21. The conjugate of any one of claims 16 to 20, wherein D is a cytotoxic payload.

22. The conjugate of claim 21, wherein the cytotoxic payload is a tubulin inhibitor, a DNA topoisomerase I inhibitor, or a DNA topoisomerase II inhibitor.

23. The conjugate of any one of claims 16 to 20, wherein D is selected from the group consisting of hemiasterlins, camptothecins, anthracyclines, PNU-159682, and EDA PNU-159682 derivatives.

24. The conjugate of any one of claims 16 to 20, wherein D is hemiasterlin, exatecan, PNU-159682, or EDA PNU-159682 derivatives.

25. The conjugate of any one of claims 16 to 24, wherein L¹ is —C₁₋₃ alkylene-.

26. The conjugate of any one of claims 16 to 25, wherein Y is
—X¹—C₁₋₆ alkylene-[X¹—C₁₋₆ alkylene]ₙ-X¹-, wherein at least one alkylene in Y is substituted with one or more substituents selected from R⁵⁰.

27. The conjugate of any one of claims 16 to 26, wherein Y is
—X¹—C₁₋₄ alkylene-X¹—C₁₋₄ alkylene-X¹—C₁₋₄ alkylene-X¹—, wherein at least one alkylene in Y is substituted with one or more substituents selected from R⁵⁰.

28. The conjugate of any one of claims 16 to 26, wherein Y is —X¹—C₁₋₄ alkylene-X¹—C₁₋₄ alkylene-X¹—, wherein at least one alkylene in Y is substituted with one or more substituents selected from R⁵⁰.

29. The conjugate of any one of claims 16 to 28, wherein R⁵⁰ is
—C₁₋₆ alkylene-X²—[C₁₋₆ alkylene]ₘ-POLY, wherein each alkylene of R⁵⁰ is optionally substituted with one or more substituents selected from halogen, —CN, —NO₂, —OH, —N(R¹⁰)₂,
—C(O)N(R¹⁰)₂, —C(O)—, —C(S)—, —C(O)OCH₂C₆H₅, —NHC(O)OCH₂C₆H₅, C₁₋₁₀ alkyl, C₂₋₁₀ alkenyl, C₂₋₁₀ alkynyl, C₃₋₁₂ carbocycle, 3- to 12-membered heterocycle, and C₁₋₁₀ haloalkyl.

30. The conjugate of any one of claims 16 to 29, wherein m is one.

31. The conjugate of any one of claims 16 to 30, wherein POLY is polyethylene glycol (PEG), methoxypolyethylene glycol (mPEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), polysarcosine, or a combination thereof.

32. The conjugate of any one of claims 16 to 31, wherein POLY comprises a polyethylene glycol (PEG) or methoxypolyethylene glycol (mPEG).

33. The conjugate of any one of claims 16 to 32, wherein RL comprises an alkyne, cyclooctyne, a strained alkene, a tetrazine, a thiol, a para-acetyl-phenylalanine residue, an oxyamine, amine, a maleimide, or an azide.

34. The conjugate of any one of claims 16 to 33, wherein RL is selected from the group consisting of

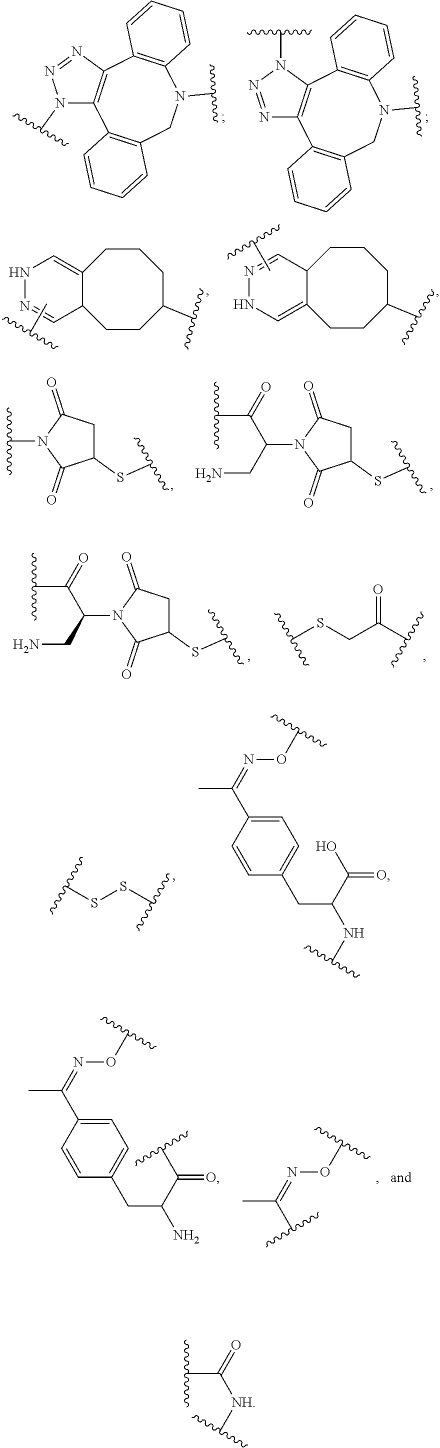

35. The conjugate of any of claim 1-15, according to Formula III: 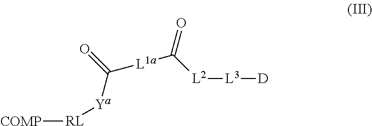
or a pharmaceutically acceptable salt thereof;
wherein
L^{1a} is selected from 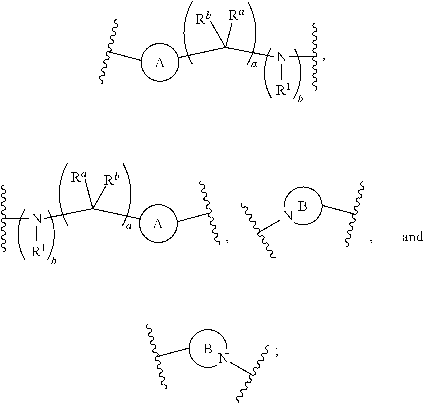
Ring A is an optionally substituted bridged, fused, or spirocyclic bicyclic carbocycle, or an optionally substituted bridged, fused, or spirocyclic bicyclic heterocycle, wherein the carbocycle or the heterocycle of Ring A are optionally substituted with one or more substituents selected from alkyl, alkenyl, alkynyl, cycloalkyl, halogen, alkoxy, —CN, —NO₂, —OH, —N(R²R³)₂, —C(O)—,
—C(O)N(R²R³)₂, —C(O)OR², aminoalkyl, hydroxyalkyl, haloalkyl, aryl, arylalkyl, heteroaryl, and heteroarylalkyl;
Ring B is an optionally substituted N-linked bridged, fused, or spirocyclic bicyclic heterocycle, wherein Ring B is optionally substituted with one or more substituents selected from alkyl, alkenyl, alkynyl, cycloalkyl, halogen, alkoxy, —CN, —NO₂, —OH, —N(R²R³)₂, —C(O)—,
—C(O)N(R²R³)₂, —C(O)OR², aminoalkyl, hydroxyalkyl, haloalkyl, aryl, arylalkyl, heteroaryl, and heteroarylalkyl;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
b is an integer selected from 0 or 1;
R¹ is hydrogen or alkyl optionally substituted with one or more substituents selected from cycloalkyl, halogen, alkoxy, —CN, —NO₂, —OH, —N(R²R³)₂, —C(O)N(R²R³)₂, —C(O)OR², aryl, and heteroaryl;
R² and R³ are independently selected from hydrogen, alkyl, cycloalkyl, aryl, arylalkyl, heteroaryl, and heteroarylalkyl;
Y^{a} is *—C(O)—(CR^{a}R^{b})_{c}—NH— or *—C(O)—(CR^{a}R^{b})_{c}—, wherein * represents where Y^{a} is bound to RL;
c is an integer selected from 1, 2, 3, 4, 5, or 6;
RL is a reactive group residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ-C(O)—Z-L⁴-OC(O)—, —Z-AA-, -AA-, —C(O)—, —C(O)-AA-Z—(CR^{a}R^{b})ₐ—,
-AA-C(O)—, —C(O)—(CR^{a}R^{b})ₐ—Z—(CR^{a}R^{b})—Z-AA-C(O)—, —C(O)O-LA-Z—C(O)—(CR^{a}R^{b})ₐ—Z—C(O)—, -AA-Z-; and —(CR^{a}R^{b})ₐ—Z-AA-C(O)—;
Z is selected from —NR²— and —O—;
AA is an amino acid residue or a peptide residue;
L⁴ is 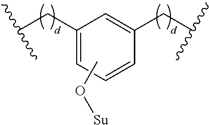
wherein Su is a hexose form of a monosaccharide;
d is an integer independently selected from 1, 2, and 3;
D is a drug moiety;
COMP is a residue of the ROR1 antibody; and 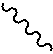
represents attachment to the remainder of the compound.

36. The conjugate of any one of claims 1-15, of Formula (V): 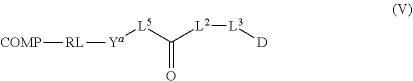
or a pharmaceutically acceptable salt and/or regioisomer thereof;
wherein
L⁵ is a linker comprising an unnatural amino acid;
Y^{a} is *—C(O)—(CR^{a}R^{b})_{c}—NH— or *—C(O)—(CR^{a}R^{b})_{c}—, wherein * represents where Y^{a} is bound to RL;
c is an integer selected from 1, 2, 3, 4, 5, or 6;
RL is a reactive group residue;
L² is absent or a linker comprising a hydrophilic polymer residue;
L³ is absent, —C(O)-AA-, —C(O)-AA-Z—(CR^{a}R^{b})ₐ—Z—(CR^{a}R^{b})ₐ—C(O)—, —C(O)—Z—(CR^{a}R^{b})ₐ—C(O)—Z-LA-OC(O)—, —Z-AA-, -AA-, —C(O)—, —C(O)-AA-Z—(CR^{a}R^{b})ₐ—, -AA-C(O)—, —C(O)—(CR^{a}R^{b})ₐ-Z—(CR^{a}R^{b})-Z-AA-C(O)—, —C(O)O-L⁴-Z—C(O)—(CR^{a}R^{b})ₐ-Z—C(O)—, -AA-Z-; and —(CR^{a}R^{b})ₐ-Z-AA-C(O)—;
R^{a} and R^{b} are independently selected from hydrogen, alkyl, alkenyl, alkynyl, cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl;
a is an integer independently selected from 0, 1, 2, 3, 4, 5, and 6;
Z is selected from —NR²— and —O—;
AA is an amino acid residue or a peptide residue;
L⁴ is 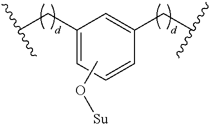
wherein Su is a hexose form of a monosaccharide;
d is an integer independently selected from 1, 2, and 3;
D is a drug moiety;
COMP is a residue of the ROR1 antibody; and 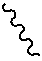
represents attachment to the remainder of the compound.

37. The conjugate of claim 35, wherein Ring B is selected from

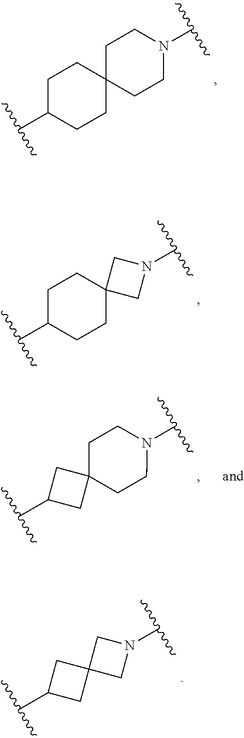 [SMILES: *C1CCC2(CC1)CCN(*)CC2 ; *C1CCC2(CC1)CN(*)C2 ; *C1CC2(CCN(*)CC2)C1 ; *C1CC2(C1)CN(*)C2]

38. The conjugate of claim 35, where Ring B is selected from 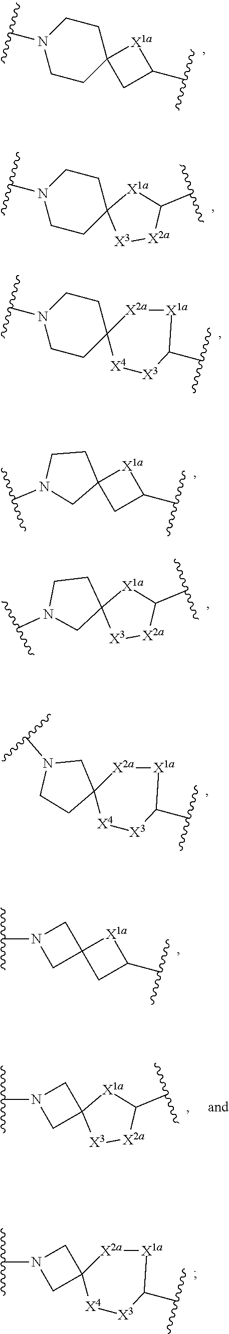
wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}-X³ is —C(R⁴)₂— and when X^{1a}, X^{2a}, X³, and X⁴ are present, at least two of X^{1a}-X⁴ are —C(R⁴)₂; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

39. The conjugate of claim 35, wherein Ring B is selected from

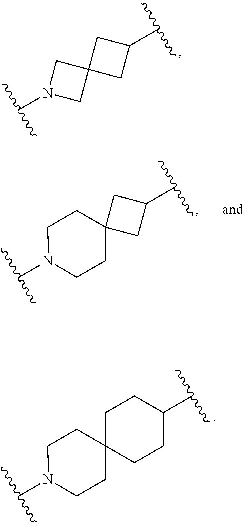 [SMILES: *C1CC2(C1)CN(*)C2 ; *C1CC2(CCN(*)CC2)C1 ; *C1CCC2(CC1)CCN(*)CC2]

40. The conjugate of claim 35, wherein Ring A is selected from 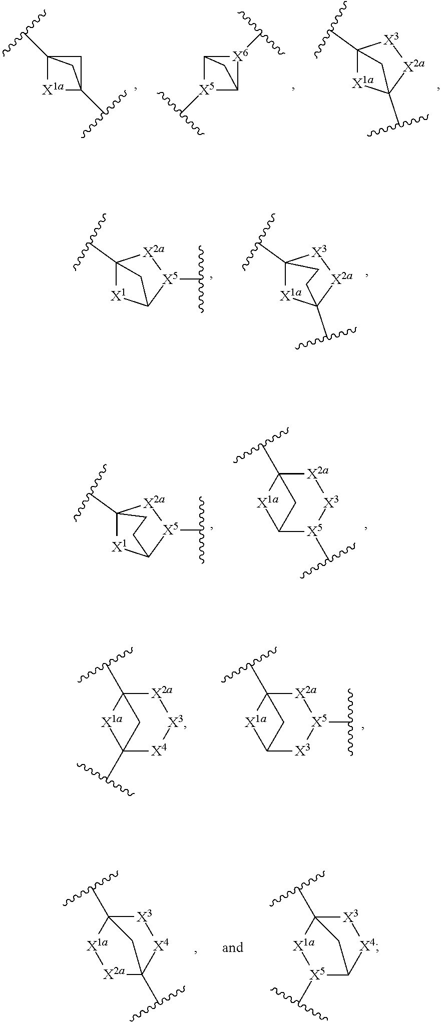
wherein X^{1a}, X^{2a}, X³, and X⁴ are independently selected from —C(R⁴)₂—, —NH—, —O—, and —S— wherein when X^{1a}, X^{2a}, and X³ are present, at least one of X^{1a}-X³ is —C(R⁴)₂— and when X^{1a}, X^{2a}, X³, and X⁴ are present, at least two of X^{1a}-X⁴ are —C(R⁴)₂;
X⁵ is N or CR⁴; and
R⁴ is independently selected from hydrogen, C₁₋₆ alkyl, C₂₋₆ alkenyl, C₂₋₆ alkynyl, C₃₋₁₂ cycloalkyl, halogen, alkoxy, —CN, —NO₂, —OH, —N(R²R³)₂, —C(O)N(R²R³)₂, —C(O)OR², aminoalkyl, hydroxyalkyl, haloalkyl, aryl, arylalkyl, heteroaryl, and heteroarylalkyl; or two R⁴ groups on the same carbon are taken together to form an oxo group.

41. The conjugate any one of claims 35 and 37-40, wherein L^{1a} is

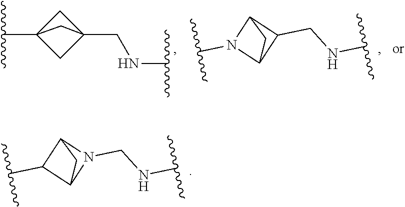

42. The conjugate of any one of claims 35-41, wherein L² is —(CR^{a}R^{b})ₐ-POLY¹-, 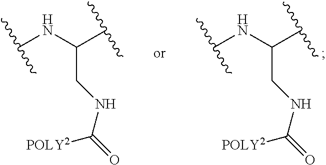
wherein POLY¹- is a divalent residue of polyethylene glycol (PEG), poly(propylene glycol) (PPG), or a copolymer of ethylene glycol and propylene glycol; wherein 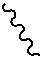
represents an attachment to the remainder of the compound.

43. The conjugate of any one of claims 35-42, wherein L³ is —C(O)-AA-, —C(O)-AA-Z—(CR^{a}R^{b})ₐ—Z—(CR^{a}R^{b})ₐ—C(O)—, —Z-AA-, or -AA-.

44. The conjugate of any one of claims 35-43, wherein Su is

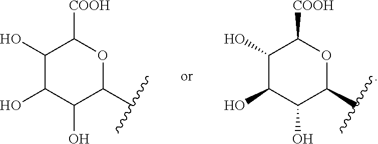 [SMILES: *C1OC(C(=O)O)C(O)C(O)C1O ; *[C@@H]1O[C@H](C(=O)O)[C@@H](O)[C@H](O)[C@H]1O]

45. The conjugate of any one of claims 35-44, wherein the D is a drug moiety selected from a tubulin inhibitor, DNA damaging agents, a DNA topoisomerase I inhibitor, or a DNA topoisomerase II inhibitor, RNA polymerase II inhibitor, a hemiasterlin residue, a camptothecin residue, camptothexin derivatives, an anthracycline residue, PNU-159682 and PNU-159682 derivative, PBDs, duocarmycins.

46. The antibody conjugate of any one of the previous claims, wherein the antibody conjugate has a structure selected from the group consisting of: 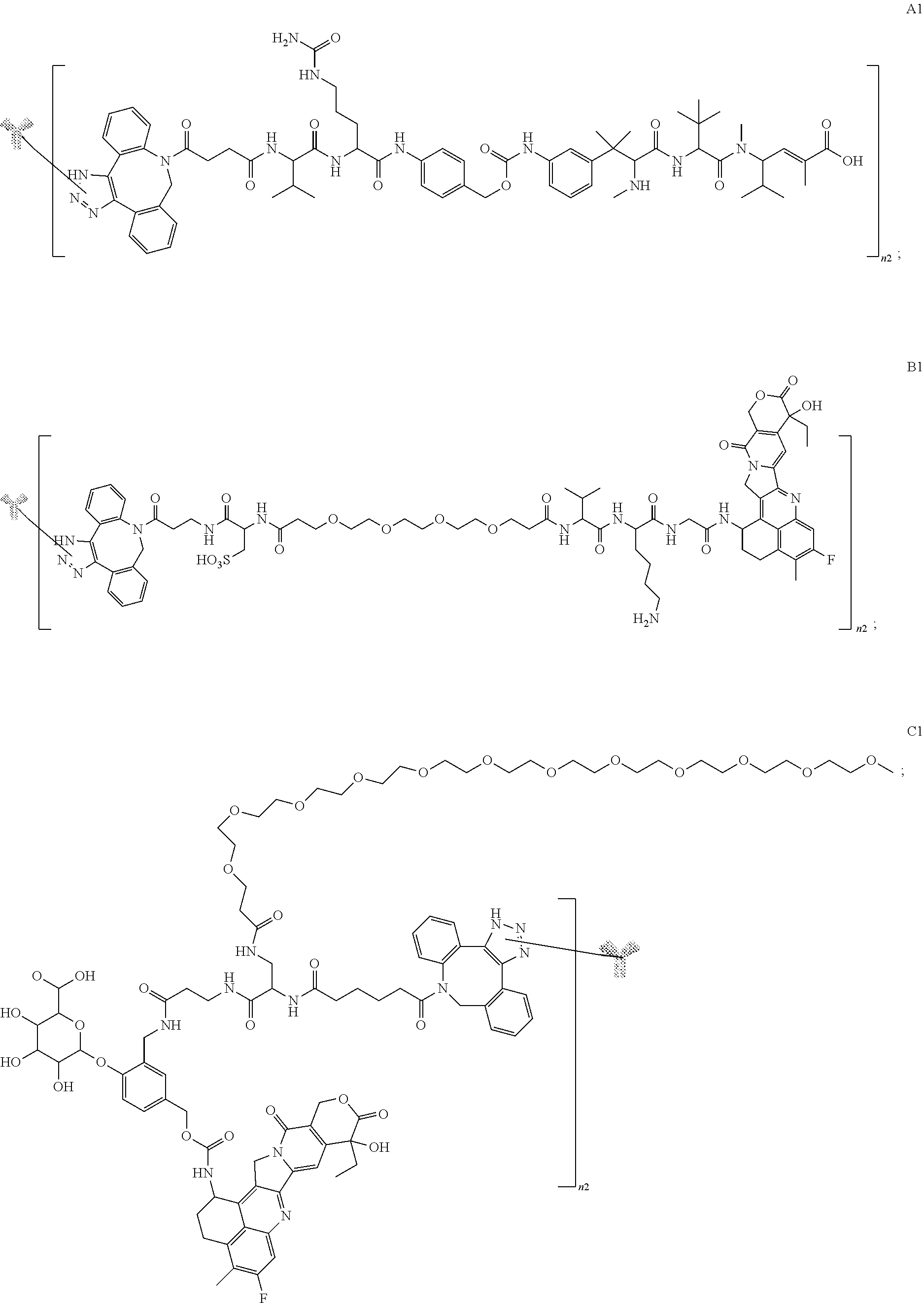 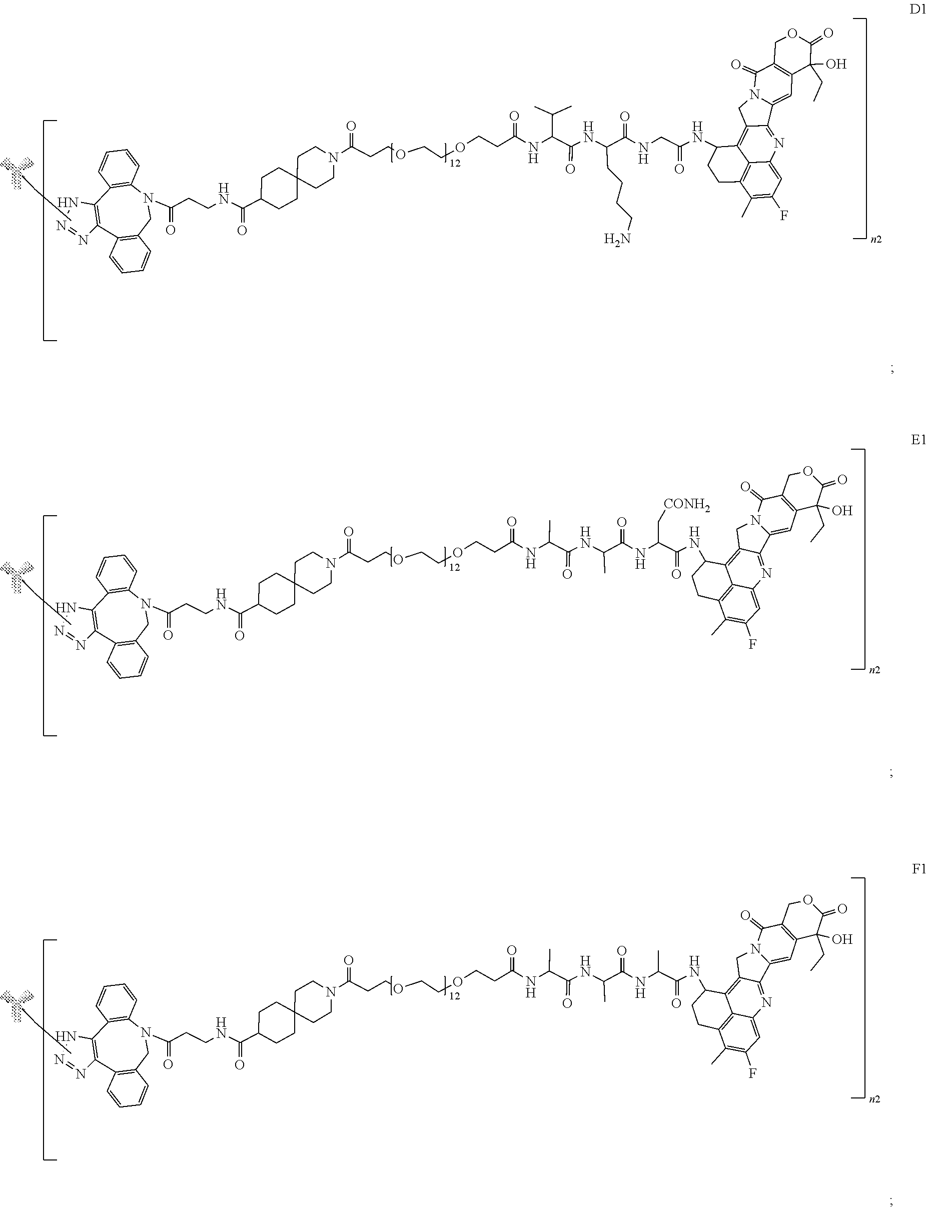 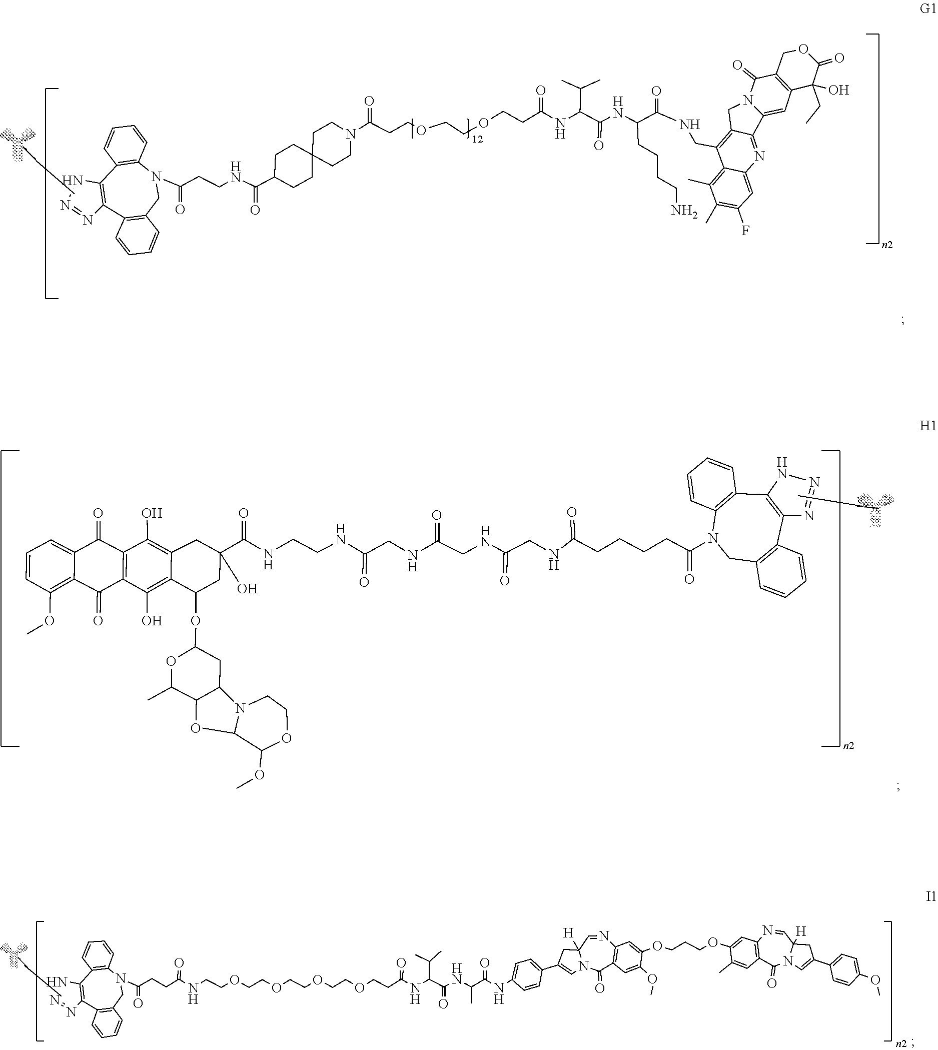 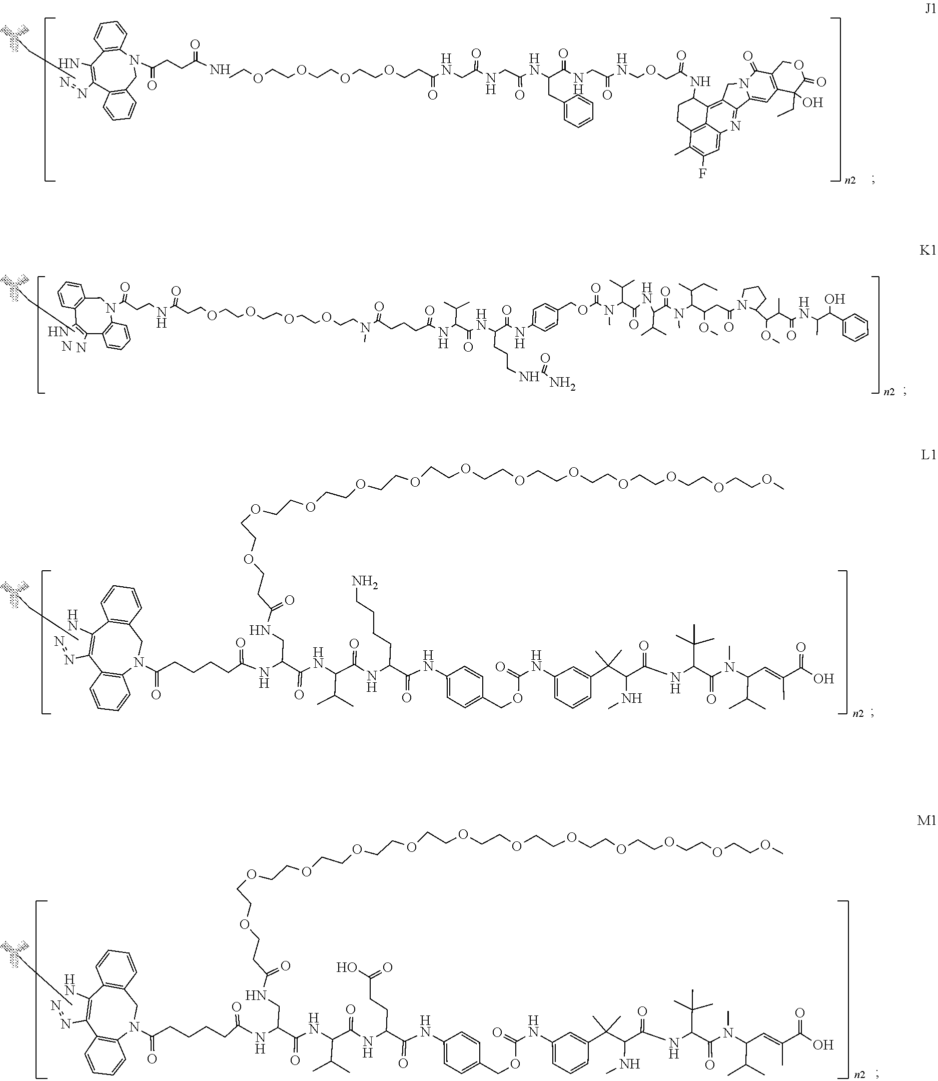 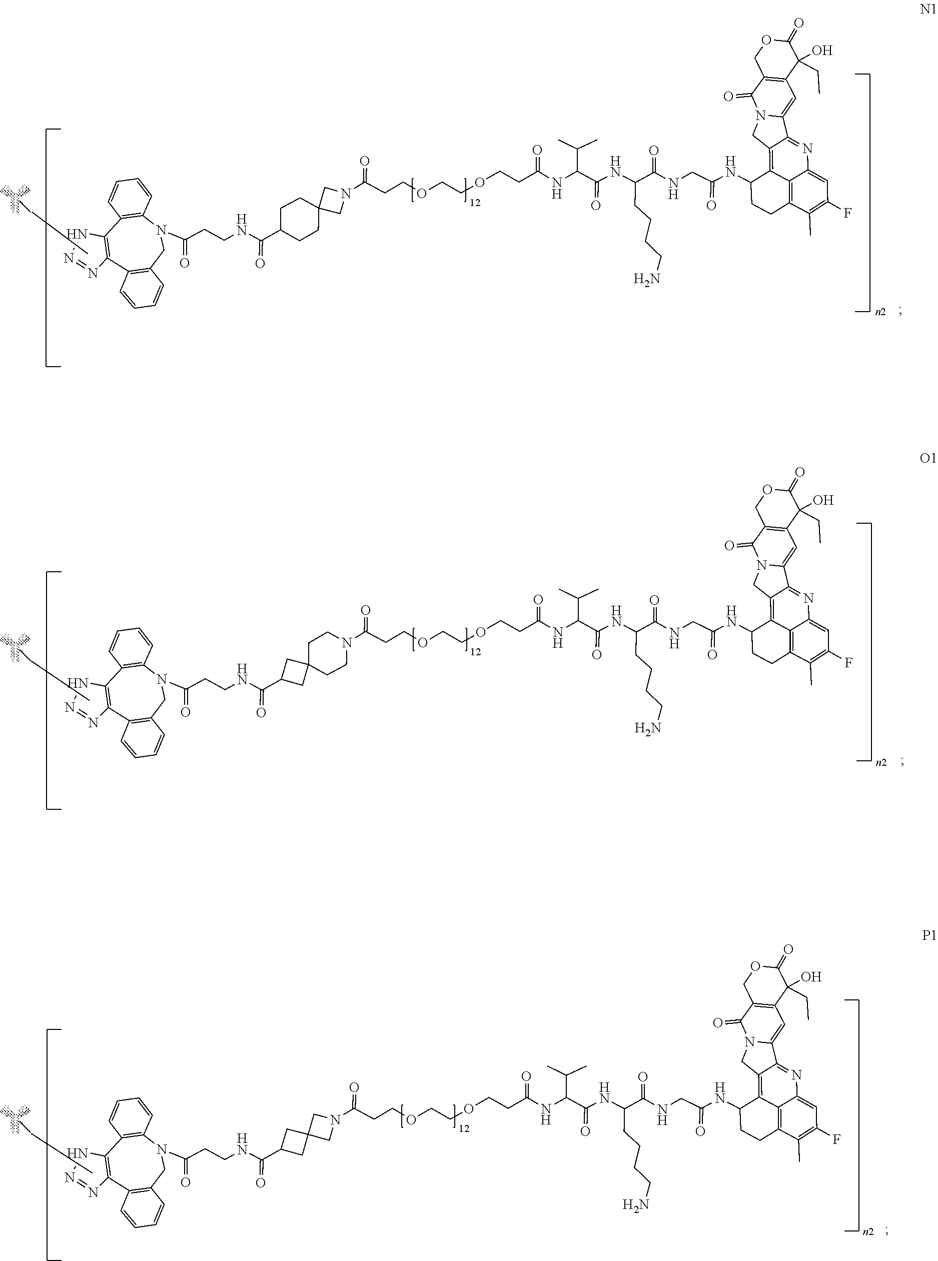 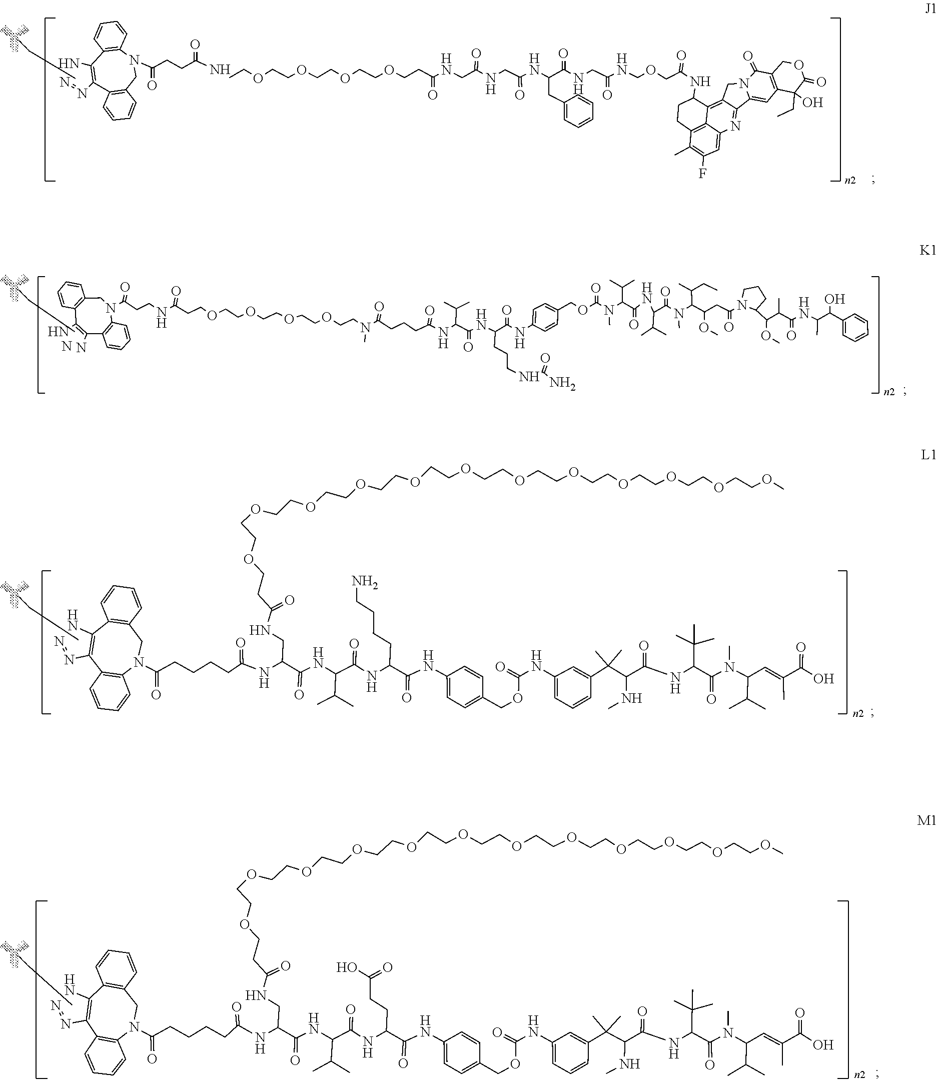 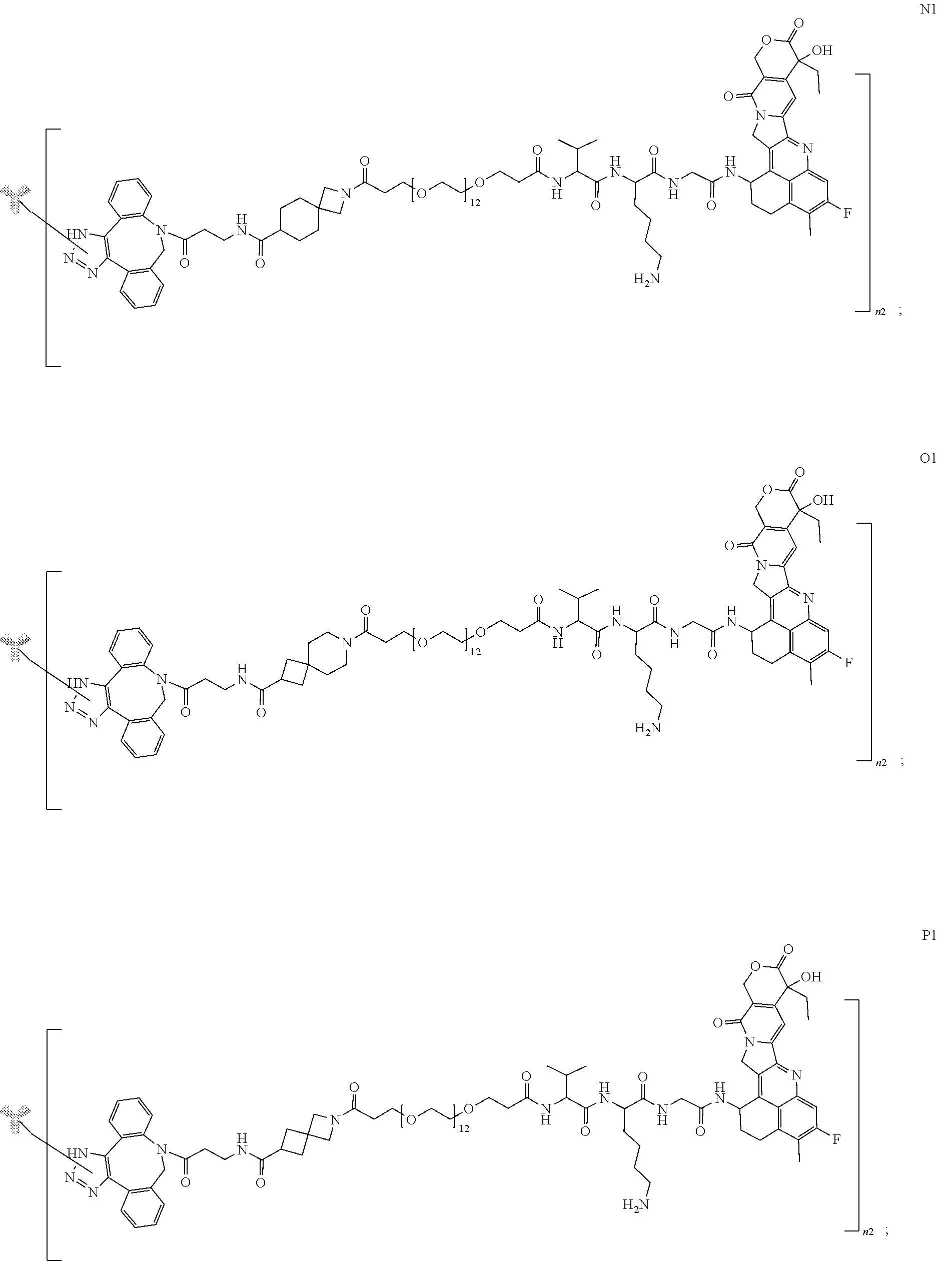 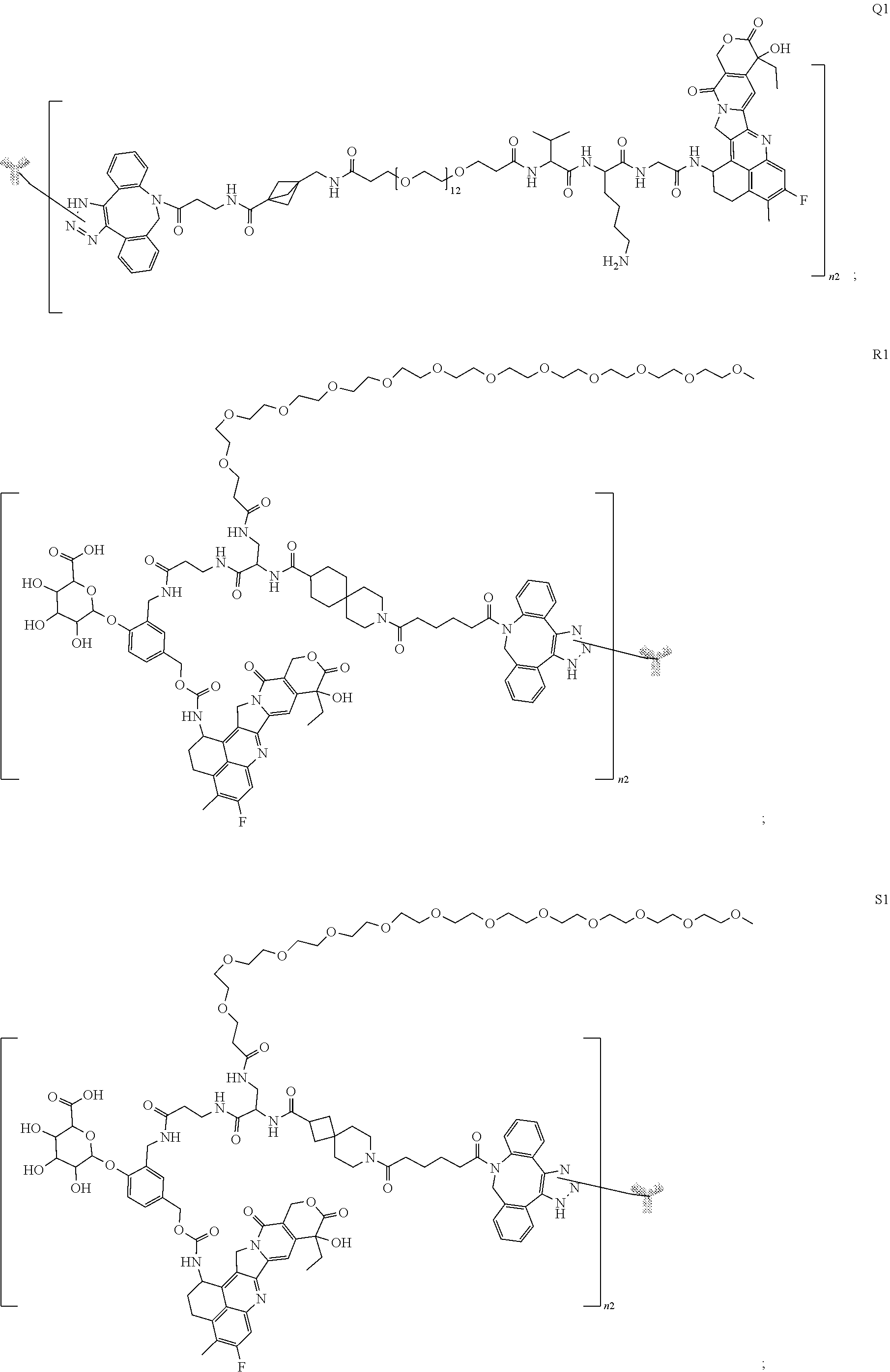 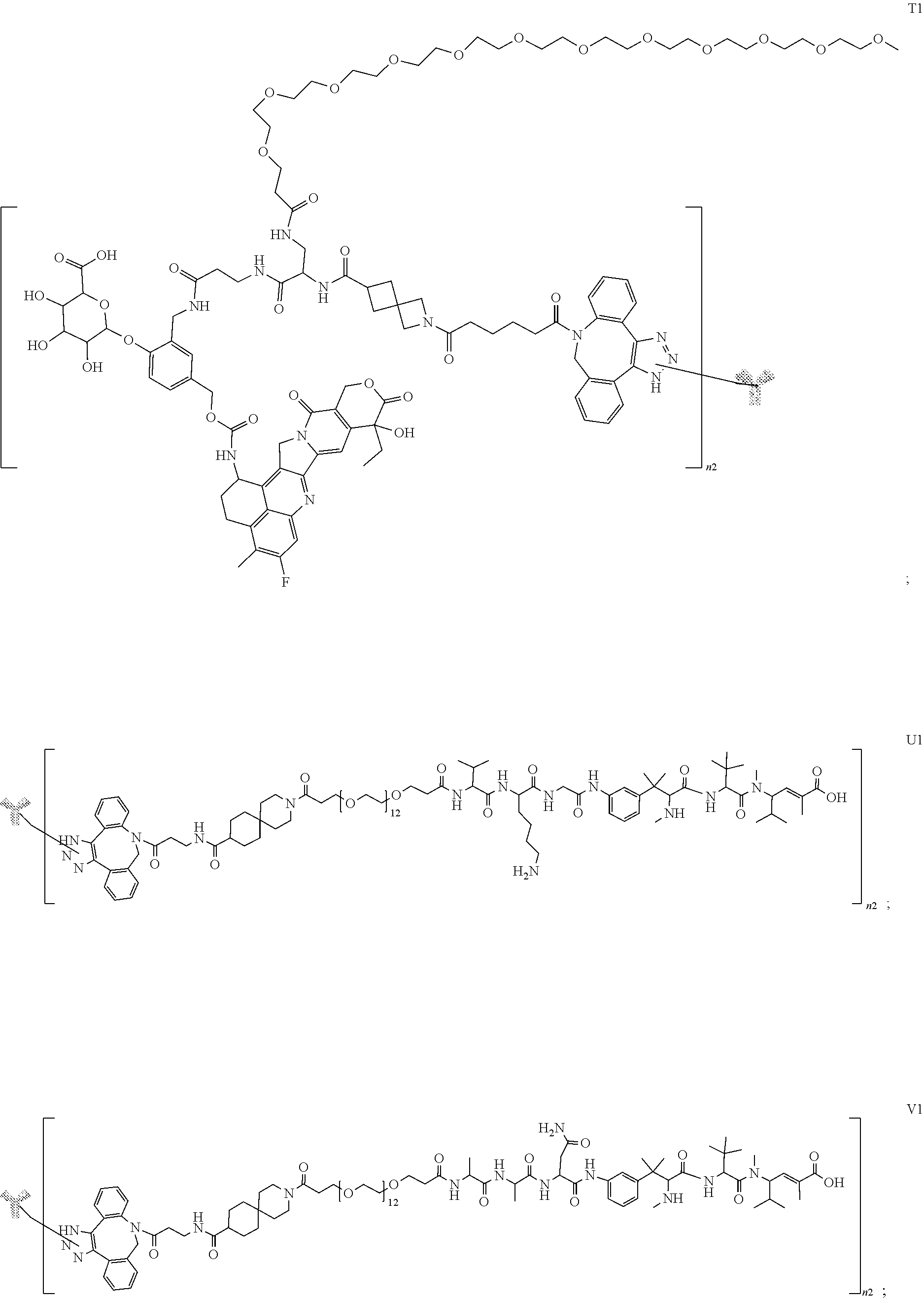 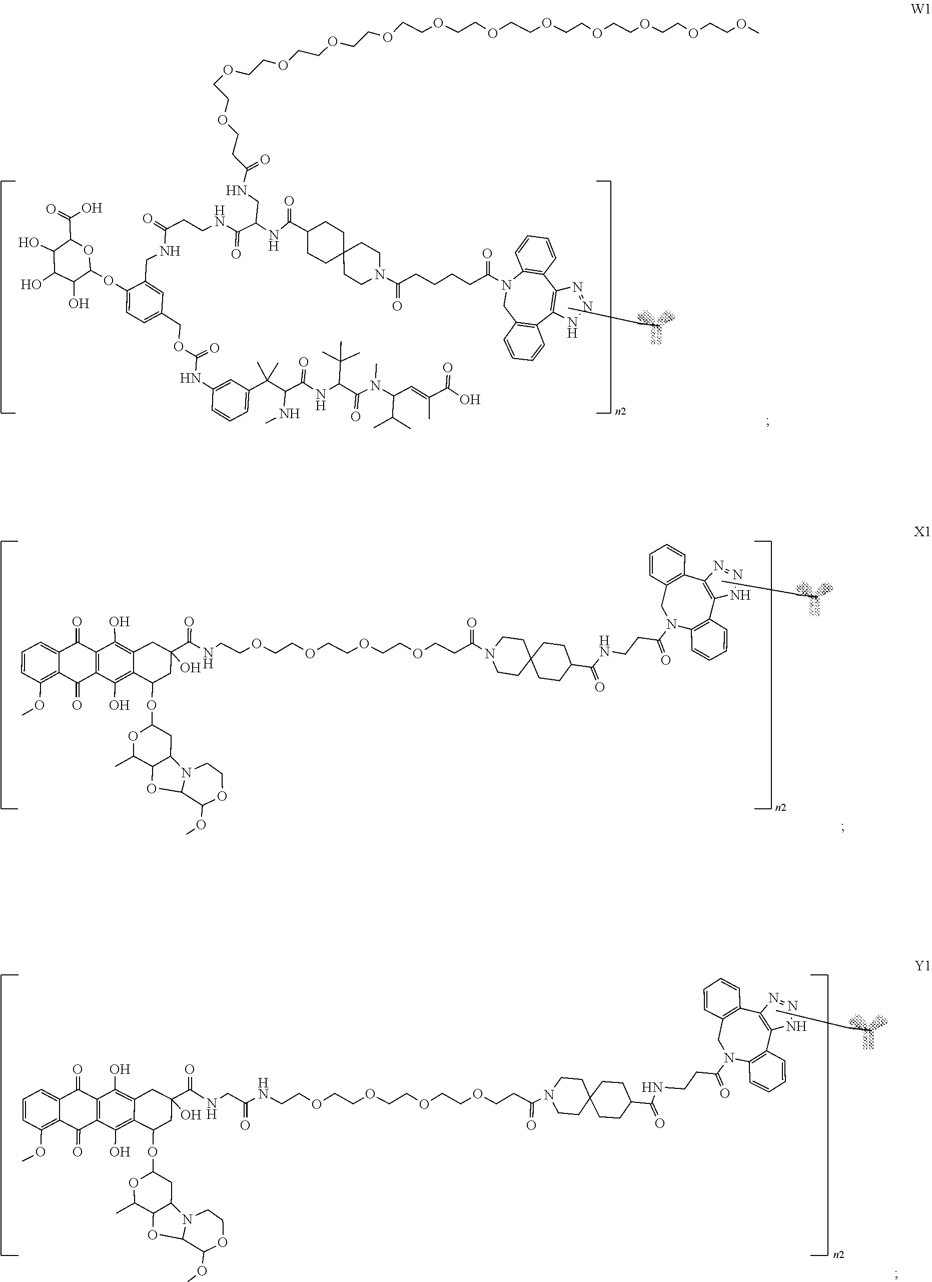 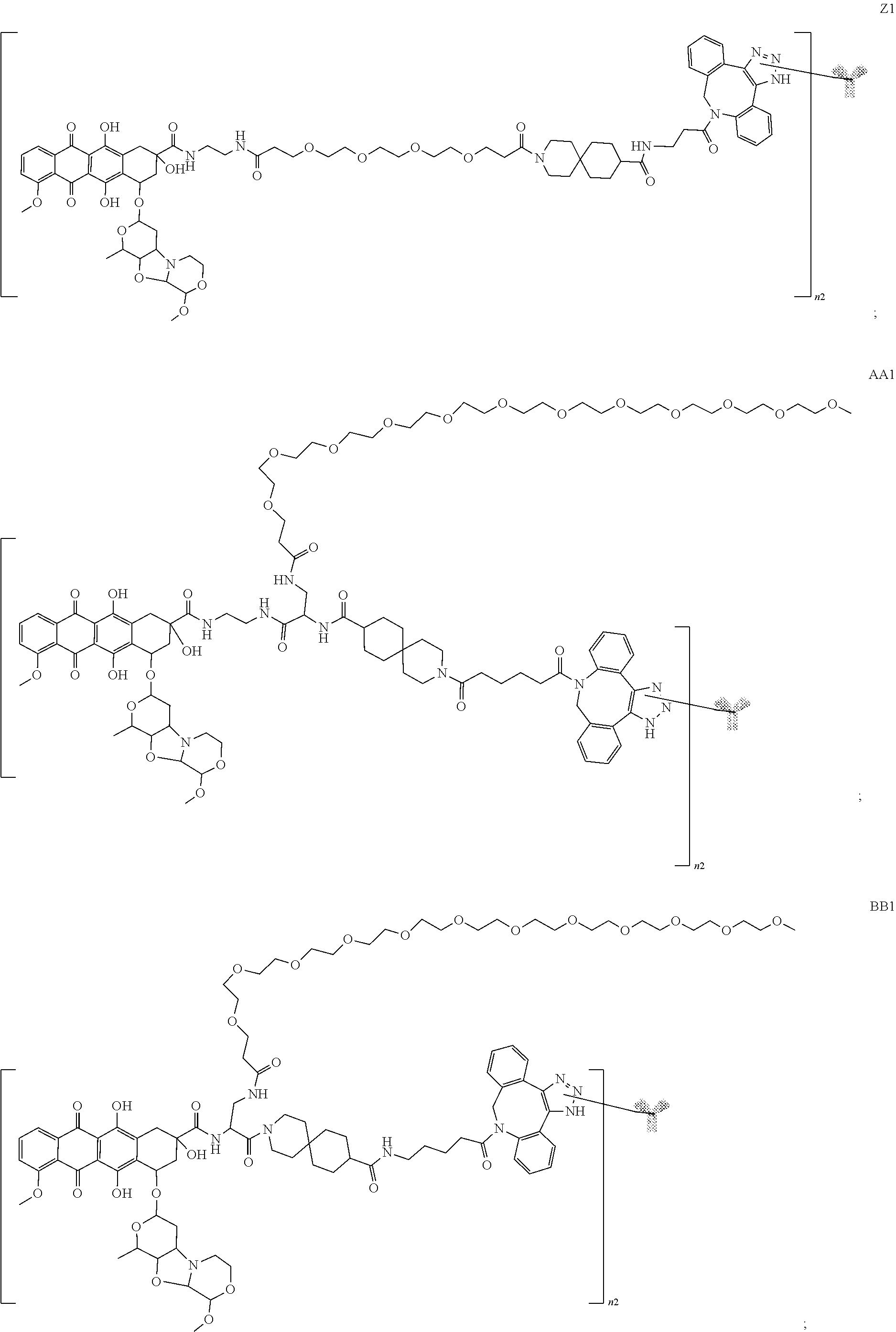 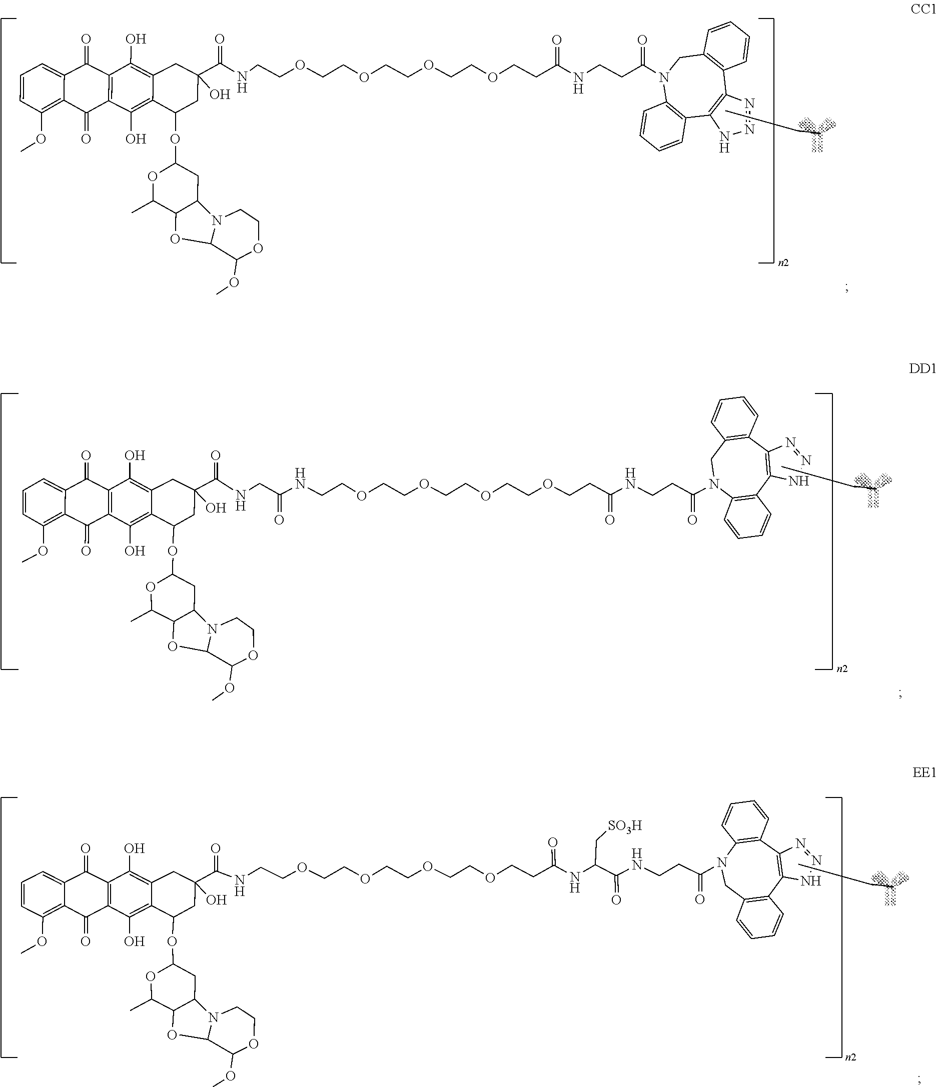 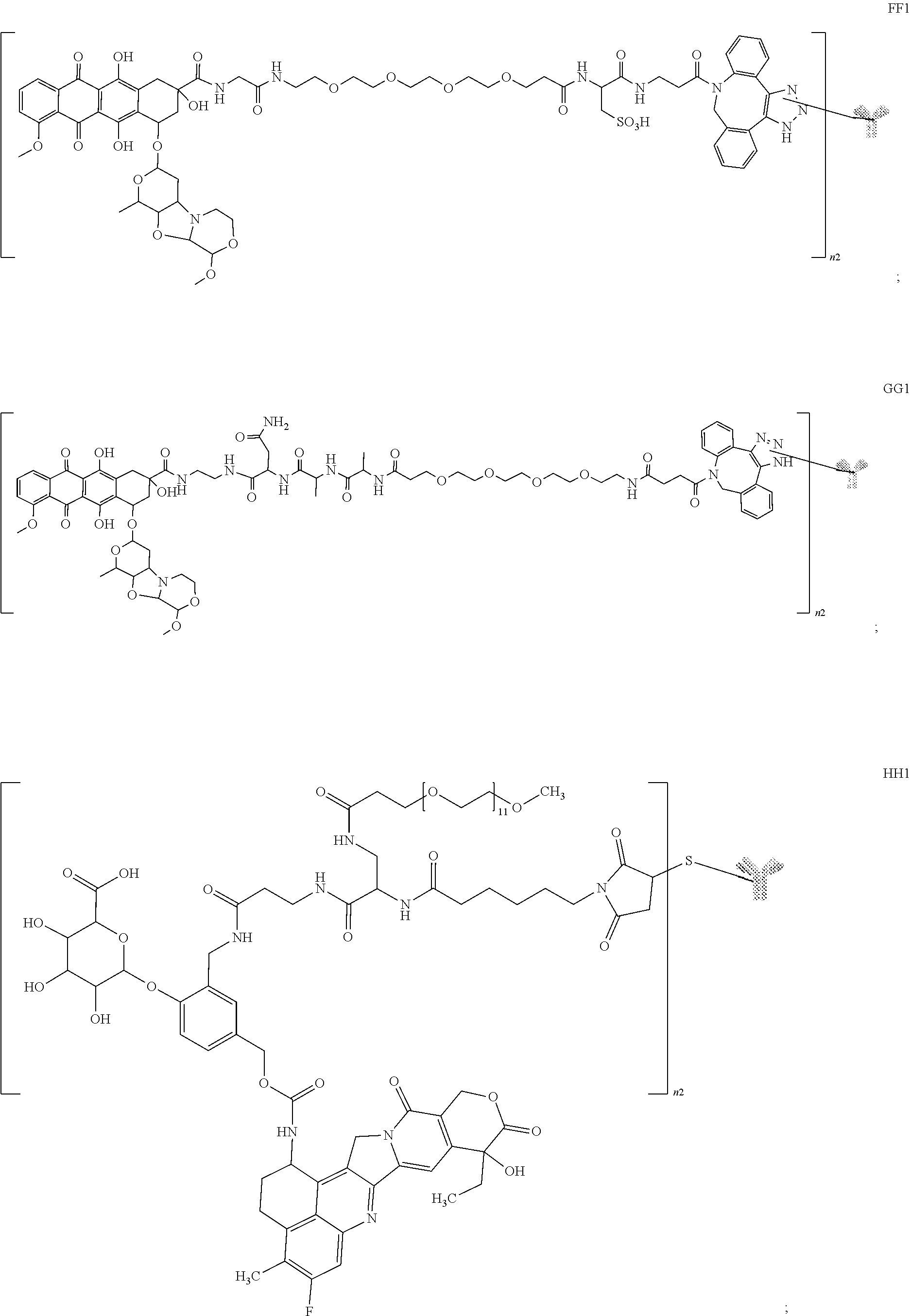 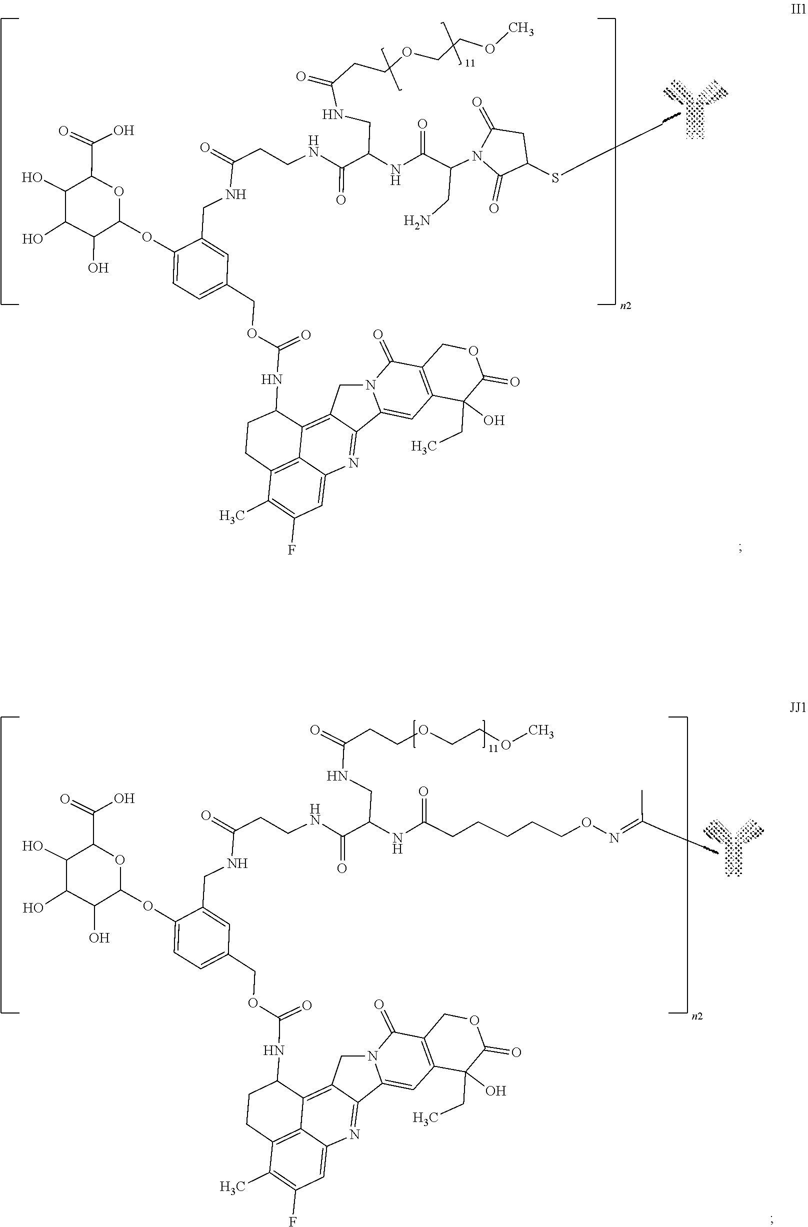 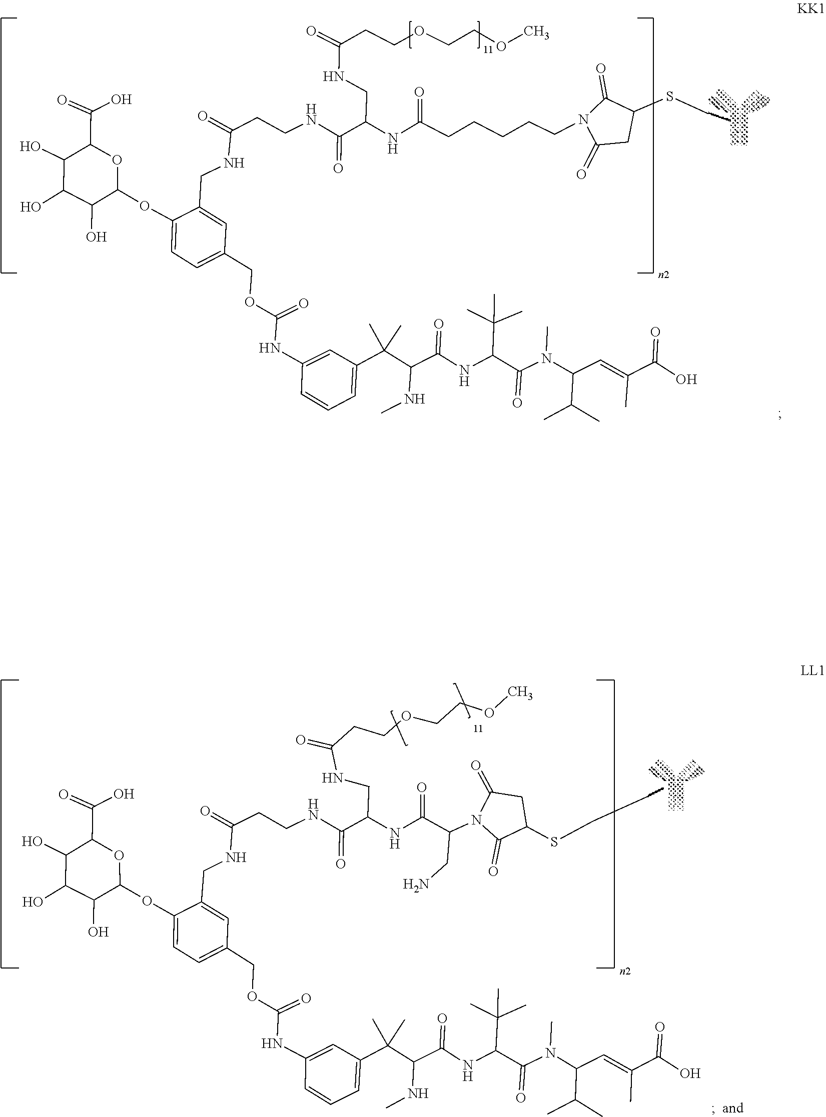 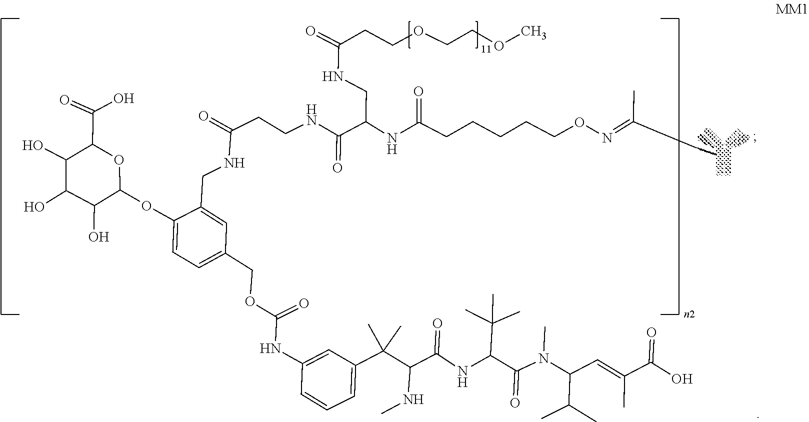 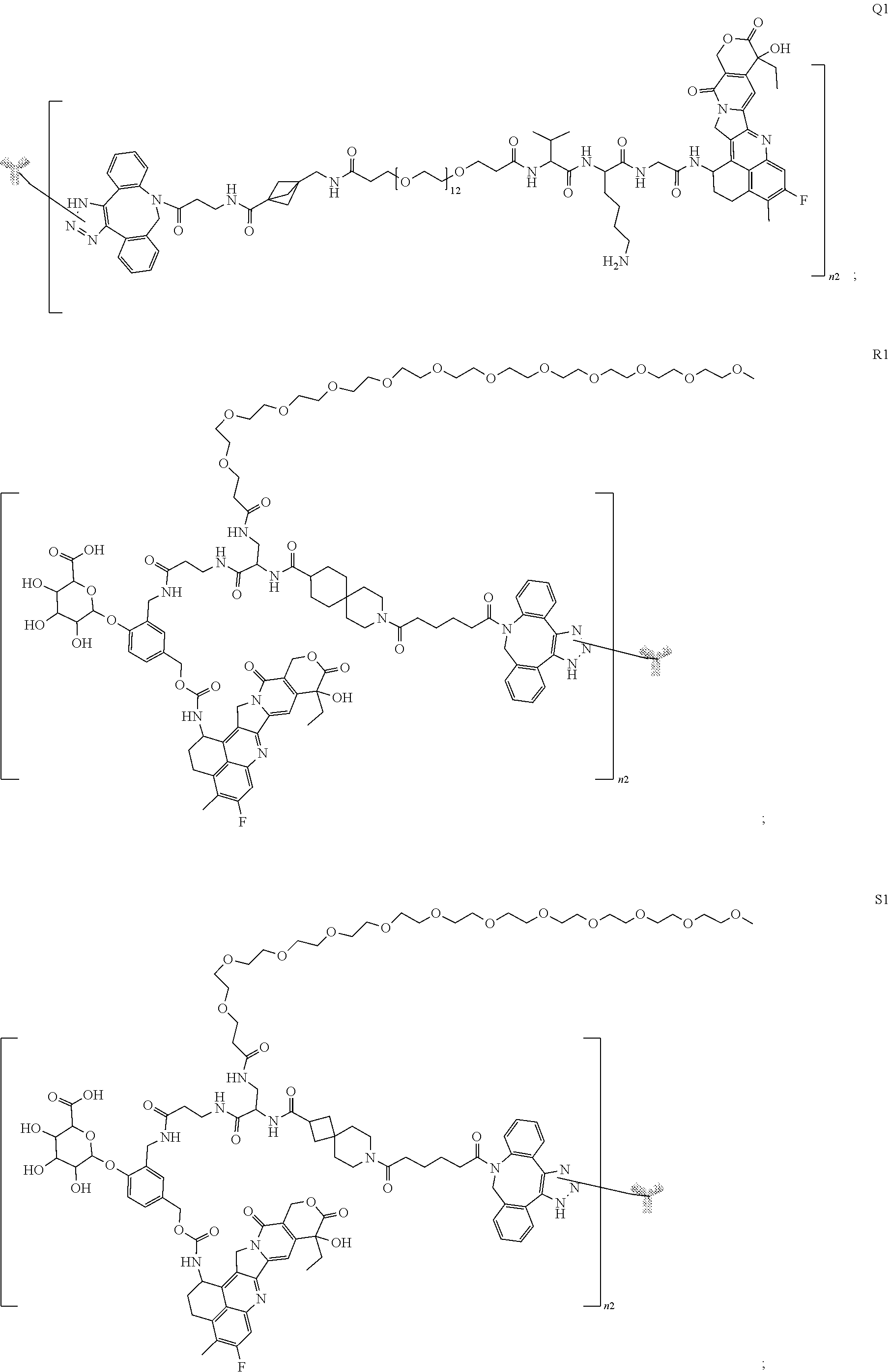 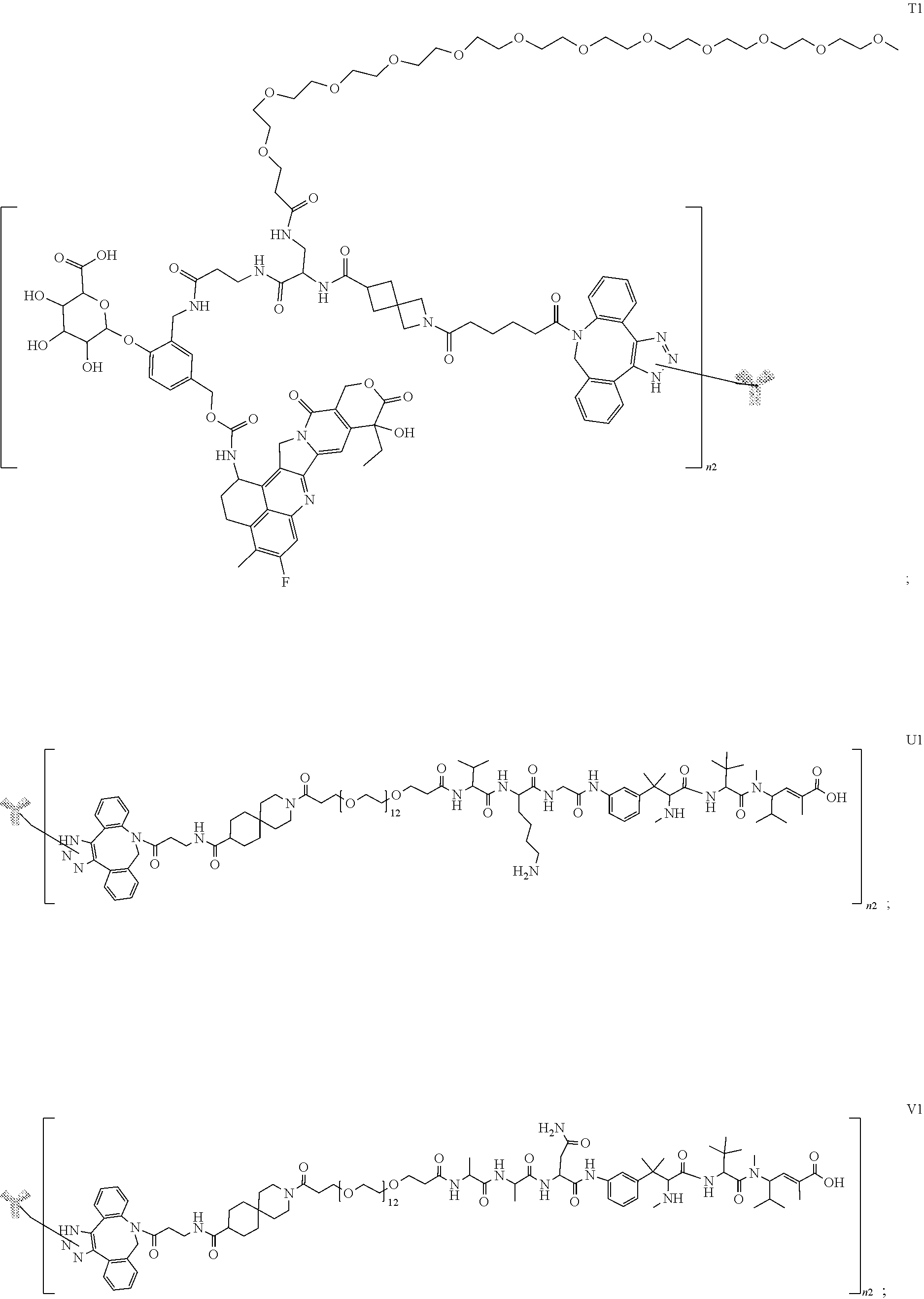 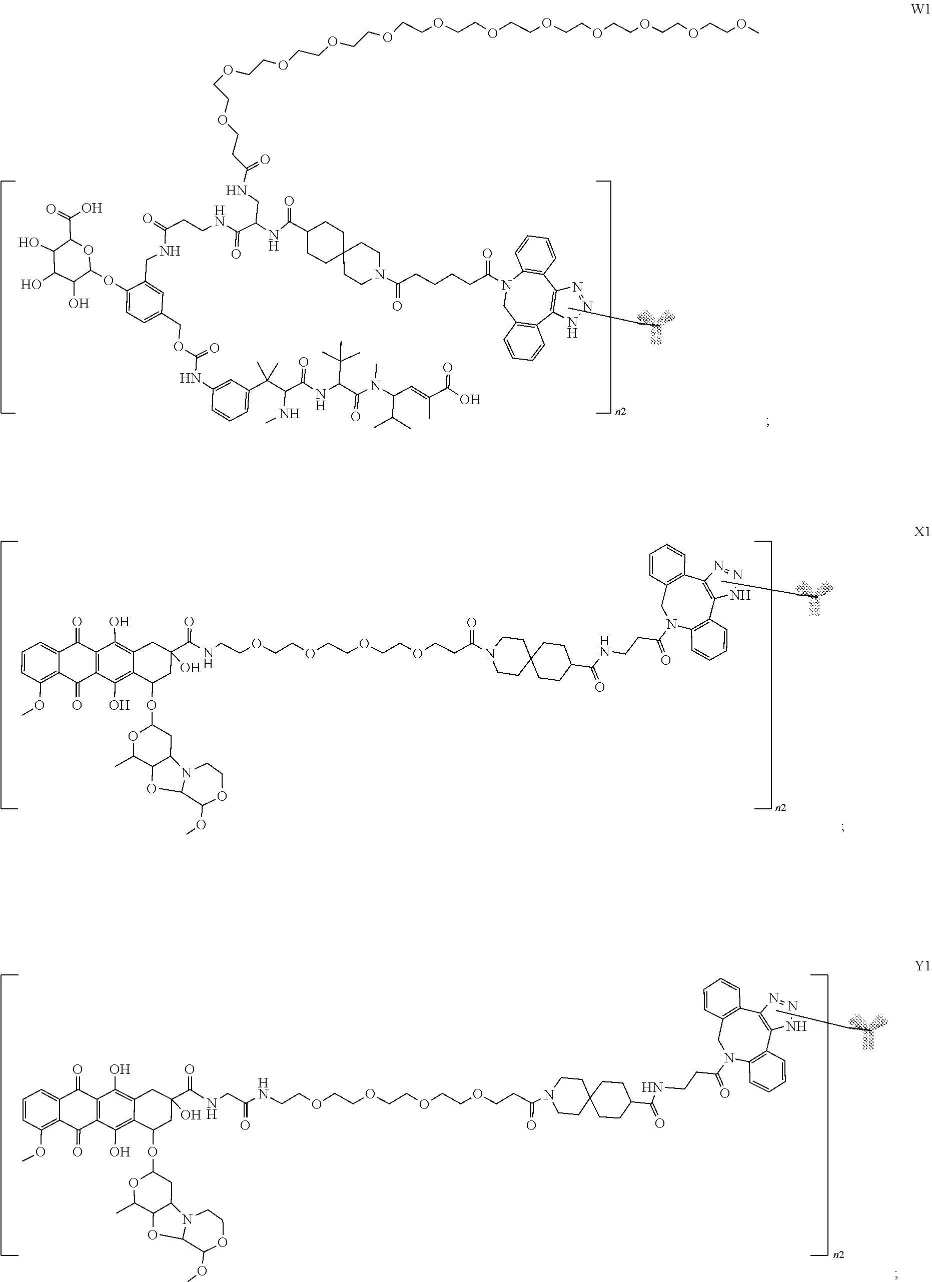 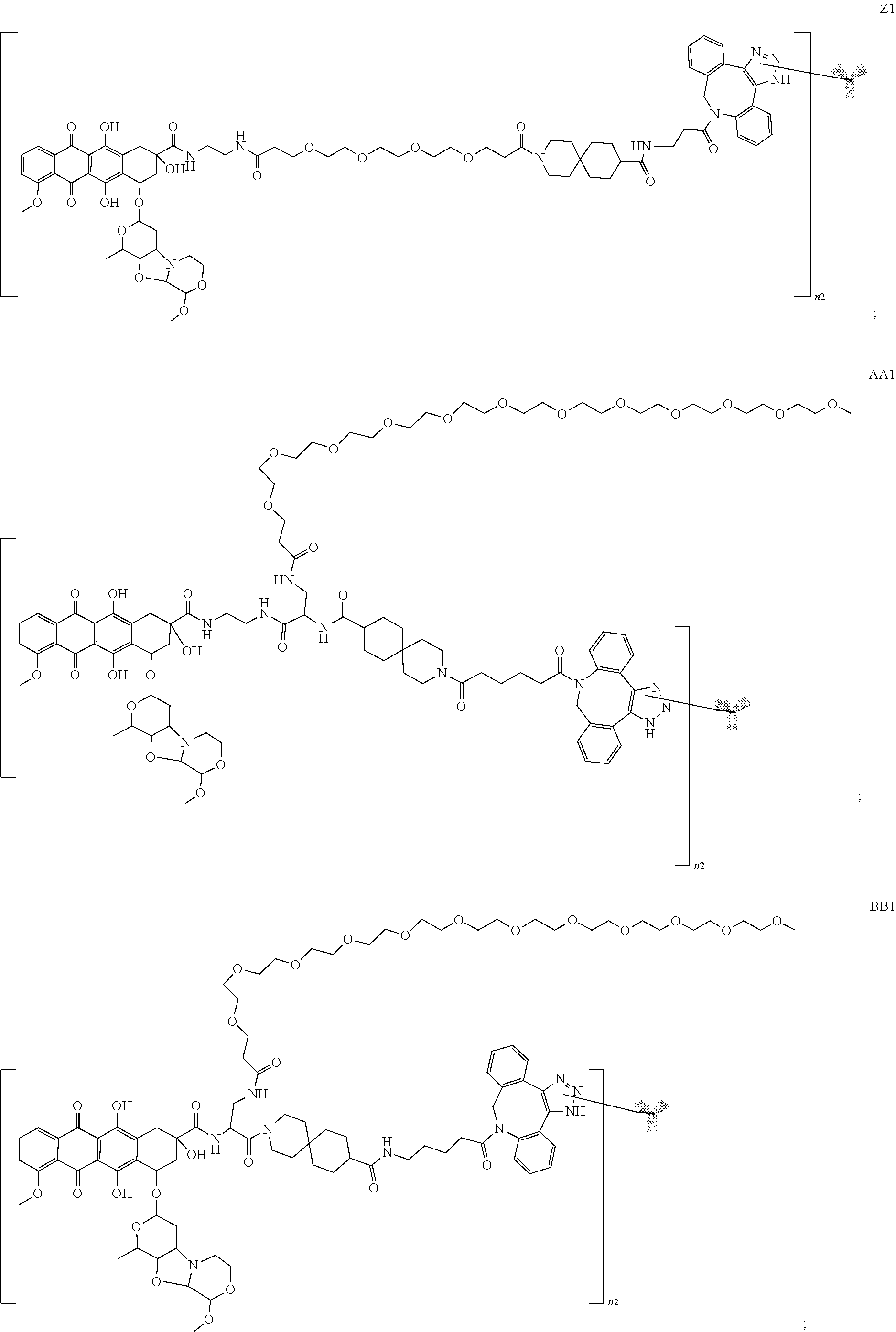 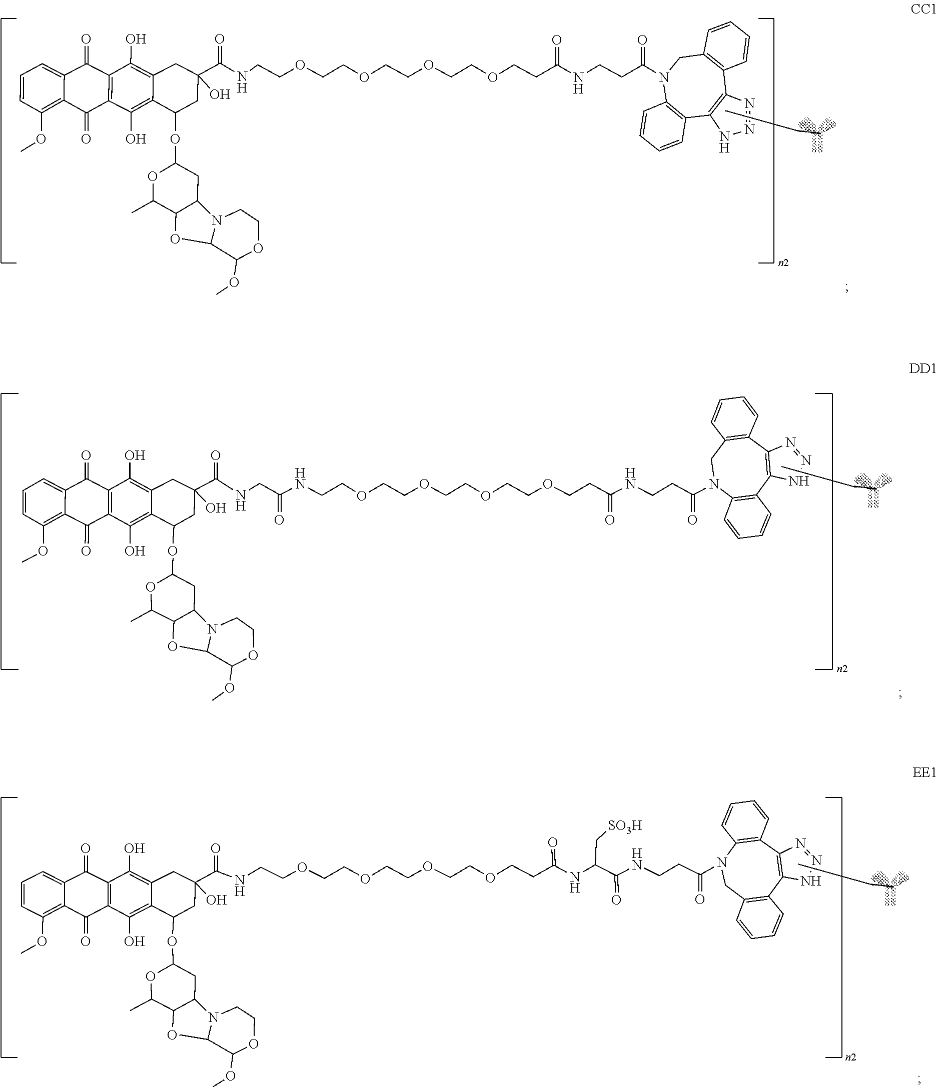
wherein n2 is an integer from 1 to 10.

47. The antibody conjugate of claim 1, wherein the antibody conjugate has a structure selected from the group consisting of: 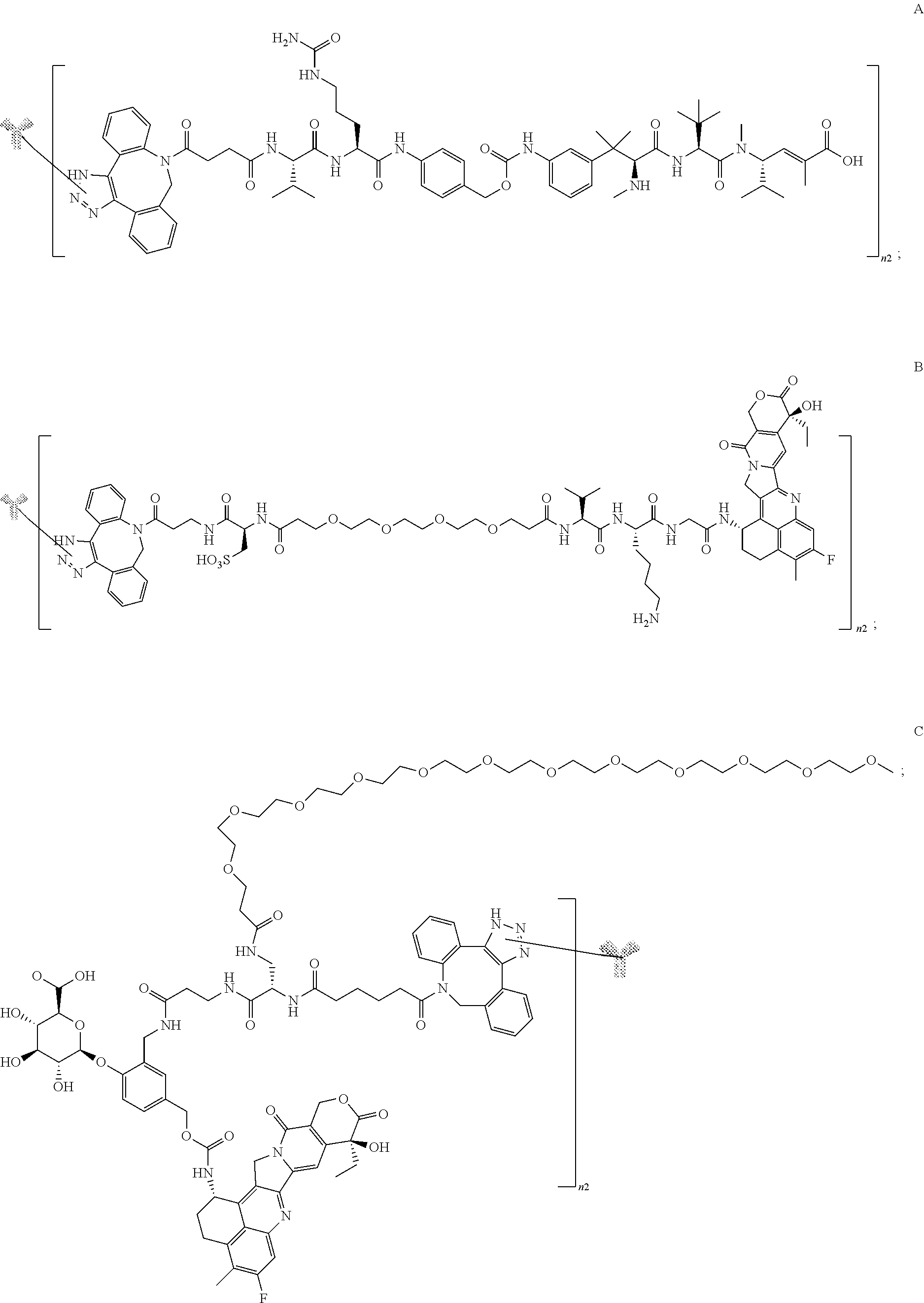 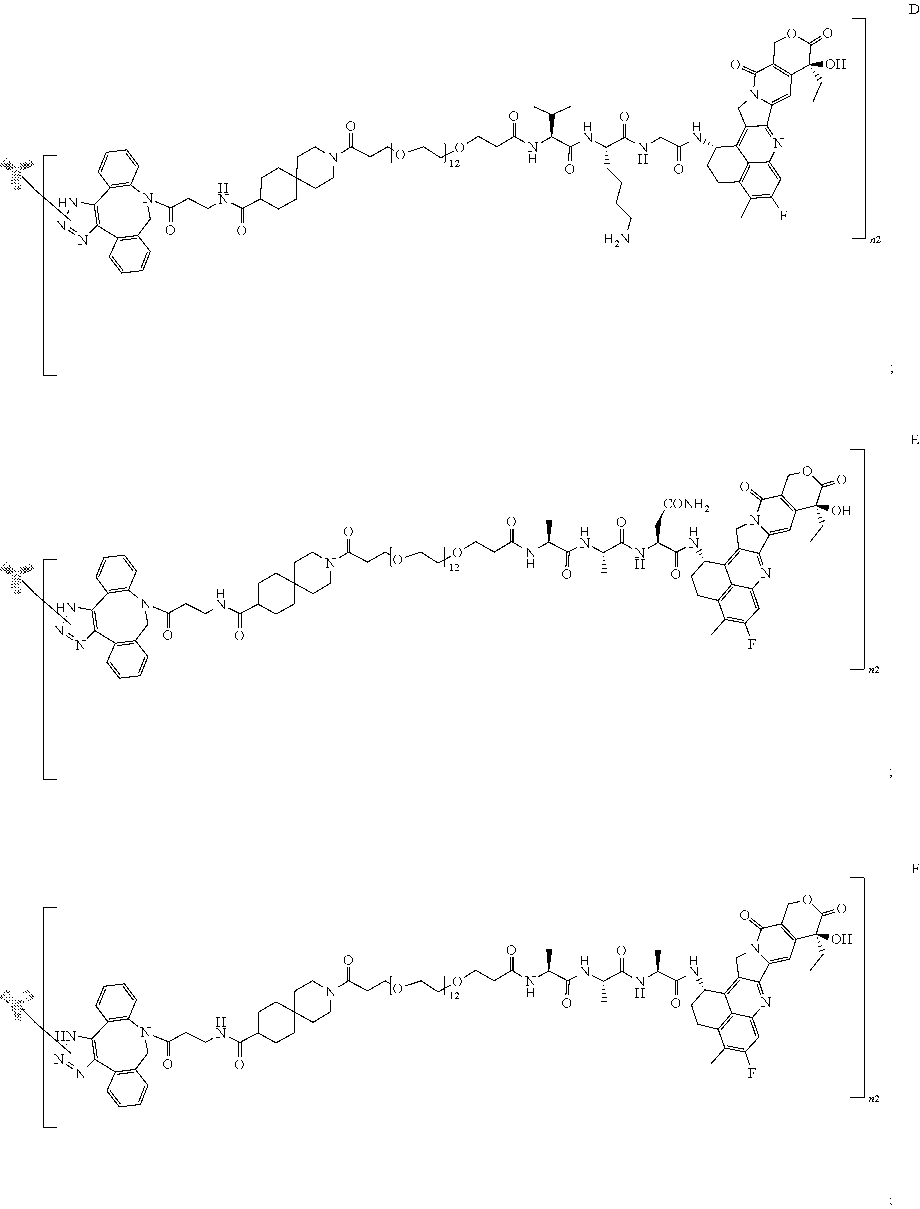 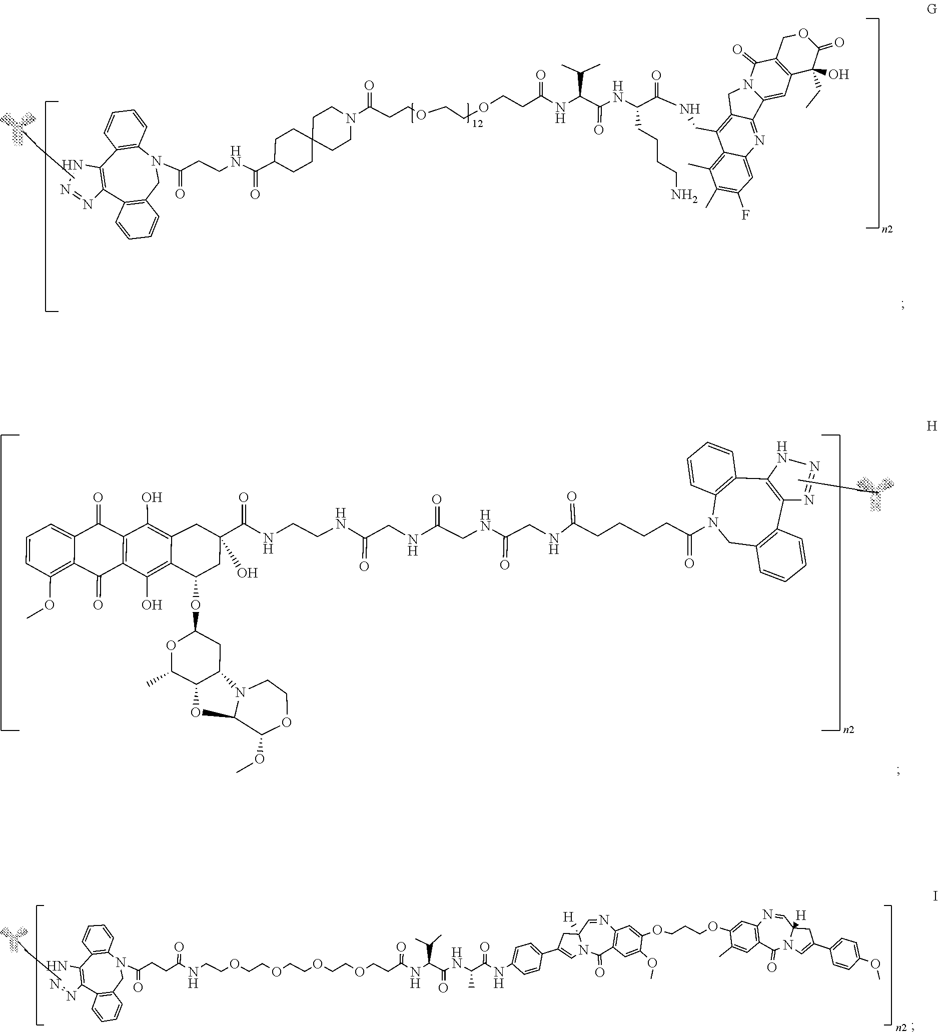 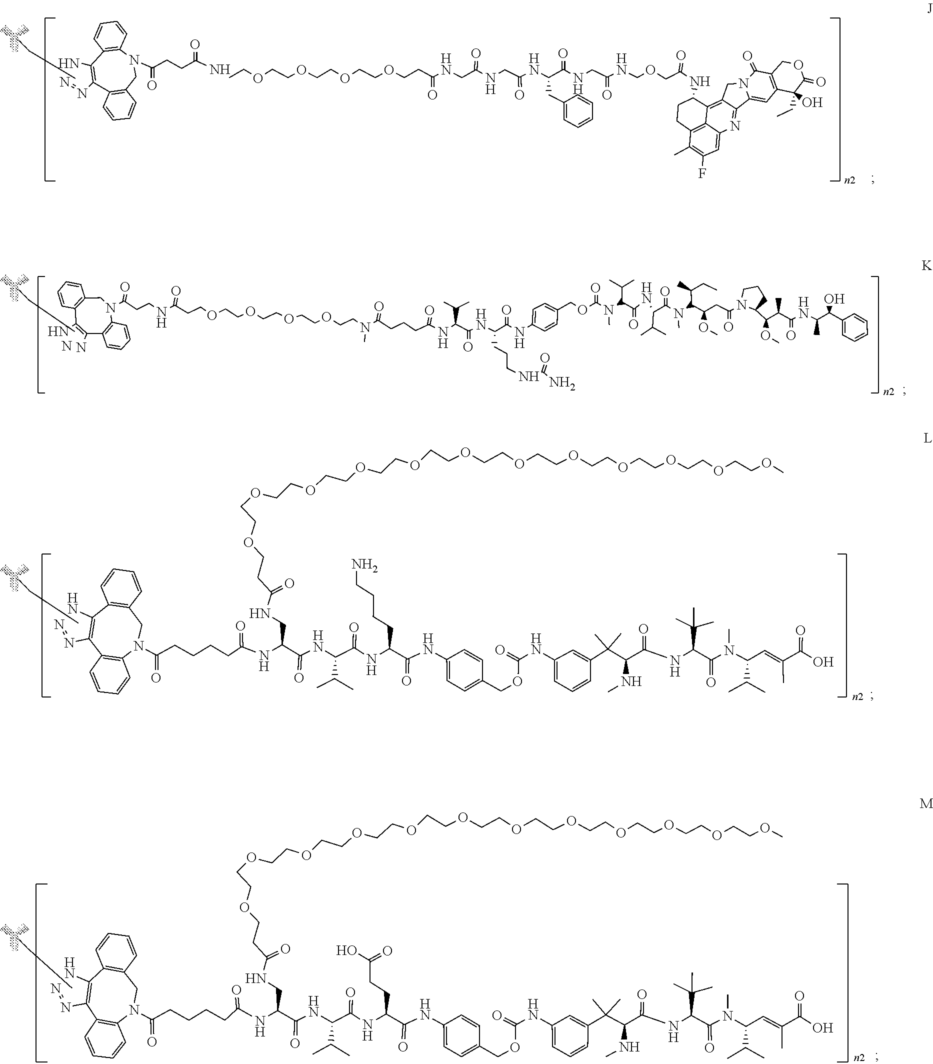 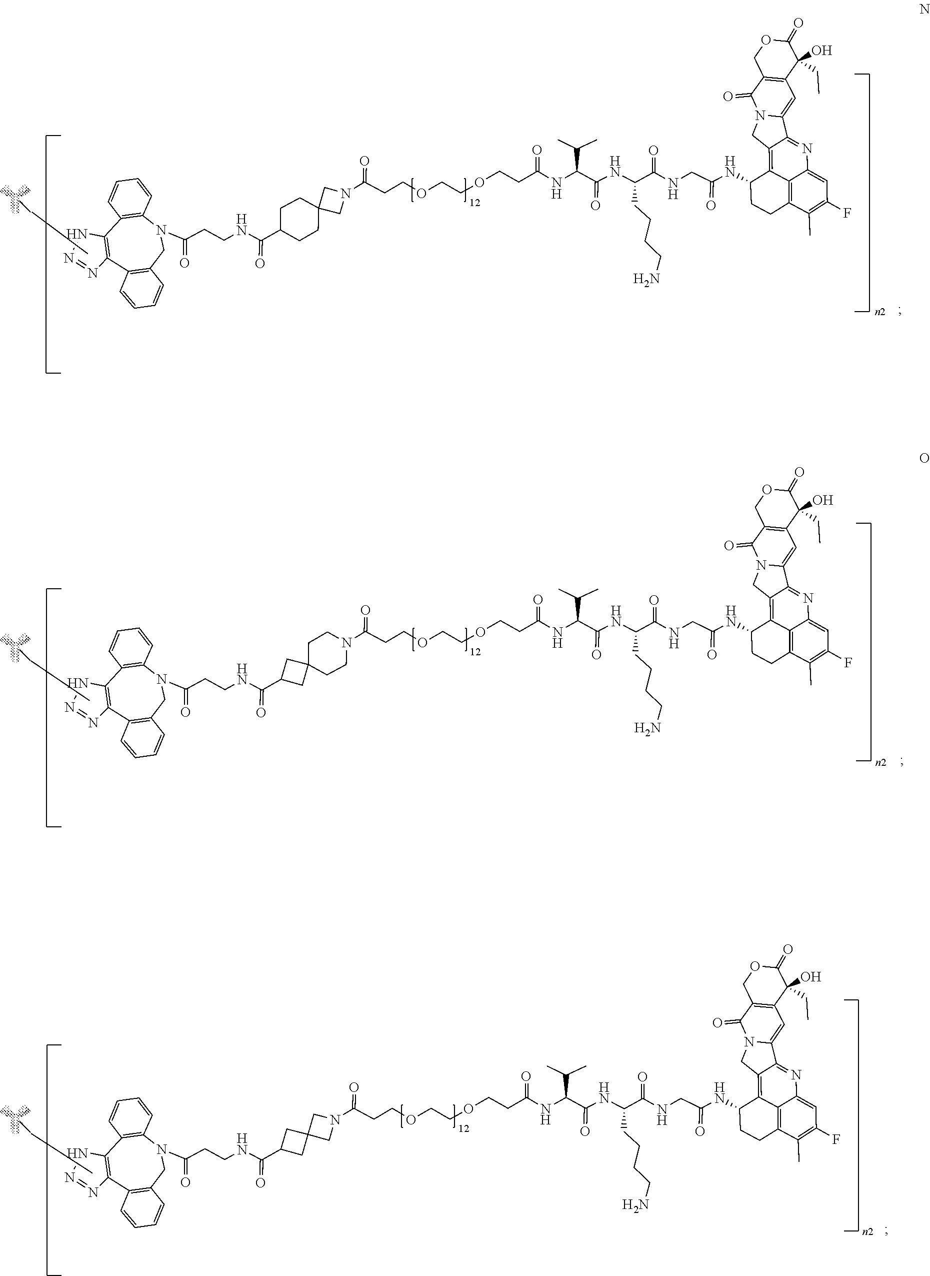 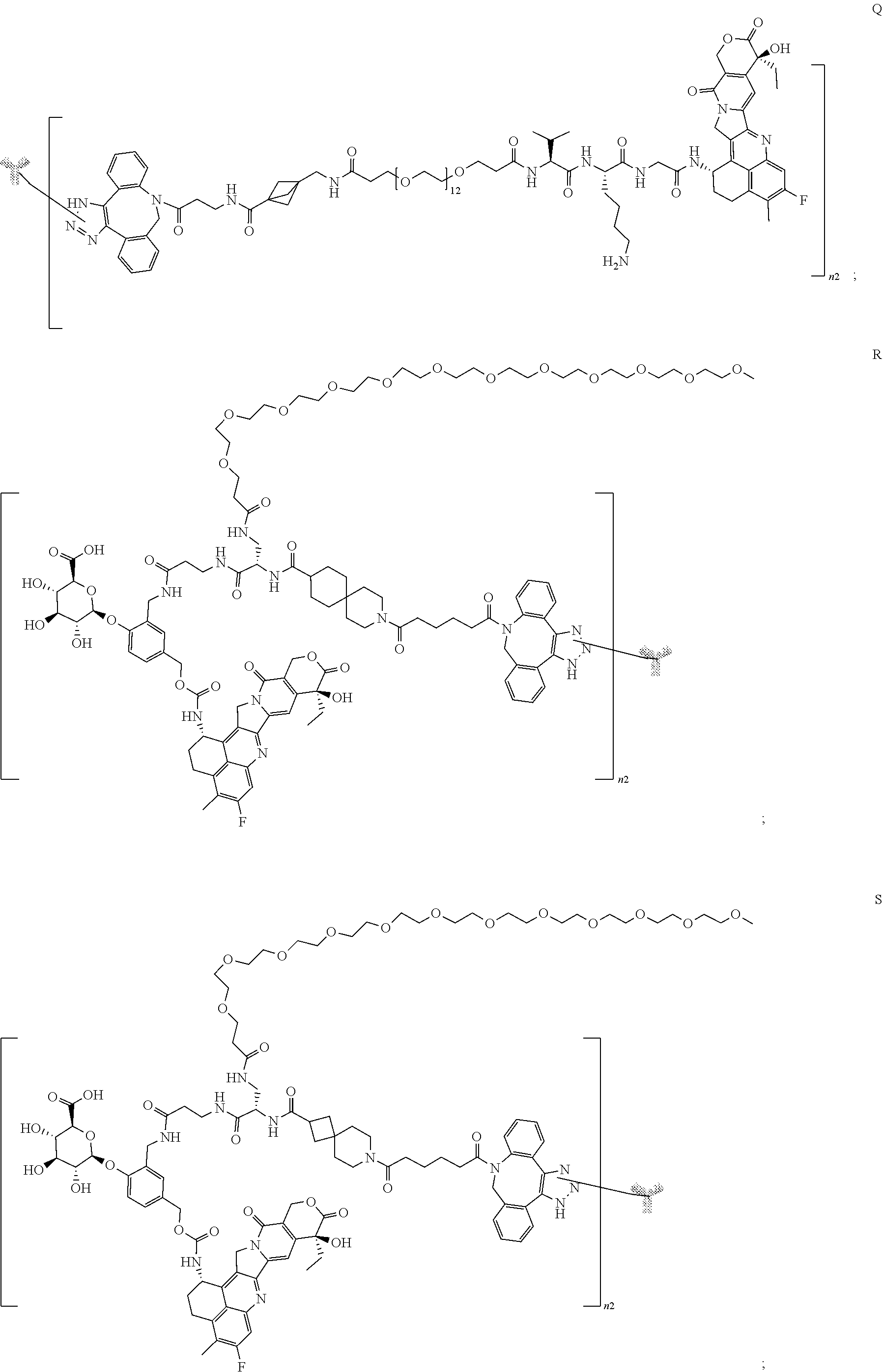 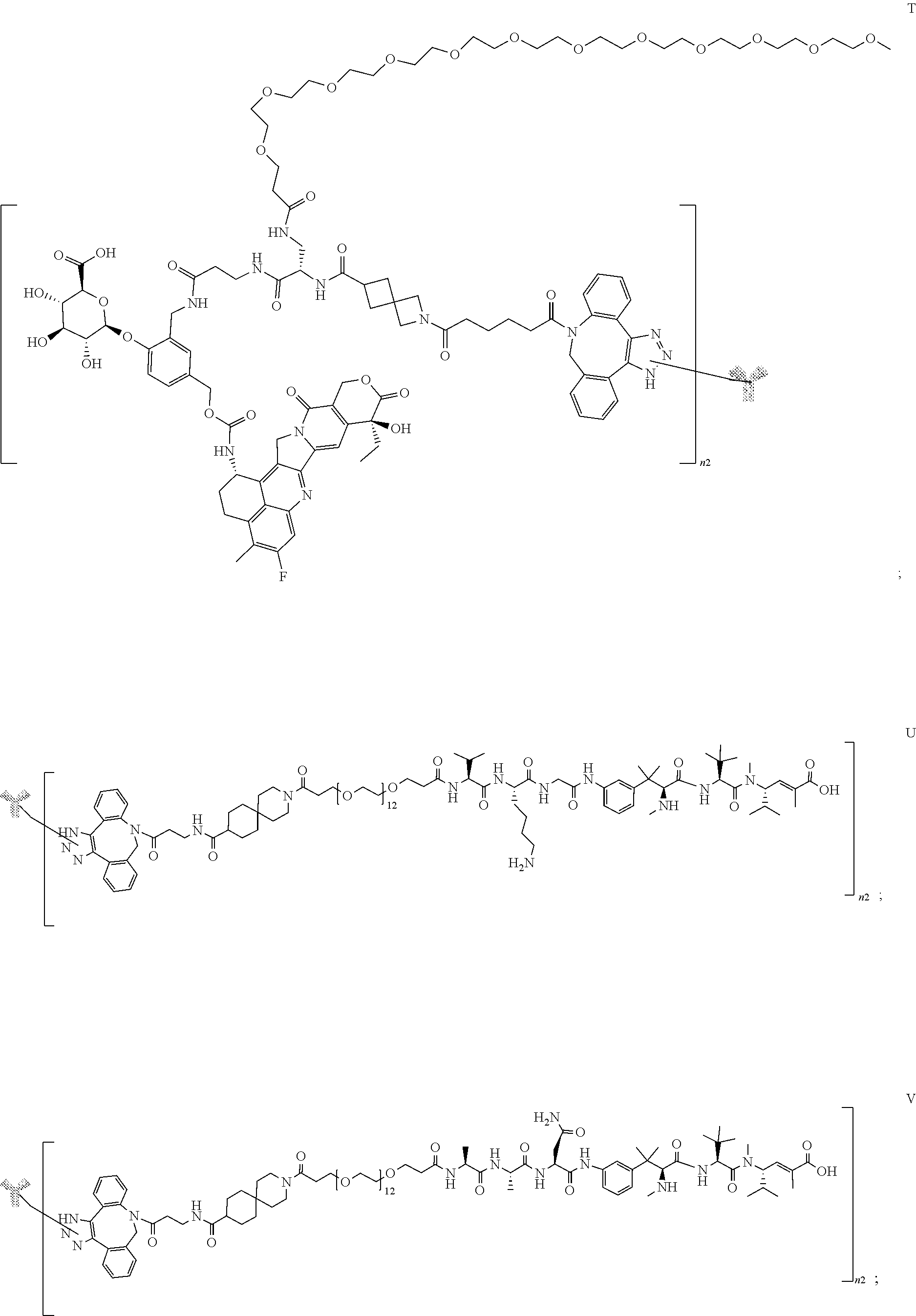 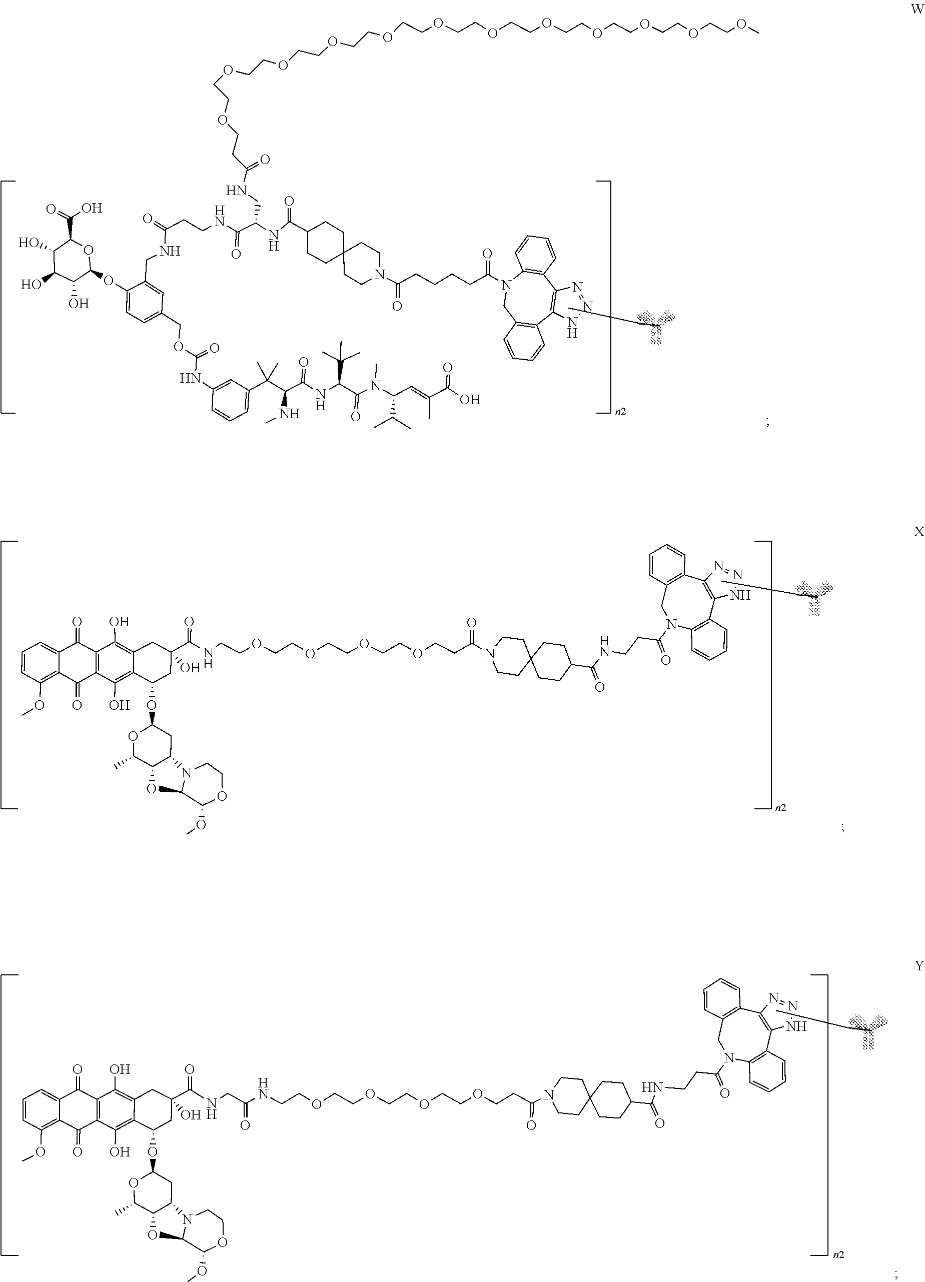 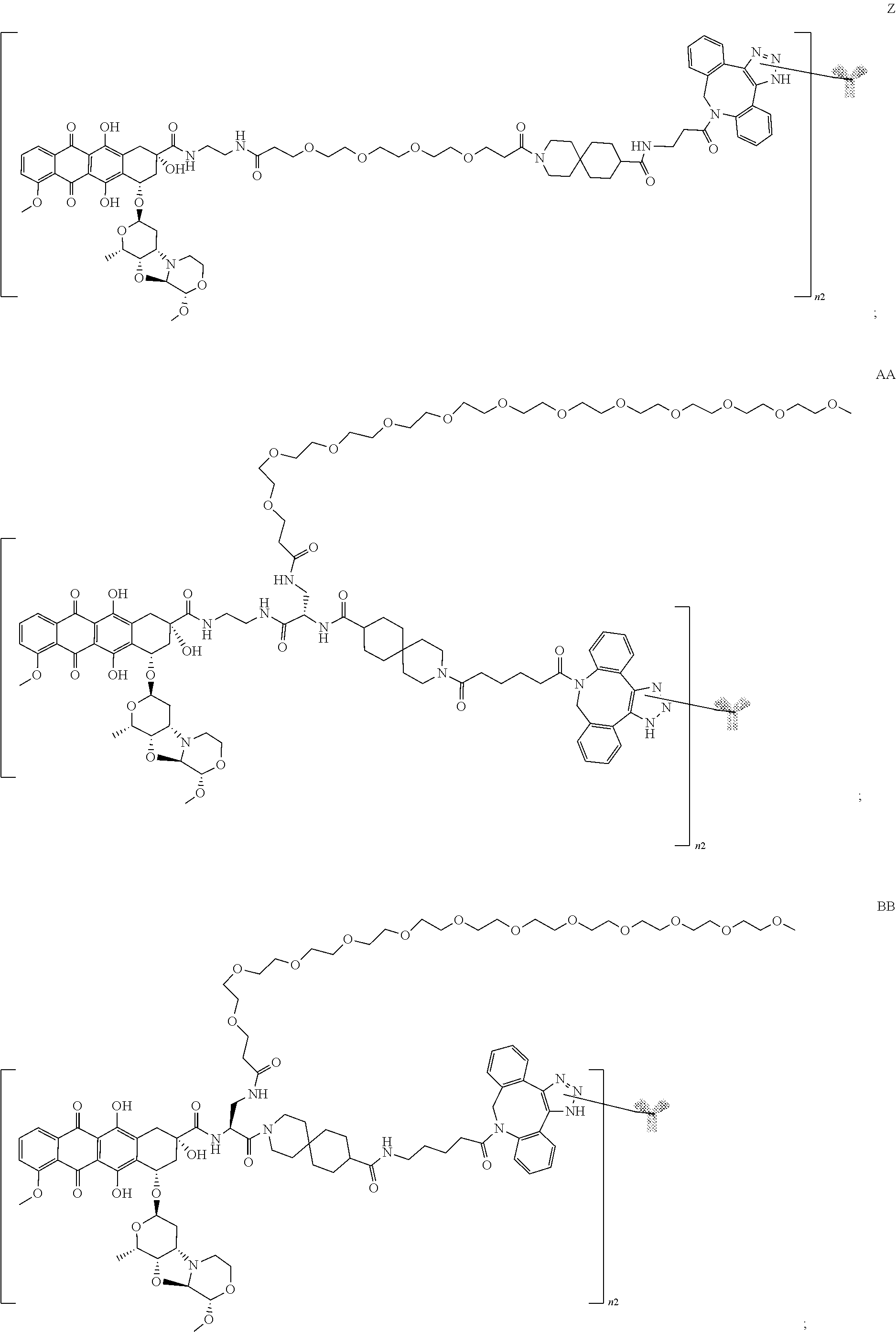 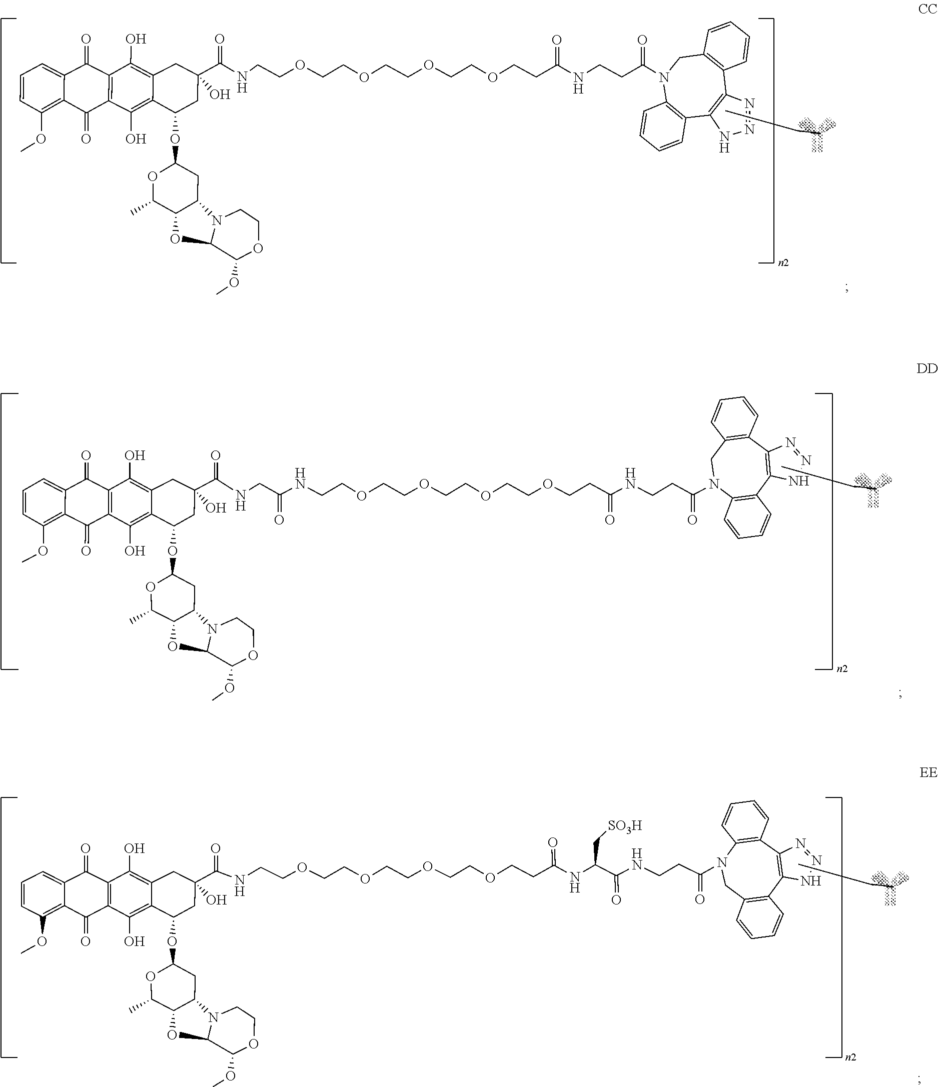 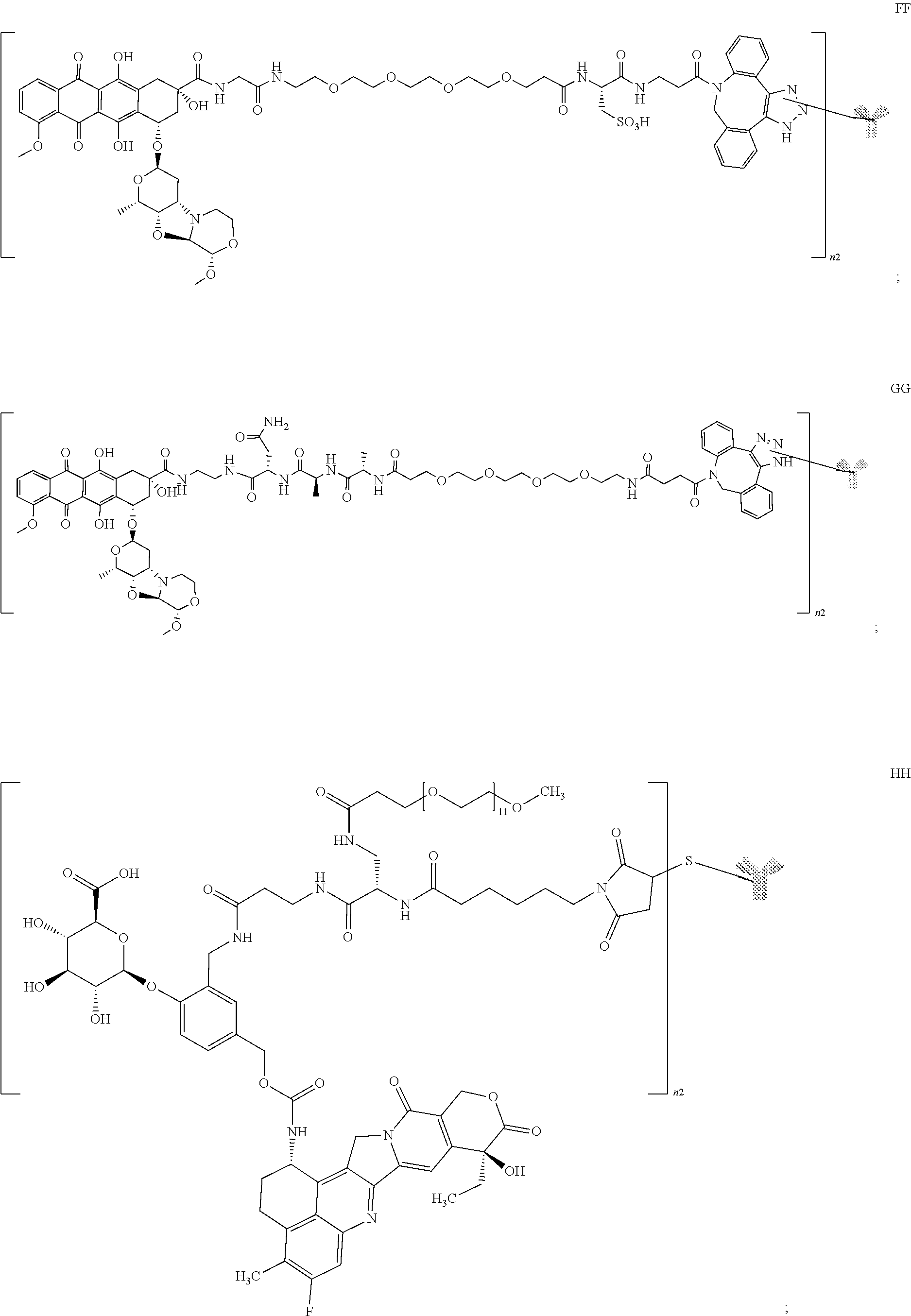 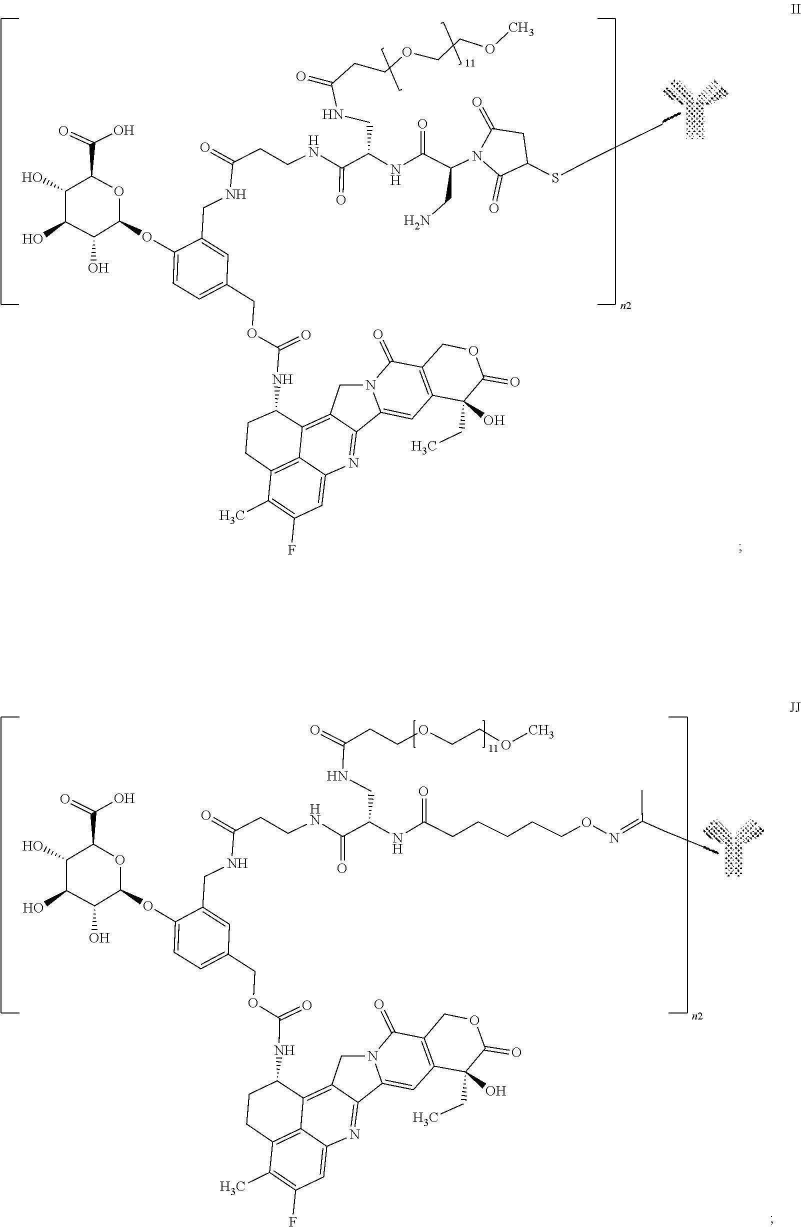 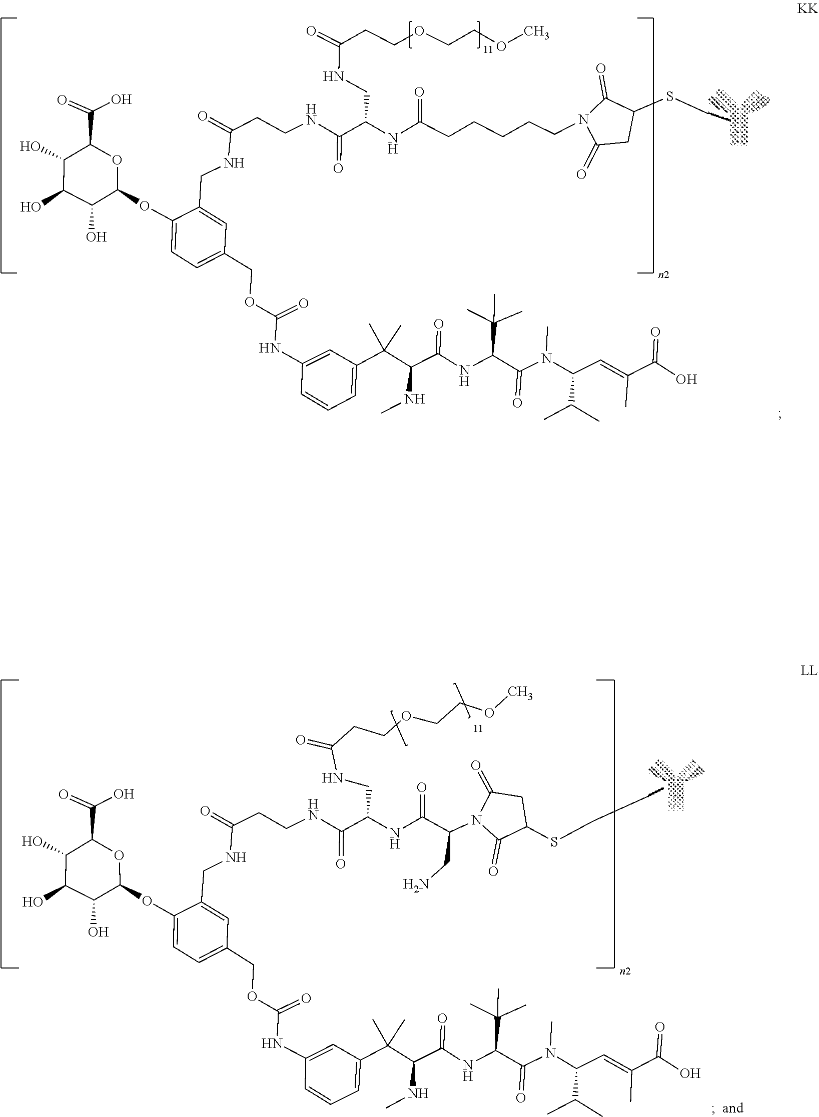 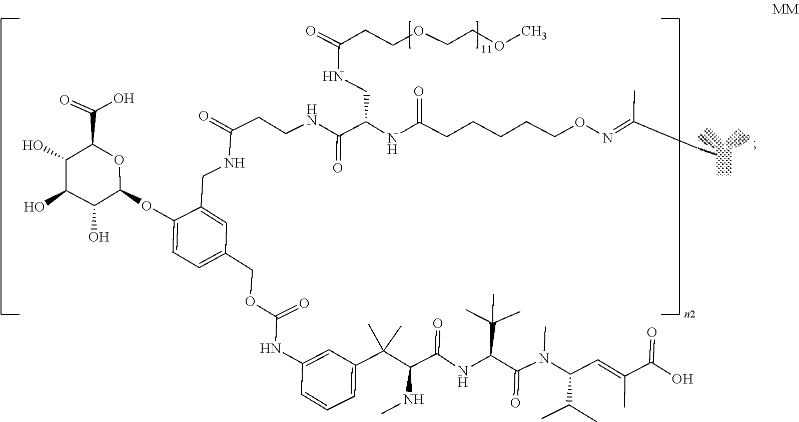
wherein n2 is an integer from 1 to 10.

48. The antibody conjugate of any one of claim 46 or 47, wherein n2 is an integer from 2 to 8, 2 to 6, 2 to 4, or wherein n2 is 2, 4, 6, 8, or 10.

49. The antibody conjugate of any one of the preceding claims, wherein the antibody comprises three heavy chain CDRs from a V_{H} sequence selected from SEQ ID NOs: 854-1020, or variants thereof, and three light chain CDRs from a V_{L} sequence selected from SEQ ID NOs: 1021-1026, or variants thereof.

50. The antibody conjugate of any one of the preceding claims, wherein the antibody comprises three heavy chain CDRs, or variants thereof, and three light chain CDRs, or variants thereof, from a V_{H}/V_{L} pair selected from the group consisting of SEQ ID NOS: 854/1021, 855/1021, 856/1021, 857/1021, 858/1021, 859/1021, 860/1021, 861/1021, 862/1021, 863/1021, 864/1021, 865/1021, 866/1021, 867/1021, 868/1021, 869/1021, 870/1021, 871/1021, 872/1021, 873/1021, 874/1021, 875/1021, 876/1021, 877/1021, 878/1021, 879/1021, 880/1021, 881/1021, 882/1021, 883/1021, 884/1021, 885/1021, 886/1021, 887/1021, 888/1021, 889/1021, 890/1021, 891/1021, 892/1021, 893/1021, 894/1021, 895/1021, 896/1021, 897/1021, 898/1021, 899/1021, 900/1021, 901/1021, 902/1021, 903/1021, 904/1021, 905/1021, 906/1021, 907/1021, 908/1021, 909/1021, 910/1021, 911/1021, 912/1021, 913/1021, 914/1021, 915/1021, 916/1021, 917/1021, 918/1021, 919/1021, 920/1021, 921/1021, 922/1021, 923/1021, 924/1021, 925/1021, 926/1021, 927/1021, 928/1021, 929/1021, 930/1021, 931/1021, 932/1021, 933/1021, 934/1021, 935/1021, 936/1021, 937/1021, 938/1021, 939/1021, 940/1021, 941/1021, 942/1021, 943/1021, 944/1021, 945/1021, 946/1021, 947/1021, 948/1021, 949/1021, 950/1021, 951/1021, 952/1021, 953/1021, 954/1021, 955/1021, 956/1021, 957/1021, 958/1021, 959/1021, 960/1021, 961/1021, 962/1021, 963/1021, 964/1021, 965/1021, 966/1021, 967/1021, 968/1021, 969/1021, 970/1021, 971/1021, 972/1021, 973/1021, 974/1021, 975/1021, 976/1021, 977/1021, 978/1021, 979/1021, 980/1021, 981/1021, 982/1021, 983/1021, 984/1021, 985/1021, 986/1021, 987/1021, 988/1021, 997/1021, 998/1021, 999/1021, 1000/1021, 1001/1021, 1002/1021, 1003/1021, 1004/1021, 1005/1021, 1006/1021, 1007/1021, 1008/1021, 1009/1021, 1010/1021, 1011/1021, 1012/1021, 1013/1021, 1014/1021, 1015/1021, 1016/1021, 1017/1021, 1018/1021, 1019/1021, and 1020/1021.

51. The antibody conjugate of any one of the preceding claims, wherein the antibody comprises three heavy chain CDRs, or variants thereof, and three light chain CDRs, or variants thereof, from a V_{H}/V_{L} pair selected from the group consisting of SEQ ID NOS: 854/1022, 855/1022, 856/1022, 857/1022, 858/1022, 859/1022, 860/1022, 861/1022, 862/1022, 863/1022, 864/1022, 865/1022, 866/1022, 867/1022, 868/1022, 869/1022, 870/1022, 871/1022, 872/1022, 873/1022, 874/1022, 875/1022, 876/1022, 877/1022, 878/1022, 879/1022, 880/1022, 881/1022, 882/1022, 883/1022, 884/1022, 885/1022, 886/1022, 887/1022, 888/1022, 889/1022, 890/1022, 891/1022, 892/1022, 893/1022, 894/1022, 895/1022, 896/1022, 897/1022, 898/1022, 899/1022, 900/1022, 901/1022, 902/1022, 903/1022, 904/1022, 905/1022, 906/1022, 907/1022, 908/1022, 909/1022, 910/1022, 911/1022, 912/1022, 913/1022, 914/1022, 915/1022, 916/1022, 917/1022, 918/1022, 919/1022, 920/1022, 921/1022, 922/1022, 923/1022, 924/1022, 925/1022, 926/1022, 927/1022, 928/1022, 929/1022, 930/1022, 931/1022, 932/1022, 933/1022, 934/1022, 935/1022, 936/1022, 937/1022, 938/1022, 939/1022, 940/1022, 941/1022, 942/1022, 943/1022, 944/1022, 945/1022, 946/1022, 947/1022, 948/1022, 949/1022, 950/1022, 951/1022, 952/1022, 953/1022, 954/1022, 955/1022, 956/1022, 957/1022, 958/1022, 959/1022, 960/1022, 961/1022, 962/1022, 963/1022, 964/1022, 965/1022, 966/1022, 967/1022, 968/1022, 969/1022, 970/1022, 971/1022, 972/1022, 973/1022, 974/1022, 975/1022, 976/1022, 977/1022, 978/1022, 979/1022, 980/1022, 981/1022, 982/1022, 983/1022, 984/1022, 985/1022, 986/1022, 987/1022, 988/1022, 989/1022, 990/1022, 991/1022, 992/1022, 993/1022, 994/1022, 995/1022, 996/1022, 997/1022, 998/1022, 999/1022, 1000/1022, 1001/1022, 1002/1022, 1003/1022, 1004/1022, 1005/1022, 1006/1022, 1007/1022, 1008/1022, 1009/1022, 1010/1022, 1011/1022, 1012/1022, 1013/1022, 1014/1022, 1015/1022, 1016/1022, 1017/1022, 1018/1022, 1019/1022, and 1020/1022.

52. The antibody conjugate of any one of the preceding claims, wherein the antibody comprises three heavy chain CDRs, or variants thereof, and three light chain CDRs, or variants thereof, from a V_{H}/V_{L} pair selected from the group consisting of SEQ ID NOS: 854/1023, 855/1023, 856/1023, 857/1023, 858/1023, 859/1023, 860/1023, 861/1023, 862/1023, 863/1023, 864/1023, 865/1023, 866/1023, 867/1023, 868/1023, 869/1023, 870/1023, 871/1023, 872/1023, 873/1023, 874/1023, 875/1023, 876/1023, 877/1023, 878/1023, 879/1023, 880/1023, 881/1023, 882/1023, 883/1023, 884/1023, 885/1023, 886/1023, 887/1023, 888/1023, 889/1023, 890/1023, 891/1023, 892/1023, 893/1023, 894/1023, 895/1023, 896/1023, 897/1023, 898/1023, 899/1023, 900/1023, 901/1023, 902/1023, 903/1023, 904/1023, 905/1023, 906/1023, 907/1023, 908/1023, 909/1023, 910/1023, 911/1023, 912/1023, 913/1023, 914/1023, 915/1023, 916/1023, 917/1023, 918/1023, 919/1023, 920/1023, 921/1023, 922/1023, 923/1023, 924/1023, 925/1023, 926/1023, 927/1023, 928/1023, 929/1023, 930/1023, 931/1023, 932/1023, 933/1023, 934/1023, 935/1023, 936/1023, 937/1023, 938/1023, 939/1023, 940/1023, 941/1023, 942/1023, 943/1023, 944/1023, 945/1023, 946/1023, 947/1023, 948/1023, 949/1023, 950/1023, 951/1023, 952/1023, 953/1023, 954/1023, 955/1023, 956/1023, 957/1023, 958/1023, 959/1023, 960/1023, 961/1023, 962/1023, 963/1023, 964/1023, 965/1023, 966/1023, 967/1023, 968/1023, 969/1023, 970/1023, 971/1023, 972/1023, 973/1023, 974/1023, 975/1023, 976/1023, 977/1023, 978/1023, 979/1023, 980/1023, 981/1023, 982/1023, 983/1023, 984/1023, 985/1023, 986/1023, 987/1023, 988/1023, 989/1023, 990/1023, 991/1023, 992/1023, 993/1023, 994/1023, 995/1023, 996/1023, 997/1023, 998/1023, 999/1023, 1000/1023, 1001/1023, 1002/1023, 1003/1023, 1004/1023, 1005/1023, 1006/1023, 1007/1023, 1008/1023, 1009/1023, 1010/1023, 1011/1023, 1012/1023, 1013/1023, 1014/1023, 1015/1023, 1016/1023, 1017/1023, 1018/1023, 1019/1023, and 1020/1023.

53. The antibody conjugate of any one of the preceding claims, wherein the antibody comprises three heavy chain CDRs, or variants thereof, and three light chain CDRs, or variants thereof, from a V_{H}/V_{L} pair selected from the group consisting of SEQ ID NOS: 854/1024, 855/1024, 856/1024, 857/1024, 858/1024, 859/1024, 860/1024, 861/1024, 862/1024, 863/1024, 864/1024, 865/1024, 866/1024, 867/1024, 868/1024, 869/1024, 870/1024, 871/1024, 872/1024, 873/1024, 874/1024, 875/1024, 876/1024, 877/1024, 878/1024, 879/1024, 880/1024, 881/1024, 882/1024, 883/1024, 884/1024, 885/1024, 886/1024, 887/1024, 888/1024, 889/1024, 890/1024, 891/1024, 892/1024, 893/1024, 894/1024, 895/1024, 896/1024, 897/1024, 898/1024, 899/1024, 900/1024, 901/1024, 902/1024, 903/1024, 904/1024, 905/1024, 906/1024, 907/1024, 908/1024, 909/1024, 910/1024, 911/1024, 912/1024, 913/1024, 914/1024, 915/1024, 916/1024, 917/1024, 918/1024, 919/1024, 920/1024, 921/1024, 922/1024, 923/1024, 924/1024, 925/1024, 926/1024, 927/1024, 928/1024, 929/1024, 930/1024, 931/1024, 932/1024, 933/1024, 934/1024, 935/1024, 936/1024, 937/1024, 938/1024, 939/1024, 940/1024, 941/1024, 942/1024, 943/1024, 944/1024, 945/1024, 946/1024, 947/1024, 948/1024, 949/1024, 950/1024, 951/1024, 952/1024, 953/1024, 954/1024, 955/1024, 956/1024, 957/1024, 958/1024, 959/1024, 960/1024, 961/1024, 962/1024, 963/1024, 964/1024, 965/1024, 966/1024, 967/1024, 968/1024, 969/1024, 970/1024, 971/1024, 972/1024, 973/1024, 974/1024, 975/1024, 976/1024, 977/1024, 978/1024, 979/1024, 980/1024, 981/1024, 982/1024, 983/1024, 984/1024, 985/1024, 986/1024, 987/1024, 988/1024, 989/1024, 990/1024, 991/1024, 992/1024, 993/1024, 994/1024, 995/1024, 996/1024, 997/1024, 998/1024, 999/1024, 1000/1024, 1001/1024, 1002/1024, 1003/1024, 1004/1024, 1005/1024, 1006/1024, 1007/1024, 1008/1024, 1009/1024, 1010/1024, 1011/1024, 1012/1024, 1013/1024, 1014/1024, 1015/1024, 1016/1024, 1017/1024, 1018/1024, 1019/1024, and 1020/1024.

54. The antibody conjugate of any one of the preceding claims, wherein the antibody comprises three heavy chain CDRs, or variants thereof, and three light chain CDRs, or variants thereof, from a V_{H}/V_{L} pair selected from the group consisting of SEQ ID NOS: 854/1025, 855/1025, 856/1025, 857/1025, 858/1025, 859/1025, 860/1025, 861/1025, 862/1025, 863/1025, 864/1025, 865/1025, 866/1025, 867/1025, 868/1025, 869/1025, 870/1025, 871/1025, 872/1025, 873/1025, 874/1025, 875/1025, 876/1025, 877/1025, 878/1025, 879/1025, 880/1025, 881/1025, 882/1025, 883/1025, 884/1025, 885/1025, 886/1025, 887/1025, 888/1025, 889/1025, 890/1025, 891/1025, 892/1025, 893/1025, 894/1025, 895/1025, 896/1025, 897/1025, 898/1025, 899/1025, 900/1025, 901/1025, 902/1025, 903/1025, 904/1025, 905/1025, 906/1025, 907/1025, 908/1025, 909/1025, 910/1025, 911/1025, 912/1025, 913/1025, 914/1025, 915/1025, 916/1025, 917/1025, 918/1025, 919/1025, 920/1025, 921/1025, 922/1025, 923/1025, 924/1025, 925/1025, 926/1025, 927/1025, 928/1025, 929/1025, 930/1025, 931/1025, 932/1025, 933/1025, 934/1025, 935/1025, 936/1025, 937/1025, 938/1025, 939/1025, 940/1025, 941/1025, 942/1025, 943/1025, 944/1025, 945/1025, 946/1025, 947/1025, 948/1025, 949/1025, 950/1025, 951/1025, 952/1025, 953/1025, 954/1023, 955/1025, 956/1025, 957/1025, 958/1025, 959/1025, 960/1025, 961/1025, 962/1025, 963/1023, 964/1025, 965/1025, 966/1025, 967/1025, 968/1025, 969/1025, 970/1025, 971/1025, 972/1025, 973/1025, 974/1025, 975/1025, 976/1025, 977/1025, 978/1025, 979/1025, 980/1025, 981/1025, 982/1025, 983/1025, 984/1025, 985/1025, 986/1025, 987/1025, 988/1025, 989/1025, 990/1025, 991/1025, 992/1025, 993/1025, 994/1025, 995/1025, 996/1025, 997/1025, 998/1025, 999/1025, 1000/1025, 1001/1025, 1002/1025, 1003/1025, 1004/1025, 1005/1025, 1006/1025, 1007/1025, 1008/1025, 1009/1025, 1010/1025, 1011/1025, 1012/1025, 1013/1025, 1014/1025, 1015/1025, 1016/1025, 1017/1025, 1018/1025, 1019/1025, and 1020/1025.

55. The antibody conjugate of any one of the preceding claims, wherein the antibody comprises three heavy chain CDRs, or variants thereof, and three light chain CDRs, or variants thereof, from a V_{H}/V_{L} pair selected from the group consisting of SEQ ID NOS: 854/1026, 855/1026, 856/1026, 857/1026, 858/1026, 859/1026, 860/1026, 861/1026, 862/1026, 863/1026, 864/1026, 865/1026, 866/1026, 867/1026, 868/1026, 869/1026, 870/1026, 871/1026, 872/1026, 873/1026, 874/1026, 875/1026, 876/1026, 877/1026, 878/1026, 879/1026, 880/1026, 881/1026, 882/1026, 883/1026, 884/1026, 885/1026, 886/1026, 887/1026, 888/1026, 889/1026, 890/1026, 891/1026, 892/1026, 893/1026, 894/1026, 895/1026, 896/1026, 897/1026, 898/1026, 899/1026, 900/1026, 901/1026, 902/1026, 903/1026, 904/1026, 905/1026, 906/1026, 907/1026, 908/1026, 909/1026, 910/1026, 911/1026, 912/1026, 913/1026, 914/1026, 915/1026, 916/1026, 917/1026, 918/1026, 919/1026, 920/1026, 921/1026, 922/1026, 923/1026, 924/1026, 925/1026, 926/1026, 927/1026, 928/1026, 929/1026, 930/1026, 931/1026, 932/1026, 933/1026, 934/1026, 935/1026, 936/1026, 937/1026, 938/1026, 939/1026, 940/1026, 941/1026, 942/1026, 943/1026, 944/1026, 945/1026, 946/1026, 947/1026, 948/1026, 949/1026, 950/1026, 951/1026, 952/1026, 953/1026, 954/1023, 955/1026, 956/1026, 957/1026, 958/1026, 959/1026, 960/1026, 961/1026, 962/1026, 963/1023, 964/1026, 965/1026, 966/1026, 967/1026, 968/1026, 969/1026, 970/1026, 971/1026, 972/1026, 973/1026, 974/1026, 975/1026, 976/1026, 977/1026, 978/1026, 979/1026, 980/1026, 981/1026, 982/1026, 983/1026, 984/1026, 985/1026, 986/1026, 987/1026, 988/1026, 989/1026, 990/1026, 991/1026, 992/1026, 993/1026, 994/1026, 995/1026, 996/1026, 997/1026, 998/1026, 999/1026, 1000/1026, 1001/1026, 1002/1026, 1003/1026, 1004/1026, 1005/1026, 1006/1026, 1007/1026, 1008/1026, 1009/1026, 1010/1026, 1011/1026, 1012/1026, 1013/1026, 1014/1026, 1015/1026, 1016/1026, 1017/1026, 1018/1026, 1019/1026, and 1020/1026.

56. The antibody conjugate of any one of the preceding claims, wherein the antibody comprises a V_{H} selected from SEQ ID NOs: 854-1020.

57. The antibody conjugate of any one of the preceding claims, wherein the antibody comprises a V_{L} selected from SEQ ID NOs: 1021-1026.

58. The antibody conjugate of any one of the preceding claims, wherein the antibody comprises a V_{H}/V_{L} pair, or a variant thereof, selected from the group consisting of SEQ ID NOS: 854/1021, 855/1021, 856/1021, 857/1021, 858/1021, 859/1021, 860/1021, 861/1021, 862/1021, 863/1021, 864/1021, 865/1021, 866/1021, 867/1021, 868/1021, 869/1021, 870/1021, 871/1021, 872/1021, 873/1021, 874/1021, 875/1021, 876/1021, 877/1021, 878/1021, 879/1021, 880/1021, 881/1021, 882/1021, 883/1021, 884/1021, 885/1021, 886/1021, 887/1021, 888/1021, 889/1021, 890/1021, 891/1021, 892/1021, 893/1021, 894/1021, 895/1021, 896/1021, 897/1021, 898/1021, 899/1021, 900/1021, 901/1021, 902/1021, 903/1021, 904/1021, 905/1021, 906/1021, 907/1021, 908/1021, 909/1021, 910/1021, 911/1021, 912/1021, 913/1021, 914/1021, 915/1021, 916/1021, 917/1021, 918/1021, 919/1021, 920/1021, 921/1021, 922/1021, 923/1021, 924/1021, 925/1021, 926/1021, 927/1021, 928/1021, 929/1021, 930/1021, 931/1021, 932/1021, 933/1021, 934/1021, 935/1021, 936/1021, 937/1021, 938/1021, 939/1021, 940/1021, 941/1021, 942/1021, 943/1021, 944/1021, 945/1021, 946/1021, 947/1021, 948/1021, 949/1021, 950/1021, 951/1021, 952/1021, 953/1021, 954/1021, 955/1021, 956/1021, 957/1021, 958/1021, 959/1021, 960/1021, 961/1021, 962/1021, 963/1021, 964/1021, 965/1021, 966/1021, 967/1021, 968/1021, 969/1021, 970/1021, 971/1021, 972/1021, 973/1021, 974/1021, 975/1021, 976/1021, 977/1021, 978/1021, 979/1021, 980/1021, 981/1021, 982/1021, 983/1021, 984/1021, 985/1021, 986/1021, 987/1021, 988/1021, 997/1021, 998/1021, 999/1021, 1000/1021, 1001/1021, 1002/1021, 1003/1021, 1004/1021, 1005/1021, 1006/1021, 1007/1021, 1008/1021, 1009/1021, 1010/1021, 1011/1021, 1012/1021, 1013/1021, 1014/1021, 1015/1021, 1016/1021, 1017/1021, 1018/1021, 1019/1021, and 1020/1021.

59. The antibody conjugate of any one of the preceding claims, wherein the antibody comprises a V_{H}/V_{L} pair, or a variant thereof, selected from the group consisting of SEQ ID NOS: 854/1022, 855/1022, 856/1022, 857/1022, 858/1022, 859/1022, 860/1022, 861/1022, 862/1022, 863/1022, 864/1022, 865/1022, 866/1022, 867/1022, 868/1022, 869/1022, 870/1022, 871/1022, 872/1022, 873/1022, 874/1022, 875/1022, 876/1022, 877/1022, 878/1022, 879/1022, 880/1022, 881/1022, 882/1022, 883/1022, 884/1022, 885/1022, 886/1022, 887/1022, 888/1022, 889/1022, 890/1022, 891/1022, 892/1022, 893/1022, 894/1022, 895/1022, 896/1022, 897/1022, 898/1022, 899/1022, 900/1022, 901/1022, 902/1022, 903/1022, 904/1022, 905/1022, 906/1022, 907/1022, 908/1022, 909/1022, 910/1022, 911/1022, 912/1022, 913/1022, 914/1022, 915/1022, 916/1022, 917/1022, 918/1022, 919/1022, 920/1022, 921/1022, 922/1022, 923/1022, 924/1022, 925/1022, 926/1022, 927/1022, 928/1022, 929/1022, 930/1022, 931/1022, 932/1022, 933/1022, 934/1022, 935/1022, 936/1022, 937/1022, 938/1022, 939/1022, 940/1022, 941/1022, 942/1022, 943/1022, 944/1022, 945/1022, 946/1022, 947/1022, 948/1022, 949/1022, 950/1022, 951/1022, 952/1022, 953/1022, 954/1022, 955/1022, 956/1022, 957/1022, 958/1022, 959/1022, 960/1022, 961/1022, 962/1022, 963/1022, 964/1022, 965/1022, 966/1022, 967/1022, 968/1022, 969/1022, 970/1022, 971/1022, 972/1022, 973/1022, 974/1022, 975/1022, 976/1022, 977/1022, 978/1022, 979/1022, 980/1022, 981/1022, 982/1022, 983/1022, 984/1022, 985/1022, 986/1022, 987/1022, 988/1022, 989/1022, 990/1022, 991/1022, 992/1022, 993/1022, 994/1022, 995/1022, 996/1022, 997/1022, 998/1022, 999/1022, 1000/1022, 1001/1022, 1002/1022, 1003/1022, 1004/1022, 1005/1022, 1006/1022, 1007/1022, 1008/1022, 1009/1022, 1010/1022, 1011/1022, 1012/1022, 1013/1022, 1014/1022, 1015/1022, 1016/1022, 1017/1022, 1018/1022, 1019/1022, and 1020/1022.

60. The antibody conjugate of any one of the preceding claims, wherein the antibody comprises a V_{H}/V_{L} pair, or a variant thereof, selected from the group consisting of SEQ ID NOS: 854/1023, 855/1023, 856/1023, 857/1023, 858/1023, 859/1023, 860/1023, 861/1023, 862/1023, 863/1023, 864/1023, 865/1023, 866/1023, 867/1023, 868/1023, 869/1023, 870/1023, 871/1023, 872/1023, 873/1023, 874/1023, 875/1023, 876/1023, 877/1023, 878/1023, 879/1023, 880/1023, 881/1023, 882/1023, 883/1023, 884/1023, 885/1023, 886/1023, 887/1023, 888/1023, 889/1023, 890/1023, 891/1023, 892/1023, 893/1023, 894/1023, 895/1023, 896/1023, 897/1023, 898/1023, 899/1023, 900/1023, 901/1023, 902/1023, 903/1023, 904/1023, 905/1023, 906/1023, 907/1023, 908/1023, 909/1023, 910/1023, 911/1023, 912/1023, 913/1023, 914/1023, 915/1023, 916/1023, 917/1023, 918/1023, 919/1023, 920/1023, 921/1023, 922/1023, 923/1023, 924/1023, 925/1023, 926/1023, 927/1023, 928/1023, 929/1023, 930/1023, 931/1023, 932/1023, 933/1023, 934/1023, 935/1023, 936/1023, 937/1023, 938/1023, 939/1023, 940/1023, 941/1023, 942/1023, 943/1023, 944/1023, 945/1023, 946/1023, 947/1023, 948/1023, 949/1023, 950/1023, 951/1023, 952/1023, 953/1023, 954/1023, 955/1023, 956/1023, 957/1023, 958/1023, 959/1023, 960/1023, 961/1023, 962/1023, 963/1023, 964/1023, 965/1023, 966/1023, 967/1023, 968/1023, 969/1023, 970/1023, 971/1023, 972/1023, 973/1023, 974/1023, 975/1023, 976/1023, 977/1023, 978/1023, 979/1023, 980/1023, 981/1023, 982/1023, 983/1023, 984/1023, 985/1023, 986/1023, 987/1023, 988/1023, 989/1023, 990/1023, 991/1023, 992/1023, 993/1023, 994/1023, 995/1023, 996/1023, 997/1023, 998/1023, 999/1023, 1000/1023, 1001/1023, 1002/1023, 1003/1023, 1004/1023, 1005/1023, 1006/1023, 1007/1023, 1008/1023, 1009/1023, 1010/1023, 1011/1023, 1012/1023, 1013/1023, 1014/1023, 1015/1023, 1016/1023, 1017/1023, 1018/1023, 1019/1023, and 1020/1023.

61. The antibody conjugate of any one of the preceding claims, wherein the antibody comprises a V_{H}/V_{L} pair, or a variant thereof, selected from the group consisting of SEQ ID NOS: 854/1024, 855/1024, 856/1024, 857/1024, 858/1024, 859/1024, 860/1024, 861/1024, 862/1024, 863/1024, 864/1024, 865/1024, 866/1024, 867/1024, 868/1024, 869/1024, 870/1024, 871/1024, 872/1024, 873/1024, 874/1024, 875/1024, 876/1024, 877/1024, 878/1024, 879/1024, 880/1024, 881/1024, 882/1024, 883/1024, 884/1024, 885/1024, 886/1024, 887/1024, 888/1024, 889/1024, 890/1024, 891/1024, 892/1024, 893/1024, 894/1024, 895/1024, 896/1024, 897/1024, 898/1024, 899/1024, 900/1024, 901/1024, 902/1024, 903/1024, 904/1024, 905/1024, 906/1024, 907/1024, 908/1024, 909/1024, 910/1024, 911/1024, 912/1024, 913/1024, 914/1024, 915/1024, 916/1024, 917/1024, 918/1024, 919/1024, 920/1024, 921/1024, 922/1024, 923/1024, 924/1024, 925/1024, 926/1024, 927/1024, 928/1024, 929/1024, 930/1024, 931/1024, 932/1024, 933/1024, 934/1024, 935/1024, 936/1024, 937/1024, 938/1024, 939/1024, 940/1024, 941/1024, 942/1024, 943/1024, 944/1024, 945/1024, 946/1024, 947/1024, 948/1024, 949/1024, 950/1024, 951/1024, 952/1024, 953/1024, 954/1024, 955/1024, 956/1024, 957/1024, 958/1024, 959/1024, 960/1024, 961/1024, 962/1024, 963/1024, 964/1024, 965/1024, 966/1024, 967/1024, 968/1024, 969/1024, 970/1024, 971/1024, 972/1024, 973/1024, 974/1024, 975/1024, 976/1024, 977/1024, 978/1024, 979/1024, 980/1024, 981/1024, 982/1024, 983/1024, 984/1024, 985/1024, 986/1024, 987/1024, 988/1024, 989/1024, 990/1024, 991/1024, 992/1024, 993/1024, 994/1024, 995/1024, 996/1024, 997/1024, 998/1024, 999/1024, 1000/1024, 1001/1024, 1002/1024, 1003/1024, 1004/1024, 1005/1024, 1006/1024, 1007/1024, 1008/1024, 1009/1024, 1010/1024, 1011/1024, 1012/1024, 1013/1024, 1014/1024, 1015/1024, 1016/1024, 1017/1024, 1018/1024, 1019/1024, and 1020/1024.

62. The antibody conjugate of any one of the preceding claims, wherein the antibody comprises a V_{H}/V_{L} pair, or a variant thereof, selected from the group consisting of SEQ ID NOS: 854/1025, 855/1025, 856/1025, 857/1025, 858/1025, 859/1025, 860/1025, 861/1025, 862/1025, 863/1025, 864/1025, 865/1025, 866/1025, 867/1025, 868/1025, 869/1025, 870/1025, 871/1025, 872/1025, 873/1025, 874/1025, 875/1025, 876/1025, 877/1025, 878/1025, 879/1025, 880/1025, 881/1025, 882/1025, 883/1025, 884/1025, 885/1025, 886/1025, 887/1025, 888/1025, 889/1025, 890/1025, 891/1025, 892/1025, 893/1025, 894/1025, 895/1025, 896/1025, 897/1025, 898/1025, 899/1025, 900/1025, 901/1025, 902/1025, 903/1025, 904/1025, 905/1025, 906/1025, 907/1025, 908/1025, 909/1025, 910/1025, 911/1025, 912/1025, 913/1025, 914/1025, 915/1025, 916/1025, 917/1025, 918/1025, 919/1025, 920/1025, 921/1025, 922/1025, 923/1025, 924/1025, 925/1025, 926/1025, 927/1025, 928/1025, 929/1025, 930/1025, 931/1025, 932/1025, 933/1025, 934/1025, 935/1025, 936/1025, 937/1025, 938/1025, 939/1025, 940/1025, 941/1025, 942/1025, 943/1025, 944/1025, 945/1025, 946/1025, 947/1025, 948/1025, 949/1025, 950/1025, 951/1025, 952/1025, 953/1025, 954/1025, 955/1025, 956/1025, 957/1025, 958/1025, 959/1025, 960/1025, 961/1025, 962/1025, 963/1025, 964/1025, 965/1025, 966/1025, 967/1025, 968/1025, 969/1025, 970/1025, 971/1025, 972/1025, 973/1025, 974/1025, 975/1025, 976/1025, 977/1025, 978/1025, 979/1025, 980/1025, 981/1025, 982/1025, 983/1025, 984/1025, 985/1025, 986/1025, 987/1025, 988/1025, 989/1025, 990/1025, 991/1025, 992/1025, 993/1025, 994/1025, 995/1025, 996/1025, 997/1025, 998/1025, 999/1025, 1000/1025, 1001/1025, 1002/1025, 1003/1025, 1004/1025, 1005/1025, 1006/1025, 1007/1025, 1008/1025, 1009/1025, 1010/1025, 1011/1025, 1012/1025, 1013/1025, 1014/1025, 1015/1025, 1016/1025, 1017/1025, 1018/1025, 1019/1025, and 1020/1025.

63. The antibody conjugate of any one of the preceding claims, wherein the antibody comprises a V_{H}/V_{L} pair, or a variant thereof, selected from the group consisting of SEQ ID NOS: 854/1026, 855/1026, 856/1026, 857/1026, 858/1026, 859/1026, 860/1026, 861/1026, 862/1026, 863/1026, 864/1026, 865/1026, 866/1026, 867/1026, 868/1026, 869/1026, 870/1026, 871/1026, 872/1026, 873/1026, 874/1026, 875/1026, 876/1026, 877/1026, 878/1026, 879/1026, 880/1026, 881/1026, 882/1026, 883/1026, 884/1026, 885/1026, 886/1026, 887/1026, 888/1026, 889/1026, 890/1026, 891/1026, 892/1026, 893/1026, 894/1026, 895/1026, 896/1026, 897/1026, 898/1026, 899/1026, 900/1026, 901/1026, 902/1026, 903/1026, 904/1026, 905/1026, 906/1026, 907/1026, 908/1026, 909/1026, 910/1026, 911/1026, 912/1026, 913/1026, 914/1026, 915/1026, 916/1026, 917/1026, 918/1026, 919/1026, 920/1026, 921/1026, 922/1026, 923/1026, 924/1026, 925/1026, 926/1026, 927/1026, 928/1026, 929/1026, 930/1026, 931/1026, 932/1026, 933/1026, 934/1026, 935/1026, 936/1026, 937/1026, 938/1026, 939/1026, 940/1026, 941/1026, 942/1026, 943/1026, 944/1026, 945/1026, 946/1026, 947/1026, 948/1026, 949/1026, 950/1026, 951/1026, 952/1026, 953/1026, 954/1026, 955/1026, 956/1026, 957/1026, 958/1026, 959/1026, 960/1026, 961/1026, 962/1026, 963/1026, 964/1026, 965/1026, 966/1026, 967/1026, 968/1026, 969/1026, 970/1026, 971/1026, 972/1026, 973/1026, 974/1026, 975/1026, 976/1026, 977/1026, 978/1026, 979/1026, 980/1026, 981/1026, 982/1026, 983/1026, 984/1026, 985/1026, 986/1026, 987/1026, 988/1026, 989/1026, 990/1026, 991/1026, 992/1026, 993/1026, 994/1026, 995/1026, 996/1026, 997/1026, 998/1026, 999/1026, 1000/1026, 1001/1026, 1002/1026, 1003/1026, 1004/1026, 1005/1026, 1006/1026, 1007/1026, 1008/1026, 1009/1026, 1010/1026, 1011/1026, 1012/1026, 1013/1026, 1014/1026, 1015/1026, 1016/1026, 1017/1026, 1018/1026, 1019/1026, and 1020/1026.

64. The antibody conjugate of any one of the preceding claims, wherein the antibody comprises three Chothia HC CDRs according to one of the following groups of HC CDR SEQ ID NOs: 4/338/672; 5/339/673; 6/340/674; 7/341/675; 8/342/676; 9/343/677; 10/344/678; 11/345/679; 12/346/680; 13/347/681; 14/348/682; 15/349/683; 16/350/684; 17/351/685; 18/352/686; 19/353/687; 20/354/688; 21/355/689; 22/356/690; 23/357/691; 24/358/692; 25/359/693; 26/360/694; 27/361/695; 28/362/696; 29/363/697; 30/364/698; 31/365/699; 32/366/700; 33/367/701; 34/368/702; 35/369/703; 36/370/704; 37/371/705; 38/372/706; 39/373/707; 40/374/708; 41/375/709; 42/376/710; 43/377/711; 44/378/712; 45/379/713; 46/380/714; 47/381/715; 48/382/716; 49/383/717; 50/384/718; 51/385/719; 52/386/720; 53/387/721; 54/388/722; 55/389/723; 56/390/724; 57/391/725; 58/392/726; 59/393/727; 60/394/728; 61/395/729; 62/396/730; 63/397/731; 64/398/732; 65/399/733; 66/400/734; 67/401/735; 68/402/736; 69/403/737; 70/404/738; 71/405/739; 72/406/740; 73/407/741; 74/408/742; 75/409/743; 76/410/744; 77/411/745; 78/412/746; 79/413/747; 80/414/748; 81/415/749; 82/416/750; 83/417/751; 84/418/752; 85/419/753; 86/420/754; 87/421/755; 88/422/756; 89/423/757; 90/424/758; 91/425/759; 92/426/760; 93/427/761; 94/428/762; 95/429/763; 96/430/764; 97/431/765; 98/432/766; 99/433/767; 100/434/768; 101/435/769; 102/436/770; 103/437/771; 104/438/772; 105/439/773; 106/440/774; 107/441/775; 108/442/776; 109/443/777; 110/444/778; 111/445/779; 112/446/780; 113/447/781; 114/448/782; 115/449/783; 116/450/784; 117/451/785; 118/452/786; 119/453/787; 120/454/788; 121/455/789; 122/456/790; 123/457/791; 124/458/792; 125/459/793; 126/460/794; 127/461/795; 128/462/796; 129/463/797; 130/464/798; 131/465/799; 132/466/800; 133/467/801; 134/468/802; 135/469/803; 136/470/804; 137/471/805; 138/472/806; 139/473/807; 140/474/808; 141/475/809; 142/476/810; 143/477/811; 144/478/812; 145/479/813; 146/480/814; 147/481/815; 148/482/816; 149/483/817; 150/484/818; 151/485/819; 152/486/820; 153/487/821; 154/488/822; 155/489/823; 156/490/824; 157/491/825; 158/492/826; 159/493/827; 160/494/828; 161/495/829; 162/496/830; 163/497/831; 164/498/832; 165/499/833; 166/500/834; 167/501/835; 168/502/836; 169/503/837; and 170/504/838.

65. The antibody conjugate of any one of the preceding claims, wherein the antibody comprises six CDRs according to one of the following groups of CDR SEQ ID NOS: 4/338/672/839/844/849; 5/339/673/839/844/849; 6/340/674/839/844/849; 7/341/675/839/844/849; 8/342/676/839/844/849; 9/343/677/839/844/849; 10/344/678/839/844/849; 11/345/679/839/844/849; 12/346/680/839/844/849; 13/347/681/839/844/849; 14/348/682/839/844/849; 15/349/683/839/844/849; 16/350/684/839/844/849; 17/351/685/839/844/849; 18/352/686/839/844/849; 19/353/687/839/844/849; 20/354/688/839/844/849; 21/355/689/839/844/849; 22/356/690/839/844/849; 23/357/691/839/844/849; 24/358/692/839/844/849; 25/359/693/839/844/849; 26/360/694/839/844/849; 27/361/695/839/844/849; 28/362/696/839/844/849; 29/363/697/839/844/849; 30/364/698/839/844/849; 31/365/699/839/844/849; 32/366/700/839/844/849; 33/367/701/839/844/849; 34/368/702/839/844/849; 35/369/703/839/844/849; 36/370/704/839/844/849; 37/371/705/839/844/849; 38/372/706/839/844/849; 39/373/707/839/844/849; 40/374/708/839/844/849; 41/375/709/839/844/849; 42/376/710/839/844/849; 43/377/711/839/844/849; 44/378/712/839/844/849; 45/379/713/839/844/849; 46/380/714/839/844/849; 47/381/715/839/844/849; 48/382/716/839/844/849; 49/383/717/839/844/849; 50/384/718/839/844/849; 51/385/719/839/844/849; 52/386/720/839/844/849; 53/387/721/839/844/849; 54/388/722/839/844/849; 55/389/723/839/844/849; 56/390/724/839/844/849; 57/391/725/839/844/849; 58/392/726/839/844/849; 59/393/727/839/844/849; 60/394/728/839/844/849; 61/395/729/839/844/849; 62/396/730/839/844/849; 63/397/731/839/844/849; 64/398/732/839/844/849; 65/399/733/839/844/849; 66/400/734/839/844/849; 67/401/735/839/844/849; 68/402/736/839/844/849; 69/403/737/839/844/849; 70/404/738/839/844/849; 71/405/739/839/844/849; 72/406/740/839/844/849; 73/407/741/839/844/849; 74/408/742/839/844/849; 75/409/743/839/844/849; 76/410/744/839/844/849; 77/411/745/839/844/849; 78/412/746/839/844/849; 79/413/747/839/844/849; 80/414/748/839/844/849; 81/415/749/839/844/849; 82/416/750/839/844/849; 83/417/751/839/844/849; 84/418/752/839/844/849; 85/419/753/839/844/849; 86/420/754/839/844/849; 87/421/755/839/844/849; 88/422/756/839/844/849; 89/423/757/839/844/849; 90/424/758/839/844/849; 91/425/759/839/844/849; 92/426/760/839/844/849; 93/427/761/839/844/849; 94/428/762/839/844/849; 95/429/763/839/844/849; 96/430/764/839/844/849; 97/431/765/839/844/849; 98/432/766/839/844/849; 99/433/767/839/844/849; 100/434/768/839/844/849; 101/435/769/839/844/849; 102/436/770/839/844/849; 103/437/771/839/844/849; 104/438/772/839/844/849; 105/439/773/839/844/849; 106/440/774/839/844/849; 107/441/775/839/844/849; 108/442/776/839/844/849; 109/443/777/839/844/849; 110/444/778/839/844/849; 111/445/779/839/844/849; 112/446/780/839/844/849; 113/447/781/839/844/849; 114/448/782/839/844/849; 115/449/783/839/844/849; 116/450/784/839/844/849; 117/451/785/839/844/849; 118/452/786/839/844/849; 119/453/787/839/844/849; 120/454/788/839/844/849; 121/455/789/839/844/849; 122/456/790/839/844/849; 123/457/791/839/844/849; 124/458/792/839/844/849; 125/459/793/839/844/849; 126/460/794/839/844/849; 127/461/795/839/844/849; 128/462/796/839/844/849; 129/463/797/839/844/849; 130/464/798/839/844/849; 131/465/799/839/844/849; 132/466/800/839/844/849; 133/467/801/839/844/849; 134/468/802/839/844/849; 135/469/803/839/844/849; 136/470/804/839/844/849; 137/471/805/839/844/849; 138/472/806/839/844/849; 139/473/807/839/844/849; 140/474/808/839/844/849; 141/475/809/839/844/849; 142/476/810/839/844/849; 143/477/811/839/844/849; 144/478/812/839/844/849; 145/479/813/840/845/850; 146/480/814/840/845/850; 147/481/815/840/845/850; 148/482/816/840/845/850; 149/483/817/840/845/850; 150/484/818/840/845/850; 151/485/819/840/845/850; 152/486/820/840/845/850; 153/487/821/840/845/850; 154/488/822/840/845/850; 155/489/823/840/845/850; 156/490/824/840/845/850; 157/491/825/840/845/850; 158/492/826/840/845/850; 159/493/827/840/845/850; 160/494/828/840/845/850; 161/495/829/840/845/850; 162/496/830/840/845/850; 163/497/831/840/845/850; 164/498/832/840/845/850; 165/499/833/840/845/850; 166/500/834/840/845/850; 167/501/835/840/845/850; 168/502/836/840/845/850; 169/503/837/840/845/850; 170/504/838/840/845/850; 145/479/813/841/846/851; 146/480/814/841/846/851; 147/481/815/841/846/851; 148/482/816/841/846/851; 149/483/817/841/846/851; 150/484/818/841/846/851; 151/485/819/841/846/851; 152/486/820/841/846/851; 153/487/821/841/846/851; 154/488/822/841/846/851; 155/489/823/841/846/851; 156/490/824/841/846/851; 157/491/825/841/846/851; 158/492/826/841/846/851; 159/493/827/841/846/851; 160/494/828/841/846/851; 161/495/829/841/846/851; 162/496/830/841/846/851; 163/497/831/841/846/851; 164/498/832/841/846/851; 165/499/833/841/846/851; 166/500/834/841/846/851; 167/501/835/841/846/851; 168/502/836/841/846/851; 169/503/837/841/846/851; and 170/504/838/841/846/851.

66. The antibody conjugate of any one of the preceding claims, wherein the antibody comprises three Kabat HC CDRs according to one of the following groups of HC CDR SEQ ID NOs: 171/505/672; 172/506/673; 173/507/674; 174/508/675; 175/509/676; 176/510/677; 177/511/678; 178/512/679; 179/513/680; 180/514/681; 181/515/682; 182/516/683; 183/517/684; 184/518/685; 185/519/686; 186/520/687; 187/521/688; 188/522/689; 189/523/690; 190/524/691; 191/525/692; 192/526/693; 193/527/694; 194/528/695; 195/529/696; 196/530/697; 197/531/698; 198/532/699; 199/533/700; 200/534/701; 201/535/702; 202/536/703; 203/537/704; 204/538/705; 205/539/706; 206/540/707; 207/541/708; 208/542/709; 209/543/710; 210/544/711; 211/545/712; 212/546/713; 213/547/714; 214/548/715; 215/549/716; 216/550/717; 217/551/718; 218/552/719; 219/553/720; 220/554/721; 221/555/722; 222/556/723; 223/557/724; 224/558/725; 225/559/726; 226/560/727; 227/561/728; 228/562/729; 229/563/730; 230/564/731; 231/565/732; 232/566/733; 233/567/734; 234/568/735; 235/569/736; 236/570/737; 237/571/738; 238/572/739; 239/573/740; 240/574/741; 241/575/742; 242/576/743; 243/577/744; 244/578/745; 245/579/746; 246/580/747; 247/581/748; 248/582/749; 249/583/750; 250/584/751; 251/585/752; 252/586/753; 253/587/754; 254/588/755; 255/589/756; 256/590/757; 257/591/758; 258/592/759; 259/593/760; 260/594/761; 261/595/762; 262/596/763; 263/597/764; 264/598/765; 265/599/766; 266/600/767; 267/601/768; 268/602/769; 269/603/770; 270/604/771; 271/605/772; 272/606/773; 273/607/774; 274/608/775; 275/609/776; 276/610/777; 277/611/778; 278/612/779; 279/613/780; 280/614/781; 281/615/782; 282/616/783; 283/617/784; 284/618/785; 285/619/786; 286/620/787; 287/621/788; 288/622/789; 289/623/790; 290/624/791; 291/625/792; 292/626/793; 293/627/794; 294/628/795; 295/629/796; 296/630/797; 297/631/798; 298/632/799; 299/633/800; 300/634/801; 301/635/802; 302/636/803; 303/637/804; 304/638/805; 305/639/806; 306/640/807; 307/641/808; 308/642/809; 309/643/810; 310/644/811; 311/645/812; 312/646/813; 313/647/814; 314/648/815; 315/649/816; 316/650/817; 317/651/818; 318/652/819; 319/653/820; 320/654/821; 321/655/822; 322/656/823; 323/657/824; 324/658/825; 325/659/826; 326/660/827; 327/661/828; 328/662/829; 329/663/830; 330/664/831; 331/665/832; 332/666/833; 333/667/834; 334/668/835; 335/669/836; 336/670/837; and 337/671/838.

67. The antibody conjugate of any one of the preceding claims, wherein the antibody comprises six CDRs according to one of the following groups of CDR SEQ ID NOs: 171/505/672/839/844/849; 172/506/673/839/844/849; 173/507/674/839/844/849; 174/508/675/839/844/849; 175/509/676/839/844/849; 176/510/677/839/844/849; 177/511/678/839/844/849; 178/512/679/839/844/849; 179/513/680/839/844/849; 180/514/681/839/844/849; 181/515/682/839/844/849; 182/516/683/839/844/849; 183/517/684/839/844/849; 184/518/685/839/844/849; 185/519/686/839/844/849; 186/520/687/839/844/849; 187/521/688/839/844/849; 188/522/689/839/844/849; 189/523/690/839/844/849; 190/524/691/839/844/849; 191/525/692/839/844/849; 192/526/693/839/844/849; 193/527/694/839/844/849; 194/528/695/839/844/849; 195/529/696/839/844/849; 196/530/697/839/844/849; 197/531/698/839/844/849; 198/532/699/839/844/849; 199/533/700/839/844/849; 200/534/701/839/844/849; 201/535/702/839/844/849; 202/536/703/839/844/849; 203/537/704/839/844/849; 204/538/705/839/844/849; 205/539/706/839/844/849; 206/540/707/839/844/849; 207/541/708/839/844/849; 208/542/709/839/844/849; 209/543/710/839/844/849; 210/544/711/839/844/849; 211/545/712/839/844/849; 212/546/713/839/844/849; 213/547/714/839/844/849; 214/548/715/839/844/849; 215/549/716/839/844/849; 216/550/717/839/844/849; 217/551/718/839/844/849; 218/552/719/839/844/849; 219/553/720/839/844/849; 220/554/721/839/844/849; 221/555/722/839/844/849; 222/556/723/839/844/849; 223/557/724/839/844/849; 224/558/725/839/844/849; 225/559/726/839/844/849; 226/560/727/839/844/849; 227/561/728/839/844/849; 228/562/729/839/844/849; 229/563/730/839/844/849; 230/564/731/839/844/849; 231/565/732/839/844/849; 232/566/733/839/844/849; 233/567/734/839/844/849; 234/568/735/839/844/849; 235/569/736/839/844/849; 236/570/737/839/844/849; 237/571/738/839/844/849; 238/572/739/839/844/849; 239/573/740/839/844/849; 240/574/741/839/844/849; 241/575/742/839/844/849; 242/576/743/839/844/849; 243/577/744/839/844/849; 244/578/745/839/844/849; 245/579/746/839/844/849; 246/580/747/839/844/849; 247/581/748/839/844/849; 248/582/749/839/844/849; 249/583/750/839/844/849; 250/584/751/839/844/849; 251/585/752/839/844/849; 252/586/753/839/844/849; 253/587/754/839/844/849; 254/588/755/839/844/849; 255/589/756/839/844/849; 256/590/757/839/844/849; 257/591/758/839/844/849; 258/592/759/839/844/849; 259/593/760/839/844/849; 260/594/761/839/844/849; 261/595/762/839/844/849; 262/596/763/839/844/849; 263/597/764/839/844/849; 264/598/765/839/844/849; 265/599/766/839/844/849; 266/600/767/839/844/849; 267/601/768/839/844/849; 268/602/769/839/844/849; 269/603/770/839/844/849; 270/604/771/839/844/849; 271/605/772/839/844/849; 272/606/773/839/844/849; 273/607/774/839/844/849; 274/608/775/839/844/849; 275/609/776/839/844/849; 276/610/777/839/844/849; 277/611/778/839/844/849; 278/612/779/839/844/849; 279/613/780/839/844/849; 280/614/781/839/844/849; 281/615/782/839/844/849; 282/616/783/839/844/849; 283/617/784/839/844/849; 284/618/785/839/844/849; 285/619/786/839/844/849; 286/620/787/839/844/849; 287/621/788/839/844/849; 288/622/789/839/844/849; 289/623/790/839/844/849; 290/624/791/839/844/849; 291/625/792/839/844/849; 292/626/793/839/844/849; 293/627/794/839/844/849; 294/628/795/839/844/849; 295/629/796/839/844/849; 296/630/797/839/844/849; 297/631/798/839/844/849; 298/632/799/839/844/849; 299/633/800/839/844/849; 300/634/801/839/844/849; 301/635/802/839/844/849; 302/636/803/839/844/849; 303/637/804/839/844/849; 304/638/805/839/844/849; 305/639/806/839/844/849; 306/640/807/839/844/849; 307/641/808/839/844/849; 308/642/809/839/844/849; 309/643/810/839/844/849; 310/644/811/839/844/849; 311/645/812/840/845/850; 312/646/813/840/845/850; 313/647/814/840/845/850; 314/648/815/840/845/850; 315/649/816/840/845/850; 316/650/817/840/845/850; 317/651/818/840/845/850; 318/652/819/840/845/850; 319/653/820/840/845/850; 320/654/821/840/845/850; 321/655/822/840/845/850; 322/656/823/840/845/850; 323/657/824/840/845/850; 324/658/825/840/845/850; 325/659/826/840/845/850; 326/660/827/840/845/850; 327/661/828/840/845/850; 328/662/829/840/845/850; 329/663/830/840/845/850; 330/664/831/840/845/850; 331/665/832/840/845/850; 332/666/833/840/845/850; 333/667/834/840/845/850; 334/668/835/840/845/850; 335/669/836/840/845/850; 336/670/837/840/845/850; 337/671/838/840/845/850; 311/645/812/841/846/851; 312/646/813/841/846/851; 313/647/814/841/846/851; 314/648/815/841/846/851; 315/649/816/841/846/851; 316/650/817/841/846/851; 317/651/818/841/846/851; 318/652/819/841/846/851; 319/653/820/841/846/851; 320/654/821/841/846/851; 321/655/822/841/846/851; 322/656/823/841/846/851; 323/657/824/841/846/851; 324/658/825/841/846/851; 325/659/826/841/846/851; 326/660/827/841/846/851; 327/661/828/841/846/851; 328/662/829/841/846/851; 329/663/830/841/846/851; 330/664/831/841/846/851; 331/665/832/841/846/851; 332/666/833/841/846/851; 333/667/834/841/846/851; 334/668/835/841/846/851; 335/669/836/841/846/851; 336/670/837/841/846/851; and 337/671/838/841/846/851.

68. The antibody conjugate of any one of the preceding claims, wherein the antibody comprises:
(a) three heavy chain CDRs and three light chain CDRs of the V_{H} region SEQ ID NO: 854, and the V_{L} region SEQ ID NO: 1021;
(b) three heavy chain CDRs and three light chain CDRs of the V_{H} region SEQ ID NO: 859, and the V_{L} region SEQ ID NO: 1021; or
(c) three heavy chain CDRs and three light chain CDRs of the V_{H} region SEQ ID NO: 895, and the V_{L} region SEQ ID NO: 1021.

69. The antibody conjugate of any one of the preceding claims, wherein the antibody comprises three heavy chain CDRs and three light chain CDRs of the V_{H} region SEQ ID NO: 854, and the V_{L} region SEQ ID NO: 1021.

70. The antibody conjugate of any one of the preceding claims, wherein the antibody comprises:
(a) a V_{H} comprising: a CDR-H1 comprising at least one of SEQ ID NOs: 4 and 171; a CDR-H2 comprising at least one of SEQ ID NOs: 338 and 505; and a CDR-H3 comprising SEQ ID NO: 672; and a V_{L} comprising: a CDR-L1 comprising SEQ ID NO: 839; a CDR-L2 comprising SEQ ID NO: 844; and a CDR-L3 comprising SEQ ID NO: 849;
(b) a V_{H} comprising: a CDR-H1 comprising at least one of SEQ ID NOs: 9 and 176; a CDR-H2 comprising at least one of SEQ ID NOs: 343 and 510; and a CDR-H3 comprising SEQ ID NO: 677; and a V_{L} comprising: a CDR-L1 comprising SEQ ID NO: 839; a CDR-L2 comprising SEQ ID NO: 844; and a CDR-L3 comprising SEQ ID NO: 849; or
(c) a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849.

71. The antibody conjugate any of claims 49-70, wherein the variant thereof has 20, 15, 10, 9, 8, 7, 6, 5, 4, 3, 2, or 1 amino acid substitutions.

72. The antibody conjugate of claim 71, wherein the amino acid substitutions are conservative amino acid substitutions.

73. The antibody conjugate of any of claims 49-72, wherein the variant thereof has 90%, 95%, 96%, 97%, 98%, or 99% sequence identity.

74. The antibody conjugate of any one of the preceding claims, wherein the antibody conjugate comprises:
(a) an antibody comprising a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849;

75. The antibody conjugate of any one of the preceding claims, wherein the antibody conjugate comprises:
(a) an antibody comprising a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849;
(b) para-azidomethylphenylalanine residues at antibody sites HC180 and HC404; and
(c) the following linker-payload structure, wherein n2 is 4 and each linker-payload is bonded to one of the para-azidomethylphenylalanine residue side chains:

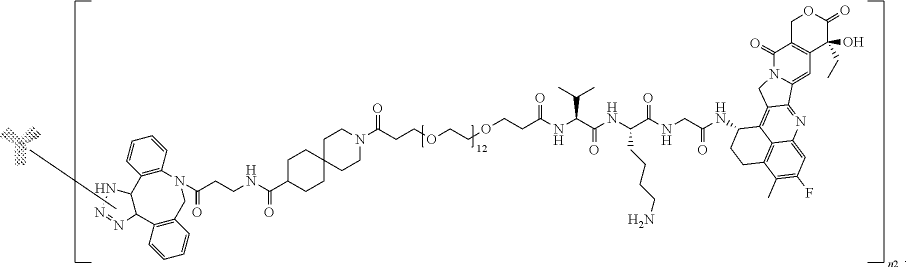

76. The antibody conjugate of any one of the preceding claims, wherein the antibody conjugate comprises:
(a) an antibody comprising a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849;
(b) para-azidomethylphenylalanine residues at antibody sites HC180, HC241, HC404, and LC42; and
(c) the following linker-payload structure, wherein n2 is 8 and each linker-payload is bonded to one of the para-azidomethylphenylalanine residue side chains:

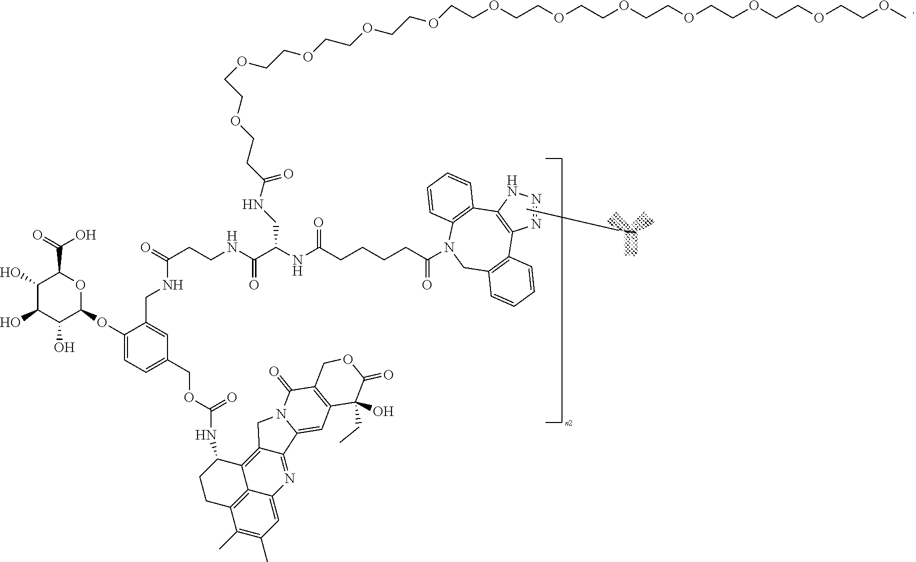

77. The antibody conjugate of any one of the preceding claims, wherein the antibody conjugate comprises:
(a) an antibody comprising a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849;
(b) para-azidomethylphenylalanine residues at antibody sites HC180, HC241, HC404, and LC42; and
(c) the following linker-payload structure, wherein n2 is 8 and each linker-payload is bonded to one of the para-azidomethylphenylalanine residue side chains:

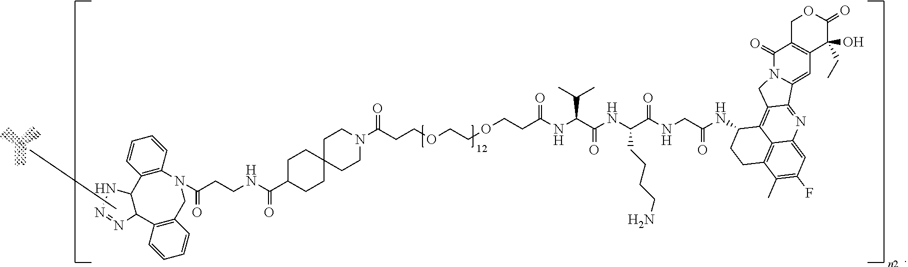

78. The antibody conjugate of any one of the preceding claims, wherein the antibody conjugate comprises:
(a) an antibody comprising a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849;
(b) para-azidomethylphenylalanine residues at antibody sites HC180, HC241, HC404, and LC42; and
(c) the following linker-payload structure, wherein n2 is 8 and each linker-payload is bonded to one of the para-azidomethylphenylalanine residue side chains:

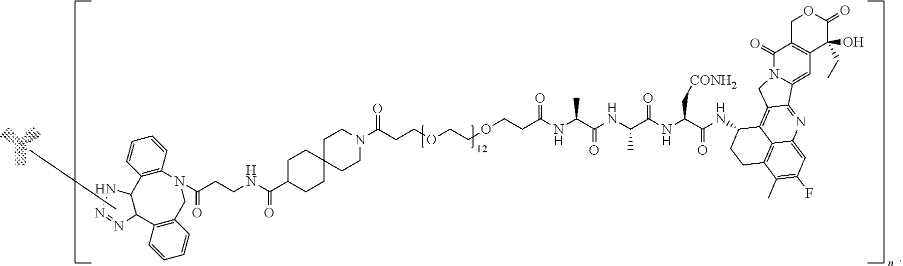

79. The antibody conjugate of any one of the preceding claims, wherein the antibody conjugate comprises:
(a) an antibody comprising a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849;
(b) para-azidomethylphenylalanine residues at antibody sites HC180, HC241, and HC404; and
(c) the following linker-payload structure, wherein n2 is 8 and each linker-payload is bonded to one of the para-azidomethylphenylalanine residue side chains:

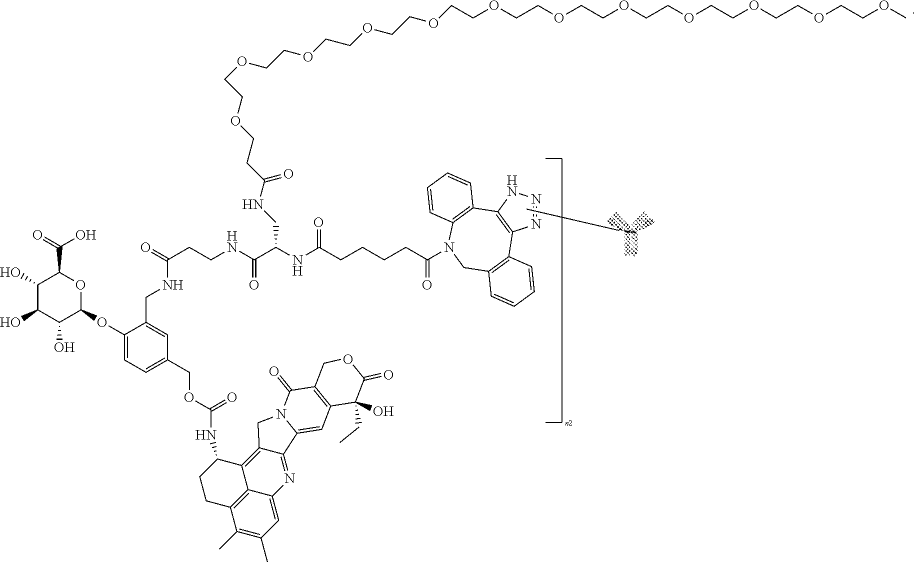

80. The antibody conjugate of any of claims 74-79, wherein the antibody comprises the V_{H}/V_{L} pair SEQ ID NOs: 895/1021.

81. The antibody conjugate of any one of the preceding claims, wherein the antibody binds to the same epitope as a second antibody with a V_{H} region selected from SEQ ID NOs: 854-1020 and a V_{L} region selected from SEQ ID NOs: 1021-1026.

82. The antibody conjugate of claim 81, wherein the antibody inhibits binding of the second antibody to ROR1 by at least 50%, or wherein the second antibody inhibits binding of the isolated antibody to ROR1 by at least 50%.

83. The antibody conjugate of any one of the preceding claims, wherein the antibody has a kₐ of about 9.17×10⁴ M⁻¹×sec⁻¹ to about 5.94×10⁶ M⁻¹×sec⁻¹ when associating with human ROR1 at a temperature of 25° C.

84. The antibody conjugate of any one of the preceding claims, wherein the antibody has a kₐ of about 1.01×10⁴ sec⁻¹ to about 0.05 sec⁻¹ when dissociating from human ROR1 at a temperature of 25° C.

85. The antibody conjugate of any one of the preceding claims, wherein the antibody has a K_{D} of about 1.39×10⁻¹⁰ M to about 1.01×10⁻⁷ M when bound to human ROR1 at a temperature of 25° C.

86. The antibody conjugate of any one of the preceding claims, wherein the antibody comprises at least one constant region domain.

87. The antibody conjugate of claim 86, wherein the constant region comprises a sequence selected from SEQ ID NOs: 1027-1029.

88. The antibody conjugate of any of the preceding claims, wherein the antibody is a monoclonal antibody.

89. The antibody conjugate of any of the preceding claims, wherein the antibody is an IgA, an IgD, an IgE, an IgG, or an IgM.

90. The antibody conjugate of any of the preceding claims, wherein the antibody is humanized or human.

91. The antibody conjugate of any of the preceding claims, wherein the antibody is glycosylated.

92. The antibody conjugate of any of the preceding claims, wherein the antibody is an antibody fragment.

93. The antibody conjugate of claim 92, wherein the antibody fragment is selected from an Fv fragment, a Fab fragment, a F(ab′)₂ fragment, a Fab′ fragment, an scFv (sFv) fragment, and an scFv-Fc fragment.

94. The antibody conjugate of claim 92, wherein the antibody is an scFv fragment.

95. An isolated antibody that specifically binds to ROR1, wherein the antibody comprises three heavy chain CDRs from a V_{H} sequence selected from SEQ ID NOs: 854-1020, or variants thereof, and three light chain CDRs from a V_{L} sequence selected from SEQ ID NOs: 1021-1026, or variants thereof.

96. The isolated antibody of claim 95, wherein the antibody comprises three heavy chain CDRs, or variants thereof, and three light chain CDRs, or variants thereof, from a V_{H}/V_{L} pair selected from the group consisting of SEQ ID NOS: 854/1021, 855/1021, 856/1021, 857/1021, 858/1021, 859/1021, 860/1021, 861/1021, 862/1021, 863/1021, 864/1021, 865/1021, 866/1021, 867/1021, 868/1021, 869/1021, 870/1021, 871/1021, 872/1021, 873/1021, 874/1021, 875/1021, 876/1021, 877/1021, 878/1021, 879/1021, 880/1021, 881/1021, 882/1021, 883/1021, 884/1021, 885/1021, 886/1021, 887/1021, 888/1021, 889/1021, 890/1021, 891/1021, 892/1021, 893/1021, 894/1021, 895/1021, 896/1021, 897/1021, 898/1021, 899/1021, 900/1021, 901/1021, 902/1021, 903/1021, 904/1021, 905/1021, 906/1021, 907/1021, 908/1021, 909/1021, 910/1021, 911/1021, 912/1021, 913/1021, 914/1021, 915/1021, 916/1021, 917/1021, 918/1021, 919/1021, 920/1021, 921/1021, 922/1021, 923/1021, 924/1021, 925/1021, 926/1021, 927/1021, 928/1021, 929/1021, 930/1021, 931/1021, 932/1021, 933/1021, 934/1021, 935/1021, 936/1021, 937/1021, 938/1021, 939/1021, 940/1021, 941/1021, 942/1021, 943/1021, 944/1021, 945/1021, 946/1021, 947/1021, 948/1021, 949/1021, 950/1021, 951/1021, 952/1021, 953/1021, 954/1021, 955/1021, 956/1021, 957/1021, 958/1021, 959/1021, 960/1021, 961/1021, 962/1021, 963/1021, 964/1021, 965/1021, 966/1021, 967/1021, 968/1021, 969/1021, 970/1021, 971/1021, 972/1021, 973/1021, 974/1021, 975/1021, 976/1021, 977/1021, 978/1021, 979/1021, 980/1021, 981/1021, 982/1021, 983/1021, 984/1021, 985/1021, 986/1021, 987/1021, 988/1021, 989/1021, 990/1021, 991/1021, 992/1021, 993/1021, 994/1021, 995/1021, 996/1021, 997/1021, 998/1021, 999/1021, 1000/1021, 1001/1021, 1002/1021, 1003/1021, 1004/1021, 1005/1021, 1006/1021, 1007/1021, 1008/1021, 1009/1021, 1010/1021, 1011/1021, 1012/1021, 1013/1021, 1014/1021, 1015/1021, 1016/1021, 1017/1021, 1018/1021, 1019/1021, and 1020/1021.

97. The isolated antibody of claim 95, wherein the antibody comprises three heavy chain CDRs, or variants thereof, and three light chain CDRs, or variants thereof, from a V_{H}/V_{L} pair selected from the group consisting of SEQ ID NOS: 854/1022, 855/1022, 856/1022, 857/1022, 858/1022, 859/1022, 860/1022, 861/1022, 862/1022, 863/1022, 864/1022, 865/1022, 866/1022, 867/1022, 868/1022, 869/1022, 870/1022, 871/1022, 872/1022, 873/1022, 874/1022, 875/1022, 876/1022, 877/1022, 878/1022, 879/1022, 880/1022, 881/1022, 882/1022, 883/1022, 884/1022, 885/1022, 886/1022, 887/1022, 888/1022, 889/1022, 890/1022, 891/1022, 892/1022, 893/1022, 894/1022, 895/1022, 896/1022, 897/1022, 898/1022, 899/1022, 900/1022, 901/1022, 902/1022, 903/1022, 904/1022, 905/1022, 906/1022, 907/1022, 908/1022, 909/1022, 910/1022, 911/1022, 912/1022, 913/1022, 914/1022, 915/1022, 916/1022, 917/1022, 918/1022, 919/1022, 920/1022, 921/1022, 922/1022, 923/1022, 924/1022, 925/1022, 926/1022, 927/1022, 928/1022, 929/1022, 930/1022, 931/1022, 932/1022, 933/1022, 934/1022, 935/1022, 936/1022, 937/1022, 938/1022, 939/1022, 940/1022, 941/1022, 942/1022, 943/1022, 944/1022, 945/1022, 946/1022, 947/1022, 948/1022, 949/1022, 950/1022, 951/1022, 952/1022, 953/1022, 954/1022, 955/1022, 956/1022, 957/1022, 958/1022, 959/1022, 960/1022, 961/1022, 962/1022, 963/1022, 964/1022, 965/1022, 966/1022, 967/1022, 968/1022, 969/1022, 970/1022, 971/1022, 972/1022, 973/1022, 974/1022, 975/1022, 976/1022, 977/1022, 978/1022, 979/1022, 980/1022, 981/1022, 982/1022, 983/1022, 984/1022, 985/1022, 986/1022, 987/1022, 988/1022, 989/1022, 990/1022, 991/1022, 992/1022, 993/1022, 994/1022, 995/1022, 996/1022, 997/1022, 998/1022, 999/1022, 1000/1022, 1001/1022, 1002/1022, 1003/1022, 1004/1022, 1005/1022, 1006/1022, 1007/1022, 1008/1022, 1009/1022, 1010/1022, 1011/1022, 1012/1022, 1013/1022, 1014/1022, 1015/1022, 1016/1022, 1017/1022, 1018/1022, 1019/1022, and 1020/1022.

98. The isolated antibody of claim 95, wherein the antibody comprises three heavy chain CDRs, or variants thereof, and three light chain CDRs, or variants thereof, from a V_{H}/V_{L} pair selected from the group consisting of SEQ ID NOS: 854/1023, 855/1023, 856/1023, 857/1023, 858/1023, 859/1023, 860/1023, 861/1023, 862/1023, 863/1023, 864/1023, 865/1023, 866/1023, 867/1023, 868/1023, 869/1023, 870/1023, 871/1023, 872/1023, 873/1023, 874/1023, 875/1023, 876/1023, 877/1023, 878/1023, 879/1023, 880/1023, 881/1023, 882/1023, 883/1023, 884/1023, 885/1023, 886/1023, 887/1023, 888/1023, 889/1023, 890/1023, 891/1023, 892/1023, 893/1023, 894/1023, 895/1023, 896/1023, 897/1023, 898/1023, 899/1023, 900/1023, 901/1023, 902/1023, 903/1023, 904/1023, 905/1023, 906/1023, 907/1023, 908/1023, 909/1023, 910/1023, 911/1023, 912/1023, 913/1023, 914/1023, 915/1023, 916/1023, 917/1023, 918/1023, 919/1023, 920/1023, 921/1023, 922/1023, 923/1023, 924/1023, 925/1023, 926/1023, 927/1023, 928/1023, 929/1023, 930/1023, 931/1023, 932/1023, 933/1023, 934/1023, 935/1023, 936/1023, 937/1023, 938/1023, 939/1023, 940/1023, 941/1023, 942/1023, 943/1023, 944/1023, 945/1023, 946/1023, 947/1023, 948/1023, 949/1023, 950/1023, 951/1023, 952/1023, 953/1023, 954/1023, 955/1023, 956/1023, 957/1023, 958/1023, 959/1023, 960/1023, 961/1023, 962/1023, 963/1023, 964/1023, 965/1023, 966/1023, 967/1023, 968/1023, 969/1023, 970/1023, 971/1023, 972/1023, 973/1023, 974/1023, 975/1023, 976/1023, 977/1023, 978/1023, 979/1023, 980/1023, 981/1023, 982/1023, 983/1023, 984/1023, 985/1023, 986/1023, 987/1023, 988/1023, 989/1023, 990/1023, 991/1023, 992/1023, 993/1023, 994/1023, 995/1023, 996/1023, 997/1023, 998/1023, 999/1023, 1000/1023, 1001/1023, 1002/1023, 1003/1023, 1004/1023, 1005/1023, 1006/1023, 1007/1023, 1008/1023, 1009/1023, 1010/1023, 1011/1023, 1012/1023, 1013/1023, 1014/1023, 1015/1023, 1016/1023, 1017/1023, 1018/1023, 1019/1023, and 1020/1023.

99. The isolated antibody of claim 95, wherein the antibody comprises three heavy chain CDRs, or variants thereof, and three light chain CDRs, or variants thereof, from a V_{H}/V_{L} pair selected from the group consisting of SEQ ID NOS: 854/1024, 855/1024, 856/1024, 857/1024, 858/1024, 859/1024, 860/1024, 861/1024, 862/1024, 863/1024, 864/1024, 865/1024, 866/1024, 867/1024, 868/1024, 869/1024, 870/1024, 871/1024, 872/1024, 873/1024, 874/1024, 875/1024, 876/1024, 877/1024, 878/1024, 879/1024, 880/1024, 881/1024, 882/1024, 883/1024, 884/1024, 885/1024, 886/1024, 887/1024, 888/1024, 889/1024, 890/1024, 891/1024, 892/1024, 893/1024, 894/1024, 895/1024, 896/1024, 897/1024, 898/1024, 899/1024, 900/1024, 901/1024, 902/1024, 903/1024, 904/1024, 905/1024, 906/1024, 907/1024, 908/1024, 909/1024, 910/1024, 911/1024, 912/1024, 913/1024, 914/1024, 915/1024, 916/1024, 917/1024, 918/1024, 919/1024, 920/1024, 921/1024, 922/1024, 923/1024, 924/1024, 925/1024, 926/1024, 927/1024, 928/1024, 929/1024, 930/1024, 931/1024, 932/1024, 933/1024, 934/1024, 935/1024, 936/1024, 937/1024, 938/1024, 939/1024, 940/1024, 941/1024, 942/1024, 943/1024, 944/1024, 945/1024, 946/1024, 947/1024, 948/1024, 949/1024, 950/1024, 951/1024, 952/1024, 953/1024, 954/1024, 955/1024, 956/1024, 957/1024, 958/1024, 959/1024, 960/1024, 961/1024, 962/1024, 963/1024, 964/1024, 965/1024, 966/1024, 967/1024, 968/1024, 969/1024, 970/1024, 971/1024, 972/1024, 973/1024, 974/1024, 975/1024, 976/1024, 977/1024, 978/1024, 979/1024, 980/1024, 981/1024, 982/1024, 983/1024, 984/1024, 985/1024, 986/1024, 987/1024, 988/1024, 989/1024, 990/1024, 991/1024, 992/1024, 993/1024, 994/1024, 995/1024, 996/1024, 997/1024, 998/1024, 999/1024, 1000/1024, 1001/1024, 1002/1024, 1003/1024, 1004/1024, 1005/1024, 1006/1024, 1007/1024, 1008/1024, 1009/1024, 1010/1024, 1011/1024, 1012/1024, 1013/1024, 1014/1024, 1015/1024, 1016/1024, 1017/1024, 1018/1024, 1019/1024, and 1020/1024.

100. The isolated antibody of claim 95, wherein the antibody comprises three heavy chain CDRs, or variants thereof, and three light chain CDRs, or variants thereof, from a V_{H}/V_{L} pair selected from the group consisting of SEQ ID NOS: 854/1025, 855/1025, 856/1025, 857/1025, 858/1025, 859/1025, 860/1025, 861/1025, 862/1025, 863/1025, 864/1025, 865/1025, 866/1025, 867/1025, 868/1025, 869/1025, 870/1025, 871/1025, 872/1025, 873/1025, 874/1025, 875/1025, 876/1025, 877/1025, 878/1025, 879/1025, 880/1025, 881/1025, 882/1025, 883/1025, 884/1025, 885/1025, 886/1025, 887/1025, 888/1025, 889/1025, 890/1025, 891/1025, 892/1025, 893/1025, 894/1025, 895/1025, 896/1025, 897/1025, 898/1025, 899/1025, 900/1025, 901/1025, 902/1025, 903/1025, 904/1025, 905/1025, 906/1025, 907/1025, 908/1025, 909/1025, 910/1025, 911/1025, 912/1025, 913/1025, 914/1025, 915/1025, 916/1025, 917/1025, 918/1025, 919/1025, 920/1025, 921/1025, 922/1025, 923/1025, 924/1025, 925/1025, 926/1025, 927/1025, 928/1025, 929/1025, 930/1025, 931/1025, 932/1025, 933/1025, 934/1025, 935/1025, 936/1025, 937/1025, 938/1025, 939/1025, 940/1025, 941/1025, 942/1025, 943/1025, 944/1025, 945/1025, 946/1025, 947/1025, 948/1025, 949/1025, 950/1025, 951/1025, 952/1025, 953/1025, 954/1025, 955/1025, 956/1025, 957/1025, 958/1025, 959/1025, 960/1025, 961/1025, 962/1025, 963/1025, 964/1025, 965/1025, 966/1025, 967/1025, 968/1025, 969/1025, 970/1025, 971/1025, 972/1025, 973/1025, 974/1025, 975/1025, 976/1025, 977/1025, 978/1025, 979/1025, 980/1025, 981/1025, 982/1025, 983/1025, 984/1025, 985/1025, 986/1025, 987/1025, 988/1025, 989/1025, 990/1025, 991/1025, 992/1025, 993/1025, 994/1025, 995/1025, 996/1025, 997/1025, 998/1025, 999/1025, 1000/1025, 1001/1025, 1002/1025, 1003/1025, 1004/1025, 1005/1025, 1006/1025, 1007/1025, 1008/1025, 1009/1025, 1010/1025, 1011/1025, 1012/1025, 1013/1025, 1014/1025, 1015/1025, 1016/1025, 1017/1025, 1018/1025, 1019/1025, and 1020/1025.

101. The isolated antibody of claim 95, wherein the antibody comprises three heavy chain CDRs, or variants thereof, and three light chain CDRs, or variants thereof, from a V_{H}/V_{L} pair selected from the group consisting of SEQ ID NOS: 854/1026, 855/1026, 856/1026, 857/1026, 858/1026, 859/1026, 860/1026, 861/1026, 862/1026, 863/1026, 864/1026, 865/1026, 866/1026, 867/1026, 868/1026, 869/1026, 870/1026, 871/1026, 872/1026, 873/1026, 874/1026, 875/1026, 876/1026, 877/1026, 878/1026, 879/1026, 880/1026, 881/1026, 882/1026, 883/1026, 884/1026, 885/1026, 886/1026, 887/1026, 888/1026, 889/1026, 890/1026, 891/1026, 892/1026, 893/1026, 894/1026, 895/1026, 896/1026, 897/1026, 898/1026, 899/1026, 900/1026, 901/1026, 902/1026, 903/1026, 904/1026, 905/1026, 906/1026, 907/1026, 908/1026, 909/1026, 910/1026, 911/1026, 912/1026, 913/1026, 914/1026, 915/1026, 916/1026, 917/1026, 918/1026, 919/1026, 920/1026, 921/1026, 922/1026, 923/1026, 924/1026, 925/1026, 926/1026, 927/1026, 928/1026, 929/1026, 930/1026, 931/1026, 932/1026, 933/1026, 934/1026, 935/1026, 936/1026, 937/1026, 938/1026, 939/1026, 940/1026, 941/1026, 942/1026, 943/1026, 944/1026, 945/1026, 946/1026, 947/1026, 948/1026, 949/1026, 950/1026, 951/1026, 952/1026, 953/1026, 954/1026, 955/1026, 956/1026, 957/1026, 958/1026, 959/1026, 960/1026, 961/1026, 962/1026, 963/1026, 964/1026, 965/1026, 966/1026, 967/1026, 968/1026, 969/1026, 970/1026, 971/1026, 972/1026, 973/1026, 974/1026, 975/1026, 976/1026, 977/1026, 978/1026, 979/1026, 980/1026, 981/1026, 982/1026, 983/1026, 984/1026, 985/1026, 986/1026, 987/1026, 988/1026, 989/1026, 990/1026, 991/1026, 992/1026, 993/1026, 994/1026, 995/1026, 996/1026, 997/1026, 998/1026, 999/1026, 1000/1026, 1001/1026, 1002/1026, 1003/1026, 1004/1026, 1005/1026, 1006/1026, 1007/1026, 1008/1026, 1009/1026, 1010/1026, 1011/1026, 1012/1026, 1013/1026, 1014/1026, 1015/1026, 1016/1026, 1017/1026, 1018/1026, 1019/1026, and 1020/1026.

102. The isolated antibody of claim 95, wherein the antibody comprises a V_{H} selected from SEQ ID NOs: 854-1020.

103. The isolated antibody of claim 95, wherein the antibody comprises a V_{L} selected from SEQ ID NOs: 1021-1026.

104. The isolated antibody of claim 95, wherein the antibody comprises a V_{H}/V_{L} pair, or a variant thereof, selected from the group consisting of SEQ ID NOS: 854/1021, 855/1021, 856/1021, 857/1021, 858/1021, 859/1021, 860/1021, 861/1021, 862/1021, 863/1021, 864/1021, 865/1021, 866/1021, 867/1021, 868/1021, 869/1021, 870/1021, 871/1021, 872/1021, 873/1021, 874/1021, 875/1021, 876/1021, 877/1021, 878/1021, 879/1021, 880/1021, 881/1021, 882/1021, 883/1021, 884/1021, 885/1021, 886/1021, 887/1021, 888/1021, 889/1021, 890/1021, 891/1021, 892/1021, 893/1021, 894/1021, 895/1021, 896/1021, 897/1021, 898/1021, 899/1021, 900/1021, 901/1021, 902/1021, 903/1021, 904/1021, 905/1021, 906/1021, 907/1021, 908/1021, 909/1021, 910/1021, 911/1021, 912/1021, 913/1021, 914/1021, 915/1021, 916/1021, 917/1021, 918/1021, 919/1021, 920/1021, 921/1021, 922/1021, 923/1021, 924/1021, 925/1021, 926/1021, 927/1021, 928/1021, 929/1021, 930/1021, 931/1021, 932/1021, 933/1021, 934/1021, 935/1021, 936/1021, 937/1021, 938/1021, 939/1021, 940/1021, 941/1021, 942/1021, 943/1021, 944/1021, 945/1021, 946/1021, 947/1021, 948/1021, 949/1021, 950/1021, 951/1021, 952/1021, 953/1021, 954/1021, 955/1021, 956/1021, 957/1021, 958/1021, 959/1021, 960/1021, 961/1021, 962/1021, 963/1021, 964/1021, 965/1021, 966/1021, 967/1021, 968/1021, 969/1021, 970/1021, 971/1021, 972/1021, 973/1021, 974/1021, 975/1021, 976/1021, 977/1021, 978/1021, 979/1021, 980/1021, 981/1021, 982/1021, 983/1021, 984/1021, 985/1021, 986/1021, 987/1021, 988/1021, 989/1021, 990/1021, 991/1021, 992/1021, 993/1021, 994/1021, 995/1021, 996/1021, 997/1021, 998/1021, 999/1021, 1000/1021, 1001/1021, 1002/1021, 1003/1021, 1004/1021, 1005/1021, 1006/1021, 1007/1021, 1008/1021, 1009/1021, 1010/1021, 1011/1021, 1012/1021, 1013/1021, 1014/1021, 1015/1021, 1016/1021, 1017/1021, 1018/1021, 1019/1021, and 1020/1021.

105. The isolated antibody of claim 95, wherein the antibody comprises a V_{H}/V_{L} pair, or a variant thereof, selected from the group consisting of SEQ ID NOS: 854/1022, 855/1022, 856/1022, 857/1022, 858/1022, 859/1022, 860/1022, 861/1022, 862/1022, 863/1022, 864/1022, 865/1022, 866/1022, 867/1022, 868/1022, 869/1022, 870/1022, 871/1022, 872/1022, 873/1022, 874/1022, 875/1022, 876/1022, 877/1022, 878/1022, 879/1022, 880/1022, 881/1022, 882/1022, 883/1022, 884/1022, 885/1022, 886/1022, 887/1022, 888/1022, 889/1022, 890/1022, 891/1022, 892/1022, 893/1022, 894/1022, 895/1022, 896/1022, 897/1022, 898/1022, 899/1022, 900/1022, 901/1022, 902/1022, 903/1022, 904/1022, 905/1022, 906/1022, 907/1022, 908/1022, 909/1022, 910/1022, 911/1022, 912/1022, 913/1022, 914/1022, 915/1022, 916/1022, 917/1022, 918/1022, 919/1022, 920/1022, 921/1022, 922/1022, 923/1022, 924/1022, 925/1022, 926/1022, 927/1022, 928/1022, 929/1022, 930/1022, 931/1022, 932/1022, 933/1022, 934/1022, 935/1022, 936/1022, 937/1022, 938/1022, 939/1022, 940/1022, 941/1022, 942/1022, 943/1022, 944/1022, 945/1022, 946/1022, 947/1022, 948/1022, 949/1022, 950/1022, 951/1022, 952/1022, 953/1022, 954/1022, 955/1022, 956/1022, 957/1022, 958/1022, 959/1022, 960/1022, 961/1022, 962/1022, 963/1022, 964/1022, 965/1022, 966/1022, 967/1022, 968/1022, 969/1022, 970/1022, 971/1022, 972/1022, 973/1022, 974/1022, 975/1022, 976/1022, 977/1022, 978/1022, 979/1022, 980/1022, 981/1022, 982/1022, 983/1022, 984/1022, 985/1022, 986/1022, 987/1022, 988/1022, 989/1022, 990/1022, 991/1022, 992/1022, 993/1022, 994/1022, 995/1022, 996/1022, 997/1022, 998/1022, 999/1022, 1000/1022, 1001/1022, 1002/1022, 1003/1022, 1004/1022, 1005/1022, 1006/1022, 1007/1022, 1008/1022, 1009/1022, 1010/1022, 1011/1022, 1012/1022, 1013/1022, 1014/1022, 1015/1022, 1016/1022, 1017/1022, 1018/1022, 1019/1022, and 1020/1022.

106. The isolated antibody of claim 95, wherein the antibody comprises a V_{H}/V_{L} pair, or a variant thereof, selected from the group consisting of SEQ ID NOS: 854/1023, 855/1023, 856/1023, 857/1023, 858/1023, 859/1023, 860/1023, 861/1023, 862/1023, 863/1023, 864/1023, 865/1023, 866/1023, 867/1023, 868/1023, 869/1023, 870/1023, 871/1023, 872/1023, 873/1023, 874/1023, 875/1023, 876/1023, 877/1023, 878/1023, 879/1023, 880/1023, 881/1023, 882/1023, 883/1023, 884/1023, 885/1023, 886/1023, 887/1023, 888/1023, 889/1023, 890/1023, 891/1023, 892/1023, 893/1023, 894/1023, 895/1023, 896/1023, 897/1023, 898/1023, 899/1023, 900/1023, 901/1023, 902/1023, 903/1023, 904/1023, 905/1023, 906/1023, 907/1023, 908/1023, 909/1023, 910/1023, 911/1023, 912/1023, 913/1023, 914/1023, 915/1023, 916/1023, 917/1023, 918/1023, 919/1023, 920/1023, 921/1023, 922/1023, 923/1023, 924/1023, 925/1023, 926/1023, 927/1023, 928/1023, 929/1023, 930/1023, 931/1023, 932/1023, 933/1023, 934/1023, 935/1023, 936/1023, 937/1023, 938/1023, 939/1023, 940/1023, 941/1023, 942/1023, 943/1023, 944/1023, 945/1023, 946/1023, 947/1023, 948/1023, 949/1023, 950/1023, 951/1023, 952/1023, 953/1023, 954/1023, 955/1023, 956/1023, 957/1023, 958/1023, 959/1023, 960/1023, 961/1023, 962/1023, 963/1023, 964/1023, 965/1023, 966/1023, 967/1023, 968/1023, 969/1023, 970/1023, 971/1023, 972/1023, 973/1023, 974/1023, 975/1023, 976/1023, 977/1023, 978/1023, 979/1023, 980/1023, 981/1023, 982/1023, 983/1023, 984/1023, 985/1023, 986/1023, 987/1023, 988/1023, 989/1023, 990/1023, 991/1023, 992/1023, 993/1023, 994/1023, 995/1023, 996/1023, 997/1023, 998/1023, 999/1023, 1000/1023, 1001/1023, 1002/1023, 1003/1023, 1004/1023, 1005/1023, 1006/1023, 1007/1023, 1008/1023, 1009/1023, 1010/1023, 1011/1023, 1012/1023, 1013/1023, 1014/1023, 1015/1023, 1016/1023, 1017/1023, 1018/1023, 1019/1023, and 1020/1023.

107. The isolated antibody of claim 95, wherein the antibody comprises a V_{H}/V_{L} pair, or a variant thereof, selected from the group consisting of SEQ ID NOS: 854/1024, 855/1024, 856/1024, 857/1024, 858/1024, 859/1024, 860/1024, 861/1024, 862/1024, 863/1024, 864/1024, 865/1024, 866/1024, 867/1024, 868/1024, 869/1024, 870/1024, 871/1024, 872/1024, 873/1024, 874/1024, 875/1024, 876/1024, 877/1024, 878/1024, 879/1024, 880/1024, 881/1024, 882/1024, 883/1024, 884/1024, 885/1024, 886/1024, 887/1024, 888/1024, 889/1024, 890/1024, 891/1024, 892/1024, 893/1024, 894/1024, 895/1024, 896/1024, 897/1024, 898/1024, 899/1024, 900/1024, 901/1024, 902/1024, 903/1024, 904/1024, 905/1024, 906/1024, 907/1024, 908/1024, 909/1024, 910/1024, 911/1024, 912/1024, 913/1024, 914/1024, 915/1024, 916/1024, 917/1024, 918/1024, 919/1024, 920/1024, 921/1024, 922/1024, 923/1024, 924/1024, 925/1024, 926/1024, 927/1024, 928/1024, 929/1024, 930/1024, 931/1024, 932/1024, 933/1024, 934/1024, 935/1024, 936/1024, 937/1024, 938/1024, 939/1024, 940/1024, 941/1024, 942/1024, 943/1024, 944/1024, 945/1024, 946/1024, 947/1024, 948/1024, 949/1024, 950/1024, 951/1024, 952/1024, 953/1024, 954/1024, 955/1024, 956/1024, 957/1024, 958/1024, 959/1024, 960/1024, 961/1024, 962/1024, 963/1024, 964/1024, 965/1024, 966/1024, 967/1024, 968/1024, 969/1024, 970/1024, 971/1024, 972/1024, 973/1024, 974/1024, 975/1024, 976/1024, 977/1024, 978/1024, 979/1024, 980/1024, 981/1024, 982/1024, 983/1024, 984/1024, 985/1024, 986/1024, 987/1024, 988/1024, 989/1024, 990/1024, 991/1024, 992/1024, 993/1024, 994/1024, 995/1024, 996/1024, 997/1024, 998/1024, 999/1024, 1000/1024, 1001/1024, 1002/1024, 1003/1024, 1004/1024, 1005/1024, 1006/1024, 1007/1024, 1008/1024, 1009/1024, 1010/1024, 1011/1024, 1012/1024, 1013/1024, 1014/1024, 1015/1024, 1016/1024, 1017/1024, 1018/1024, 1019/1024, and 1020/1024.

108. The isolated antibody of claim 95, wherein the antibody comprises a V_{H}/V_{L} pair, or a variant thereof, selected from the group consisting of SEQ ID NOS: 854/1025, 855/1025, 856/1025, 857/1025, 858/1025, 859/1025, 860/1025, 861/1025, 862/1025, 863/1025, 864/1025, 865/1025, 866/1025, 867/1025, 868/1025, 869/1025, 870/1025, 871/1025, 872/1025, 873/1025, 874/1025, 875/1025, 876/1025, 877/1025, 878/1025, 879/1025, 880/1025, 881/1025, 882/1025, 883/1025, 884/1025, 885/1025, 886/1025, 887/1025, 888/1025, 889/1025, 890/1025, 891/1025, 892/1025, 893/1025, 894/1025, 895/1025, 896/1025, 897/1025, 898/1025, 899/1025, 900/1025, 901/1025, 902/1025, 903/1025, 904/1025, 905/1025, 906/1025, 907/1025, 908/1025, 909/1025, 910/1025, 911/1025, 912/1025, 913/1025, 914/1025, 915/1025, 916/1025, 917/1025, 918/1025, 919/1025, 920/1025, 921/1025, 922/1025, 923/1025, 924/1025, 925/1025, 926/1025, 927/1025, 928/1025, 929/1025, 930/1025, 931/1025, 932/1025, 933/1025, 934/1025, 935/1025, 936/1025, 937/1025, 938/1025, 939/1025, 940/1025, 941/1025, 942/1025, 943/1025, 944/1025, 945/1025, 946/1025, 947/1025, 948/1025, 949/1025, 950/1025, 951/1025, 952/1025, 953/1025, 954/1025, 955/1025, 956/1025, 957/1025, 958/1025, 959/1025, 960/1025, 961/1025, 962/1025, 963/1025, 964/1025, 965/1025, 966/1025, 967/1025, 968/1025, 969/1025, 970/1025, 971/1025, 972/1025, 973/1025, 974/1025, 975/1025, 976/1025, 977/1025, 978/1025, 979/1025, 980/1025, 981/1025, 982/1025, 983/1025, 984/1025, 985/1025, 986/1025, 987/1025, 988/1025, 989/1025, 990/1025, 991/1025, 992/1025, 993/1025, 994/1025, 995/1025, 996/1025, 997/1025, 998/1025, 999/1025, 1000/1025, 1001/1025, 1002/1025, 1003/1025, 1004/1025, 1005/1025, 1006/1025, 1007/1025, 1008/1025, 1009/1025, 1010/1025, 1011/1025, 1012/1025, 1013/1025, 1014/1025, 1015/1025, 1016/1025, 1017/1025, 1018/1025, 1019/1025, and 1020/1025.

109. The isolated antibody of claim 95, wherein the antibody comprises a V_{H}/V_{L} pair, or a variant thereof, selected from the group consisting of SEQ ID NOS: 854/1026, 855/1026, 856/1026, 857/1026, 858/1026, 859/1026, 860/1026, 861/1026, 862/1026, 863/1026, 864/1026, 865/1026, 866/1026, 867/1026, 868/1026, 869/1026, 870/1026, 871/1026, 872/1026, 873/1026, 874/1026, 875/1026, 876/1026, 877/1026, 878/1026, 879/1026, 880/1026, 881/1026, 882/1026, 883/1026, 884/1026, 885/1026, 886/1026, 887/1026, 888/1026, 889/1026, 890/1026, 891/1026, 892/1026, 893/1026, 894/1026, 895/1026, 896/1026, 897/1026, 898/1026, 899/1026, 900/1026, 901/1026, 902/1026, 903/1026, 904/1026, 905/1026, 906/1026, 907/1026, 908/1026, 909/1026, 910/1026, 911/1026, 912/1026, 913/1026, 914/1026, 915/1026, 916/1026, 917/1026, 918/1026, 919/1026, 920/1026, 921/1026, 922/1026, 923/1026, 924/1026, 925/1026, 926/1026, 927/1026, 928/1026, 929/1026, 930/1026, 931/1026, 932/1026, 933/1026, 934/1026, 935/1026, 936/1026, 937/1026, 938/1026, 939/1026, 940/1026, 941/1026, 942/1026, 943/1026, 944/1026, 945/1026, 946/1026, 947/1026, 948/1026, 949/1026, 950/1026, 951/1026, 952/1026, 953/1026, 954/1026, 955/1026, 956/1026, 957/1026, 958/1026, 959/1026, 960/1026, 961/1026, 962/1026, 963/1026, 964/1026, 965/1026, 966/1026, 967/1026, 968/1026, 969/1026, 970/1026, 971/1026, 972/1026, 973/1026, 974/1026, 975/1026, 976/1026, 977/1026, 978/1026, 979/1026, 980/1026, 981/1026, 982/1026, 983/1026, 984/1026, 985/1026, 986/1026, 987/1026, 988/1026, 989/1026, 990/1026, 991/1026, 992/1026, 993/1026, 994/1026, 995/1026, 996/1026, 997/1026, 998/1026, 999/1026, 1000/1026, 1001/1026, 1002/1026, 1003/1026, 1004/1026, 1005/1026, 1006/1026, 1007/1026, 1008/1026, 1009/1026, 1010/1026, 1011/1026, 1012/1026, 1013/1026, 1014/1026, 1015/1026, 1016/1026, 1017/1026, 1018/1026, 1019/1026, and 1020/1026.

110. The isolated antibody of claim 95, wherein the antibody comprises three Chothia HC CDRs according to one of the following groups of HC CDR SEQ ID NOS: 4/338/672; 5/339/673; 6/340/674; 7/341/675; 8/342/676; 9/343/677; 10/344/678; 11/345/679; 12/346/680; 13/347/681; 14/348/682; 15/349/683; 16/350/684; 17/351/685; 18/352/686; 19/353/687; 20/354/688; 21/355/689; 22/356/690; 23/357/691; 24/358/692; 25/359/693; 26/360/694; 27/361/695; 28/362/696; 29/363/697; 30/364/698; 31/365/699; 32/366/700; 33/367/701; 34/368/702; 35/369/703; 36/370/704; 37/371/705; 38/372/706; 39/373/707; 40/374/708; 41/375/709; 42/376/710; 43/377/711; 44/378/712; 45/379/713; 46/380/714; 47/381/715; 48/382/716; 49/383/717; 50/384/718; 51/385/719; 52/386/720; 53/387/721; 54/388/722; 55/389/723; 56/390/724; 57/391/725; 58/392/726; 59/393/727; 60/394/728; 61/395/729; 62/396/730; 63/397/731; 64/398/732; 65/399/733; 66/400/734; 67/401/735; 68/402/736; 69/403/737; 70/404/738; 71/405/739; 72/406/740; 73/407/741; 74/408/742; 75/409/743; 76/410/744; 77/411/745; 78/412/746; 79/413/747; 80/414/748; 81/415/749; 82/416/750; 83/417/751; 84/418/752; 85/419/753; 86/420/754; 87/421/755; 88/422/756; 89/423/757; 90/424/758; 91/425/759; 92/426/760; 93/427/761; 94/428/762; 95/429/763; 96/430/764; 97/431/765; 98/432/766; 99/433/767; 100/434/768; 101/435/769; 102/436/770; 103/437/771; 104/438/772; 105/439/773; 106/440/774; 107/441/775; 108/442/776; 109/443/777; 110/444/778; 111/445/779; 112/446/780; 113/447/781; 114/448/782; 115/449/783; 116/450/784; 117/451/785; 118/452/786; 119/453/787; 120/454/788; 121/455/789; 122/456/790; 123/457/791; 124/458/792; 125/459/793; 126/460/794; 127/461/795; 128/462/796; 129/463/797; 130/464/798; 131/465/799; 132/466/800; 133/467/801; 134/468/802; 135/469/803; 136/470/804; 137/471/805; 138/472/806; 139/473/807; 140/474/808; 141/475/809; 142/476/810; 143/477/811; 144/478/812; 145/479/813; 146/480/814; 147/481/815; 148/482/816; 149/483/817; 150/484/818; 151/485/819; 152/486/820; 153/487/821; 154/488/822; 155/489/823; 156/490/824; 157/491/825; 158/492/826; 159/493/827; 160/494/828; 161/495/829; 162/496/830; 163/497/831; 164/498/832; 165/499/833; 166/500/834; 167/501/835; 168/502/836; 169/503/837; and 170/504/838.

111. The isolated antibody of claim 95, wherein the antibody comprises six CDRs according to one of the following groups of CDR SEQ ID NOs: 4/338/672/839/844/849; 5/339/673/839/844/849; 6/340/674/839/844/849; 7/341/675/839/844/849; 8/342/676/839/844/849; 9/343/677/839/844/849; 10/344/678/839/844/849; 11/345/679/839/844/849; 12/346/680/839/844/849; 13/347/681/839/844/849; 14/348/682/839/844/849; 15/349/683/839/844/849; 16/350/684/839/844/849; 17/351/685/839/844/849; 18/352/686/839/844/849; 19/353/687/839/844/849; 20/354/688/839/844/849; 21/355/689/839/844/849; 22/356/690/839/844/849; 23/357/691/839/844/849; 24/358/692/839/844/849; 25/359/693/839/844/849; 26/360/694/839/844/849; 27/361/695/839/844/849; 28/362/696/839/844/849; 29/363/697/839/844/849; 30/364/698/839/844/849; 31/365/699/839/844/849; 32/366/700/839/844/849; 33/367/701/839/844/849; 34/368/702/839/844/849; 35/369/703/839/844/849; 36/370/704/839/844/849; 37/371/705/839/844/849; 38/372/706/839/844/849; 39/373/707/839/844/849; 40/374/708/839/844/849; 41/375/709/839/844/849; 42/376/710/839/844/849; 43/377/711/839/844/849; 44/378/712/839/844/849; 45/379/713/839/844/849; 46/380/714/839/844/849; 47/381/715/839/844/849; 48/382/716/839/844/849; 49/383/717/839/844/849; 50/384/718/839/844/849; 51/385/719/839/844/849; 52/386/720/839/844/849; 53/387/721/839/844/849; 54/388/722/839/844/849; 55/389/723/839/844/849; 56/390/724/839/844/849; 57/391/725/839/844/849; 58/392/726/839/844/849; 59/393/727/839/844/849; 60/394/728/839/844/849; 61/395/729/839/844/849; 62/396/730/839/844/849; 63/397/731/839/844/849; 64/398/732/839/844/849; 65/399/733/839/844/849; 66/400/734/839/844/849; 67/401/735/839/844/849; 68/402/736/839/844/849; 69/403/737/839/844/849; 70/404/738/839/844/849; 71/405/739/839/844/849; 72/406/740/839/844/849; 73/407/741/839/844/849; 74/408/742/839/844/849; 75/409/743/839/844/849; 76/410/744/839/844/849; 77/411/745/839/844/849; 78/412/746/839/844/849; 79/413/747/839/844/849; 80/414/748/839/844/849; 81/415/749/839/844/849; 82/416/750/839/844/849; 83/417/751/839/844/849; 84/418/752/839/844/849; 85/419/753/839/844/849; 86/420/754/839/844/849; 87/421/755/839/844/849; 88/422/756/839/844/849; 89/423/757/839/844/849; 90/424/758/839/844/849; 91/425/759/839/844/849; 92/426/760/839/844/849; 93/427/761/839/844/849; 94/428/762/839/844/849; 95/429/763/839/844/849; 96/430/764/839/844/849; 97/431/765/839/844/849; 98/432/766/839/844/849; 99/433/767/839/844/849; 100/434/768/839/844/849; 101/435/769/839/844/849; 102/436/770/839/844/849; 103/437/771/839/844/849; 104/438/772/839/844/849; 105/439/773/839/844/849; 106/440/774/839/844/849; 107/441/775/839/844/849; 108/442/776/839/844/849; 109/443/777/839/844/849; 110/444/778/839/844/849; 111/445/779/839/844/849; 112/446/780/839/844/849; 113/447/781/839/844/849; 114/448/782/839/844/849; 115/449/783/839/844/849; 116/450/784/839/844/849; 117/451/785/839/844/849; 118/452/786/839/844/849; 119/453/787/839/844/849; 120/454/788/839/844/849; 121/455/789/839/844/849; 122/456/790/839/844/849; 123/457/791/839/844/849; 124/458/792/839/844/849; 125/459/793/839/844/849; 126/460/794/839/844/849; 127/461/795/839/844/849; 128/462/796/839/844/849; 129/463/797/839/844/849; 130/464/798/839/844/849; 131/465/799/839/844/849; 132/466/800/839/844/849; 133/467/801/839/844/849; 134/468/802/839/844/849; 135/469/803/839/844/849; 136/470/804/839/844/849; 137/471/805/839/844/849; 138/472/806/839/844/849; 139/473/807/839/844/849; 140/474/808/839/844/849; 141/475/809/839/844/849; 142/476/810/839/844/849; 143/477/811/839/844/849; 144/478/812/839/844/849; 145/479/813/840/845/850; 146/480/814/840/845/850; 147/481/815/840/845/850; 148/482/816/840/845/850; 149/483/817/840/845/850; 150/484/818/840/845/850; 151/485/819/840/845/850; 152/486/820/840/845/850; 153/487/821/840/845/850; 154/488/822/840/845/850; 155/489/823/840/845/850; 156/490/824/840/845/850; 157/491/825/840/845/850; 158/492/826/840/845/850; 159/493/827/840/845/850; 160/494/828/840/845/850; 161/495/829/840/845/850; 162/496/830/840/845/850; 163/497/831/840/845/850; 164/498/832/840/845/850; 165/499/833/840/845/850; 166/500/834/840/845/850; 167/501/835/840/845/850; 168/502/836/840/845/850; 169/503/837/840/845/850; 170/504/838/840/845/850; 145/479/813/841/846/851; 146/480/814/841/846/851; 147/481/815/841/846/851; 148/482/816/841/846/851; 149/483/817/841/846/851; 150/484/818/841/846/851; 151/485/819/841/846/851; 152/486/820/841/846/851; 153/487/821/841/846/851; 154/488/822/841/846/851; 155/489/823/841/846/851; 156/490/824/841/846/851; 157/491/825/841/846/851; 158/492/826/841/846/851; 159/493/827/841/846/851; 160/494/828/841/846/851; 161/495/829/841/846/851; 162/496/830/841/846/851; 163/497/831/841/846/851; 164/498/832/841/846/851; 165/499/833/841/846/851; 166/500/834/841/846/851; 167/501/835/841/846/851; 168/502/836/841/846/851; 169/503/837/841/846/851; and 170/504/838/841/846/851.

112. The isolated antibody of claim 95, wherein the antibody comprises three Kabat HC CDRs according to one of the following groups of HC CDR SEQ ID NOS: 171/505/672; 172/506/673; 173/507/674; 174/508/675; 175/509/676; 176/510/677; 177/511/678; 178/512/679; 179/513/680; 180/514/681; 181/515/682; 182/516/683; 183/517/684; 184/518/685; 185/519/686; 186/520/687; 187/521/688; 188/522/689; 189/523/690; 190/524/691; 191/525/692; 192/526/693; 193/527/694; 194/528/695; 195/529/696; 196/530/697; 197/531/698; 198/532/699; 199/533/700; 200/534/701; 201/535/702; 202/536/703; 203/537/704; 204/538/705; 205/539/706; 206/540/707; 207/541/708; 208/542/709; 209/543/710; 210/544/711; 211/545/712; 212/546/713; 213/547/714; 214/548/715; 215/549/716; 216/550/717; 217/551/718; 218/552/719; 219/553/720; 220/554/721; 221/555/722; 222/556/723; 223/557/724; 224/558/725; 225/559/726; 226/560/727; 227/561/728; 228/562/729; 229/563/730; 230/564/731; 231/565/732; 232/566/733; 233/567/734; 234/568/735; 235/569/736; 236/570/737; 237/571/738; 238/572/739; 239/573/740; 240/574/741; 241/575/742; 242/576/743; 243/577/744; 244/578/745; 245/579/746; 246/580/747; 247/581/748; 248/582/749; 249/583/750; 250/584/751; 251/585/752; 252/586/753; 253/587/754; 254/588/755; 255/589/756; 256/590/757; 257/591/758; 258/592/759; 259/593/760; 260/594/761; 261/595/762; 262/596/763; 263/597/764; 264/598/765; 265/599/766; 266/600/767; 267/601/768; 268/602/769; 269/603/770; 270/604/771; 271/605/772; 272/606/773; 273/607/774; 274/608/775; 275/609/776; 276/610/777; 277/611/778; 278/612/779; 279/613/780; 280/614/781; 281/615/782; 282/616/783; 283/617/784; 284/618/785; 285/619/786; 286/620/787; 287/621/788; 288/622/789; 289/623/790; 290/624/791; 291/625/792; 292/626/793; 293/627/794; 294/628/795; 295/629/796; 296/630/797; 297/631/798; 298/632/799; 299/633/800; 300/634/801; 301/635/802; 302/636/803; 303/637/804; 304/638/805; 305/639/806; 306/640/807; 307/641/808; 308/642/809; 309/643/810; 310/644/811; 311/645/812; 312/646/813; 313/647/814; 314/648/815; 315/649/816; 316/650/817; 317/651/818; 318/652/819; 319/653/820; 320/654/821; 321/655/822; 322/656/823; 323/657/824; 324/658/825; 325/659/826; 326/660/827; 327/661/828; 328/662/829; 329/663/830; 330/664/831; 331/665/832; 332/666/833; 333/667/834; 334/668/835; 335/669/836; 336/670/837; and 337/671/838.

113. The isolated antibody of claim 95, wherein the antibody comprises six CDRs according to one of the following groups of CDR SEQ ID NOS: 171/505/672/839/844/849; 172/506/673/839/844/849; 173/507/674/839/844/849; 174/508/675/839/844/849; 175/509/676/839/844/849; 176/510/677/839/844/849; 177/511/678/839/844/849; 178/512/679/839/844/849; 179/513/680/839/844/849; 180/514/681/839/844/849; 181/515/682/839/844/849; 182/516/683/839/844/849; 183/517/684/839/844/849; 184/518/685/839/844/849; 185/519/686/839/844/849; 186/520/687/839/844/849; 187/521/688/839/844/849; 188/522/689/839/844/849; 189/523/690/839/844/849; 190/524/691/839/844/849; 191/525/692/839/844/849; 192/526/693/839/844/849; 193/527/694/839/844/849; 194/528/695/839/844/849; 195/529/696/839/844/849; 196/530/697/839/844/849; 197/531/698/839/844/849; 198/532/699/839/844/849; 199/533/700/839/844/849; 200/534/701/839/844/849; 201/535/702/839/844/849; 202/536/703/839/844/849; 203/537/704/839/844/849; 204/538/705/839/844/849; 205/539/706/839/844/849; 206/540/707/839/844/849; 207/541/708/839/844/849; 208/542/709/839/844/849; 209/543/710/839/844/849; 210/544/711/839/844/849; 211/545/712/839/844/849; 212/546/713/839/844/849; 213/547/714/839/844/849; 214/548/715/839/844/849; 215/549/716/839/844/849; 216/550/717/839/844/849; 217/551/718/839/844/849; 218/552/719/839/844/849; 219/553/720/839/844/849; 220/554/721/839/844/849; 221/555/722/839/844/849; 222/556/723/839/844/849; 223/557/724/839/844/849; 224/558/725/839/844/849; 225/559/726/839/844/849; 226/560/727/839/844/849; 227/561/728/839/844/849; 228/562/729/839/844/849; 229/563/730/839/844/849; 230/564/731/839/844/849; 231/565/732/839/844/849; 232/566/733/839/844/849; 233/567/734/839/844/849; 234/568/735/839/844/849; 235/569/736/839/844/849; 236/570/737/839/844/849; 237/571/738/839/844/849; 238/572/739/839/844/849; 239/573/740/839/844/849; 240/574/741/839/844/849; 241/575/742/839/844/849; 242/576/743/839/844/849; 243/577/744/839/844/849; 244/578/745/839/844/849; 245/579/746/839/844/849; 246/580/747/839/844/849; 247/581/748/839/844/849; 248/582/749/839/844/849; 249/583/750/839/844/849; 250/584/751/839/844/849; 251/585/752/839/844/849; 252/586/753/839/844/849; 253/587/754/839/844/849; 254/588/755/839/844/849; 255/589/756/839/844/849; 256/590/757/839/844/849; 257/591/758/839/844/849; 258/592/759/839/844/849; 259/593/760/839/844/849; 260/594/761/839/844/849; 261/595/762/839/844/849; 262/596/763/839/844/849; 263/597/764/839/844/849; 264/598/765/839/844/849; 265/599/766/839/844/849; 266/600/767/839/844/849; 267/601/768/839/844/849; 268/602/769/839/844/849; 269/603/770/839/844/849; 270/604/771/839/844/849; 271/605/772/839/844/849; 272/606/773/839/844/849; 273/607/774/839/844/849; 274/608/775/839/844/849; 275/609/776/839/844/849; 276/610/777/839/844/849; 277/611/778/839/844/849; 278/612/779/839/844/849; 279/613/780/839/844/849; 280/614/781/839/844/849; 281/615/782/839/844/849; 282/616/783/839/844/849; 283/617/784/839/844/849; 284/618/785/839/844/849; 285/619/786/839/844/849; 286/620/787/839/844/849; 287/621/788/839/844/849; 288/622/789/839/844/849; 289/623/790/839/844/849; 290/624/791/839/844/849; 291/625/792/839/844/849; 292/626/793/839/844/849; 293/627/794/839/844/849; 294/628/795/839/844/849; 295/629/796/839/844/849; 296/630/797/839/844/849; 297/631/798/839/844/849; 298/632/799/839/844/849; 299/633/800/839/844/849; 300/634/801/839/844/849; 301/635/802/839/844/849; 302/636/803/839/844/849; 303/637/804/839/844/849; 304/638/805/839/844/849; 305/639/806/839/844/849; 306/640/807/839/844/849; 307/641/808/839/844/849; 308/642/809/839/844/849; 309/643/810/839/844/849; 310/644/811/839/844/849; 311/645/812/840/845/850; 312/646/813/840/845/850; 313/647/814/840/845/850; 314/648/815/840/845/850; 315/649/816/840/845/850; 316/650/817/840/845/850; 317/651/818/840/845/850; 318/652/819/840/845/850; 319/653/820/840/845/850; 320/654/821/840/845/850; 321/655/822/840/845/850; 322/656/823/840/845/850; 323/657/824/840/845/850; 324/658/825/840/845/850; 325/659/826/840/845/850; 326/660/827/840/845/850; 327/661/828/840/845/850; 328/662/829/840/845/850; 329/663/830/840/845/850; 330/664/831/840/845/850; 331/665/832/840/845/850; 332/666/833/840/845/850; 333/667/834/840/845/850; 334/668/835/840/845/850; 335/669/836/840/845/850; 336/670/837/840/845/850; 337/671/838/840/845/850; 311/645/812/841/846/851; 312/646/813/841/846/851; 313/647/814/841/846/851; 314/648/815/841/846/851; 315/649/816/841/846/851; 316/650/817/841/846/851; 317/651/818/841/846/851; 318/652/819/841/846/851; 319/653/820/841/846/851; 320/654/821/841/846/851; 321/655/822/841/846/851; 322/656/823/841/846/851; 323/657/824/841/846/851; 324/658/825/841/846/851; 325/659/826/841/846/851; 326/660/827/841/846/851; 327/661/828/841/846/851; 328/662/829/841/846/851; 329/663/830/841/846/851; 330/664/831/841/846/851; 331/665/832/841/846/851; 332/666/833/841/846/851; 333/667/834/841/846/851; 334/668/835/841/846/851; 335/669/836/841/846/851; 336/670/837/841/846/851; and 337/671/838/841/846/851.

114. The isolated antibody of any of claims 95-109, wherein the antibody comprises:
(a) three heavy chain CDRs and three light chain CDRs of the V_{H} region SEQ ID NO: 854, and the V_{L} region SEQ ID NO: 1021;
(b) three heavy chain CDRs and three light chain CDRs of the V_{H} region SEQ ID NO: 859, and the V_{L} region SEQ ID NO: 1021; or
(c) three heavy chain CDRs and three light chain CDRs of the V_{H} region SEQ ID NO: 895, and the V_{L} region SEQ ID NO: 1021.

115. The isolated antibody of claim 114, wherein the antibody comprises three heavy chain CDRs and three light chain CDRs of the V_{H} region SEQ ID NO: 854, and the V_{L} region SEQ ID NO: 1021.

116. The isolated antibody of any of claims 95-115, wherein the antibody comprises:
(a) a V_{H} comprising: a CDR-H1 comprising at least one of SEQ ID NOs: 4 and 171; a CDR-H2 comprising at least one of SEQ ID NOs: 338 and 505; and a CDR-H3 comprising SEQ ID NO: 672; and a V_{L} comprising: a CDR-L1 comprising SEQ ID NO: 839; a CDR-L2 comprising SEQ ID NO: 844; and a CDR-L3 comprising SEQ ID NO: 849;
(b) a V_{H} comprising: a CDR-H1 comprising at least one of SEQ ID NOs: 9 and 176; a CDR-H2 comprising at least one of SEQ ID NOs: 343 and 510; and a CDR-H3 comprising SEQ ID NO: 677; and a V_{L} comprising: a CDR-L1 comprising SEQ ID NO: 839; a CDR-L2 comprising SEQ ID NO: 844; and a CDR-L3 comprising SEQ ID NO: 849; or
(c) a V_{H} comprising: a CDR-H1 comprising at least one of SEQ ID NOs: 45 and 212; a CDR-H2 comprising at least one of SEQ ID NOs: 379 and 546; and a CDR-H3 comprising SEQ ID NO: 713; and a V_{L} comprising: a CDR-L1 comprising SEQ ID NO: 839; a CDR-L2 comprising SEQ ID NO: 844; and a CDR-L3 comprising SEQ ID NO: 849.

117. The isolated antibody of any of claims 95-116, wherein the antibody comprises a V_{H} comprising: a CDR-H1 comprising at least one of SEQ ID NOs: 4 and 171; a CDR-H2 comprising at least one of SEQ ID NOs: 338 and 505; and a CDR-H3 comprising SEQ ID NO: 672; and a V_{L} comprising: a CDR-L1 comprising SEQ ID NO: 839; a CDR-L2 comprising SEQ ID NO: 844; and a CDR-L3 comprising SEQ ID NO: 849.

118. The isolated antibody of any of claims 95-117, wherein the variant thereof has 20, 15, 10, 9, 8, 7, 6, 5, 4, 3, 2, or 1 amino acid substitutions.

119. The isolated antibody of claim 118, wherein the amino acid substitutions are conservative amino acid substitutions.

120. The isolated antibody of any of claims 95-117, wherein the variant thereof has 90%, 95%, 96%, 97%, 98%, or 99% sequence identity.

121. An isolated antibody that specifically binds to ROR1, wherein the antibody binds to the same epitope as a second antibody with a V_{H} region selected from SEQ ID NOs: 854-1020 and a V_{L} region selected from SEQ ID NOs: 1021-1026.

122. The isolated antibody of claim 121, wherein the isolated antibody inhibits binding of the second antibody to ROR1 by at least 50%, or wherein the second antibody inhibits binding of the isolated antibody to ROR1 by at least 50%.

123. The isolated antibody of any one of claims 95-122, wherein the antibody has a kₐ of about 9.17×10⁴ M⁻¹×sec⁻¹ to about 5.94×10⁶ M⁻¹×sec⁻¹ when associating with human ROR1 at a temperature of 25° C.

124. The isolated antibody of any one of claims 95-123, wherein the antibody has a kₐ of about 1.01×10⁴ sec⁻¹ to about 0.05 sec⁻¹ when dissociating from human ROR1 at a temperature of 25° C.

125. The isolated antibody of any one of claims 95-124, wherein the antibody has a K_{D} of about 1.39×10⁻¹⁰ M to about 1.01×10⁻⁷ M when bound to human ROR1 at a temperature of 25° C.

126. The isolated antibody of any one of claims 95-125, wherein the antibody comprises at least one constant region domain.

127. The isolated antibody of claim 126, wherein the constant region comprises a sequence selected from SEQ ID Nos: 1027-1029.

128. The isolated antibody of any one of claims 95-127, wherein the antibody is a monoclonal antibody.

129. The isolated antibody of any one of claims 95-128, wherein the antibody is an IgA, an IgD, an IgE, an IgG, or an IgM.

130. The isolated antibody of claims 95-129, wherein the antibody is humanized or human.

131. The isolated antibody of any one of claims 95-130, wherein the antibody is aglycosylated.

132. The isolated antibody of claims 95-131, wherein the antibody is an antibody fragment.

133. The isolated antibody of claim 132, wherein the antibody fragment is selected from an Fv fragment, a Fab fragment, a F(ab′)₂ fragment, a Fab′ fragment, an scFv (sFv) fragment, and an scFv-Fc fragment.

134. The isolated antibody of claim 133, wherein the antibody is an scFv fragment.

135. A kit comprising the antibody conjugate of any one of claims 1-94 or the isolated antibody of any one of claims 95-134, and instructions for use.

136. The kit of claim 135, wherein the antibody conjugate or isolated antibody is lyophilized.

137. The kit of claim 136, further comprising a fluid for reconstitution of the lyophilized antibody conjugate or isolated antibody.

138. A polynucleotide encoding an antibody of any of claims 95-134.

139. A vector comprising the polynucleotide of claim 138.

140. A recombinant host cell comprising the vector of claim 139.

141. The host cell of claim 140, wherein the host cell is selected from a bacterial cell, a fungal cell, and a mammalian cell.

142. The host cell of claim 141, wherein the host cell is selected from an E. coli cell, a Saccharomyces cerevisiae cell, and a CHO cell.

143. A cell-free expression reaction comprising the vector of claim 139.

144. A pharmaceutical composition comprising the antibody conjugate of any of claims 1 to 94 or the isolated antibody of any one of claims 95-134 and a pharmaceutically acceptable carrier.

145. A pharmaceutical composition comprising (a) the antibody conjugate of any of claims 1 to 94 or the isolated antibody of any one of claims 95-134, (b) one or more additional active agents; and (c) a pharmaceutically acceptable carrier.

146. The pharmaceutical composition of claim 145, wherein the one or more additional active agents is one or more checkpoint inhibitors.

147. The pharmaceutical composition of claim 146, wherein the one or more checkpoint inhibitors are a PD-1 inhibitor, PD-L1 inhibitor, PD-L2 inhibitor, CTLA-4 inhibitor, LAG-3 inhibitor, or TIM-3 inhibitor.

148. The pharmaceutical composition of claim 147, wherein the one or more checkpoint inhibitors are a PD-1 inhibitor or PD-L1 inhibitor.

149. The pharmaceutical composition of claim 148, wherein the one or more PD-1 or PD-L1 inhibitors are a small molecule blocker of the PD-1 or PD-L1 pathway.

150. The pharmaceutical composition of claim 148, wherein the one or more PD-1 or PD-L1 inhibitors are an antibody that inhibits PD-1 or PD-L1 activity.

151. The pharmaceutical composition of any one of claims 147 to 150, wherein the one or more PD-1 or PD L1 inhibitors are selected from the group consisting of CA-170, BMS 8, BMS-202, BMS-936558, CK-301, AUNP12, avelumab, nivolumab, pembrolizumab, atezolizumab, durvalumab, AMP 224, MEDI0680/AMP-514, PDR001, cemiplimab, TSR-042, Tizlelizumab/BGB-A317, CK 301, BMS-936559, camrelizumab, sintilimab, toripalimab, genolimzumab, A167, MGA012, PF-06801591, LY3300054, FAZ053, PD-11, CX-072, BGB-A333, BI 754091, JNJ 63723283, AGEN2034, CA-327, CX-188, STI-A1110, JTX-4014, (LLY) AM0001, CBT-502, FS118, XmAb20717, XmAb23104, AB122, KY1003, RXI-762, PRS-33, ALPN-202, TSR-075, MCLA 145, MGD013, and MGD019.

152. The pharmaceutical composition of claim 145, wherein the one or more additional active agents is one or more PARP inhibitors.

153. The pharmaceutical composition of claim 152, wherein the one or more PARP inhibitors are selected from olaparib, rucaparib, niraparib, and talazoparib.

154. The pharmaceutical composition of claim 153, wherein the one or more PARP inhibitor is olaparib.

155. A method of reducing cell proliferation in a subject in need thereof, comprising administering to the subject an effective amount of an antibody conjugate of any of claims 1 to 94 or the isolated antibody of any one of claims 95-134, or a pharmaceutical composition of any one of claims 144 to 154.

156. A method of treating or preventing a disease or condition in a subject in need thereof, comprising administering to the subject an effective amount of an antibody conjugate of any of claims 1 to 94 or the isolated antibody of any one of claims 95-134, or a pharmaceutical composition of any one of claims 144 to 154.

157. A method of diagnosing a disease or condition in a subject in need thereof, comprising administering to the subject an effective amount of an antibody conjugate of any of claims 1 to 94 or the isolated antibody of any one of claims 95-134, or a pharmaceutical composition of any one of claims 144 to 154.

158. A method of treating or preventing a disease or condition in a subject in need thereof, comprising administering to the subject an effective amount of an antibody conjugate of any of claims 1 to 94 or the isolated antibody of any one of claims 95-134, or a pharmaceutical composition of any one of claims 144 to 154, and administering to the subject one or more additional active agents.

159. The method of claim 158, wherein the effective amount of an antibody conjugate of any of claims 1 to 94 or the isolated antibody of any one of claims 95-134 or a pharmaceutical composition of any one of claims 144 to 154 and the one or more additional active agents are administered sequentially.

160. The method of claim 158, wherein the effective amount of an antibody conjugate of any of claims 1 to 94 or the isolated antibody of any one of claims 95-134 or a pharmaceutical composition of any one of claims 144 to 154 and the one or more additional active agents are administered concurrently.

161. The method of any one of claims 158-160, wherein the one or more additional active agents is one or more checkpoint inhibitors.

162. The method of claim 161, wherein the one or more checkpoint inhibitors are a PD-1 inhibitor, PD-L1 inhibitor, PD-L2 inhibitor, CTLA-4 inhibitor, LAG-3 inhibitor, or TIM-3 inhibitor.

163. The method of claim 162, wherein the one or more checkpoint inhibitors are a PD-1 inhibitor or PD-L1 inhibitor.

164. The method of claim 163, wherein the one or more PD-1 or PD-L1 inhibitors are a small molecule blocker of the PD-1 or PD-L1 pathway.

165. The method of claim 163, wherein the one or more PD-1 or PD-L1 inhibitors are an antibody that inhibits PD-1 or PD-L1 activity.

166. The method of claim 163, wherein the one or more PD-1 or PD L1 inhibitors are selected from the group consisting of CA-170, BMS 8, BMS-202, BMS-936558, CK-301, AUNP12, avelumab, nivolumab, pembrolizumab, atezolizumab, durvalumab, AMP 224, MEDI0680/AMP-514, PDR001, cemiplimab, TSR-042, Tizlelizumab/BGB-A317, CK 301, BMS-936559, camrelizumab, sintilimab, toripalimab, genolimzumab, A167, MGA012, PF-06801591, LY3300054, FAZ053, PD-11, CX-072, BGB-A333, BI 754091, JNJ 63723283, AGEN2034, CA-327, CX-188, STI-A1110, JTX-4014, (LLY) AM0001, CBT-502, FS118, XmAb20717, XmAb23104, AB122, KY1003, RXI-762, PRS-33, ALPN-202, TSR-075, MCLA 145, MGD013, and MGD019.

167. The method of any one of claims 158-160, wherein the one or more additional active agents is one or more PARP inhibitors.

168. The method of claim 167, wherein the one or more PARP inhibitors are selected from olaparib, rucaparib, niraparib, and talazoparib.

169. The method of claim 168, wherein the one or more PARP inhibitor is olaparib.

170. The method of any of any one of claims 155-169, wherein the disease or condition is a cancer.

171. The method of any of claims 155-169, wherein the disease or condition is a hematological cancer.

172. The method of any of claims 155-169, wherein the disease or condition is a solid tumor cancer.

173. The method of any of claims 155-169, wherein the disease or condition is colorectal cancer.

174. The method of any of claims 155-169, wherein the disease or condition is breast cancer.

175. The method of claim 174, wherein the breast cancer is triple negative breast cancer.

176. The method of claim 175, wherein the triple negative breast cancer is PARP inhibitor-resistant.

177. The method of claim 175 or 176, wherein the triple negative breast cancer is BRCA 1/2 wild type triple negative breast cancer.

178. The method of claim 175 or 176, wherein the triple negative breast cancer is BRCA1/2 mutated triple negative breast cancer.

179. The method of any of claims 155-169, wherein the disease or condition is ovarian cancer.

180. The method of any of claims 155-169, wherein the disease or condition is cervical cancer.

181. The method of any of claims 155-169, wherein the disease or condition is lung cancer.

182. The method of any of claims 155-169, wherein the disease or condition is head and neck cancer.

183. The method of any of claims 155-169, wherein the disease or condition is renal cell carcinoma.

184. The method of any of claims 155-169, wherein the disease or condition is endometrial carcinoma.

185. The method of any of claims 155-169, wherein the disease or condition is a ROR1-expressing cancer.

186. A method of treating or reducing cancer in a subject in need thereof, comprising administering to the subject an effective amount of an antibody conjugate of any of claims 1 to 94 or the isolated antibody of any one of claims 95-134, or a pharmaceutical composition of any one of claims 144 to 154 wherein the effective amount of an antibody conjugate of any of claims 1 to 94 or the isolated antibody of any one of claims 95-134, or a pharmaceutical composition of any one of claims 144 to 154 treats cancer by activating anti-tumor immunity or protective immunity.

187. A method of reducing tumor size in a subject in need thereof, comprising administering to the subject an effective amount of an antibody conjugate of any of claims 1 to 94 or the isolated antibody of any one of claims 95-134, or a pharmaceutical composition of any one of claims 144 to 154.

188. The method of claim 187, wherein the tumor size is reduced by at least 25%.

189. The method of claim 187, wherein the tumor size is reduced by at least 50%.

190. A method of diagnosing cancer in a subject in need thereof, comprising:
(a) administering to the subject an effective amount of an antibody conjugate of any of claims 1 to 94 or the isolated antibody of any one of claims 95-134, or a pharmaceutical composition of any one of claims 144 to 154, wherein the antibody drug conjugate of any one of claims 1-94, the isolated antibody of any one of claims 95-134, or the antibody drug conjugate comprised in the pharmaceutical composition of any one of claims 144 to 154 optionally comprises a label; and
(b) detecting the antibody drug conjugate or detecting the label comprised in the antibody drug conjugate.

191. A method of diagnosing cancer in a subject in need thereof, comprising detecting the expression of ROR1 in a cell or tissue wherein the method comprises:
(a) administering to the subject an effective amount of an antibody conjugate of any of claims 1 to 94 or the isolated antibody of any one of claims 95-134, or a pharmaceutical composition of any one of claims 144 to 154, wherein the antibody drug conjugate of any one of claims 1-94, the isolated antibody of any one of claims 95-134, or the antibody drug conjugate comprised in the pharmaceutical composition of any one of claims 144 to 154 optionally comprises a label; and
(b) detecting the antibody drug conjugate or detecting the label comprised in the antibody drug conjugate.

192. A method of diagnosing cancer in a subject in need thereof, comprising
(a) detecting the expression of ROR1 in a cell or tissue of the subject; and
(b) administering to the subject an effective amount of an antibody conjugate of any of claims 1 to 94 or the isolated antibody of any one of claims 95-134, or a pharmaceutical composition of any one of claims 144 to 154.


SEQUENCE LISTING (1053 sequences)

SEQ ID NO: 1 - protein, 937 aa, Homo sapiens
  REGION 1..937: note = misc_feature - Human (hROR1) From UniProt Q01973
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 2 - protein, 937 aa, Macaca fascicularis
  REGION 1..937: note = misc_feature - Cynomolgus ROR1 From UniProt F6RUP2
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSPPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYIFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDAKQASLLGDANIHGHTESMISAEL

SEQ ID NO: 3 - protein, 937 aa, Mus musculus
  REGION 1..937: note = misc_feature - Murine ROR1 From UniProt Q9Z139
MHRPRRRGTRPPPLALLAALLLAARGADAQETELSVSAELVPTSSWNTSSEIDKGSYLTLDEPMNNITTSLGQTAELHCKVSGNPPPSIRWFKNDAPVVQEPRRISFRATNYGSRLRIRNLDTTDTGYFQCVATNGKKVVSTTGVLFVKFGPPPTASPGSSDEYEEDGFCQPYRGIACARFIGNRTVYMESLHMQGEIENQITAAFTMIGTSSHLSDKCSQFAIPSLCHYAFPYCDETSSVPKPRDLCRDECEVLENVLCQTEYIFARSNPMILMRLKLPNCEDLPQPESPEAANCIRIGIPMADPINKNHKCYNSTGVDYRGTVSVTKSGRQCQPWNSQYPHTHSFTALRFPELNGGHSYCRNPGNQKEAPWCFTLDENFKSDLCDIPACDSKDSKEKNKMEILYILVPSVAIPLAIAFLFFFICVCRNNQKSSSPPVQRQPKPVRGQNVEMSMLNAYKPKSKAKELPLSAVRFMEELGECTFGKIYKGHLYLPGMDHAQLVAIKTLKDYNNPQQWTEFQQEASLMAELHHPNIVCLLGAVTQEQPVCMLFEYMNQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSSLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRFPNYMFPSQGITPQGQIAGFIGPAIPQNQRFIPINGYPIPPGYAAFPAAHYQPAGPPRVIQHCPPPKSRSPSSASGSTSTGHVASLPSSGSNQEANVPLLPHMSIPNHPGGMGITVFGNKSQKPYKIDSKQSSLLGDSHIHGHTESMISAEV

SEQ ID NO: 4 - protein, 7 aa, synthetic construct
  REGION 1..7: note = Synthetic: 1987-C05; CDR-H1; Chothia
GFNISDY

SEQ ID NO: 5 - protein, 7 aa, synthetic construct
  REGION 1..7: note = Synthetic: 2188-D11; CDR-H1; Chothia
GFNIRDY

SEQ ID NO: 6 - protein, 7 aa, synthetic construct
  REGION 1..7: note = Synthetic: 2188-B04; CDR-H1; Chothia
GFNITWY

SEQ ID NO: 7 - protein, 7 aa, synthetic construct
  REGION 1..7: note = Synthetic: 2188-C09; CDR-H1; Chothia
GFNINSY

SEQ ID NO: 8 - protein, 7 aa, synthetic construct
  REGION 1..7: note = Synthetic: 2188-G03; CDR-H1; Chothia
GFNIVDY

SEQ ID NO: 9 - protein, 7 aa, synthetic construct
  REGION 1..7: note = Synthetic: 2188-E03; CDR-H1; Chothia
GFNIRDY

SEQ ID NO: 10 - protein, 7 aa, synthetic construct
  REGION 1..7: note = Synthetic: 2188-B11; CDR-H1; Chothia
GFNINRY

SEQ ID NO: 11 - protein, 7 aa, synthetic construct
  REGION 1..7: note = Synthetic: 2188-E07; CDR-H1; Chothia
GFNIGDY

SEQ ID NO: 12 - protein, 7 aa, synthetic construct
  REGION 1..7: note = Synthetic: 2188-B02; CDR-H1; Chothia
GFNISSY

SEQ ID NO: 13 - protein, 7 aa, synthetic construct
  REGION 1..7: note = Synthetic: 2188-C07; CDR-H1; Chothia
GFNIVRY

SEQ ID NO: 14 - protein, 7 aa, synthetic construct
  REGION 1..7: note = Synthetic: 2188-A03; CDR-H1; Chothia
GFNIRDY

SEQ ID NO: 15 - protein, 7 aa, synthetic construct
  REGION 1..7: note = Synthetic: 2188-F03; CDR-H1; Chothia
GFNISDY

SEQ ID NO: 16 - protein, 7 aa, synthetic construct
  REGION 1..7: note = Synthetic: 2188-D03; CDR-H1; Chothia
GFNISDY

SEQ ID NO: 17 - protein, 7 aa, synthetic construct
  REGION 1..7: note = Synthetic: 2188-C04; CDR-H1; Chothia
GFNINSY

SEQ ID NO: 18 - protein, 7 aa, synthetic construct
  REGION 1..7: note = Synthetic: 2188-D10; CDR-H1; Chothia
GFNISDY

SEQ ID NO: 19 - protein, 7 aa, synthetic construct
  REGION 1..7: note = Synthetic: 2188-A06; CDR-H1; Chothia
GFNIKSY

SEQ ID NO: 20 - protein, 7 aa, synthetic construct
  REGION 1..7: note = Synthetic: 2188-C11; CDR-H1; Chothia
GFNIKSY

SEQ ID NO: 21 - protein, 7 aa, synthetic construct
  REGION 1..7: note = Synthetic: 2188-F01; CDR-H1; Chothia
GFNIRYY

SEQ ID NO: 22 - protein, 7 aa, synthetic construct
  REGION 1..7: note = Synthetic: 2188-E11; CDR-H1; Chothia
GFNISHY

SEQ ID NO: 23 - protein, 7 aa, synthetic construct
  REGION 1..7: note = Synthetic: 2188-A07; CDR-H1; Chothia
GFNIHHY

SEQ ID NO: 24 - protein, 7 aa, synthetic construct
  REGION 1..7: note = Synthetic: 2188-C01; CDR-H1; Chothia
GFNIISY

SEQ ID NO: 25 - protein, 7 aa, synthetic construct
  REGION 1..7: note = Synthetic: 2188-F08; CDR-H1; Chothia
GFNIPDF

SEQ ID NO: 26 - protein, 7 aa, synthetic construct
  REGION 1..7: note = Synthetic: 2188-E04; CDR-H1; Chothia
GFNISDY

SEQ ID NO: 27 - protein, 7 aa, synthetic construct
  REGION 1..7: note = Synthetic: 2188-B01; CDR-H1; Chothia
GFNINSH

SEQ ID NO: 28 - protein, 7 aa, synthetic construct
  REGION 1..7: note = Synthetic: 2188-F11; CDR-H1; Chothia
GFNITGY

SEQ ID NO: 29 - protein, 7 aa, synthetic construct
  REGION 1..7: note = Synthetic: 2188-B08; CDR-H1; Chothia
GFNIDSY

SEQ ID NO: 30 - protein, 7 aa, synthetic construct
  REGION 1..7: note = Synthetic: 2188-C10; CDR-H1; Chothia
GFNINSY

SEQ ID NO: 31 - protein, 7 aa, synthetic construct
  REGION 1..7: note = Synthetic: 2188-C02; CDR-H1; Chothia
GFNIERY

SEQ ID NO: 32 - protein, 7 aa, synthetic construct
  REGION 1..7: note = Synthetic: 2188-B07; CDR-H1; Chothia
GFNIHSH

SEQ ID NO: 33 - protein, 7 aa, synthetic construct
  REGION 1..7: note = Synthetic: 2188-A11; CDR-H1; Chothia
GFNISDY

SEQ ID NO: 34 - protein, 7 aa, synthetic construct
  REGION 1..7: note = Synthetic: 2188-D01; CDR-H1; Chothia
GFNIKSY

SEQ ID NO: 35 - protein, 7 aa, synthetic construct
  REGION 1..7: note = Synthetic: 2188-E09; CDR-H1; Chothia
GFNISDF

SEQ ID NO: 36 - protein, 7 aa, synthetic construct
  REGION 1..7: note = Synthetic: 2188-E06; CDR-H1; Chothia
GFNIGDY

SEQ ID NO: 37 - protein, 7 aa, synthetic construct
  REGION 1..7: note = Synthetic: 2188-B03; CDR-H1; Chothia
GFNISSY

SEQ ID NO: 38 - protein, 7 aa, synthetic construct
  REGION 1..7: note = Synthetic: 2188-F06; CDR-H1; Chothia
GFNILDY

SEQ ID NO: 39 - protein, 7 aa, synthetic construct
  REGION 1..7: note = Synthetic: 2188-D02; CDR-H1; Chothia
GFNISDY

SEQ ID NO: 40 - protein, 7 aa, synthetic construct
  REGION 1..7: note = Synthetic: 2188-B06; CDR-H1; Chothia
GFNIHSY

SEQ ID NO: 41 - protein, 7 aa, synthetic construct
  REGION 1..7: note = Synthetic: 2188-D09; CDR-H1; Chothia
GFNIRDY

SEQ ID NO: 42 - protein, 7 aa, synthetic construct
  REGION 1..7: note = Synthetic: 2188-F02; CDR-H1; Chothia
GFNISDY

SEQ ID NO: 43 - protein, 7 aa, synthetic construct
  REGION 1..7: note = Synthetic: 2188-E10; CDR-H1; Chothia
GFNIGDF

SEQ ID NO: 44 - protein, 7 aa, synthetic construct
  REGION 1..7: note = Synthetic: 2188-A09; CDR-H1; Chothia
GFNIINY

SEQ ID NO: 45 - protein, 7 aa, synthetic construct
  REGION 1..7: note = Synthetic: 2188-D04; CDR-H1; Chothia
GFNISDY

SEQ ID NO: 46 - protein, 7 aa, synthetic construct
  REGION 1..7: note = Synthetic: 2188-A05; CDR-H1; Chothia
GFNIKRY

SEQ ID NO: 47 - protein, 7 aa, synthetic construct
  REGION 1..7: note = Synthetic: 2188-E01; CDR-H1; Chothia
GFNIGDY

SEQ ID NO: 48 - protein, 7 aa, synthetic construct
  REGION 1..7: note = Synthetic: 2188-G01; CDR-H1; Chothia
GFNISDY

SEQ ID NO: 49 - protein, 7 aa, synthetic construct
  REGION 1..7: note = Synthetic: 2188-B09; CDR-H1; Chothia
GFNINSY

SEQ ID NO: 50 - protein, 7 aa, synthetic construct
  REGION 1..7: note = Synthetic: 2188-F07; CDR-H1; Chothia
GFNISDY

SEQ ID NO: 51 - protein, 7 aa, synthetic construct
  REGION 1..7: note = Synthetic: 2188-D08; CDR-H1; Chothia
GFNIRDY

SEQ ID NO: 52 - protein, 7 aa, synthetic construct
  REGION 1..7: note = Synthetic: 2188-D05; CDR-H1; Chothia
GFNISEY

SEQ ID NO: 53 - protein, 7 aa, synthetic construct
  REGION 1..7: note = Synthetic: 2188-C03; CDR-H1; Chothia
GFNINSY

SEQ ID NO: 54 - protein, 7 aa, synthetic construct
  REGION 1..7: note = Synthetic: 2188-E08; CDR-H1; Chothia
GFNIIDF

SEQ ID NO: 55 - protein, 7 aa, synthetic construct
  REGION 1..7: note = Synthetic: 2188-C06; CDR-H1; Chothia
GFNINSY

SEQ ID NO: 56 - protein, 7 aa, synthetic construct
  REGION 1..7: note = Synthetic: 2188-A08; CDR-H1; Chothia
GFNIHNF

SEQ ID NO: 57 - protein, 7 aa, synthetic construct
  REGION 1..7: note = Synthetic: 2188-B10; CDR-H1; Chothia
GFNINSY

SEQ ID NO: 58 - protein, 7 aa, synthetic construct
  REGION 1..7: note = Synthetic: 2188-G02; CDR-H1; Chothia
GFNIVGY

SEQ ID NO: 59 - protein, 7 aa, synthetic construct
  REGION 1..7: note = Synthetic: 2188-C05; CDR-H1; Chothia
GFNINSF

SEQ ID NO: 60 - protein, 7 aa, synthetic construct
  REGION 1..7: note = Synthetic: 2188-A10; CDR-H1; Chothia
GFNINSY

SEQ ID NO: 61 - protein, 7 aa, synthetic construct
  REGION 1..7: note = Synthetic: 2188-G04; CDR-H1; Chothia
GFNIFDY

SEQ ID NO: 62 - protein, 7 aa, synthetic construct
  REGION 1..7: note = Synthetic: 2188-C08; CDR-H1; Chothia
GFNIKSH

SEQ ID NO: 63 - protein, 7 aa, synthetic construct
  REGION 1..7: note = Synthetic: 2188-E05; CDR-H1; Chothia
GFNISDY

SEQ ID NO: 64 - protein, 7 aa, synthetic construct
  REGION 1..7: note = Synthetic: 2188-A02; CDR-H1; Chothia
GFNIKSY

SEQ ID NO: 65 - protein, 7 aa, synthetic construct
  REGION 1..7: note = Synthetic: 2188-F04; CDR-H1; Chothia
GFNISDF

SEQ ID NO: 66 - protein, 7 aa, synthetic construct
  REGION 1..7: note = Synthetic: 2188-F05; CDR-H1; Chothia
GFNISDY

SEQ ID NO: 67 - protein, 7 aa, synthetic construct
  REGION 1..7: note = Synthetic: 2188-D07; CDR-H1; Chothia
GFNISDF

SEQ ID NO: 68 - protein, 7 aa, synthetic construct
  REGION 1..7: note = Synthetic: 2188-E02; CDR-H1; Chothia
GFNIPDY

SEQ ID NO: 69 - protein, 7 aa, synthetic construct
  REGION 1..7: note = Synthetic: 2188-D06; CDR-H1; Chothia
GFNIRDY

SEQ ID NO: 70 - protein, 7 aa, synthetic construct
  REGION 1..7: note = Synthetic: 2188-F09; CDR-H1; Chothia
GFNIRDY

SEQ ID NO: 71 - protein, 7 aa, synthetic construct
  REGION 1..7: note = Synthetic: 2188-F10; CDR-H1; Chothia
GFNISVF

SEQ ID NO: 72 - protein, 7 aa, synthetic construct
  REGION 1..7: note = Synthetic: 2188-B05; CDR-H1; Chothia
GFNIPDY

SEQ ID NO: 73 - protein, 7 aa, synthetic construct
  REGION 1..7: note = Synthetic: 1943-C02; CDR-H1; Chothia
GFNINDY

SEQ ID NO: 74 - protein, 7 aa, synthetic construct
  REGION 1..7: note = Synthetic: 2193-D04; CDR-H1; Chothia
GFNINGF

SEQ ID NO: 75 - protein, 7 aa, synthetic construct
  REGION 1..7: note = Synthetic: 2193-E10; CDR-H1; Chothia
GFNIYDY

SEQ ID NO: 76 - protein, 7 aa, synthetic construct
  REGION 1..7: note = Synthetic: 2193-E06; CDR-H1; Chothia
GFNIDDY

SEQ ID NO: 77 - protein, 7 aa, synthetic construct
  REGION 1..7: note = Synthetic: 2193-E04; CDR-H1; Chothia
GFNINDR

SEQ ID NO: 78 - protein, 7 aa, synthetic construct
  REGION 1..7: note = Synthetic: 2193-B09; CDR-H1; Chothia
GFNINNS

SEQ ID NO: 79 - protein, 7 aa, synthetic construct
  REGION 1..7: note = Synthetic: 2193-D11; CDR-H1; Chothia
GFNINAY

SEQ ID NO: 80 - protein, 7 aa, synthetic construct
  REGION 1..7: note = Synthetic: 2193-B02; CDR-H1; Chothia
GFNIKNS

SEQ ID NO: 81 - protein, 7 aa, synthetic construct
  REGION 1..7: note = Synthetic: 2193-D05; CDR-H1; Chothia
GFNINDH

SEQ ID NO: 82 - protein, 7 aa, synthetic construct
  REGION 1..7: note = Synthetic: 2193-E11; CDR-H1; Chothia
GFNINDY

SEQ ID NO: 83 - protein, 7 aa, synthetic construct
  REGION 1..7: note = Synthetic: 2193-D06; CDR-H1; Chothia
GFNIRDY

SEQ ID NO: 84 - protein, 7 aa, synthetic construct
  REGION 1..7: note = Synthetic: 2193-C02; CDR-H1; Chothia
GFNISDY

SEQ ID NO: 85 - protein, 7 aa, synthetic construct
  REGION 1..7: note = Synthetic: 2193-B03; CDR-H1; Chothia
GFNIRDY

SEQ ID NO: 86 - protein, 7 aa, synthetic construct
  REGION 1..7: note = Synthetic: 2193-A02; CDR-H1; Chothia
GFNIHAS

SEQ ID NO: 87 - protein, 7 aa, synthetic construct
  REGION 1..7: note = Synthetic: 2193-E05; CDR-H1; Chothia
GFNIKRS

SEQ ID NO: 88 - protein, 7 aa, synthetic construct
  REGION 1..7: note = Synthetic: 2193-A06; CDR-H1; Chothia
GFNINAY

SEQ ID NO: 89 - protein, 7 aa, synthetic construct
  REGION 1..7: note = Synthetic: 2193-C04; CDR-H1; Chothia
GFNINTS

SEQ ID NO: 90 - protein, 7 aa, synthetic construct
  REGION 1..7: note = Synthetic: 2193-E08; CDR-H1; Chothia
GFNINEY

SEQ ID NO: 91 - protein, 7 aa, synthetic construct
  REGION 1..7: note = Synthetic: 2193-B10; CDR-H1; Chothia
GFNIADY

SEQ ID NO: 92 - protein, 7 aa, synthetic construct
  REGION 1..7: note = Synthetic: 2193-D08; CDR-H1; Chothia
GFNIKRS

SEQ ID NO: 93 - protein, 7 aa, synthetic construct
  REGION 1..7: note = Synthetic: 2193-B08; CDR-H1; Chothia
GFNIKDY

SEQ ID NO: 94 - protein, 7 aa, synthetic construct
  REGION 1..7: note = Synthetic: 2193-C05; CDR-H1; Chothia
GFNIKHS

SEQ ID NO: 95 - protein, 7 aa, synthetic construct
  REGION 1..7: note = Synthetic: 2193-D10; CDR-H1; Chothia
GFNINVS

SEQ ID NO: 96 - protein, 7 aa, synthetic construct
  REGION 1..7: note = Synthetic: 2193-D03; CDR-H1; Chothia
GFNINDH

SEQ ID NO: 97 - protein, 7 aa, synthetic construct
  REGION 1..7: note = Synthetic: 2193-A09; CDR-H1; Chothia
GFNIGKH

SEQ ID NO: 98 - protein, 7 aa, synthetic construct
  REGION 1..7: note = Synthetic: 2193-A10; CDR-H1; Chothia
GFNIGDY

SEQ ID NO: 99 - protein, 7 aa, synthetic construct
  REGION 1..7: note = Synthetic: 2193-E07; CDR-H1; Chothia
GFNINKV

SEQ ID NO: 100 - protein, 7 aa, synthetic construct
  REGION 1..7: note = Synthetic: 2193-A07; CDR-H1; Chothia
GFNINAS

SEQ ID NO: 101 - protein, 7 aa, synthetic construct
  REGION 1..7: note = Synthetic: 2193-E03; CDR-H1; Chothia
GFNITAS

SEQ ID NO: 102 - protein, 7 aa, synthetic construct
  REGION 1..7: note = Synthetic: 2193-C08; CDR-H1; Chothia
GFNINHY

SEQ ID NO: 103 - protein, 7 aa, synthetic construct
  REGION 1..7: note = Synthetic: 2193-B11; CDR-H1; Chothia
GFNITGS

SEQ ID NO: 104 - protein, 7 aa, synthetic construct
  REGION 1..7: note = Synthetic: 2193-A03; CDR-H1; Chothia
GFNIANY

SEQ ID NO: 105 - protein, 7 aa, synthetic construct
  REGION 1..7: note = Synthetic: 2193-D02; CDR-H1; Chothia
GFNIKDY

SEQ ID NO: 106 - protein, 7 aa, synthetic construct
  REGION 1..7: note = Synthetic: 2193-B06; CDR-H1; Chothia
GFNIDGY

SEQ ID NO: 107 - protein, 7 aa, synthetic construct
  REGION 1..7: note = Synthetic: 2193-B01; CDR-H1; Chothia
GFNIGGY

SEQ ID NO: 108 - protein, 7 aa, synthetic construct
  REGION 1..7: note = Synthetic: 2193-D09; CDR-H1; Chothia
GFNINGY

SEQ ID NO: 109 - protein, 7 aa, synthetic construct
  REGION 1..7: note = Synthetic: 2193-E09; CDR-H1; Chothia
GFNIGAY

SEQ ID NO: 110 - protein, 7 aa, synthetic construct
  REGION 1..7: note = Synthetic: 2193-B07; CDR-H1; Chothia
GFNINNS

SEQ ID NO: 111 - protein, 7 aa, synthetic construct
  REGION 1..7: note = Synthetic: 2193-C06; CDR-H1; Chothia
GFNIIVH

SEQ ID NO: 112 - protein, 7 aa, synthetic construct
  REGION 1..7: note = Synthetic: 2193-B05; CDR-H1; Chothia
GFNINQY

SEQ ID NO: 113 - protein, 7 aa, synthetic construct
  REGION 1..7: note = Synthetic: 2193-A11; CDR-H1; Chothia
GFNIDAY

SEQ ID NO: 114 - protein, 7 aa, synthetic construct
  REGION 1..7: note = Synthetic: 2193-E02; CDR-H1; Chothia
GFNIKDY

SEQ ID NO: 115 - protein, 7 aa, synthetic construct
  REGION 1..7: note = Synthetic: 2193-A08; CDR-H1; Chothia
GFNIIDS

SEQ ID NO: 116 - protein, 7 aa, synthetic construct
  REGION 1..7: note = Synthetic: 2193-C11; CDR-H1; Chothia
GFNIADS

SEQ ID NO: 117 - protein, 7 aa, synthetic construct
  REGION 1..7: note = Synthetic: 2193-A01; CDR-H1; Chothia
GFNINDH

SEQ ID NO: 118 - protein, 7 aa, synthetic construct
  REGION 1..7: note = Synthetic: 2193-D07; CDR-H1; Chothia
GFNIIDY

SEQ ID NO: 119 - protein, 7 aa, synthetic construct
  REGION 1..7: note = Synthetic: 2193-A05; CDR-H1; Chothia
GFNIKGY

SEQ ID NO: 120 - protein, 7 aa, synthetic construct
  REGION 1..7: note = Synthetic: 2193-C03; CDR-H1; Chothia
GFNINYS

SEQ ID NO: 121 - protein, 7 aa, synthetic construct
  REGION 1..7: note = Synthetic: 2193-C09; CDR-H1; Chothia
GFNINHS

SEQ ID NO: 122 - protein, 7 aa, synthetic construct
  REGION 1..7: note = Synthetic: 2193-D01; CDR-H1; Chothia
GFNINDS

SEQ ID NO: 123 - protein, 7 aa, synthetic construct
  REGION 1..7: note = Synthetic: 2193-E01; CDR-H1; Chothia
GFNIKGK

SEQ ID NO: 124 - protein, 7 aa, synthetic construct
  REGION 1..7: note = Synthetic: 2193-C07; CDR-H1; Chothia
GFNIKDQ

SEQ ID NO: 125 - protein, 7 aa, synthetic construct
  REGION 1..7: note = Synthetic: 2193-B04; CDR-H1; Chothia
GFNISNS

SEQ ID NO: 126 - protein, 7 aa, synthetic construct
  REGION 1..7: note = Synthetic: 2193-C10; CDR-H1; Chothia
GFNINNA

SEQ ID NO: 127 - protein, 7 aa, synthetic construct
  REGION 1..7: note = Synthetic: 2193-C01; CDR-H1; Chothia
GLNINDH

SEQ ID NO: 128 - protein, 7 aa, synthetic construct
  REGION 1..7: note = Synthetic: 2193-A04; CDR-H1; Chothia
GFNIISY

SEQ ID NO: 129 - protein, 7 aa, synthetic construct
  REGION 1..7: note = Synthetic: 1944-A07; CDR-H1; Chothia
GFNITGY

SEQ ID NO: 130 - protein, 7 aa, synthetic construct
  REGION 1..7: note = Synthetic: 2194-A02; CDR-H1; Chothia
GFNITGY

SEQ ID NO: 131 - protein, 7 aa, synthetic construct
  REGION 1..7: note = Synthetic: 2194-A05; CDR-H1; Chothia
GFNIKEY

SEQ ID NO: 132 - protein, 7 aa, synthetic construct
  REGION 1..7: note = Synthetic: 2194-B04; CDR-H1; Chothia
GFNITGY

SEQ ID NO: 133 - protein, 7 aa, synthetic construct
  REGION 1..7: note = Synthetic: 2194-A09; CDR-H1; Chothia
GFNITGY

SEQ ID NO: 134 - protein, 7 aa, synthetic construct
  REGION 1..7: note = Synthetic: 2194-A01; CDR-H1; Chothia
GFNITGY

SEQ ID NO: 135 - protein, 7 aa, synthetic construct
  REGION 1..7: note = Synthetic: 2194-B03; CDR-H1; Chothia
GFNITSY

SEQ ID NO: 136 - protein, 7 aa, synthetic construct
  REGION 1..7: note = Synthetic: 2194-B02; CDR-H1; Chothia
GFNISGY

SEQ ID NO: 137 - protein, 7 aa, synthetic construct
  REGION 1..7: note = Synthetic: 2194-A03; CDR-H1; Chothia
GFNITGY

SEQ ID NO: 138 - protein, 7 aa, synthetic construct
  REGION 1..7: note = Synthetic: 2194-A11; CDR-H1; Chothia
GFNITQY

SEQ ID NO: 139 - protein, 7 aa, synthetic construct
  REGION 1..7: note = Synthetic: 2194-A08; CDR-H1; Chothia
GFNITGY

SEQ ID NO: 140 - protein, 7 aa, synthetic construct
  REGION 1..7: note = Synthetic: 2194-A04; CDR-H1; Chothia
GFNITGY

SEQ ID NO: 141 - protein, 7 aa, synthetic construct
  REGION 1..7: note = Synthetic: 2194-A10; CDR-H1; Chothia
GFNITQY

SEQ ID NO: 142 - protein, 7 aa, synthetic construct
  REGION 1..7: note = Synthetic: 2194-A07; CDR-H1; Chothia
GFNFTDY

SEQ ID NO: 143 - protein, 7 aa, synthetic construct
  REGION 1..7: note = Synthetic: 2194-B01; CDR-H1; Chothia
GFNIASY

SEQ ID NO: 144 - protein, 7 aa, synthetic construct
  REGION 1..7: note = Synthetic: 2194-A06; CDR-H1; Chothia
GFNITDY

SEQ ID NO: 145 - protein, 7 aa, synthetic construct
  REGION 1..7: note = Synthetic: 2196-C01; CDR-H1; Chothia
GFNIIGY

SEQ ID NO: 146 - protein, 7 aa, synthetic construct
  REGION 1..7: note = Synthetic: 2196-A02; CDR-H1; Chothia
GFNITGY

SEQ ID NO: 147 - protein, 7 aa, synthetic construct
  REGION 1..7: note = Synthetic: 2196-B03; CDR-H1; Chothia
GFNITGY

SEQ ID NO: 148 - protein, 7 aa, synthetic construct
  REGION 1..7: note = Synthetic: 2196-A05; CDR-H1; Chothia
GFNITNY

SEQ ID NO: 149 - protein, 7 aa, synthetic construct
  REGION 1..7: note = Synthetic: 2196-C02; CDR-H1; Chothia
GFNITGY

SEQ ID NO: 150 - protein, 7 aa, synthetic construct
  REGION 1..7: note = Synthetic: 2196-B11; CDR-H1; Chothia
GFNITGY

SEQ ID NO: 151 - protein, 7 aa, synthetic construct
  REGION 1..7: note = Synthetic: 2196-B08; CDR-H1; Chothia
GFNITGY

SEQ ID NO: 152 - protein, 7 aa, synthetic construct
  REGION 1..7: note = Synthetic: 2196-A04; CDR-H1; Chothia
GFNISPY

SEQ ID NO: 153 - protein, 7 aa, synthetic construct
  REGION 1..7: note = Synthetic: 2196-A03; CDR-H1; Chothia
GFNITQY

SEQ ID NO: 154 - protein, 7 aa, synthetic construct
  REGION 1..7: note = Synthetic: 2196-B07; CDR-H1; Chothia
GFNITGY

SEQ ID NO: 155 - protein, 7 aa, synthetic construct
  REGION 1..7: note = Synthetic: 2196-A06; CDR-H1; Chothia
GFNITGY

SEQ ID NO: 156 - protein, 7 aa, synthetic construct
  REGION 1..7: note = Synthetic: 2196-B05; CDR-H1; Chothia
GFNITGY

SEQ ID NO: 157 - protein, 7 aa, synthetic construct
  REGION 1..7: note = Synthetic: 2196-A01; CDR-H1; Chothia
GFNITGY

SEQ ID NO: 158 - protein, 7 aa, synthetic construct
  REGION 1..7: note = Synthetic: 2196-B01; CDR-H1; Chothia
GFNITGY

SEQ ID NO: 159 - protein, 7 aa, synthetic construct
  REGION 1..7: note = Synthetic: 2196-A09; CDR-H1; Chothia
GFNITGY

SEQ ID NO: 160 - protein, 7 aa, synthetic construct
  REGION 1..7: note = Synthetic: 2196-A08; CDR-H1; Chothia
GFNITGY

SEQ ID NO: 161 - protein, 7 aa, synthetic construct
  REGION 1..7: note = Synthetic: 2196-A10; CDR-H1; Chothia
GFNITGY

SEQ ID NO: 162 - protein, 7 aa, synthetic construct
  REGION 1..7: note = Synthetic: 2196-B06; CDR-H1; Chothia
GFNITGY

SEQ ID NO: 163 - protein, 7 aa, synthetic construct
  REGION 1..7: note = Synthetic: 2196-B09; CDR-H1; Chothia
GFNITEY

SEQ ID NO: 164 - protein, 7 aa, synthetic construct
  REGION 1..7: note = Synthetic: 2196-C03; CDR-H1; Chothia
GFNITGY

SEQ ID NO: 165 - protein, 7 aa, synthetic construct
  REGION 1..7: note = Synthetic: 2196-C04; CDR-H1; Chothia
GFNITGY

SEQ ID NO: 166 - protein, 7 aa, synthetic construct
  REGION 1..7: note = Synthetic: 2196-A07; CDR-H1; Chothia
GFNITGY

SEQ ID NO: 167 - protein, 7 aa, synthetic construct
  REGION 1..7: note = Synthetic: 2196-A11; CDR-H1; Chothia
GFNITGY

SEQ ID NO: 168 - protein, 7 aa, synthetic construct
  REGION 1..7: note = Synthetic: 2196-B02; CDR-H1; Chothia
GFNITQY

SEQ ID NO: 169 - protein, 7 aa, synthetic construct
  REGION 1..7: note = Synthetic: 2196-B04; CDR-H1; Chothia
GFNITEY

SEQ ID NO: 170 - protein, 7 aa, synthetic construct
  REGION 1..7: note = Synthetic: 2196-B10; CDR-H1; Chothia
GFNITGY

SEQ ID NO: 171 - protein, 5 aa, synthetic construct
  REGION 1..5: note = Synthetic: 1987-C05; CDR-H1; Kabat
DYDIH

SEQ ID NO: 172 - protein, 5 aa, synthetic construct
  REGION 1..5: note = Synthetic: 2188-D11; CDR-H1; Kabat
DYDIH

SEQ ID NO: 173 - protein, 5 aa, synthetic construct
  REGION 1..5: note = Synthetic: 2188-B04; CDR-H1; Kabat
WYDIH

SEQ ID NO: 174 - protein, 5 aa, synthetic construct
  REGION 1..5: note = Synthetic: 2188-C09; CDR-H1; Kabat
SYDIH

SEQ ID NO: 175 - protein, 5 aa, synthetic construct
  REGION 1..5: note = Synthetic: 2188-G03; CDR-H1; Kabat
DYDIH

SEQ ID NO: 176 - protein, 5 aa, synthetic construct
  REGION 1..5: note = Synthetic: 2188-E03; CDR-H1; Kabat
DYDIH

SEQ ID NO: 177 - protein, 5 aa, synthetic construct
  REGION 1..5: note = Synthetic: 2188-B11; CDR-H1; Kabat
RYDIH

SEQ ID NO: 178 - protein, 5 aa, synthetic construct
  REGION 1..5: note = Synthetic: 2188-E07; CDR-H1; Kabat
DYGIH

SEQ ID NO: 179 - protein, 5 aa, synthetic construct
  REGION 1..5: note = Synthetic: 2188-B02; CDR-H1; Kabat
SYDIH

SEQ ID NO: 180 - protein, 5 aa, synthetic construct
  REGION 1..5: note = Synthetic: 2188-C07; CDR-H1; Kabat
RYDIH

SEQ ID NO: 181 - protein, 5 aa, synthetic construct
  REGION 1..5: note = Synthetic: 2188-A03; CDR-H1; Kabat
DYDIH

SEQ ID NO: 182 - protein, 5 aa, synthetic construct
  REGION 1..5: note = Synthetic: 2188-F03; CDR-H1; Kabat
DYDIH

SEQ ID NO: 183 - protein, 5 aa, synthetic construct
  REGION 1..5: note = Synthetic: 2188-D03; CDR-H1; Kabat
DYDIH

SEQ ID NO: 184 - protein, 5 aa, synthetic construct
  REGION 1..5: note = Synthetic: 2188-C04; CDR-H1; Kabat
SYDIH

SEQ ID NO: 185 - protein, 5 aa, synthetic construct
  REGION 1..5: note = Synthetic: 2188-D10; CDR-H1; Kabat
DYDIH

SEQ ID NO: 186 - protein, 5 aa, synthetic construct
  REGION 1..5: note = Synthetic: 2188-A06; CDR-H1; Kabat
SYDIH

SEQ ID NO: 187 - protein, 5 aa, synthetic construct
  REGION 1..5: note = Synthetic: 2188-C11; CDR-H1; Kabat
SYDIH

SEQ ID NO: 188 - protein, 5 aa, synthetic construct
  REGION 1..5: note = Synthetic: 2188-F01; CDR-H1; Kabat
YYDIH

SEQ ID NO: 189 - protein, 5 aa, synthetic construct
  REGION 1..5: note = Synthetic: 2188-E11; CDR-H1; Kabat
HYDIH

SEQ ID NO: 190 - protein, 5 aa, synthetic construct
  REGION 1..5: note = Synthetic: 2188-A07; CDR-H1; Kabat
HYDIH

SEQ ID NO: 191 - protein, 5 aa, synthetic construct
  REGION 1..5: note = Synthetic: 2188-C01; CDR-H1; Kabat
SYDIH

SEQ ID NO: 192 - protein, 5 aa, synthetic construct
  REGION 1..5: note = Synthetic: 2188-F08; CDR-H1; Kabat
DFEIH

SEQ ID NO: 193 - protein, 5 aa, synthetic construct
  REGION 1..5: note = Synthetic: 2188-E04; CDR-H1; Kabat
DYDIH

SEQ ID NO: 194 - protein, 5 aa, synthetic construct
  REGION 1..5: note = Synthetic: 2188-B01; CDR-H1; Kabat
SHDIH

SEQ ID NO: 195 - protein, 5 aa, synthetic construct
  REGION 1..5: note = Synthetic: 2188-F11; CDR-H1; Kabat
GYDIH

SEQ ID NO: 196 - protein, 5 aa, synthetic construct
  REGION 1..5: note = Synthetic: 2188-B08; CDR-H1; Kabat
SYDIH

SEQ ID NO: 197 - protein, 5 aa, synthetic construct
  REGION 1..5: note = Synthetic: 2188-C10; CDR-H1; Kabat
SYDIH

SEQ ID NO: 198 - protein, 5 aa, synthetic construct
  REGION 1..5: note = Synthetic: 2188-C02; CDR-H1; Kabat
RYDIH

SEQ ID NO: 199 - protein, 5 aa, synthetic construct
  REGION 1..5: note = Synthetic: 2188-B07; CDR-H1; Kabat
SHDIH

SEQ ID NO: 200 - protein, 5 aa, synthetic construct
  REGION 1..5: note = Synthetic: 2188-A11; CDR-H1; Kabat
DYDIH

SEQ ID NO: 201 - protein, 5 aa, synthetic construct
  REGION 1..5: note = Synthetic: 2188-D01; CDR-H1; Kabat
SYDIH

SEQ ID NO: 202 - protein, 5 aa, synthetic construct
  REGION 1..5: note = Synthetic: 2188-E09; CDR-H1; Kabat
DFDIH

SEQ ID NO: 203 - protein, 5 aa, synthetic construct
  REGION 1..5: note = Synthetic: 2188-E06; CDR-H1; Kabat
DYDIH

SEQ ID NO: 204 - protein, 5 aa, synthetic construct
  REGION 1..5: note = Synthetic: 2188-B03; CDR-H1; Kabat
SYDIH

SEQ ID NO: 205 - protein, 5 aa, synthetic construct
  REGION 1..5: note = Synthetic: 2188-F06; CDR-H1; Kabat
DYDIH

SEQ ID NO: 206 - protein, 5 aa, synthetic construct
  REGION 1..5: note = Synthetic: 2188-D02; CDR-H1; Kabat
DYDIH

SEQ ID NO: 207 - protein, 5 aa, synthetic construct
  REGION 1..5: note = Synthetic: 2188-B06; CDR-H1; Kabat
SYDIH

SEQ ID NO: 208 - protein, 5 aa, synthetic construct
  REGION 1..5: note = Synthetic: 2188-D09; CDR-H1; Kabat
DYDIH

SEQ ID NO: 209 - protein, 5 aa, synthetic construct
  REGION 1..5: note = Synthetic: 2188-F02; CDR-H1; Kabat
DYDIH

SEQ ID NO: 210 - protein, 5 aa, synthetic construct
  REGION 1..5: note = Synthetic: 2188-E10; CDR-H1; Kabat
DFDIH

SEQ ID NO: 211 - protein, 5 aa, synthetic construct
  REGION 1..5: note = Synthetic: 2188-A09; CDR-H1; Kabat
NYDIH

SEQ ID NO: 212 - protein, 5 aa, synthetic construct
  REGION 1..5: note = Synthetic: 2188-D04; CDR-H1; Kabat
DYDIH

SEQ ID NO: 213 - protein, 5 aa, synthetic construct
  REGION 1..5: note = Synthetic: 2188-A05; CDR-H1; Kabat
RYDIH

SEQ ID NO: 214 - protein, 5 aa, synthetic construct
  REGION 1..5: note = Synthetic: 2188-E01; CDR-H1; Kabat
DYDIH

SEQ ID NO: 215 - protein, 5 aa, synthetic construct
  REGION 1..5: note = Synthetic: 2188-G01; CDR-H1; Kabat
DYDIH

SEQ ID NO: 216 - protein, 5 aa, synthetic construct
  REGION 1..5: note = Synthetic: 2188-B09; CDR-H1; Kabat
SYDIH

SEQ ID NO: 217 - protein, 5 aa, synthetic construct
  REGION 1..5: note = Synthetic: 2188-F07; CDR-H1; Kabat
DYDIH

SEQ ID NO: 218 - protein, 5 aa, synthetic construct
  REGION 1..5: note = Synthetic: 2188-D08; CDR-H1; Kabat
DYDIH

SEQ ID NO: 219 - protein, 5 aa, synthetic construct
  REGION 1..5: note = Synthetic: 2188-D05; CDR-H1; Kabat
EYDIH

SEQ ID NO: 220 - protein, 5 aa, synthetic construct
  REGION 1..5: note = Synthetic: 2188-C03; CDR-H1; Kabat
SYDIH

SEQ ID NO: 221 - protein, 5 aa, synthetic construct
  REGION 1..5: note = Synthetic: 2188-E08; CDR-H1; Kabat
DFDIH

SEQ ID NO: 222 - protein, 5 aa, synthetic construct
  REGION 1..5: note = Synthetic: 2188-C06; CDR-H1; Kabat
SYDIH

SEQ ID NO: 223 - protein, 5 aa, synthetic construct
  REGION 1..5: note = Synthetic: 2188-A08; CDR-H1; Kabat
NFDIH

SEQ ID NO: 224 - protein, 5 aa, synthetic construct
  REGION 1..5: note = Synthetic: 2188-B10; CDR-H1; Kabat
SYDIH

SEQ ID NO: 225 - protein, 5 aa, synthetic construct
  REGION 1..5: note = Synthetic: 2188-G02; CDR-H1; Kabat
GYDIH

SEQ ID NO: 226 - protein, 5 aa, synthetic construct
  REGION 1..5: note = Synthetic: 2188-C05; CDR-H1; Kabat
SFDIH

SEQ ID NO: 227 - protein, 5 aa, synthetic construct
  REGION 1..5: note = Synthetic: 2188-A10; CDR-H1; Kabat
SYDIH

SEQ ID NO: 228 - protein, 5 aa, synthetic construct
  REGION 1..5: note = Synthetic: 2188-G04; CDR-H1; Kabat
DYDIH

SEQ ID NO: 229 - protein, 5 aa, synthetic construct
  REGION 1..5: note = Synthetic: 2188-C08; CDR-H1; Kabat
SHDIH

SEQ ID NO: 230 - protein, 5 aa, synthetic construct
  REGION 1..5: note = Synthetic: 2188-E05; CDR-H1; Kabat
DYDIH

SEQ ID NO: 231 - protein, 5 aa, synthetic construct
  REGION 1..5: note = Synthetic: 2188-A02; CDR-H1; Kabat
SYDIH

SEQ ID NO: 232 - protein, 5 aa, synthetic construct
  REGION 1..5: note = Synthetic: 2188-F04; CDR-H1; Kabat
DFDIH

SEQ ID NO: 233 - protein, 5 aa, synthetic construct
  REGION 1..5: note = Synthetic: 2188-F05; CDR-H1; Kabat
DYDIH

SEQ ID NO: 234 - protein, 5 aa, synthetic construct
  REGION 1..5: note = Synthetic: 2188-D07; CDR-H1; Kabat
DFDIH

SEQ ID NO: 235 - protein, 5 aa, synthetic construct
  REGION 1..5: note = Synthetic: 2188-E02; CDR-H1; Kabat
DYEIH

SEQ ID NO: 236 - protein, 5 aa, synthetic construct
  REGION 1..5: note = Synthetic: 2188-D06; CDR-H1; Kabat
DYDIH

SEQ ID NO: 237 - protein, 5 aa, synthetic construct
  REGION 1..5: note = Synthetic: 2188-F09; CDR-H1; Kabat
DYDIH

SEQ ID NO: 238 - protein, 5 aa, synthetic construct
  REGION 1..5: note = Synthetic: 2188-F10; CDR-H1; Kabat
VFDIH

SEQ ID NO: 239 - protein, 5 aa, synthetic construct
  REGION 1..5: note = Synthetic: 2188-B05; CDR-H1; Kabat
DYDIH

SEQ ID NO: 240 - protein, 5 aa, synthetic construct
  REGION 1..5: note = Synthetic: 1943-C02; CDR-H1; Kabat
DYYIH

SEQ ID NO: 241 - protein, 5 aa, synthetic construct
  REGION 1..5: note = Synthetic: 2193-D04; CDR-H1; Kabat
GFYIH

SEQ ID NO: 242 - protein, 5 aa, synthetic construct
  REGION 1..5: note = Synthetic: 2193-E10; CDR-H1; Kabat
DYYIH

SEQ ID NO: 243 - protein, 5 aa, synthetic construct
  REGION 1..5: note = Synthetic: 2193-E06; CDR-H1; Kabat
DYYIH

SEQ ID NO: 244 - protein, 5 aa, synthetic construct
  REGION 1..5: note = Synthetic: 2193-E04; CDR-H1; Kabat
DRYIH

SEQ ID NO: 245 - protein, 5 aa, synthetic construct
  REGION 1..5: note = Synthetic: 2193-B09; CDR-H1; Kabat
NSYIH

SEQ ID NO: 246 - protein, 5 aa, synthetic construct
  REGION 1..5: note = Synthetic: 2193-D11; CDR-H1; Kabat
AYYIH

SEQ ID NO: 247 - protein, 5 aa, synthetic construct
  REGION 1..5: note = Synthetic: 2193-B02; CDR-H1; Kabat
NSYIH

SEQ ID NO: 248 - protein, 5 aa, synthetic construct
  REGION 1..5: note = Synthetic: 2193-D05; CDR-H1; Kabat
DHYIH

SEQ ID NO: 249 - protein, 5 aa, synthetic construct
  REGION 1..5: note = Synthetic: 2193-E11; CDR-H1; Kabat
DYYIH

SEQ ID NO: 250 - protein, 5 aa, synthetic construct
  REGION 1..5: note = Synthetic: 2193-D06; CDR-H1; Kabat
DYYIH

SEQ ID NO: 251 - protein, 5 aa, synthetic construct
  REGION 1..5: note = Synthetic: 2193-C02; CDR-H1; Kabat
DYYIH

SEQ ID NO: 252 - protein, 5 aa, synthetic construct
  REGION 1..5: note = Synthetic: 2193-B03; CDR-H1; Kabat
DYYIH

SEQ ID NO: 253 - protein, 5 aa, synthetic construct
  REGION 1..5: note = Synthetic: 2193-A02; CDR-H1; Kabat
ASYIH

SEQ ID NO: 254 - protein, 5 aa, synthetic construct
  REGION 1..5: note = Synthetic: 2193-E05; CDR-H1; Kabat
RSYIH

SEQ ID NO: 255 - protein, 5 aa, synthetic construct
  REGION 1..5: note = Synthetic: 2193-A06; CDR-H1; Kabat
AYYIH

SEQ ID NO: 256 - protein, 5 aa, synthetic construct
  REGION 1..5: note = Synthetic: 2193-C04; CDR-H1; Kabat
TSYIH

SEQ ID NO: 257 - protein, 5 aa, synthetic construct
  REGION 1..5: note = Synthetic: 2193-E08; CDR-H1; Kabat
EYYIH

SEQ ID NO: 258 - protein, 5 aa, synthetic construct
  REGION 1..5: note = Synthetic: 2193-B10; CDR-H1; Kabat
DYYVH

SEQ ID NO: 259 - protein, 5 aa, synthetic construct
  REGION 1..5: note = Synthetic: 2193-D08; CDR-H1; Kabat
RSYIH

SEQ ID NO: 260 - protein, 5 aa, synthetic construct
  REGION 1..5: note = Synthetic: 2193-B08; CDR-H1; Kabat
DYYIH

SEQ ID NO: 261 - protein, 5 aa, synthetic construct
  REGION 1..5: note = Synthetic: 2193-C05; CDR-H1; Kabat
HSYIH

SEQ ID NO: 262 - protein, 5 aa, synthetic construct
  REGION 1..5: note = Synthetic: 2193-D10; CDR-H1; Kabat
VSYIH

SEQ ID NO: 263 - protein, 5 aa, synthetic construct
  REGION 1..5: note = Synthetic: 2193-D03; CDR-H1; Kabat
DHYIH

SEQ ID NO: 264 - protein, 5 aa, synthetic construct
  REGION 1..5: note = Synthetic: 2193-A09; CDR-H1; Kabat
KHHIH

SEQ ID NO: 265 - protein, 5 aa, synthetic construct
  REGION 1..5: note = Synthetic: 2193-A10; CDR-H1; Kabat
DYYIH

SEQ ID NO: 266 - protein, 5 aa, synthetic construct
  REGION 1..5: note = Synthetic: 2193-E07; CDR-H1; Kabat
KVYIH

SEQ ID NO: 267 - protein, 5 aa, synthetic construct
  REGION 1..5: note = Synthetic: 2193-A07; CDR-H1; Kabat
ASDIH

SEQ ID NO: 268 - protein, 5 aa, synthetic construct
  REGION 1..5: note = Synthetic: 2193-E03; CDR-H1; Kabat
ASYIH

SEQ ID NO: 269 - protein, 5 aa, synthetic construct
  REGION 1..5: note = Synthetic: 2193-C08; CDR-H1; Kabat
HYYIH

SEQ ID NO: 270 - protein, 5 aa, synthetic construct
  REGION 1..5: note = Synthetic: 2193-B11; CDR-H1; Kabat
GSYIH

SEQ ID NO: 271 - protein, 5 aa, synthetic construct
  REGION 1..5: note = Synthetic: 2193-A03; CDR-H1; Kabat
NYHIH

SEQ ID NO: 272 - protein, 5 aa, synthetic construct
  REGION 1..5: note = Synthetic: 2193-D02; CDR-H1; Kabat
DYYIH

SEQ ID NO: 273 - protein, 5 aa, synthetic construct
  REGION 1..5: note = Synthetic: 2193-B06; CDR-H1; Kabat
GYYIH

SEQ ID NO: 274 - protein, 5 aa, synthetic construct
  REGION 1..5: note = Synthetic: 2193-B01; CDR-H1; Kabat
GYYIH

SEQ ID NO: 275 - protein, 5 aa, synthetic construct
  REGION 1..5: note = Synthetic: 2193-D09; CDR-H1; Kabat
GYYIH

SEQ ID NO: 276 - protein, 5 aa, synthetic construct
  REGION 1..5: note = Synthetic: 2193-E09; CDR-H1; Kabat
AYYIH

SEQ ID NO: 277 - protein, 5 aa, synthetic construct
  REGION 1..5: note = Synthetic: 2193-B07; CDR-H1; Kabat
NSYIH

SEQ ID NO: 278 - protein, 5 aa, synthetic construct
  REGION 1..5: note = Synthetic: 2193-C06; CDR-H1; Kabat
VHYIH

SEQ ID NO: 279 - protein, 5 aa, synthetic construct
  REGION 1..5: note = Synthetic: 2193-B05; CDR-H1; Kabat
QYYIH

SEQ ID NO: 280 - protein, 5 aa, synthetic construct
  REGION 1..5: note = Synthetic: 2193-A11; CDR-H1; Kabat
AYYIH

SEQ ID NO: 281 - protein, 5 aa, synthetic construct
  REGION 1..5: note = Synthetic: 2193-E02; CDR-H1; Kabat
DYYIH

SEQ ID NO: 282 - protein, 5 aa, synthetic construct
  REGION 1..5: note = Synthetic: 2193-A08; CDR-H1; Kabat
DSYIH

SEQ ID NO: 283 - protein, 5 aa, synthetic construct
  REGION 1..5: note = Synthetic: 2193-C11; CDR-H1; Kabat
DSYIH

SEQ ID NO: 284 - protein, 5 aa, synthetic construct
  REGION 1..5: note = Synthetic: 2193-A01; CDR-H1; Kabat
DHYIH

SEQ ID NO: 285 - protein, 5 aa, synthetic construct
  REGION 1..5: note = Synthetic: 2193-D07; CDR-H1; Kabat
DYNIH

SEQ ID NO: 286 - protein, 5 aa, synthetic construct
  REGION 1..5: note = Synthetic: 2193-A05; CDR-H1; Kabat
GYYIH

SEQ ID NO: 287 - protein, 5 aa, synthetic construct
  REGION 1..5: note = Synthetic: 2193-C03; CDR-H1; Kabat
YSYIH

SEQ ID NO: 288 - protein, 5 aa, synthetic construct
  REGION 1..5: note = Synthetic: 2193-C09; CDR-H1; Kabat
HSYIH

SEQ ID NO: 289 - protein, 5 aa, synthetic construct
  REGION 1..5: note = Synthetic: 2193-D01; CDR-H1; Kabat
DSYIH

SEQ ID NO: 290 - protein, 5 aa, synthetic construct
  REGION 1..5: note = Synthetic: 2193-E01; CDR-H1; Kabat
GKYIH

SEQ ID NO: 291 - protein, 5 aa, synthetic construct
  REGION 1..5: note = Synthetic: 2193-C07; CDR-H1; Kabat
DQDIH

SEQ ID NO: 292 - protein, 5 aa, synthetic construct
  REGION 1..5: note = Synthetic: 2193-B04; CDR-H1; Kabat
NSYIH

SEQ ID NO: 293 - protein, 5 aa, synthetic construct
  REGION 1..5: note = Synthetic: 2193-C10; CDR-H1; Kabat
NAYIH

SEQ ID NO: 294 - protein, 5 aa, synthetic construct
  REGION 1..5: note = Synthetic: 2193-C01; CDR-H1; Kabat
DHYIH

SEQ ID NO: 295 - protein, 5 aa, synthetic construct
  REGION 1..5: note = Synthetic: 2193-A04; CDR-H1; Kabat
SYYIH

SEQ ID NO: 296 - protein, 5 aa, synthetic construct
  REGION 1..5: note = Synthetic: 1944-A07; CDR-H1; Kabat
GYYIH

SEQ ID NO: 297 - protein, 5 aa, synthetic construct
  REGION 1..5: note = Synthetic: 2194-A02; CDR-H1; Kabat
GYYIH

SEQ ID NO: 298 - protein, 5 aa, synthetic construct
  REGION 1..5: note = Synthetic: 2194-A05; CDR-H1; Kabat
EYHIH

SEQ ID NO: 299 - protein, 5 aa, synthetic construct
  REGION 1..5: note = Synthetic: 2194-B04; CDR-H1; Kabat
GYHIH

SEQ ID NO: 300 - protein, 5 aa, synthetic construct
  REGION 1..5: note = Synthetic: 2194-A09; CDR-H1; Kabat
GYYIH

SEQ ID NO: 301 - protein, 5 aa, synthetic construct
  REGION 1..5: note = Synthetic: 2194-A01; CDR-H1; Kabat
GYYIH

SEQ ID NO: 302 - protein, 5 aa, synthetic construct
  REGION 1..5: note = Synthetic: 2194-B03; CDR-H1; Kabat
SYYIH

SEQ ID NO: 303 - protein, 5 aa, synthetic construct
  REGION 1..5: note = Synthetic: 2194-B02; CDR-H1; Kabat
GYYIH

SEQ ID NO: 304 - protein, 5 aa, synthetic construct
  REGION 1..5: note = Synthetic: 2194-A03; CDR-H1; Kabat
GYYIH

SEQ ID NO: 305 - protein, 5 aa, synthetic construct
  REGION 1..5: note = Synthetic: 2194-A11; CDR-H1; Kabat
QYYIH

SEQ ID NO: 306 - protein, 5 aa, synthetic construct
  REGION 1..5: note = Synthetic: 2194-A08; CDR-H1; Kabat
GYHIH

SEQ ID NO: 307 - protein, 5 aa, synthetic construct
  REGION 1..5: note = Synthetic: 2194-A04; CDR-H1; Kabat
GYYIH

SEQ ID NO: 308 - protein, 5 aa, synthetic construct
  REGION 1..5: note = Synthetic: 2194-A10; CDR-H1; Kabat
QYYIH

SEQ ID NO: 309 - protein, 5 aa, synthetic construct
  REGION 1..5: note = Synthetic: 2194-A07; CDR-H1; Kabat
DYYIH

SEQ ID NO: 310 - protein, 5 aa, synthetic construct
  REGION 1..5: note = Synthetic: 2194-B01; CDR-H1; Kabat
SYYIH

SEQ ID NO: 311 - protein, 5 aa, synthetic construct
  REGION 1..5: note = Synthetic: 2194-A06; CDR-H1; Kabat
DYYIH

SEQ ID NO: 312 - protein, 5 aa, synthetic construct
  REGION 1..5: note = Synthetic: 2196-C01; CDR-H1; Kabat
GYYIH

SEQ ID NO: 313 - protein, 5 aa, synthetic construct
  REGION 1..5: note = Synthetic: 2196-A02; CDR-H1; Kabat
GYYIH

SEQ ID NO: 314 - protein, 5 aa, synthetic construct
  REGION 1..5: note = Synthetic: 2196-B03; CDR-H1; Kabat
GYYIH

SEQ ID NO: 315 - protein, 5 aa, synthetic construct
  REGION 1..5: note = Synthetic: 2196-A05; CDR-H1; Kabat
NYYIH

SEQ ID NO: 316 - protein, 5 aa, synthetic construct
  REGION 1..5: note = Synthetic: 2196-C02; CDR-H1; Kabat
GYHIH

SEQ ID NO: 317 - protein, 5 aa, synthetic construct
  REGION 1..5: note = Synthetic: 2196-B11; CDR-H1; Kabat
GYYIH

SEQ ID NO: 318 - protein, 5 aa, synthetic construct
  REGION 1..5: note = Synthetic: 2196-B08; CDR-H1; Kabat
GYYIH

SEQ ID NO: 319 - protein, 5 aa, synthetic construct
  REGION 1..5: note = Synthetic: 2196-A04; CDR-H1; Kabat
PYYIH

SEQ ID NO: 320 - protein, 5 aa, synthetic construct
  REGION 1..5: note = Synthetic: 2196-A03; CDR-H1; Kabat
QYHIH

SEQ ID NO: 321 - protein, 5 aa, synthetic construct
  REGION 1..5: note = Synthetic: 2196-B07; CDR-H1; Kabat
GYHIH

SEQ ID NO: 322 - protein, 5 aa, synthetic construct
  REGION 1..5: note = Synthetic: 2196-A06; CDR-H1; Kabat
GYYIH

SEQ ID NO: 323 - protein, 5 aa, synthetic construct
  REGION 1..5: note = Synthetic: 2196-B05; CDR-H1; Kabat
GYYIH

SEQ ID NO: 324 - protein, 5 aa, synthetic construct
  REGION 1..5: note = Synthetic: 2196-A01; CDR-H1; Kabat
GYYIH

SEQ ID NO: 325 - protein, 5 aa, synthetic construct
  REGION 1..5: note = Synthetic: 2196-B01; CDR-H1; Kabat
GYYIH

SEQ ID NO: 326 - protein, 5 aa, synthetic construct
  REGION 1..5: note = Synthetic: 2196-A09; CDR-H1; Kabat
GYYIH

SEQ ID NO: 327 - protein, 5 aa, synthetic construct
  REGION 1..5: note = Synthetic: 2196-A08; CDR-H1; Kabat
GYYIH

SEQ ID NO: 328 - protein, 5 aa, synthetic construct
  REGION 1..5: note = Synthetic: 2196-A10; CDR-H1; Kabat
GYYIH

SEQ ID NO: 329 - protein, 5 aa, synthetic construct
  REGION 1..5: note = Synthetic: 2196-B06; CDR-H1; Kabat
GYYIH

SEQ ID NO: 330 - protein, 5 aa, synthetic construct
  REGION 1..5: note = Synthetic: 2196-B09; CDR-H1; Kabat
EYYIH

SEQ ID NO: 331 - protein, 5 aa, synthetic construct
  REGION 1..5: note = Synthetic: 2196-C03; CDR-H1; Kabat
GYYIH

SEQ ID NO: 332 - protein, 5 aa, synthetic construct
  REGION 1..5: note = Synthetic: 2196-C04; CDR-H1; Kabat
GYYIH

SEQ ID NO: 333 - protein, 5 aa, synthetic construct
  REGION 1..5: note = Synthetic: 2196-A07; CDR-H1; Kabat
GYYIH

SEQ ID NO: 334 - protein, 5 aa, synthetic construct
  REGION 1..5: note = Synthetic: 2196-A11; CDR-H1; Kabat
GYYIH

SEQ ID NO: 335 - protein, 5 aa, synthetic construct
  REGION 1..5: note = Synthetic: 2196-B02; CDR-H1; Kabat
QYYIH

SEQ ID NO: 336 - protein, 5 aa, synthetic construct
  REGION 1..5: note = Synthetic: 2196-B04; CDR-H1; Kabat
EYYIH

SEQ ID NO: 337 - protein, 5 aa, synthetic construct
  REGION 1..5: note = Synthetic: 2196-B10; CDR-H1; Kabat
GYYIH

SEQ ID NO: 338 - protein, 6 aa, synthetic construct
  REGION 1..6: note = Synthetic: 1987-C05; CDR-H2; Chothia
DPDDGS

SEQ ID NO: 339 - protein, 6 aa, synthetic construct
  REGION 1..6: note = Synthetic: 2188-D11; CDR-H2; Chothia
DPDDGS

SEQ ID NO: 340 - protein, 6 aa, synthetic construct
  REGION 1..6: note = Synthetic: 2188-B04; CDR-H2; Chothia
NPDDGD

SEQ ID NO: 341 - protein, 6 aa, synthetic construct
  REGION 1..6: note = Synthetic: 2188-C09; CDR-H2; Chothia
NPHDGD

SEQ ID NO: 342 - protein, 6 aa, synthetic construct
  REGION 1..6: note = Synthetic: 2188-G03; CDR-H2; Chothia
DPRDGS

SEQ ID NO: 343 - protein, 6 aa, synthetic construct
  REGION 1..6: note = Synthetic: 2188-E03; CDR-H2; Chothia
DPDDGS

SEQ ID NO: 344 - protein, 6 aa, synthetic construct
  REGION 1..6: note = Synthetic: 2188-B11; CDR-H2; Chothia
NPDDGD

SEQ ID NO: 345 - protein, 6 aa, synthetic construct
  REGION 1..6: note = Synthetic: 2188-E07; CDR-H2; Chothia
DPEDGF

SEQ ID NO: 346 - protein, 6 aa, synthetic construct
  REGION 1..6: note = Synthetic: 2188-B02; CDR-H2; Chothia
NPDDGD

SEQ ID NO: 347 - protein, 6 aa, synthetic construct
  REGION 1..6: note = Synthetic: 2188-C07; CDR-H2; Chothia
NPDDGD

SEQ ID NO: 348 - protein, 6 aa, synthetic construct
  REGION 1..6: note = Synthetic: 2188-A03; CDR-H2; Chothia
NPDDGD

SEQ ID NO: 349 - protein, 6 aa, synthetic construct
  REGION 1..6: note = Synthetic: 2188-F03; CDR-H2; Chothia
DPDDGS

SEQ ID NO: 350 - protein, 6 aa, synthetic construct
  REGION 1..6: note = Synthetic: 2188-D03; CDR-H2; Chothia
DPHDGS

SEQ ID NO: 351 - protein, 6 aa, synthetic construct
  REGION 1..6: note = Synthetic: 2188-C04; CDR-H2; Chothia
NPEDGD

SEQ ID NO: 352 - protein, 6 aa, synthetic construct
  REGION 1..6: note = Synthetic: 2188-D10; CDR-H2; Chothia
DPRDGA

SEQ ID NO: 353 - protein, 6 aa, synthetic construct
  REGION 1..6: note = Synthetic: 2188-A06; CDR-H2; Chothia
NPDDGD

SEQ ID NO: 354 - protein, 6 aa, synthetic construct
  REGION 1..6: note = Synthetic: 2188-C11; CDR-H2; Chothia
NPDDGD

SEQ ID NO: 355 - protein, 6 aa, synthetic construct
  REGION 1..6: note = Synthetic: 2188-F01; CDR-H2; Chothia
DPDDGW

SEQ ID NO: 356 - protein, 6 aa, synthetic construct
  REGION 1..6: note = Synthetic: 2188-E11; CDR-H2; Chothia
DPGDGA

SEQ ID NO: 357 - protein, 6 aa, synthetic construct
  REGION 1..6: note = Synthetic: 2188-A07; CDR-H2; Chothia
NPDDGD

SEQ ID NO: 358 - protein, 6 aa, synthetic construct
  REGION 1..6: note = Synthetic: 2188-C01; CDR-H2; Chothia
DPHDGS

SEQ ID NO: 359 - protein, 6 aa, synthetic construct
  REGION 1..6: note = Synthetic: 2188-F08; CDR-H2; Chothia
EPDDGA

SEQ ID NO: 360 - protein, 6 aa, synthetic construct
  REGION 1..6: note = Synthetic: 2188-E04; CDR-H2; Chothia
DPQDGS

SEQ ID NO: 361 - protein, 6 aa, synthetic construct
  REGION 1..6: note = Synthetic: 2188-B01; CDR-H2; Chothia
NPDDGD

SEQ ID NO: 362 - protein, 6 aa, synthetic construct
  REGION 1..6: note = Synthetic: 2188-F11; CDR-H2; Chothia
DPEDGA

SEQ ID NO: 363 - protein, 6 aa, synthetic construct
  REGION 1..6: note = Synthetic: 2188-B08; CDR-H2; Chothia
NPDDGD

SEQ ID NO: 364 - protein, 6 aa, synthetic construct
  REGION 1..6: note = Synthetic: 2188-C10; CDR-H2; Chothia
NPDDGD

SEQ ID NO: 365 - protein, 6 aa, synthetic construct
  REGION 1..6: note = Synthetic: 2188-C02; CDR-H2; Chothia
NPDDGD

SEQ ID NO: 366 - protein, 6 aa, synthetic construct
  REGION 1..6: note = Synthetic: 2188-B07; CDR-H2; Chothia
NPDDGD

SEQ ID NO: 367 - protein, 6 aa, synthetic construct
  REGION 1..6: note = Synthetic: 2188-A11; CDR-H2; Chothia
DPKDGA

SEQ ID NO: 368 - protein, 6 aa, synthetic construct
  REGION 1..6: note = Synthetic: 2188-D01; CDR-H2; Chothia
NPYDGD

SEQ ID NO: 369 - protein, 6 aa, synthetic construct
  REGION 1..6: note = Synthetic: 2188-E09; CDR-H2; Chothia
DPQDGW

SEQ ID NO: 370 - protein, 6 aa, synthetic construct
  REGION 1..6: note = Synthetic: 2188-E06; CDR-H2; Chothia
DPEDGA

SEQ ID NO: 371 - protein, 6 aa, synthetic construct
  REGION 1..6: note = Synthetic: 2188-B03; CDR-H2; Chothia
DPGDGA

SEQ ID NO: 372 - protein, 6 aa, synthetic construct
  REGION 1..6: note = Synthetic: 2188-F06; CDR-H2; Chothia
DPDDGA

SEQ ID NO: 373 - protein, 6 aa, synthetic construct
  REGION 1..6: note = Synthetic: 2188-D02; CDR-H2; Chothia
DPEDGA

SEQ ID NO: 374 - protein, 6 aa, synthetic construct
  REGION 1..6: note = Synthetic: 2188-B06; CDR-H2; Chothia
NPDDGD

SEQ ID NO: 375 - protein, 6 aa, synthetic construct
  REGION 1..6: note = Synthetic: 2188-D09; CDR-H2; Chothia
DPGDGS

SEQ ID NO: 376 - protein, 6 aa, synthetic construct
  REGION 1..6: note = Synthetic: 2188-F02; CDR-H2; Chothia
DPSDGS

SEQ ID NO: 377 - protein, 6 aa, synthetic construct
  REGION 1..6: note = Synthetic: 2188-E10; CDR-H2; Chothia
DPRDGA

SEQ ID NO: 378 - protein, 6 aa, synthetic construct
  REGION 1..6: note = Synthetic: 2188-A09; CDR-H2; Chothia
NPDDGD

SEQ ID NO: 379 - protein, 6 aa, synthetic construct
  REGION 1..6: note = Synthetic: 2188-D04; CDR-H2; Chothia
DPADGS

SEQ ID NO: 380 - protein, 6 aa, synthetic construct
  REGION 1..6: note = Synthetic: 2188-A05; CDR-H2; Chothia
NPDDGD

SEQ ID NO: 381 - protein, 6 aa, synthetic construct
  REGION 1..6: note = Synthetic: 2188-E01; CDR-H2; Chothia
DPQDGA

SEQ ID NO: 382 - protein, 6 aa, synthetic construct
  REGION 1..6: note = Synthetic: 2188-G01; CDR-H2; Chothia
DPNDGA

SEQ ID NO: 383 - protein, 6 aa, synthetic construct
  REGION 1..6: note = Synthetic: 2188-B09; CDR-H2; Chothia
DPKDGW

SEQ ID NO: 384 - protein, 6 aa, synthetic construct
  REGION 1..6: note = Synthetic: 2188-F07; CDR-H2; Chothia
DPNDGS

SEQ ID NO: 385 - protein, 6 aa, synthetic construct
  REGION 1..6: note = Synthetic: 2188-D08; CDR-H2; Chothia
DPDDGA

SEQ ID NO: 386 - protein, 6 aa, synthetic construct
  REGION 1..6: note = Synthetic: 2188-D05; CDR-H2; Chothia
DPHDGW

SEQ ID NO: 387 - protein, 6 aa, synthetic construct
  REGION 1..6: note = Synthetic: 2188-C03; CDR-H2; Chothia
NPDDGD

SEQ ID NO: 388 - protein, 6 aa, synthetic construct
  REGION 1..6: note = Synthetic: 2188-E08; CDR-H2; Chothia
DPQDGS

SEQ ID NO: 389 - protein, 6 aa, synthetic construct
  REGION 1..6: note = Synthetic: 2188-C06; CDR-H2; Chothia
NPDDGD

SEQ ID NO: 390 - protein, 6 aa, synthetic construct
  REGION 1..6: note = Synthetic: 2188-A08; CDR-H2; Chothia
NPFDGD

SEQ ID NO: 391 - protein, 6 aa, synthetic construct
  REGION 1..6: note = Synthetic: 2188-B10; CDR-H2; Chothia
NPHDGD

SEQ ID NO: 392 - protein, 6 aa, synthetic construct
  REGION 1..6: note = Synthetic: 2188-G02; CDR-H2; Chothia
DPNDGA

SEQ ID NO: 393 - protein, 6 aa, synthetic construct
  REGION 1..6: note = Synthetic: 2188-C05; CDR-H2; Chothia
DPRDGS

SEQ ID NO: 394 - protein, 6 aa, synthetic construct
  REGION 1..6: note = Synthetic: 2188-A10; CDR-H2; Chothia
NPEDGD

SEQ ID NO: 395 - protein, 6 aa, synthetic construct
  REGION 1..6: note = Synthetic: 2188-G04; CDR-H2; Chothia
DPRDGS

SEQ ID NO: 396 - protein, 6 aa, synthetic construct
  REGION 1..6: note = Synthetic: 2188-C08; CDR-H2; Chothia
NPDDGD

SEQ ID NO: 397 - protein, 6 aa, synthetic construct
  REGION 1..6: note = Synthetic: 2188-E05; CDR-H2; Chothia
EPWDGS

SEQ ID NO: 398 - protein, 6 aa, synthetic construct
  REGION 1..6: note = Synthetic: 2188-A02; CDR-H2; Chothia
NPEDGD

SEQ ID NO: 399 - protein, 6 aa, synthetic construct
  REGION 1..6: note = Synthetic: 2188-F04; CDR-H2; Chothia
DPDDGA

SEQ ID NO: 400 - protein, 6 aa, synthetic construct
  REGION 1..6: note = Synthetic: 2188-F05; CDR-H2; Chothia
DPRDGA

SEQ ID NO: 401 - protein, 6 aa, synthetic construct
  REGION 1..6: note = Synthetic: 2188-D07; CDR-H2; Chothia
DPTDGA

SEQ ID NO: 402 - protein, 6 aa, synthetic construct
  REGION 1..6: note = Synthetic: 2188-E02; CDR-H2; Chothia
EPHDGS

SEQ ID NO: 403 - protein, 6 aa, synthetic construct
  REGION 1..6: note = Synthetic: 2188-D06; CDR-H2; Chothia
DPNDGA

SEQ ID NO: 404 - protein, 6 aa, synthetic construct
  REGION 1..6: note = Synthetic: 2188-F09; CDR-H2; Chothia
DPNDGS

SEQ ID NO: 405 - protein, 6 aa, synthetic construct
  REGION 1..6: note = Synthetic: 2188-F10; CDR-H2; Chothia
DPNDGA

SEQ ID NO: 406 - protein, 6 aa, synthetic construct
  REGION 1..6: note = Synthetic: 2188-B05; CDR-H2; Chothia
DPDDGW

SEQ ID NO: 407 - protein, 6 aa, synthetic construct
  REGION 1..6: note = Synthetic: 1943-C02; CDR-H2; Chothia
DPNSGS

SEQ ID NO: 408 - protein, 6 aa, synthetic construct
  REGION 1..6: note = Synthetic: 2193-D04; CDR-H2; Chothia
DPNGGS

SEQ ID NO: 409 - protein, 6 aa, synthetic construct
  REGION 1..6: note = Synthetic: 2193-E10; CDR-H2; Chothia
DPVNGS

SEQ ID NO: 410 - protein, 6 aa, synthetic construct
  REGION 1..6: note = Synthetic: 2193-E06; CDR-H2; Chothia
DPKNGS

SEQ ID NO: 411 - protein, 6 aa, synthetic construct
  REGION 1..6: note = Synthetic: 2193-E04; CDR-H2; Chothia
DPSLGS

SEQ ID NO: 412 - protein, 6 aa, synthetic construct
  REGION 1..6: note = Synthetic: 2193-B09; CDR-H2; Chothia
DPNNGS

SEQ ID NO: 413 - protein, 6 aa, synthetic construct
  REGION 1..6: note = Synthetic: 2193-D11; CDR-H2; Chothia
DPNTGS

SEQ ID NO: 414 - protein, 6 aa, synthetic construct
  REGION 1..6: note = Synthetic: 2193-B02; CDR-H2; Chothia
DPTRGS

SEQ ID NO: 415 - protein, 6 aa, synthetic construct
  REGION 1..6: note = Synthetic: 2193-D05; CDR-H2; Chothia
DPNSGS

SEQ ID NO: 416 - protein, 6 aa, synthetic construct
  REGION 1..6: note = Synthetic: 2193-E11; CDR-H2; Chothia
EPNSGA

SEQ ID NO: 417 - protein, 6 aa, synthetic construct
  REGION 1..6: note = Synthetic: 2193-D06; CDR-H2; Chothia
DPHSGS

SEQ ID NO: 418 - protein, 6 aa, synthetic construct
  REGION 1..6: note = Synthetic: 2193-C02; CDR-H2; Chothia
DPQSGS

SEQ ID NO: 419 - protein, 6 aa, synthetic construct
  REGION 1..6: note = Synthetic: 2193-B03; CDR-H2; Chothia
DPVSGS

SEQ ID NO: 420 - protein, 6 aa, synthetic construct
  REGION 1..6: note = Synthetic: 2193-A02; CDR-H2; Chothia
DPKSGS

SEQ ID NO: 421 - protein, 6 aa, synthetic construct
  REGION 1..6: note = Synthetic: 2193-E05; CDR-H2; Chothia
DPNGSS

SEQ ID NO: 422 - protein, 6 aa, synthetic construct
  REGION 1..6: note = Synthetic: 2193-A06; CDR-H2; Chothia
DPDSGS

SEQ ID NO: 423 - protein, 6 aa, synthetic construct
  REGION 1..6: note = Synthetic: 2193-C04; CDR-H2; Chothia
DPKSGS

SEQ ID NO: 424 - protein, 6 aa, synthetic construct
  REGION 1..6: note = Synthetic: 2193-E08; CDR-H2; Chothia
DPHSGS

SEQ ID NO: 425 - protein, 6 aa, synthetic construct
  REGION 1..6: note = Synthetic: 2193-B10; CDR-H2; Chothia
DPNSGY

SEQ ID NO: 426 - protein, 6 aa, synthetic construct
  REGION 1..6: note = Synthetic: 2193-D08; CDR-H2; Chothia
DPSGGS

SEQ ID NO: 427 - protein, 6 aa, synthetic construct
  REGION 1..6: note = Synthetic: 2193-B08; CDR-H2; Chothia
DPSPGA

SEQ ID NO: 428 - protein, 6 aa, synthetic construct
  REGION 1..6: note = Synthetic: 2193-C05; CDR-H2; Chothia
DPHNGS

SEQ ID NO: 429 - protein, 6 aa, synthetic construct
  REGION 1..6: note = Synthetic: 2193-D10; CDR-H2; Chothia
DPNSGF

SEQ ID NO: 430 - protein, 6 aa, synthetic construct
  REGION 1..6: note = Synthetic: 2193-D03; CDR-H2; Chothia
DPNTGS

SEQ ID NO: 431 - protein, 6 aa, synthetic construct
  REGION 1..6: note = Synthetic: 2193-A09; CDR-H2; Chothia
DPKGGS

SEQ ID NO: 432 - protein, 6 aa, synthetic construct
  REGION 1..6: note = Synthetic: 2193-A10; CDR-H2; Chothia
DPKSGY

SEQ ID NO: 433 - protein, 6 aa, synthetic construct
  REGION 1..6: note = Synthetic: 2193-E07; CDR-H2; Chothia
DPTIGS

SEQ ID NO: 434 - protein, 6 aa, synthetic construct
  REGION 1..6: note = Synthetic: 2193-A07; CDR-H2; Chothia
DPNTGT

SEQ ID NO: 435 - protein, 6 aa, synthetic construct
  REGION 1..6: note = Synthetic: 2193-E03; CDR-H2; Chothia
DPKGGS

SEQ ID NO: 436 - protein, 6 aa, synthetic construct
  REGION 1..6: note = Synthetic: 2193-C08; CDR-H2; Chothia
DPYPGS

SEQ ID NO: 437 - protein, 6 aa, synthetic construct
  REGION 1..6: note = Synthetic: 2193-B11; CDR-H2; Chothia
DPKSGF

SEQ ID NO: 438 - protein, 6 aa, synthetic construct
  REGION 1..6: note = Synthetic: 2193-A03; CDR-H2; Chothia
DPKSGS

SEQ ID NO: 439 - protein, 6 aa, synthetic construct
  REGION 1..6: note = Synthetic: 2193-D02; CDR-H2; Chothia
DPESGS

SEQ ID NO: 440 - protein, 6 aa, synthetic construct
  REGION 1..6: note = Synthetic: 2193-B06; CDR-H2; Chothia
DPHPGS

SEQ ID NO: 441 - protein, 6 aa, synthetic construct
  REGION 1..6: note = Synthetic: 2193-B01; CDR-H2; Chothia
DPRSGY

SEQ ID NO: 442 - protein, 6 aa, synthetic construct
  REGION 1..6: note = Synthetic: 2193-D09; CDR-H2; Chothia
DPNSGS

SEQ ID NO: 443 - protein, 6 aa, synthetic construct
  REGION 1..6: note = Synthetic: 2193-E09; CDR-H2; Chothia
DPGSGY

SEQ ID NO: 444 - protein, 6 aa, synthetic construct
  REGION 1..6: note = Synthetic: 2193-B07; CDR-H2; Chothia
DPNSGS

SEQ ID NO: 445 - protein, 6 aa, synthetic construct
  REGION 1..6: note = Synthetic: 2193-C06; CDR-H2; Chothia
DPISGS

SEQ ID NO: 446 - protein, 6 aa, synthetic construct
  REGION 1..6: note = Synthetic: 2193-B05; CDR-H2; Chothia
DPIGGS

SEQ ID NO: 447 - protein, 6 aa, synthetic construct
  REGION 1..6: note = Synthetic: 2193-A11; CDR-H2; Chothia
DPKSGS

SEQ ID NO: 448 - protein, 6 aa, synthetic construct
  REGION 1..6: note = Synthetic: 2193-E02; CDR-H2; Chothia
DPTSGS

SEQ ID NO: 449 - protein, 6 aa, synthetic construct
  REGION 1..6: note = Synthetic: 2193-A08; CDR-H2; Chothia
DPNAGS

SEQ ID NO: 450 - protein, 6 aa, synthetic construct
  REGION 1..6: note = Synthetic: 2193-C11; CDR-H2; Chothia
DPKSGS

SEQ ID NO: 451 - protein, 6 aa, synthetic construct
  REGION 1..6: note = Synthetic: 2193-A01; CDR-H2; Chothia
DPTSGS

SEQ ID NO: 452 - protein, 6 aa, synthetic construct
  REGION 1..6: note = Synthetic: 2193-D07; CDR-H2; Chothia
GPADGS

SEQ ID NO: 453 - protein, 6 aa, synthetic construct
  REGION 1..6: note = Synthetic: 2193-A05; CDR-H2; Chothia
DPNSGS

SEQ ID NO: 454 - protein, 6 aa, synthetic construct
  REGION 1..6: note = Synthetic: 2193-C03; CDR-H2; Chothia
EPKSGS

SEQ ID NO: 455 - protein, 6 aa, synthetic construct
  REGION 1..6: note = Synthetic: 2193-C09; CDR-H2; Chothia
DPISGS

SEQ ID NO: 456 - protein, 6 aa, synthetic construct
  REGION 1..6: note = Synthetic: 2193-D01; CDR-H2; Chothia
DPTSGP

SEQ ID NO: 457 - protein, 6 aa, synthetic construct
  REGION 1..6: note = Synthetic: 2193-E01; CDR-H2; Chothia
DPKSGS

SEQ ID NO: 458 - protein, 6 aa, synthetic construct
  REGION 1..6: note = Synthetic: 2193-C07; CDR-H2; Chothia
DPTRGA

SEQ ID NO: 459 - protein, 6 aa, synthetic construct
  REGION 1..6: note = Synthetic: 2193-B04; CDR-H2; Chothia
EPKNGS

SEQ ID NO: 460 - protein, 6 aa, synthetic construct
  REGION 1..6: note = Synthetic: 2193-C10; CDR-H2; Chothia
DPRSGS

SEQ ID NO: 461 - protein, 6 aa, synthetic construct
  REGION 1..6: note = Synthetic: 2193-C01; CDR-H2; Chothia
DPKSGS

SEQ ID NO: 462 - protein, 6 aa, synthetic construct
  REGION 1..6: note = Synthetic: 2193-A04; CDR-H2; Chothia
DPNSGS

SEQ ID NO: 463 - protein, 6 aa, synthetic construct
  REGION 1..6: note = Synthetic: 1944-A07; CDR-H2; Chothia
SPNSGS

SEQ ID NO: 464 - protein, 6 aa, synthetic construct
  REGION 1..6: note = Synthetic: 2194-A02; CDR-H2; Chothia
SPLAGN

SEQ ID NO: 465 - protein, 6 aa, synthetic construct
  REGION 1..6: note = Synthetic: 2194-A05; CDR-H2; Chothia
SPNSGS

SEQ ID NO: 466 - protein, 6 aa, synthetic construct
  REGION 1..6: note = Synthetic: 2194-B04; CDR-H2; Chothia
SPLAGN

SEQ ID NO: 467 - protein, 6 aa, synthetic construct
  REGION 1..6: note = Synthetic: 2194-A09; CDR-H2; Chothia
TPLSGA

SEQ ID NO: 468 - protein, 6 aa, synthetic construct
  REGION 1..6: note = Synthetic: 2194-A01; CDR-H2; Chothia
SPLAGN

SEQ ID NO: 469 - protein, 6 aa, synthetic construct
  REGION 1..6: note = Synthetic: 2194-B03; CDR-H2; Chothia
SPLAGN

SEQ ID NO: 470 - protein, 6 aa, synthetic construct
  REGION 1..6: note = Synthetic: 2194-B02; CDR-H2; Chothia
SPLAGN

SEQ ID NO: 471 - protein, 6 aa, synthetic construct
  REGION 1..6: note = Synthetic: 2194-A03; CDR-H2; Chothia
TPNSGT

SEQ ID NO: 472 - protein, 6 aa, synthetic construct
  REGION 1..6: note = Synthetic: 2194-A11; CDR-H2; Chothia
SPNSGY

SEQ ID NO: 473 - protein, 6 aa, synthetic construct
  REGION 1..6: note = Synthetic: 2194-A08; CDR-H2; Chothia
APSSGY

SEQ ID NO: 474 - protein, 6 aa, synthetic construct
  REGION 1..6: note = Synthetic: 2194-A04; CDR-H2; Chothia
SPLAGN

SEQ ID NO: 475 - protein, 6 aa, synthetic construct
  REGION 1..6: note = Synthetic: 2194-A10; CDR-H2; Chothia
SPNSGS

SEQ ID NO: 476 - protein, 6 aa, synthetic construct
  REGION 1..6: note = Synthetic: 2194-A07; CDR-H2; Chothia
SPLAGN

SEQ ID NO: 477 - protein, 6 aa, synthetic construct
  REGION 1..6: note = Synthetic: 2194-B01; CDR-H2; Chothia
SPLAGN

SEQ ID NO: 478 - protein, 6 aa, synthetic construct
  REGION 1..6: note = Synthetic: 2194-A06; CDR-H2; Chothia
SPLAGN

SEQ ID NO: 479 - protein, 6 aa, synthetic construct
  REGION 1..6: note = Synthetic: 2196-C01; CDR-H2; Chothia
TPLSGA

SEQ ID NO: 480 - protein, 6 aa, synthetic construct
  REGION 1..6: note = Synthetic: 2196-A02; CDR-H2; Chothia
SPNSGA

SEQ ID NO: 481 - protein, 6 aa, synthetic construct
  REGION 1..6: note = Synthetic: 2196-B03; CDR-H2; Chothia
SPNSGA

SEQ ID NO: 482 - protein, 6 aa, synthetic construct
  REGION 1..6: note = Synthetic: 2196-A05; CDR-H2; Chothia
SPNSGA

SEQ ID NO: 483 - protein, 6 aa, synthetic construct
  REGION 1..6: note = Synthetic: 2196-C02; CDR-H2; Chothia
SPNSGW

SEQ ID NO: 484 - protein, 6 aa, synthetic construct
  REGION 1..6: note = Synthetic: 2196-B11; CDR-H2; Chothia
SPNSGY

SEQ ID NO: 485 - protein, 6 aa, synthetic construct
  REGION 1..6: note = Synthetic: 2196-B08; CDR-H2; Chothia
SPNSGY

SEQ ID NO: 486 - protein, 6 aa, synthetic construct
  REGION 1..6: note = Synthetic: 2196-A04; CDR-H2; Chothia
YPISGH

SEQ ID NO: 487 - protein, 6 aa, synthetic construct
  REGION 1..6: note = Synthetic: 2196-A03; CDR-H2; Chothia
SPNSGS

SEQ ID NO: 488 - protein, 6 aa, synthetic construct
  REGION 1..6: note = Synthetic: 2196-B07; CDR-H2; Chothia
SPNSGA

SEQ ID NO: 489 - protein, 6 aa, synthetic construct
  REGION 1..6: note = Synthetic: 2196-A06; CDR-H2; Chothia
SPNSGS

SEQ ID NO: 490 - protein, 6 aa, synthetic construct
  REGION 1..6: note = Synthetic: 2196-B05; CDR-H2; Chothia
SPNSGA

SEQ ID NO: 491 - protein, 6 aa, synthetic construct
  REGION 1..6: note = Synthetic: 2196-A01; CDR-H2; Chothia
SPNSGY

SEQ ID NO: 492 - protein, 6 aa, synthetic construct
  REGION 1..6: note = Synthetic: 2196-B01; CDR-H2; Chothia
SPNSGY

SEQ ID NO: 493 - protein, 6 aa, synthetic construct
  REGION 1..6: note = Synthetic: 2196-A09; CDR-H2; Chothia
SPNSGA

SEQ ID NO: 494 - protein, 6 aa, synthetic construct
  REGION 1..6: note = Synthetic: 2196-A08; CDR-H2; Chothia
SPNSGS

SEQ ID NO: 495 - protein, 6 aa, synthetic construct
  REGION 1..6: note = Synthetic: 2196-A10; CDR-H2; Chothia
SPNSGA

SEQ ID NO: 496 - protein, 6 aa, synthetic construct
  REGION 1..6: note = Synthetic: 2196-B06; CDR-H2; Chothia
SPNSGS

SEQ ID NO: 497 - protein, 6 aa, synthetic construct
  REGION 1..6: note = Synthetic: 2196-B09; CDR-H2; Chothia
SPNSGS

SEQ ID NO: 498 - protein, 6 aa, synthetic construct
  REGION 1..6: note = Synthetic: 2196-C03; CDR-H2; Chothia
SPNSGS

SEQ ID NO: 499 - protein, 6 aa, synthetic construct
  REGION 1..6: note = Synthetic: 2196-C04; CDR-H2; Chothia
APLSGS

SEQ ID NO: 500 - protein, 6 aa, synthetic construct
  REGION 1..6: note = Synthetic: 2196-A07; CDR-H2; Chothia
SPNSGS

SEQ ID NO: 501 - protein, 6 aa, synthetic construct
  REGION 1..6: note = Synthetic: 2196-A11; CDR-H2; Chothia
SPNSGT

SEQ ID NO: 502 - protein, 6 aa, synthetic construct
  REGION 1..6: note = Synthetic: 2196-B02; CDR-H2; Chothia
SPNSGQ

SEQ ID NO: 503 - protein, 6 aa, synthetic construct
  REGION 1..6: note = Synthetic: 2196-B04; CDR-H2; Chothia
SPNSGA

SEQ ID NO: 504 - protein, 6 aa, synthetic construct
  REGION 1..6: note = Synthetic: 2196-B10; CDR-H2; Chothia
TPLSGA

SEQ ID NO: 505 - protein, 17 aa, synthetic construct
  REGION 1..17: note = Synthetic: 1987-C05; CDR-H2; Kabat
GIDPDDGSTDYADSVKG

SEQ ID NO: 506 - protein, 17 aa, synthetic construct
  REGION 1..17: note = Synthetic: 2188-D11; CDR-H2; Kabat
LIDPDDGSTDEADSVKG

SEQ ID NO: 507 - protein, 17 aa, synthetic construct
  REGION 1..17: note = Synthetic: 2188-B04; CDR-H2; Kabat
WINPDDGDTYYADSVKG

SEQ ID NO: 508 - protein, 17 aa, synthetic construct
  REGION 1..17: note = Synthetic: 2188-C09; CDR-H2; Kabat
WINPHDGDTYYADSVKG

SEQ ID NO: 509 - protein, 17 aa, synthetic construct
  REGION 1..17: note = Synthetic: 2188-G03; CDR-H2; Kabat
GIDPRDGSTDYADSVKG

SEQ ID NO: 510 - protein, 17 aa, synthetic construct
  REGION 1..17: note = Synthetic: 2188-E03; CDR-H2; Kabat
GIDPDDGSTDYADSVKG

SEQ ID NO: 511 - protein, 17 aa, synthetic construct
  REGION 1..17: note = Synthetic: 2188-B11; CDR-H2; Kabat
WINPDDGDTFLADSVKG

SEQ ID NO: 512 - protein, 17 aa, synthetic construct
  REGION 1..17: note = Synthetic: 2188-E07; CDR-H2; Kabat
GIDPEDGFTVHADSVKG

SEQ ID NO: 513 - protein, 17 aa, synthetic construct
  REGION 1..17: note = Synthetic: 2188-B02; CDR-H2; Kabat
WINPDDGDTYLADSVKG

SEQ ID NO: 514 - protein, 17 aa, synthetic construct
  REGION 1..17: note = Synthetic: 2188-C07; CDR-H2; Kabat
WINPDDGDTYYADSVKG

SEQ ID NO: 515 - protein, 17 aa, synthetic construct
  REGION 1..17: note = Synthetic: 2188-A03; CDR-H2; Kabat
WINPDDGDTYYADSVKG

SEQ ID NO: 516 - protein, 17 aa, synthetic construct
  REGION 1..17: note = Synthetic: 2188-F03; CDR-H2; Kabat
GIDPDDGSTDYADSVKG

SEQ ID NO: 517 - protein, 17 aa, synthetic construct
  REGION 1..17: note = Synthetic: 2188-D03; CDR-H2; Kabat
GIDPHDGSTDYADSVKG

SEQ ID NO: 518 - protein, 17 aa, synthetic construct
  REGION 1..17: note = Synthetic: 2188-C04; CDR-H2; Kabat
WINPEDGDTYHADSVKG

SEQ ID NO: 519 - protein, 17 aa, synthetic construct
  REGION 1..17: note = Synthetic: 2188-D10; CDR-H2; Kabat
RIDPRDGATDYADSVKG

SEQ ID NO: 520 - protein, 17 aa, synthetic construct
  REGION 1..17: note = Synthetic: 2188-A06; CDR-H2; Kabat
WINPDDGDTYHADSVKG

SEQ ID NO: 521 - protein, 17 aa, synthetic construct
  REGION 1..17: note = Synthetic: 2188-C11; CDR-H2; Kabat
WINPDDGDTYLADSVKG

SEQ ID NO: 522 - protein, 17 aa, synthetic construct
  REGION 1..17: note = Synthetic: 2188-F01; CDR-H2; Kabat
LIDPDDGWTVSADSVKG

SEQ ID NO: 523 - protein, 17 aa, synthetic construct
  REGION 1..17: note = Synthetic: 2188-E11; CDR-H2; Kabat
GIDPGDGATDHADSVKG

SEQ ID NO: 524 - protein, 17 aa, synthetic construct
  REGION 1..17: note = Synthetic: 2188-A07; CDR-H2; Kabat
WINPDDGDTYYADSVKG

SEQ ID NO: 525 - protein, 17 aa, synthetic construct
  REGION 1..17: note = Synthetic: 2188-C01; CDR-H2; Kabat
LIDPHDGSTDSADSVKG

SEQ ID NO: 526 - protein, 17 aa, synthetic construct
  REGION 1..17: note = Synthetic: 2188-F08; CDR-H2; Kabat
RIEPDDGATDYADSVKG

SEQ ID NO: 527 - protein, 17 aa, synthetic construct
  REGION 1..17: note = Synthetic: 2188-E04; CDR-H2; Kabat
GIDPQDGSTDYADSVKG

SEQ ID NO: 528 - protein, 17 aa, synthetic construct
  REGION 1..17: note = Synthetic: 2188-B01; CDR-H2; Kabat
WINPDDGDTYYADSVKG

SEQ ID NO: 529 - protein, 17 aa, synthetic construct
  REGION 1..17: note = Synthetic: 2188-F11; CDR-H2; Kabat
GIDPEDGATDYADSVKG

SEQ ID NO: 530 - protein, 17 aa, synthetic construct
  REGION 1..17: note = Synthetic: 2188-B08; CDR-H2; Kabat
WINPDDGDTFYADSVKG

SEQ ID NO: 531 - protein, 17 aa, synthetic construct
  REGION 1..17: note = Synthetic: 2188-C10; CDR-H2; Kabat
WINPDDGDTYHADSVKG

SEQ ID NO: 532 - protein, 17 aa, synthetic construct
  REGION 1..17: note = Synthetic: 2188-C02; CDR-H2; Kabat
WINPDDGDTYYADSVKG

SEQ ID NO: 533 - protein, 17 aa, synthetic construct
  REGION 1..17: note = Synthetic: 2188-B07; CDR-H2; Kabat
WINPDDGDTYYADSVKG

SEQ ID NO: 534 - protein, 17 aa, synthetic construct
  REGION 1..17: note = Synthetic: 2188-A11; CDR-H2; Kabat
LIDPKDGATDSADSVKG

SEQ ID NO: 535 - protein, 17 aa, synthetic construct
  REGION 1..17: note = Synthetic: 2188-D01; CDR-H2; Kabat
WINPYDGDTYYADSVKG

SEQ ID NO: 536 - protein, 17 aa, synthetic construct
  REGION 1..17: note = Synthetic: 2188-E09; CDR-H2; Kabat
EIDPQDGWTVHADSVKG

SEQ ID NO: 537 - protein, 17 aa, synthetic construct
  REGION 1..17: note = Synthetic: 2188-E06; CDR-H2; Kabat
GIDPEDGATDIADSVKG

SEQ ID NO: 538 - protein, 17 aa, synthetic construct
  REGION 1..17: note = Synthetic: 2188-B03; CDR-H2; Kabat
AIDPGDGATDYADSVKG

SEQ ID NO: 539 - protein, 17 aa, synthetic construct
  REGION 1..17: note = Synthetic: 2188-F06; CDR-H2; Kabat
GIDPDDGATDYADSVKG

SEQ ID NO: 540 - protein, 17 aa, synthetic construct
  REGION 1..17: note = Synthetic: 2188-D02; CDR-H2; Kabat
GIDPEDGATDYADSVKG

SEQ ID NO: 541 - protein, 17 aa, synthetic construct
  REGION 1..17: note = Synthetic: 2188-B06; CDR-H2; Kabat
WINPDDGDTYHADSVKG

SEQ ID NO: 542 - protein, 17 aa, synthetic construct
  REGION 1..17: note = Synthetic: 2188-D09; CDR-H2; Kabat
GIDPGDGSTDYADSVKG

SEQ ID NO: 543 - protein, 17 aa, synthetic construct
  REGION 1..17: note = Synthetic: 2188-F02; CDR-H2; Kabat
WIDPSDGSTEHADSVKG

SEQ ID NO: 544 - protein, 17 aa, synthetic construct
  REGION 1..17: note = Synthetic: 2188-E10; CDR-H2; Kabat
GIDPRDGATDYADSVKG

SEQ ID NO: 545 - protein, 17 aa, synthetic construct
  REGION 1..17: note = Synthetic: 2188-A09; CDR-H2; Kabat
WINPDDGDTYYADSVKG

SEQ ID NO: 546 - protein, 17 aa, synthetic construct
  REGION 1..17: note = Synthetic: 2188-D04; CDR-H2; Kabat
GIDPADGSTDYADSVKG

SEQ ID NO: 547 - protein, 17 aa, synthetic construct
  REGION 1..17: note = Synthetic: 2188-A05; CDR-H2; Kabat
WINPDDGDTYYADSVKG

SEQ ID NO: 548 - protein, 17 aa, synthetic construct
  REGION 1..17: note = Synthetic: 2188-E01; CDR-H2; Kabat
GIDPQDGATDYADSVKG

SEQ ID NO: 549 - protein, 17 aa, synthetic construct
  REGION 1..17: note = Synthetic: 2188-G01; CDR-H2; Kabat
GIDPNDGATDYADSVKG

SEQ ID NO: 550 - protein, 17 aa, synthetic construct
  REGION 1..17: note = Synthetic: 2188-B09; CDR-H2; Kabat
VIDPKDGWTDHADSVKG

SEQ ID NO: 551 - protein, 17 aa, synthetic construct
  REGION 1..17: note = Synthetic: 2188-F07; CDR-H2; Kabat
GIDPNDGSTDHADSVKG

SEQ ID NO: 552 - protein, 17 aa, synthetic construct
  REGION 1..17: note = Synthetic: 2188-D08; CDR-H2; Kabat
GIDPDDGATDEADSVKG

SEQ ID NO: 553 - protein, 17 aa, synthetic construct
  REGION 1..17: note = Synthetic: 2188-D05; CDR-H2; Kabat
GIDPHDGWTDHADSVKG

SEQ ID NO: 554 - protein, 17 aa, synthetic construct
  REGION 1..17: note = Synthetic: 2188-C03; CDR-H2; Kabat
WINPDDGDTYYADSVKG

SEQ ID NO: 555 - protein, 17 aa, synthetic construct
  REGION 1..17: note = Synthetic: 2188-E08; CDR-H2; Kabat
GIDPQDGSTDLADSVKG

SEQ ID NO: 556 - protein, 17 aa, synthetic construct
  REGION 1..17: note = Synthetic: 2188-C06; CDR-H2; Kabat
WINPDDGDTYLADSVKG

SEQ ID NO: 557 - protein, 17 aa, synthetic construct
  REGION 1..17: note = Synthetic: 2188-A08; CDR-H2; Kabat
WINPFDGDTYYADSVKG

SEQ ID NO: 558 - protein, 17 aa, synthetic construct
  REGION 1..17: note = Synthetic: 2188-B10; CDR-H2; Kabat
WINPHDGDTYHADSVKG

SEQ ID NO: 559 - protein, 17 aa, synthetic construct
  REGION 1..17: note = Synthetic: 2188-G02; CDR-H2; Kabat
GIDPNDGATDYADSVKG

SEQ ID NO: 560 - protein, 17 aa, synthetic construct
  REGION 1..17: note = Synthetic: 2188-C05; CDR-H2; Kabat
GIDPRDGSTDSADSVKG

SEQ ID NO: 561 - protein, 17 aa, synthetic construct
  REGION 1..17: note = Synthetic: 2188-A10; CDR-H2; Kabat
WINPEDGDTSHADSVKG

SEQ ID NO: 562 - protein, 17 aa, synthetic construct
  REGION 1..17: note = Synthetic: 2188-G04; CDR-H2; Kabat
GIDPRDGSTDHADSVKG

SEQ ID NO: 563 - protein, 17 aa, synthetic construct
  REGION 1..17: note = Synthetic: 2188-C08; CDR-H2; Kabat
WINPDDGDTYHADSVKG

SEQ ID NO: 564 - protein, 17 aa, synthetic construct
  REGION 1..17: note = Synthetic: 2188-E05; CDR-H2; Kabat
GIEPWDGSTDHADSVKG

SEQ ID NO: 565 - protein, 17 aa, synthetic construct
  REGION 1..17: note = Synthetic: 2188-A02; CDR-H2; Kabat
WINPEDGDTYYADSVKG

SEQ ID NO: 566 - protein, 17 aa, synthetic construct
  REGION 1..17: note = Synthetic: 2188-F04; CDR-H2; Kabat
GIDPDDGATDYADSVKG

SEQ ID NO: 567 - protein, 17 aa, synthetic construct
  REGION 1..17: note = Synthetic: 2188-F05; CDR-H2; Kabat
GIDPRDGATDSADSVKG

SEQ ID NO: 568 - protein, 17 aa, synthetic construct
  REGION 1..17: note = Synthetic: 2188-D07; CDR-H2; Kabat
GIDPTDGATDYADSVKG

SEQ ID NO: 569 - protein, 17 aa, synthetic construct
  REGION 1..17: note = Synthetic: 2188-E02; CDR-H2; Kabat
GIEPHDGSTDYADSVKG

SEQ ID NO: 570 - protein, 17 aa, synthetic construct
  REGION 1..17: note = Synthetic: 2188-D06; CDR-H2; Kabat
GIDPNDGATDYADSVKG

SEQ ID NO: 571 - protein, 17 aa, synthetic construct
  REGION 1..17: note = Synthetic: 2188-F09; CDR-H2; Kabat
AIDPNDGSTDYADSVKG

SEQ ID NO: 572 - protein, 17 aa, synthetic construct
  REGION 1..17: note = Synthetic: 2188-F10; CDR-H2; Kabat
GIDPNDGATDHADSVKG

SEQ ID NO: 573 - protein, 17 aa, synthetic construct
  REGION 1..17: note = Synthetic: 2188-B05; CDR-H2; Kabat
VIDPDDGWTHYADSVKG

SEQ ID NO: 574 - protein, 17 aa, synthetic construct
  REGION 1..17: note = Synthetic: 1943-C02; CDR-H2; Kabat
AIDPNSGSTDYADSVKG

SEQ ID NO: 575 - protein, 17 aa, synthetic construct
  REGION 1..17: note = Synthetic: 2193-D04; CDR-H2; Kabat
AIDPNGGSTVYADSVKG

SEQ ID NO: 576 - protein, 17 aa, synthetic construct
  REGION 1..17: note = Synthetic: 2193-E10; CDR-H2; Kabat
AIDPVNGSTNNADSVKG

SEQ ID NO: 577 - protein, 17 aa, synthetic construct
  REGION 1..17: note = Synthetic: 2193-E06; CDR-H2; Kabat
VIDPKNGSTVYADSVKG

SEQ ID NO: 578 - protein, 17 aa, synthetic construct
  REGION 1..17: note = Synthetic: 2193-E04; CDR-H2; Kabat
VIDPSLGSTIDADSVKG

SEQ ID NO: 579 - protein, 17 aa, synthetic construct
  REGION 1..17: note = Synthetic: 2193-B09; CDR-H2; Kabat
IIDPNNGSTAYADSVKG

SEQ ID NO: 580 - protein, 17 aa, synthetic construct
  REGION 1..17: note = Synthetic: 2193-D11; CDR-H2; Kabat
AIDPNTGSTVDADSVKG

SEQ ID NO: 581 - protein, 17 aa, synthetic construct
  REGION 1..17: note = Synthetic: 2193-B02; CDR-H2; Kabat
TIDPTRGSTVHADSVKG

SEQ ID NO: 582 - protein, 17 aa, synthetic construct
  REGION 1..17: note = Synthetic: 2193-D05; CDR-H2; Kabat
AIDPNSGSTVYADSVKG

SEQ ID NO: 583 - protein, 17 aa, synthetic construct
  REGION 1..17: note = Synthetic: 2193-E11; CDR-H2; Kabat
GIEPNSGATVFADSVKG

SEQ ID NO: 584 - protein, 17 aa, synthetic construct
  REGION 1..17: note = Synthetic: 2193-D06; CDR-H2; Kabat
AIDPHSGSTVYADSVKG

SEQ ID NO: 585 - protein, 17 aa, synthetic construct
  REGION 1..17: note = Synthetic: 2193-C02; CDR-H2; Kabat
TIDPQSGSTVYADSVKG

SEQ ID NO: 586 - protein, 17 aa, synthetic construct
  REGION 1..17: note = Synthetic: 2193-B03; CDR-H2; Kabat
AIDPVSGSTLFADSVKG

SEQ ID NO: 587 - protein, 17 aa, synthetic construct
  REGION 1..17: note = Synthetic: 2193-A02; CDR-H2; Kabat
AIDPKSGSTYYADSVKG

SEQ ID NO: 588 - protein, 17 aa, synthetic construct
  REGION 1..17: note = Synthetic: 2193-E05; CDR-H2; Kabat
SIDPNGSSTHYADSVKG

SEQ ID NO: 589 - protein, 17 aa, synthetic construct
  REGION 1..17: note = Synthetic: 2193-A06; CDR-H2; Kabat
AIDPDSGSTHNADSVKG

SEQ ID NO: 590 - protein, 17 aa, synthetic construct
  REGION 1..17: note = Synthetic: 2193-C04; CDR-H2; Kabat
AIDPKSGSTNFADSVKG

SEQ ID NO: 591 - protein, 17 aa, synthetic construct
  REGION 1..17: note = Synthetic: 2193-E08; CDR-H2; Kabat
AIDPHSGSTNFADSVKG

SEQ ID NO: 592 - protein, 17 aa, synthetic construct
  REGION 1..17: note = Synthetic: 2193-B10; CDR-H2; Kabat
AIDPNSGYTVKADSVKG

SEQ ID NO: 593 - protein, 17 aa, synthetic construct
  REGION 1..17: note = Synthetic: 2193-D08; CDR-H2; Kabat
AIDPSGGSTNHADSVKG

SEQ ID NO: 594 - protein, 17 aa, synthetic construct
  REGION 1..17: note = Synthetic: 2193-B08; CDR-H2; Kabat
AIDPSPGATLDADSVKG

SEQ ID NO: 595 - protein, 17 aa, synthetic construct
  REGION 1..17: note = Synthetic: 2193-C05; CDR-H2; Kabat
AIDPHNGSTASADSVKG

SEQ ID NO: 596 - protein, 17 aa, synthetic construct
  REGION 1..17: note = Synthetic: 2193-D10; CDR-H2; Kabat
TIDPNSGFTVHADSVKG

SEQ ID NO: 597 - protein, 17 aa, synthetic construct
  REGION 1..17: note = Synthetic: 2193-D03; CDR-H2; Kabat
AIDPNTGSTVNADSVKG

SEQ ID NO: 598 - protein, 17 aa, synthetic construct
  REGION 1..17: note = Synthetic: 2193-A09; CDR-H2; Kabat
VIDPKGGSTVYADSVKG

SEQ ID NO: 599 - protein, 17 aa, synthetic construct
  REGION 1..17: note = Synthetic: 2193-A10; CDR-H2; Kabat
AIDPKSGYTVYADSVKG

SEQ ID NO: 600 - protein, 17 aa, synthetic construct
  REGION 1..17: note = Synthetic: 2193-E07; CDR-H2; Kabat
SIDPTIGSTHFADSVKG

SEQ ID NO: 601 - protein, 17 aa, synthetic construct
  REGION 1..17: note = Synthetic: 2193-A07; CDR-H2; Kabat
AIDPNTGTTNYADSVKG

SEQ ID NO: 602 - protein, 17 aa, synthetic construct
  REGION 1..17: note = Synthetic: 2193-E03; CDR-H2; Kabat
AIDPKGGSTRFADSVKG

SEQ ID NO: 603 - protein, 17 aa, synthetic construct
  REGION 1..17: note = Synthetic: 2193-C08; CDR-H2; Kabat
AIDPYPGSTYNADSVKG

SEQ ID NO: 604 - protein, 17 aa, synthetic construct
  REGION 1..17: note = Synthetic: 2193-B11; CDR-H2; Kabat
AIDPKSGFTSYADSVKG

SEQ ID NO: 605 - protein, 17 aa, synthetic construct
  REGION 1..17: note = Synthetic: 2193-A03; CDR-H2; Kabat
AIDPKSGSTVHADSVKG

SEQ ID NO: 606 - protein, 17 aa, synthetic construct
  REGION 1..17: note = Synthetic: 2193-D02; CDR-H2; Kabat
AIDPESGSTVYADSVKG

SEQ ID NO: 607 - protein, 17 aa, synthetic construct
  REGION 1..17: note = Synthetic: 2193-B06; CDR-H2; Kabat
AIDPHPGSTVYADSVKG

SEQ ID NO: 608 - protein, 17 aa, synthetic construct
  REGION 1..17: note = Synthetic: 2193-B01; CDR-H2; Kabat
AIDPRSGYTVYADSVKG

SEQ ID NO: 609 - protein, 17 aa, synthetic construct
  REGION 1..17: note = Synthetic: 2193-D09; CDR-H2; Kabat
AIDPNSGSTNFADSVKG

SEQ ID NO: 610 - protein, 17 aa, synthetic construct
  REGION 1..17: note = Synthetic: 2193-E09; CDR-H2; Kabat
AIDPGSGYTVPADSVKG

SEQ ID NO: 611 - protein, 17 aa, synthetic construct
  REGION 1..17: note = Synthetic: 2193-B07; CDR-H2; Kabat
AIDPNSGSTLSADSVKG

SEQ ID NO: 612 - protein, 17 aa, synthetic construct
  REGION 1..17: note = Synthetic: 2193-C06; CDR-H2; Kabat
AIDPISGSTQWADSVKG

SEQ ID NO: 613 - protein, 17 aa, synthetic construct
  REGION 1..17: note = Synthetic: 2193-B05; CDR-H2; Kabat
AIDPIGGSTHLADSVKG

SEQ ID NO: 614 - protein, 17 aa, synthetic construct
  REGION 1..17: note = Synthetic: 2193-A11; CDR-H2; Kabat
AIDPKSGSTVYADSVKG

SEQ ID NO: 615 - protein, 17 aa, synthetic construct
  REGION 1..17: note = Synthetic: 2193-E02; CDR-H2; Kabat
SIDPTSGSTVIADSVKG

SEQ ID NO: 616 - protein, 17 aa, synthetic construct
  REGION 1..17: note = Synthetic: 2193-A08; CDR-H2; Kabat
AIDPNAGSTVYADSVKG

SEQ ID NO: 617 - protein, 17 aa, synthetic construct
  REGION 1..17: note = Synthetic: 2193-C11; CDR-H2; Kabat
VIDPKSGSTNYADSVKG

SEQ ID NO: 618 - protein, 17 aa, synthetic construct
  REGION 1..17: note = Synthetic: 2193-A01; CDR-H2; Kabat
AIDPTSGSTVFADSVKG

SEQ ID NO: 619 - protein, 17 aa, synthetic construct
  REGION 1..17: note = Synthetic: 2193-D07; CDR-H2; Kabat
AIGPADGSTVNADSVKG

SEQ ID NO: 620 - protein, 17 aa, synthetic construct
  REGION 1..17: note = Synthetic: 2193-A05; CDR-H2; Kabat
VIDPNSGSTIFADSVKG

SEQ ID NO: 621 - protein, 17 aa, synthetic construct
  REGION 1..17: note = Synthetic: 2193-C03; CDR-H2; Kabat
AIEPKSGSTASADSVKG

SEQ ID NO: 622 - protein, 17 aa, synthetic construct
  REGION 1..17: note = Synthetic: 2193-C09; CDR-H2; Kabat
AIDPISGSTVYADSVKG

SEQ ID NO: 623 - protein, 17 aa, synthetic construct
  REGION 1..17: note = Synthetic: 2193-D01; CDR-H2; Kabat
AIDPTSGPTVYADSVKG

SEQ ID NO: 624 - protein, 17 aa, synthetic construct
  REGION 1..17: note = Synthetic: 2193-E01; CDR-H2; Kabat
AIDPKSGSTAHADSVKG

SEQ ID NO: 625 - protein, 17 aa, synthetic construct
  REGION 1..17: note = Synthetic: 2193-C07; CDR-H2; Kabat
AIDPTRGATVYADSVKG

SEQ ID NO: 626 - protein, 17 aa, synthetic construct
  REGION 1..17: note = Synthetic: 2193-B04; CDR-H2; Kabat
AIEPKNGSTHHADSVKG

SEQ ID NO: 627 - protein, 17 aa, synthetic construct
  REGION 1..17: note = Synthetic: 2193-C10; CDR-H2; Kabat
AIDPRSGSTISADSVKG

SEQ ID NO: 628 - protein, 17 aa, synthetic construct
  REGION 1..17: note = Synthetic: 2193-C01; CDR-H2; Kabat
TIDPKSGSTHVADSVKG

SEQ ID NO: 629 - protein, 17 aa, synthetic construct
  REGION 1..17: note = Synthetic: 2193-A04; CDR-H2; Kabat
AIDPNSGSTLLADSVKG

SEQ ID NO: 630 - protein, 17 aa, synthetic construct
  REGION 1..17: note = Synthetic: 1944-A07; CDR-H2; Kabat
YISPNSGSTYYADSVKG

SEQ ID NO: 631 - protein, 17 aa, synthetic construct
  REGION 1..17: note = Synthetic: 2194-A02; CDR-H2; Kabat
YISPLAGNTYHADSVKG

SEQ ID NO: 632 - protein, 17 aa, synthetic construct
  REGION 1..17: note = Synthetic: 2194-A05; CDR-H2; Kabat
YISPNSGSTYYADSVKG

SEQ ID NO: 633 - protein, 17 aa, synthetic construct
  REGION 1..17: note = Synthetic: 2194-B04; CDR-H2; Kabat
YISPLAGNTYHADSVKG

SEQ ID NO: 634 - protein, 17 aa, synthetic construct
  REGION 1..17: note = Synthetic: 2194-A09; CDR-H2; Kabat
YITPLSGATYRADSVKG

SEQ ID NO: 635 - protein, 17 aa, synthetic construct
  REGION 1..17: note = Synthetic: 2194-A01; CDR-H2; Kabat
YISPLAGNTYHADSVKG

SEQ ID NO: 636 - protein, 17 aa, synthetic construct
  REGION 1..17: note = Synthetic: 2194-B03; CDR-H2; Kabat
YISPLAGNTYHADSVKG

SEQ ID NO: 637 - protein, 17 aa, synthetic construct
  REGION 1..17: note = Synthetic: 2194-B02; CDR-H2; Kabat
YISPLAGNTYHADSVKG

SEQ ID NO: 638 - protein, 17 aa, synthetic construct
  REGION 1..17: note = Synthetic: 2194-A03; CDR-H2; Kabat
YITPNSGTTYSADSVKG

SEQ ID NO: 639 - protein, 17 aa, synthetic construct
  REGION 1..17: note = Synthetic: 2194-A11; CDR-H2; Kabat
YISPNSGYTTDADSVKG

SEQ ID NO: 640 - protein, 17 aa, synthetic construct
  REGION 1..17: note = Synthetic: 2194-A08; CDR-H2; Kabat
YIAPSSGYTYDADSVKG

SEQ ID NO: 641 - protein, 17 aa, synthetic construct
  REGION 1..17: note = Synthetic: 2194-A04; CDR-H2; Kabat
YISPLAGNTHHADSVKG

SEQ ID NO: 642 - protein, 17 aa, synthetic construct
  REGION 1..17: note = Synthetic: 2194-A10; CDR-H2; Kabat
YISPNSGSTHIADSVKG

SEQ ID NO: 643 - protein, 17 aa, synthetic construct
  REGION 1..17: note = Synthetic: 2194-A07; CDR-H2; Kabat
YISPLAGNTYHADSVKG

SEQ ID NO: 644 - protein, 17 aa, synthetic construct
  REGION 1..17: note = Synthetic: 2194-B01; CDR-H2; Kabat
YISPLAGNTYHADSVKG

SEQ ID NO: 645 - protein, 17 aa, synthetic construct
  REGION 1..17: note = Synthetic: 2194-A06; CDR-H2; Kabat
YISPLAGNTYHADSVKG

SEQ ID NO: 646 - protein, 17 aa, synthetic construct
  REGION 1..17: note = Synthetic: 2196-C01; CDR-H2; Kabat
SITPLSGATSKADSVKG

SEQ ID NO: 647 - protein, 17 aa, synthetic construct
  REGION 1..17: note = Synthetic: 2196-A02; CDR-H2; Kabat
QISPNSGATHDADSVKG

SEQ ID NO: 648 - protein, 17 aa, synthetic construct
  REGION 1..17: note = Synthetic: 2196-B03; CDR-H2; Kabat
YISPNSGATHYADSVKG

SEQ ID NO: 649 - protein, 17 aa, synthetic construct
  REGION 1..17: note = Synthetic: 2196-A05; CDR-H2; Kabat
YISPNSGATYQADSVKG

SEQ ID NO: 650 - protein, 17 aa, synthetic construct
  REGION 1..17: note = Synthetic: 2196-C02; CDR-H2; Kabat
FISPNSGWTYSADSVKG

SEQ ID NO: 651 - protein, 17 aa, synthetic construct
  REGION 1..17: note = Synthetic: 2196-B11; CDR-H2; Kabat
QISPNSGYTYYADSVKG

SEQ ID NO: 652 - protein, 17 aa, synthetic construct
  REGION 1..17: note = Synthetic: 2196-B08; CDR-H2; Kabat
QISPNSGYTYLADSVKG

SEQ ID NO: 653 - protein, 17 aa, synthetic construct
  REGION 1..17: note = Synthetic: 2196-A04; CDR-H2; Kabat
QIYPISGHTYQADSVKG

SEQ ID NO: 654 - protein, 17 aa, synthetic construct
  REGION 1..17: note = Synthetic: 2196-A03; CDR-H2; Kabat
YISPNSGSTHEADSVKG

SEQ ID NO: 655 - protein, 17 aa, synthetic construct
  REGION 1..17: note = Synthetic: 2196-B07; CDR-H2; Kabat
YISPNSGATYYADSVKG

SEQ ID NO: 656 - protein, 17 aa, synthetic construct
  REGION 1..17: note = Synthetic: 2196-A06; CDR-H2; Kabat
QISPNSGSTHQADSVKG

SEQ ID NO: 657 - protein, 17 aa, synthetic construct
  REGION 1..17: note = Synthetic: 2196-B05; CDR-H2; Kabat
QISPNSGATYHADSVKG

SEQ ID NO: 658 - protein, 17 aa, synthetic construct
  REGION 1..17: note = Synthetic: 2196-A01; CDR-H2; Kabat
QISPNSGYTYYADSVKG

SEQ ID NO: 659 - protein, 17 aa, synthetic construct
  REGION 1..17: note = Synthetic: 2196-B01; CDR-H2; Kabat
QISPNSGYTYYADSVKG

SEQ ID NO: 660 - protein, 17 aa, synthetic construct
  REGION 1..17: note = Synthetic: 2196-A09; CDR-H2; Kabat
QISPNSGATHYADSVKG

SEQ ID NO: 661 - protein, 17 aa, synthetic construct
  REGION 1..17: note = Synthetic: 2196-A08; CDR-H2; Kabat
YISPNSGSTYKADSVKG

SEQ ID NO: 662 - protein, 17 aa, synthetic construct
  REGION 1..17: note = Synthetic: 2196-A10; CDR-H2; Kabat
YISPNSGATHYADSVKG

SEQ ID NO: 663 - protein, 17 aa, synthetic construct
  REGION 1..17: note = Synthetic: 2196-B06; CDR-H2; Kabat
QISPNSGSTYSADSVKG

SEQ ID NO: 664 - protein, 17 aa, synthetic construct
  REGION 1..17: note = Synthetic: 2196-B09; CDR-H2; Kabat
YISPNSGSTYYADSVKG

SEQ ID NO: 665 - protein, 17 aa, synthetic construct
  REGION 1..17: note = Synthetic: 2196-C03; CDR-H2; Kabat
QISPNSGSTYSADSVKG

SEQ ID NO: 666 - protein, 17 aa, synthetic construct
  REGION 1..17: note = Synthetic: 2196-C04; CDR-H2; Kabat
FIAPLSGSTHNADSVKG

SEQ ID NO: 667 - protein, 17 aa, synthetic construct
  REGION 1..17: note = Synthetic: 2196-A07; CDR-H2; Kabat
QISPNSGSTYYADSVKG

SEQ ID NO: 668 - protein, 17 aa, synthetic construct
  REGION 1..17: note = Synthetic: 2196-A11; CDR-H2; Kabat
QISPNSGTTYDADSVKG

SEQ ID NO: 669 - protein, 17 aa, synthetic construct
  REGION 1..17: note = Synthetic: 2196-B02; CDR-H2; Kabat
YISPNSGQTYDADSVKG

SEQ ID NO: 670 - protein, 17 aa, synthetic construct
  REGION 1..17: note = Synthetic: 2196-B04; CDR-H2; Kabat
YISPNSGATYQADSVKG

SEQ ID NO: 671 - protein, 17 aa, synthetic construct
  REGION 1..17: note = Synthetic: 2196-B10; CDR-H2; Kabat
SITPLSGATSKADSVKG

SEQ ID NO: 672 - protein, 7 aa, synthetic construct
  REGION 1..7: note = Synthetic: 1987-C05; CDR-H3
DYGVFDY

SEQ ID NO: 673 - protein, 7 aa, synthetic construct
  REGION 1..7: note = Synthetic: 2188-D11; CDR-H3
DYGVFDY

SEQ ID NO: 674 - protein, 7 aa, synthetic construct
  REGION 1..7: note = Synthetic: 2188-B04; CDR-H3
DYGVFDY

SEQ ID NO: 675 - protein, 7 aa, synthetic construct
  REGION 1..7: note = Synthetic: 2188-C09; CDR-H3
DYGVFDY

SEQ ID NO: 676 - protein, 7 aa, synthetic construct
  REGION 1..7: note = Synthetic: 2188-G03; CDR-H3
DFGVFDY

SEQ ID NO: 677 - protein, 7 aa, synthetic construct
  REGION 1..7: note = Synthetic: 2188-E03; CDR-H3
DYGVYDY

SEQ ID NO: 678 - protein, 7 aa, synthetic construct
  REGION 1..7: note = Synthetic: 2188-B11; CDR-H3
DYGVFDY

SEQ ID NO: 679 - protein, 7 aa, synthetic construct
  REGION 1..7: note = Synthetic: 2188-E07; CDR-H3
DYGVYDY

SEQ ID NO: 680 - protein, 7 aa, synthetic construct
  REGION 1..7: note = Synthetic: 2188-B02; CDR-H3
DYGVFDY

SEQ ID NO: 681 - protein, 7 aa, synthetic construct
  REGION 1..7: note = Synthetic: 2188-C07; CDR-H3
DYGVFDY

SEQ ID NO: 682 - protein, 7 aa, synthetic construct
  REGION 1..7: note = Synthetic: 2188-A03; CDR-H3
DYGVFDY

SEQ ID NO: 683 - protein, 7 aa, synthetic construct
  REGION 1..7: note = Synthetic: 2188-F03; CDR-H3
DYGVFDY

SEQ ID NO: 684 - protein, 7 aa, synthetic construct
  REGION 1..7: note = Synthetic: 2188-D03; CDR-H3
DYGVYDY

SEQ ID NO: 685 - protein, 7 aa, synthetic construct
  REGION 1..7: note = Synthetic: 2188-C04; CDR-H3
DYGVYDY

SEQ ID NO: 686 - protein, 7 aa, synthetic construct
  REGION 1..7: note = Synthetic: 2188-D10; CDR-H3
DYGVFDY

SEQ ID NO: 687 - protein, 7 aa, synthetic construct
  REGION 1..7: note = Synthetic: 2188-A06; CDR-H3
DYGVFDY

SEQ ID NO: 688 - protein, 7 aa, synthetic construct
  REGION 1..7: note = Synthetic: 2188-C11; CDR-H3
DYGVFDY

SEQ ID NO: 689 - protein, 7 aa, synthetic construct
  REGION 1..7: note = Synthetic: 2188-F01; CDR-H3
DYGVFDY

SEQ ID NO: 690 - protein, 7 aa, synthetic construct
  REGION 1..7: note = Synthetic: 2188-E11; CDR-H3
DYGVFDY

SEQ ID NO: 691 - protein, 7 aa, synthetic construct
  REGION 1..7: note = Synthetic: 2188-A07; CDR-H3
DYGVFDY

SEQ ID NO: 692 - protein, 7 aa, synthetic construct
  REGION 1..7: note = Synthetic: 2188-C01; CDR-H3
DYGVFDY

SEQ ID NO: 693 - protein, 7 aa, synthetic construct
  REGION 1..7: note = Synthetic: 2188-F08; CDR-H3
DYGVFDY

SEQ ID NO: 694 - protein, 7 aa, synthetic construct
  REGION 1..7: note = Synthetic: 2188-E04; CDR-H3
DFGVFDY

SEQ ID NO: 695 - protein, 7 aa, synthetic construct
  REGION 1..7: note = Synthetic: 2188-B01; CDR-H3
DYGVLDY

SEQ ID NO: 696 - protein, 7 aa, synthetic construct
  REGION 1..7: note = Synthetic: 2188-F11; CDR-H3
DYGVFDY

SEQ ID NO: 697 - protein, 7 aa, synthetic construct
  REGION 1..7: note = Synthetic: 2188-B08; CDR-H3
DYGVFDY

SEQ ID NO: 698 - protein, 7 aa, synthetic construct
  REGION 1..7: note = Synthetic: 2188-C10; CDR-H3
DYGVFDY

SEQ ID NO: 699 - protein, 7 aa, synthetic construct
  REGION 1..7: note = Synthetic: 2188-C02; CDR-H3
DYGVFDY

SEQ ID NO: 700 - protein, 7 aa, synthetic construct
  REGION 1..7: note = Synthetic: 2188-B07; CDR-H3
DYGVFDY

SEQ ID NO: 701 - protein, 7 aa, synthetic construct
  REGION 1..7: note = Synthetic: 2188-A11; CDR-H3
DYGVFDY

SEQ ID NO: 702 - protein, 7 aa, synthetic construct
  REGION 1..7: note = Synthetic: 2188-D01; CDR-H3
DYGVFDY

SEQ ID NO: 703 - protein, 7 aa, synthetic construct
  REGION 1..7: note = Synthetic: 2188-E09; CDR-H3
DYGVFDY

SEQ ID NO: 704 - protein, 7 aa, synthetic construct
  REGION 1..7: note = Synthetic: 2188-E06; CDR-H3
DYGVFDY

SEQ ID NO: 705 - protein, 7 aa, synthetic construct
  REGION 1..7: note = Synthetic: 2188-B03; CDR-H3
DYGVYDY

SEQ ID NO: 706 - protein, 7 aa, synthetic construct
  REGION 1..7: note = Synthetic: 2188-F06; CDR-H3
DYGVFDY

SEQ ID NO: 707 - protein, 7 aa, synthetic construct
  REGION 1..7: note = Synthetic: 2188-D02; CDR-H3
DYGVFDY

SEQ ID NO: 708 - protein, 7 aa, synthetic construct
  REGION 1..7: note = Synthetic: 2188-B06; CDR-H3
DYGVFDY

SEQ ID NO: 709 - protein, 7 aa, synthetic construct
  REGION 1..7: note = Synthetic: 2188-D09; CDR-H3
DFGVFDY

SEQ ID NO: 710 - protein, 7 aa, synthetic construct
  REGION 1..7: note = Synthetic: 2188-F02; CDR-H3
DYGVFDY

SEQ ID NO: 711 - protein, 7 aa, synthetic construct
  REGION 1..7: note = Synthetic: 2188-E10; CDR-H3
DYGVFDY

SEQ ID NO: 712 - protein, 7 aa, synthetic construct
  REGION 1..7: note = Synthetic: 2188-A09; CDR-H3
DYGVFDY

SEQ ID NO: 713 - protein, 7 aa, synthetic construct
  REGION 1..7: note = Synthetic: 2188-D04; CDR-H3
DYGVYDY

SEQ ID NO: 714 - protein, 7 aa, synthetic construct
  REGION 1..7: note = Synthetic: 2188-A05; CDR-H3
DYGVFDY

SEQ ID NO: 715 - protein, 7 aa, synthetic construct
  REGION 1..7: note = Synthetic: 2188-E01; CDR-H3
DYGVFDY

SEQ ID NO: 716 - protein, 7 aa, synthetic construct
  REGION 1..7: note = Synthetic: 2188-G01; CDR-H3
DYGVFDY

SEQ ID NO: 717 - protein, 7 aa, synthetic construct
  REGION 1..7: note = Synthetic: 2188-B09; CDR-H3
DYGVFDY

SEQ ID NO: 718 - protein, 7 aa, synthetic construct
  REGION 1..7: note = Synthetic: 2188-F07; CDR-H3
DYGVFDY

SEQ ID NO: 719 - protein, 7 aa, synthetic construct
  REGION 1..7: note = Synthetic: 2188-D08; CDR-H3
DYGVFDY

SEQ ID NO: 720 - protein, 7 aa, synthetic construct
  REGION 1..7: note = Synthetic: 2188-D05; CDR-H3
DYGVYDY

SEQ ID NO: 721 - protein, 7 aa, synthetic construct
  REGION 1..7: note = Synthetic: 2188-C03; CDR-H3
DYGVFDY

SEQ ID NO: 722 - protein, 7 aa, synthetic construct
  REGION 1..7: note = Synthetic: 2188-E08; CDR-H3
DYGVFDY

SEQ ID NO: 723 - protein, 7 aa, synthetic construct
  REGION 1..7: note = Synthetic: 2188-C06; CDR-H3
DYGVFDY

SEQ ID NO: 724 - protein, 7 aa, synthetic construct
  REGION 1..7: note = Synthetic: 2188-A08; CDR-H3
DYGVFDY

SEQ ID NO: 725 - protein, 7 aa, synthetic construct
  REGION 1..7: note = Synthetic: 2188-B10; CDR-H3
DYGVFDY

SEQ ID NO: 726 - protein, 7 aa, synthetic construct
  REGION 1..7: note = Synthetic: 2188-G02; CDR-H3
DYGVFDY

SEQ ID NO: 727 - protein, 7 aa, synthetic construct
  REGION 1..7: note = Synthetic: 2188-C05; CDR-H3
DYGVFDY

SEQ ID NO: 728 - protein, 7 aa, synthetic construct
  REGION 1..7: note = Synthetic: 2188-A10; CDR-H3
DYGVFDY

SEQ ID NO: 729 - protein, 7 aa, synthetic construct
  REGION 1..7: note = Synthetic: 2188-G04; CDR-H3
DYGVYDY

SEQ ID NO: 730 - protein, 7 aa, synthetic construct
  REGION 1..7: note = Synthetic: 2188-C08; CDR-H3
DYGVFDY

SEQ ID NO: 731 - protein, 7 aa, synthetic construct
  REGION 1..7: note = Synthetic: 2188-E05; CDR-H3
DYGVFDY

SEQ ID NO: 732 - protein, 7 aa, synthetic construct
  REGION 1..7: note = Synthetic: 2188-A02; CDR-H3
DFGVFDY

SEQ ID NO: 733 - protein, 7 aa, synthetic construct
  REGION 1..7: note = Synthetic: 2188-F04; CDR-H3
DYGVFDY

SEQ ID NO: 734 - protein, 7 aa, synthetic construct
  REGION 1..7: note = Synthetic: 2188-F05; CDR-H3
DYGVFDY

SEQ ID NO: 735 - protein, 7 aa, synthetic construct
  REGION 1..7: note = Synthetic: 2188-D07; CDR-H3
DYGVFDY

SEQ ID NO: 736 - protein, 7 aa, synthetic construct
  REGION 1..7: note = Synthetic: 2188-E02; CDR-H3
DYGVYDY

SEQ ID NO: 737 - protein, 7 aa, synthetic construct
  REGION 1..7: note = Synthetic: 2188-D06; CDR-H3
DYGVFDY

SEQ ID NO: 738 - protein, 7 aa, synthetic construct
  REGION 1..7: note = Synthetic: 2188-F09; CDR-H3
DYGVFDY

SEQ ID NO: 739 - protein, 7 aa, synthetic construct
  REGION 1..7: note = Synthetic: 2188-F10; CDR-H3
DYGVFDY

SEQ ID NO: 740 - protein, 7 aa, synthetic construct
  REGION 1..7: note = Synthetic: 2188-B05; CDR-H3
DYGVFDY

SEQ ID NO: 741 - protein, 14 aa, synthetic construct
  REGION 1..14: note = Synthetic: 1943-C02; CDR-H3
SRWFRVLWSYVFDY

SEQ ID NO: 742 - protein, 14 aa, synthetic construct
  REGION 1..14: note = Synthetic: 2193-D04; CDR-H3
SRWYRVLWSYAFDY

SEQ ID NO: 743 - protein, 14 aa, synthetic construct
  REGION 1..14: note = Synthetic: 2193-E10; CDR-H3
SRWYRVLWSYVFDY

SEQ ID NO: 744 - protein, 14 aa, synthetic construct
  REGION 1..14: note = Synthetic: 2193-E06; CDR-H3
SRWFRVLWSYVFDY

SEQ ID NO: 745 - protein, 14 aa, synthetic construct
  REGION 1..14: note = Synthetic: 2193-E04; CDR-H3
SRWFRVLWSYVFDY

SEQ ID NO: 746 - protein, 14 aa, synthetic construct
  REGION 1..14: note = Synthetic: 2193-B09; CDR-H3
SRWYRVLWSYVLDY

SEQ ID NO: 747 - protein, 14 aa, synthetic construct
  REGION 1..14: note = Synthetic: 2193-D11; CDR-H3
SRWFRVLWSYVFDY

SEQ ID NO: 748 - protein, 14 aa, synthetic construct
  REGION 1..14: note = Synthetic: 2193-B02; CDR-H3
SRWFRVLWSYVFDY

SEQ ID NO: 749 - protein, 14 aa, synthetic construct
  REGION 1..14: note = Synthetic: 2193-D05; CDR-H3
SRWFRVLWSYVFDY

SEQ ID NO: 750 - protein, 14 aa, synthetic construct
  REGION 1..14: note = Synthetic: 2193-E11; CDR-H3
SRWYRVLWSYVFHY

SEQ ID NO: 751 - protein, 14 aa, synthetic construct
  REGION 1..14: note = Synthetic: 2193-D06; CDR-H3
SRWFRVLWSYVFDY

SEQ ID NO: 752 - protein, 14 aa, synthetic construct
  REGION 1..14: note = Synthetic: 2193-C02; CDR-H3
SRWFRVLWSFVFDY

SEQ ID NO: 753 - protein, 14 aa, synthetic construct
  REGION 1..14: note = Synthetic: 2193-B03; CDR-H3
SRWFRVLWSYVFDY

SEQ ID NO: 754 - protein, 14 aa, synthetic construct
  REGION 1..14: note = Synthetic: 2193-A02; CDR-H3
SRWLRVLWSYIFDY

SEQ ID NO: 755 - protein, 14 aa, synthetic construct
  REGION 1..14: note = Synthetic: 2193-E05; CDR-H3
SRWFRVLWSYVLDY

SEQ ID NO: 756 - protein, 14 aa, synthetic construct
  REGION 1..14: note = Synthetic: 2193-A06; CDR-H3
SRWFRVLWSYIFDY

SEQ ID NO: 757 - protein, 14 aa, synthetic construct
  REGION 1..14: note = Synthetic: 2193-C04; CDR-H3
SRWFRVLWSYVLDY

SEQ ID NO: 758 - protein, 14 aa, synthetic construct
  REGION 1..14: note = Synthetic: 2193-E08; CDR-H3
SRWYRVLWSYVFDY

SEQ ID NO: 759 - protein, 14 aa, synthetic construct
  REGION 1..14: note = Synthetic: 2193-B10; CDR-H3
SRWLRVLWSFVFDY

SEQ ID NO: 760 - protein, 14 aa, synthetic construct
  REGION 1..14: note = Synthetic: 2193-D08; CDR-H3
TRWFRVLWSYVFDY

SEQ ID NO: 761 - protein, 14 aa, synthetic construct
  REGION 1..14: note = Synthetic: 2193-B08; CDR-H3
SRWFRVLWSYALDY

SEQ ID NO: 762 - protein, 14 aa, synthetic construct
  REGION 1..14: note = Synthetic: 2193-C05; CDR-H3
SRWFRVLWSYVIDY

SEQ ID NO: 763 - protein, 14 aa, synthetic construct
  REGION 1..14: note = Synthetic: 2193-D10; CDR-H3
SRWFRVLWSYVFDY

SEQ ID NO: 764 - protein, 14 aa, synthetic construct
  REGION 1..14: note = Synthetic: 2193-D03; CDR-H3
SRWSRVLWIYVFDY

SEQ ID NO: 765 - protein, 14 aa, synthetic construct
  REGION 1..14: note = Synthetic: 2193-A09; CDR-H3
SRWSRVLWSYVFDY

SEQ ID NO: 766 - protein, 14 aa, synthetic construct
  REGION 1..14: note = Synthetic: 2193-A10; CDR-H3
SRWFRVLWSYVFDY

SEQ ID NO: 767 - protein, 14 aa, synthetic construct
  REGION 1..14: note = Synthetic: 2193-E07; CDR-H3
SQWFRVLWSYVFDY

SEQ ID NO: 768 - protein, 14 aa, synthetic construct
  REGION 1..14: note = Synthetic: 2193-A07; CDR-H3
SRWFRVLWSYVFDY

SEQ ID NO: 769 - protein, 14 aa, synthetic construct
  REGION 1..14: note = Synthetic: 2193-E03; CDR-H3
SRWFRVLWSYVFDY

SEQ ID NO: 770 - protein, 14 aa, synthetic construct
  REGION 1..14: note = Synthetic: 2193-C08; CDR-H3
SRWFRVLWSYVFDY

SEQ ID NO: 771 - protein, 14 aa, synthetic construct
  REGION 1..14: note = Synthetic: 2193-B11; CDR-H3
SRWFRVLWSFVFDY

SEQ ID NO: 772 - protein, 14 aa, synthetic construct
  REGION 1..14: note = Synthetic: 2193-A03; CDR-H3
SRWSRVLWTYVFDY

SEQ ID NO: 773 - protein, 14 aa, synthetic construct
  REGION 1..14: note = Synthetic: 2193-D02; CDR-H3
SRWFRVLWSYVFDY

SEQ ID NO: 774 - protein, 14 aa, synthetic construct
  REGION 1..14: note = Synthetic: 2193-B06; CDR-H3
SRWFRVLWTYVFDY

SEQ ID NO: 775 - protein, 14 aa, synthetic construct
  REGION 1..14: note = Synthetic: 2193-B01; CDR-H3
SRWFRVLWSFVLDY

SEQ ID NO: 776 - protein, 14 aa, synthetic construct
  REGION 1..14: note = Synthetic: 2193-D09; CDR-H3
SRWFRVLWSYVFDY

SEQ ID NO: 777 - protein, 14 aa, synthetic construct
  REGION 1..14: note = Synthetic: 2193-E09; CDR-H3
SRWYRVLWSYVFDY

SEQ ID NO: 778 - protein, 14 aa, synthetic construct
  REGION 1..14: note = Synthetic: 2193-B07; CDR-H3
SRWFRVLWSYIFDY

SEQ ID NO: 779 - protein, 14 aa, synthetic construct
  REGION 1..14: note = Synthetic: 2193-C06; CDR-H3
SRWRRALWIYVFDY

SEQ ID NO: 780 - protein, 14 aa, synthetic construct
  REGION 1..14: note = Synthetic: 2193-B05; CDR-H3
SRWYRVLWSYVFDY

SEQ ID NO: 781 - protein, 14 aa, synthetic construct
  REGION 1..14: note = Synthetic: 2193-A11; CDR-H3
SRWFRVLWSYVFDY

SEQ ID NO: 782 - protein, 14 aa, synthetic construct
  REGION 1..14: note = Synthetic: 2193-E02; CDR-H3
SRWWRVLWSYVFDY

SEQ ID NO: 783 - protein, 14 aa, synthetic construct
  REGION 1..14: note = Synthetic: 2193-A08; CDR-H3
SRWFRVLWSYVFDY

SEQ ID NO: 784 - protein, 14 aa, synthetic construct
  REGION 1..14: note = Synthetic: 2193-C11; CDR-H3
SRWFRVLWSYVFDY

SEQ ID NO: 785 - protein, 14 aa, synthetic construct
  REGION 1..14: note = Synthetic: 2193-A01; CDR-H3
SRWFRVLWSYVFDY

SEQ ID NO: 786 - protein, 14 aa, synthetic construct
  REGION 1..14: note = Synthetic: 2193-D07; CDR-H3
SRWLRVLWSYVFDY

SEQ ID NO: 787 - protein, 14 aa, synthetic construct
  REGION 1..14: note = Synthetic: 2193-A05; CDR-H3
SRWFRVLWSYVLDY

SEQ ID NO: 788 - protein, 14 aa, synthetic construct
  REGION 1..14: note = Synthetic: 2193-C03; CDR-H3
SRWYRVLWSYVLDY

SEQ ID NO: 789 - protein, 14 aa, synthetic construct
  REGION 1..14: note = Synthetic: 2193-C09; CDR-H3
SRWSRVLWSYVFDY

SEQ ID NO: 790 - protein, 14 aa, synthetic construct
  REGION 1..14: note = Synthetic: 2193-D01; CDR-H3
SRWFRVLWSYVFDY

SEQ ID NO: 791 - protein, 14 aa, synthetic construct
  REGION 1..14: note = Synthetic: 2193-E01; CDR-H3
SRWFRVLWTYVFDY

SEQ ID NO: 792 - protein, 14 aa, synthetic construct
  REGION 1..14: note = Synthetic: 2193-C07; CDR-H3
SRWYRVLWSFVFDY

SEQ ID NO: 793 - protein, 14 aa, synthetic construct
  REGION 1..14: note = Synthetic: 2193-B04; CDR-H3
SRWFRVLWTYVFDY

SEQ ID NO: 794 - protein, 14 aa, synthetic construct
  REGION 1..14: note = Synthetic: 2193-C10; CDR-H3
SRWFRVLWSFGFDY

SEQ ID NO: 795 - protein, 14 aa, synthetic construct
  REGION 1..14: note = Synthetic: 2193-C01; CDR-H3
SRWFRVLWSYVFDY

SEQ ID NO: 796 - protein, 14 aa, synthetic construct
  REGION 1..14: note = Synthetic: 2193-A04; CDR-H3
SRWFRVLWSYVYDY

SEQ ID NO: 797 - protein, 11 aa, synthetic construct
  REGION 1..11: note = Synthetic: 1944-A07; CDR-H3
DSRRYVRPFDY

SEQ ID NO: 798 - protein, 11 aa, synthetic construct
  REGION 1..11: note = Synthetic: 2194-A02; CDR-H3
DSRRYVRPFDY

SEQ ID NO: 799 - protein, 11 aa, synthetic construct
  REGION 1..11: note = Synthetic: 2194-A05; CDR-H3
DHRRYVRPFDY

SEQ ID NO: 800 - protein, 11 aa, synthetic construct
  REGION 1..11: note = Synthetic: 2194-B04; CDR-H3
DSRRYVRPLDY

SEQ ID NO: 801 - protein, 11 aa, synthetic construct
  REGION 1..11: note = Synthetic: 2194-A09; CDR-H3
DSRRYVRPFDY

SEQ ID NO: 802 - protein, 11 aa, synthetic construct
  REGION 1..11: note = Synthetic: 2194-A01; CDR-H3
DSRRYVRPMDY

SEQ ID NO: 803 - protein, 11 aa, synthetic construct
  REGION 1..11: note = Synthetic: 2194-B03; CDR-H3
DSRRYVRPFDY

SEQ ID NO: 804 - protein, 11 aa, synthetic construct
  REGION 1..11: note = Synthetic: 2194-B02; CDR-H3
DSRRYVRPLDY

SEQ ID NO: 805 - protein, 11 aa, synthetic construct
  REGION 1..11: note = Synthetic: 2194-A03; CDR-H3
DSRRYVRPLDY

SEQ ID NO: 806 - protein, 11 aa, synthetic construct
  REGION 1..11: note = Synthetic: 2194-A11; CDR-H3
DSRRYVRPFDY

SEQ ID NO: 807 - protein, 11 aa, synthetic construct
  REGION 1..11: note = Synthetic: 2194-A08; CDR-H3
DSRRYVRPFDY

SEQ ID NO: 808 - protein, 11 aa, synthetic construct
  REGION 1..11: note = Synthetic: 2194-A04; CDR-H3
DSRRYVRPLDY

SEQ ID NO: 809 - protein, 11 aa, synthetic construct
  REGION 1..11: note = Synthetic: 2194-A10; CDR-H3
DSRRYVRPFDY

SEQ ID NO: 810 - protein, 11 aa, synthetic construct
  REGION 1..11: note = Synthetic: 2194-A07; CDR-H3
DSRRYVRPFDY

SEQ ID NO: 811 - protein, 11 aa, synthetic construct
  REGION 1..11: note = Synthetic: 2194-B01; CDR-H3
DSRRYVRPFDY

SEQ ID NO: 812 - protein, 11 aa, synthetic construct
  REGION 1..11: note = Synthetic: 2194-A06; CDR-H3
DSRRYVRPFDY

SEQ ID NO: 813 - protein, 11 aa, synthetic construct
  REGION 1..11: note = Synthetic: 2196-C01; CDR-H3
DSRRYIRSWDY

SEQ ID NO: 814 - protein, 11 aa, synthetic construct
  REGION 1..11: note = Synthetic: 2196-A02; CDR-H3
DSRRYVRGWDY

SEQ ID NO: 815 - protein, 11 aa, synthetic construct
  REGION 1..11: note = Synthetic: 2196-B03; CDR-H3
DSRRYVRPFDY

SEQ ID NO: 816 - protein, 11 aa, synthetic construct
  REGION 1..11: note = Synthetic: 2196-A05; CDR-H3
DSRRYVRSWDY

SEQ ID NO: 817 - protein, 11 aa, synthetic construct
  REGION 1..11: note = Synthetic: 2196-C02; CDR-H3
DSRRYVRPFDY

SEQ ID NO: 818 - protein, 11 aa, synthetic construct
  REGION 1..11: note = Synthetic: 2196-B11; CDR-H3
DSRRYIRSWDY

SEQ ID NO: 819 - protein, 11 aa, synthetic construct
  REGION 1..11: note = Synthetic: 2196-B08; CDR-H3
DSRRYVRGWDY

SEQ ID NO: 820 - protein, 11 aa, synthetic construct
  REGION 1..11: note = Synthetic: 2196-A04; CDR-H3
ESRRYVGPFGY

SEQ ID NO: 821 - protein, 11 aa, synthetic construct
  REGION 1..11: note = Synthetic: 2196-A03; CDR-H3
DSRRYVRGWDY

SEQ ID NO: 822 - protein, 11 aa, synthetic construct
  REGION 1..11: note = Synthetic: 2196-B07; CDR-H3
DSRRYVRGWDY

SEQ ID NO: 823 - protein, 11 aa, synthetic construct
  REGION 1..11: note = Synthetic: 2196-A06; CDR-H3
DSRRYVRGWDY

SEQ ID NO: 824 - protein, 11 aa, synthetic construct
  REGION 1..11: note = Synthetic: 2196-B05; CDR-H3
DSRRYVRGWDY

SEQ ID NO: 825 - protein, 11 aa, synthetic construct
  REGION 1..11: note = Synthetic: 2196-A01; CDR-H3
DSRRYVRGWDY

SEQ ID NO: 826 - protein, 11 aa, synthetic construct
  REGION 1..11: note = Synthetic: 2196-B01; CDR-H3
DSRRYVRSWDY

SEQ ID NO: 827 - protein, 11 aa, synthetic construct
  REGION 1..11: note = Synthetic: 2196-A09; CDR-H3
DSRRYIRSWDY

SEQ ID NO: 828 - protein, 11 aa, synthetic construct
  REGION 1..11: note = Synthetic: 2196-A08; CDR-H3
DSRRYVRSWDY

SEQ ID NO: 829 - protein, 11 aa, synthetic construct
  REGION 1..11: note = Synthetic: 2196-A10; CDR-H3
DSRRYVRSWDY

SEQ ID NO: 830 - protein, 11 aa, synthetic construct
  REGION 1..11: note = Synthetic: 2196-B06; CDR-H3
DSRRYVLSWDY

SEQ ID NO: 831 - protein, 11 aa, synthetic construct
  REGION 1..11: note = Synthetic: 2196-B09; CDR-H3
DSRRYVRSWDY

SEQ ID NO: 832 - protein, 11 aa, synthetic construct
  REGION 1..11: note = Synthetic: 2196-C03; CDR-H3
DSRRYVLSWDY

SEQ ID NO: 833 - protein, 11 aa, synthetic construct
  REGION 1..11: note = Synthetic: 2196-C04; CDR-H3
ESRRYVNPWDY

SEQ ID NO: 834 - protein, 11 aa, synthetic construct
  REGION 1..11: note = Synthetic: 2196-A07; CDR-H3
DSRRYVRGWDY

SEQ ID NO: 835 - protein, 11 aa, synthetic construct
  REGION 1..11: note = Synthetic: 2196-A11; CDR-H3
DSRRYVRGWDY

SEQ ID NO: 836 - protein, 11 aa, synthetic construct
  REGION 1..11: note = Synthetic: 2196-B02; CDR-H3
DSRRYVRSWDY

SEQ ID NO: 837 - protein, 11 aa, synthetic construct
  REGION 1..11: note = Synthetic: 2196-B04; CDR-H3
DSRRYVRGWDY

SEQ ID NO: 838 - protein, 11 aa, synthetic construct
  REGION 1..11: note = Synthetic: 2196-B10; CDR-H3
DSRRYIRSWDY

SEQ ID NO: 839 - protein, 11 aa, synthetic construct
  REGION 1..11: note = Synthetic: trastuzumab; CDR-L1
RASQDVNTAVA

SEQ ID NO: 840 - protein, 14 aa, synthetic construct
  REGION 1..14: note = Synthetic: SP34; CDR-L1
RSSTGAVTTSNYAN

SEQ ID NO: 841 - protein, 14 aa, synthetic construct
  REGION 1..14: note = Synthetic: 2037-B10; CDR-L1
GSSTGAVTSGYYPN

SEQ ID NO: 842 - protein, 11 aa, synthetic construct
  REGION 1..11: note = Synthetic: hUCHT1-LC3; CDR-L1
RASQDIRNYLN

SEQ ID NO: 843 - protein, 10 aa, synthetic construct
  REGION 1..10: note = Synthetic: hOKT3-LC1; CDR-L1
SASSSVSYMN

SEQ ID NO: 844 - protein, 7 aa, synthetic construct
  REGION 1..7: note = Synthetic: trastuzumab; CDR-L2
SASFLYS

SEQ ID NO: 845 - protein, 7 aa, synthetic construct
  REGION 1..7: note = Synthetic: SP34; CDR-L2
GTNKRAP

SEQ ID NO: 846 - protein, 7 aa, synthetic construct
  REGION 1..7: note = Synthetic: 2037-B10; CDR-L2
GTKFLAP

SEQ ID NO: 847 - protein, 7 aa, synthetic construct
  REGION 1..7: note = Synthetic: hUCHT1-LC3; CDR-L2
YTSRLHS

SEQ ID NO: 848 - protein, 7 aa, synthetic construct
  REGION 1..7: note = Synthetic: hOKT3-LC1; CDR-L2
DTSKLAS

SEQ ID NO: 849 - protein, 9 aa, synthetic construct
  REGION 1..9: note = Synthetic: trastuzumab; CDR-L3
QQHYTTPPT

SEQ ID NO: 850 - protein, 9 aa, synthetic construct
  REGION 1..9: note = Synthetic: SP34; CDR-L3
ALWYSNLWV

SEQ ID NO: 851 - protein, 9 aa, synthetic construct
  REGION 1..9: note = Synthetic: 2037-B10; CDR-L3
ALWYSNRWV

SEQ ID NO: 852 - protein, 9 aa, synthetic construct
  REGION 1..9: note = Synthetic: hUCHT1-LC3; CDR-L3
QQGNTLPWT

SEQ ID NO: 853 - protein, 9 aa, synthetic construct
  REGION 1..9: note = Synthetic: hOKT3-LC1; CDR-L3
QQWSSNPFT

SEQ ID NO: 854 - protein, 116 aa, synthetic construct
  REGION 1..116: note = Synthetic: 1987-C05; VH
EVQLVESGGGLVQPGGSLRLSCAASGFNISDYDIHWVRQAPGKGLEWVGGIDPDDGSTDYADSVKGRFTISADTSKNTAYLQMNSLRAEDTAVYYCARDYGVFDYWGQGTLVTVSS

SEQ ID NO: 855 - protein, 116 aa, synthetic construct
  REGION 1..116: note = Synthetic: 2188-D11; VH
EVQLVESGGGLVQTGGSLRLSCAASGFNIRDYDIHWVRQAPGKGLEWVGLIDPDDGSTDEADSVKGRFTISADTSKNTAYLQMNSLRAEDTAVYYCARDYGVFDYWGQGTLVTVSS

SEQ ID NO: 856 - protein, 116 aa, synthetic construct
  REGION 1..116: note = Synthetic: 2188-B04; VH
EVQLVESGGGLVQPGGSLRLSCAASGFNITWYDIHWVRQAPGKGLEWVGWINPDDGDTYYADSVKGRFTISADTSKNTAYLQMNSLRAEDTAVYYCARDYGVFDYWGQGTLVTVSS

SEQ ID NO: 857 - protein, 116 aa, synthetic construct
  REGION 1..116: note = Synthetic: 2188-C09; VH
EVQLVESGGGLVQPGGSLRLSCAASGFNINSYDIHWVRQAPGKGLEWVGWINPHDGDTYYADSVTGRFTISADTSKNTAYLQMNSLRAEDTAVYYCARDYGVFDYWGQGTLVTVSS

SEQ ID NO: 858 - protein, 116 aa, synthetic construct
  REGION 1..116: note = Synthetic: 2188-G03; VH
EVQLVESGGGLVQPGGSLRLSCAASGFNIVDYDIHWVRQAPGKGLEWVVGIDPRDGSTDYADSVKGRFTISADTSKNTAYLQMNSLRAEDTAVYYCARDFGVFDYWGQGTLVTVSS

SEQ ID NO: 859 - protein, 116 aa, synthetic construct
  REGION 1..116: note = Synthetic: 2188-E03; VH
EVQLVESGGGLVQPGGSLRLSCAASGFNIRDYDIHWVRQAPGKGLEWVAGIDPDDGSTDYADSVKGRFTISADTSKNTAYLQMNSLRAEDTAVYYCARDYGVYDYWGQGTLVTVSS

SEQ ID NO: 860 - protein, 116 aa, synthetic construct
  REGION 1..116: note = Synthetic: 2188-B11; VH
EVQLVESGGGLVQPGGSLRLSCAASGFNINRYDIHWVRQAPGKGLEWVGWINPDDGDTFLADSVKGRFTISADTSKSTAYLQMNSLRAEDTAVYYCARDYGVFDYWGQGTLVTVSS

SEQ ID NO: 861 - protein, 116 aa, synthetic construct
  REGION 1..116: note = Synthetic: 2188-E07; VH
EVQLVESGGGLVQPGGSLRLSCAASGFNIGDYGIHWVRQAPGKGLEWVGGIDPEDGFTVHADSVKGRFTISADTSKNTAYLQMNSLRAEDTAVYYCARDYGVYDYWGQGTLVTVSS

SEQ ID NO: 862 - protein, 116 aa, synthetic construct
  REGION 1..116: note = Synthetic: 2188-B02; VH
EVQLVESGGGLVQPGGSLRLSCAASGFNISSYDIHWVRQAPGKGLEWVGWINPDDGDTYLADSVKGRFTISADTSKSTAYLQMNSLRAEDTAVYYCARDYGVFDYWGQGTLVTVSS

SEQ ID NO: 863 - protein, 116 aa, synthetic construct
  REGION 1..116: note = Synthetic: 2188-C07; VH
EVQLVESGGGLVQPGGSLRLSCAASGFNIVRYDIHWVRQAPGKGLEWVGWINPDDGDTYYADSVKGRFTISADTSKNTAYLQMNSLRAEDTAVYYCARDYGVFDYWGQGTLVTVSS

SEQ ID NO: 864 - protein, 116 aa, synthetic construct
  REGION 1..116: note = Synthetic: 2188-A03; VH
EVQLVESGGGLVQPGGSLRLSCAASGFNIRDYDIHWVRQAPGKGLEWVGWINPDDGDTYYADSVKGRFTISADTSKNTAYLQMNSLRAEDTAVYYCARDYGVFDYWGQGTLVTVSS

SEQ ID NO: 865 - protein, 116 aa, synthetic construct
  REGION 1..116: note = Synthetic: 2188-F03; VH
EVQLVESGGGLVQPGGSLRLSCAASGFNISDYDIHWVRQAPGKGLEWVSGIDPDDGSTDYADSVKGRFTISADTSKNTAYLQMNSLRAEDTAVYYCARDYGVFDYWGQGTLVTVSS

SEQ ID NO: 866 - protein, 116 aa, synthetic construct
  REGION 1..116: note = Synthetic: 2188-D03; VH
EVQLVESGGGLVQPGGSLRLSCAASGFNISDYDIHWVRQAPGKGLEWVGGIDPHDGSTDYADSVKGRFTISADTSKNTAYLQMNSLRAEDTAVYYCARDYGVYDYWGQGTLVTVSS

SEQ ID NO: 867 - protein, 116 aa, synthetic construct
  REGION 1..116: note = Synthetic: 2188-C04; VH
EVQLVESGGGLVQPGGSLRLSCAASGFNINSYDIHWVRQAPGKGLEWVGWINPEDGDTYHADSVRGRFTISADTSKNTAYLQMNSLRAEDTAVYYCARDYGVYDYWGQGTLVTVSS

SEQ ID NO: 868 - protein, 116 aa, synthetic construct
  REGION 1..116: note = Synthetic: 2188-D10; VH
EVQLVESGGGLVQPGGSLRLSCAASGFNISDYDIHWVRQAPGKGLEWVGRIDPRDGATDYADSVKGRFTISADTSKNTAYLQMNSLRAEDTAVYYCARDYGVFDYWGQGTLVTVSS

SEQ ID NO: 869 - protein, 116 aa, synthetic construct
  REGION 1..116: note = Synthetic: 2188-A06; VH
EVQLVESGGGLVQPGGSLRLSCAASGFNIKSYDIHWVRQAPGKGLEWVGWINPDDGDTYHADSVKGRFTISADTSKNTAYLQMNSLRAEDTAVYYCARDYGVFDYWGQGTLVTVSS

SEQ ID NO: 870 - protein, 116 aa, synthetic construct
  REGION 1..116: note = Synthetic: 2188-C11; VH
EVQLVESGGGLVQPGGSLRLSCAASGFNIKSYDIHWVRQAPGKGLEWVGWINPDDGDTYLADSVKGRFTISADTSKNTAYLQMNSLRAEDTAVYYCARDYGVFDYWGQGTLVTVSS

SEQ ID NO: 871 - protein, 116 aa, synthetic construct
  REGION 1..116: note = Synthetic: 2188-F01; VH
EVQLVESGGGLVQPGGSLRLSCAASGFNIRYYDIHWVRQAPGKGLEWVGLIDPDDGWTVSADSVKGRFTISADTSKNTAYLQMNSLRAEDTAVYYCARDYGVFDYWGQGTLVTVSS

SEQ ID NO: 872 - protein, 116 aa, synthetic construct
  REGION 1..116: note = Synthetic: 2188-E11; VH
EVQLVESGGGLVQPGGSLRLSCAASGFNISHYDIHWVRQAPGKGLEWVSGIDPGDGATDHADSVKGRFTISADTSKNTAYLQMNSLRAEDTAVYYCARDYGVFDYWGQGTLVTVSS

SEQ ID NO: 873 - protein, 116 aa, synthetic construct
  REGION 1..116: note = Synthetic: 2188-A07; VH
EVQLVESGGGLVQPGGSLRLSCAASGFNIHHYDIHWVRQAPGKGLEWVGWINPDDGDTYYADSVKGRFTISADTSKNTAYLQMNSLRAEDTAVYYCARDYGVFDYWGQGTLVTVSS

SEQ ID NO: 874 - protein, 116 aa, synthetic construct
  REGION 1..116: note = Synthetic: 2188-C01; VH
EVQLVESGGGLVQPGGSLRLSCAASGFNIISYDIHWVRQAPGKGLEWVSLIDPHDGSTDSADSVKGRFTISADTSKNTAYLQMNSLRAEDTAVYYCARDYGVFDYWGQGTLVTVSS

SEQ ID NO: 875 - protein, 116 aa, synthetic construct
  REGION 1..116: note = Synthetic: 2188-F08; VH
EVQLVESGGGLVQPGGSLRLSCAASGFNIPDFEIHWVRQAPGKGLEWVARIEPDDGATDYADSVKGRFTISADTSKNTAYLQMNSLRAEDTAVYYCARDYGVFDYWGQGTLVTVSS

SEQ ID NO: 876 - protein, 116 aa, synthetic construct
  REGION 1..116: note = Synthetic: 2188-E04; VH
EVQLVESGGGLVQPGGSLRLSCAASGFNISDYDIHWVRQAPGKGLEWVAGIDPQDGSTDYADSVKGRFTISADTSKNTAYLQMNSLRAEDAAVYYCARDFGVFDYWGQGTLVTVSS

SEQ ID NO: 877 - protein, 116 aa, synthetic construct
  REGION 1..116: note = Synthetic: 2188-B01; VH
EVQLVESGGGLVQPGGSLRLSCAASGFNINSHDIHWVRQAPGKGLEWVGWINPDDGDTYYADSVKGRFTISADTSKNTAYLQMNSLRAEDTAVYYCARDYGVLDYWGQGTLVTVSS

SEQ ID NO: 878 - protein, 116 aa, synthetic construct
  REGION 1..116: note = Synthetic: 2188-F11; VH
EVQLVESGGGLVQPGGSLRLSCAASGFNITGYDIHWVRQAPGKGLEWVGGIDPEDGATDYADSVKGRFTISADTSKNTAYLQMNSLRAEDTAVYYCARDYGVFDYWGQGTLVTVSS

SEQ ID NO: 879 - protein, 116 aa, synthetic construct
  REGION 1..116: note = Synthetic: 2188-B08; VH
EVQLVESGGGLVQPGGSLRLSCAASGFNIDSYDIHWVRQAPGKGLEWVGWINPDDGDTFYADSVKGRFTISADTSKNTAYLQMDSLRAEDTAVYYCARDYGVFDYWGQGTLVTVSS

SEQ ID NO: 880 - protein, 116 aa, synthetic construct
  REGION 1..116: note = Synthetic: 2188-C10; VH
EVQLVESGGGLVQPGGSLRLSCAASGFNINSYDIHWVRQAPGKGLEWVGWINPDDGDTYHADSVKGRFTISADTSKNTAYLQMNSLRAEDTAVYYCARDYGVFDYWGQGTLVTVSS

SEQ ID NO: 881 - protein, 116 aa, synthetic construct
  REGION 1..116: note = Synthetic: 2188-C02; VH
EVQLVESGGGLVQPGGSLRLSCAASGFNIERYDIHWVRQAPGKGLEWVGWINPDDGDTYYADSVKGRFTISADTSKNTAYLQTKSLRAEDTAVYYCARDYGVFDYWGQGTLVTVSS

SEQ ID NO: 882 - protein, 116 aa, synthetic construct
  REGION 1..116: note = Synthetic: 2188-B07; VH
EVQLVESGGGLVQPGGSLRLSCAASGFNIHSHDIHWVRQAPGKGLEWVGWINPDDGDTYYADSVKGRFAISADTSKNTAYLQMNSLRAEDTAVYYCARDYGVFDYWGRGTLVTVSS

SEQ ID NO: 883 - protein, 116 aa, synthetic construct
  REGION 1..116: note = Synthetic: 2188-A11; VH
EVQLVESGGGLVQPGGSLRLSCAASGFNISDYDIHWVRQAPGKGLEWVSLIDPKDGATDSADSVKGRFTISADTSKNTAYLQMNSLRAGDTAVYYCARDYGVFDYWGQGTLVTVSS

SEQ ID NO: 884 - protein, 116 aa, synthetic construct
  REGION 1..116: note = Synthetic: 2188-D01; VH
EVQLVESGGGLVQPGGSLRLSCAASGFNIKSYDIHWVRQAPGKGLEWVGWINPYDGDTYYADSVKGRFTISADTSKNTAYLQMNSLRAEDTAVYYCARDYGVFDYWGQGTLVTVSS

SEQ ID NO: 885 - protein, 116 aa, synthetic construct
  REGION 1..116: note = Synthetic: 2188-E09; VH
EVQLVESGGGLVQPGGSLRLSCAASGFNISDFDIHWVRQAPGKGLEWVAEIDPQDGWTVHADSVKGRFTISADTSKNTAYLQMNSLRAEDTAVYYCARDYGVFDYWGQGTLVTVSS

SEQ ID NO: 886 - protein, 116 aa, synthetic construct
  REGION 1..116: note = Synthetic: 2188-E06; VH
EVQLVESGGGLVQPGGSLRLSCAASGFNIGDYDIHWVRQAPGKGLEWVGGIDPEDGATDIADSVKGRFTISADTSKNTAYLQMDSLRAEDTAVYYCARDYGVFDYWGQGTLVTVSS

SEQ ID NO: 887 - protein, 116 aa, synthetic construct
  REGION 1..116: note = Synthetic: 2188-B03; VH
EVQLVESGGGLVQPGGSLRLSCAASGFNISSYDIHWVRQAPGKGLEWVGAIDPGDGATDYADSVKGRFTISADTSKNTAYLQMNSLRAEDTAVYYCARDYGVYDYWGQGTLVTVSS

SEQ ID NO: 888 - protein, 116 aa, synthetic construct
  REGION 1..116: note = Synthetic: 2188-F06; VH
EVQLVESGGGLVQPGGSLRLSCAASGFNILDYDIHWVRQAPGKGLEWVGGIDPDDGATDYADSVKGRFTISADTSKNTAYLQMNSLRAEDTAVYYCARDYGVFDYWGQGTLVTVSS

SEQ ID NO: 889 - protein, 116 aa, synthetic construct
  REGION 1..116: note = Synthetic: 2188-D02; VH
EVQLVESGGGLVQPGGSLRLSCAASGFNISDYDIHWVRQAPGKGLEWVGGIDPEDGATDYADSVKGRFTISADTSKNTAYLQMNSLRAEDTAVYYCARDYGVFDYWGQGTLVTVSS

SEQ ID NO: 890 - protein, 116 aa, synthetic construct
  REGION 1..116: note = Synthetic: 2188-B06; VH
EVQLVESGGGLVQPGGSLRLSCAASGFNIHSYDIHWVRQAPGEGLEWVGWINPDDGDTYHADSVKGRFTISADTSKNTAYLQMNSLRAEDTAVYYCARDYGVFDYWGQGTLVTVSS

SEQ ID NO: 891 - protein, 116 aa, synthetic construct
  REGION 1..116: note = Synthetic: 2188-D09; VH
EVQLVESGGGLVQPGGSLRLSCAASGFNIRDYDIHWVRQAPGKGLEWVVGIDPGDGSTDYADSVKGRFTISADTSKNTAYLQMNSLRAEDTAVYYCARDFGVFDYWGQGTLVTVSS

SEQ ID NO: 892 - protein, 116 aa, synthetic construct
  REGION 1..116: note = Synthetic: 2188-F02; VH
EVQLVESGGGLVQPGGSLRLSCAASGFNISDYDIHWVRQAPGKGLEWVAWIDPSDGSTEHADSVKGRFTISADTSKNTAYLQMSSLRAEDTAVYYCARDYGVFDYWGQGTLVTVSS

SEQ ID NO: 893 - protein, 116 aa, synthetic construct
  REGION 1..116: note = Synthetic: 2188-E10; VH
EVQLVESGGGLVQPGGSLRLSCAASGFNIGDFDIHWVRQAPGKGLEWVGGIDPRDGATDYADSVKGRFTISADTSKNTAYLQMNSLRAEDTAVYYCARDYGVFDYWGQGTLVTVSS

SEQ ID NO: 894 - protein, 116 aa, synthetic construct
  REGION 1..116: note = Synthetic: 2188-A09; VH
EVQLVESGGGLVQPGGSLRLSCAASGFNIINYDIHWVRQAPGKGLEWVGWINPDDGDTYYADSVKGRFTISADTSKNTAYLQMNSLRAEDTAVYYCARDYGVFDYWGQGTLVTVSS

SEQ ID NO: 895 - protein, 116 aa, synthetic construct
  REGION 1..116: note = Synthetic: 2188-D04; VH
EVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSS

SEQ ID NO: 896 - protein, 116 aa, synthetic construct
  REGION 1..116: note = Synthetic: 2188-A05; VH
EVQLVESGGGLVQPGGSLRLSCAASGFNIKRYDIHWVRQAPGKGLEWVGWINPDDGDTYYADSVKGRFTISADTSKNTAYLQMNSLRAEDTAVYYCARDYGVFDYWGQGTLVTVSS

SEQ ID NO: 897 - protein, 116 aa, synthetic construct
  REGION 1..116: note = Synthetic: 2188-E01; VH
EVQLVESGGGLVQPGGSLRLSCAASGFNIGDYDIHWVRQAPGKGLEWVVGIDPQDGATDYADSVKGRFTISADTSKNTAYLQMNSLRAEDTAVYYCARDYGVFDYWGQGTLVTVSS

SEQ ID NO: 898 - protein, 116 aa, synthetic construct
  REGION 1..116: note = Synthetic: 2188-G01; VH
EVQLVESGGGLVQPGGSLRLSCAASGFNISDYDIHWVRQAPGKGLEWVGGIDPNDGATDYADSVKGRFTISADTSKNTAYLQMNSLRAEDTAVYYCARDYGVFDYWGQGTLVTVSS

SEQ ID NO: 899 - protein, 116 aa, synthetic construct
  REGION 1..116: note = Synthetic: 2188-B09; VH
EVQLVESGGGLVQPGGSLRLSCAASGFNINSYDIHWVRQAPGKGLEWVGVIDPKDGWTDHADSVKGRFTISADTSKNTAYLQMNSLRTEDTAVYYCARDYGVFDYWGQGTLVTVSS

SEQ ID NO: 900 - protein, 116 aa, synthetic construct
  REGION 1..116: note = Synthetic: 2188-F07; VH
EVQLVESGGGLVQPGGSLRLSCAASGFNISDYDIHWVRQAPGKGLEWVGGIDPNDGSTDHADSVKGRFTISADTSKNMAYLQMNSLRAEDTAVYYCARDYGVFDYWGQGTLVTVSS

SEQ ID NO: 901 - protein, 116 aa, synthetic construct
  REGION 1..116: note = Synthetic: 2188-D08; VH
EVQLVESGGGLVQPGGSLRLSCAASGFNIRDYDIHWVRQAPGKGLEWVVGIDPDDGATDEADSVKGRFTISADTSKNTAYLQMNSLRAEDTAVYYCARDYGVFDYWGQGTLVTVSS

SEQ ID NO: 902 - protein, 116 aa, synthetic construct
  REGION 1..116: note = Synthetic: 2188-D05; VH
EVQLVESGGGLVQPGGSLRLSCAASGFNISEYDIHWVRQAPGKGLEWVGGIDPHDGWTDHADSVKGRFTISADTSKNTAYLQMNSLRAEDTAVYYCARDYGVYDYWGQGTLVTVSS

SEQ ID NO: 903 - protein, 116 aa, synthetic construct
  REGION 1..116: note = Synthetic: 2188-C03; VH
EVQLVESGGGLVQPGGSLRLSCAASGFNINSYDIHWVRQAPGKGLEWVGWINPDDGDTYYADSVKGRFTISADTSKNTAYLQMNSLRAEDTAVYYCARDYGVFDYWGQGTLVTVSS

SEQ ID NO: 904 - protein, 116 aa, synthetic construct
  REGION 1..116: note = Synthetic: 2188-E08; VH
EVQLVESGGGLVQPGGSLRLSCAASGFNIIDFDIHWVRQAPGKGLEWVGGIDPQDGSTDLADSVKGRLTISADTSKNTAYLQMNSLRAEDTAVYYCARDYGVFDYWGQGTLVTVSS

SEQ ID NO: 905 - protein, 116 aa, synthetic construct
  REGION 1..116: note = Synthetic: 2188-C06; VH
EVQLVESGGGLVQPGGSLRLSCAASGFNINSYDIHWVRQAPGKGLEWVGWINPDDGDTYLADSVKGRFTISADTSKNTAYLQMNSLRAEDTAVYYCARDYGVFDYWGQGTLVTVSS

SEQ ID NO: 906 - protein, 116 aa, synthetic construct
  REGION 1..116: note = Synthetic: 2188-A08; VH
EVQLVESGGGLVQPGGSLRLSCAASGFNIHNFDIHWVRQAPGKGLEWVGWINPFDGDTYYADSVKGRFTISADTSKNTAYLRMNSLRAEDTAAYYCARDYGVFDYWGQGTLVTVSS

SEQ ID NO: 907 - protein, 116 aa, synthetic construct
  REGION 1..116: note = Synthetic: 2188-B10; VH
EVQLVESGGGLVQPGGSLRLSCAASGFNINSYDIHWVRQAPGKGLEWVGWINPHDGDTYHADSVKGRFTISADTSKNTAYLQMNSLRAEDTAVYYCARDYGVFDYWGQGTLVTVSS

SEQ ID NO: 908 - protein, 116 aa, synthetic construct
  REGION 1..116: note = Synthetic: 2188-G02; VH
EVQLVESGGGLVQPGGSLRLSCAASGFNIVGYDIHWVRQAPSKGLEWVGGIDPNDGATDYADSVKGRFTISADTSKNTAYLQMNSLRAEDTAVYYCARDYGVFDYWGQGTLVTVSS

SEQ ID NO: 909 - protein, 116 aa, synthetic construct
  REGION 1..116: note = Synthetic: 2188-C05; VH
EVQLVESGGGLVQPGGSLRLSCAASGFNINSFDIHWVRQAPGKGLEWVGGIDPRDGSTDSADSVKGRFTISADTSKNTAYLQMNSLRAEDTAVYYCARDYGVFDYWGQGTLVTVSS

SEQ ID NO: 910 - protein, 116 aa, synthetic construct
  REGION 1..116: note = Synthetic: 2188-A10; VH
EVQLVESGGGLVQPGGSLRLSCAASGFNINSYDIHWVRQAPGKGLEWVGWINPEDGDTSHADSVKGRFTISADTSKNTAYLQMNSLRAEDTAVYYCARDYGVFDYWGQGTLVTVSS

SEQ ID NO: 911 - protein, 116 aa, synthetic construct
  REGION 1..116: note = Synthetic: 2188-G04; VH
EVQLVESGGGLVQPGGSLRLSCAASGFNIFDYDIHWVRQAPGKGLEWVGGIDPRDGSTDHADSVKGRFTISADTSKNTAYLQMNSLRAEDTAVYYCARDYGVYDYWGQGTLVTVSS

SEQ ID NO: 912 - protein, 116 aa, synthetic construct
  REGION 1..116: note = Synthetic: 2188-C08; VH
EVQLVESGGGLVQPGGSLRLSCAASGFNIKSHDIHWVRQAPGKGLEWVGWINPDDGDTYHADSVKGRFTISADTSKNTAYLQMNSLRAEDTAVYYCARDYGVFDYWGQGTLVTVSS

SEQ ID NO: 913 - protein, 116 aa, synthetic construct
  REGION 1..116: note = Synthetic: 2188-E05; VH
EVQLVESGGGLVQPGGSLRLSCAASGFNISDYDIHWVRQAPGKGLEWVAGIEPWDGSTDHADSVKGRFTISADTSKNTAYLQMNSLRAEDTAVYYCARDYGVFDYWGQGTLVTVSS

SEQ ID NO: 914 - protein, 116 aa, synthetic construct
  REGION 1..116: note = Synthetic: 2188-A02; VH
EVQLVESGGGLVQPGGSLRLSCAASGFNIKSYDIHWVRQAPGKGLEWVGWINPEDGDTYYADSVKGRFTISADTSKNTAYLQMNSLRAEDTAVYYCARDFGVFDYWGQGTLVTVSS

SEQ ID NO: 915 - protein, 116 aa, synthetic construct
  REGION 1..116: note = Synthetic: 2188-F04; VH
EVQLVESGGSLVQPGGSLRLSCAASGFNISDFDIHWVRQAPGKGLEWVSGIDPDDGATDYADSVKGRFTISADTSKNTAYLQMNSLRAEDTAVYYCARDYGVFDYWGQGTLVTVSS

SEQ ID NO: 916 - protein, 116 aa, synthetic construct
  REGION 1..116: note = Synthetic: 2188-F05; VH
EVQLVESGGGLVQPGGSLRLSCAASGFNISDYDIHWVRQAPGKGLEWVGGIDPRDGATDSADSVKGRFTISADTSKNTAYLQMNSLRAEDTAVYYCARDYGVFDYWGQGTLVTVSS

SEQ ID NO: 917 - protein, 116 aa, synthetic construct
  REGION 1..116: note = Synthetic: 2188-D07; VH
EVQLVESGGGLVQPGGSLRLSCAASGFNISDFDIHWVRQAPGKGLEWVVGIDPTDGATDYADSVKGRFTISADTSKNTAYLQMNSLRAEDTAVYYCARDYGVFDYWGQGTLVTVSS

SEQ ID NO: 918 - protein, 116 aa, synthetic construct
  REGION 1..116: note = Synthetic: 2188-E02; VH
EVQLVESGGGLVQPGGSLRLSCAASGFNIPDYEIHWVRQAPGKGLEWVGGIEPHDGSTDYADSVKGRFTISADTSKNTAYLQMNSLRAEDTAVYYCARDYGVYDYWGQGTLVTVSS

SEQ ID NO: 919 - protein, 116 aa, synthetic construct
  REGION 1..116: note = Synthetic: 2188-D06; VH
EVQLVESGGGLVQPGGSLRLSCAASGFNIRDYDIHWVRQAPGKGLEWVGGIDPNDGATDYADSVKGRFTISADTSKNTAYLQMNSLRAEDTAVYYCARDYGVFDYWGQGTLVTVSS

SEQ ID NO: 920 - protein, 116 aa, synthetic construct
  REGION 1..116: note = Synthetic: 2188-F09; VH
EVQLVESGGDLVQPGGSLRLSCAASGFNIRDYDIHWVRQAPGKGLEWVSAIDPNDGSTDYADSVKGRFTISADTSKNTAYLQMNSLRAEDTAVYYCARDYGVFDYWGQGTLVTVSS

SEQ ID NO: 921 - protein, 116 aa, synthetic construct
  REGION 1..116: note = Synthetic: 2188-F10; VH
EVQLVESGGGLVQPGGSLRLSCAASGFNISVFDIHWVRQAPGKGLEWVGGIDPNDGATDHADSVKGRFTISADTSKNTAYLQMNSLRAEDTAVYYCARDYGVFDYWGQGTLVTVSS

SEQ ID NO: 922 - protein, 116 aa, synthetic construct
  REGION 1..116: note = Synthetic: 2188-B05; VH
EVQLVESGGGLVQPGGSLRLSCAASGFNIPDYDIHWVRQAPGKGLEWVGVIDPDDGWTHYADSVKGRFTISADTSKNTAYLQMNSLRAEDTAVYYCARDYGVFDYWGQGTLVTVSS

SEQ ID NO: 923 - protein, 123 aa, synthetic construct
  REGION 1..123: note = Synthetic: 1943-C02; VH
EVQLVESGGGLAQPGGSLRLSCAAPGFNINDYYIHWVRQAPGKGLEWVGAIDPNSGSTDYADSVKGRFAISADTSKNTAYLQMNSLRAEDTAVYYCARSRWFRVLWSYVFDYWGQGTLVTVSS

SEQ ID NO: 924 - protein, 123 aa, synthetic construct
  REGION 1..123: note = Synthetic: 2193-D04; VH
EVQLVESGGGLVQPGGSLRLSCAASGFNINGFYIHWVRQAPGKGLEWVGAIDPNGGSTVYADSVKGRFTISADTSKNTAYLQMNSLRAEDTAVYYCARSRWYRVLWSYAFDYWGQGTLVTVSS

SEQ ID NO: 925 - protein, 123 aa, synthetic construct
  REGION 1..123: note = Synthetic: 2193-E10; VH
EVQLVESGGGLVQPGGSLRLSCAASGFNIYDYYIHWVRQAPGKGLEWVGAIDPVNGSTNNADSVKGRFTISADTSKNTAYLQMNSLRAEDTAVYYCARSRWYRVLWSYVFDYWGQGTLVTVSS

SEQ ID NO: 926 - protein, 123 aa, synthetic construct
  REGION 1..123: note = Synthetic: 2193-E06; VH
EVQLVESGGGLVRPGGSLRLSCAASGFNIDDYYIHWVRQAPGKGLEWVGVIDPKNGSTVYADSVKGRSTISADTPKSTAYLQMNSLRAEDTAVYYCARSRWFRVLWSYVFDYWGQGTLVTVSS

SEQ ID NO: 927 - protein, 123 aa, synthetic construct
  REGION 1..123: note = Synthetic: 2193-E04; VH
EVQLVESGGGLVQPGGSLRLSCAASGFNINDRYIHWVRQAPGKGLEWVGVIDPSLGSTIDADSVKGRFTISADTSRNTAYLQMNSLRAEDTAVYYCARSRWFRVLWSYVFDYWGQGTLVTVSS

SEQ ID NO: 928 - protein, 123 aa, synthetic construct
  REGION 1..123: note = Synthetic: 2193-B09; VH
EVQLVESGGGLVQPGGSLRLSCAASGFNINNSYIHWVRQAPGKGLEWVGIIDPNNGSTAYADSVKGRFTISADTSKNTAYLQMNSLRAEDTAVYYCARSRWYRVLWSYVLDYWGQGTLVTVSS

SEQ ID NO: 929 - protein, 123 aa, synthetic construct
  REGION 1..123: note = Synthetic: 2193-D11; VH
EVQLVESGGGSVQPGGSLRLSCAASGFNINAYYIHWVRQAPGKGLEWVGAIDPNTGSTVDADSVKGRFTISADTSKNTAYLQMNSLRAEDTAVYYCARSRWFRVLWSYVFDYWGQGTLVTVSS

SEQ ID NO: 930 - protein, 123 aa, synthetic construct
  REGION 1..123: note = Synthetic: 2193-B02; VH
EVQLVESGGGLVQPGGSLRLSCAASGFNIKNSYIHWVRQAPGKGLEWVGTIDPTRGSTVHADSVKGRFTISADTSKNTAYLQMNSLRAEDTAVYYCARSRWFRVLWSYVFDYWGQGTLVTVSS

SEQ ID NO: 931 - protein, 123 aa, synthetic construct
  REGION 1..123: note = Synthetic: 2193-D05; VH
EVQLVESGGGLVQPGGSLRLSCAASGFNINDHYIHWVRQAPGKGLEWVGAIDPNSGSTVYADSVKGRFTISADTSKNTAYLQMNSLRAEDTAVYYCARSRWFRVLWSYVFDYWGQGTLVTVSS

SEQ ID NO: 932 - protein, 123 aa, synthetic construct
  REGION 1..123: note = Synthetic: 2193-E11; VH
EVQLVESGGGLVQPGGSLRLSCAASGFNINDYYIHWVRQAPGKGLEWVGGIEPNSGATVFADSVKGRFTIGADTSKNTAYLQMNSLRAEDTAVYYCARSRWYRVLWSYVFHYWGQGTLVTVSS

SEQ ID NO: 933 - protein, 123 aa, synthetic construct
  REGION 1..123: note = Synthetic: 2193-D06; VH
EVQLVESGGGLVQPGGSLRLSCAASGFNIRDYYIHWVRQAPGKGLEWVRAIDPHSGSTVYADSVKGRFTISADTSKNTAYLQMNSLRAEDTAVYYCARSRWFRVLWSYVFDYWGQGTLVTVSS

SEQ ID NO: 934 - protein, 123 aa, synthetic construct
  REGION 1..123: note = Synthetic: 2193-C02; VH
EVQLVESGGGLVQPGGSLRLSCAASGFNISDYYIHWVRQAPGKGLEWVGTIDPQSGSTVYADSVKGRFTISADTSENTAYLQMNSLRAEDTAVYYCARSRWFRVLWSFVFDYWGQGTLVTVSS

SEQ ID NO: 935 - protein, 123 aa, synthetic construct
  REGION 1..123: note = Synthetic: 2193-B03; VH
EVQLVESGGGLVQPGGSLRLSCAASGFNIRDYYIHWVRQAPGKGLEWVGAIDPVSGSTLFADSVKGRFTISADTSKNTAYLQMNSLRAEDTEVYYCARSRWFRVLWSYVFDYWGQGTLVTVSS

SEQ ID NO: 936 - protein, 123 aa, synthetic construct
  REGION 1..123: note = Synthetic: 2193-A02; VH
EVQLVESGGGLVQPGGSLRLSCAASGFNIHASYIHWVRQAPGKGLEWVGAIDPKSGSTYYADSVKGRFTISADTSKNTAYLQMNSLRAEDTAVYYCARSRWLRVLWSYIFDYWGQGTLVTVSS

SEQ ID NO: 937 - protein, 123 aa, synthetic construct
  REGION 1..123: note = Synthetic: 2193-E05; VH
EVQLVESGGGLVQPGGSLRLSCAASGFNIKRSYIHWVRQAPGKGLEWVVSIDPNGSSTHYADSVKGRFTISADTSKNTAYLQMNSLRAEDTAVYYCARSRWFRVLWSYVLDYWGQGTLVTVSS

SEQ ID NO: 938 - protein, 123 aa, synthetic construct
  REGION 1..123: note = Synthetic: 2193-A06; VH
EVQLVESGGGLVQPGGSLRLSCAASGFNINAYYIHWVRQAPGKGLEWVDAIDPDSGSTHNADSVKGRFTISADTSKNTAYLQMNSLRAEDTAVYYCARSRWFRVLWSYIFDYWGQGTLVTVSS

SEQ ID NO: 939 - protein, 123 aa, synthetic construct
  REGION 1..123: note = Synthetic: 2193-C04; VH
EVQLVESGGGLVQPGGSLRLSCAASGFNINTSYIHWVRQAPGKGLEWVGAIDPKSGSTNFADSVKGRFTISADASKNTAYLQMNSLRAEDTAVYYCARSRWFRVLWSYVLDYWGQGTLVTVSS

SEQ ID NO: 940 - protein, 123 aa, synthetic construct
  REGION 1..123: note = Synthetic: 2193-E08; VH
EVQLVESGGGLVQPGGSLRLSCAASGFNINEYYIHWVRQAPGKGLEWVGAIDPHSGSTNFADSVKGRFTISADTSKNTAYLQMNSLRAEDTAVYYCARSRWYRVLWSYVFDYWGQGTLVTVSS

SEQ ID NO: 941 - protein, 123 aa, synthetic construct
  REGION 1..123: note = Synthetic: 2193-B10; VH
EVQLVESGGGLVQPGGSLRLSCAASGFNIADYYVHWVRQAPGKGLEWVGAIDPNSGYTVKADSVKGRFTISADTSKNTAYLQMNSLRAEDTAVYYCARSRWLRVLWSFVFDYWGQGTLVTVSS

SEQ ID NO: 942 - protein, 123 aa, synthetic construct
  REGION 1..123: note = Synthetic: 2193-D08; VH
EVQLVESGGGLVQPGGSLRLSCAASGFNIKRSYIHWVRQAPGKGLEWVVAIDPSGGSTNHADSVKGRFTISADTSKNTAYLQMNSLRAEDTAVYYCARTRWFRVLWSYVFDYWGQGTLVTVSS

SEQ ID NO: 943 - protein, 123 aa, synthetic construct
  REGION 1..123: note = Synthetic: 2193-B08; VH
EVQLVESGGGLVQPGGSLRLSCAASGFNIKDYYIHWVRQAPGKGLEWVSAIDPSPGATLDADSVKGRFTISADTSKNTAYLQMNSLRAEDTAVYYCARSRWFRVLWSYALDYWGQGTLVTVSS

SEQ ID NO: 944 - protein, 123 aa, synthetic construct
  REGION 1..123: note = Synthetic: 2193-C05; VH
EVQLVESGGGLVQPGGSLRLSCAASGFNIKHSYIHWVRQAPGKGLEWVGAIDPHNGSTASADSVKGRFTISADTSKNTAYLQMNSLRAEDTAVHYCARSRWFRVLWSYVIDYWGQGTLVTVSS

SEQ ID NO: 945 - protein, 123 aa, synthetic construct
  REGION 1..123: note = Synthetic: 2193-D10; VH
EVQLVESGGGLVQPGGSLRLSCAASGFNINVSYIHWVRQAPGKGLEWVATIDPNSGFTVHADSVKGRFTISADTSKNTAYLQMNSLRAEDTAVYYCARSRWFRVLWSYVFDYWGQGTLVTVSS

SEQ ID NO: 946 - protein, 123 aa, synthetic construct
  REGION 1..123: note = Synthetic: 2193-D03; VH
EVQLVESGGGLVQPGGSLRLSCAASGFNINDHYIHWVRQAPGKGLEWVSAIDPNTGSTVNADSVKGRFTISADTSKNTAYLQMNSLRAEDTAVYYCARSRWSRVLWIYVFDYWGQGTLVTVSS

SEQ ID NO: 947 - protein, 123 aa, synthetic construct
  REGION 1..123: note = Synthetic: 2193-A09; VH
EVQLVESGGGLVQPGGSLRLSCAASGFNIGKHHIHWVRQAPGKGLEWVGVIDPKGGSTVYADSVKGRFTISADTSKNTAYLQMNSLRAEDTAVYYCARSRWSRVLWSYVFDYWGQGTLVTVSS

SEQ ID NO: 948 - protein, 123 aa, synthetic construct
  REGION 1..123: note = Synthetic: 2193-A10; VH
EVQLVESGGGLVQPGGSLRLSCAASGFNIGDYYIHWVRQAPGKGLEWVGAIDPKSGYTVYADSVKGRFTISADTSKNTAYLQMNSLRAEDTAVYYCARSRWFRVLWSYVFDYWGQGTLVTVSS

SEQ ID NO: 949 - protein, 123 aa, synthetic construct
  REGION 1..123: note = Synthetic: 2193-E07; VH
EVQLVESGGGLVQPGGSLRLSCAASGFNINKVYIHWVRQAPGKGLEWVVSIDPTIGSTHFADSVKGRFTISADTSKSTAYLQMNSLRAEDTAVYYCARSQWFRVLWSYVFDYWGQGTLVTVSS

SEQ ID NO: 950 - protein, 123 aa, synthetic construct
  REGION 1..123: note = Synthetic: 2193-A07; VH
EVQLVESGGGLVQPGGSLRLSCAASGFNINASDIHWVRQAPGKGLGWVGAIDPNTGTTNYADSVKGRFTISADTSKNTAYLQMNSLRAEDTAVYYCARSRWFRVLWSYVFDYWGQGTLVTVSS

SEQ ID NO: 951 - protein, 123 aa, synthetic construct
  REGION 1..123: note = Synthetic: 2193-E03; VH
EVQLVESGGGLVQPGGSLRLSCAASGFNITASYIHWVRQAPGKGLEWVGAIDPKGGSTRFADSVKGRFTISADTSKNTAYLQMNSLRAEDTAVYYCARSRWFRVLWSYVFDYWGQGTLVTVSS

SEQ ID NO: 952 - protein, 123 aa, synthetic construct
  REGION 1..123: note = Synthetic: 2193-C08; VH
EVQLVESGSGLVQPGGSLRLSCAASGFNINHYYIHWVRQAPGKGLEWVGAIDPYPGSTYNADSVKGRFTISADTSKNTAYLQMNSLRAEDTAVYYCARSRWFRVLWSYVFDYWGQGTLVTVSS

SEQ ID NO: 953 - protein, 123 aa, synthetic construct
  REGION 1..123: note = Synthetic: 2193-B11; VH
EVQLVESGGGLVQPGGSLRLSCAASGFNITGSYIHWVRQAPGKGLEWVGAIDPKSGFTSYADSAKGRFTISADTSKNTAYLQMNSLRAEDTAVYYCARSRWFRVLWSFVFDYWGQGTLVTVSS

SEQ ID NO: 954 - protein, 123 aa, synthetic construct
  REGION 1..123: note = Synthetic: 2193-A03; VH
EVQLVESGGGLVQPGGSLRLSCAASGFNIANYHIHWVRQAPGKGLEWVGAIDPKSGSTVHADSVKGRFTISADTSKNTAYLQMNSLRAEDTAVYYCARSRWSRVLWTYVFDYWGQGTLVTVSS

SEQ ID NO: 955 - protein, 123 aa, synthetic construct
  REGION 1..123: note = Synthetic: 2193-D02; VH
EVQLVESGGGLVQPGGSLRLSCAASGFNIKDYYIHWVRQAPGKGLEWVGAIDPESGSTVYADSVKGRFTISADTSKNTAYLQMNSLRAEDTAVYYCARSRWFRVLWSYVFDYWGQGTLVTVSS

SEQ ID NO: 956 - protein, 123 aa, synthetic construct
  REGION 1..123: note = Synthetic: 2193-B06; VH
EVQLVESGGGLVQPGGSLRLSCAASGFNIDGYYIHWVRQAPGKGLEWVAAIDPHPGSTVYADSVKGRFTISADTSKNTAYLQMNSLRAEDTAVYYCARSRWFRVLWTYVFDYWGQGTLVTVSS

SEQ ID NO: 957 - protein, 123 aa, synthetic construct
  REGION 1..123: note = Synthetic: 2193-B01; VH
EVQLVESGGGLVQPGGSLRLSCAASGFNIGGYYIHWVRQAPGKGLEWVGAIDPRSGYTVYADSVKGRFTISADTSKNTAYLQMNSLRAEDTAVYYCARSRWFRVLWSFVLDYWGQGTLVTVSS

SEQ ID NO: 958 - protein, 123 aa, synthetic construct
  REGION 1..123: note = Synthetic: 2193-D09; VH
EVQLVESGGGLVQPGGSLRLSCAASGFNINGYYIHWVRQAPGKGLEWVSAIDPNSGSTNFADSVKGRFTISADTSKNTAYLQMNSLRAEDTAVYYCARSRWFRVLWSYVFDYWGQGTPVTVSS

SEQ ID NO: 959 - protein, 123 aa, synthetic construct
  REGION 1..123: note = Synthetic: 2193-E09; VH
EVQLVESGGGLVQPGGSLRLPCAASGFNIGAYYIHWVRQAPGKGLEWVGAIDPGSGYTVPADSVKGRFTISADTSKNTAYLQMNSLRAEDTAVYYCARSRWYRVLWSYVFDYWGQGTLVTVSS

SEQ ID NO: 960 - protein, 123 aa, synthetic construct
  REGION 1..123: note = Synthetic: 2193-B07; VH
EVQLVESGGGLVQPGGSLRLSCAASGFNINNSYIHWVRQAPGKGLEWVGAIDPNSGSTLSADSVKGRFTISADTSKSTAYLQMNSLRAEDTAVYYCARSRWFRVLWSYIFDYWGQGTLVTVSS

SEQ ID NO: 961 - protein, 123 aa, synthetic construct
  REGION 1..123: note = Synthetic: 2193-C06; VH
EVQLVESGGGLVQPGGSLRLSCAASGFNIIVHYIHWVRQAPGKGLEWVSAIDPISGSTQWADSVKGRFTISADTSKNTAYLQMNSLRAEDTAVYYCARSRWRRALWIYVFDYWGQGTLVTVSS

SEQ ID NO: 962 - protein, 123 aa, synthetic construct
  REGION 1..123: note = Synthetic: 2193-B05; VH
EVQLVESGGGLVQPGGSLRLSCAASGFNINQYYIHWVRQAPGEGLEWVGAIDPIGGSTHLADSVKGRFTISADTSKNTAYLQMNSLRAEDAAVYYCARSRWYRVLWSYVFDYWGQGTLVTVSS

SEQ ID NO: 963 - protein, 123 aa, synthetic construct
  REGION 1..123: note = Synthetic: 2193-A11; VH
EVQLVESGGGLVQPGGSLRLSCAASGFNIDAYYIHWVRQAPGKGLEWVGAIDPKSGSTVYADSVKGRFTTSADTSKNTAYLQMNSLRAEDTAVYYCARSRWFRVLWSYVFDYWGQGTLVTVSS

SEQ ID NO: 964 - protein, 123 aa, synthetic construct
  REGION 1..123: note = Synthetic: 2193-E02; VH
EVQLVESGGGLVQPGGSLRLSCAASGFNIKDYYIHWVRQAPGKGLEWVGSIDPTSGSTVIADSVKGRFTISADTSKNTAYLQMNSLRAEDTAVYYCARSRWWRVLWSYVFDYWGQGTLVTVSS

SEQ ID NO: 965 - protein, 123 aa, synthetic construct
  REGION 1..123: note = Synthetic: 2193-A08; VH
EVQLVESGGGLVQPGGSLRLSCAASGFNIIDSYIHWVRQAPGKGLEWVGAIDPNAGSTVYADSVRGRFTISADTSKNTAYIQMNSLRAEDTAVYYCARSRWFRVLWSYVFDYWGQGTLVTVSS

SEQ ID NO: 966 - protein, 123 aa, synthetic construct
  REGION 1..123: note = Synthetic: 2193-C11; VH
EVQLVESGGGLVQPGGSLRLSCAASGFNIADSYIHWVRQAPGKGLEWVAVIDPKSGSTNYADSVKGRFTISADTSKNTAYLQMNSLRAEDTAVYYCARSRWFRVLWSYVFDYWGQGTLVTVSS

SEQ ID NO: 967 - protein, 123 aa, synthetic construct
  REGION 1..123: note = Synthetic: 2193-A01; VH
EVQLVESGGGLVQPGGSLRLSCAASGFNINDHYIHWVRQAPGKGLEWVGAIDPTSGSTVFADSVKGRFTISADTSKNTAYLQMNSLRAEDTAVYYCARSRWFRVLWSYVFDYWGQGTLVTVSS

SEQ ID NO: 968 - protein, 123 aa, synthetic construct
  REGION 1..123: note = Synthetic: 2193-D07; VH
EVQLVESGGGLVQPGGSLRLSCAASGFNIIDYNIHWVRQAPGKGLEWVTAIGPADGSTVNADSLKGRFTISADTSKNTAYLQMNSLRAEDTAVYYCARSRWLRVLWSYVFDYWGQGTLVTVSS

SEQ ID NO: 969 - protein, 123 aa, synthetic construct
  REGION 1..123: note = Synthetic: 2193-A05; VH
EVQLVESGGGLVQPGGSLRLSCAASGFNIKGYYIHWVRQAPGKGLEWVSVIDPNSGSTIFADSVKGRFTISADTSKNTAYLQMNSLRAEDTAVYYCARSRWFRVLWSYVLDYWGQGTLVTVSS

SEQ ID NO: 970 - protein, 123 aa, synthetic construct
  REGION 1..123: note = Synthetic: 2193-C03; VH
EVQLVESGGGLVQPGGSLRLSCAASGFNINYSYIHWVRQAPGKGLEWVGAIEPKSGSTASADSVKGRFTISADTSKNTAYLQMNSLRAEDAAVYYCARSRWYRVLWSYVLDYWGQGTLVTVSS

SEQ ID NO: 971 - protein, 123 aa, synthetic construct
  REGION 1..123: note = Synthetic: 2193-C09; VH
EVQLVESGGGLVQPGGPLRLSCAASGFNINHSYIHWVRQAPGKGLEWVGAIDPISGSTVYADSVKGRFTISADTSKNTAYLQMNSLRAEDTAVYYCARSRWSRVLWSYVFDYWGQGTLVTVSS

SEQ ID NO: 972 - protein, 123 aa, synthetic construct
  REGION 1..123: note = Synthetic: 2193-D01; VH
EVQLVESGGGLVQPGGSLRLSCAASGFNINDSYIHWVRQAPGKGLEWVGAIDPTSGPTVYADSVKGRFTISADTSKSTAYLQMNSLRAEDTAVYYCARSRWFRVLWSYVFDYWGQGTLVTVSS

SEQ ID NO: 973 - protein, 123 aa, synthetic construct
  REGION 1..123: note = Synthetic: 2193-E01; VH
EVQLVESGGGLVQPGGSLRLSCAASGFNIKGKYIHWVRQAPGKGLEWVGAIDPKSGSTAHADSVKGRFTISADTSKNTAYLQMNSLRAEDTAAYYCARSRWFRVLWTYVFDYWGQGTLVTVSS

SEQ ID NO: 974 - protein, 123 aa, synthetic construct
  REGION 1..123: note = Synthetic: 2193-C07; VH
EVQLVESGGGLVQPGGSLRLSCAASGFNIKDQDIHWVRQAPGKGLEWVGAIDPTRGATVYADSVKGRFTISADTSKNTAYLQMNSLRAEGTAVYYCARSRWYRVLWSFVFDYWGQGTLVTVSS

SEQ ID NO: 975 - protein, 123 aa, synthetic construct
  REGION 1..123: note = Synthetic: 2193-B04; VH
EVQLVESGGGLVQPGGSLRLSCAASGFNISNSYIHWVRQAPGKGLEWVGAIEPKNGSTHHADSVKGRFTISADTSKNTAYLQMNSLRAEDTAVYYCARSRWFRVLWTYVFDYWGQGTLVTVSS

SEQ ID NO: 976 - protein, 123 aa, synthetic construct
  REGION 1..123: note = Synthetic: 2193-C10; VH
EVQLVESGGGLVQPGGSLRLSCAASGFNINNAYIHWVRQAPGKGLEWVGAIDPRSGSTISADSMKGRFTISADTSKNTAYLQMNSLRAEDTAVYYCARSRWFRVLWSFGFDYWGQGTLVTVSS

SEQ ID NO: 977 - protein, 123 aa, synthetic construct
  REGION 1..123: note = Synthetic: 2193-C01; VH
EVQLVESGGGLVQPGGSLRLSCAASGLNINDHYIHWVRQAPGKGLEWVGTIDPKSGSTHVADSVKGRFTISADTSKNTAYLQMNSLRAEDTAVYYCARSRWFRVLWSYVFDYWGQGTLVTVSS

SEQ ID NO: 978 - protein, 123 aa, synthetic construct
  REGION 1..123: note = Synthetic: 2193-A04; VH
EVQLVESGGGLVQPGGSLRLSCAASGFNIISYYIHWVRQAPGKGLEWVGAIDPNSGSTLLADSVKGRFTISADTSKNTAYLQMNSLRAEDTAVYYCARSRWFRVLWSYVYDYWGQGTLVTVSS

SEQ ID NO: 979 - protein, 120 aa, synthetic construct
  REGION 1..120: note = Synthetic: 1944-A07; VH
EVQLVESGGGLVQPGGSLRLSCAASGFNITGYYIHWVRQAPGKGLEWVGYISPNSGSTYYADSVKGRFTISADTSKNTAYLQMNSLRAEDTAVYYCARDSRRYVRPFDYWGQGTLVTVSS

SEQ ID NO: 980 - protein, 120 aa, synthetic construct
  REGION 1..120: note = Synthetic: 2194-A02; VH
EVQLVESGGGLVQPGGSLRLSCAASGFNITGYYIHWVRQAPGKGLEWVGYISPLAGNTYHADSVKGRFTISADTSKNTAYLQMNSLRAEDTAVYYCARDSRRYVRPFDYWGQGTLVTVSS

SEQ ID NO: 981 - protein, 120 aa, synthetic construct
  REGION 1..120: note = Synthetic: 2194-A05; VH
EVQLVESGGGLVQPGGSLRLSCAASGFNIKEYHIHWVRQAPGKGLEWVGYISPNSGSTYYADSVKGRFTISADTSKNTAYLQMNSLRAEDTAVYYCARDHRRYVRPFDYWGQGTLVTVSS

SEQ ID NO: 982 - protein, 120 aa, synthetic construct
  REGION 1..120: note = Synthetic: 2194-B04; VH
EVQLVESGGGLVQPGGSLRLSCAASGFNITGYHIHWVRQAPGKGLEWVGYISPLAGNTYHADSVKGRFTISADTSKNTAYLQMNSLRAEDTAVYYCTRDSRRYVRPLDYWGQGTLVTVSS

SEQ ID NO: 983 - protein, 120 aa, synthetic construct
  REGION 1..120: note = Synthetic: 2194-A09; VH
EVQLVESGGGLVQPGGSLRLSCAASGFNITGYYIHWVRQAPGKGLEWVGYITPLSGATYRADSVKGRFTISADTSKNTAYLQMNSLRAEDTAVYYCARDSRRYVRPFDYWGQGTLVTVSS

SEQ ID NO: 984 - protein, 120 aa, synthetic construct
  REGION 1..120: note = Synthetic: 2194-A01; VH
EVQLVESGGGLVQPGGSLRLSCAASGFNITGYYIHWVRQAPGKGLEWVGYISPLAGNTYHADSVKGRFNISADTSKNTAYLQMNSLRAEDTAVYYCARDSRRYVRPMDYWGQGTLVTVSS

SEQ ID NO: 985 - protein, 120 aa, synthetic construct
  REGION 1..120: note = Synthetic: 2194-B03; VH
EVQLVESGGGLVQPGGSLRLSCAASGFNITSYYIHWVRQAPGKGLEWVGYISPLAGNTYHADSVKGRFTISADTSKNTAYLQMNSLRAEDTAVYYCARDSRRYVRPFDYWGQGTLVTVSS

SEQ ID NO: 986 - protein, 120 aa, synthetic construct
  REGION 1..120: note = Synthetic: 2194-B02; VH
EVQLVESGGGLVQPGGSLRLSCAASGFNISGYYIHWVRQTPGKGLEWVGYISPLAGNTYHADSVKGRFTISADTSKNTAYLQMNSLRAEDTAVYYCARDSRRYVRPLDYWGQGTLVTVSS

SEQ ID NO: 987 - protein, 120 aa, synthetic construct
  REGION 1..120: note = Synthetic: 2194-A03; VH
EVQLVESGGGLVQPGGPLRLSCAASGFNITGYYIHWVRQAPGKGLEWVGYITPNSGTTYSADSVKGRFTISADTSKNTAYLQMNSLRAEDTAVYYCARDSRRYVRPLDYWGQGTLVTVSS

SEQ ID NO: 988 - protein, 120 aa, synthetic construct
  REGION 1..120: note = Synthetic: 2194-A11; VH
EVQLVESGGGLVQPGGSLRLSCAASGFNITQYYIHWVRQAPGKGLEWVGYISPNSGYTTDADSVKGRFTISADTSKNTAYLQMNSLRAEDTAVYYCARDSRRYVRPFDYWGQGTLVTVSS

SEQ ID NO: 989 - protein, 120 aa, synthetic construct
  REGION 1..120: note = Synthetic: 2194-A08; VH
EVQLVESGGGLVQPGGSLRLSCAASGFNITGYHIHWVRQAPGKGLEWVGYIAPSSGYTYDADSVKGRFTISADTSKNTAYLQMNSLRAEDTAVYYCARDSRRYVRPFDYWGQGTLVTVSS

SEQ ID NO: 990 - protein, 120 aa, synthetic construct
  REGION 1..120: note = Synthetic: 2194-A04; VH
EVQLVESGGGLVQPGGSLRLSCAASGFNITGYYIHWVRQAPGKGLEWVGYISPLAGNTHHADSVKGRFTISADTSKNTAYLQMNSLRAEDTAVYYCARDSRRYVRPLDYWGQGTLVTVSS

SEQ ID NO: 991 - protein, 120 aa, synthetic construct
  REGION 1..120: note = Synthetic: 2194-A10; VH
EVQLVESGGGLVQPGGSLRLSCAASGFNITQYYIHWVRQAPAKGLEWVGYISPNSGSTHIADSVKGRFTISADTSKNTAYLQMNSLRAEDTAVYYCARDSRRYVRPFDYWGQSTLVTVSS

SEQ ID NO: 992 - protein, 120 aa, synthetic construct
  REGION 1..120: note = Synthetic: 2194-A07; VH
EVQLVESGGGLVQPGGSLRLSCAASGFNFTDYYIHWVRQAPGKGLEWVGYISPLAGNTYHADSVKGRFTISADTSKNTAYLQMNSLRAEDTAVYYCARDSRRYVRPFDYWGQGTLVTVSS

SEQ ID NO: 993 - protein, 120 aa, synthetic construct
  REGION 1..120: note = Synthetic: 2194-B01; VH
EVQLVESGGGLVQPGGSLRLSCAASGFNIASYYIHWVRQAPGKGLEWVGYISPLAGNTYHADSVKGRFTISADTSKNTAYLQMNSLRAEDTAVYYCARDSRRYVRPFDYWGQGTLVTVSS

SEQ ID NO: 994 - protein, 120 aa, synthetic construct
  REGION 1..120: note = Synthetic: 2194-A06; VH
EVQLVESGGGLVQPGGSLRLSCAASGFNITDYYIHWVRQAPGKGLEWVGYISPLAGNTYHADSVKGRFPISADTSKNTAYLQMNSLRAEDTAVYYCARDSRRYVRPFDYWGQGTLVTVSS

SEQ ID NO: 995 - protein, 120 aa, synthetic construct
  REGION 1..120: note = Synthetic: 2196-C01; VH
EVQLVESGGGLVQPGGSLRLSCAASGFNIIGYYIHWVRQAPGKGLEWVGSITPLSGATSKADSVKGRFTISADTSKNTAYLQMNSLRAEDTAVYYCARDSRRYIRSWDYWGQGTLVTVSS

SEQ ID NO: 996 - protein, 120 aa, synthetic construct
  REGION 1..120: note = Synthetic: 2196-A02; VH
EVQLVESGGGLVQPGGSLRLSCAASGFNITGYYIHWVRQAPGKGLEWVGQISPNSGATHDADSVKGRFTISADTSKNTAYLQMNSLRAEDTAVYYCARDSRRYVRGWDYWGQGTLVTVSS

SEQ ID NO: 997 - protein, 120 aa, synthetic construct
  REGION 1..120: note = Synthetic: 2196-B03; VH
EVQLVESGGGLAQPGGSLRLSCAASGFNITGYYIHWVRQAPGKGLEWVGYISPNSGATHYADSVKGRFTISADTSKNTAYLQMNSLRAEDTAVYYCARDSRRYVRPFDYWGQGTLVTVSS

SEQ ID NO: 998 - protein, 120 aa, synthetic construct
  REGION 1..120: note = Synthetic: 2196-A05; VH
EVQLVESGGGLVQPGGSLRLSCAASGFNITNYYIHWVRQAPGKGLEWVGYISPNSGATYQADSVKGRFTISADTSKNTAYLQMNSLRAEDTAVYYCARDSRRYVRSWDYWGQGTLVTVSS

SEQ ID NO: 999 - protein, 120 aa, synthetic construct
  REGION 1..120: note = Synthetic: 2196-C02; VH
EVQLVESGGGLVQPGGSLRLSCAASGFNITGYHIHWVRQAPGKGLEWVGFISPNSGWTYSADSVKGRFTISADTSKNTAYLQMNSLRAEDTAVYYCARDSRRYVRPFDYWGQGTLVTVSS

SEQ ID NO: 1000 - protein, 120 aa, synthetic construct
  REGION 1..120: note = Synthetic: 2196-B11; VH
EVQLVESGGGLVQPGGSLRLSCAASGFNITGYYIHWVRQAPGRGLEWVGQISPNSGYTYYADSVKGRFTISADTSKNTAYLQMNSLRAEDTAVYYCARDSRRYIRSWDYWGQGTLVTVSS

SEQ ID NO: 1001 - protein, 120 aa, synthetic construct
  REGION 1..120: note = Synthetic: 2196-B08; VH
EVQLVESGGGLVQPGGSLRLSCAASGFNITGYYIHWVRQAPGKGLEWVGQISPNSGYTYLADSVKGRFTISADTSKNTAYLQMNSLRAEDTAVYYCARDSRRYVRGWDYWGQGTLVTVSS

SEQ ID NO: 1002 - protein, 120 aa, synthetic construct
  REGION 1..120: note = Synthetic: 2196-A04; VH
EVQLVESGGGLVQPGGSLRLSCAASGFNISPYYIHWVRQAPGKGLEWVGQIYPISGHTYQADSVKGRFTISADTSKNTAYLQMNSLRAEDTAVYYCARESRRYVGPFGYWGQGTLVTVSS

SEQ ID NO: 1003 - protein, 120 aa, synthetic construct
  REGION 1..120: note = Synthetic: 2196-A03; VH
EVQLVESGGGLVQPGGSLRLSCAASGFNITQYHIHWVRQAPGKGLEWVGYISPNSGSTHEADSVKGRFTISADTSKNTAYLQMNSLRAEDTAVYYCARDSRRYVRGWDYWGQGTLVTVSS

SEQ ID NO: 1004 - protein, 120 aa, synthetic construct
  REGION 1..120: note = Synthetic: 2196-B07; VH
EVQLVESGGGLVQPGGSLRLSCAASGFNITGYHIHWVRQAPGEGLEWVGYISPNSGATYYADSVEGRFTISADTSKNTAYLQMHSLRAEDTAVYYCTRDSRRYVRGWDYWGQGTLVTVSS

SEQ ID NO: 1005 - protein, 120 aa, synthetic construct
  REGION 1..120: note = Synthetic: 2196-A06; VH
EVQLVESGGGLVQPGGSLRLSCAASGFNITGYYIHWVRQAPGKGLEWVGQISPNSGSTHQADSVKGRFTISADTSKNTAYLQMNSLRAGDTAVYYCARDSRRYVRGWDYWGQGTLVTVSS

SEQ ID NO: 1006 - protein, 120 aa, synthetic construct
  REGION 1..120: note = Synthetic: 2196-B05; VH
EVQLVESGGGLVQPGGSLRLSCAASGFNITGYYIHWVRQAPGKGLEWVGQISPNSGATYHADSVKGRFTISADTSKNTAYLQMNSLRAEDTAVYYCARDSRRYVRGWDYWGQGTLVTVSS

SEQ ID NO: 1007 - protein, 120 aa, synthetic construct
  REGION 1..120: note = Synthetic: 2196-A01; VH
EVQLVESGGGLVQPGGSLRLSCAASGFNITGYYIHWVRQAPGKGLEWVGQISPNSGYTYYADSVKGRFTISADTSKNTAYLQMNSLRAEDTAVYYCARDSRRYVRGWDYWGQGTLVTVSS

SEQ ID NO: 1008 - protein, 120 aa, synthetic construct
  REGION 1..120: note = Synthetic: 2196-B01; VH
EVQLVESGGGLVQPGGSLRLSCAASGFNITGYYIHWVRQAPGKGLEWVGQISPNSGYTYYADSVKGRFTISADTSKNTTYLQMNSLRAEDTAVYYCARDSRRYVRSWDYWGQGTLVTVSS

SEQ ID NO: 1009 - protein, 120 aa, synthetic construct
  REGION 1..120: note = Synthetic: 2196-A09; VH
EVQLVESGGGLVQPGGSLRLSCAASGFNITGYYIHWVRQAPGKGLEWVGQISPNSGATHYADSVKGRFTISADTSKNTAYLQMNSLRAEDTAVYYCARDSRRYIRSWDYWGQGTLVTVSS

SEQ ID NO: 1010 - protein, 120 aa, synthetic construct
  REGION 1..120: note = Synthetic: 2196-A08; VH
EVQLVESGGGLVQPGGSLRLSCAASGFNITGYYIHWVRQAPGKGLEWVGYISPNSGSTYKADSVKGRFTISADTSRNTAYLQMNSLRAEDTAVYYCARDSRRYVRSWDYWGQGTLVTVSS

SEQ ID NO: 1011 - protein, 120 aa, synthetic construct
  REGION 1..120: note = Synthetic: 2196-A10; VH
EVQLVESGGGLVQPGGSLRLSCAASGFNITGYYIHWVRQAPGKGLEWVGYISPNSGATHYADSVKGRFTISADISKNTAYLQMNSLRAEDTAVHYCARDSRRYVRSWDYWGQGTLVTVSS

SEQ ID NO: 1012 - protein, 120 aa, synthetic construct
  REGION 1..120: note = Synthetic: 2196-B06; VH
EVQLVESGGGLVQPGGSLRLSCAASGFNITGYYIHWVRQAPGKGLEWVGQISPNSGSTYSADSVKGRFTISADTSKNTAYLQMNSLRAEDTAVYYCARDSRRYVLSWDYWGQGTLVTVSS

SEQ ID NO: 1013 - protein, 120 aa, synthetic construct
  REGION 1..120: note = Synthetic: 2196-B09; VH
EVQLVESGGGLVQPEGSLRLSCAASGFNITEYYIHWVRQAPGKGLEWVGYISPNSGSTYYADSVKGRFTISADTSKNTAYLQMNSLRAEDTAVYYCARDSRRYVRSWDYWGQGTLVTVSS

SEQ ID NO: 1014 - protein, 120 aa, synthetic construct
  REGION 1..120: note = Synthetic: 2196-C03; VH
EVQLVESGGGLVQPGGSLRLSCAASGFNITGYYIHWVRQAPGKGLEWVGQISPNSGSTYSADSVKGRFTISADTSKNTAYLQMSSLRAEDTAVYYCARDSRRYVLSWDYWGQGTLVTVSS

SEQ ID NO: 1015 - protein, 120 aa, synthetic construct
  REGION 1..120: note = Synthetic: 2196-C04; VH
EVQLVESGGGLVQPGGSLRLSCAASGFNITGYYIHWVRQAPGKGLEWVGFIAPLSGSTHNADSVKGRFTISADTSKNTAYLQMNSLRAEDTAVYYCARESRRYVNPWDYWGQGTLVTVSS

SEQ ID NO: 1016 - protein, 120 aa, synthetic construct
  REGION 1..120: note = Synthetic: 2196-A07; VH
EVQLVESGGGLVQPGGSLRLSCAASGFNITGYYIHWVRQAPGKGLEWVGQISPNSGSTYYADSVKGRFTISADTSKNTAYLQMSSLRAEDTAVYYCARDSRRYVRGWDYWGQGTLVTVSS

SEQ ID NO: 1017 - protein, 120 aa, synthetic construct
  REGION 1..120: note = Synthetic: 2196-A11; VH
EVQLVESGGGLVQPGGSLRLSCAASGFNITGYYIHWVRQAPGKGLEWVGQISPNSGTTYDADSVKGRFTISADTSKNTAYLQMNSLRAEDTAVYYCARDSRRYVRGWDYWGQGTLVTVSS

SEQ ID NO: 1018 - protein, 120 aa, synthetic construct
  REGION 1..120: note = Synthetic: 2196-B02; VH
EVQLVESGGGLVQPGGSLRLSCAASGFNITQYYIHWVRQAPGKGLEWVGYISPNSGQTYDADSVKGRFTISADTSKNTAYLQMNSLRAEDTAVYYCARDSRRYVRSWDYWGQGTLVTVSS

SEQ ID NO: 1019 - protein, 120 aa, synthetic construct
  REGION 1..120: note = Synthetic: 2196-B04; VH
EVQLVESGGGLVQPGGSLRLSCAASGFNITEYYIHWVRQAPGKGLEWVGYISPNSGATYQADSVKGRFTISADTSKNTAYLQMNSLRAEDTAVYYCARDSRRYVRGWDYWGQGTLVTVSS

SEQ ID NO: 1020 - protein, 120 aa, synthetic construct
  REGION 1..120: note = Synthetic: 2196-B10; VH
EVQLVESGGGLVQPGGSLRLSCAASGFNITGYYIHWVRQAPGKGLEWVGSITPLSGATSKADSVKGRFTISADTSKNTAYLQMNSLRAEDTAVYYCARDSRRYIRSWDYWGQGTLVTVSS

SEQ ID NO: 1021 - protein, 107 aa, synthetic construct [sequence omitted]

SEQ ID NO: 1022 - protein, 109 aa, synthetic construct
  REGION 1..109: note = Synthetic: SP34 ; VL
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 1023 - protein, 110 aa, synthetic construct
  REGION 1..110: note = Synthetic: 2037-B10; VL
QTVVTQEPSLTVSPGGTVTLTCGSSTGAVTSGYYPNWLQQKPGQAPRGLIGGTKFLAPGTPARFSGSLLGGKAALTLSGVQPEDEAEYYCALWYSNRWVFGGGTQLTVTG

SEQ ID NO: 1024 - protein, 107 aa, synthetic construct
  REGION 1..107: note = Synthetic: hUCHT1-LC3; VL
DIQMTQSPSTLSASVGDRVTITCRASQDIRNYLNWYQQKPGKAPKLLIYYTSRLHSGVPSRFSGSGSGTEYTLTISSLQPDDFATYYCQQGNTLPWTFGGGTKVEIK

SEQ ID NO: 1025 - protein, 106 aa, synthetic construct
  REGION 1..106: note = Synthetic: hOKT3-LC1; VL
DIQMTQSPSSLSASVGDRVTITCSASSSVSYMNWYQQKPGKAPKRLIYDTSKLASGVPSRFSGSGSGTEYTLTISSLQPEDFATYYCQQWSSNPFTFGQGTKLEIK

SEQ ID NO: 1026 - protein, 110 aa, synthetic construct
  REGION 1..110: note = Synthetic: hSP34-LC3; VL
QTVVTQEPSLTVSPGGTVTLTCGSSTGAVTSGYYPNWFQQKPGQAPRGLIGGTKFLAPGTPARFSGSLLGGKAALTLSGVQPEDEAEYYCALWYSNRWVFGGGTQLTVTG

SEQ ID NO: 1027 - protein, 330 aa, synthetic construct
  REGION 1..330: note = Synthetic: Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1028 - protein, 107 aa, synthetic construct [sequence omitted]

SEQ ID NO: 1029 - protein, 323 aa, synthetic construct
  REGION 1..323: note = Synthetic: Mouse IgG1 HC Constant
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 1030 - protein, 107 aa, synthetic construct
  REGION 1..107: note = Synthetic: Mouse IgG LC Constant Ckappa
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 1031 - protein, 108 aa, synthetic construct
  REGION 1..108: note = Synthetic: Kappa LC
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1032 - protein, 106 aa, synthetic construct
  REGION 1..106: note = Synthetic: Lambda LD
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1033 - protein, 20 aa, synthetic construct
  REGION 1..20: note = Synthetic: FlagHis Tag
GSGDYKDDDDKGSGHHHHHH

SEQ ID NO: 1034 - protein, 15 aa, synthetic construct
  REGION 1..15: note = Synthetic: Linker
GGGGSGGGGSGGGGS

SEQ ID NO: 1035 - protein, 11 aa, synthetic construct
  REGION 1..11: note = Synthetic: Linker
AAGSDQEPKSS

SEQ ID NO: 1036 - protein, 445 aa, synthetic construct
  REGION 1..445: note = Synthetic: 2188-D04 IgG HC Y180F404TAG
MEVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 1037 - protein, 446 aa, synthetic construct
  REGION 1..446: note = Synthetic: 2188-D04 Y180TAG V262E
MEVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1038 - protein, 446 aa, synthetic construct
  REGION 1..446: note = Synthetic: 2188-D04 IgG HC F404TAG
MEVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 1039 - protein, 446 aa, synthetic construct
  REGION 1..446: note = Synthetic: 2188-D04_HC_F404TAG_Knob
MEVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFLYSKLTVDKSRWQQGNVFSCSVMHEALHNHYTQKSLSLSPGK

SEQ ID NO: 1040 - protein, 446 aa, synthetic construct
  REGION 1..446: note = Synthetic: 2188-D04_HC_F404TAG_Hole
MEVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFLVSKLTVDKSRWQQGNVFSCSVMHEALHNHYTQKSLSLSPGK

SEQ ID NO: 1041 - protein, 450 aa, synthetic construct
  REGION 1..450: note = Synthetic: 2194-A05_HC_F404TAG_Knob
MEVQLVESGGGLVQPGGSLRLSCAASGFNIKEYHIHWVRQAPGKGLEWVGYISPNSGSTYYADSVKGRFTISADTSKNTAYLQMNSLRAEDTAVYYCARDHRRYVRPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFLYSKLTVDKSRWQQGNVFSCSVMHEALHNHYTQKSLSLSPGK

SEQ ID NO: 1042 - protein, 450 aa, synthetic construct; identical to SEQ ID NO: 1041
  REGION 1..450: note = Synthetic: 1987-C06_HC_F404TAG_Hole

SEQ ID NO: 1043 - protein, 446 aa, synthetic construct
  REGION 1..446: note = Synthetic: 1943-C01_HC_F404TAG_Hole
MEVQLVESGGGLVQPGGSLRLSCAASGFNINGYDIHWVRQAPGKGLEWVGYIDPNDGATNYADSVKGRFTISADTSKNTAYLQMNSLRAEDTAVYYCARDYGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFLVSKLTVDKSRWQQGNVFSCSVMHEALHNHYTQKSLSLSPGK

SEQ ID NO: 1044 - protein, 453 aa, synthetic construct
  REGION 1..453: note = Synthetic: 2193-B09_HC_F404TAG_Hole
MEVQLVESGGGLVQPGGSLRLSCAASGFNINNSYIHWVRQAPGKGLEWVGIIDPNNGSTAYADSVKGRFTISADTSKNTAYLQMNSLRAEDTAVYYCARSRWYRVLWSYVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFLVSKLTVDKSRWQQGNVFSCSVMHEALHNHYTQKSLSLSPGK

SEQ ID NO: 1045 - protein, 450 aa, synthetic construct
  REGION 1..450: note = Synthetic: 2194-A05_HC_F404TAG_Hole
MEVQLVESGGGLVQPGGSLRLSCAASGFNIKEYHIHWVRQAPGKGLEWVGYISPNSGSTYYADSVKGRFTISADTSKNTAYLQMNSLRAEDTAVYYCARDHRRYVRPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFLVSKLTVDKSRWQQGNVFSCSVMHEALHNHYTQKSLSLSPGK

SEQ ID NO: 1046 - protein, 453 aa, synthetic construct
  REGION 1..453: note = Synthetic: 2186-B04_HC_F404TAG_Hole
MEVQLVESGGGLVQPGGSLRLSCAASGFNIAGYAIHWVRQAPGKGLEWVGLITPTPGTSNYADSVKGRFTISVDTSKNTAYLQMNSLRAEDTAAYYCARDYRGVYLYSFYYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFLVSKLTVDKSRWQQGNVFSCSVMHEALHNHYTQKSLSLSPGK

SEQ ID NO: 1047 - protein, 453 aa, synthetic construct
  REGION 1..453: note = Synthetic: 1987-C06_HC_F404TAG_Hole
MEVQLVESGGGLVQPGGSLRLSCAASGFNITSYGIHWVRQAPGKGLEWVGWIAPNSGNTYYADSVKGRFTISADTSKNTAYLQMNSLRAEDTAVYYCARDIVSTYSYYYLMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFLVSKLTVDKSRWQQGNVFSCSVMHEALHNHYTQKSLSLSPGK

SEQ ID NO: 1048 - protein, 444 aa, synthetic construct
  REGION 1..444: note = Synthetic: 2188-D04 IgG HC Y180/F241/F404TAG P011531_0
MEVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSSASTKGPSVFPLAPSSKSTSGGTAALGCLVKDYFPEPVTVSWNSGALTSGVHTFPAVLQSSGLSLSSVVTVPSSSLGTQTYICNVNHKPSNTKVDKKVEPKSCDKTHTCPPCPAPELLGGPSVLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLYSKLTVDKSRWQQGNVFSCSVMHEALHNHYTQKSLSLSPGK

SEQ ID NO: 1049 - protein, 213 aa, synthetic construct
  REGION 1..213: note = Synthetic: trastuzumab LC SerOpt K42TAG/E161TAG/TCT162AGC
MDIQMTQSPSSLSASVGDRVTITCRASQDVNTAVAWYQQKPGAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQSVTEQDSKDSTYSLSSTLTLSKADYEKHKVYACEVTHQGLSSPVTKSFNRGEC

SEQ ID NO: 1050 - protein, 214 aa, synthetic construct
  REGION 1..214: note = Synthetic: trastuzumab LC SerOpt K42TAG
MDIQMTQSPSSLSASVGDRVTITCRASQDVNTAVAWYQQKPGAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1051 - protein, 215 aa, synthetic construct
  REGION 1..215: note = Synthetic: Trastuzumab LC SerOpt
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1052 - protein, 447 aa, synthetic construct
  REGION 1..447: note = Synthetic: 2188-D04 HC
MEVQLVESGGGLVQPGGSLRLSCAASGFNISDYDIHWVRQAPGKGLEWVGGIDPADGSTDYADSVKGRFTISADTSKNTAYLQMNSLRAEDTAVYYCARDYGVYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1053 - protein, 215 aa, synthetic construct; identical to SEQ ID NO: 1051
  REGION 1..215: note = Synthetic: 2188-D04 LC